(19) United States
(12) Patent Application Publication
(10) Pub. No.: US 2025/0374748 A1
(43) Pub. Date: Dec. 4, 2025

(54) HETEROCYCLIC COMPOUND, ORGANIC LIGHT EMITTING DEVICE COMPRISING THE SAME AND COMPOSITION FOR ORGANIC MATERIAL LAYER

(71) Applicant: LT MATERIALS CO.,LTD., Yongin-si (KR)

(72) Inventors: Jeong A KIM, Yongin-si (KR); Jun Tae MO, Yongin-si (KR); Dong Jun KIM, Yongin-si (KR); Dae Hyuk CHOI, Yongin-si (KR)

(21) Appl. No.: 19/194,049

(22) Filed: Apr. 30, 2025

(30) Foreign Application Priority Data
May 29, 2024 (KR) 10-2024-0069888

(51) Int. Cl. H10K 50/12; H10K 85/30; H10K 85/60; H10K 101/00; H10K 101/25; H10K 102/00

(52) U.S. Cl. CPC H10K 50/12; H10K 85/324; H10K 85/342; H10K 85/626; H10K 85/633; H10K 85/636; H10K 85/654; H10K 85/6572; H10K 85/6574; H10K 85/6576; H10K 2101/25; H10K 2101/90; H10K 2102/321

(57) ABSTRACT

Provided is a heterocyclic compound represented by Chemical Formula 1, an organic light emitting device including the same, and a composition for an organic material layer. When the heterocyclic compound represented by Chemical Formula 1 is used in an organic material layer, itis possible to lower a driving voltage of an organic light emitting device, and improve light emission efficiency and lifetime properties thereof.

### TECHNICAL FIELD

This application claims priority to Korean Patent Application No. 10-2024-0069888, filed on May 29, 2024, the disclosure of which is incorporated herein by reference in its entirety.

The present disclosure relates to a heterocyclic compound, an organic light emitting device including the same, and a composition for an organic material layer.

### DESCRIPTION OF THE RELATED ART

An organic light emitting device is one type of self-emissive display devices, and has advantages of having a wide viewing angle and a high response speed as well as having an excellent contrast.

The organic light emitting device has a structure of disposing an organic thin film between two electrodes. When a voltage is applied to the organic light emitting device having such a structure, electrons and holes injected from the two electrodes bind and pair in the organic thin film, and then light is emitted as these annihilate. The organic thin film may be formed in a single layer or a multilayer as necessary.

A material of the organic thin film may have a light emitting function as necessary. For example, as a material of the organic thin film, compounds each capable of forming a light emitting layer themselves alone may be used, or compounds each capable of serving as a host or a dopant of a host-dopant-based light emitting layer may also be used. In addition thereto, compounds capable of performing roles of hole injection, hole transport, electron blocking, hole blocking, electron transport, electron injection and the like may also be used as a material of the organic thin film.

Development of an organic thin film material has been continuously required for enhancing performance, lifetime or efficiency of an organic light emitting device.

### PRIOR ART DOCUMENTS

### Patent Documents

(Patent Document 1) U.S. Pat. No. 4,356,429

### SUMMARY

The present disclosure is directed to providing a heterocyclic compound, an organic light emitting device including the same, and a composition for an organic material layer.

One embodiment of the present disclosure provides a heterocyclic compound represented by the following Chemical Formula 1.

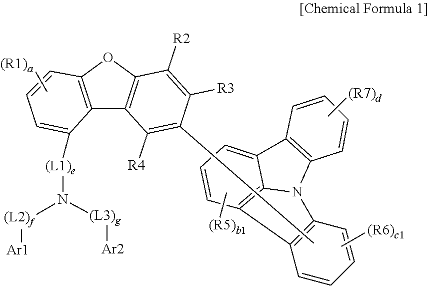

In Chemical Formula 1,
R1 to R7 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and —NR101R102, or two or more groups adjacent to each other bond to each other to form a substituted or unsubstituted C6 to C60 aromatic hydrocarbon ring; or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group,
a is an integer of 0 to 3, and when a is 2 or greater, R1s are the same as or different from each other,
b1 is an integer of 0 to 3, and when b1 is 2 or greater, R5s are the same as or different from each other,
c1 is an integer of 0 to 4, and when c1 is 2 or greater, R6s are the same as or different from each other,
d is an integer of 0 to 4, and when d is 2 or greater, R7s are the same as or different from each other,
L1 to L3 are the same as or different from each other, and each independently a direct bond; a substituted or unsubstituted C6 to C60 arylene group; or a substituted or unsubstituted C2 to C60 heteroarylene group,
e is an integer of 0 to 5, and when e is 2 or greater, L1s are the same as or different from each other,
f is an integer of 0 to 5, and when f is 2 or greater, L2s are the same as or different from each other,
g is an integer of 0 to 5, and when g is 2 or greater, L3s are the same as or different from each other, and
Ar1 and Ar2 are the same as or different from each other, and each independently a substituted or unsubstituted C6 to C60 aryl group; or a substituted or unsubstituted C2 to C60 heteroaryl group.

In addition, one embodiment of the present disclosure provides an organic light emitting device comprising:
a first electrode;
a second electrode provided opposite to the first electrode; and
one or more organic material layers provided between the first electrode and the second electrode,
wherein one or more layers of the one or more organic material layers comprise the heterocyclic compound represented by Chemical Formula 1.

In addition, one embodiment of the present disclosure provides an organic light emitting device, wherein the organic material layer further includes a heterocyclic compound represented by the following Chemical Formula 2.

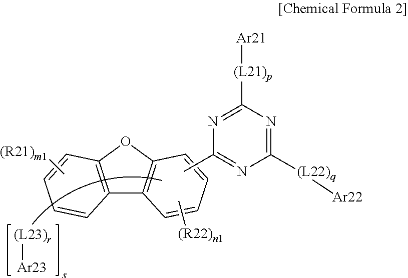

In Chemical Formula 2,
R21 and R22 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; —P(═O)R201R202; —SiR201R202R203; and —NR201R202, or two or more groups adjacent to each other bond to each other to form a substituted or unsubstituted C6 to C60 aromatic hydrocarbon ring; or a substituted or unsubstituted C2 to C60 heteroring, and R201, R202 and R203 are the same as or different from each other and each independently a substituted or unsubstituted C1 to C60 alkyl group; a substituted or unsubstituted C6 to C60 aryl group; or a substituted or unsubstituted C2 to C60 heteroaryl group,
m1 is an integer of 0 to 4, and when m1 is 2 or greater, R21s are the same as or different from each other,
n1 is an integer of 0 to 3, and when n1 is 2 or greater, R22s are the same as or different from each other,
L21 to L23 are the same as or different from each other, and each independently a direct bond; a substituted or unsubstituted C6 to C60 arylene group; or a substituted or unsubstituted C2 to C60 heteroarylene group,
p is an integer of 0 to 5, and when p is 2 or greater, L21s are the same as or different from each other,
q is an integer of 0 to 5, and when q is 2 or greater, L22s are the same as or different from each other,
r is an integer of 0 to 5, and when r is 2 or greater, L23s are the same as or different from each other,
Ar21 to Ar23 are the same as or different from each other, and each independently a substituted or unsubstituted C6 to C60 aryl group; or a substituted or unsubstituted C2 to C60 heteroaryl group, and
s is 0 or 1.

In addition, one embodiment of the present disclosure provides a composition for an organic material layer, the composition comprising: the heterocyclic compound represented by Chemical Formula 1; and the heterocyclic compound represented by Chemical Formula 2.

The compound described in the present specification can be used as an organic material layer material of an organic light emitting device. The compound is capable of performing roles of a hole injection layer material, a hole transport layer material, a light emitting layer material, an electron transport layer material, an electron injection layer material and the like in an organic light emitting device. Particularly, the compound can be used as a light emitting layer material of an organic light emitting device. The compound can be used alone as a light emitting material, or can be used as a host material or a dopant material of a light emitting layer.

When the heterocyclic compound represented by Chemical Formula 1 is used in an organic material layer, it is possible to lower a driving voltage of an organic light emitting device, and improve light emission efficiency and lifetime properties thereof.

### BRIEF DESCRIPTION OF TIE DRAWINGS

FIGS. 1 to 3 are diagrams each schematically illustrating a lamination structure of an organic light emitting device according to one embodiment of the present disclosure.

### DESCRIPTION OF SPECIFIC EMBODIMENTS

Hereinafter, the present disclosure will be described in more detail.

In the present specification, a term “substitution” means that a hydrogen atom bonding to a carbon atom of a compound is changed to another substituent, and the position of substitution is not limited as long as it is a position at which the hydrogen atom is substituted, that is, a position at which a substituent is capable of substituting, and when two or more substituents substitute, the two or more substituents may be the same as or different from each other.

In the present specification, “substituted or unsubstituted” means being substituted with one or more substituents selected from the group consisting of deuterium; halogen; a cyano group; a C1 to C60 linear or branched alkyl group; a C2 to C60 linear or branched alkenyl group; a C2 to C60 linear or branched alkynyl group; a C1 to C60 linear, branched or cyclic alkoxy group; a C3 to C60 monocyclic or polycyclic cycloalkyl group; a C2 to C60 monocyclic or polycyclic heterocycloalkyl group; a C6 to C60 monocyclic or polycyclic aryl group; a C2 to C60 monocyclic or polycyclic heteroaryl group; —SiRR′R″; —P(═O)RR′; a C1 to C20 alkylamine group; a C6 to C60 monocyclic or polycyclic arylamine group; and a C2 to C60 monocyclic or polycyclic heteroarylamine group or being unsubstituted, or being substituted with a substituent in which two or more substituents selected from among the substituents exemplified above are linked or being unsubstituted, and R, R′ and R″ may be a substituted or unsubstituted C1 to C60 alkyl group; a substituted or unsubstituted C6 to C60 aryl group; or a substituted or unsubstituted C2 to C60 heteroaryl group.

In the present specification, the halogen may be fluorine; chlorine; bromine; or iodine.

In the present specification, the alkyl group includes a linear or branched form having 1 to 60 carbon atoms, and may be further substituted with other substituents. The number of carbon atoms of the alkyl group may be from 1 to 60, specifically from 1 to 40 and more specifically from 1 to 20. Specific examples of the alkyl group may include a methyl group; an ethyl group; an n-propyl group; an isopropyl group; an n-butyl group; an isobutyl group; a tert-butyl group; a sec-butyl group; a 1-methyl-butyl group; a 1-ethyl-butyl group; an n-pentyl group; an isopentyl group; a neopentyl group; a tert-pentyl group; an n-hexyl group; a 1-methylpentyl group; a 2-methylpentyl group; a 4-methyl-2-pentyl group; a 3,3-dimethylbutyl group; a 2-ethylbutyl group; an n-heptyl group; a 1-methylhexyl group; a cyclopentylmethyl group; a cyclohexylmethyl group; an n-octyl group; a tert-octyl group; a 1-methylheptyl group; a 2-ethylhexyl group; a 2-propylpentyl group; an n-nonyl group; a 2,2-dimethylheptyl group; a 1-ethyl-propyl group; a 1,1-dimethyl-propyl group; an isohexyl group; a 4-methylhexyl group; a 5-methylhexyl group and the like, but are not limited thereto.

In the present specification, the alkenyl group includes a linear or branched form having 2 to 60 carbon atoms, and may be further substituted with other substituents. The number of carbon atoms of the alkenyl group may be from 2 to 60, specifically from 2 to 40 and more specifically from 2 to 20. Specific examples of the alkenyl group may include a vinyl group; a 1-propenyl group; an isopropenyl group; a 1-butenyl group; a 2-butenyl group; a 3-butenyl group; a 1-pentenyl group; a 2-pentenyl group; a 3-pentenyl group; a 3-methyl-1-butenyl group; a 1,3-butadienyl group; an allyl group; a 1-phenylvinyl-1-yl group; a 2-phenylvinyl-1-yl group; a 2,2-diphenylvinyl-1-yl group; a 2-phenyl-2-(naphthyl-1-yl)vinyl-1-yl group; a 2,2-bis(diphenyl-1-yl)vinyl-1-yl group; a stilbenyl group; a styrenyl group and the like, but are not limited thereto.

In the present specification, the alkynyl group includes a linear or branched form having 2 to 60 carbon atoms, and may be further substituted with other substituents. The number of carbon atoms of the alkynyl group may be from 2 to 60, specifically from 2 to 40 and more specifically from 2 to 20.

In the present specification, the alkoxy group may be linear, branched or cyclic. The number of carbon atoms of the alkoxy group is not particularly limited, but is preferably from 1 to 20. Specific examples of the alkoxy group may include a methoxy group; an ethoxy group; an n-propoxy group; an isopropoxy group; an n-butoxy group; an isobutoxy group; a tert-butoxy group; a sec-butoxy group; an n-pentyloxy group; a neopentyloxy group; an isopentyloxy group; an n-hexyloxy group; a 3,3-dimethylbutyloxy group; a 2-ethylbutyloxy group; an n-octyloxy group; an n-nonyloxy group; an n-decyloxy group; a benzyloxy group; a p-methylbenzyloxy group and the like, but are not limited thereto.

In the present specification, the cycloalkyl group includes a monocyclic or polycyclic group having 3 to 60 carbon atoms, and may be further substituted with other substituents. Herein, the polycyclic group means a group in which the cycloalkyl group is directly linked to or fused with another cyclic group. Herein, the another cyclic group may be a cycloalkyl group, but may also be different types of cyclic groups such as a heterocycloalkyl group; an aryl group; and a heteroaryl group. The number of carbon atoms of the cycloalkyl group may be from 3 to 60, specifically from 3 to 40 and more specifically from 5 to 20. Specific examples of the cycloalkyl group may include a cyclopropyl group; a cyclobutyl group; a cyclopentyl group; a 3-methylcyclopentyl group; a 2,3-dimethylcyclopentyl group; a cyclohexyl group; a 3-methylcyclohexyl group; a 4-methylcyclohexyl group; a 2,3-dimethylcyclohexyl group; a 3,4,5-trimethylcyclohexyl group; a 4-tert-butylcyclohexyl group; a cycloheptyl group; a cyclooctyl group and the like, but are not limited thereto.

In the present specification, the heterocycloalkyl group includes O, S, Se, N or Si as a heteroatom, includes a monocyclic or polycyclic group having 2 to 60 carbon atoms, and may be further substituted with other substituents. Herein, the polycyclic group means a group in which the heterocycloalkyl group is directly linked to or fused with another cyclic group. Herein, the another cyclic group may be a heterocycloalkyl group, but may also be different types of cyclic groups such as a cycloalkyl group; an aryl group; and a heteroaryl group. The number of carbon atoms of the heterocycloalkyl group may be from 2 to 60, specifically from 2 to 40 and more specifically from 3 to 20.

In the present specification, the aryl group includes a monocyclic or polycyclic group having 6 to 60 carbon atoms, and may be further substituted with other substituents. Herein, the polycyclic group means a group in which the aryl group is directly linked to or fused with another cyclic group. Herein, the another cyclic group may be an aryl group, but may also be different types of cyclic groups such as a cycloalkyl group; a heterocycloalkyl group; and a heteroaryl group. The aryl group may include a spiro group. The number of carbon atoms of the aryl group may be from 6 to 60, specifically from 6 to 40 and more specifically from 6 to 20. Specific examples of the aryl group may include a phenyl group; a biphenyl group; a triphenyl group; a naphthyl group; an anthryl group; a chrysenyl group; a phenanthrenyl group; a perylenyl group; a fluoranthenyl group; a triphenylenyl group; a phenalenyl group; a pyrenyl group; a tetracenyl group; a pentacenyl group; a fluorenyl group; an indenyl group; an acenaphthylenyl group; a benzofluorenyl group; a spirobifluorenyl group; a 2,3-dihydro-1H-indenyl group; a fused ring group thereof, and the like, but are not limited thereto.

In the present specification, the phosphine oxide group is represented by —P(═O)R101R102, and R101 and R102 are the same as or different from each other and may be each independently a substituent formed with at least one of hydrogen; deuterium; a halogen group; a substituted or unsubstituted C1 to C60 alkyl group; a substituted or unsubstituted C2 to C60 alkenyl group; a substituted or unsubstituted C1 to C60 alkoxy group; a substituted or unsubstituted C3 to C60 monocyclic or polycyclic cycloalkyl group; a substituted or unsubstituted C6 to C60 monocyclic or polycyclic aryl group; and a substituted or unsubstituted C2 to C60 monocyclic or polycyclic heteroaryl group. Specifically, the phosphine oxide group may be substituted with a substituted or unsubstituted C6 to C60 monocyclic or polycyclic aryl group, and as the aryl group, the examples described above may be applied. Examples of the phosphine oxide group may include a diphenylphosphine oxide group; a dinaphthylphosphine oxide group and the like, but are not limited thereto.

In the present specification, the silyl group is a substituent including Si and having the Si atom directly linked as a radical, and is represented by —SiR101R102R103. R101 to R103 are the same as or different from each other, and may be each independently a substituent formed with at least one of hydrogen; deuterium; a halogen group; a substituted or unsubstituted C1 to C60 alkyl group; a substituted or unsubstituted C2 to C60 alkenyl group; a substituted or unsubstituted C1 to C60 alkoxy group; a substituted or unsubstituted C3 to C60 monocyclic or polycyclic cycloalkyl group; a substituted or unsubstituted C6 to C60 monocyclic or polycyclic aryl group; and a substituted or unsubstituted C2 to C60 monocyclic or polycyclic heteroaryl group. Specific examples of the silyl group may include a trimethylsilyl group; a triethylsilyl group; a t-butyldimethylsilyl group; a vinyldimethylsilyl group; a propyldimethylsilyl group; a triphenylsilyl group; a diphenylsilyl group; a phenylsilyl group and the like, but are not limited thereto.

In the present specification, the fluorenyl group may be substituted, and adjacent substituents may bond to each other to form a ring.

When the fluorenyl group is substituted,

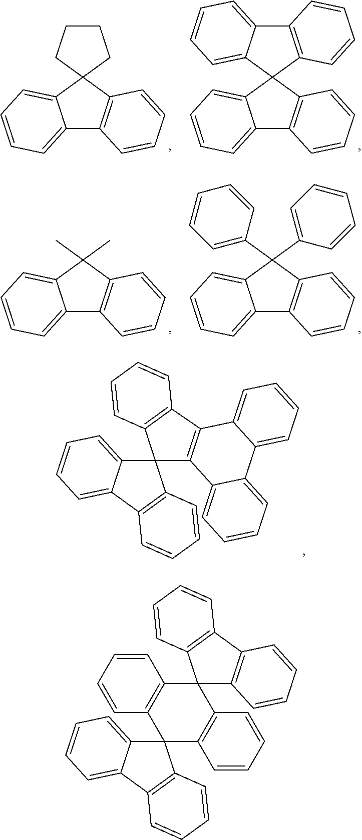

and the like may be included, however, the structure is not limited thereto.

In the present specification, the spiro group is a group including a spiro structure, and may have 15 to 60 carbon atoms. For example, the spiro group may include a structure in which a 2,3-dihydro-1H-indene group or a cyclohexane group spiro bonds to a fluorenyl group. Specifically, the spiro group may include any one of groups of the following structural formulae.

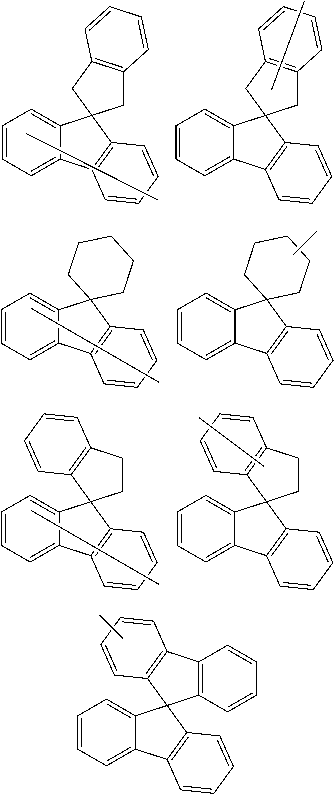

In the present specification, the heteroaryl group includes S; O; Se; N; or Si as a heteroatom, includes a monocyclic or polycyclic group having 2 to 60 carbon atoms, and may be further substituted with other substituents. Herein, the polycyclic group means a group in which the heteroaryl group is directly linked to or fused with another cyclic group. Herein, the another cyclic group may be a heteroaryl group, but may also be different types of cyclic groups such as a cycloalkyl group; a heterocycloalkyl group; and an aryl group. The number of carbon atoms of the heteroaryl group may be from 2 to 60, specifically from 2 to 40 and more specifically from 3 to 25. Specific examples of the heteroaryl group may include a pyridyl group; a pyrrolyl group; a pyrimidyl group; a pyridazinyl group; a furanyl group; a thiophenyl group; an imidazolyl group; a pyrazolyl group; an oxazolyl group; an isoxazolyl group; a thiazolyl group; an isothiazolyl group; a triazolyl group; a furazanyl group; an oxadiazolyl group; a thiadiazolyl group; a dithiazolyl group; a tetrazolyl group; a pyranyl group; a thiopyranyl group; a diazinyl group; an oxazinyl group; a thiazinyl group; a dioxynyl group; a triazinyl group; a tetrazinyl group; a quinolyl group; an isoquinolyl group; a quinazolinyl group; an isoquinazolinyl group; a quinozolinyl group; a naphthyridyl group; an acridinyl group; a phenanthridinyl group; an imidazopyridinyl group; a diazanaphthalenyl group; a triazaindenyl group; a 2-indolyl group; an indolizinyl group; a benzothiazolyl group; a benzoxazolyl group; a benzimidazolyl group; a benzothiophenyl group; a benzofuranyl group; a dibenzothiophenyl group; a dibenzofuranyl group; a carbazolyl group; a benzocarbazolyl group; a dibenzocarbazolyl group; a phenazinyl group; a dibenzosilole group; a spirobi(dibenzosilole) group; a dihydrophenazinyl group; a phenoxazinyl group; a phenanthridyl group; a thienyl group; an indolo[2,3-a]carbazolyl group; an indolo[2,3-b]carbazolyl group; an indolinyl group; a 10,11-dihydro-dibenzo[b,f]azepinyl group; a 9,10-dihydroacridinyl group; a phenanthrazinyl group; a phenothiazinyl group; a phthalazinyl group; a naphthylidinyl group; a phenanthrolinyl group; a benzo[c][1,2,5]thiadiazolyl group; a 5,10-dihydrodibenzo[b,e][1,4]azasilinyl group; a pyrazolo[1,5-c]quinazolinyl group; a pyrido[1,2-b]indazolyl group; a pyrido[1,2-a]imidazo[1,2-e]indolinyl group; a 5,11-dihydroindeno[1,2-b]carbazolyl group and the like, but are not limited thereto.

In the present specification, the amine group may be selected from the group consisting of a monoalkylamine group; a monoarylamine group; a monoheteroarylamine group; —NH₂; a dialkylamine group; a diarylamine group; a diheteroarylamine group; an alkylarylamine group; an alkylheteroarylamine group; and an arylheteroarylamine group, and the number of carbon atoms is not particularly limited, but preferably from 1 to 30. Specific examples of the amine group may include a methylamine group; a dimethylamine group; an ethylamine group; a diethylamine group; a phenylamine group; a naphthylamine group; a biphenylamine group; a dibiphenylamine group; an anthracenylamine group; a 9-methyl-anthracenylamine group; a diphenylamine group; a phenylnaphthylamine group; a ditolylamine group; a phenyltolylamine group; a triphenylamine group; a biphenylnaphthylamine group; a phenylbiphenylamine group; a biphenylfluorenylamine group; a phenyltriphenylenylamine group; a biphenyltriphenylenylamine group and the like, but are not limited thereto.

In the present specification, the arylene group means the aryl group having two bonding sites, that is, a divalent group. The descriptions on the aryl group provided above may be applied thereto except for those that are each a divalent group. In addition, the heteroarylene group means the heteroaryl group having two bonding sites, that is, a divalent group. The descriptions on the heteroaryl group provided above may be applied thereto except for those that are each a divalent group.

In the present specification, an “adjacent” group may mean a substituent substituting an atom directly linked to an atom substituted by the corresponding substituent, a substituent sterically most closely positioned to the corresponding substituent, or another substituent substituting an atom substituted by the corresponding substituent. For example, two substituents substituting at ortho positions in a benzene ring, and two substituents substituting at the same carbon in an aliphatic ring may be interpreted as groups “adjacent” to each other.

In the present disclosure, a “case of a substituent being not indicated in a chemical formula or compound structure” means that a hydrogen atom bonds to a carbon atom. However, since deuterium (2H, D) is an isotope of hydrogen, some hydrogen atoms may be deuterium.

In one embodiment of the present disclosure, a “case of a substituent being not indicated in a chemical formula or compound structure” may mean that positions to which substituents may come are all hydrogen or deuterium. In other words, since deuterium is an isotope of hydrogen, some hydrogen atoms may be deuterium that is an isotope, and herein, a content of the deuterium may be from 0% to 100%.

In one embodiment of the present disclosure, in a “case of a substituent being not indicated in a chemical formula or compound structure”, hydrogen and deuterium may be used interchangeably in compounds when deuterium is not explicitly excluded such as “a deuterium content being 00”, “a hydrogen content being 1000%0” or “substituents being all hydrogen”.

In one embodiment of the present disclosure, deuterium is one of isotopes of hydrogen, is an element having deuteron formed with one proton and one neutron as a nucleus, and may be expressed as hydrogen-2, and the elemental symbol thereof may also be written as D or ²H.

In one embodiment of the present disclosure, an isotope means an atom with the same atomic number (Z) but with a different mass number (A), and may also be interpreted as an element with the same number of protons but with a different number of neutrons.

In one embodiment of the present disclosure, a content T % of a specific substituent may be defined as T2/T1×100=T % when the total number of substituents that a basic compound may have is defined as T1, and the number of specific substituents among these is defined as T2.

In other words, in one example, having a deuterium content of 20% in a phenyl group represented by

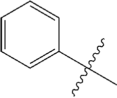 [SMILES: *c1ccccc1]

may mean that the total number of substituents that the phenyl group may have is 5 (T1 in the formula), and the number of deuterium atoms among these is 1 (T2 in the formula). In other words, having a deuterium content of 20% in a phenyl group may be represented by the following structural formulae.

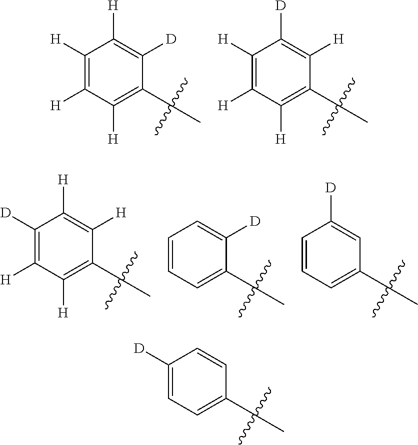 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

In addition, in one embodiment of the present disclosure, “a phenyl group having a deuterium content of 00%” may mean a phenyl group that does not include a deuterium atom, that is, a phenyl group that has 5 hydrogen atoms.

In the present disclosure, the C6 to C60 aromatic hydrocarbon ring means a compound including an aromatic ring formed with C6 to C60 carbons and hydrogens. Examples thereof may include phenyl, biphenyl, terphenyl, triphenylene, naphthalene, anthracene, phenalene, phenanthrene, fluorene, pyrene, chrysene, perylene, azulene and the like, but are not limited thereto, and include all aromatic hydrocarbon ring compounds known in the art satisfying the above-mentioned number of carbon atoms.

One embodiment of the present disclosure provides a heterocyclic compound represented by the following Chemical Formula 1.

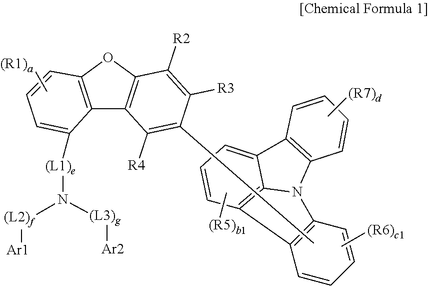

In Chemical Formula 1,
R1 to R7 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and —NR101R102, or two or more groups adjacent to each other bond to each other to form a substituted or unsubstituted C6 to C60 aromatic hydrocarbon ring; or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group, a is an integer of 0 to 3, and when a is 2 or greater, R1s are the same as or different from each other,
b1 is an integer of 0 to 3, and when b1 is 2 or greater, R5s are the same as or different from each other,
c1 is an integer of 0 to 4, and when c1 is 2 or greater, R6s are the same as or different from each other,
d is an integer of 0 to 4, and when d is 2 or greater, R7s are the same as or different from each other,
L1 to L3 are the same as or different from each other, and each independently a direct bond; a substituted or unsubstituted C6 to C60 arylene group; or a substituted or unsubstituted C2 to C60 heteroarylene group,
e is an integer of 0 to 5, and when e is 2 or greater, L1s are the same as or different from each other,
f is an integer of 0 to 5, and when f is 2 or greater, L2s are the same as or different from each other, g is an integer of 0 to 5, and when g is 2 or greater, L3s are the same as or different from each other, and
Ar1 and Ar2 are the same as or different from each other, and each independently a substituted or unsubstituted C6 to C60 aryl group; or a substituted or unsubstituted C2 to C60 heteroaryl group.

In one embodiment of the present disclosure, R1 to R7 are the same as or different from each other, and each independently hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; —P(═O)R101R102; —SiR101R102R103; or —NR101R102, or two or more groups adjacent to each other bond to each other to form a substituted or unsubstituted C6 to C30 aromatic hydrocarbon ring; or a substituted or unsubstituted C2 to C30 heteroring, and R101, R102 and R103 are the same as or different from each other and may be each independently a substituted or unsubstituted C1 to C30 alkyl group; a substituted or unsubstituted C6 to C30 aryl group; or a substituted or unsubstituted C2 to C30 heteroaryl group.

In another embodiment of the present disclosure, R1 to R7 are the same as or different from each other, and each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; a substituted or unsubstituted C2 to C20 heteroaryl group; —P(═O)R101R102; —SiR101R102R103; or —NR101R102, or two or more groups adjacent to each other bond to each other to form a substituted or unsubstituted C6 to C20 aromatic hydrocarbon ring; or a substituted or unsubstituted C2 to C20 heteroring, and R101, R102 and R103 are the same as or different from each other and may be each independently a substituted or unsubstituted C1 to C20 alkyl group; a substituted or unsubstituted C6 to C20 aryl group; or a substituted or unsubstituted C2 to C20 heteroaryl group.

In another embodiment of the present disclosure, R1 to R7 are the same as or different from each other, and each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; or a substituted or unsubstituted C2 to C20 heteroaryl group, or two or more groups adjacent to each other bond to each other to form a substituted or unsubstituted C6 to C20 aromatic hydrocarbon ring; or a substituted or unsubstituted C2 to C20 heteroring.

In another embodiment of the present disclosure, R1 to R7 are the same as or different from each other, and each independently hydrogen; deuterium; halogen; a cyano group; a substituted or unsubstituted C1 to C20 alkyl group; a substituted or unsubstituted C6 to C20 aryl group; or a substituted or unsubstituted C2 to C20 heteroaryl group, or two or more groups adjacent to each other may bond to each other to form a substituted or unsubstituted C6 to C20 aromatic hydrocarbon ring; or a substituted or unsubstituted C2 to C20 heteroring.

In another embodiment of the present disclosure, R1 to R7 are the same as or different from each other, and each independently hydrogen; or deuterium, or two or more groups adjacent to each other may bond to each other to form a substituted or unsubstituted C6 to C20 aromatic hydrocarbon ring; or a substituted or unsubstituted C2 to C20 heteroring.

In another embodiment of the present disclosure, R1 to R4 are the same as or different from each other, and each independently hydrogen; or deuterium, or two or more groups adjacent to each other may bond to each other to form a substituted or unsubstituted C6 to C20 aromatic hydrocarbon ring; or a substituted or unsubstituted C2 to C20 heteroring.

In another embodiment of the present disclosure, R1 and R4 are the same as or different from each other, and may be each independently hydrogen; or deuterium.

In another embodiment of the present disclosure, R2 and R3 are the same as or different from each other, and each independently hydrogen; or deuterium, or two or more groups adjacent to each other may bond to each other to form a substituted or unsubstituted C6 to C20 aromatic hydrocarbon ring; or a substituted or unsubstituted C2 to C20 heteroring.

In another embodiment of the present disclosure, R2 and R3 are the same as or different from each other, and each independently hydrogen; or deuterium, or two or more groups adjacent to each other may bond to each other to form a substituted or unsubstituted benzene ring.

In another embodiment of the present disclosure, R2 and R3 are hydrogen, or two or more groups adjacent to each other may bond to each other to form a substituted or unsubstituted C6 to C20 aromatic hydrocarbon ring; or a substituted or unsubstituted C2 to C20 heteroring.

In another embodiment of the present disclosure, R2 and R3 are hydrogen, or two or more groups adjacent to each other may bond to each other to form a substituted or unsubstituted benzene ring.

In another embodiment of the present disclosure, R5 to R7 are the same as or different from each other, and may be each independently hydrogen; or deuterium.

In one embodiment of the present disclosure, L1 to L3 are the same as or different from each other, and may be each independently a direct bond; a substituted or unsubstituted C6 to C30 arylene group; or a substituted or unsubstituted C2 to C30 heteroarylene group.

In another embodiment of the present disclosure, L1 to L3 are the same as or different from each other, and may be each independently a direct bond; a substituted or unsubstituted C6 to C20 arylene group; or a substituted or unsubstituted C2 to C20 heteroarylene group.

In another embodiment of the present disclosure, L1 may be a direct bond; or a substituted or unsubstituted C6 to C20 arylene group.

In another embodiment of the present disclosure, L1 may be a direct bond; a substituted or unsubstituted phenylene group; or a substituted or unsubstituted naphthylene group.

In another embodiment of the present disclosure, L2 and L3 are the same as or different from each other, and may be each independently a direct bond; a substituted or unsubstituted C6 to C20 arylene group; or a substituted or unsubstituted C2 to C20 heteroarylene group.

In another embodiment of the present disclosure, L2 and L3 are the same as or different from each other, and may be each independently a direct bond; a substituted or unsubstituted phenylene group; a substituted or unsubstituted biphenylene group; a substituted or unsubstituted naphthylene group; or a substituted or unsubstituted carbazolene group.

In one embodiment of the present disclosure, Ar1 and Ar2 are the same as or different from each other, and may be each independently a substituted or unsubstituted C6 to C30 aryl group; or a substituted or unsubstituted C2 to C30 heteroaryl group.

In another embodiment of the present disclosure, Ar1 and Ar2 are the same as or different from each other, and may be each independently a substituted or unsubstituted C6 to C20 aryl group; or a substituted or unsubstituted C2 to C20 heteroaryl group.

In another embodiment of the present disclosure, Ar1 and Ar2 are the same as or different from each other, and may be each independently a substituted or unsubstituted phenyl group; a substituted or unsubstituted biphenyl group; a substituted or unsubstituted terphenyl group; a substituted or unsubstituted naphthyl group; a substituted or unsubstituted dibenzofuranyl group; a substituted or unsubstituted dibenzothiophenyl group; or a substituted or unsubstituted carbazolyl group.

In one embodiment of the present disclosure, the heterocyclic compound represented by Chemical Formula 1 may be represented by anyone of the following Chemical Formulae 1-1 and 1-2.

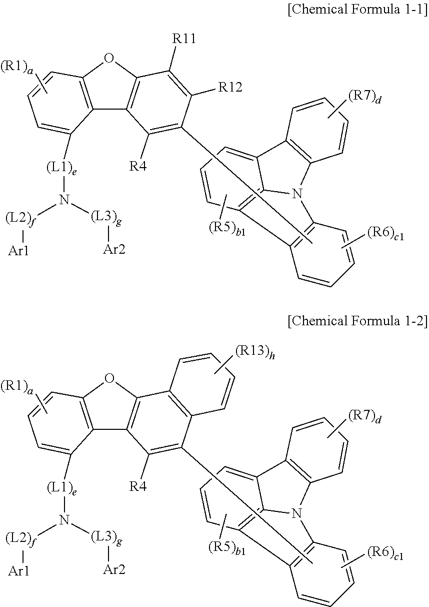

In Chemical Formulae 1-1 and 1-2,
R11 to R13 are the same as or different from each other and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and —NR101R102, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group,
h is an integer of 0 to 4, and when h is 2 or greater, R13s are the same as or different from each other, and
R1, R4 to R7, L1 to L3, Ar1, Ar2, a, b1, c1 and d to g have the same definitions as in Chemical Formula 1.

In one embodiment of the present disclosure, R11 to R13 are the same as or different from each other and each independently hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; —P(═O)R101R102; —SiR101R102R103; or —NR101R102, and R101, R102 and R103 are the same as or different from each other and may be each independently a substituted or unsubstituted C1 to C30 alkyl group; a substituted or unsubstituted C6 to C30 aryl group; or a substituted or unsubstituted C2 to C30 heteroaryl group.

In another embodiment of the present disclosure, R11 to R13 are the same as or different from each other and each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; a substituted or unsubstituted C2 to C20 heteroaryl group; —P(═O)R101R102; —SiR101R102R103; or —NR101R102, and R101, R102 and R103 are the same as or different from each other and may be each independently a substituted or unsubstituted C1 to C20 alkyl group; a substituted or unsubstituted C6 to C20 aryl group; or a substituted or unsubstituted C2 to C20 heteroaryl group.

In another embodiment of the present disclosure, R11 to R13 are the same as or different from each other, and may be each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; or a substituted or unsubstituted C2 to C20 heteroaryl group.

In another embodiment of the present disclosure, R11 to R13 are the same as or different from each other, and may be each independently hydrogen; deuterium; halogen; a cyano group; a substituted or unsubstituted C1 to C20 alkyl group; a substituted or unsubstituted C6 to C20 aryl group; or a substituted or unsubstituted C2 to C20 heteroaryl group.

In another embodiment of the present disclosure, R11 to R13 are the same as or different from each other, and may be each independently hydrogen; deuterium; a substituted or unsubstituted C6 to C20 aryl group; or a substituted or unsubstituted C2 to C20 heteroaryl group.

In another embodiment of the present disclosure, R11 to R13 are the same as or different from each other, and may be each independently hydrogen; or deuterium.

In one embodiment of the present disclosure, the heterocyclic compound represented by Chemical Formula 1-1 may be represented by the following Chemical Formula 1-1-1 or 1-1-2.

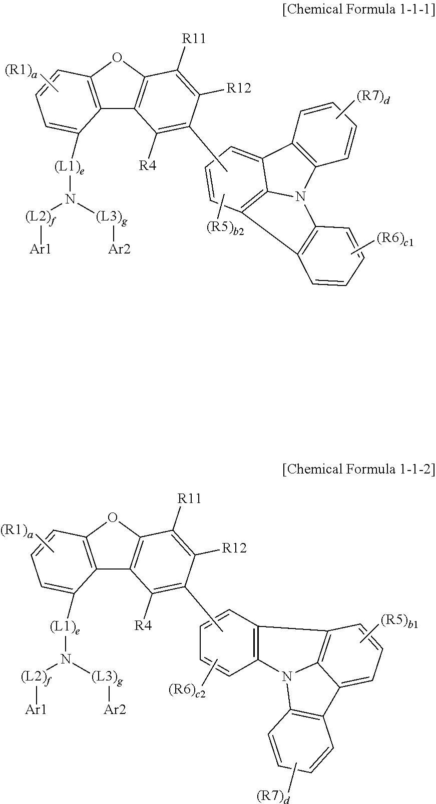

In Chemical Formulae 1-1-1 and 1-1-2,
b2 is an integer of 0 to 2, and when b2 is 2 or greater, R5s are the same as or different from each other,
c2 is an integer of 0 to 3, and when c2 is 2 or greater, R6s are the same as or different from each other,
R1, R4 to R7, L1 to L3, Ar1, Ar2, a, b1, c1 and d to g have the same definitions as in Chemical Formula 1, and
R11 and R12 have the same definitions as in Chemical Formula 1-1.

In one embodiment of the present disclosure, the heterocyclic compound represented by Chemical Formula 1-1-1 may be represented by any one of the following Chemical Formulae 1-1-1a to 1-1-1c.

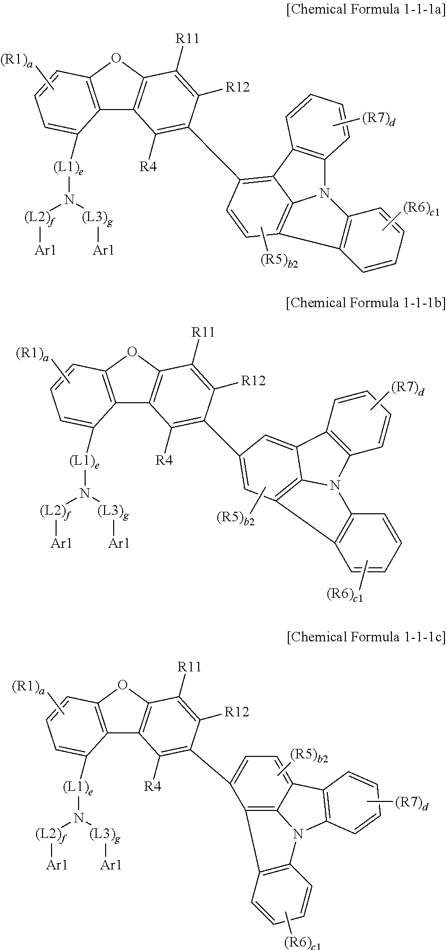

In Chemical Formulae 1-1-1a to 1-1-1c,
R1, R4 to R7, L1 to L3, Ar1, Ar2, a, c1 and d to g have the same definitions as in Chemical Formula 1,
R11 and R12 have the same definitions as in Chemical Formula 1-1, and
b2 has the same definition as in Chemical Formula 1-1-1.

In one embodiment of the present disclosure, the heterocyclic compound represented by Chemical Formula 1-1-2 may be represented by any one of the following Chemical Formulae 1-1-2a to 1-1-2d.

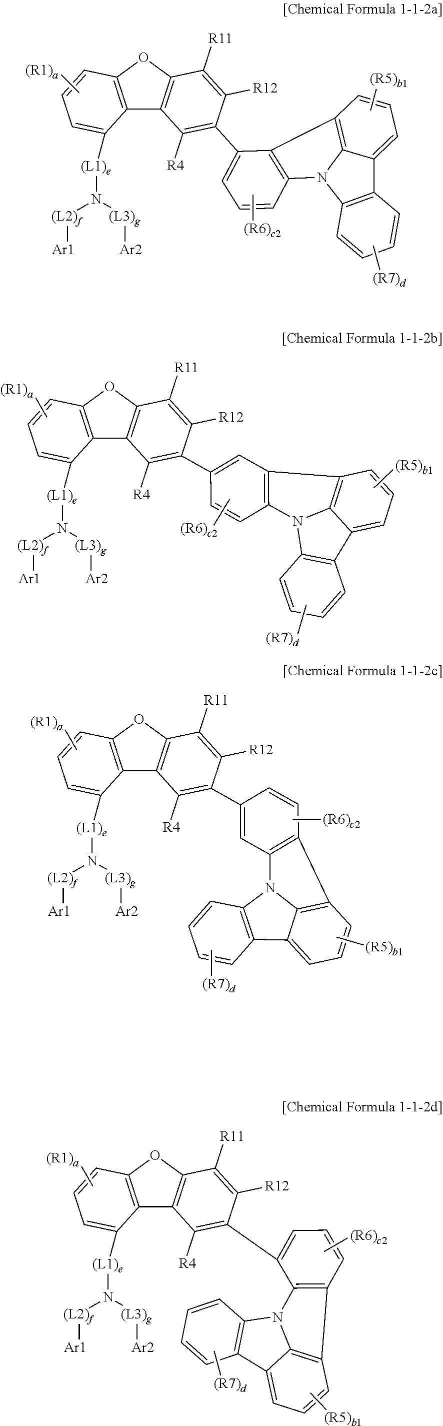

In Chemical Formulae 1-1-2a to 1-1-2d,
R1, R4 to R7, L1 to L3, Ar1, Ar2, a, b1 and d to g have the same definitions as in Chemical Formula 1,
R11 and R12 have the same definitions as in Chemical Formula 1-1, and
c2 has the same definition as in Chemical Formula 1-1-2.

In one embodiment of the present disclosure, the heterocyclic compound represented by Chemical Formula 1-2 may be represented by the following Chemical Formula 1-2-1 or 1-2-2.

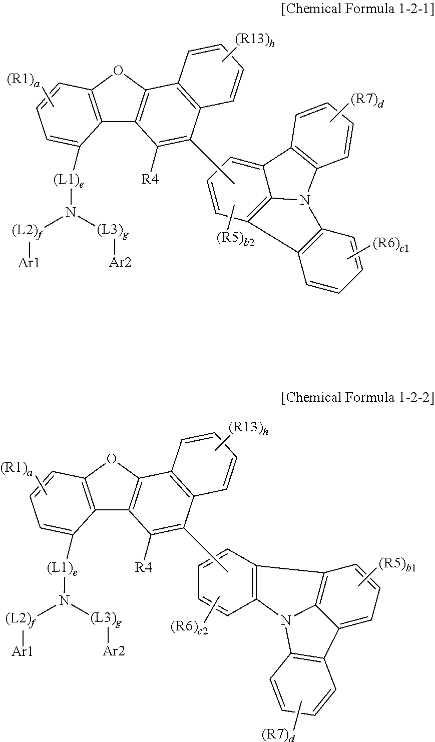

In Chemical Formulae 1-2-1 and 1-2-2,
b2 is an integer of 0 to 2, and when b2 is 2 or greater, R5s are the same as or different from each other,
c2 is an integer of 0 to 3, and when c2 is 2 or greater, R6s are the same as or different from each other,
R1, R4 to R7, L1 to L3, Ar1, Ar2, a, b1, c1 and d to f have the same definitions as in Chemical Formula 1, and R13 and h have the same definitions as in Chemical Formula 1-2.

In one embodiment of the present disclosure, the heterocyclic compound represented by Chemical Formula 1-2-1 may be represented by any one of the following Chemical Formulae 1-2-1a to 1-2-1c.

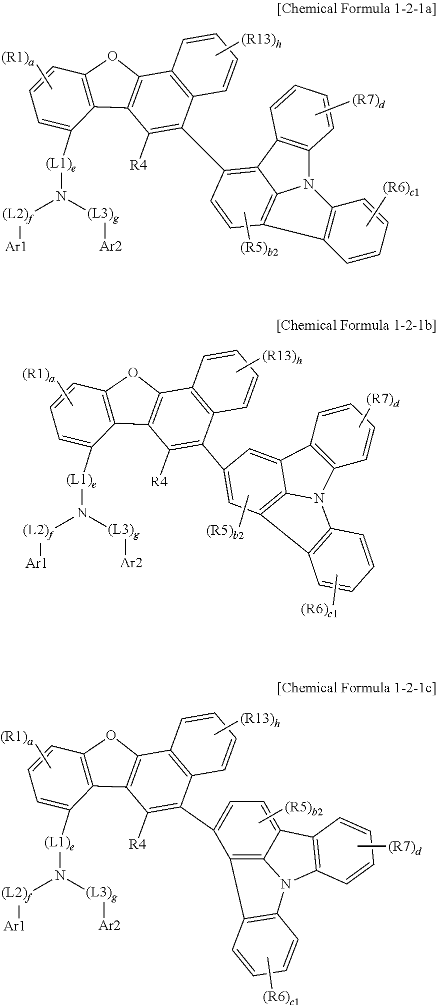

In Chemical Formulae 1-2-1a to 1-2-1c,
R1, R4 to R7, L1 to L3, Ar1, Ar2, a, c1 and d to g have the same definitions as in Chemical Formula 1,
R13 and h have the same definitions as in Chemical Formula 1-2, and
b2 has the same definition as in Chemical Formula 1-2-1.

In one embodiment of the present disclosure, the heterocyclic compound represented by Chemical Formula 1-2-2 may be represented by any one of the following Chemical Formulae 1-2-2a to 1-2-2d.

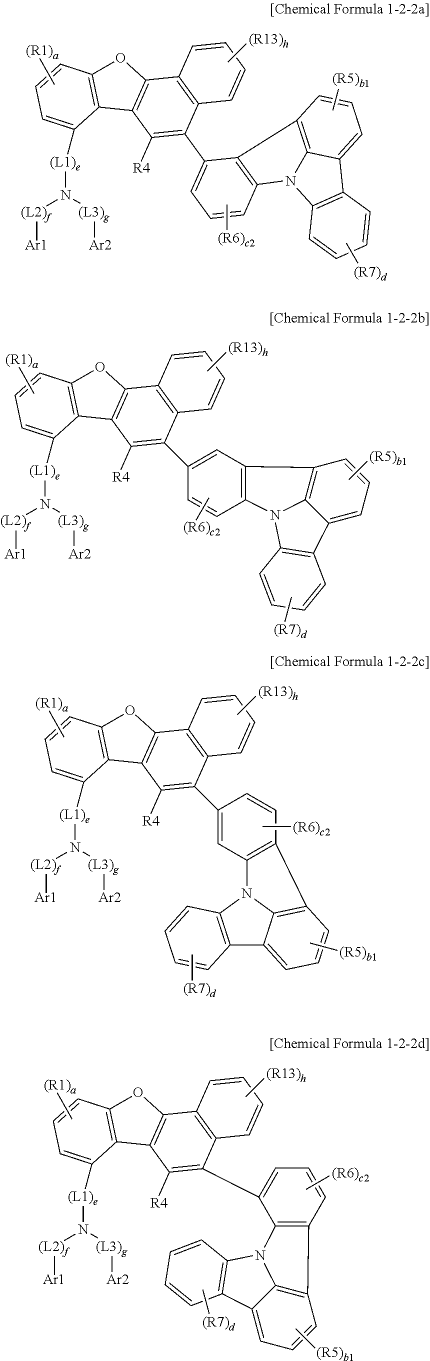

In Chemical Formulae 1-2-2a to 1-2-2d,
R1, R4 to R7, L1 to L3, Ar1, Ar2, a, b1 and d to g have the same definitions as in Chemical Formula 1,
R13 and h have the same definitions as in Chemical Formula 1-2, and
c2 has the same definition as in Chemical Formula 1-2-2.

In one embodiment of the present disclosure, R1 to R7, R11 to R13, L1 to L3, Ar1 and Ar2 may all include hydrogen (H) that is not deuterated.

In another embodiment of the present disclosure, at least one of R1 to R7, R11 to R13, L1 to L3, Ar1 and Ar2 includes deuterium (D), and at least one of R1 to R7, R11 to R13, L1 to L3, Ar1 and Ar2 may include hydrogen that is not deuterated.

In another embodiment of the present disclosure, R1 to R7, R1 to R13, L1 to L3, Ar1 and Ar2 may all include deuterium.

In one embodiment of the present disclosure, the heterocyclic compound represented by Chemical Formula 1 may not include deuterium as a substituent, or may have a deuterium content of, for example, greater than 0%, 1% or greater, 10% or greater, 20% or greater, 30% or greater, 40% or greater or 50% or greater, and 100% or less, 90% or less, 80% or less, 70% or less or 60% or less with respect to the total number of hydrogen atoms and deuterium atoms.

In another embodiment of the present disclosure, the heterocyclic compound represented by Chemical Formula 1 may not include deuterium as a substituent, or may have a deuterium content of 1% to 100% with respect to the total number of hydrogen atoms and deuterium atoms.

In another embodiment of the present disclosure, the heterocyclic compound represented by Chemical Formula 1 may not include deuterium as a substituent, or may have a deuterium content of 20% to 90% with respect to the total number of hydrogen atoms and deuterium atoms.

In another embodiment of the present disclosure, the heterocyclic compound represented by Chemical Formula 1 may not include deuterium as a substituent, or may have a deuterium content of 30% to 80% with respect to the total number of hydrogen atoms and deuterium atoms.

In another embodiment of the present disclosure, the heterocyclic compound represented by Chemical Formula 1 may not include deuterium as a substituent, or may have a deuterium content of 50% to 70% with respect to the total number of hydrogen atoms and deuterium atoms.

In one embodiment of the present disclosure, the heterocyclic compound represented by Chemical Formula 1 may be represented by any one of the following compounds.

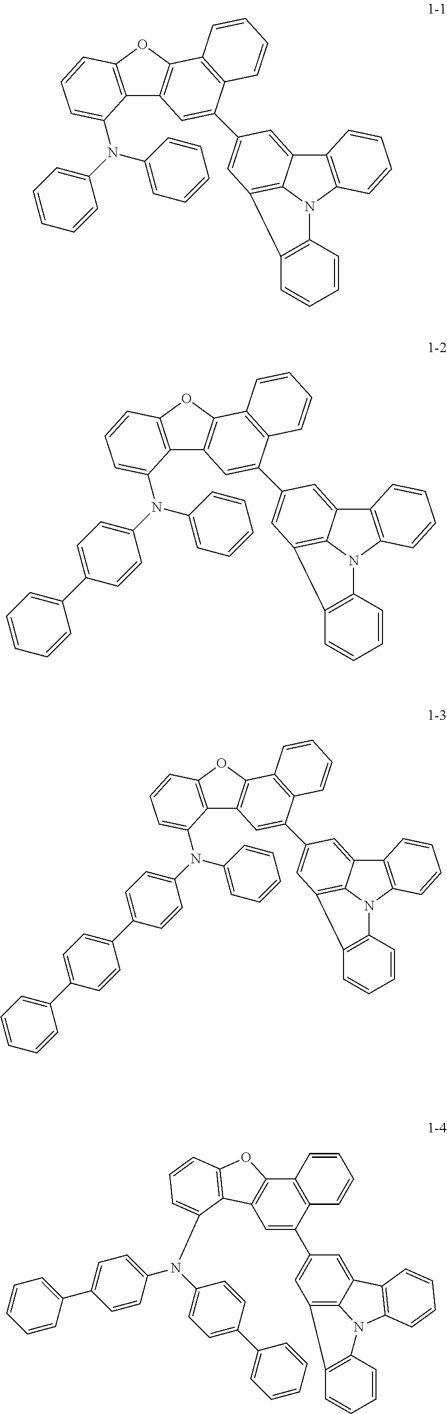 [SMILES: c1ccc(N(c2ccccc2)c2cccc3oc4c5ccccc5c(-c5cc6c7ccccc7n7c8ccccc8c(c5)c67)cc4c23)cc1 ; c1ccc(-c2ccc(N(c3ccccc3)c3cccc4oc5c6ccccc6c(-c6cc7c8ccccc8n8c9ccccc9c(c6)c78)cc5c34)cc2)cc1 ; c1ccc(-c2ccc(-c3ccc(N(c4ccccc4)c4cccc5oc6c7ccccc7c(-c7cc8c9ccccc9n9c%10ccccc%10c(c7)c89)cc6c45)cc3)cc2)cc1 ; c1ccc(-c2ccc(N(c3ccc(-c4ccccc4)cc3)c3cccc4oc5c6ccccc6c(-c6cc7c8ccccc8n8c9ccccc9c(c6)c78)cc5c34)cc2)cc1]

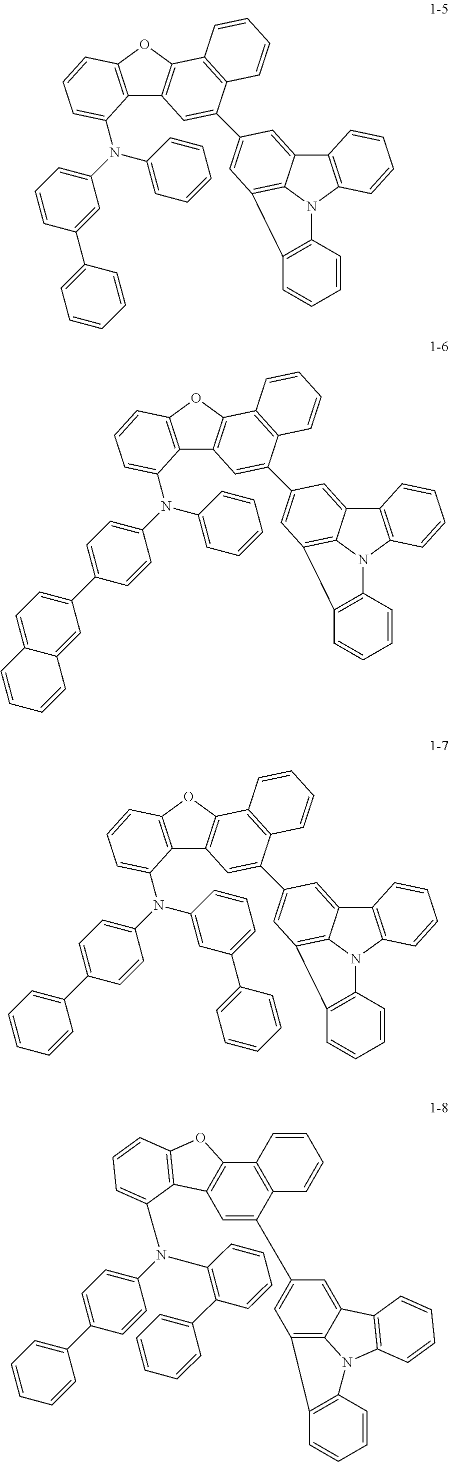 [SMILES: c1ccc(-c2cccc(N(c3ccccc3)c3cccc4oc5c6ccccc6c(-c6cc7c8ccccc8n8c9ccccc9c(c6)c78)cc5c34)c2)cc1 ; c1ccc(N(c2ccc(-c3ccc4ccccc4c3)cc2)c2cccc3oc4c5ccccc5c(-c5cc6c7ccccc7n7c8ccccc8c(c5)c67)cc4c23)cc1 ; c1ccc(-c2ccc(N(c3cccc(-c4ccccc4)c3)c3cccc4oc5c6ccccc6c(-c6cc7c8ccccc8n8c9ccccc9c(c6)c78)cc5c34)cc2)cc1 ; c1ccc(-c2ccc(N(c3ccccc3-c3ccccc3)c3cccc4oc5c6ccccc6c(-c6cc7c8ccccc8n8c9ccccc9c(c6)c78)cc5c34)cc2)cc1]

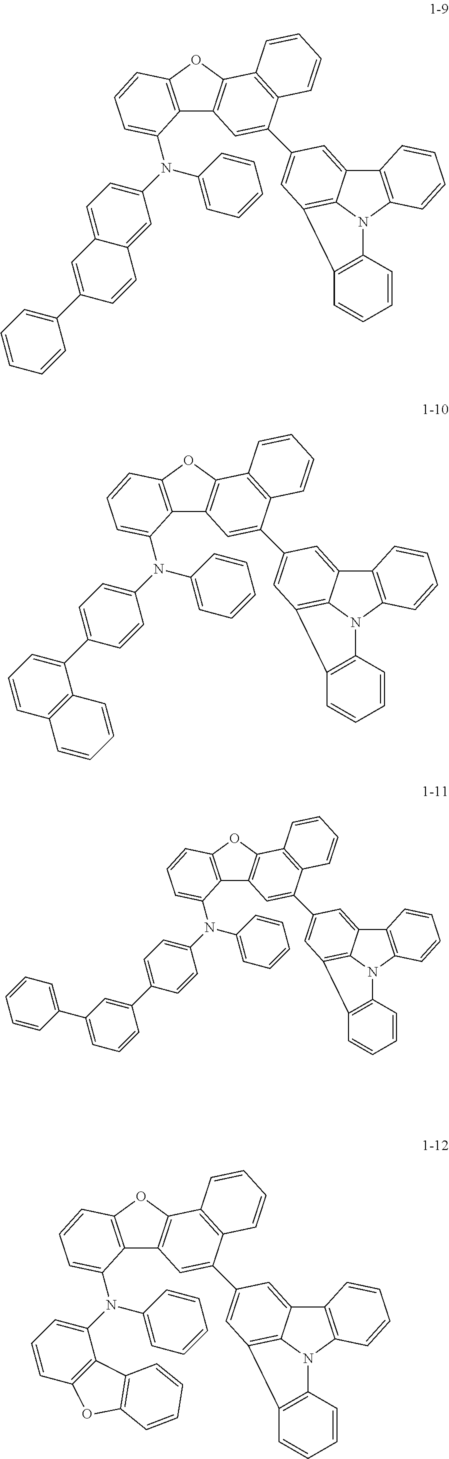 [SMILES: c1ccc(-c2ccc3cc(N(c4ccccc4)c4cccc5oc6c7ccccc7c(-c7cc8c9ccccc9n9c%10ccccc%10c(c7)c89)cc6c45)ccc3c2)cc1 ; c1ccc(N(c2ccc(-c3cccc4ccccc34)cc2)c2cccc3oc4c5ccccc5c(-c5cc6c7ccccc7n7c8ccccc8c(c5)c67)cc4c23)cc1 ; c1ccc(-c2cccc(-c3ccc(N(c4ccccc4)c4cccc5oc6c7ccccc7c(-c7cc8c9ccccc9n9c%10ccccc%10c(c7)c89)cc6c45)cc3)c2)cc1 ; c1ccc(N(c2cccc3oc4ccccc4c23)c2cccc3oc4c5ccccc5c(-c5cc6c7ccccc7n7c8ccccc8c(c5)c67)cc4c23)cc1]

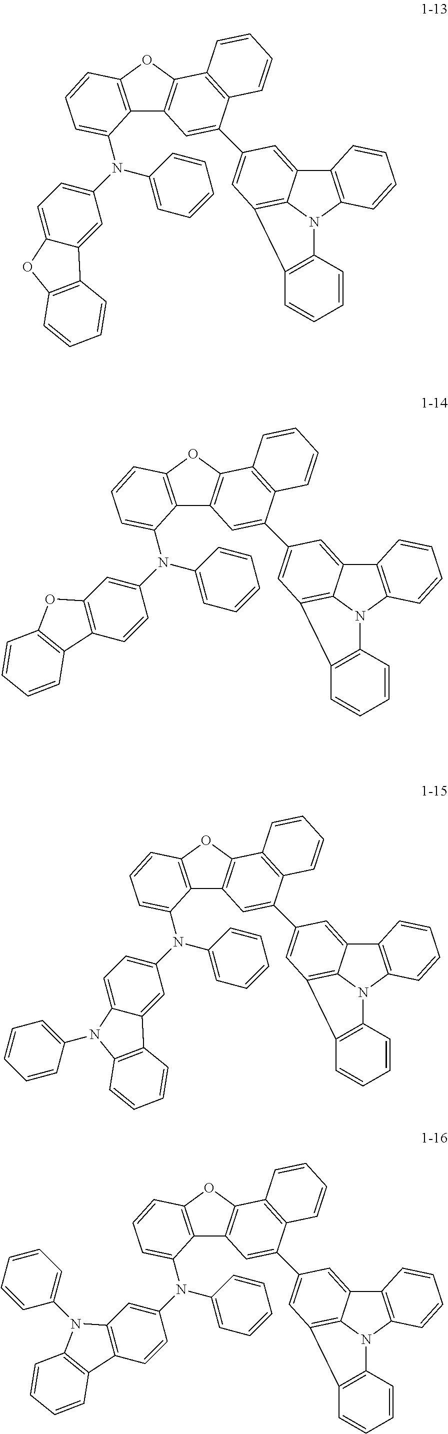 [SMILES: c1ccc(N(c2ccc3oc4ccccc4c3c2)c2cccc3oc4c5ccccc5c(-c5cc6c7ccccc7n7c8ccccc8c(c5)c67)cc4c23)cc1 ; c1ccc(N(c2ccc3c(c2)oc2ccccc23)c2cccc3oc4c5ccccc5c(-c5cc6c7ccccc7n7c8ccccc8c(c5)c67)cc4c23)cc1 ; c1ccc(N(c2ccc3c(c2)c2ccccc2n3-c2ccccc2)c2cccc3oc4c5ccccc5c(-c5cc6c7ccccc7n7c8ccccc8c(c5)c67)cc4c23)cc1 ; c1ccc(N(c2ccc3c4ccccc4n(-c4ccccc4)c3c2)c2cccc3oc4c5ccccc5c(-c5cc6c7ccccc7n7c8ccccc8c(c5)c67)cc4c23)cc1]

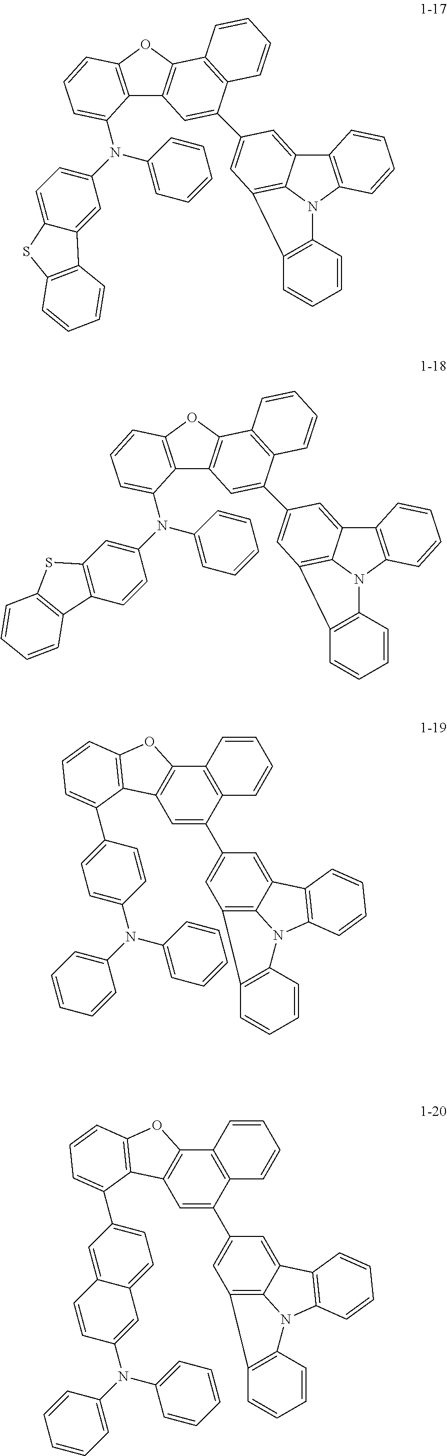 [SMILES: c1ccc(N(c2ccc3sc4ccccc4c3c2)c2cccc3oc4c5ccccc5c(-c5cc6c7ccccc7n7c8ccccc8c(c5)c67)cc4c23)cc1 ; c1ccc(N(c2ccc3c(c2)sc2ccccc23)c2cccc3oc4c5ccccc5c(-c5cc6c7ccccc7n7c8ccccc8c(c5)c67)cc4c23)cc1 ; c1ccc(N(c2ccccc2)c2ccc(-c3cccc4oc5c6ccccc6c(-c6cc7c8ccccc8n8c9ccccc9c(c6)c78)cc5c34)cc2)cc1 ; c1ccc(N(c2ccccc2)c2ccc3cc(-c4cccc5oc6c7ccccc7c(-c7cc8c9ccccc9n9c%10ccccc%10c(c7)c89)cc6c45)ccc3c2)cc1]

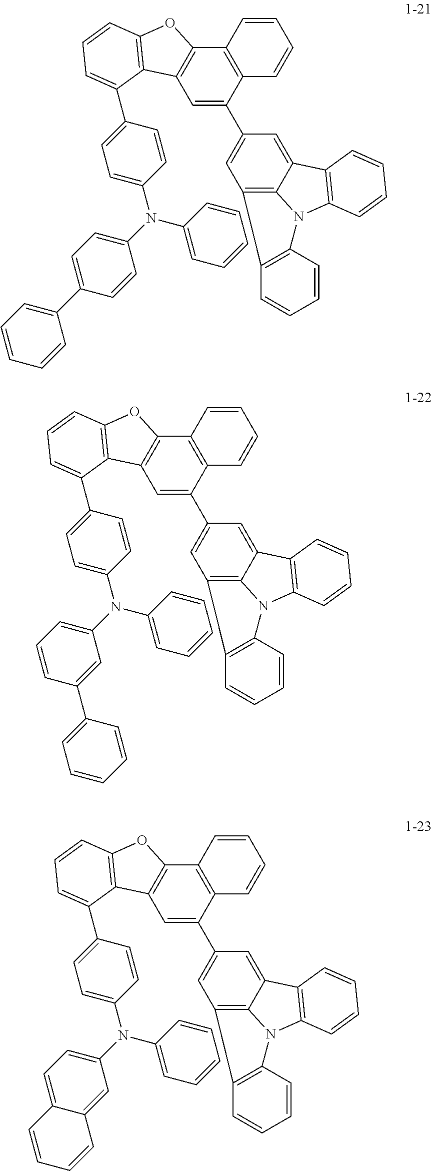 [SMILES: c1ccc(-c2ccc(N(c3ccccc3)c3ccc(-c4cccc5oc6c7ccccc7c(-c7cc8c9ccccc9n9c%10ccccc%10c(c7)c89)cc6c45)cc3)cc2)cc1 ; c1ccc(-c2cccc(N(c3ccccc3)c3ccc(-c4cccc5oc6c7ccccc7c(-c7cc8c9ccccc9n9c%10ccccc%10c(c7)c89)cc6c45)cc3)c2)cc1 ; c1ccc(N(c2ccc(-c3cccc4oc5c6ccccc6c(-c6cc7c8ccccc8n8c9ccccc9c(c6)c78)cc5c34)cc2)c2ccc3ccccc3c2)cc1]

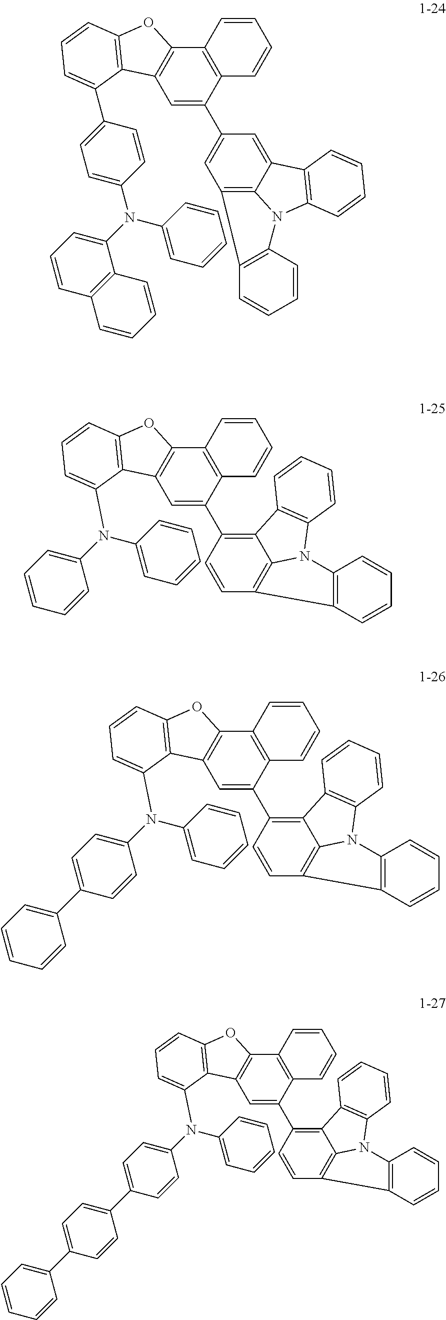 [SMILES: c1ccc(N(c2ccc(-c3cccc4oc5c6ccccc6c(-c6cc7c8ccccc8n8c9ccccc9c(c6)c78)cc5c34)cc2)c2cccc3ccccc23)cc1 ; c1ccc(N(c2ccccc2)c2cccc3oc4c5ccccc5c(-c5ccc6c7ccccc7n7c8ccccc8c5c67)cc4c23)cc1 ; c1ccc(-c2ccc(N(c3ccccc3)c3cccc4oc5c6ccccc6c(-c6ccc7c8ccccc8n8c9ccccc9c6c78)cc5c34)cc2)cc1 ; c1ccc(-c2ccc(-c3ccc(N(c4ccccc4)c4cccc5oc6c7ccccc7c(-c7ccc8c9ccccc9n9c%10ccccc%10c7c89)cc6c45)cc3)cc2)cc1]

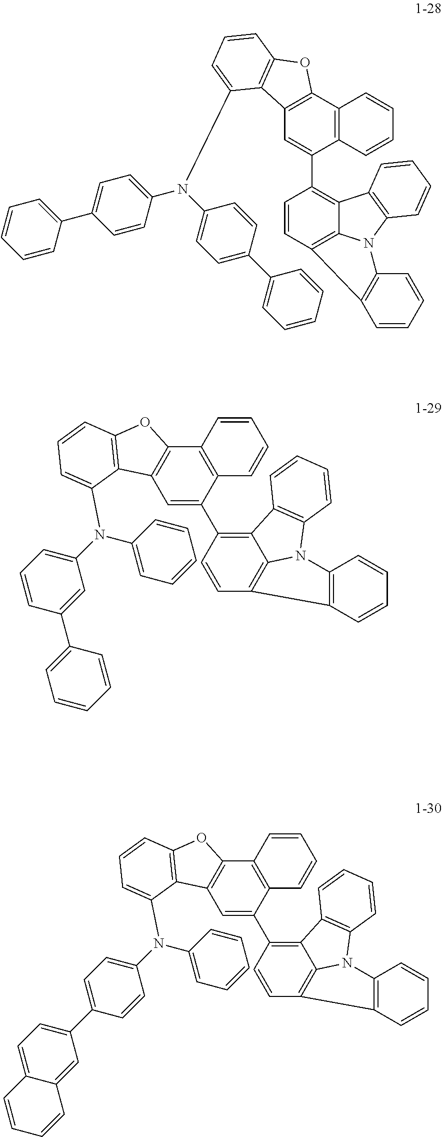 [SMILES: c1ccc(-c2ccc(N(c3ccc(-c4ccccc4)cc3)c3cccc4oc5c6ccccc6c(-c6ccc7c8ccccc8n8c9ccccc9c6c78)cc5c34)cc2)cc1 ; c1ccc(-c2cccc(N(c3ccccc3)c3cccc4oc5c6ccccc6c(-c6ccc7c8ccccc8n8c9ccccc9c6c78)cc5c34)c2)cc1 ; c1ccc(N(c2ccc(-c3ccc4ccccc4c3)cc2)c2cccc3oc4c5ccccc5c(-c5ccc6c7ccccc7n7c8ccccc8c5c67)cc4c23)cc1]

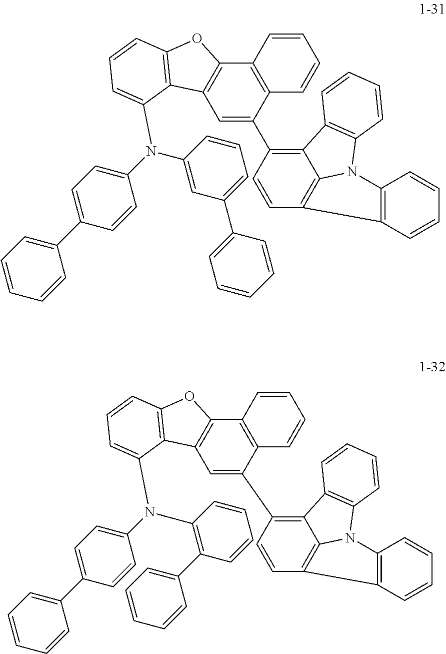 [SMILES: c1ccc(-c2ccc(N(c3cccc(-c4ccccc4)c3)c3cccc4oc5c6ccccc6c(-c6ccc7c8ccccc8n8c9ccccc9c6c78)cc5c34)cc2)cc1 ; c1ccc(-c2ccc(N(c3ccccc3-c3ccccc3)c3cccc4oc5c6ccccc6c(-c6ccc7c8ccccc8n8c9ccccc9c6c78)cc5c34)cc2)cc1]

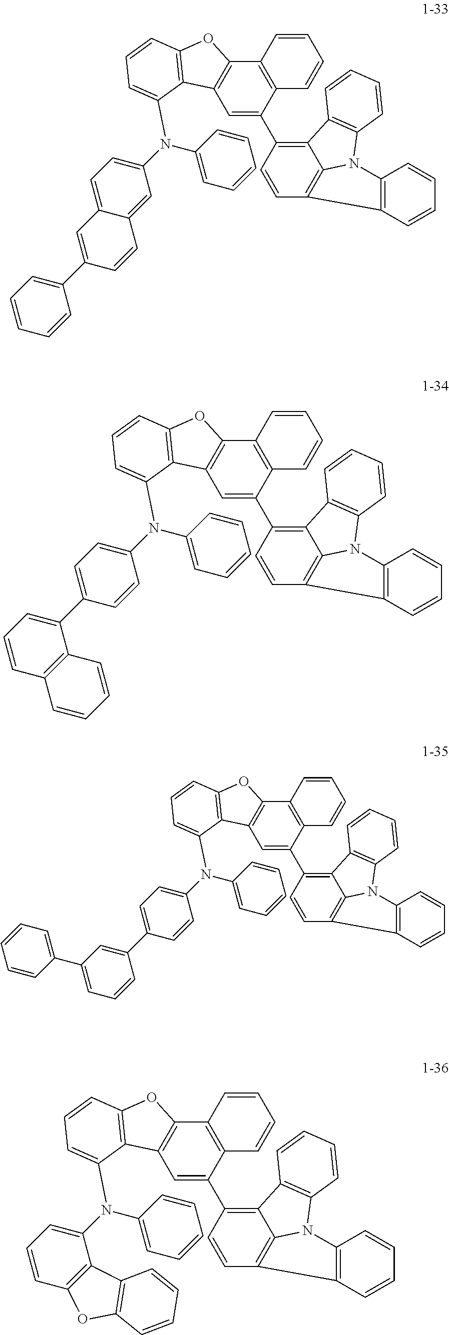 [SMILES: c1ccc(-c2ccc3cc(N(c4ccccc4)c4cccc5oc6c7ccccc7c(-c7ccc8c9ccccc9n9c%10ccccc%10c7c89)cc6c45)ccc3c2)cc1 ; c1ccc(N(c2ccc(-c3cccc4ccccc34)cc2)c2cccc3oc4c5ccccc5c(-c5ccc6c7ccccc7n7c8ccccc8c5c67)cc4c23)cc1 ; c1ccc(-c2cccc(-c3ccc(N(c4ccccc4)c4cccc5oc6c7ccccc7c(-c7ccc8c9ccccc9n9c%10ccccc%10c7c89)cc6c45)cc3)c2)cc1 ; c1ccc(N(c2cccc3oc4ccccc4c23)c2cccc3oc4c5ccccc5c(-c5ccc6c7ccccc7n7c8ccccc8c5c67)cc4c23)cc1]

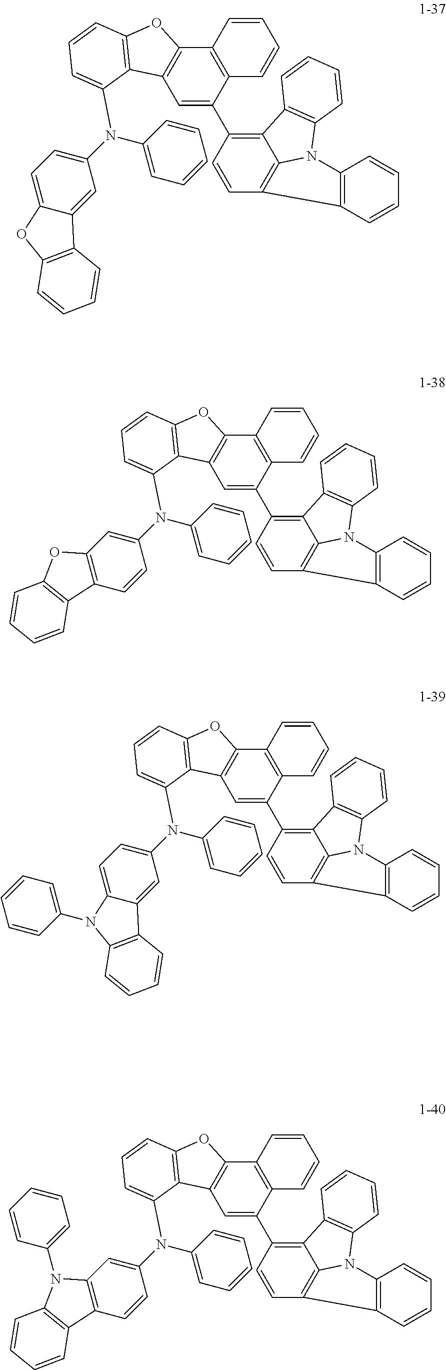 [SMILES: c1ccc(N(c2ccc3oc4ccccc4c3c2)c2cccc3oc4c5ccccc5c(-c5ccc6c7ccccc7n7c8ccccc8c5c67)cc4c23)cc1 ; c1ccc(N(c2ccc3c(c2)oc2ccccc23)c2cccc3oc4c5ccccc5c(-c5ccc6c7ccccc7n7c8ccccc8c5c67)cc4c23)cc1 ; c1ccc(N(c2ccc3c(c2)c2ccccc2n3-c2ccccc2)c2cccc3oc4c5ccccc5c(-c5ccc6c7ccccc7n7c8ccccc8c5c67)cc4c23)cc1 ; c1ccc(N(c2ccc3c4ccccc4n(-c4ccccc4)c3c2)c2cccc3oc4c5ccccc5c(-c5ccc6c7ccccc7n7c8ccccc8c5c67)cc4c23)cc1]

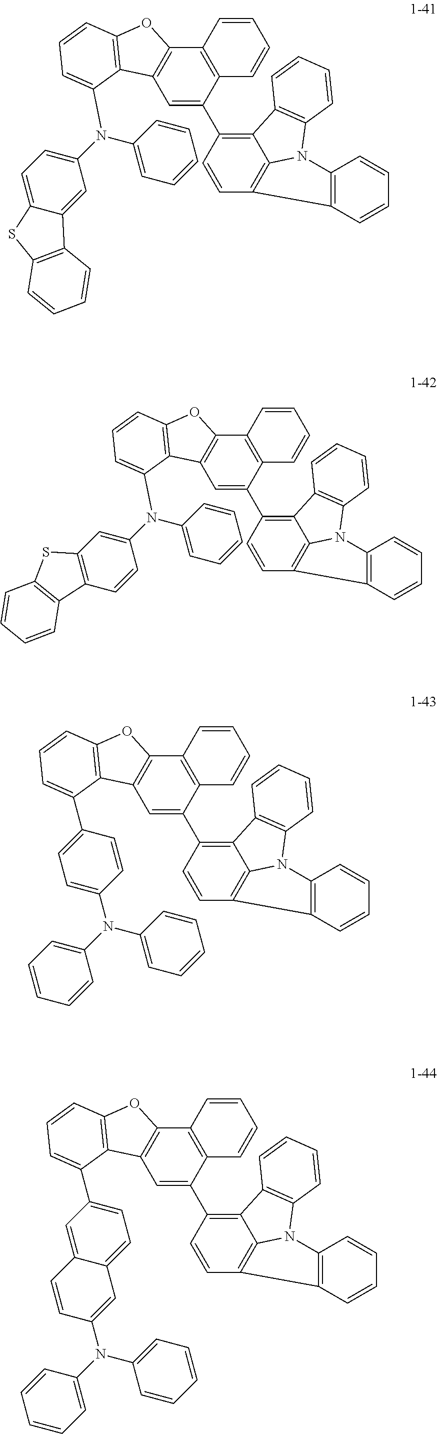 [SMILES: c1ccc(N(c2ccc3sc4ccccc4c3c2)c2cccc3oc4c5ccccc5c(-c5ccc6c7ccccc7n7c8ccccc8c5c67)cc4c23)cc1 ; c1ccc(N(c2ccc3c(c2)sc2ccccc23)c2cccc3oc4c5ccccc5c(-c5ccc6c7ccccc7n7c8ccccc8c5c67)cc4c23)cc1 ; c1ccc(N(c2ccccc2)c2ccc(-c3cccc4oc5c6ccccc6c(-c6ccc7c8ccccc8n8c9ccccc9c6c78)cc5c34)cc2)cc1 ; c1ccc(N(c2ccccc2)c2ccc3cc(-c4cccc5oc6c7ccccc7c(-c7ccc8c9ccccc9n9c%10ccccc%10c7c89)cc6c45)ccc3c2)cc1]

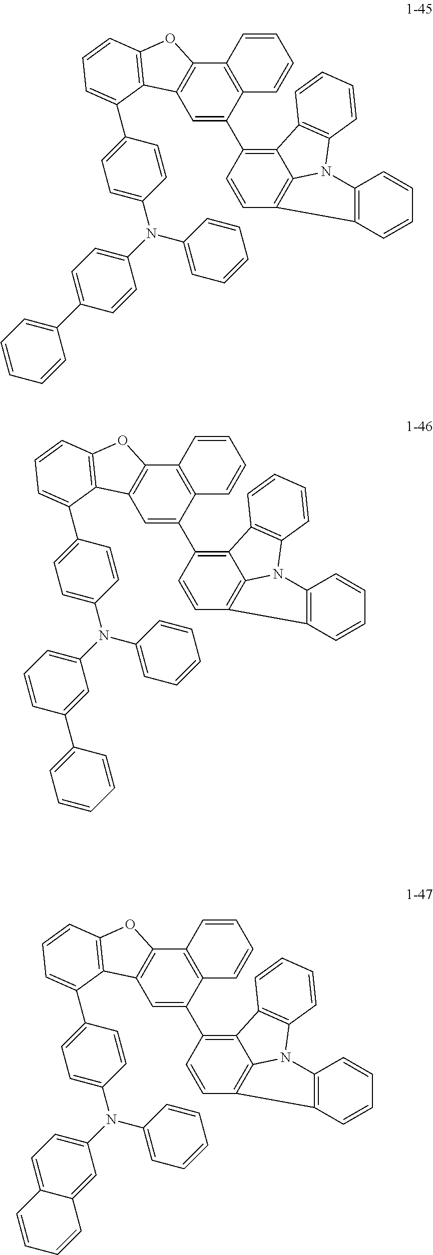 [SMILES: c1ccc(-c2ccc(N(c3ccccc3)c3ccc(-c4cccc5oc6c7ccccc7c(-c7ccc8c9ccccc9n9c%10ccccc%10c7c89)cc6c45)cc3)cc2)cc1 ; c1ccc(-c2cccc(N(c3ccccc3)c3ccc(-c4cccc5oc6c7ccccc7c(-c7ccc8c9ccccc9n9c%10ccccc%10c7c89)cc6c45)cc3)c2)cc1 ; c1ccc(N(c2ccc(-c3cccc4oc5c6ccccc6c(-c6ccc7c8ccccc8n8c9ccccc9c6c78)cc5c34)cc2)c2ccc3ccccc3c2)cc1]

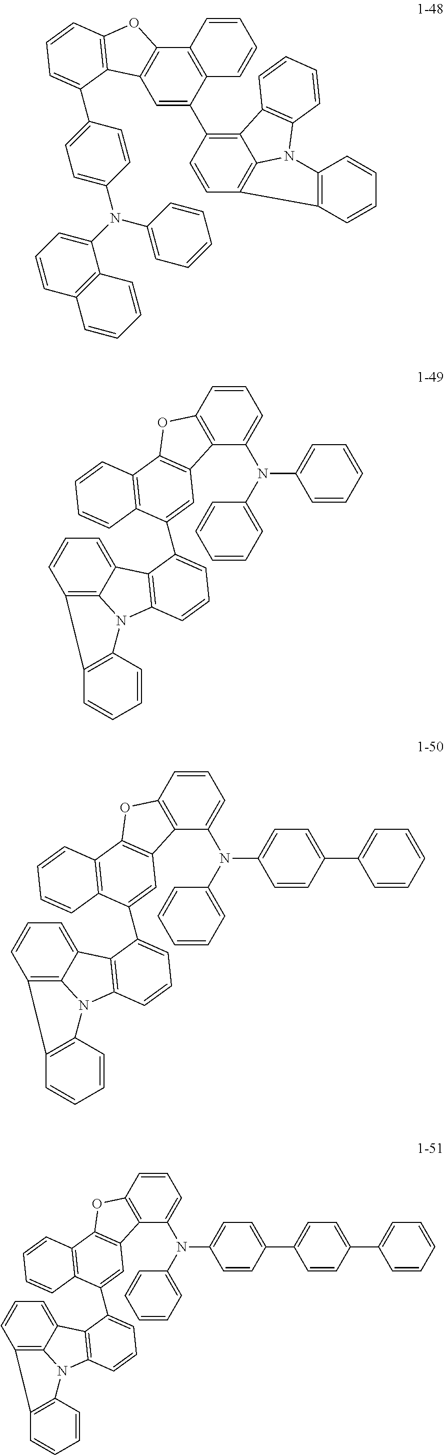 [SMILES: c1ccc(N(c2ccc(-c3cccc4oc5c6ccccc6c(-c6ccc7c8ccccc8n8c9ccccc9c6c78)cc5c34)cc2)c2cccc3ccccc23)cc1 ; c1ccc(N(c2ccccc2)c2cccc3oc4c5ccccc5c(-c5cccc6c5c5cccc7c8ccccc8n6c75)cc4c23)cc1 ; c1ccc(-c2ccc(N(c3ccccc3)c3cccc4oc5c6ccccc6c(-c6cccc7c6c6cccc8c9ccccc9n7c86)cc5c34)cc2)cc1 ; c1ccc(-c2ccc(-c3ccc(N(c4ccccc4)c4cccc5oc6c7ccccc7c(-c7cccc8c7c7cccc9c%10ccccc%10n8c97)cc6c45)cc3)cc2)cc1]

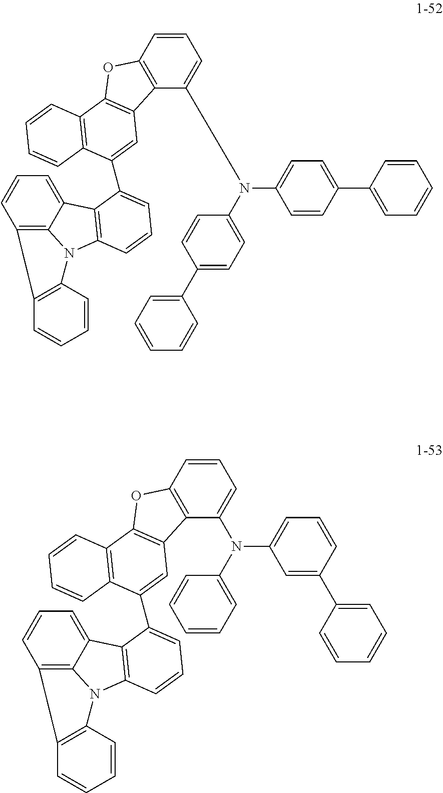 [SMILES: c1ccc(-c2ccc(N(c3ccc(-c4ccccc4)cc3)c3cccc4oc5c6ccccc6c(-c6cccc7c6c6cccc8c9ccccc9n7c86)cc5c34)cc2)cc1 ; c1ccc(-c2cccc(N(c3ccccc3)c3cccc4oc5c6ccccc6c(-c6cccc7c6c6cccc8c9ccccc9n7c86)cc5c34)c2)cc1]

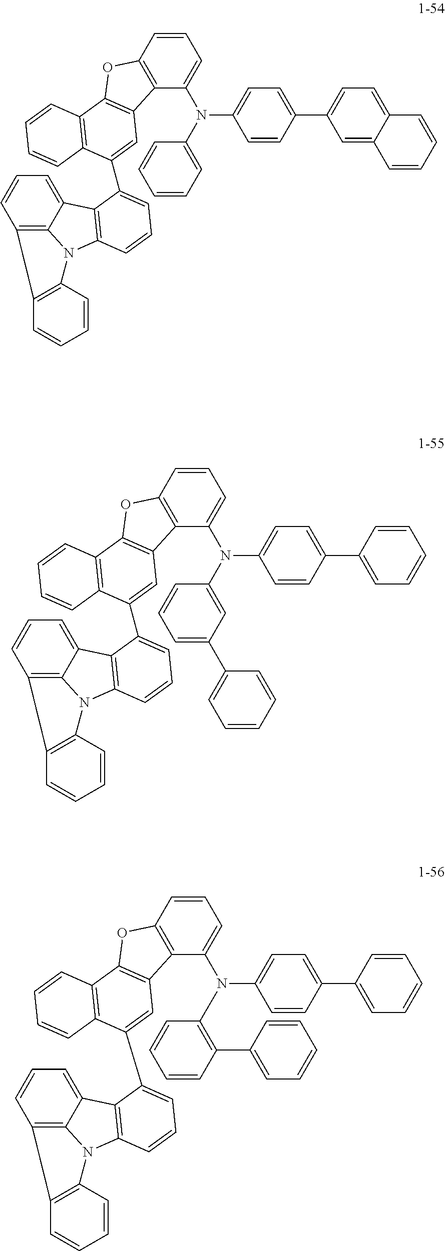 [SMILES: c1ccc(N(c2ccc(-c3ccc4ccccc4c3)cc2)c2cccc3oc4c5ccccc5c(-c5cccc6c5c5cccc7c8ccccc8n6c75)cc4c23)cc1 ; c1ccc(-c2ccc(N(c3cccc(-c4ccccc4)c3)c3cccc4oc5c6ccccc6c(-c6cccc7c6c6cccc8c9ccccc9n7c86)cc5c34)cc2)cc1 ; c1ccc(-c2ccc(N(c3ccccc3-c3ccccc3)c3cccc4oc5c6ccccc6c(-c6cccc7c6c6cccc8c9ccccc9n7c86)cc5c34)cc2)cc1]

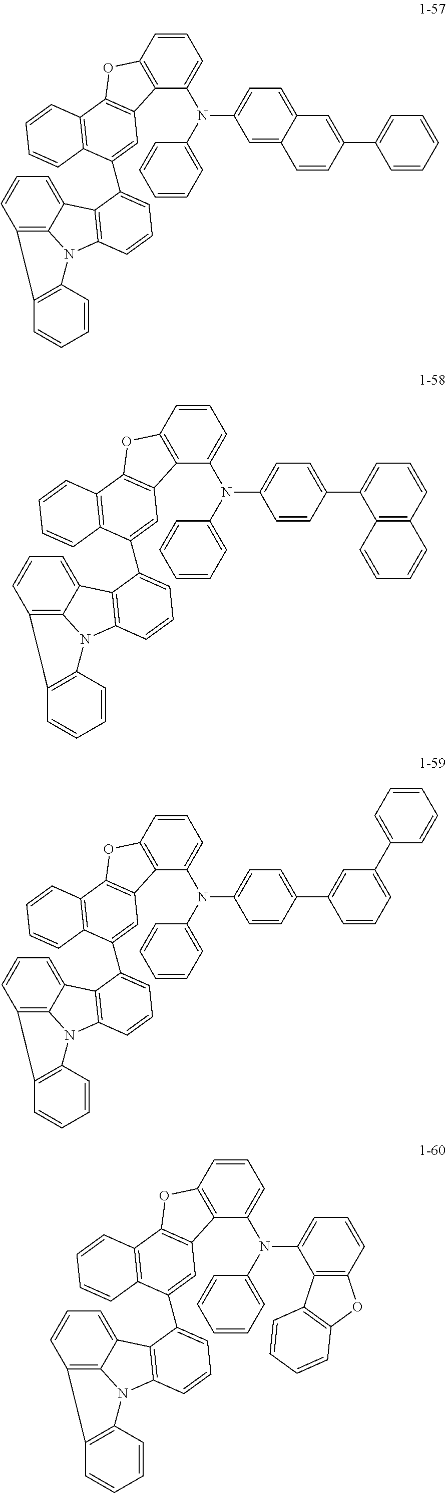 [SMILES: c1ccc(-c2ccc3cc(N(c4ccccc4)c4cccc5oc6c7ccccc7c(-c7cccc8c7c7cccc9c%10ccccc%10n8c97)cc6c45)ccc3c2)cc1 ; c1ccc(N(c2ccc(-c3cccc4ccccc34)cc2)c2cccc3oc4c5ccccc5c(-c5cccc6c5c5cccc7c8ccccc8n6c75)cc4c23)cc1 ; c1ccc(-c2cccc(-c3ccc(N(c4ccccc4)c4cccc5oc6c7ccccc7c(-c7cccc8c7c7cccc9c%10ccccc%10n8c97)cc6c45)cc3)c2)cc1 ; c1ccc(N(c2cccc3oc4ccccc4c23)c2cccc3oc4c5ccccc5c(-c5cccc6c5c5cccc7c8ccccc8n6c75)cc4c23)cc1]

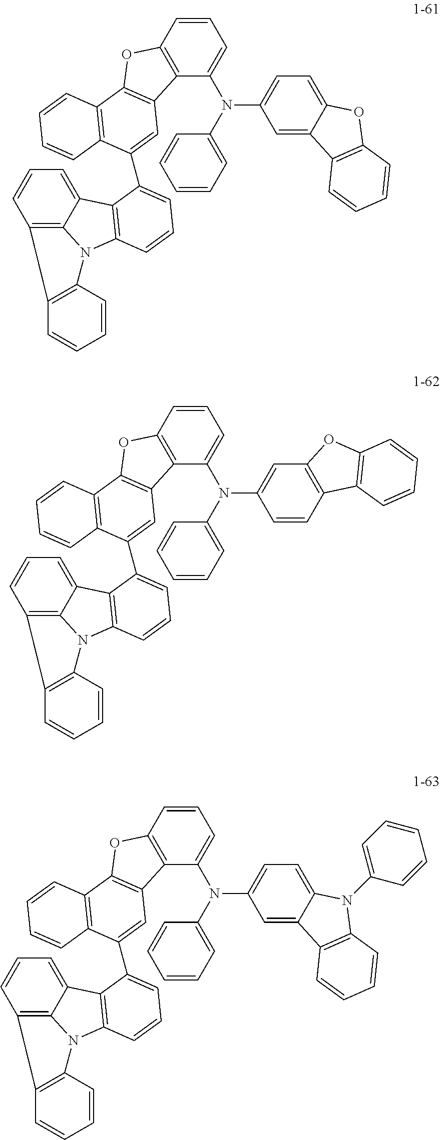 [SMILES: c1ccc(N(c2ccc3oc4ccccc4c3c2)c2cccc3oc4c5ccccc5c(-c5cccc6c5c5cccc7c8ccccc8n6c75)cc4c23)cc1 ; c1ccc(N(c2ccc3c(c2)oc2ccccc23)c2cccc3oc4c5ccccc5c(-c5cccc6c5c5cccc7c8ccccc8n6c75)cc4c23)cc1 ; c1ccc(N(c2ccc3c(c2)c2ccccc2n3-c2ccccc2)c2cccc3oc4c5ccccc5c(-c5cccc6c5c5cccc7c8ccccc8n6c75)cc4c23)cc1]

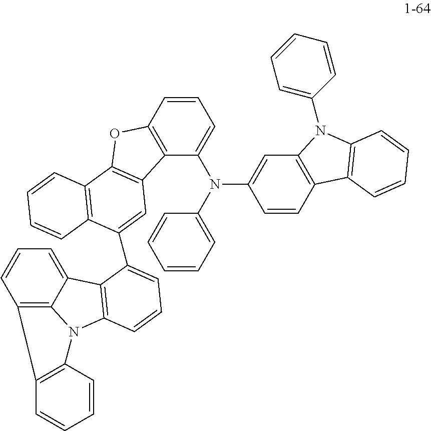 [SMILES: c1ccc(N(c2ccc3c4ccccc4n(-c4ccccc4)c3c2)c2cccc3oc4c5ccccc5c(-c5cccc6c5c5cccc7c8ccccc8n6c75)cc4c23)cc1]

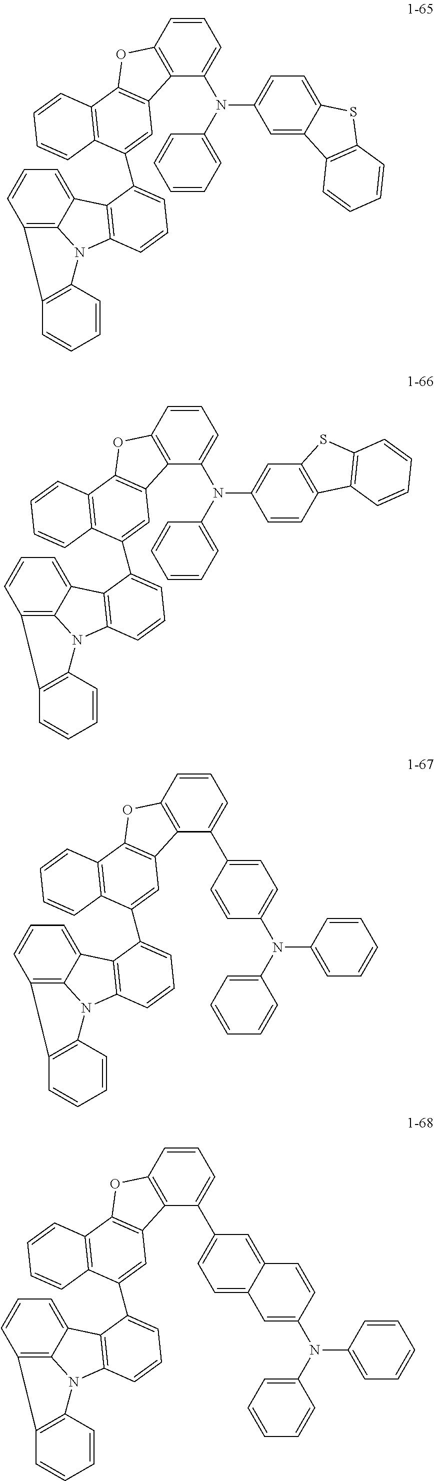 [SMILES: c1ccc(N(c2ccc3sc4ccccc4c3c2)c2cccc3oc4c5ccccc5c(-c5cccc6c5c5cccc7c8ccccc8n6c75)cc4c23)cc1 ; c1ccc(N(c2ccc3c(c2)sc2ccccc23)c2cccc3oc4c5ccccc5c(-c5cccc6c5c5cccc7c8ccccc8n6c75)cc4c23)cc1 ; c1ccc(N(c2ccccc2)c2ccc(-c3cccc4oc5c6ccccc6c(-c6cccc7c6c6cccc8c9ccccc9n7c86)cc5c34)cc2)cc1 ; c1ccc(N(c2ccccc2)c2ccc3cc(-c4cccc5oc6c7ccccc7c(-c7cccc8c7c7cccc9c%10ccccc%10n8c97)cc6c45)ccc3c2)cc1]

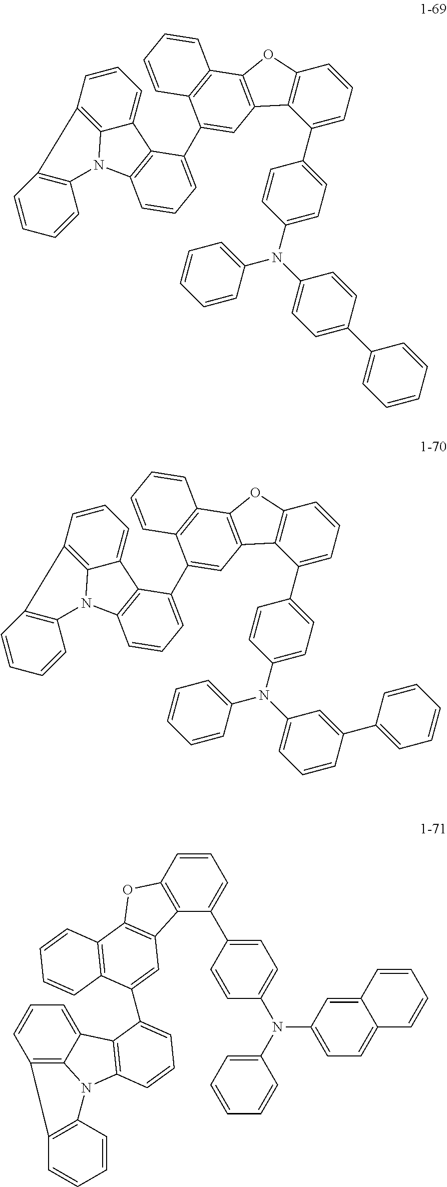 [SMILES: c1ccc(-c2ccc(N(c3ccccc3)c3ccc(-c4cccc5oc6c7ccccc7c(-c7cccc8c7c7cccc9c%10ccccc%10n8c97)cc6c45)cc3)cc2)cc1 ; c1ccc(-c2cccc(N(c3ccccc3)c3ccc(-c4cccc5oc6c7ccccc7c(-c7cccc8c7c7cccc9c%10ccccc%10n8c97)cc6c45)cc3)c2)cc1 ; c1ccc(N(c2ccc(-c3cccc4oc5c6ccccc6c(-c6cccc7c6c6cccc8c9ccccc9n7c86)cc5c34)cc2)c2ccc3ccccc3c2)cc1]

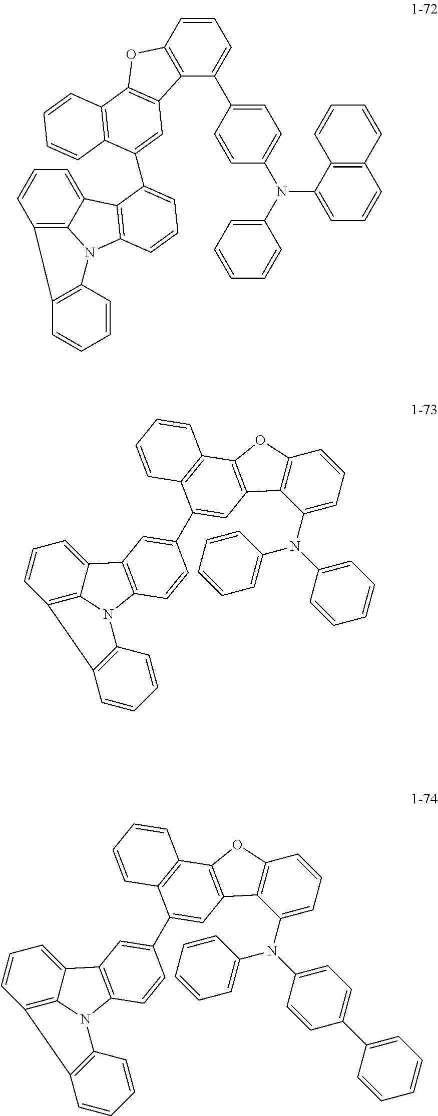 [SMILES: c1ccc(N(c2ccc(-c3cccc4oc5c6ccccc6c(-c6cccc7c6c6cccc8c9ccccc9n7c86)cc5c34)cc2)c2cccc3ccccc23)cc1 ; c1ccc(N(c2ccccc2)c2cccc3oc4c5ccccc5c(-c5ccc6c(c5)c5cccc7c8ccccc8n6c75)cc4c23)cc1 ; c1ccc(-c2ccc(N(c3ccccc3)c3cccc4oc5c6ccccc6c(-c6ccc7c(c6)c6cccc8c9ccccc9n7c86)cc5c34)cc2)cc1]

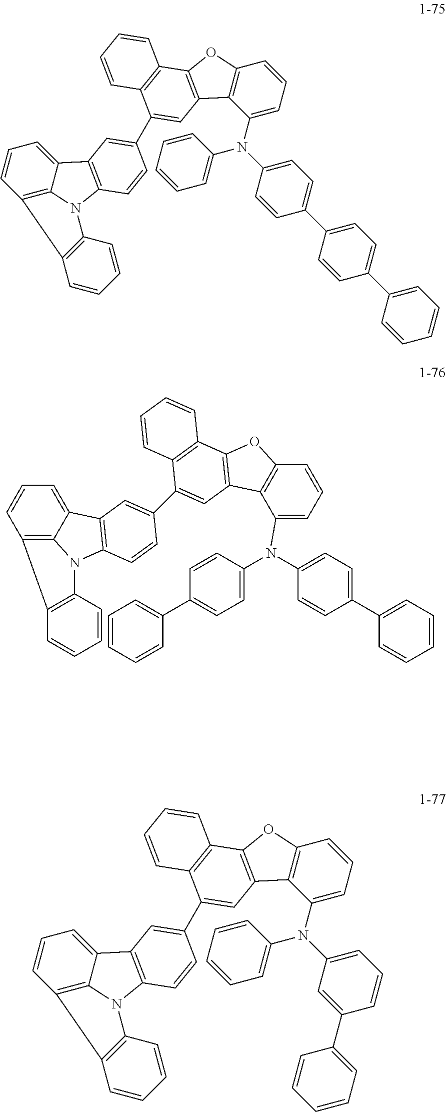 [SMILES: c1ccc(-c2ccc(-c3ccc(N(c4ccccc4)c4cccc5oc6c7ccccc7c(-c7ccc8c(c7)c7cccc9c%10ccccc%10n8c97)cc6c45)cc3)cc2)cc1 ; c1ccc(-c2ccc(N(c3ccc(-c4ccccc4)cc3)c3cccc4oc5c6ccccc6c(-c6ccc7c(c6)c6cccc8c9ccccc9n7c86)cc5c34)cc2)cc1 ; c1ccc(-c2cccc(N(c3ccccc3)c3cccc4oc5c6ccccc6c(-c6ccc7c(c6)c6cccc8c9ccccc9n7c86)cc5c34)c2)cc1]

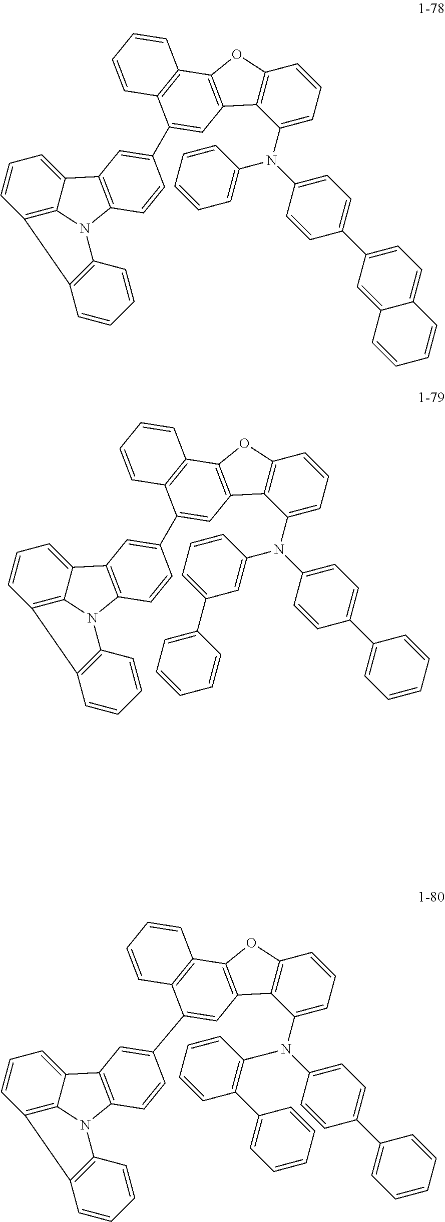 [SMILES: c1ccc(N(c2ccc(-c3ccc4ccccc4c3)cc2)c2cccc3oc4c5ccccc5c(-c5ccc6c(c5)c5cccc7c8ccccc8n6c75)cc4c23)cc1 ; c1ccc(-c2ccc(N(c3cccc(-c4ccccc4)c3)c3cccc4oc5c6ccccc6c(-c6ccc7c(c6)c6cccc8c9ccccc9n7c86)cc5c34)cc2)cc1 ; c1ccc(-c2ccc(N(c3ccccc3-c3ccccc3)c3cccc4oc5c6ccccc6c(-c6ccc7c(c6)c6cccc8c9ccccc9n7c86)cc5c34)cc2)cc1]

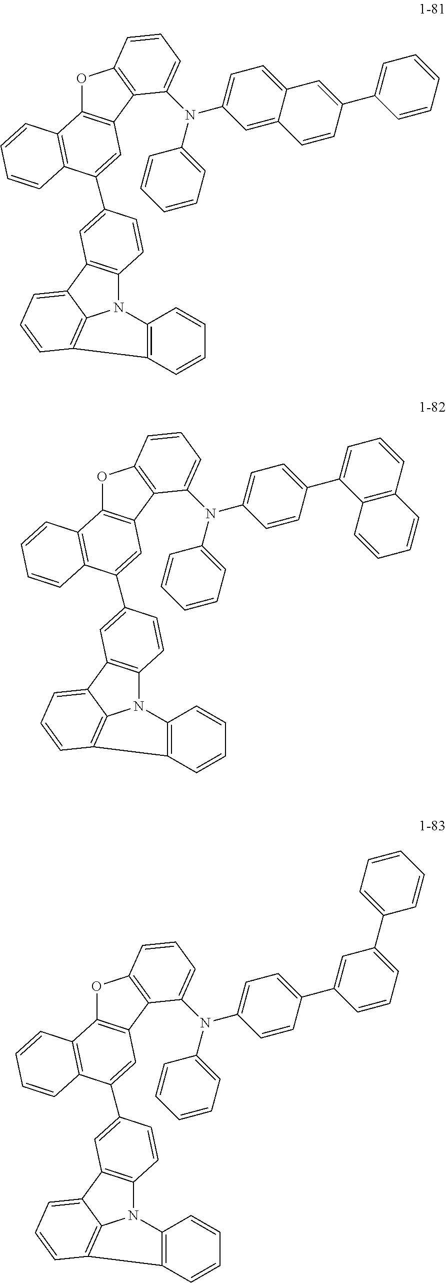 [SMILES: c1ccc(-c2ccc3cc(N(c4ccccc4)c4cccc5oc6c7ccccc7c(-c7ccc8c(c7)c7cccc9c%10ccccc%10n8c97)cc6c45)ccc3c2)cc1 ; c1ccc(N(c2ccc(-c3cccc4ccccc34)cc2)c2cccc3oc4c5ccccc5c(-c5ccc6c(c5)c5cccc7c8ccccc8n6c75)cc4c23)cc1 ; c1ccc(-c2cccc(-c3ccc(N(c4ccccc4)c4cccc5oc6c7ccccc7c(-c7ccc8c(c7)c7cccc9c%10ccccc%10n8c97)cc6c45)cc3)c2)cc1]

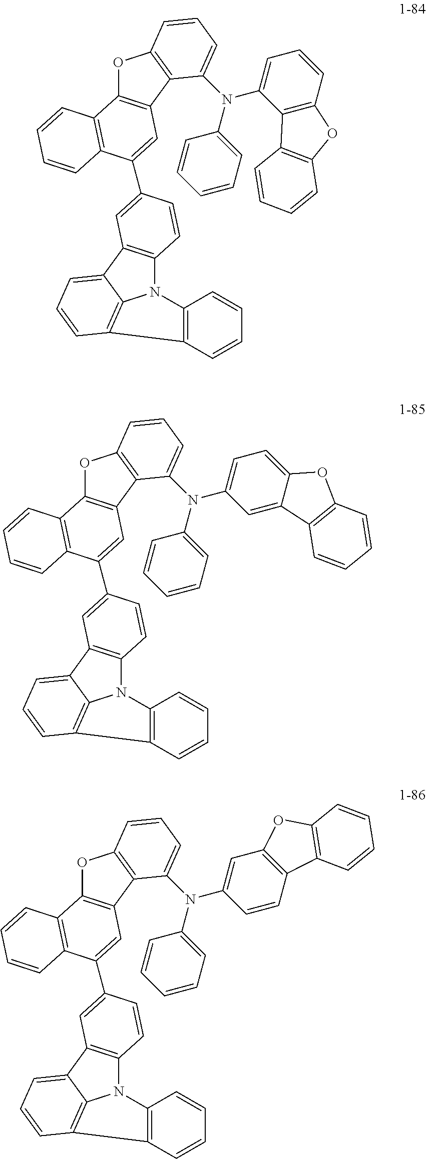 [SMILES: c1ccc(N(c2cccc3oc4ccccc4c23)c2cccc3oc4c5ccccc5c(-c5ccc6c(c5)c5cccc7c8ccccc8n6c75)cc4c23)cc1 ; c1ccc(N(c2ccc3oc4ccccc4c3c2)c2cccc3oc4c5ccccc5c(-c5ccc6c(c5)c5cccc7c8ccccc8n6c75)cc4c23)cc1 ; c1ccc(N(c2ccc3c(c2)oc2ccccc23)c2cccc3oc4c5ccccc5c(-c5ccc6c(c5)c5cccc7c8ccccc8n6c75)cc4c23)cc1]

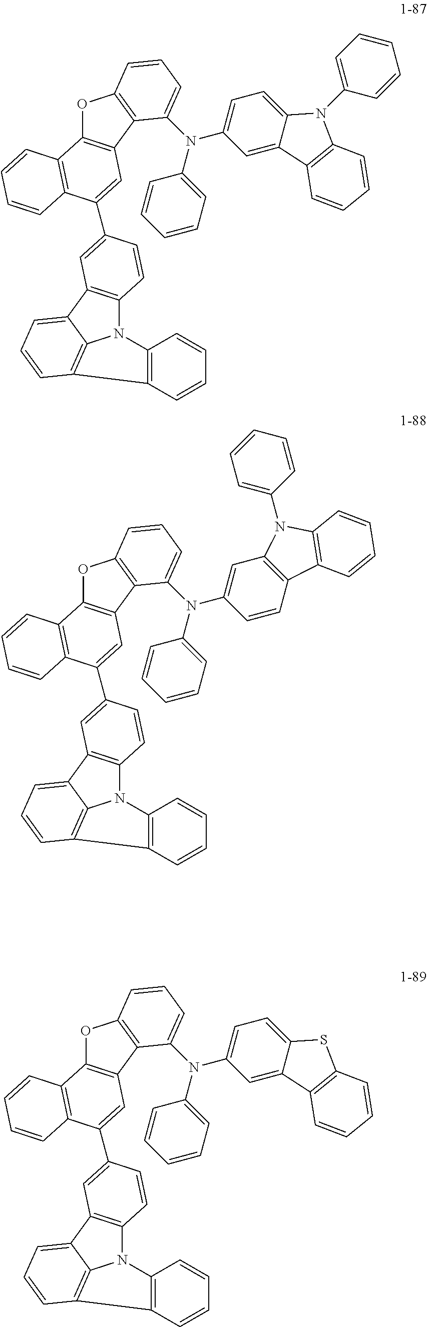 [SMILES: c1ccc(N(c2ccc3c(c2)c2ccccc2n3-c2ccccc2)c2cccc3oc4c5ccccc5c(-c5ccc6c(c5)c5cccc7c8ccccc8n6c75)cc4c23)cc1 ; c1ccc(N(c2ccc3c4ccccc4n(-c4ccccc4)c3c2)c2cccc3oc4c5ccccc5c(-c5ccc6c(c5)c5cccc7c8ccccc8n6c75)cc4c23)cc1 ; c1ccc(N(c2ccc3sc4ccccc4c3c2)c2cccc3oc4c5ccccc5c(-c5ccc6c(c5)c5cccc7c8ccccc8n6c75)cc4c23)cc1]

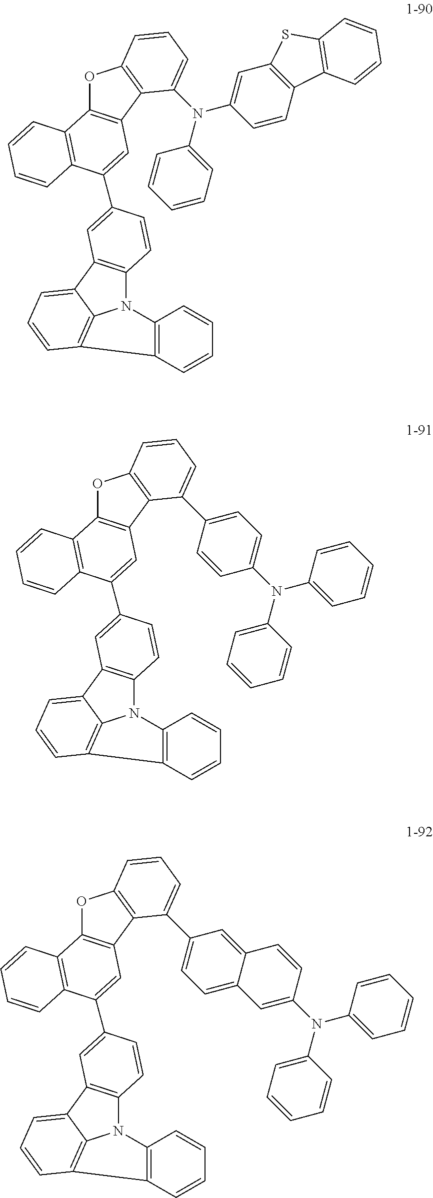 [SMILES: c1ccc(N(c2ccc3c(c2)sc2ccccc23)c2cccc3oc4c5ccccc5c(-c5ccc6c(c5)c5cccc7c8ccccc8n6c75)cc4c23)cc1 ; c1ccc(N(c2ccccc2)c2ccc(-c3cccc4oc5c6ccccc6c(-c6ccc7c(c6)c6cccc8c9ccccc9n7c86)cc5c34)cc2)cc1 ; c1ccc(N(c2ccccc2)c2ccc3cc(-c4cccc5oc6c7ccccc7c(-c7ccc8c(c7)c7cccc9c%10ccccc%10n8c97)cc6c45)ccc3c2)cc1]

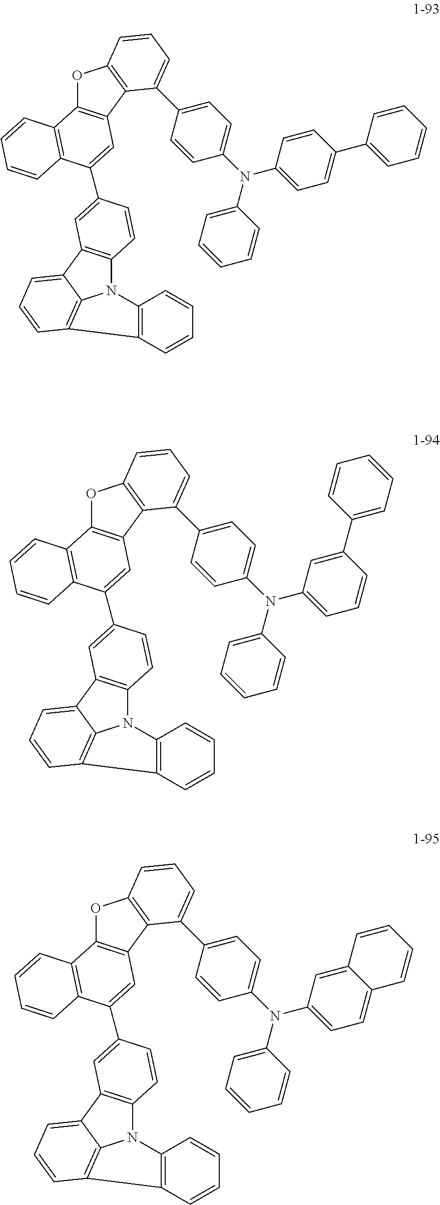 [SMILES: c1ccc(-c2ccc(N(c3ccccc3)c3ccc(-c4cccc5oc6c7ccccc7c(-c7ccc8c(c7)c7cccc9c%10ccccc%10n8c97)cc6c45)cc3)cc2)cc1 ; c1ccc(-c2cccc(N(c3ccccc3)c3ccc(-c4cccc5oc6c7ccccc7c(-c7ccc8c(c7)c7cccc9c%10ccccc%10n8c97)cc6c45)cc3)c2)cc1 ; c1ccc(N(c2ccc(-c3cccc4oc5c6ccccc6c(-c6ccc7c(c6)c6cccc8c9ccccc9n7c86)cc5c34)cc2)c2ccc3ccccc3c2)cc1]

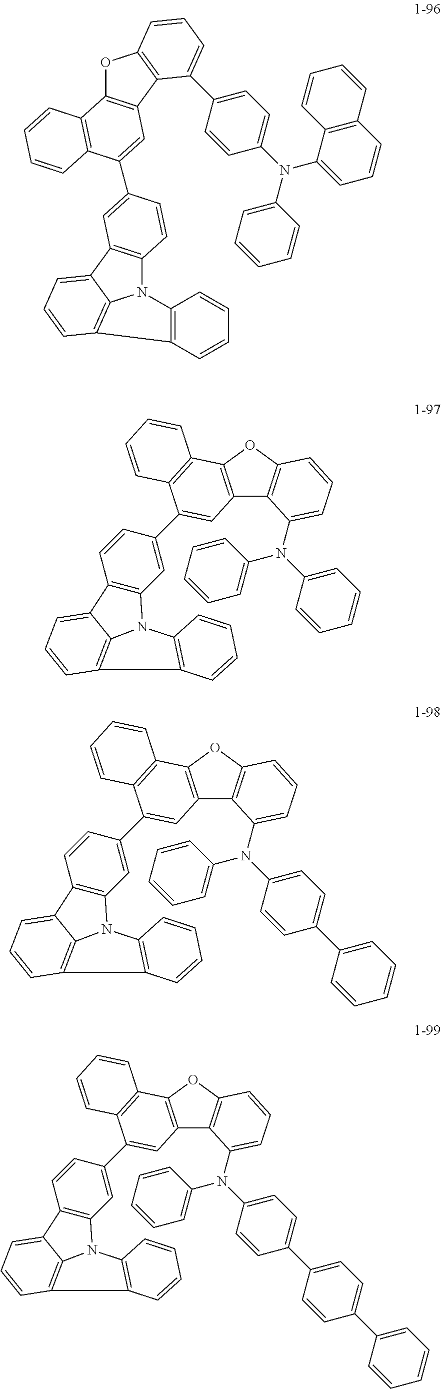 [SMILES: c1ccc(N(c2ccc(-c3cccc4oc5c6ccccc6c(-c6ccc7c(c6)c6cccc8c9ccccc9n7c86)cc5c34)cc2)c2cccc3ccccc23)cc1 ; c1ccc(N(c2ccccc2)c2cccc3oc4c5ccccc5c(-c5ccc6c7cccc8c9ccccc9n(c6c5)c87)cc4c23)cc1 ; c1ccc(-c2ccc(N(c3ccccc3)c3cccc4oc5c6ccccc6c(-c6ccc7c8cccc9c%10ccccc%10n(c7c6)c98)cc5c34)cc2)cc1 ; c1ccc(-c2ccc(-c3ccc(N(c4ccccc4)c4cccc5oc6c7ccccc7c(-c7ccc8c9cccc%10c%11ccccc%11n(c8c7)c%109)cc6c45)cc3)cc2)cc1]

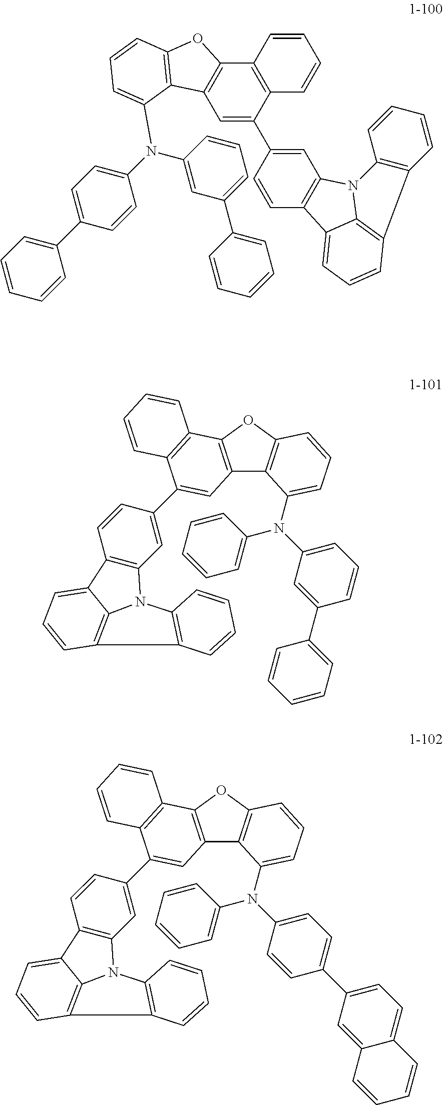 [SMILES: c1ccc(-c2ccc(N(c3cccc(-c4ccccc4)c3)c3cccc4oc5c6ccccc6c(-c6ccc7c8cccc9c%10ccccc%10n(c7c6)c98)cc5c34)cc2)cc1 ; c1ccc(-c2cccc(N(c3ccccc3)c3cccc4oc5c6ccccc6c(-c6ccc7c8cccc9c%10ccccc%10n(c7c6)c98)cc5c34)c2)cc1 ; c1ccc(N(c2ccc(-c3ccc4ccccc4c3)cc2)c2cccc3oc4c5ccccc5c(-c5ccc6c7cccc8c9ccccc9n(c6c5)c87)cc4c23)cc1]

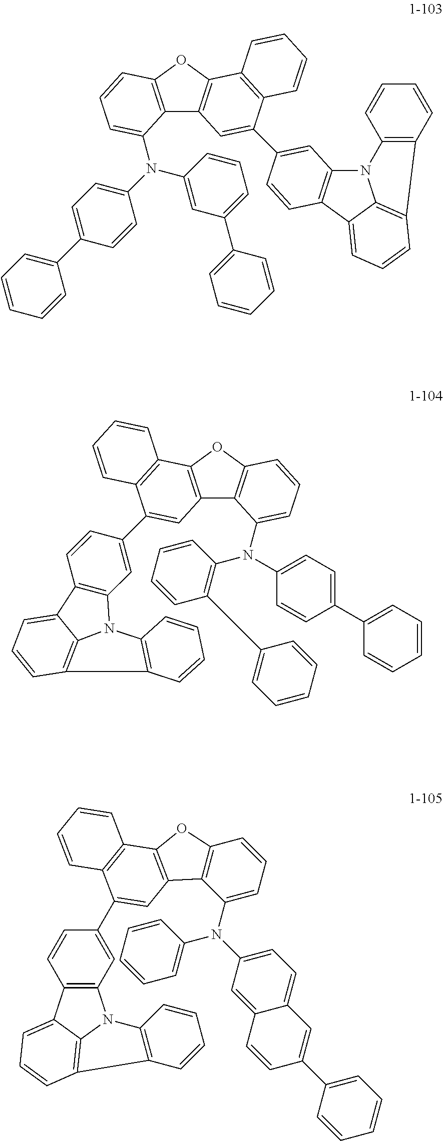 [SMILES: c1ccc(-c2ccc(N(c3cccc(-c4ccccc4)c3)c3cccc4oc5c6ccccc6c(-c6ccc7c8cccc9c%10ccccc%10n(c7c6)c98)cc5c34)cc2)cc1 ; c1ccc(-c2ccc(N(c3ccccc3-c3ccccc3)c3cccc4oc5c6ccccc6c(-c6ccc7c8cccc9c%10ccccc%10n(c7c6)c98)cc5c34)cc2)cc1 ; c1ccc(-c2ccc3cc(N(c4ccccc4)c4cccc5oc6c7ccccc7c(-c7ccc8c9cccc%10c%11ccccc%11n(c8c7)c%109)cc6c45)ccc3c2)cc1]

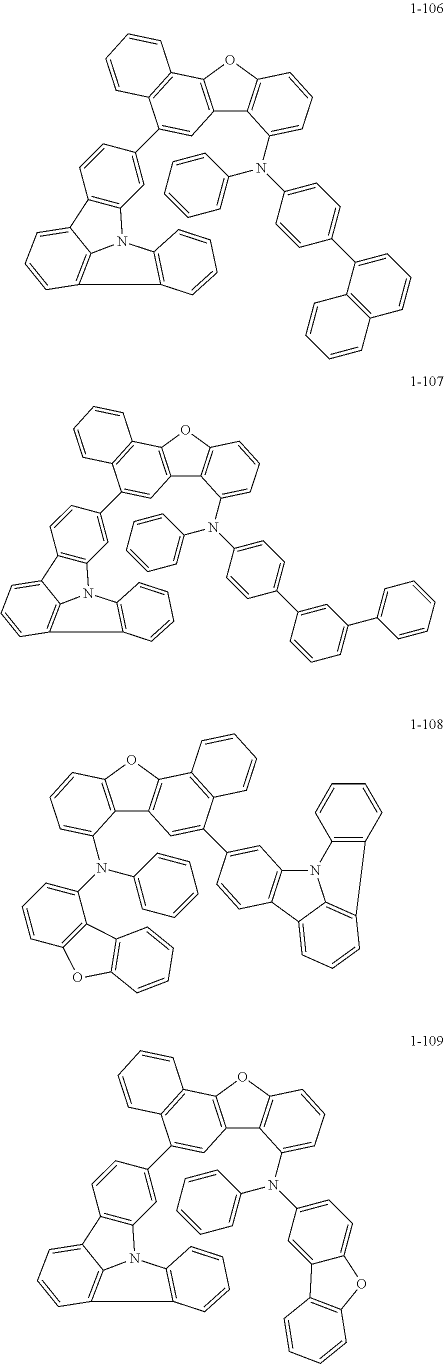 [SMILES: c1ccc(N(c2ccc(-c3cccc4ccccc34)cc2)c2cccc3oc4c5ccccc5c(-c5ccc6c7cccc8c9ccccc9n(c6c5)c87)cc4c23)cc1 ; c1ccc(-c2cccc(-c3ccc(N(c4ccccc4)c4cccc5oc6c7ccccc7c(-c7ccc8c9cccc%10c%11ccccc%11n(c8c7)c%109)cc6c45)cc3)c2)cc1 ; c1ccc(N(c2cccc3oc4ccccc4c23)c2cccc3oc4c5ccccc5c(-c5ccc6c7cccc8c9ccccc9n(c6c5)c87)cc4c23)cc1 ; c1ccc(N(c2ccc3oc4ccccc4c3c2)c2cccc3oc4c5ccccc5c(-c5ccc6c7cccc8c9ccccc9n(c6c5)c87)cc4c23)cc1]

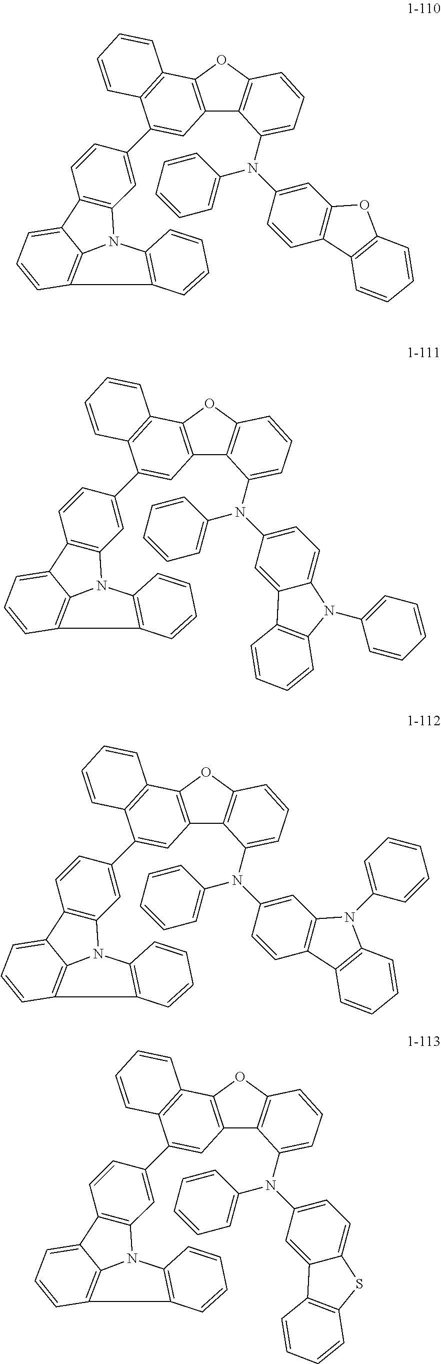 [SMILES: c1ccc(N(c2ccc3c(c2)oc2ccccc23)c2cccc3oc4c5ccccc5c(-c5ccc6c7cccc8c9ccccc9n(c6c5)c87)cc4c23)cc1 ; c1ccc(N(c2ccc3c(c2)c2ccccc2n3-c2ccccc2)c2cccc3oc4c5ccccc5c(-c5ccc6c7cccc8c9ccccc9n(c6c5)c87)cc4c23)cc1 ; c1ccc(N(c2ccc3c4ccccc4n(-c4ccccc4)c3c2)c2cccc3oc4c5ccccc5c(-c5ccc6c7cccc8c9ccccc9n(c6c5)c87)cc4c23)cc1 ; c1ccc(N(c2ccc3sc4ccccc4c3c2)c2cccc3oc4c5ccccc5c(-c5ccc6c7cccc8c9ccccc9n(c6c5)c87)cc4c23)cc1]

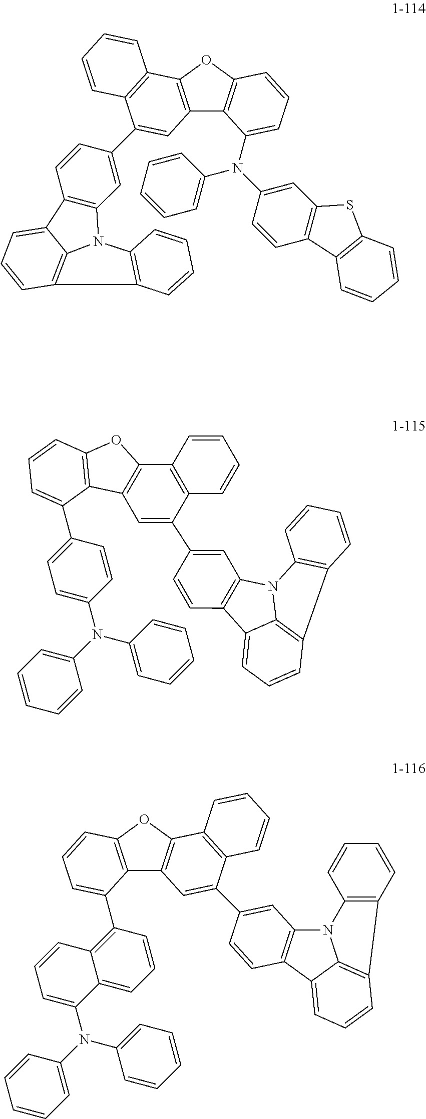 [SMILES: c1ccc(N(c2ccc3c(c2)sc2ccccc23)c2cccc3oc4c5ccccc5c(-c5ccc6c7cccc8c9ccccc9n(c6c5)c87)cc4c23)cc1 ; c1ccc(N(c2ccccc2)c2ccc(-c3cccc4oc5c6ccccc6c(-c6ccc7c8cccc9c%10ccccc%10n(c7c6)c98)cc5c34)cc2)cc1 ; c1ccc(N(c2ccccc2)c2cccc3c(-c4cccc5oc6c7ccccc7c(-c7ccc8c9cccc%10c%11ccccc%11n(c8c7)c%109)cc6c45)cccc23)cc1]

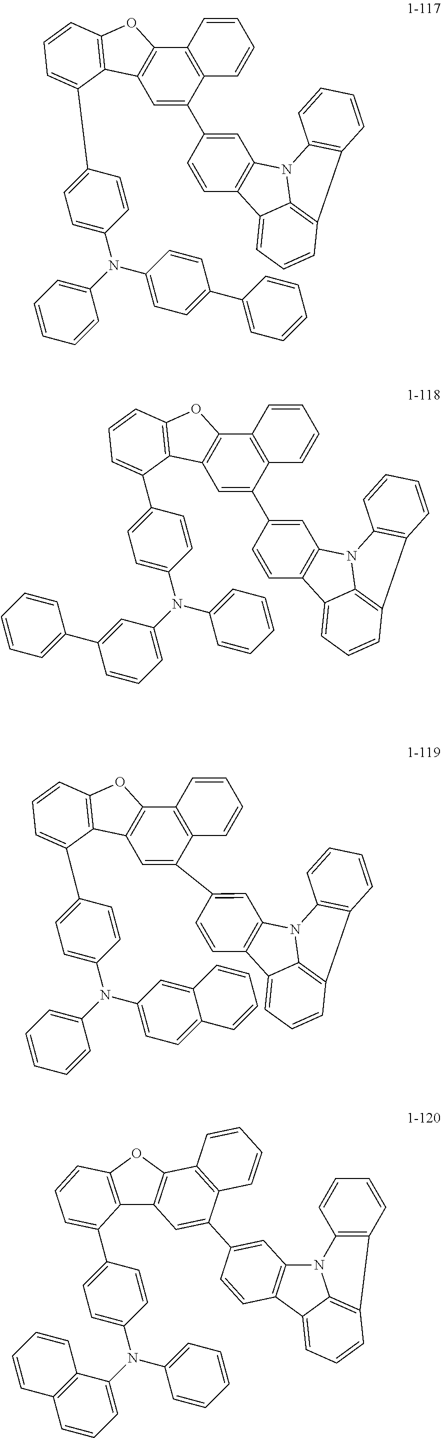 [SMILES: c1ccc(-c2ccc(N(c3ccccc3)c3ccc(-c4cccc5oc6c7ccccc7c(-c7ccc8c9cccc%10c%11ccccc%11n(c8c7)c%109)cc6c45)cc3)cc2)cc1 ; c1ccc(-c2cccc(N(c3ccccc3)c3ccc(-c4cccc5oc6c7ccccc7c(-c7ccc8c9cccc%10c%11ccccc%11n(c8c7)c%109)cc6c45)cc3)c2)cc1 ; c1ccc(N(c2ccc(-c3cccc4oc5c6ccccc6c(-c6ccc7c8cccc9c%10ccccc%10n(c7c6)c98)cc5c34)cc2)c2ccc3ccccc3c2)cc1 ; c1ccc(N(c2ccc(-c3cccc4oc5c6ccccc6c(-c6ccc7c8cccc9c%10ccccc%10n(c7c6)c98)cc5c34)cc2)c2cccc3ccccc23)cc1]

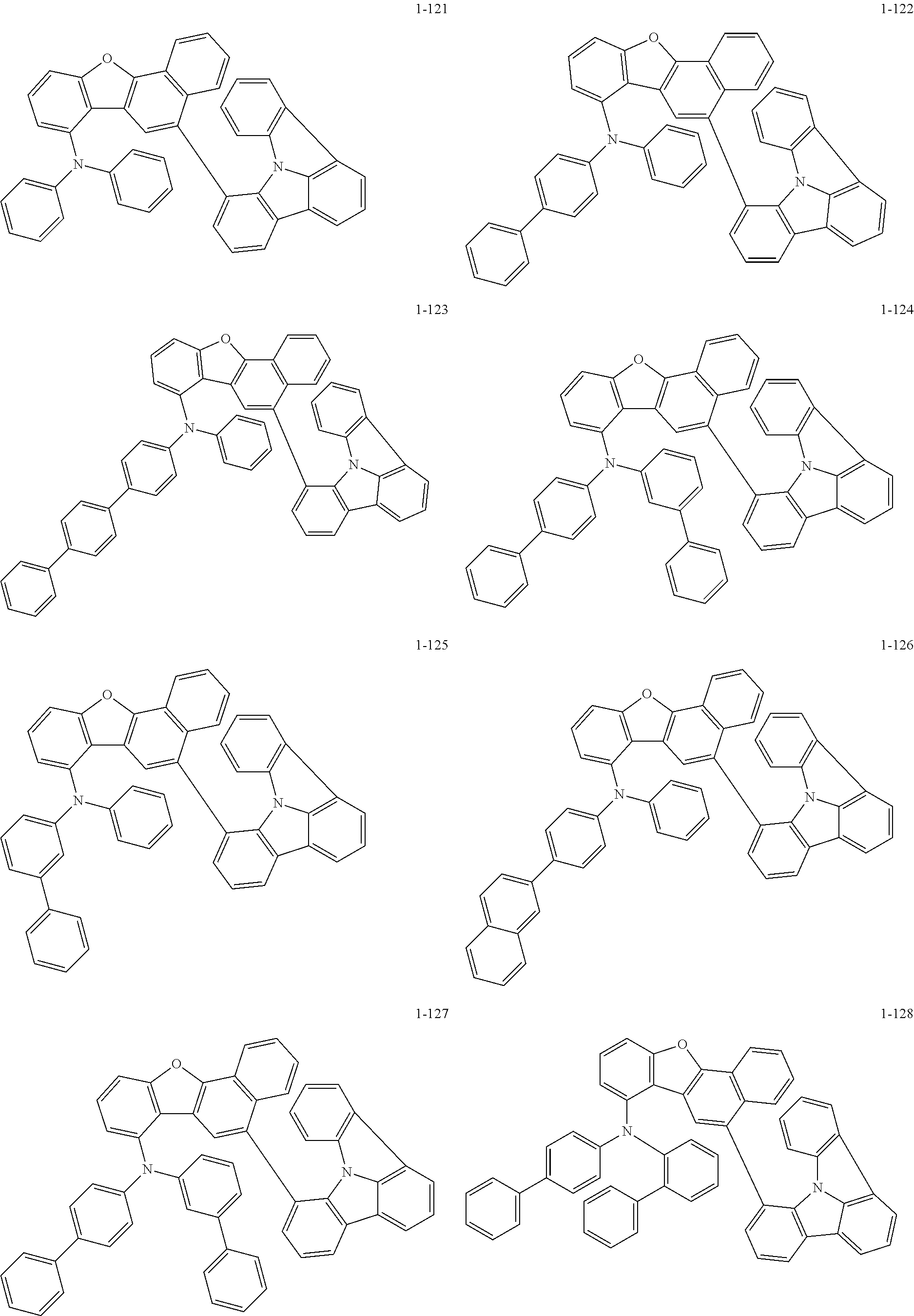

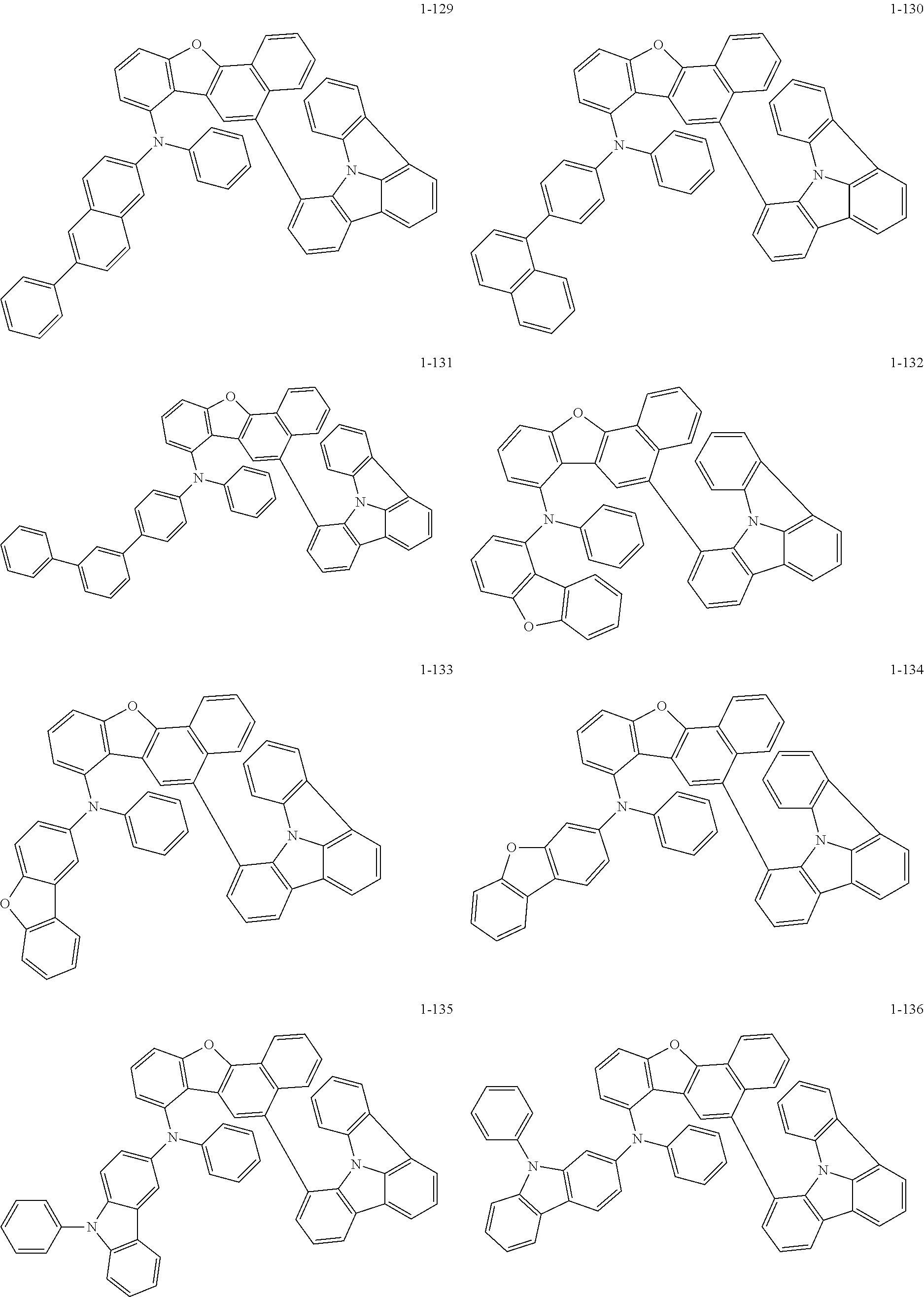

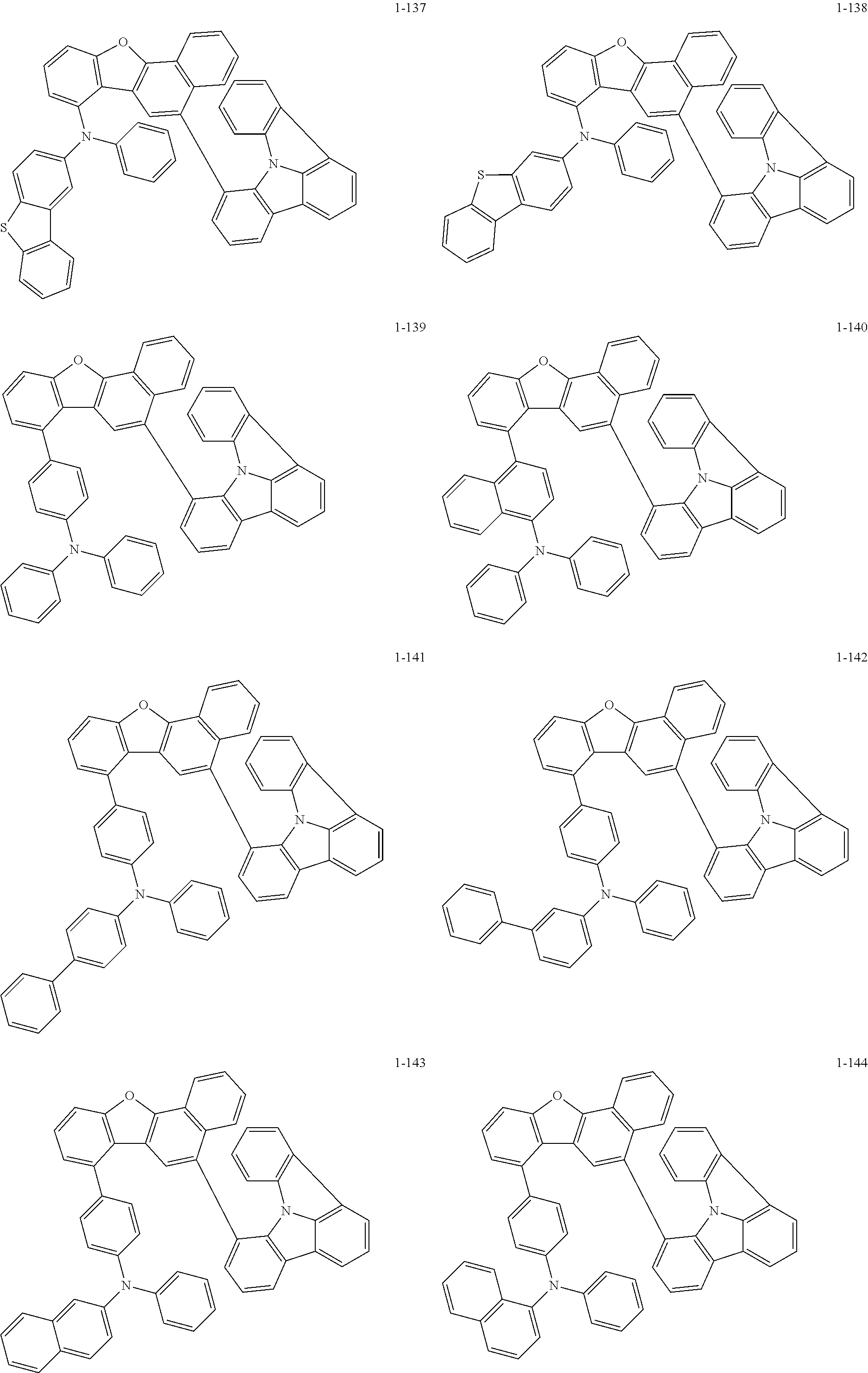

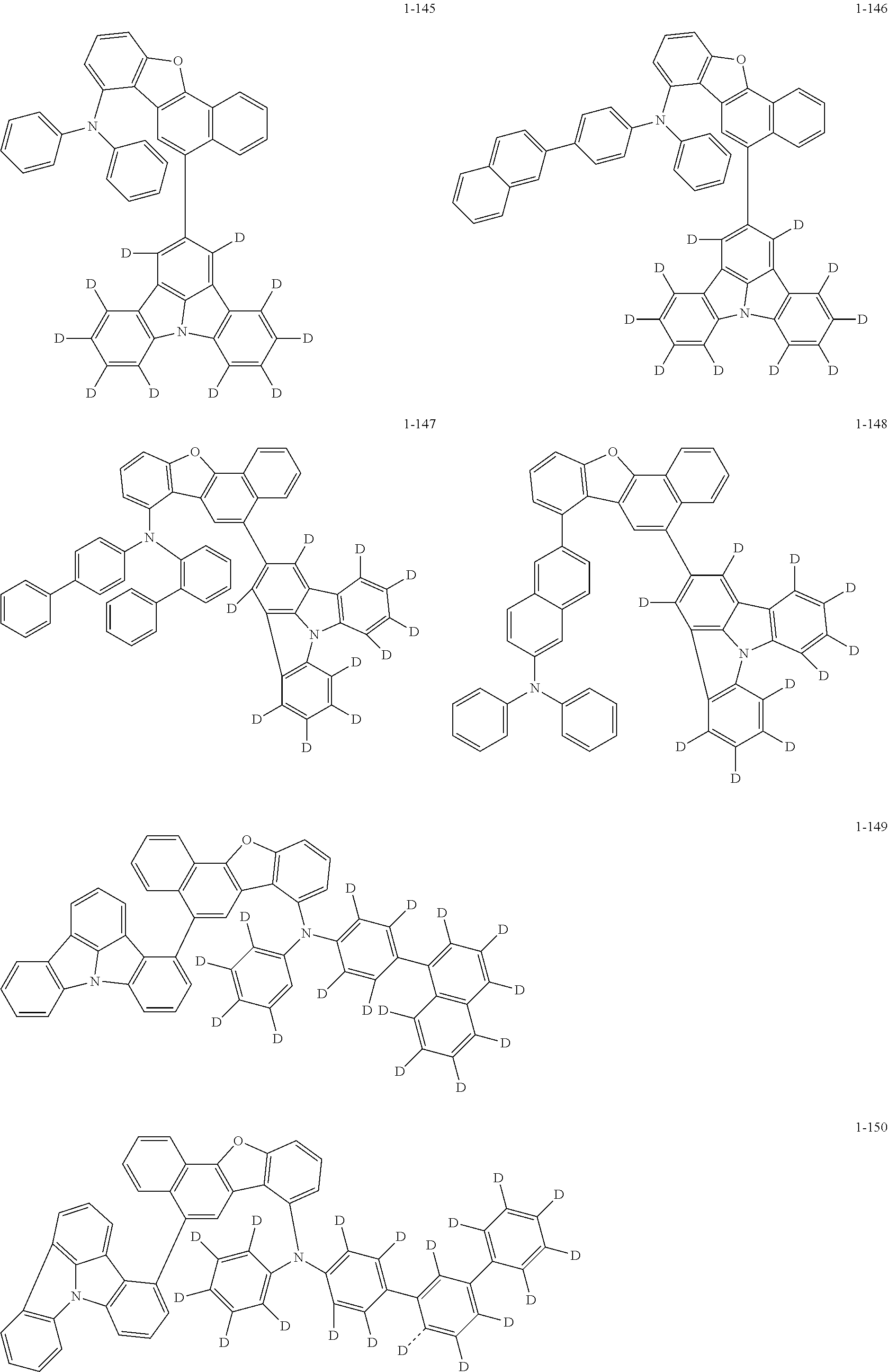

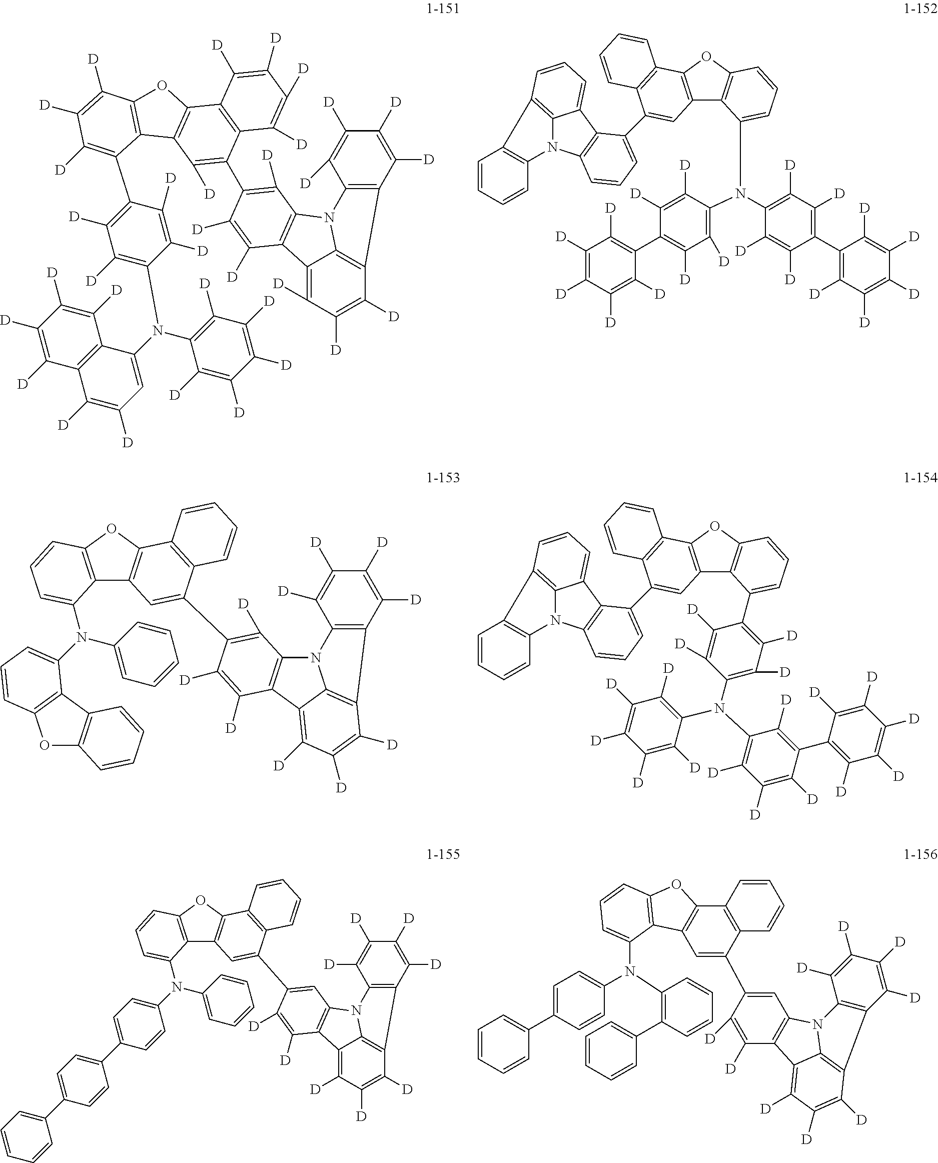

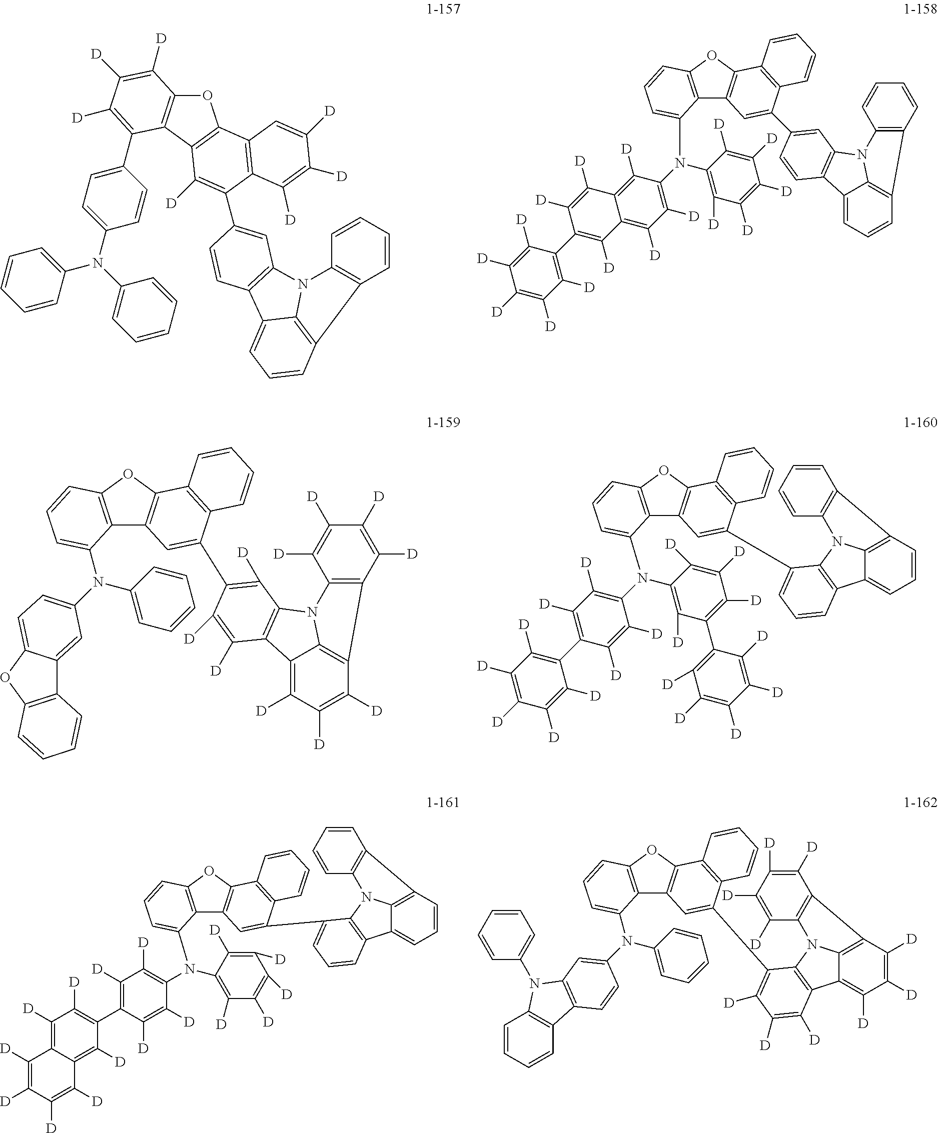

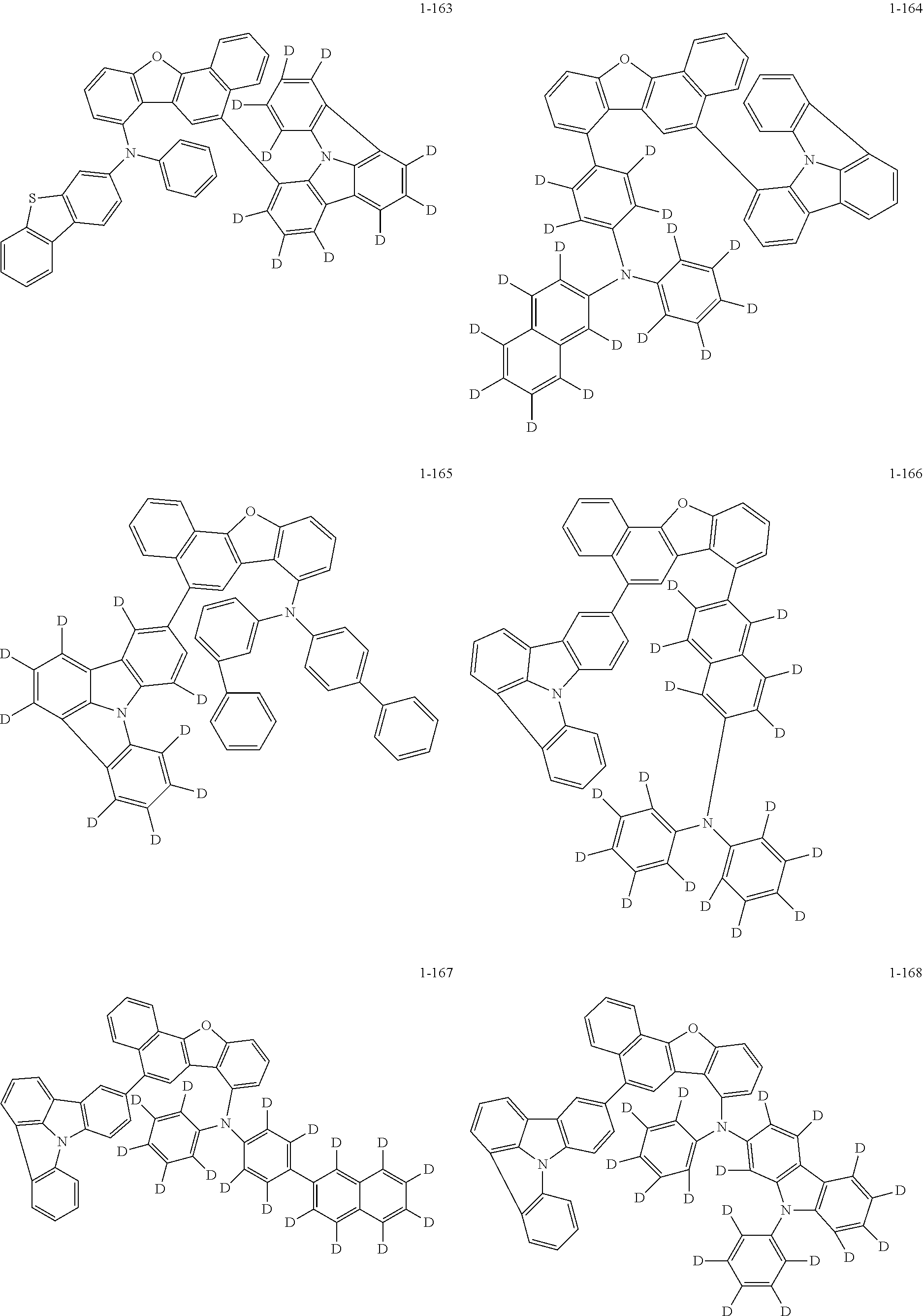

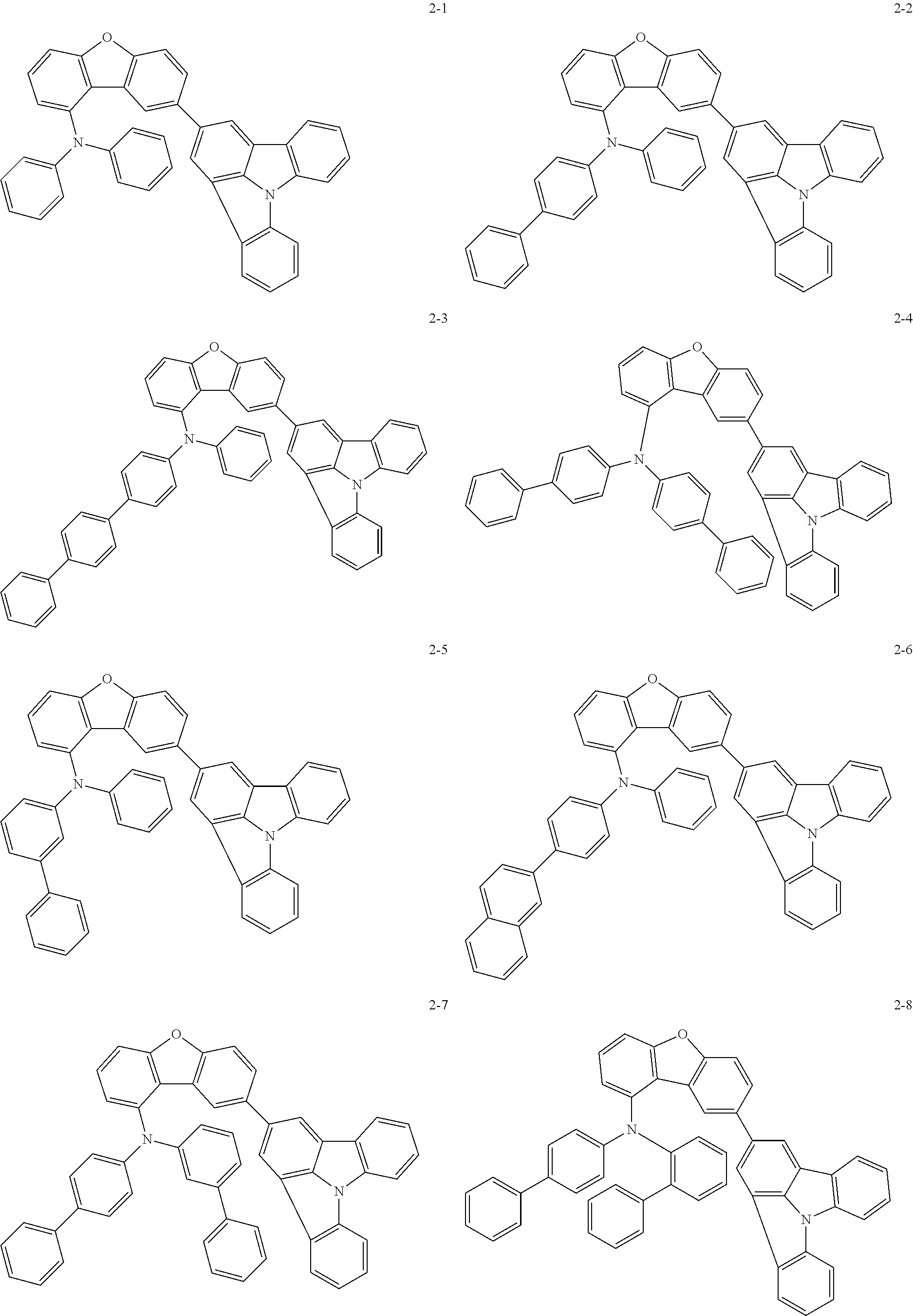

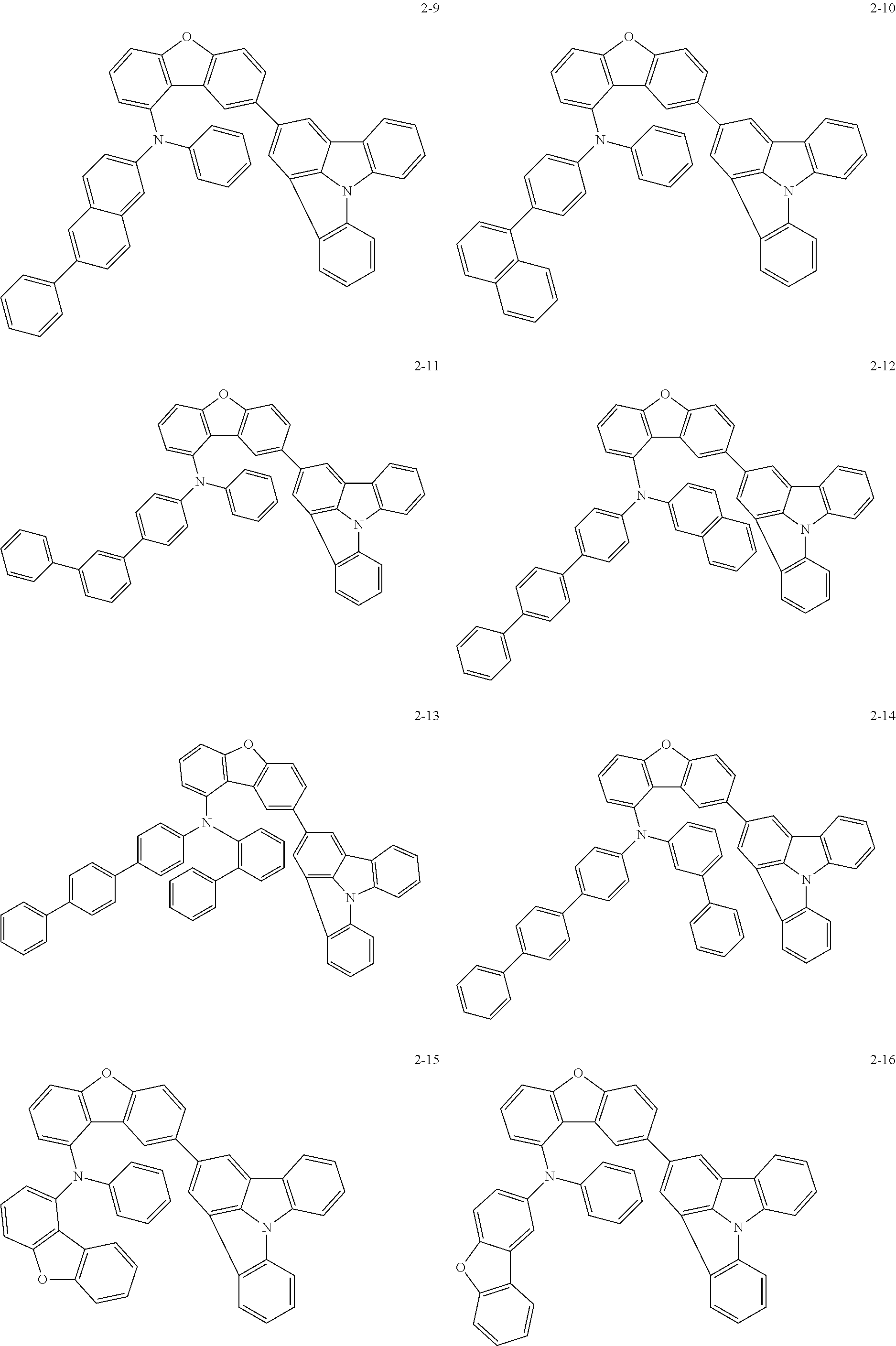

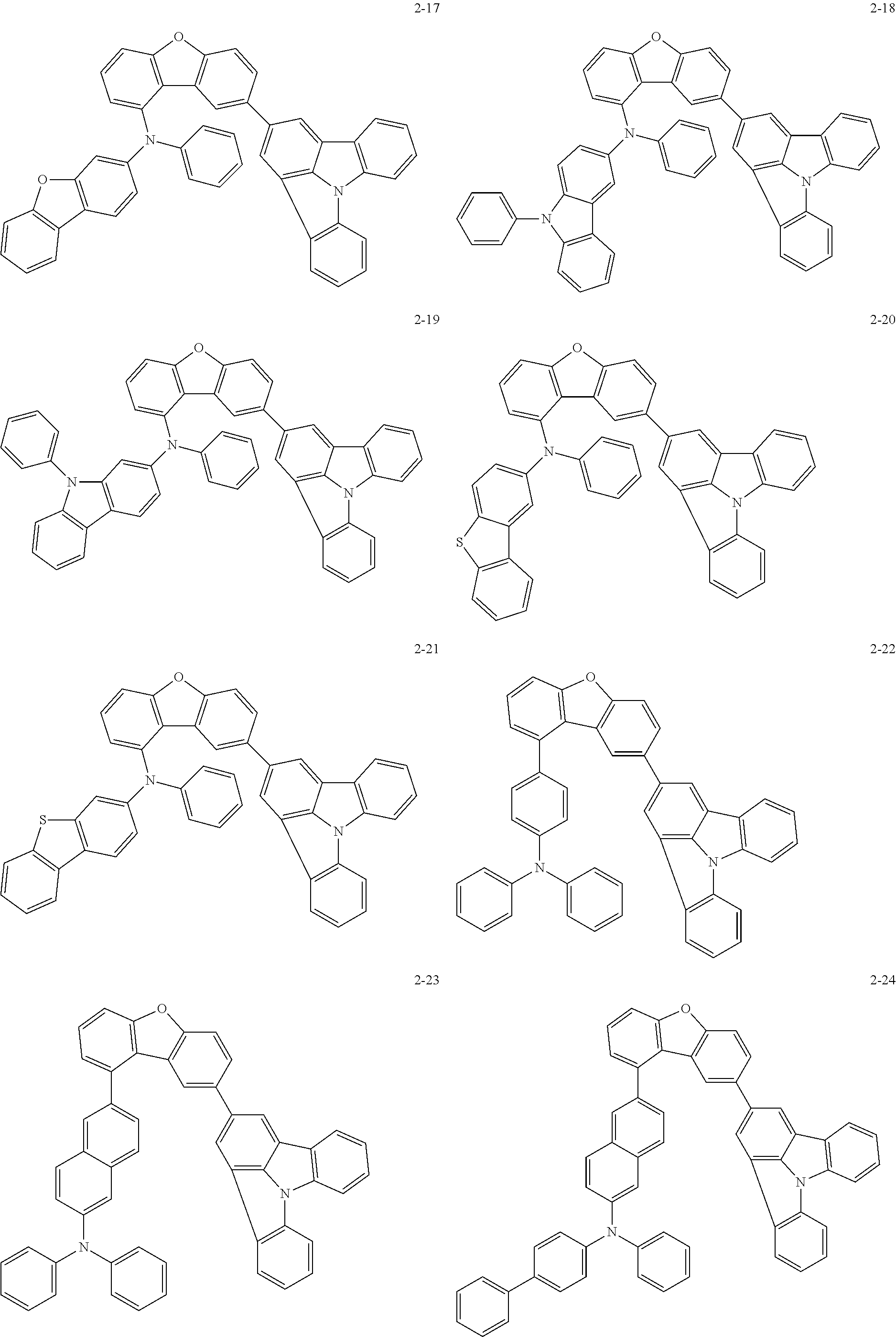

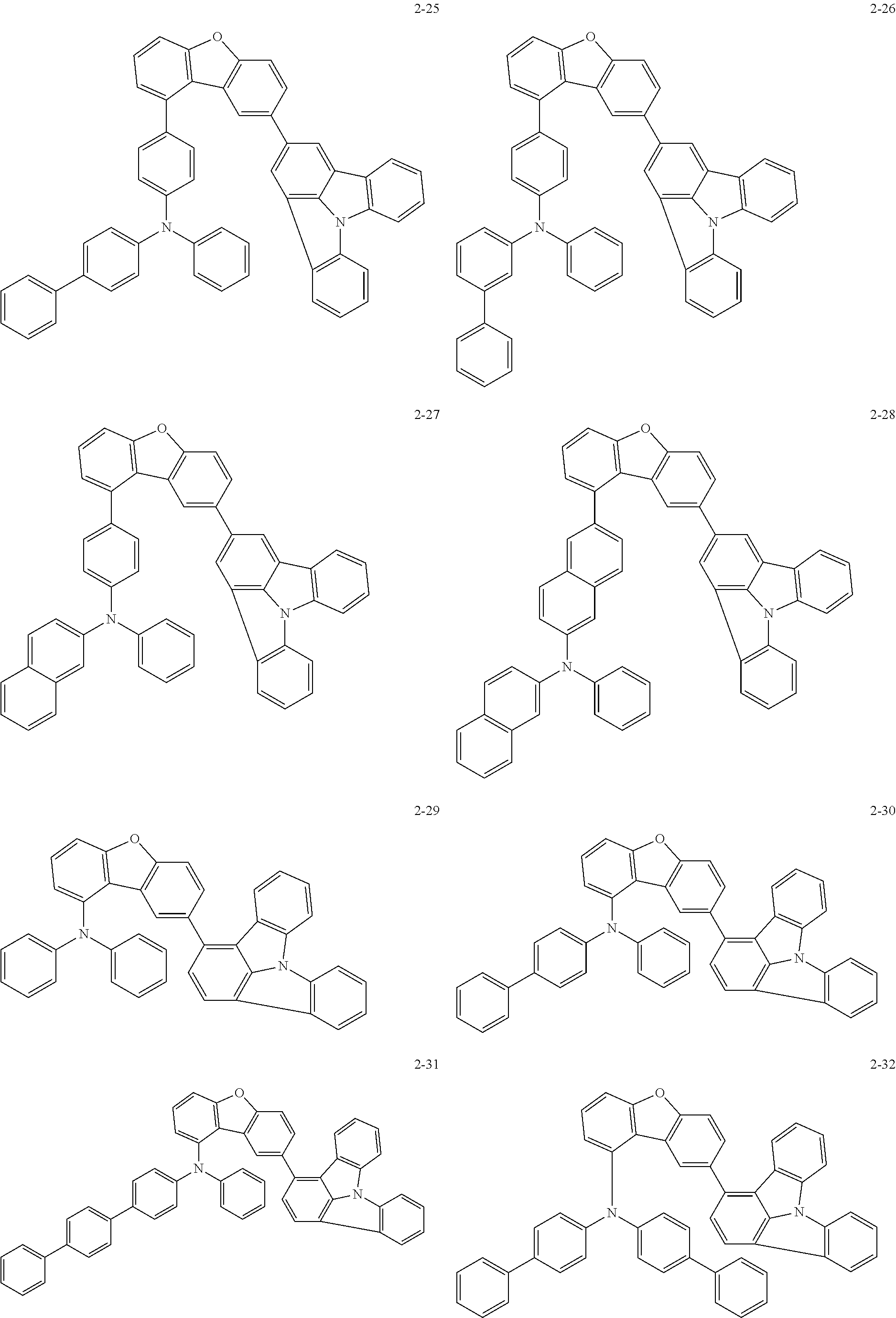

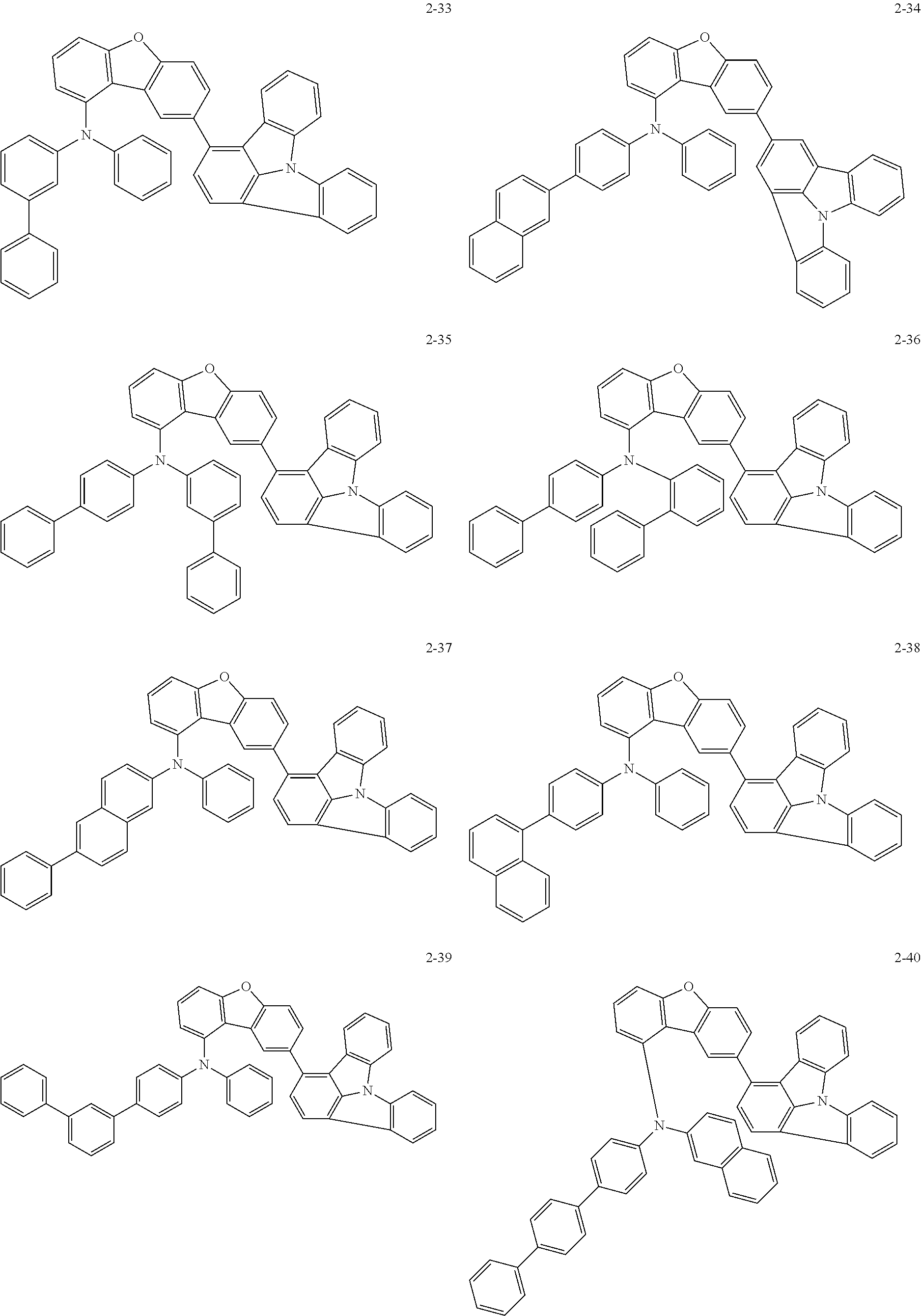

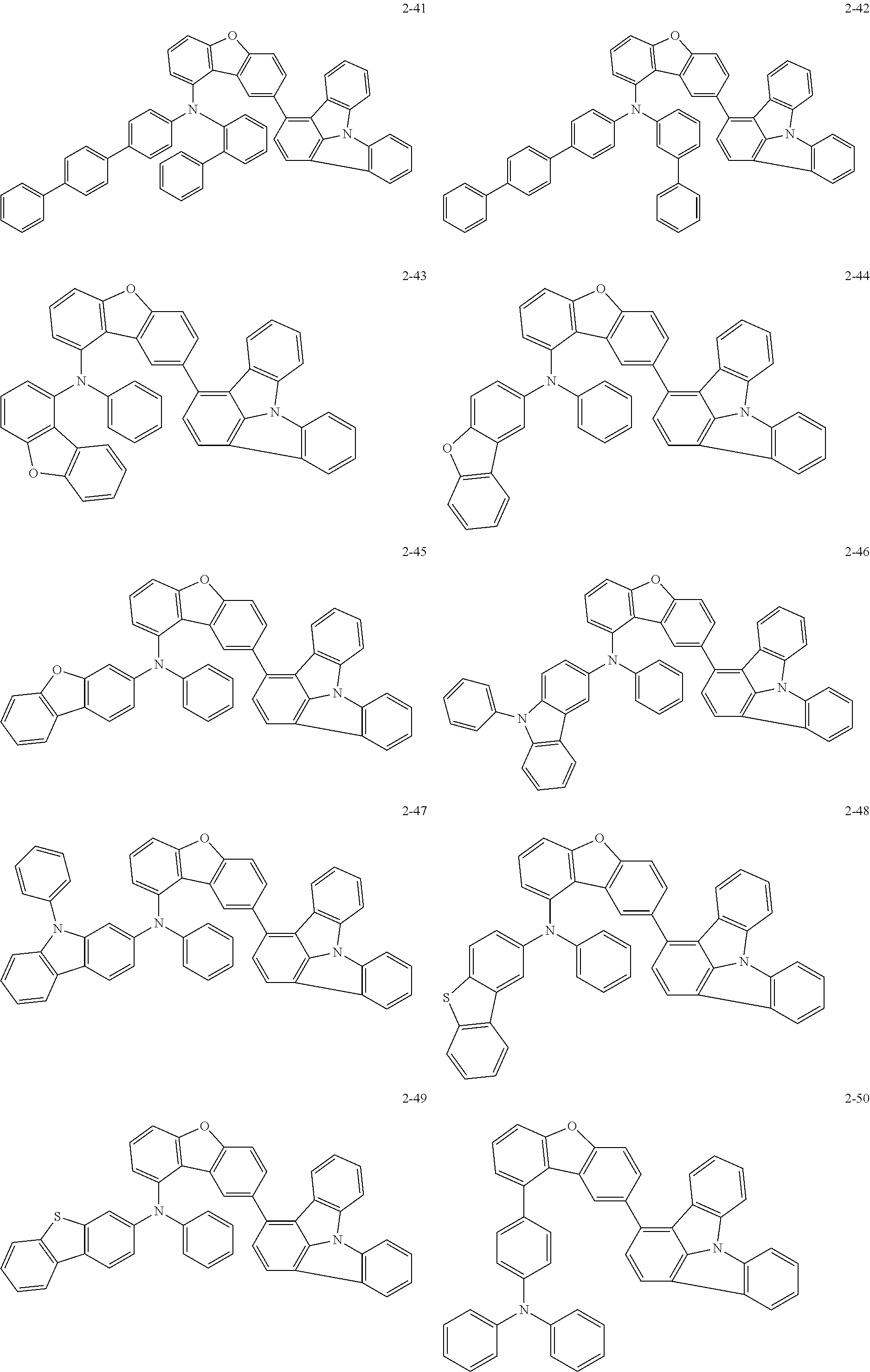

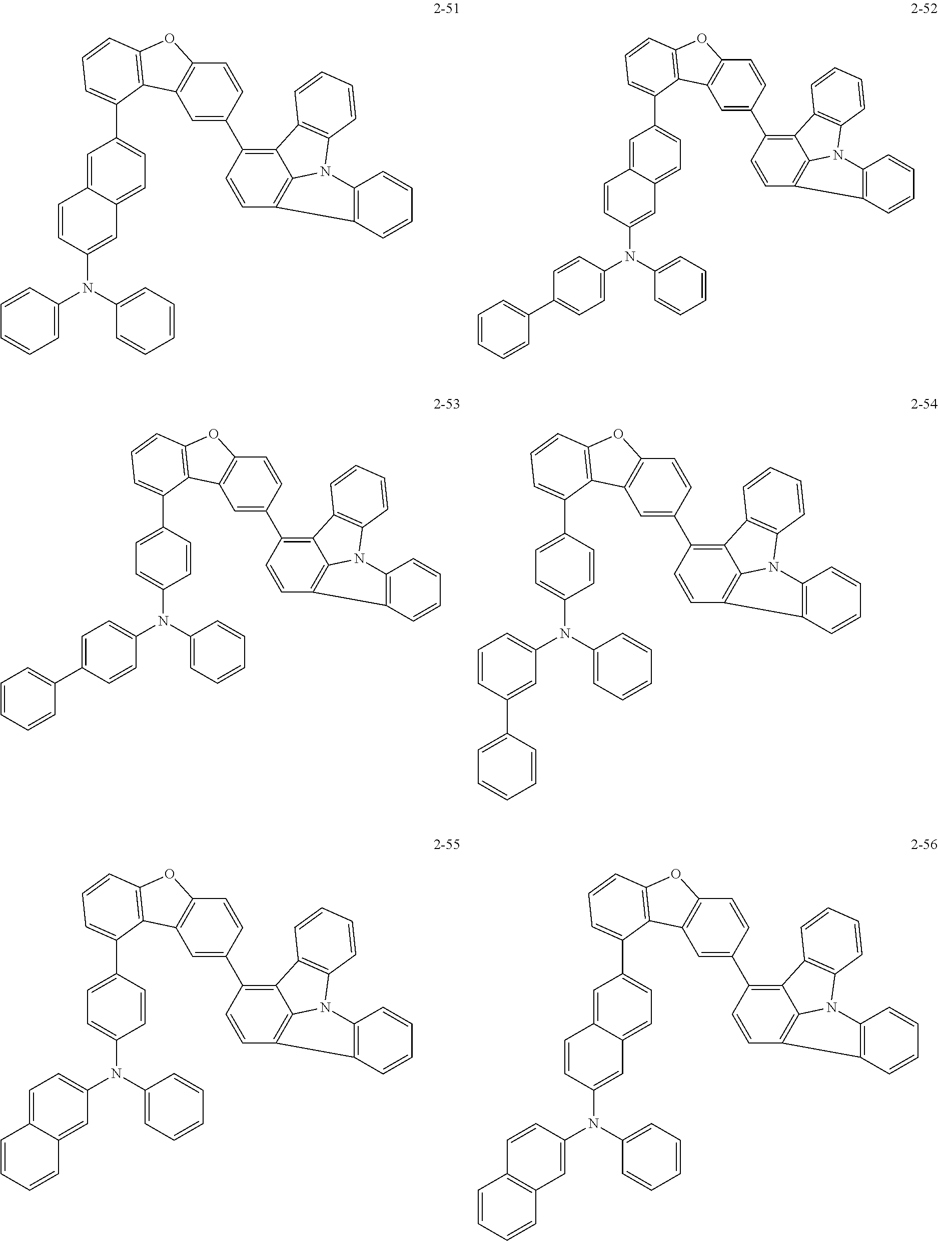

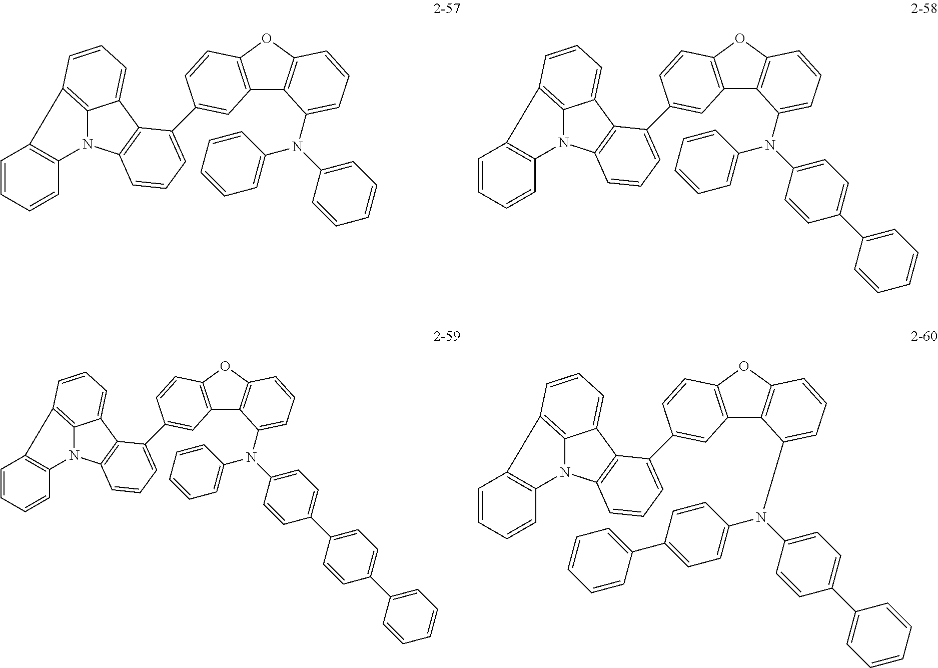 [SMILES: c1ccc(N(c2ccccc2)c2cccc3oc4ccc(-c5cccc6c5c5cccc7c8ccccc8n6c75)cc4c23)cc1 ; c1ccc(-c2ccc(N(c3ccccc3)c3cccc4oc5ccc(-c6cccc7c6c6cccc8c9ccccc9n7c86)cc5c34)cc2)cc1 ; c1ccc(-c2ccc(-c3ccc(N(c4ccccc4)c4cccc5oc6ccc(-c7cccc8c7c7cccc9c%10ccccc%10n8c97)cc6c45)cc3)cc2)cc1 ; c1ccc(-c2ccc(N(c3ccc(-c4ccccc4)cc3)c3cccc4oc5ccc(-c6cccc7c6c6cccc8c9ccccc9n7c86)cc5c34)cc2)cc1]

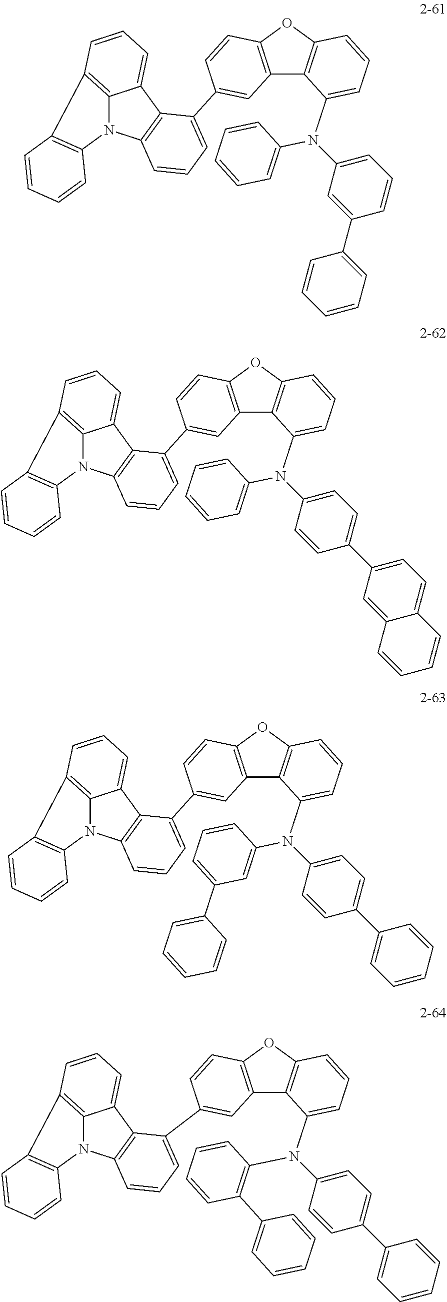 [SMILES: c1ccc(-c2cccc(N(c3ccccc3)c3cccc4oc5ccc(-c6cccc7c6c6cccc8c9ccccc9n7c86)cc5c34)c2)cc1 ; c1ccc(N(c2ccc(-c3ccc4ccccc4c3)cc2)c2cccc3oc4ccc(-c5cccc6c5c5cccc7c8ccccc8n6c75)cc4c23)cc1 ; c1ccc(-c2ccc(N(c3cccc(-c4ccccc4)c3)c3cccc4oc5ccc(-c6cccc7c6c6cccc8c9ccccc9n7c86)cc5c34)cc2)cc1 ; c1ccc(-c2ccc(N(c3ccccc3-c3ccccc3)c3cccc4oc5ccc(-c6cccc7c6c6cccc8c9ccccc9n7c86)cc5c34)cc2)cc1]

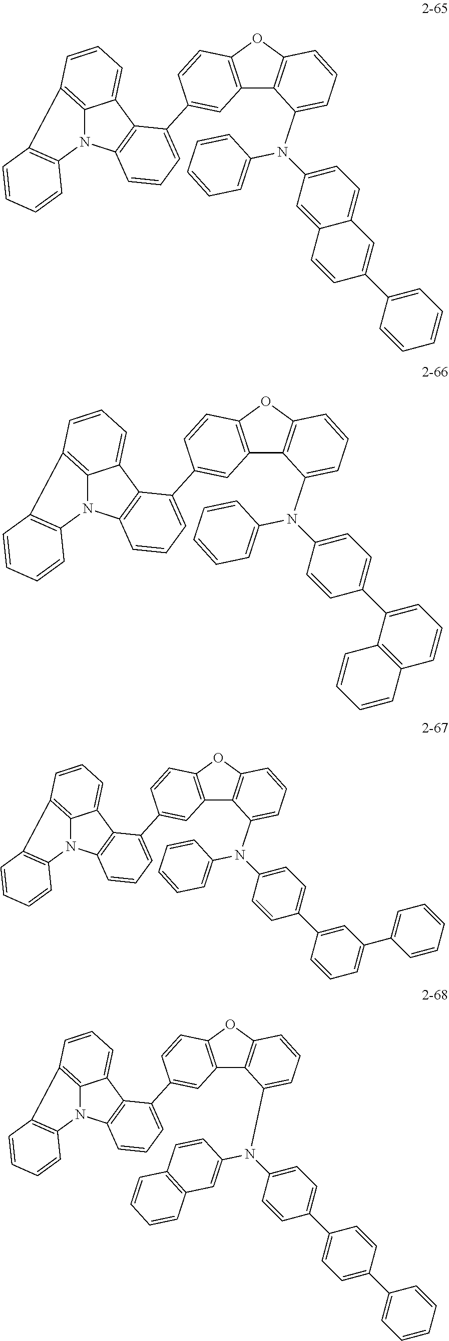 [SMILES: c1ccc(-c2ccc3cc(N(c4ccccc4)c4cccc5oc6ccc(-c7cccc8c7c7cccc9c%10ccccc%10n8c97)cc6c45)ccc3c2)cc1 ; c1ccc(N(c2ccc(-c3cccc4ccccc34)cc2)c2cccc3oc4ccc(-c5cccc6c5c5cccc7c8ccccc8n6c75)cc4c23)cc1 ; c1ccc(-c2cccc(-c3ccc(N(c4ccccc4)c4cccc5oc6ccc(-c7cccc8c7c7cccc9c%10ccccc%10n8c97)cc6c45)cc3)c2)cc1 ; c1ccc(-c2ccc(-c3ccc(N(c4ccc5ccccc5c4)c4cccc5oc6ccc(-c7cccc8c7c7cccc9c%10ccccc%10n8c97)cc6c45)cc3)cc2)cc1]

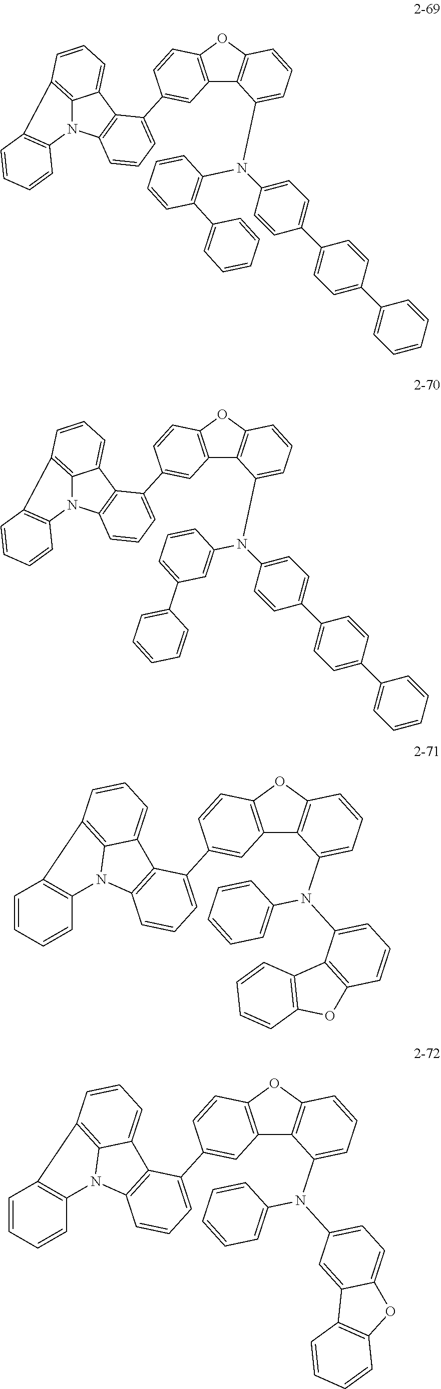 [SMILES: c1ccc(-c2ccc(-c3ccc(N(c4ccccc4-c4ccccc4)c4cccc5oc6ccc(-c7cccc8c7c7cccc9c%10ccccc%10n8c97)cc6c45)cc3)cc2)cc1 ; c1ccc(-c2ccc(-c3ccc(N(c4cccc(-c5ccccc5)c4)c4cccc5oc6ccc(-c7cccc8c7c7cccc9c%10ccccc%10n8c97)cc6c45)cc3)cc2)cc1 ; c1ccc(N(c2cccc3oc4ccccc4c23)c2cccc3oc4ccc(-c5cccc6c5c5cccc7c8ccccc8n6c75)cc4c23)cc1 ; c1ccc(N(c2ccc3oc4ccccc4c3c2)c2cccc3oc4ccc(-c5cccc6c5c5cccc7c8ccccc8n6c75)cc4c23)cc1]

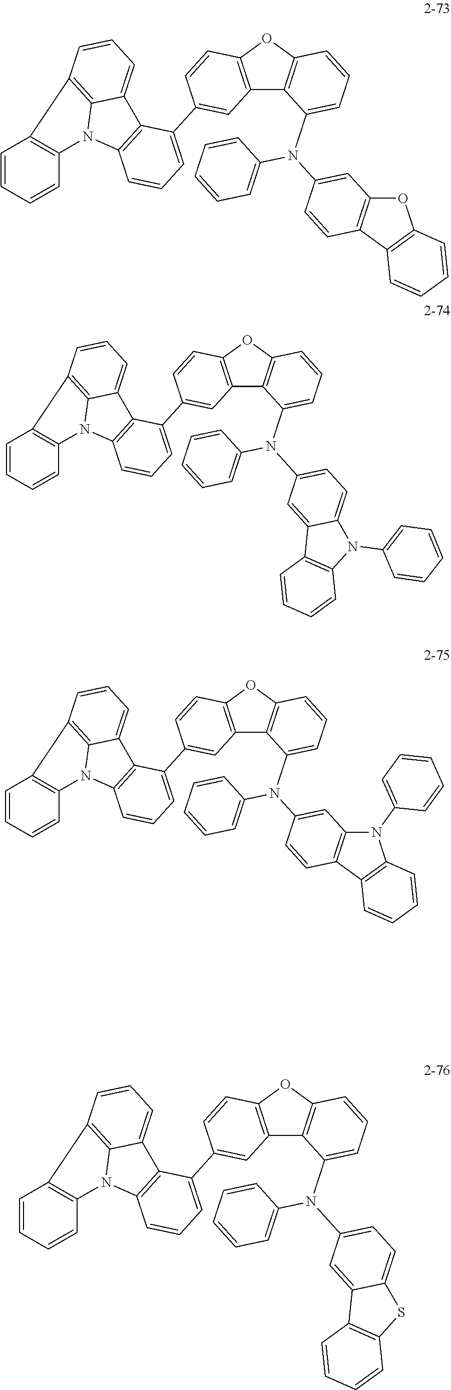 [SMILES: c1ccc(N(c2ccc3c(c2)oc2ccccc23)c2cccc3oc4ccc(-c5cccc6c5c5cccc7c8ccccc8n6c75)cc4c23)cc1 ; c1ccc(N(c2ccc3c(c2)c2ccccc2n3-c2ccccc2)c2cccc3oc4ccc(-c5cccc6c5c5cccc7c8ccccc8n6c75)cc4c23)cc1 ; c1ccc(N(c2ccc3c4ccccc4n(-c4ccccc4)c3c2)c2cccc3oc4ccc(-c5cccc6c5c5cccc7c8ccccc8n6c75)cc4c23)cc1 ; c1ccc(N(c2ccc3sc4ccccc4c3c2)c2cccc3oc4ccc(-c5cccc6c5c5cccc7c8ccccc8n6c75)cc4c23)cc1]

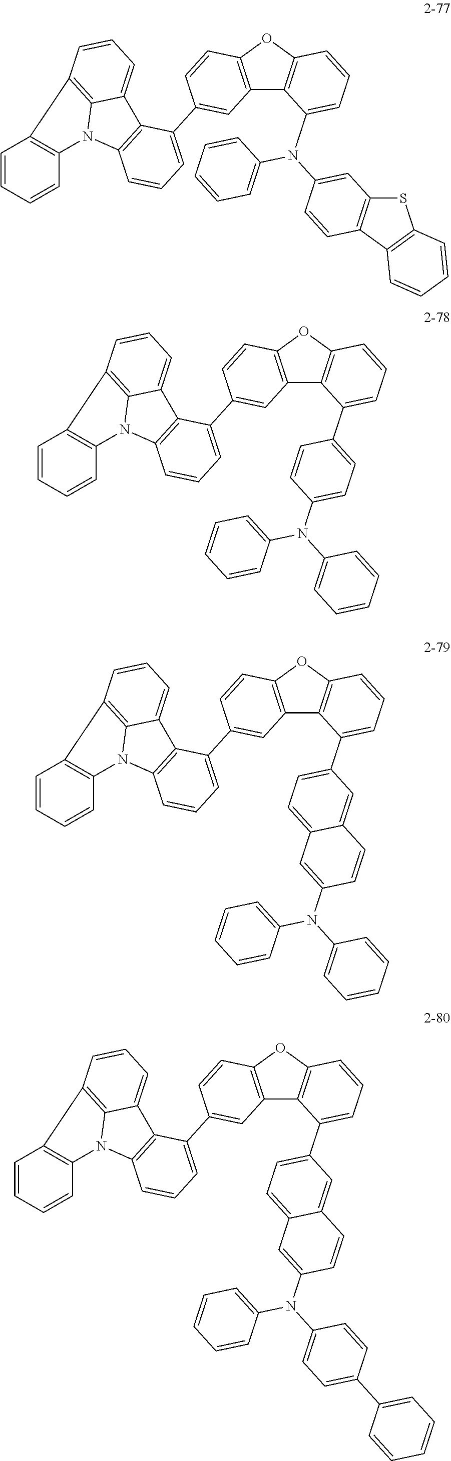 [SMILES: c1ccc(N(c2ccc3c(c2)sc2ccccc23)c2cccc3oc4ccc(-c5cccc6c5c5cccc7c8ccccc8n6c75)cc4c23)cc1 ; c1ccc(N(c2ccccc2)c2ccc(-c3cccc4oc5ccc(-c6cccc7c6c6cccc8c9ccccc9n7c86)cc5c34)cc2)cc1 ; c1ccc(N(c2ccccc2)c2ccc3cc(-c4cccc5oc6ccc(-c7cccc8c7c7cccc9c%10ccccc%10n8c97)cc6c45)ccc3c2)cc1 ; c1ccc(-c2ccc(N(c3ccccc3)c3ccc4cc(-c5cccc6oc7ccc(-c8cccc9c8c8cccc%10c%11ccccc%11n9c%108)cc7c56)ccc4c3)cc2)cc1]

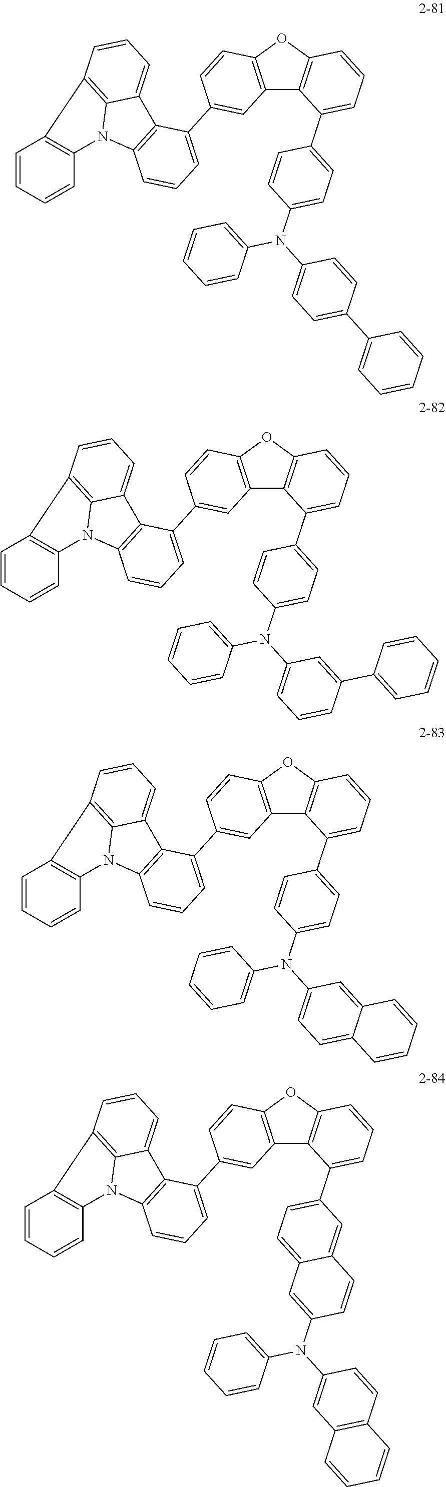

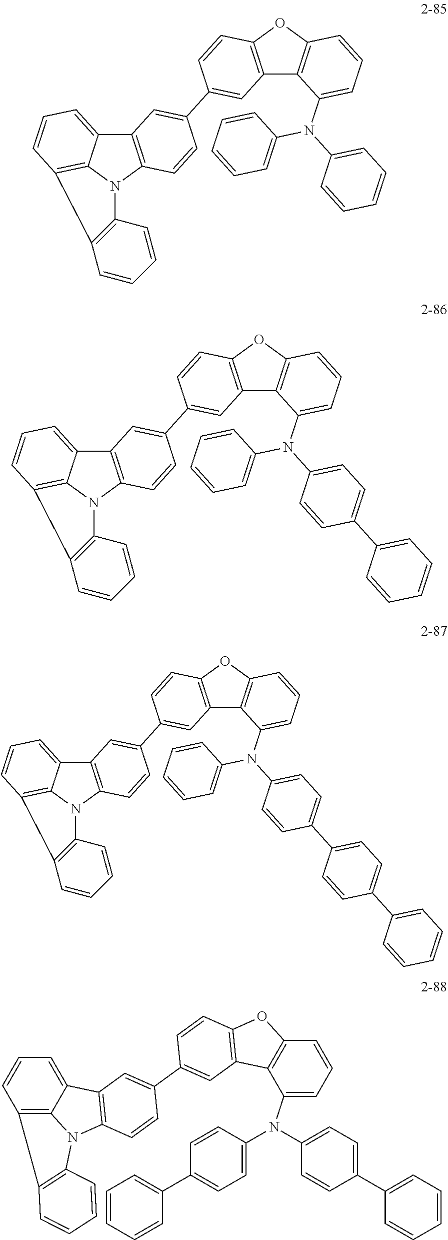 [SMILES: c1ccc(N(c2ccccc2)c2cccc3oc4ccc(-c5ccc6c(c5)c5cccc7c8ccccc8n6c75)cc4c23)cc1 ; c1ccc(-c2ccc(N(c3ccccc3)c3cccc4oc5ccc(-c6ccc7c(c6)c6cccc8c9ccccc9n7c86)cc5c34)cc2)cc1 ; c1ccc(-c2ccc(-c3ccc(N(c4ccccc4)c4cccc5oc6ccc(-c7ccc8c(c7)c7cccc9c%10ccccc%10n8c97)cc6c45)cc3)cc2)cc1 ; c1ccc(-c2ccc(N(c3ccc(-c4ccccc4)cc3)c3cccc4oc5ccc(-c6ccc7c(c6)c6cccc8c9ccccc9n7c86)cc5c34)cc2)cc1]

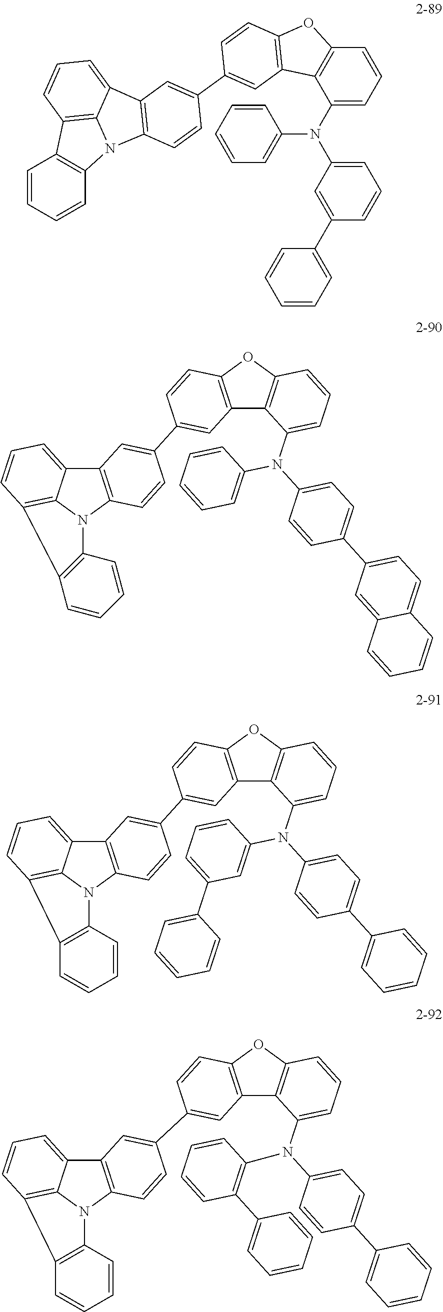 [SMILES: c1ccc(-c2cccc(N(c3ccccc3)c3cccc4oc5ccc(-c6ccc7c(c6)c6cccc8c9ccccc9n7c86)cc5c34)c2)cc1 ; c1ccc(N(c2ccc(-c3ccc4ccccc4c3)cc2)c2cccc3oc4ccc(-c5ccc6c(c5)c5cccc7c8ccccc8n6c75)cc4c23)cc1 ; c1ccc(-c2ccc(N(c3cccc(-c4ccccc4)c3)c3cccc4oc5ccc(-c6ccc7c(c6)c6cccc8c9ccccc9n7c86)cc5c34)cc2)cc1 ; c1ccc(-c2ccc(N(c3ccccc3-c3ccccc3)c3cccc4oc5ccc(-c6ccc7c(c6)c6cccc8c9ccccc9n7c86)cc5c34)cc2)cc1]

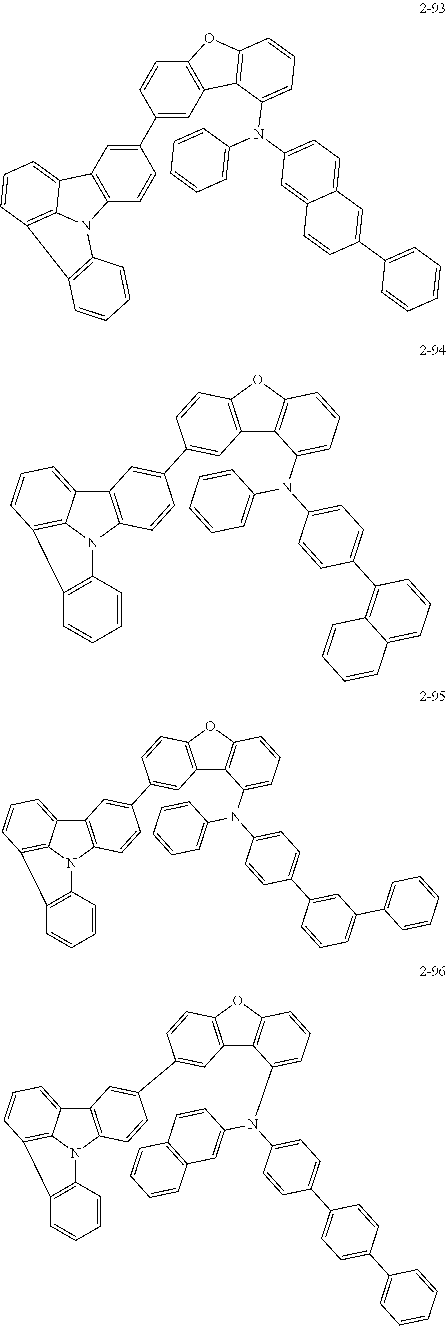 [SMILES: c1ccc(-c2ccc3cc(N(c4ccccc4)c4cccc5oc6ccc(-c7ccc8c(c7)c7cccc9c%10ccccc%10n8c97)cc6c45)ccc3c2)cc1 ; c1ccc(N(c2ccc(-c3cccc4ccccc34)cc2)c2cccc3oc4ccc(-c5ccc6c(c5)c5cccc7c8ccccc8n6c75)cc4c23)cc1 ; c1ccc(-c2cccc(-c3ccc(N(c4ccccc4)c4cccc5oc6ccc(-c7ccc8c(c7)c7cccc9c%10ccccc%10n8c97)cc6c45)cc3)c2)cc1 ; c1ccc(-c2ccc(-c3ccc(N(c4ccc5ccccc5c4)c4cccc5oc6ccc(-c7ccc8c(c7)c7cccc9c%10ccccc%10n8c97)cc6c45)cc3)cc2)cc1]

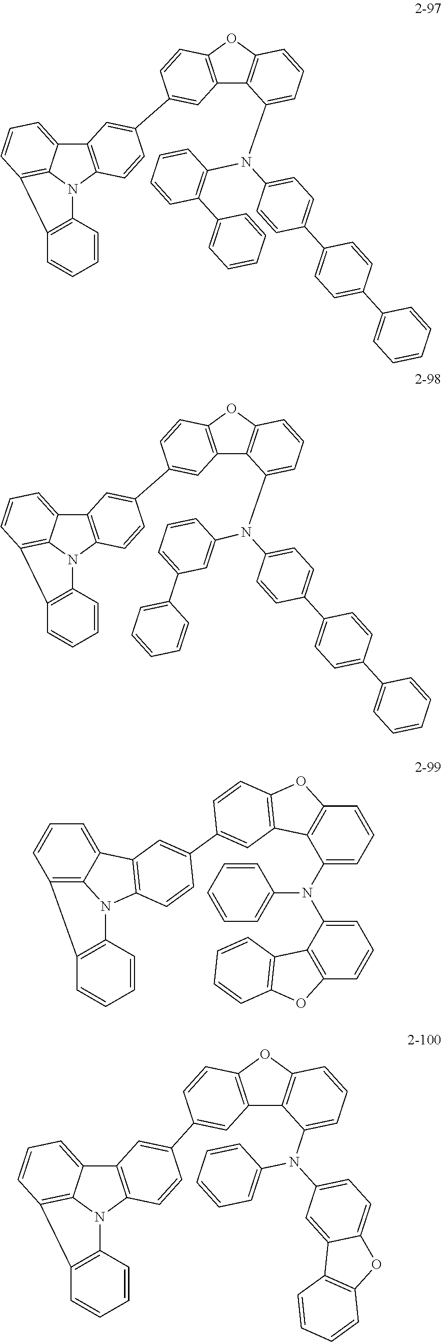 [SMILES: c1ccc(-c2ccc(-c3ccc(N(c4ccccc4-c4ccccc4)c4cccc5oc6ccc(-c7ccc8c(c7)c7cccc9c%10ccccc%10n8c97)cc6c45)cc3)cc2)cc1 ; c1ccc(-c2ccc(-c3ccc(N(c4cccc(-c5ccccc5)c4)c4cccc5oc6ccc(-c7ccc8c(c7)c7cccc9c%10ccccc%10n8c97)cc6c45)cc3)cc2)cc1 ; c1ccc(N(c2cccc3oc4ccccc4c23)c2cccc3oc4ccc(-c5ccc6c(c5)c5cccc7c8ccccc8n6c75)cc4c23)cc1 ; c1ccc(N(c2ccc3oc4ccccc4c3c2)c2cccc3oc4ccc(-c5ccc6c(c5)c5cccc7c8ccccc8n6c75)cc4c23)cc1]

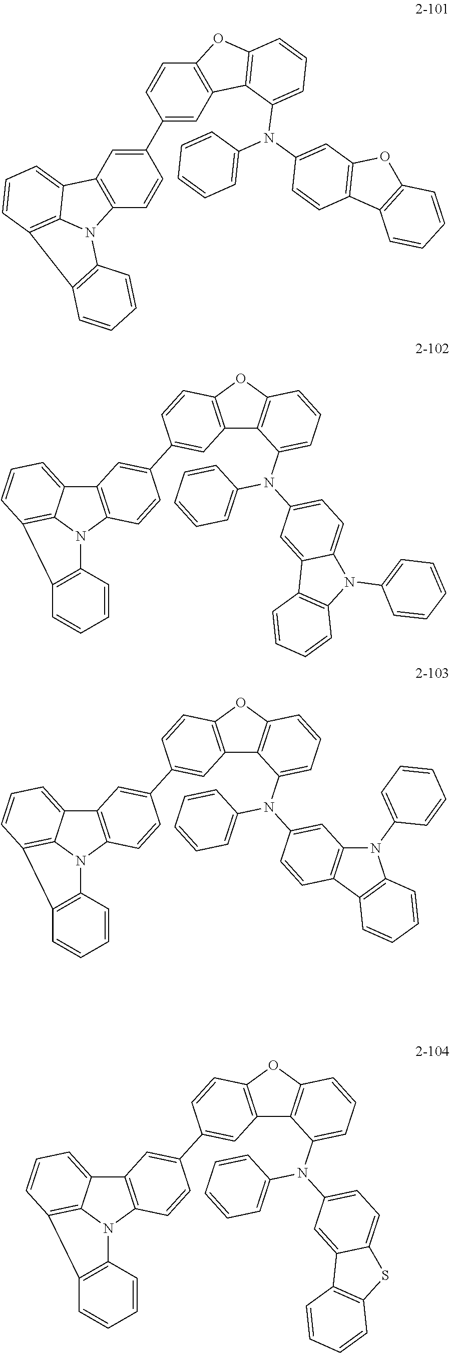 [SMILES: c1ccc(N(c2ccc3c(c2)oc2ccccc23)c2cccc3oc4ccc(-c5ccc6c(c5)c5cccc7c8ccccc8n6c75)cc4c23)cc1 ; c1ccc(N(c2ccc3c(c2)c2ccccc2n3-c2ccccc2)c2cccc3oc4ccc(-c5ccc6c(c5)c5cccc7c8ccccc8n6c75)cc4c23)cc1 ; c1ccc(N(c2ccc3c4ccccc4n(-c4ccccc4)c3c2)c2cccc3oc4ccc(-c5ccc6c(c5)c5cccc7c8ccccc8n6c75)cc4c23)cc1 ; c1ccc(N(c2ccc3sc4ccccc4c3c2)c2cccc3oc4ccc(-c5ccc6c(c5)c5cccc7c8ccccc8n6c75)cc4c23)cc1]

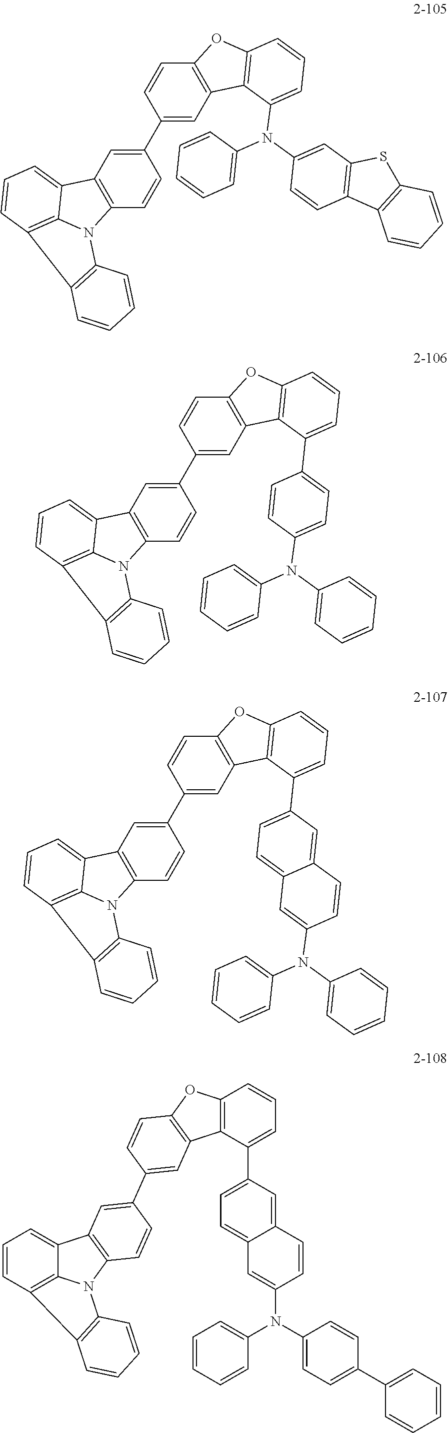 [SMILES: c1ccc(N(c2ccc3c(c2)sc2ccccc23)c2cccc3oc4ccc(-c5ccc6c(c5)c5cccc7c8ccccc8n6c75)cc4c23)cc1 ; c1ccc(N(c2ccccc2)c2ccc(-c3cccc4oc5ccc(-c6ccc7c(c6)c6cccc8c9ccccc9n7c86)cc5c34)cc2)cc1 ; c1ccc(N(c2ccccc2)c2ccc3cc(-c4cccc5oc6ccc(-c7ccc8c(c7)c7cccc9c%10ccccc%10n8c97)cc6c45)ccc3c2)cc1 ; c1ccc(-c2ccc(N(c3ccccc3)c3ccc4cc(-c5cccc6oc7ccc(-c8ccc9c(c8)c8cccc%10c%11ccccc%11n9c%108)cc7c56)ccc4c3)cc2)cc1]

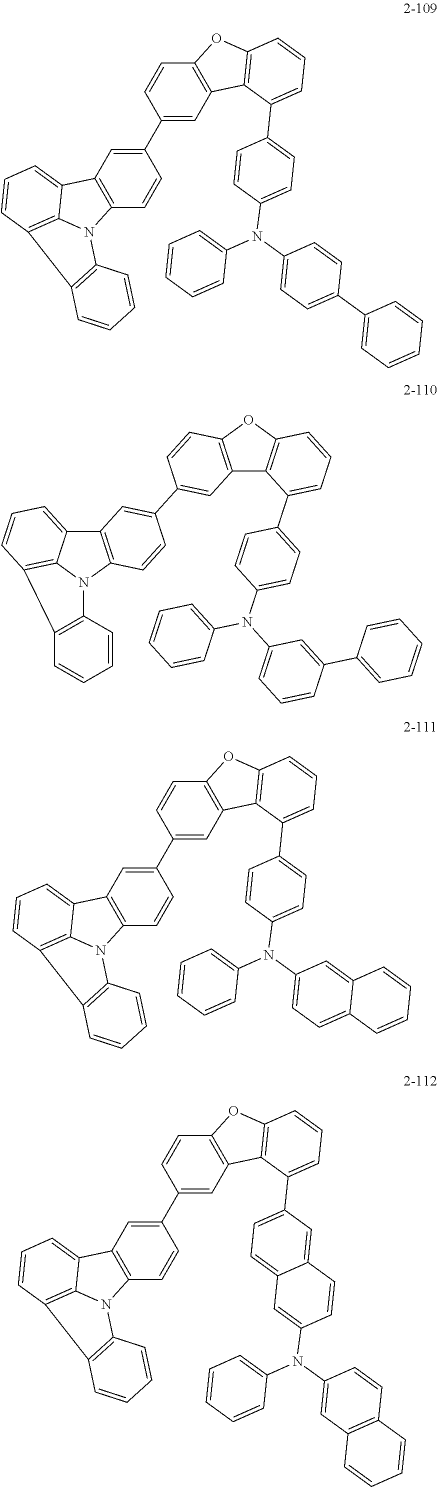 [SMILES: c1ccc(-c2ccc(N(c3ccccc3)c3ccc(-c4cccc5oc6ccc(-c7ccc8c(c7)c7cccc9c%10ccccc%10n8c97)cc6c45)cc3)cc2)cc1 ; c1ccc(-c2cccc(N(c3ccccc3)c3ccc(-c4cccc5oc6ccc(-c7ccc8c(c7)c7cccc9c%10ccccc%10n8c97)cc6c45)cc3)c2)cc1 ; c1ccc(N(c2ccc(-c3cccc4oc5ccc(-c6ccc7c(c6)c6cccc8c9ccccc9n7c86)cc5c34)cc2)c2ccc3ccccc3c2)cc1 ; c1ccc(N(c2ccc3ccccc3c2)c2ccc3cc(-c4cccc5oc6ccc(-c7ccc8c(c7)c7cccc9c%10ccccc%10n8c97)cc6c45)ccc3c2)cc1]

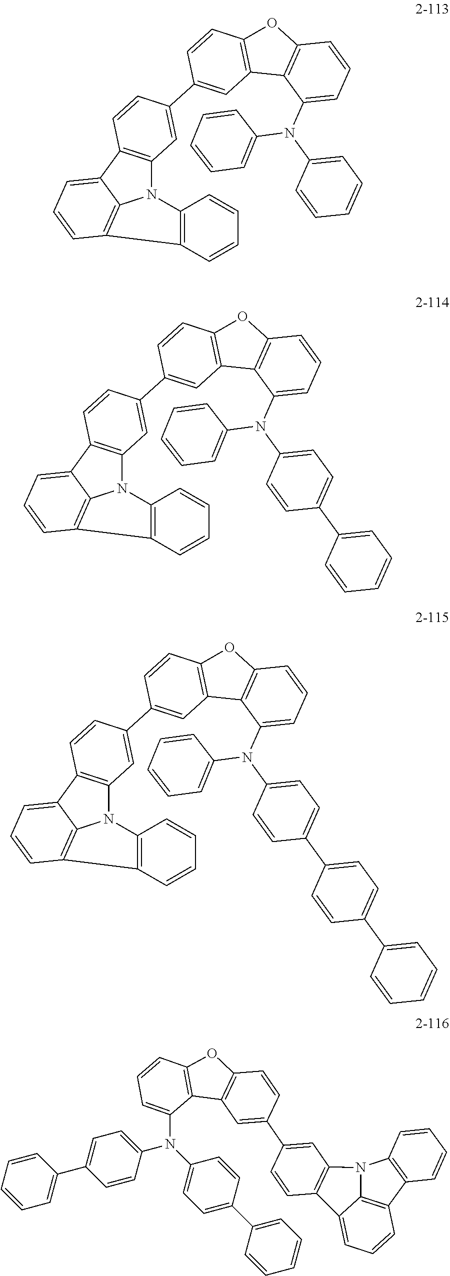 [SMILES: c1ccc(N(c2ccccc2)c2cccc3oc4ccc(-c5ccc6c7cccc8c9ccccc9n(c6c5)c87)cc4c23)cc1 ; c1ccc(-c2ccc(N(c3ccccc3)c3cccc4oc5ccc(-c6ccc7c8cccc9c%10ccccc%10n(c7c6)c98)cc5c34)cc2)cc1 ; c1ccc(-c2ccc(-c3ccc(N(c4ccccc4)c4cccc5oc6ccc(-c7ccc8c9cccc%10c%11ccccc%11n(c8c7)c%109)cc6c45)cc3)cc2)cc1 ; c1ccc(-c2ccc(N(c3ccc(-c4ccccc4)cc3)c3cccc4oc5ccc(-c6ccc7c8cccc9c%10ccccc%10n(c7c6)c98)cc5c34)cc2)cc1]

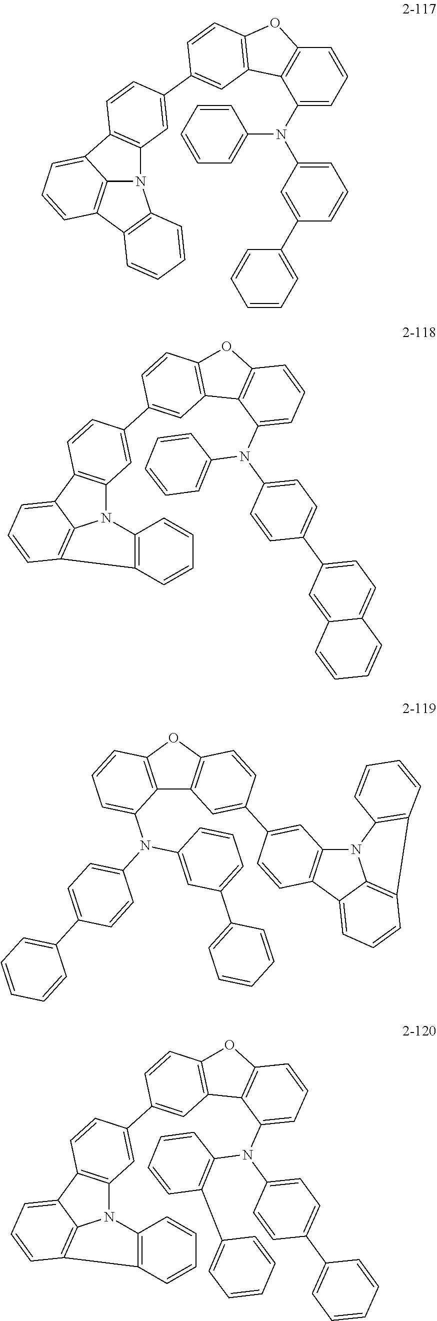

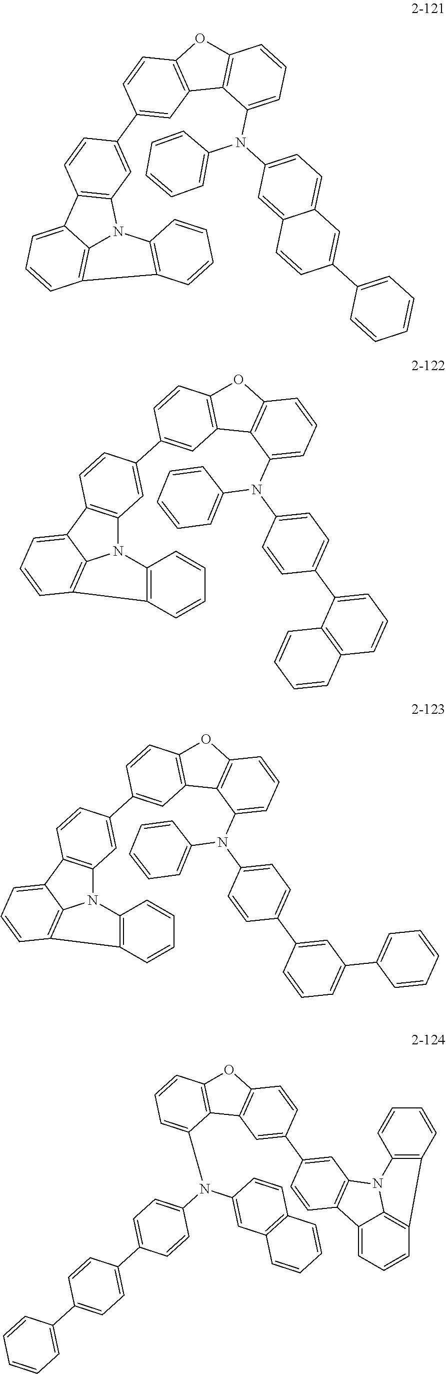 [SMILES: c1ccc(-c2ccc3cc(N(c4ccccc4)c4cccc5oc6ccc(-c7ccc8c9cccc%10c%11ccccc%11n(c8c7)c%109)cc6c45)ccc3c2)cc1 ; c1ccc(N(c2ccc(-c3cccc4ccccc34)cc2)c2cccc3oc4ccc(-c5ccc6c7cccc8c9ccccc9n(c6c5)c87)cc4c23)cc1 ; c1ccc(-c2cccc(-c3ccc(N(c4ccccc4)c4cccc5oc6ccc(-c7ccc8c9cccc%10c%11ccccc%11n(c8c7)c%109)cc6c45)cc3)c2)cc1 ; c1ccc(-c2ccc(-c3ccc(N(c4ccc5ccccc5c4)c4cccc5oc6ccc(-c7ccc8c9cccc%10c%11ccccc%11n(c8c7)c%109)cc6c45)cc3)cc2)cc1]

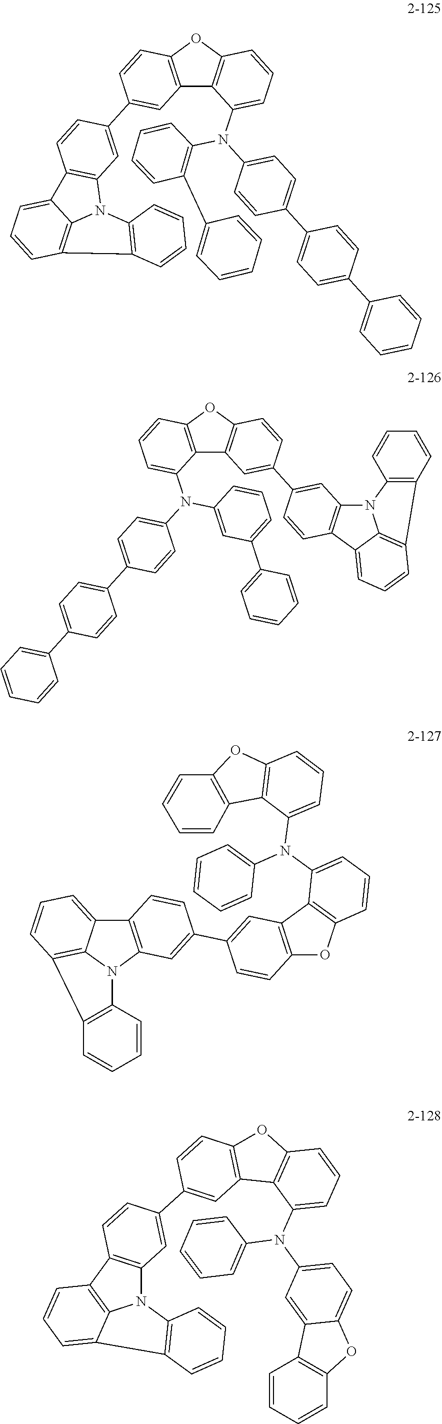 [SMILES: c1ccc(-c2ccc(-c3ccc(N(c4ccccc4-c4ccccc4)c4cccc5oc6ccc(-c7ccc8c9cccc%10c%11ccccc%11n(c8c7)c%109)cc6c45)cc3)cc2)cc1 ; c1ccc(-c2ccc(-c3ccc(N(c4cccc(-c5ccccc5)c4)c4cccc5oc6ccc(-c7ccc8c9cccc%10c%11ccccc%11n(c8c7)c%109)cc6c45)cc3)cc2)cc1 ; c1ccc(N(c2cccc3oc4ccccc4c23)c2cccc3oc4ccc(-c5ccc6c7cccc8c9ccccc9n(c6c5)c87)cc4c23)cc1 ; c1ccc(N(c2ccc3oc4ccccc4c3c2)c2cccc3oc4ccc(-c5ccc6c7cccc8c9ccccc9n(c6c5)c87)cc4c23)cc1]

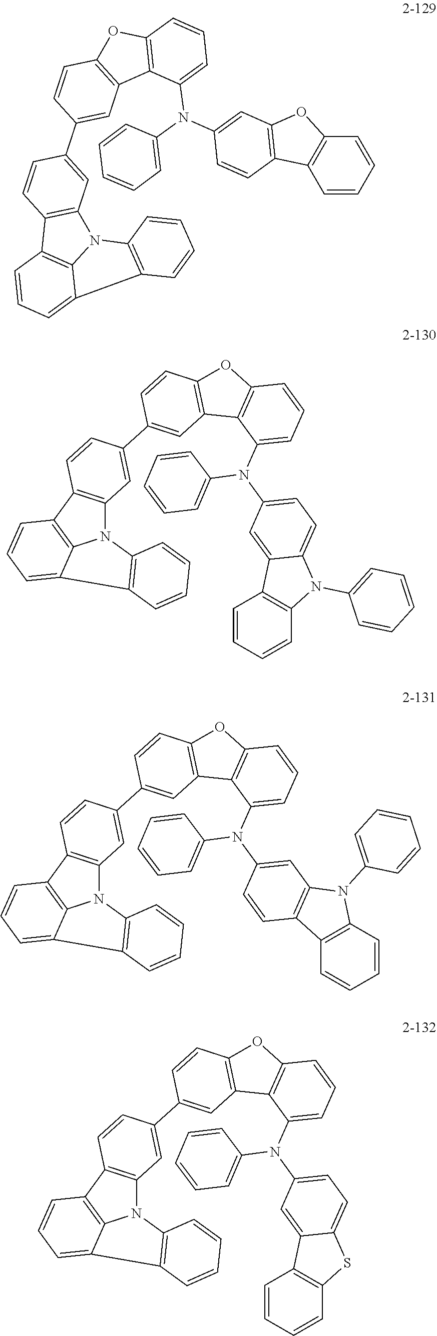 [SMILES: c1ccc(N(c2ccc3c(c2)oc2ccccc23)c2cccc3oc4ccc(-c5ccc6c7cccc8c9ccccc9n(c6c5)c87)cc4c23)cc1 ; c1ccc(N(c2ccc3c(c2)c2ccccc2n3-c2ccccc2)c2cccc3oc4ccc(-c5ccc6c7cccc8c9ccccc9n(c6c5)c87)cc4c23)cc1 ; c1ccc(N(c2ccc3c4ccccc4n(-c4ccccc4)c3c2)c2cccc3oc4ccc(-c5ccc6c7cccc8c9ccccc9n(c6c5)c87)cc4c23)cc1 ; c1ccc(N(c2ccc3sc4ccccc4c3c2)c2cccc3oc4ccc(-c5ccc6c7cccc8c9ccccc9n(c6c5)c87)cc4c23)cc1]

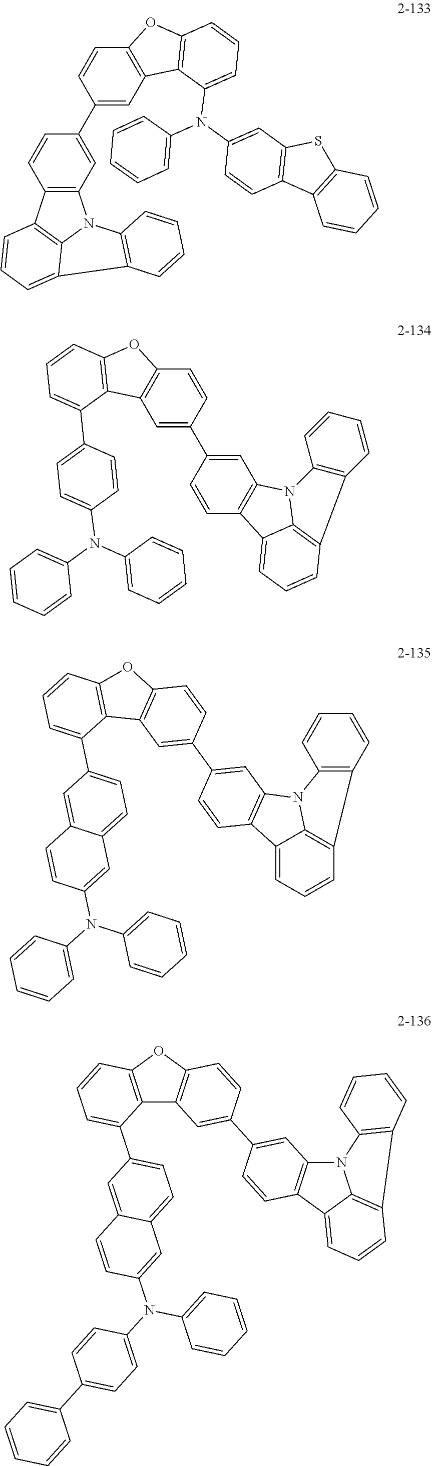 [SMILES: c1ccc(N(c2ccc3c(c2)sc2ccccc23)c2cccc3oc4ccc(-c5ccc6c7cccc8c9ccccc9n(c6c5)c87)cc4c23)cc1 ; c1ccc(N(c2ccccc2)c2ccc(-c3cccc4oc5ccc(-c6ccc7c8cccc9c%10ccccc%10n(c7c6)c98)cc5c34)cc2)cc1 ; c1ccc(N(c2ccccc2)c2ccc3cc(-c4cccc5oc6ccc(-c7ccc8c9cccc%10c%11ccccc%11n(c8c7)c%109)cc6c45)ccc3c2)cc1 ; c1ccc(-c2ccc(N(c3ccccc3)c3ccc4cc(-c5cccc6oc7ccc(-c8ccc9c%10cccc%11c%12ccccc%12n(c9c8)c%11%10)cc7c56)ccc4c3)cc2)cc1]

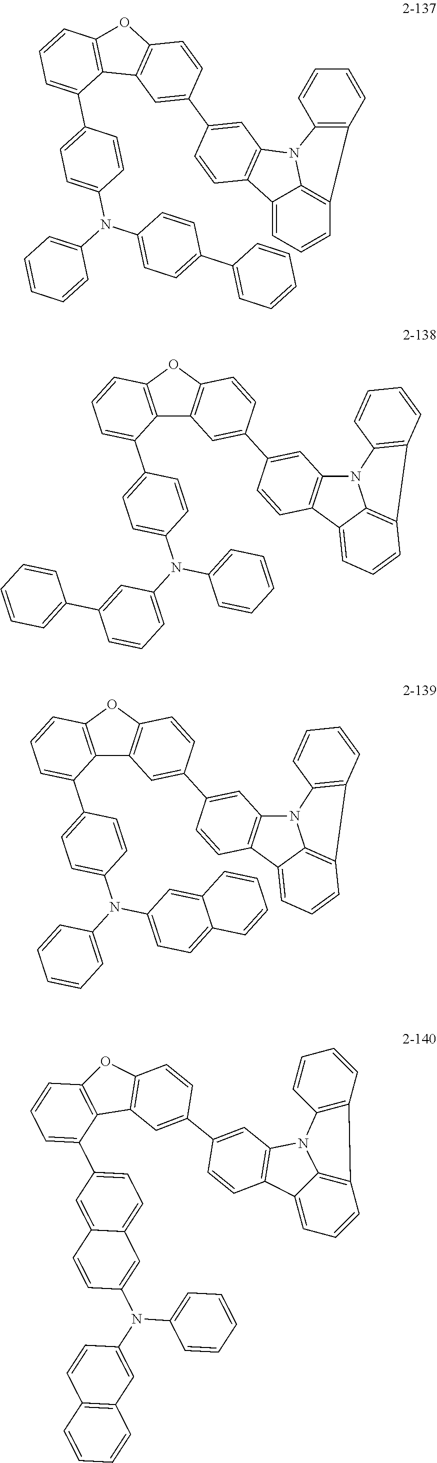 [SMILES: c1ccc(-c2ccc(N(c3ccccc3)c3ccc(-c4cccc5oc6ccc(-c7ccc8c9cccc%10c%11ccccc%11n(c8c7)c%109)cc6c45)cc3)cc2)cc1 ; c1ccc(-c2cccc(N(c3ccccc3)c3ccc(-c4cccc5oc6ccc(-c7ccc8c9cccc%10c%11ccccc%11n(c8c7)c%109)cc6c45)cc3)c2)cc1 ; c1ccc(N(c2ccc(-c3cccc4oc5ccc(-c6ccc7c8cccc9c%10ccccc%10n(c7c6)c98)cc5c34)cc2)c2ccc3ccccc3c2)cc1 ; c1ccc(N(c2ccc3ccccc3c2)c2ccc3cc(-c4cccc5oc6ccc(-c7ccc8c9cccc%10c%11ccccc%11n(c8c7)c%109)cc6c45)ccc3c2)cc1]

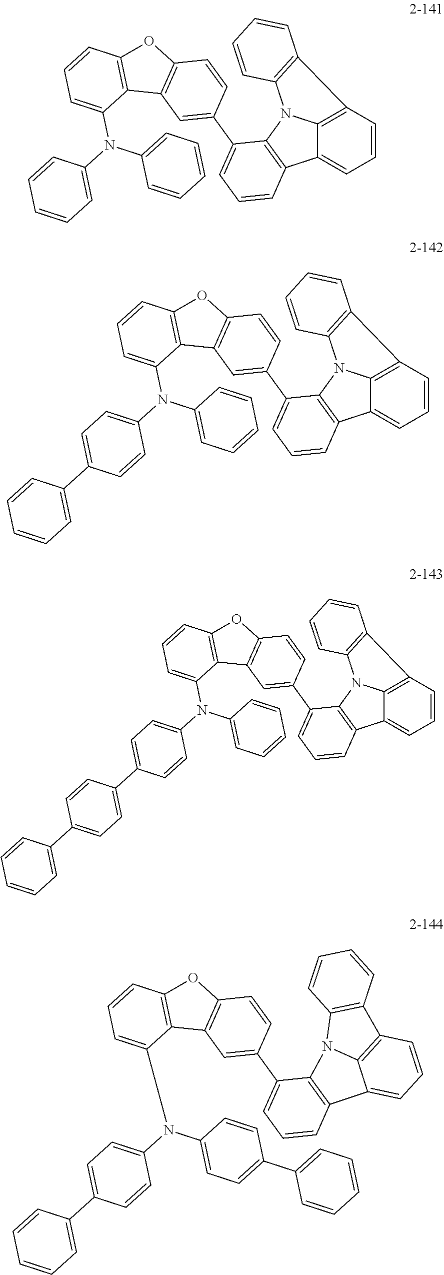 [SMILES: c1ccc(N(c2ccccc2)c2cccc3oc4ccc(-c5cccc6c7cccc8c9ccccc9n(c56)c87)cc4c23)cc1 ; c1ccc(-c2ccc(N(c3ccccc3)c3cccc4oc5ccc(-c6cccc7c8cccc9c%10ccccc%10n(c67)c98)cc5c34)cc2)cc1 ; c1ccc(-c2ccc(-c3ccc(N(c4ccccc4)c4cccc5oc6ccc(-c7cccc8c9cccc%10c%11ccccc%11n(c78)c%109)cc6c45)cc3)cc2)cc1 ; c1ccc(-c2ccc(N(c3ccc(-c4ccccc4)cc3)c3cccc4oc5ccc(-c6cccc7c8cccc9c%10ccccc%10n(c67)c98)cc5c34)cc2)cc1]

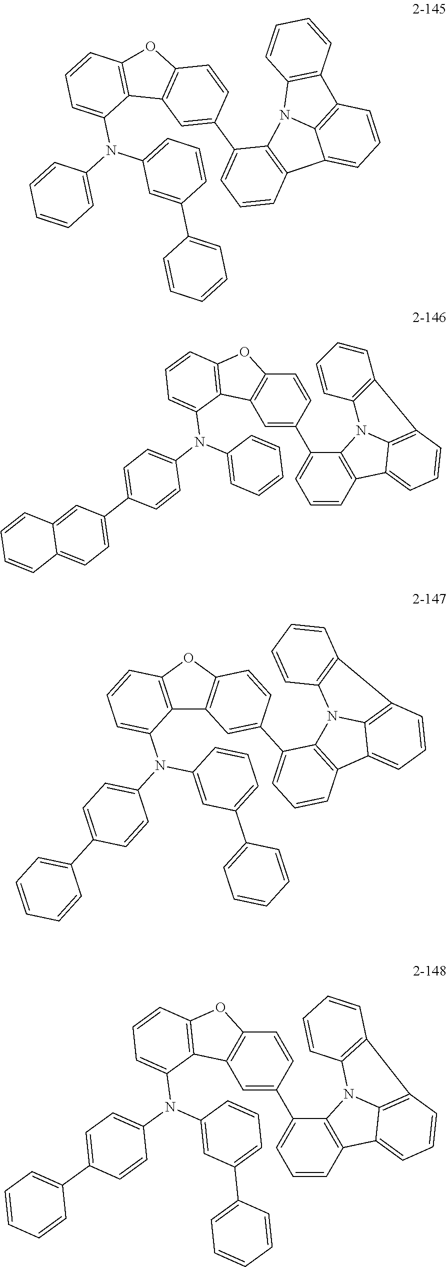 [SMILES: c1ccc(-c2cccc(N(c3ccccc3)c3cccc4oc5ccc(-c6cccc7c8cccc9c%10ccccc%10n(c67)c98)cc5c34)c2)cc1 ; c1ccc(N(c2ccc(-c3ccc4ccccc4c3)cc2)c2cccc3oc4ccc(-c5cccc6c7cccc8c9ccccc9n(c56)c87)cc4c23)cc1 ; c1ccc(-c2ccc(N(c3cccc(-c4ccccc4)c3)c3cccc4oc5ccc(-c6cccc7c8cccc9c%10ccccc%10n(c67)c98)cc5c34)cc2)cc1 ; c1ccc(-c2ccc(N(c3cccc(-c4ccccc4)c3)c3cccc4oc5ccc(-c6cccc7c8cccc9c%10ccccc%10n(c67)c98)cc5c34)cc2)cc1]

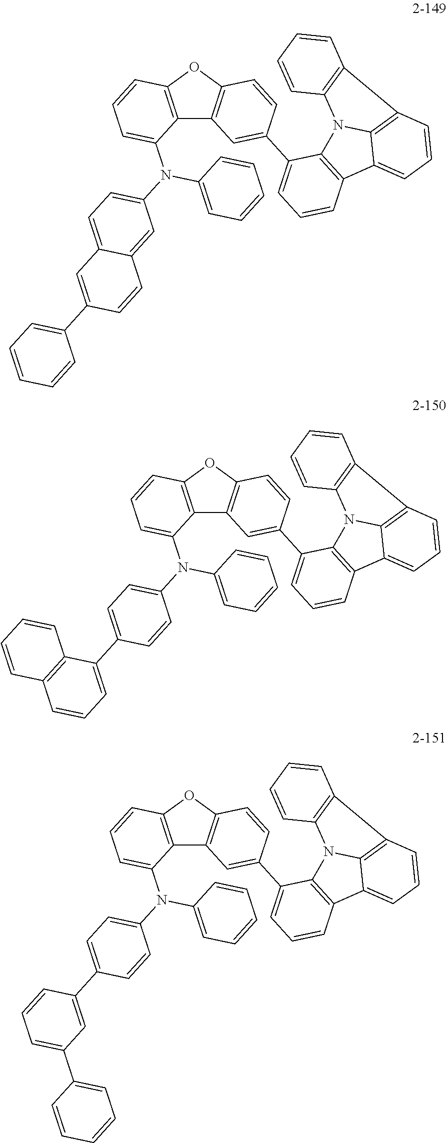 [SMILES: c1ccc(-c2ccc3cc(N(c4ccccc4)c4cccc5oc6ccc(-c7cccc8c9cccc%10c%11ccccc%11n(c78)c%109)cc6c45)ccc3c2)cc1 ; c1ccc(N(c2ccc(-c3cccc4ccccc34)cc2)c2cccc3oc4ccc(-c5cccc6c7cccc8c9ccccc9n(c56)c87)cc4c23)cc1 ; c1ccc(-c2cccc(-c3ccc(N(c4ccccc4)c4cccc5oc6ccc(-c7cccc8c9cccc%10c%11ccccc%11n(c78)c%109)cc6c45)cc3)c2)cc1]

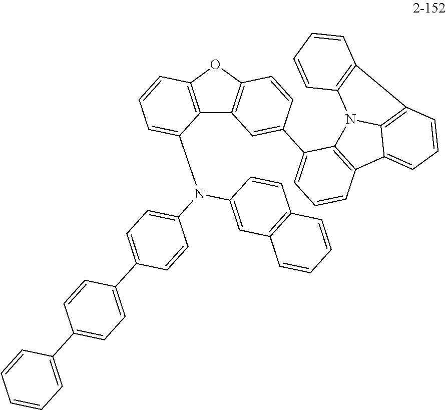 [SMILES: c1ccc(-c2ccc(-c3ccc(N(c4ccc5ccccc5c4)c4cccc5oc6ccc(-c7cccc8c9cccc%10c%11ccccc%11n(c78)c%109)cc6c45)cc3)cc2)cc1]

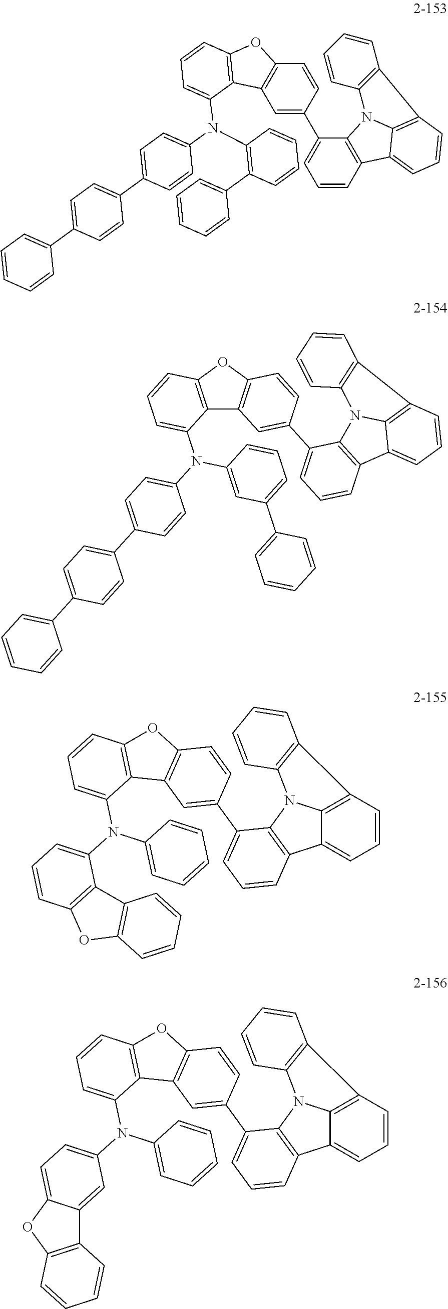 [SMILES: c1ccc(-c2ccc(-c3ccc(N(c4ccccc4-c4ccccc4)c4cccc5oc6ccc(-c7cccc8c9cccc%10c%11ccccc%11n(c78)c%109)cc6c45)cc3)cc2)cc1 ; c1ccc(-c2ccc(-c3ccc(N(c4cccc(-c5ccccc5)c4)c4cccc5oc6ccc(-c7cccc8c9cccc%10c%11ccccc%11n(c78)c%109)cc6c45)cc3)cc2)cc1 ; c1ccc(N(c2cccc3oc4ccccc4c23)c2cccc3oc4ccc(-c5cccc6c7cccc8c9ccccc9n(c56)c87)cc4c23)cc1 ; c1ccc(N(c2ccc3oc4ccccc4c3c2)c2cccc3oc4ccc(-c5cccc6c7cccc8c9ccccc9n(c56)c87)cc4c23)cc1]

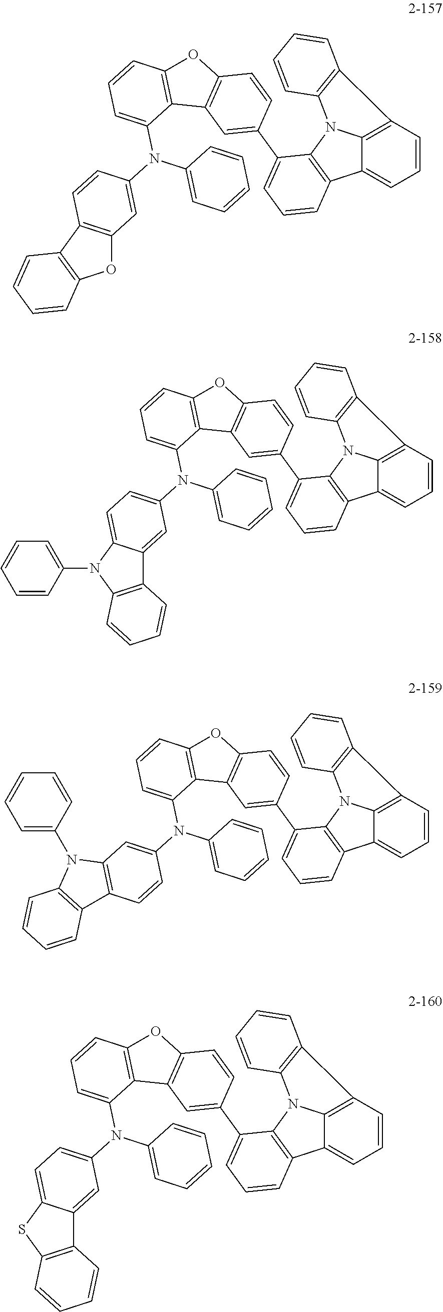 [SMILES: c1ccc(N(c2ccc3c(c2)oc2ccccc23)c2cccc3oc4ccc(-c5cccc6c7cccc8c9ccccc9n(c56)c87)cc4c23)cc1 ; c1ccc(N(c2ccc3c(c2)c2ccccc2n3-c2ccccc2)c2cccc3oc4ccc(-c5cccc6c7cccc8c9ccccc9n(c56)c87)cc4c23)cc1 ; c1ccc(N(c2ccc3c4ccccc4n(-c4ccccc4)c3c2)c2cccc3oc4ccc(-c5cccc6c7cccc8c9ccccc9n(c56)c87)cc4c23)cc1 ; c1ccc(N(c2ccc3sc4ccccc4c3c2)c2cccc3oc4ccc(-c5cccc6c7cccc8c9ccccc9n(c56)c87)cc4c23)cc1]

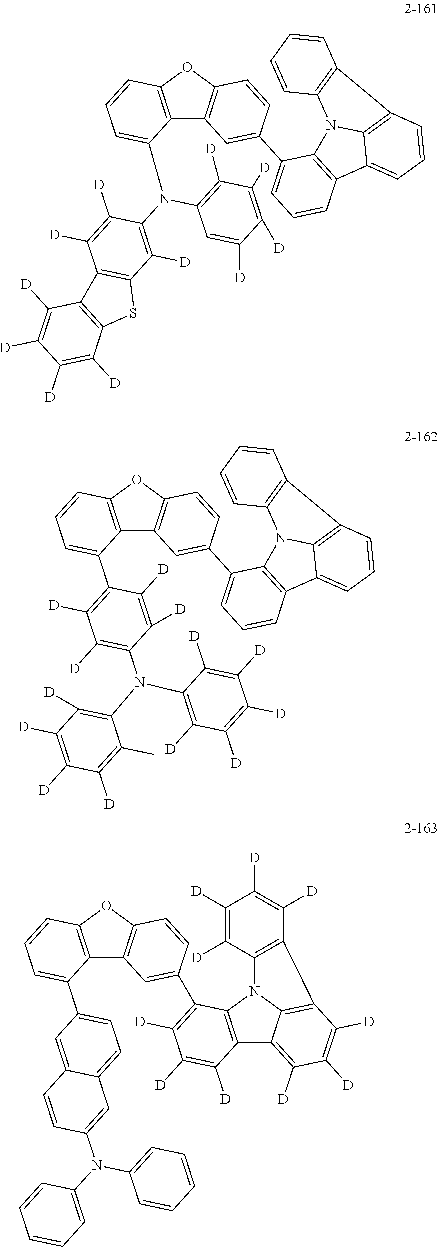 [SMILES: [2H]c1cc(N(c2c([2H])c([2H])c3c(sc4c([2H])c([2H])c([2H])c([2H])c43)c2[2H])c2cccc3oc4ccc(-c5cccc6c7cccc8c9ccccc9n(c56)c87)cc4c23)c([2H])c([2H])c1[2H] ; [2H]c1c([2H])c([2H])c(N(c2c([2H])c([2H])c(-c3cccc4oc5ccc(-c6cccc7c8cccc9c%10ccccc%10n(c67)c98)cc5c34)c([2H])c2[2H])c2c([2H])c([2H])c([2H])c([2H])c2C)c([2H])c1[2H] ; [2H]c1c([2H])c([2H])c2c(c1[2H])c1c([2H])c([2H])c([2H])c3c4c([2H])c([2H])c([2H])c(-c5ccc6oc7cccc(-c8ccc9cc(N(c%10ccccc%10)c%10ccccc%10)ccc9c8)c7c6c5)c4n2c13]

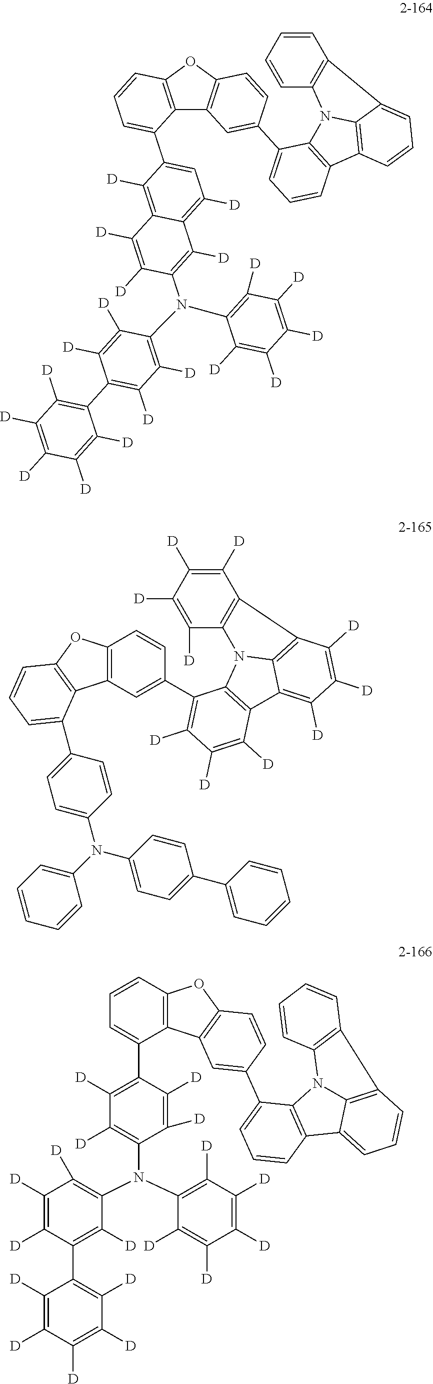

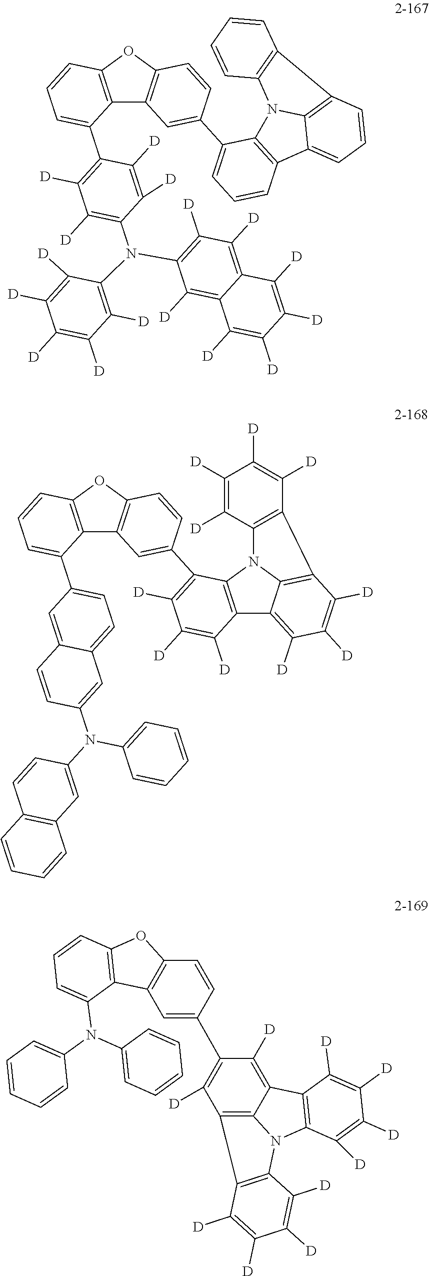 [SMILES: [2H]c1c([2H])c([2H])c(N(c2c([2H])c([2H])c(-c3cccc4oc5ccc(-c6cccc7c8cccc9c%10ccccc%10n(c67)c98)cc5c34)c([2H])c2[2H])c2c([2H])c([2H])c3c([2H])c([2H])c([2H])c([2H])c3c2[2H])c([2H])c1[2H] ; [2H]c1c([2H])c([2H])c2c(c1[2H])c1c([2H])c([2H])c([2H])c3c4c([2H])c([2H])c([2H])c(-c5ccc6oc7cccc(-c8ccc9cc(N(c%10ccccc%10)c%10ccc%11ccccc%11c%10)ccc9c8)c7c6c5)c4n2c13 ; [2H]c1c([2H])c([2H])c2c(c1[2H])c1c([2H])c(-c3ccc4oc5cccc(N(c6ccccc6)c6ccccc6)c5c4c3)c([2H])c3c4c([2H])c([2H])c([2H])c([2H])c4n2c13]

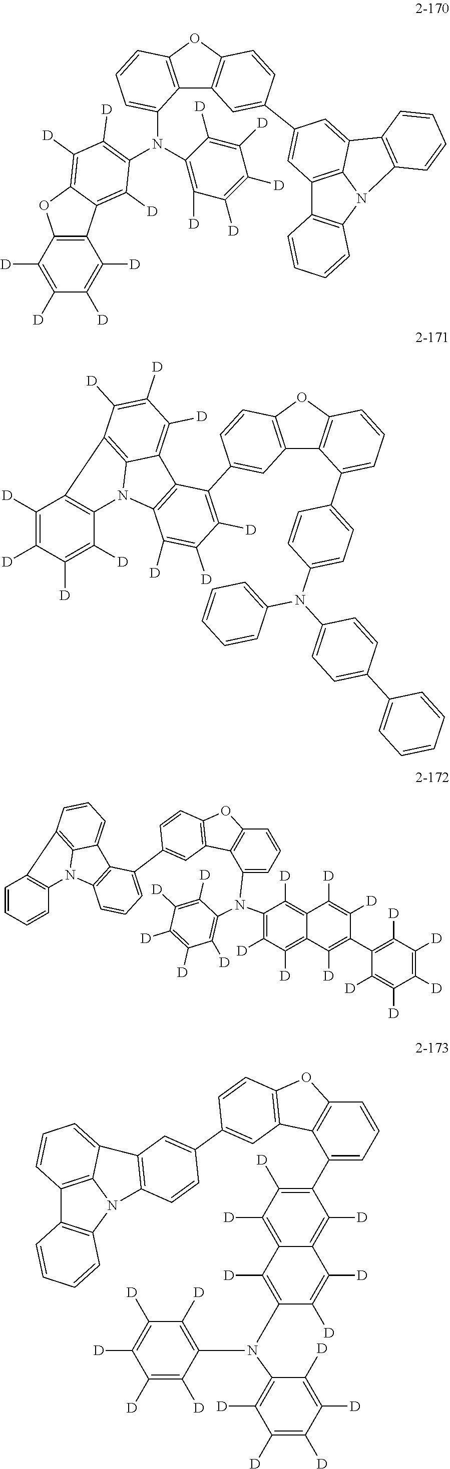

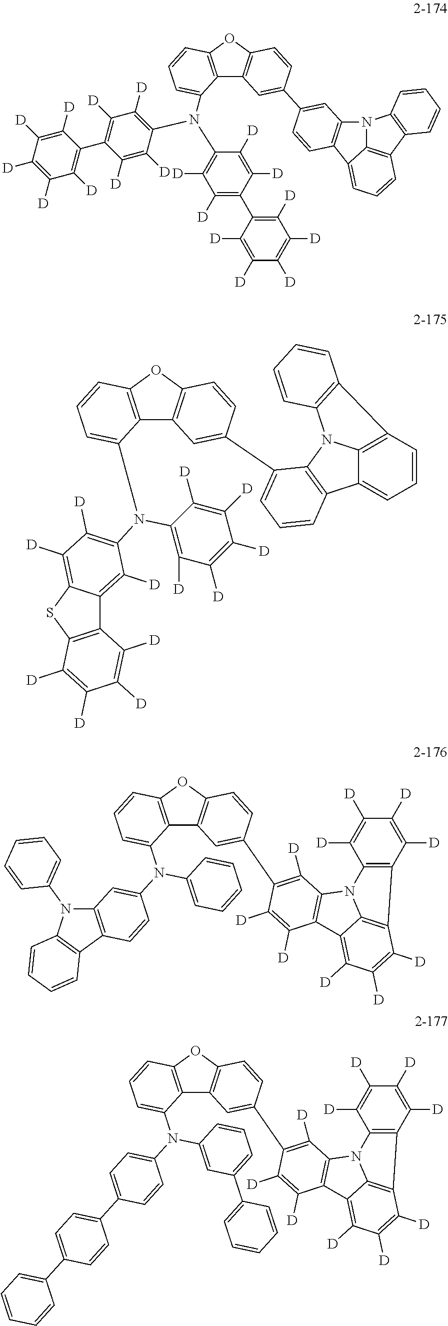

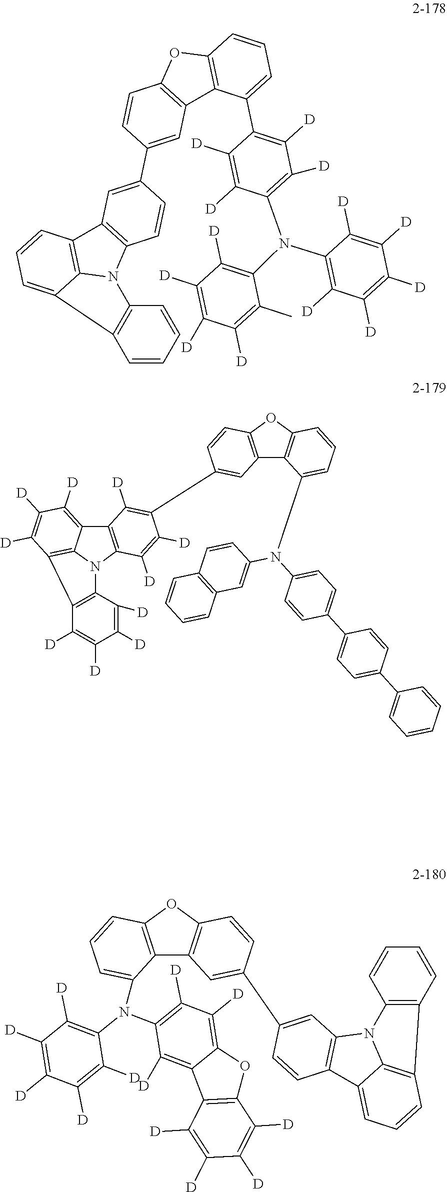 [SMILES: [2H]c1c([2H])c([2H])c(N(c2c([2H])c([2H])c(-c3cccc4oc5ccc(-c6ccc7c(c6)c6cccc8c9ccccc9n7c86)cc5c34)c([2H])c2[2H])c2c([2H])c([2H])c([2H])c([2H])c2C)c([2H])c1[2H] ; [2H]c1c([2H])c([2H])c2c(c1[2H])c1c([2H])c([2H])c([2H])c3c4c([2H])c(-c5ccc6oc7cccc(N(c8ccc(-c9ccc(-c%10ccccc%10)cc9)cc8)c8ccc9ccccc9c8)c7c6c5)c([2H])c([2H])c4n2c13 ; [2H]c1c([2H])c([2H])c(N(c2c([2H])c([2H])c3oc4c([2H])c([2H])c([2H])c([2H])c4c3c2[2H])c2cccc3oc4ccc(-c5ccc6c7cccc8c9ccccc9n(c6c5)c87)cc4c23)c([2H])c1[2H]]

In addition, by introducing various substituents to the structure of Chemical Formula 1, compounds having unique properties of the introduced substituents may be synthesized. For example, by introducing substituents normally used for a hole injection layer material, a hole transport layer material, a light emitting layer material, an electron transport layer material, an electron blocking layer material and a charge generation layer material used for manufacturing an organic light emitting device to the core structure, materials satisfying conditions required for each organic material layer may be synthesized.

In addition, by introducing various substituents to the structure of Chemical Formula 1, the energy band gap may be finely controlled, and meanwhile, properties at interfaces between organic materials may be enhanced, and material applications may become diverse.

Meanwhile, the heterocyclic compound has ahigh glass transition temperature (Tg), and thereby has excellent thermal stability. Such an increase in the thermal stability becomes an important factor providing driving stability to a device.

The heterocyclic compound according to one embodiment of the present disclosure may be prepared using a multi-step chemical reaction. Some intermediate compounds are prepared first, and from the intermediate compounds, the compound of Chemical Formula 1 may be prepared. More specifically, the heterocyclic compound according to one embodiment of the present disclosure may be prepared based on preparation examples to be described later.

Another embodiment of the present disclosure provides an organic light emitting device including the heterocyclic compound represented by Chemical Formula 1. The “organic light emitting device” may be expressed in terms such as an “organic light emitting diode”, an “OLED”, an “OLED device” and an “organic electroluminescent device”.

In addition, one embodiment of the present disclosure relates to an organic light emitting device comprising:
a first electrode;
a second electrode provided opposite to the first electrode; and one or more organic material layers provided between the first electrode and the second electrode,
wherein one or more layers of the one or more organic material layers comprise the heterocyclic compound represented by Chemical Formula 1.

In one embodiment of the present disclosure, the first electrode may be a positive electrode, and the second electrode may be a negative electrode.

In another embodiment, the first electrode may be a negative electrode, and the second electrode may be a positive electrode.

In one embodiment of the present disclosure, the organic light emitting device may be a red organic light emitting device, and the heterocyclic compound represented by Chemical Formula 1 may be used as a material of the red organic light emitting device.

In another embodiment of the present disclosure, the organic light emitting device may be a blue organic light emitting device, and the heterocyclic compound represented by Chemical Formula 1 may be used as a material of the blue organic light emitting device.

In another embodiment of the present disclosure, the organic light emitting device may be a green organic light emitting device, and the heterocyclic compound represented by Chemical Formula 1 may be used as a material of the green organic light emitting device.

In one embodiment of the present disclosure, the organic light emitting device may be a red organic light emitting device, and the heterocyclic compound represented by Chemical Formula 1 may be used as a light emitting layer material of the red organic light emitting device.

In another embodiment of the present disclosure, the organic light emitting device may be a blue organic light emitting device, and the heterocyclic compound represented by Chemical Formula 1 may be used as a light emitting layer material of the blue organic light emitting device.

In another embodiment of the present disclosure, the organic light emitting device may be a green organic light emitting device, and the heterocyclic compound represented by Chemical Formula 1 may be used as a light emitting layer material of the green organic light emitting device.

Specific descriptions on the heterocyclic compound represented by Chemical Formula 1 are the same as the descriptions provided above.

The organic light emitting device of the present disclosure may be manufactured using common organic light emitting device manufacturing methods and materials except that one or more organic material layers are formed using the heterocyclic compound described above.

The heterocyclic compound may be formed into the organic material layer using a solution coating method as well as a vacuum deposition method when the organic light emitting device is manufactured. Herein, the solution coating method means spin coating, dip coating, inkjet printing, screen printing, a spray method, roll coating and the like, but is not limited thereto.

The organic material layer of the organic light emitting device of the present disclosure may be formed in a single layer structure, but may also be formed in a multilayer structure in which two or more organic material layers are laminated. For example, the organic light emitting device of the present disclosure may have a structure including a hole injection layer, an electron blocking layer, a hole transport layer, a light emitting layer, an electron transport layer, a hole blocking layer, an electron injection layer and the like as the organic material layer. However, the structure of the organic light emitting device is not limited thereto, and may include a smaller number of organic material layers.

In the organic light emitting device of the present disclosure, the organic material layer includes a light emitting layer, and the light emitting layer may include the heterocyclic compound represented by Chemical Formula 1. When the heterocyclic compound is used in the light emitting layer, HOMO (Highest Occupied Molecular Orbital) and LUMO (Lowest Unoccupied Molecular Orbital) are spatially separated, enabling strong charge transfer, and therefore, driving efficiency and lifetime of the organic light emitting device may become superior.

One embodiment of the present disclosure provides an organic light emitting device, wherein the organic material layer comprising the heterocyclic compound represented by Chemical Formula 1 further comprises a heterocyclic compound represented by the following Chemical Formula 2.

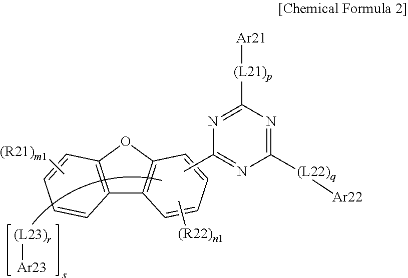

In Chemical Formula 2,
R21 and R22 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; —P(═O)R201R202; —SiR201R202R203; and —NR201R202, or two or more groups adjacent to each other bond to each other to form a substituted or unsubstituted C6 to C60 aromatic hydrocarbon ring; or a substituted or unsubstituted C2 to C60 heteroring, and R201, R202 and R203 are the same as or different from each other and each independently a substituted or unsubstituted C1 to C60 alkyl group; a substituted or unsubstituted C6 to C60 aryl group; or a substituted or unsubstituted C2 to C60 heteroaryl group,
m1 is an integer of 0 to 4, and when m1 is 2 or greater, R21s are the same as or different from each other,
n1 is an integer of 0 to 3, and when n1 is 2 or greater, R22s are the same as or different from each other,
L21 to L23 are the same as or different from each other, and each independently a direct bond; a substituted or unsubstituted C6 to C60 arylene group; or a substituted or unsubstituted C2 to C60 heteroarylene group,
p is an integer of 0 to 5, and when p is 2 or greater, L21s are the same as or different from each other,
q is an integer of 0 to 5, and when q is 2 or greater, L22s are the same as or different from each other,
r is an integer of 0 to 5, and when r is 2 or greater, L23s are the same as or different from each other,
Ar21 to Ar23 are the same as or different from each other, and each independently a substituted or unsubstituted C6 to C60 aryl group; or a substituted or unsubstituted C2 to C60 heteroaryl group, and
s is 0 or 1.

In one embodiment of the present disclosure, R21 and R22 are the same as or different from each other, and each independently hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; —P(═O)R201R202; —SiR201R202R203; or —NR201R202, or two or more groups adjacent to each other bond to each other to form a substituted or unsubstituted C6 to C30 aromatic hydrocarbon ring; or a substituted or unsubstituted C2 to C30 heteroring, and R201, R202 and R203 are the same as or different from each other and may be each independently a substituted or unsubstituted C1 to C30 alkyl group; a substituted or unsubstituted C6 to C30 aryl group; or a substituted or unsubstituted C2 to C30 heteroaryl group.

In another embodiment of the present disclosure, R21 and R22 are the same as or different from each other, and each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; —P(═O)R201R202; —SiR201R202R203; or —NR201R202, or two or more groups adjacent to each other bond to each other to form a substituted or unsubstituted C6 to C20 aromatic hydrocarbon ring; or a substituted or unsubstituted C2 to C20 heteroring, and R201, R202 and R203 are the same as or different from each other and may be each independently a substituted or unsubstituted C1 to C20 alkyl group; a substituted or unsubstituted C6 to C20 aryl group; or a substituted or unsubstituted C2 to C20 heteroaryl group.

In another embodiment of the present disclosure, R21 and R22 are the same as or different from each other, and may be each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; or a substituted or unsubstituted C2 to C20 heterocycloalkyl group.

In another embodiment of the present disclosure, R21 and R22 are the same as or different from each other, and may be each independently hydrogen; or deuterium.

In one embodiment of the present disclosure, L21 to L23 are the same as or different from each other, and may be each independently a direct bond; a substituted or unsubstituted C6 to C30 arylene group; or a substituted or unsubstituted C2 to C30 heteroarylene group.

In another embodiment of the present disclosure, L21 to L23 are the same as or different from each other, and may be each independently a direct bond; a substituted or unsubstituted C6 to C20 arylene group; or a substituted or unsubstituted C2 to C20 heteroarylene group.

In another embodiment of the present disclosure, L21 and L22 are the same as or different from each other, and may be each independently a direct bond; a substituted or unsubstituted phenylene group; a substituted or unsubstituted biphenylene group; a substituted or unsubstituted naphthylene group; a substituted or unsubstituted dibenzothiophenylene group; a substituted or unsubstituted dibenzofuranylene group; or a substituted or unsubstituted carbazolene group.

In another embodiment of the present disclosure, L23 may be a direct bond; a substituted or unsubstituted phenylene group; a substituted or unsubstituted biphenylene group; a substituted or unsubstituted naphthylene group; or a substituted or unsubstituted carbazolene group.

In one embodiment of the present disclosure, Ar21 to Ar23 are the same as or different from each other, and may be each independently a substituted or unsubstituted C6 to C30 aryl group; or a substituted or unsubstituted C2 to C30 heteroaryl group.

In another embodiment of the present disclosure, Ar21 to Ar23 are the same as or different from each other, and may be each independently a substituted or unsubstituted C6 to C20 aryl group; or a substituted or unsubstituted C2 to C20 heteroaryl group.

In another embodiment of the present disclosure, Ar21 and Ar22 are the same as or different from each other, and may be each independently a substituted or unsubstituted phenyl group; a substituted or unsubstituted biphenyl group; a substituted or unsubstituted terphenyl group; a substituted or unsubstituted naphthyl group; a substituted or unsubstituted dibenzofuranyl group; a substituted or unsubstituted benzonaphthofuranyl group; a substituted or unsubstituted dibenzothiophenyl group; or a substituted or unsubstituted carbazolyl group.

In another embodiment of the present disclosure, Ar23 may be a substituted or unsubstituted phenyl group; a substituted or unsubstituted biphenyl group; a substituted or unsubstituted terphenyl group; a substituted or unsubstituted naphthyl group; a substituted or unsubstituted dibenzofuranyl group; a substituted or unsubstituted benzonaphthofuranyl group; a substituted or unsubstituted dibenzothiophenyl group; or a substituted or unsubstituted carbazolyl group.

In one embodiment of the present disclosure, the heterocyclic compound represented by Chemical Formula 2 may be represented by any one of the following Chemical Formulae 2-1 to 2-3.

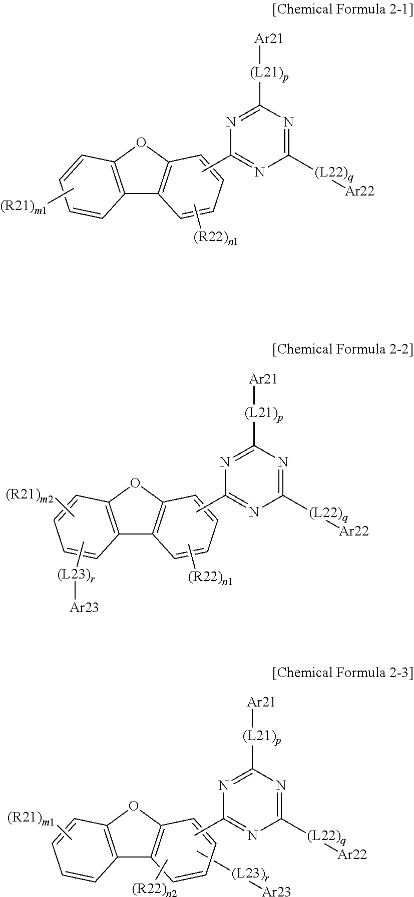

In Chemical Formulae 2-1 to 2-3,
m2 is an integer of 0 to 3, and when m2 is 2 or greater, R21s are the same as or different from each other,
n2 is an integer of 0 to 2, and when n2 is 2 or greater, R22s are the same as or different from each other, and
R21, R22, L21 to L23, Ar21 to Ar23, m1, n1 and p to r have the same definitions as in Chemical Formula 2.

In one embodiment of the present disclosure, R21, R22, L21 to L23 and Ar21 to Ar23 may all include hydrogen (H) that is not deuterated.

In another embodiment of the present disclosure, at least one of R21, R22, L21 to L23 and Ar21 to Ar23 includes deuterium (D), and at least one of R21, R22, L21 to L23 and Ar21 to Ar23 may include hydrogen that is not deuterated.

In another embodiment of the present disclosure, R21, R22, L21 to L23 and Ar2 to Ar23 may all include deuterium.

In one embodiment of the present disclosure, the heterocyclic compound represented by Chemical Formula 2 may not include deuterium as a substituent, or may have a deuterium content of, for example, greater than 0%, 1% or greater, 10% or greater, 20% or greater, 30% or greater, 40% or greater or 50% or greater, and 100% or less, 90% or less, 80% or less, 70% or less or 60% or less with respect to the total number of hydrogen atoms and deuterium atoms.

In another embodiment of the present disclosure, the heterocyclic compound represented by Chemical Formula 2 may not include deuterium as a substituent, or may have a deuterium content of 1% to 100% with respect to the total number of hydrogen atoms and deuterium atoms.

In another embodiment of the present disclosure, the heterocyclic compound represented by Chemical Formula 2 may not include deuterium as a substituent, or may have a deuterium content of 20% to 90% with respect to the total number of hydrogen atoms and deuterium atoms.

In another embodiment of the present disclosure, the heterocyclic compound represented by Chemical Formula 2 may not include deuterium as a substituent, or may have a deuterium content of 30% to 80% with respect to the total number of hydrogen atoms and deuterium atoms.

In another embodiment of the present disclosure, the heterocyclic compound represented by Chemical Formula 2 may not include deuterium as a substituent, or may have a deuterium content of 50% to 70% with respect to the total number of hydrogen atoms and deuterium atoms.

In one embodiment of the present disclosure, the heterocyclic compound represented by Chemical Formula 2 may be represented by any one of the following compounds.

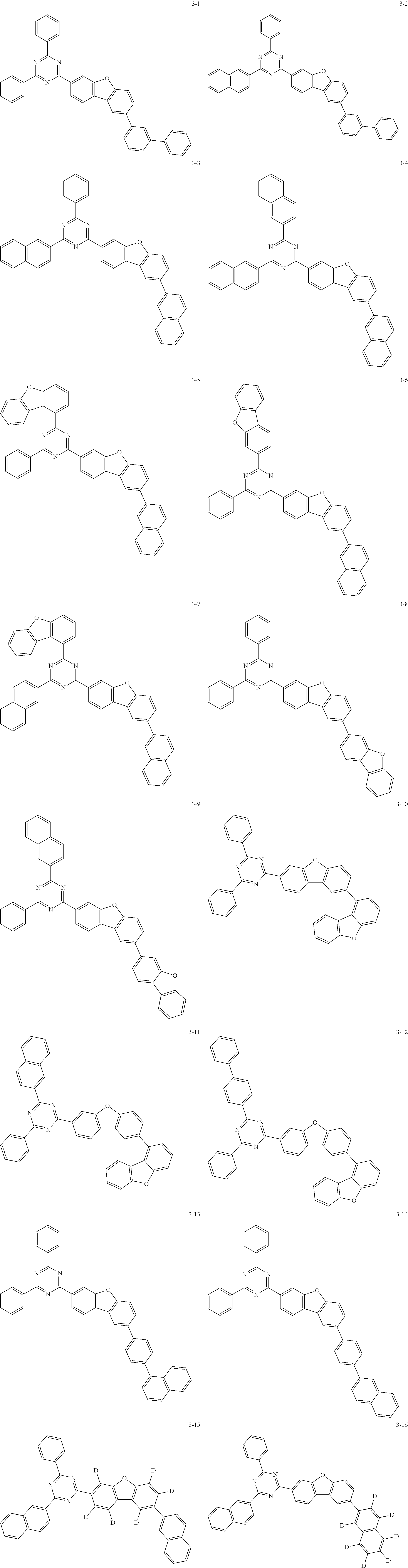

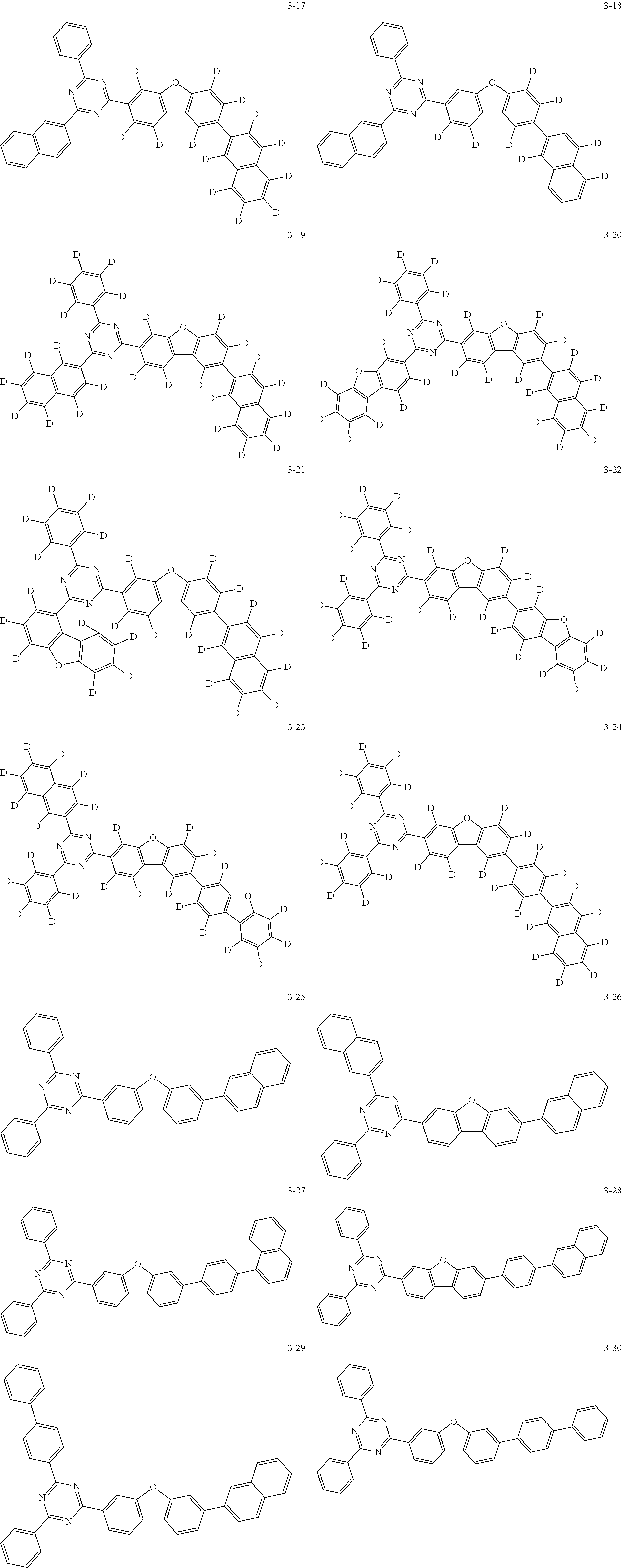

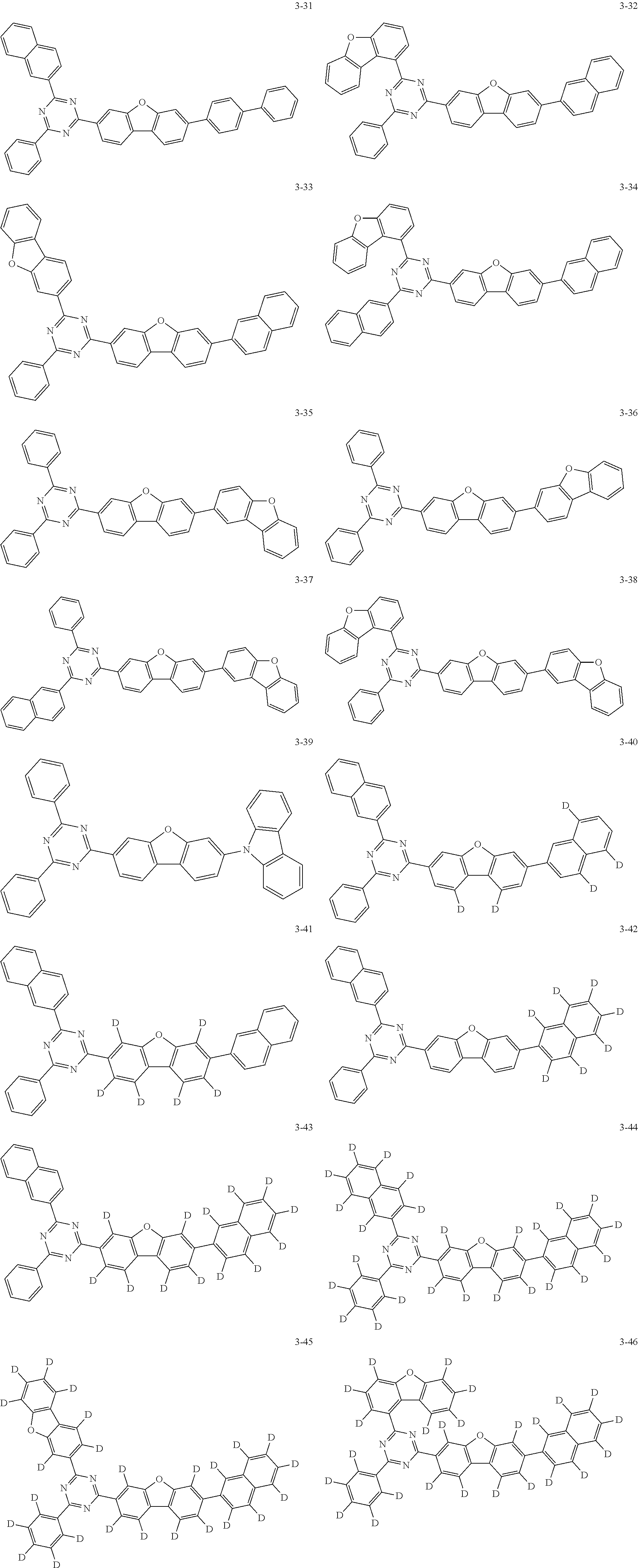

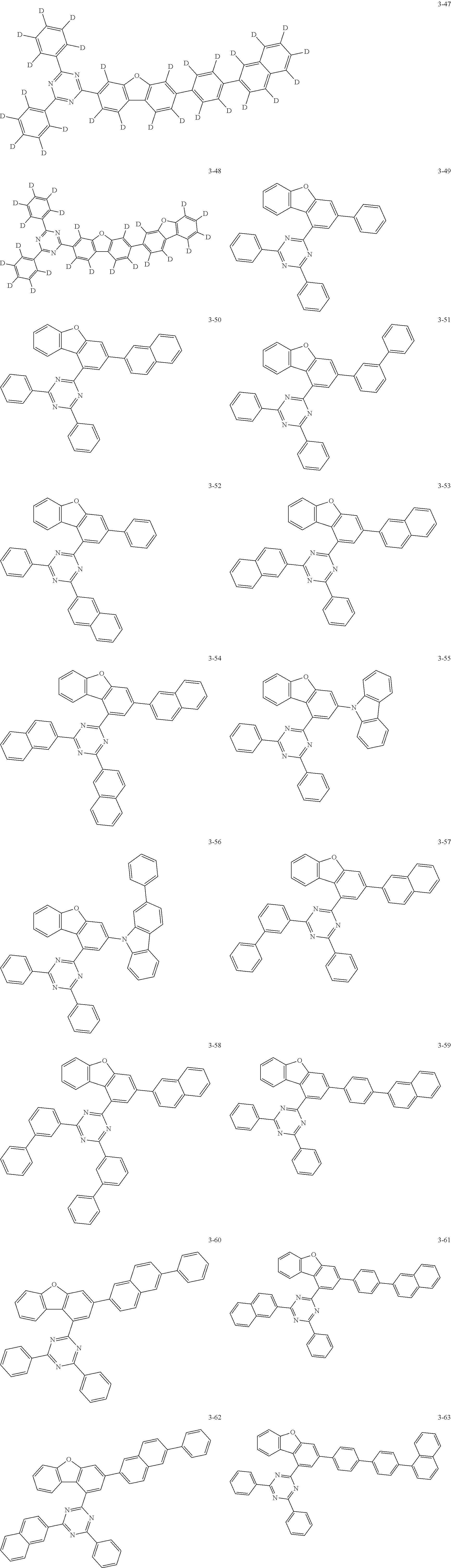

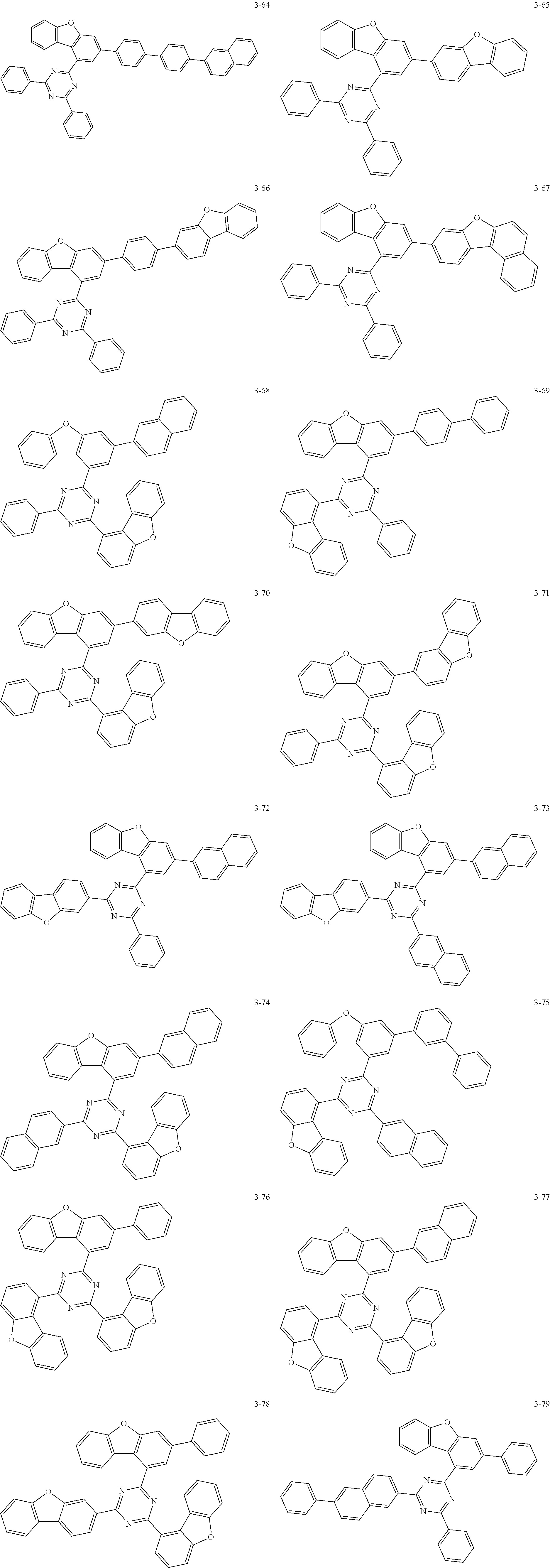

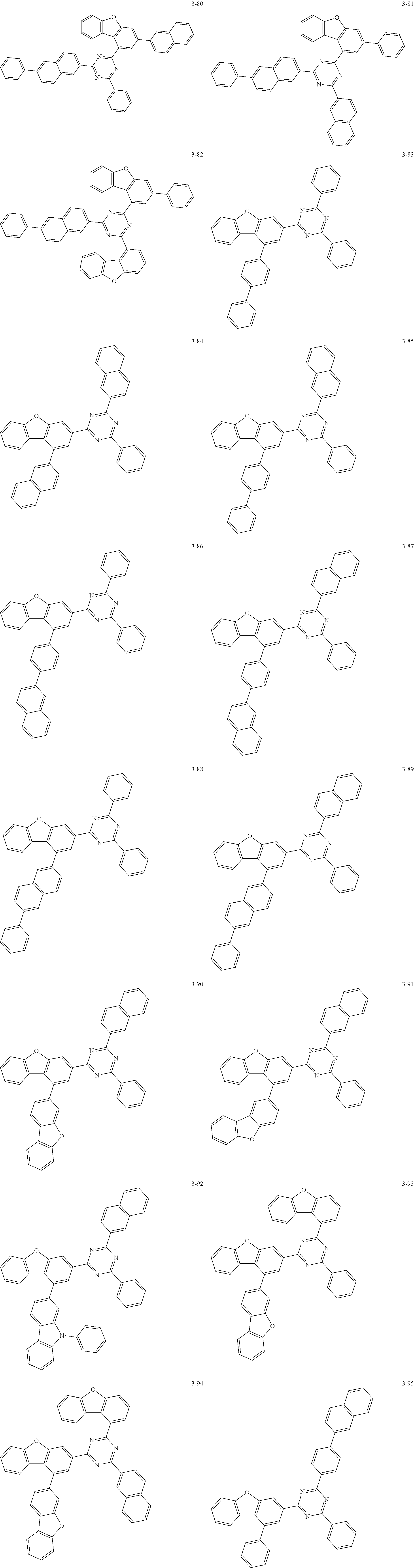

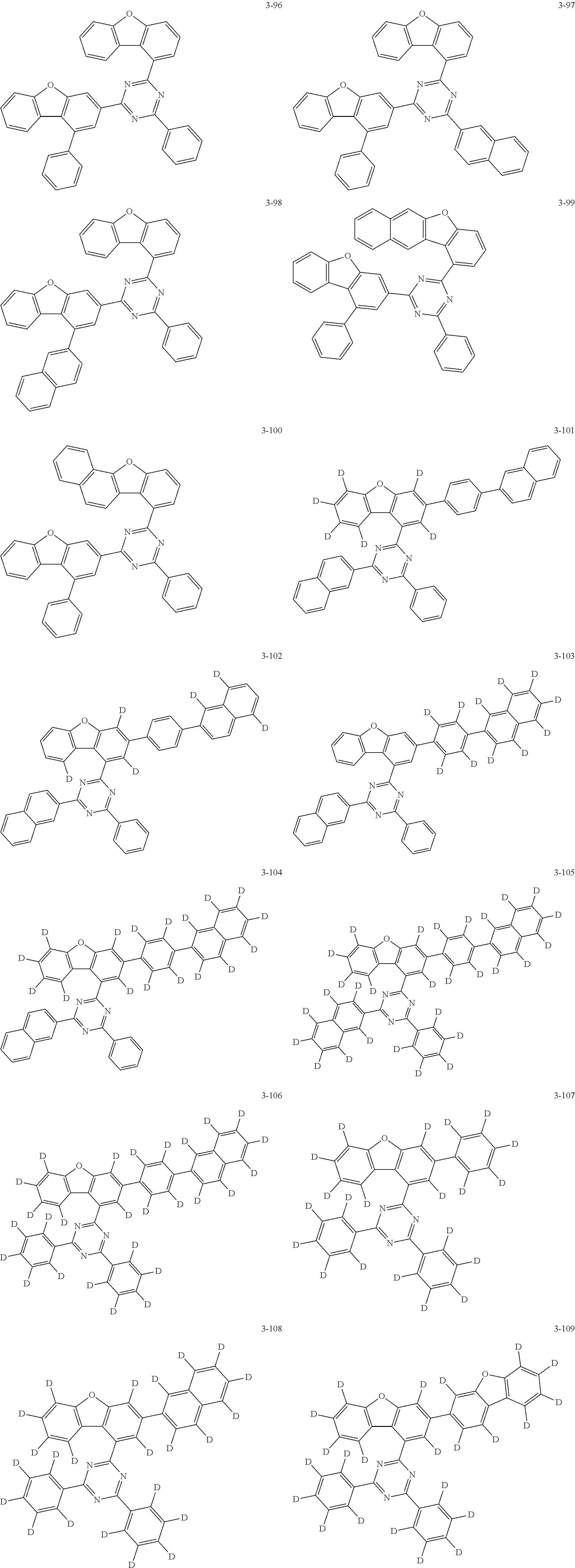

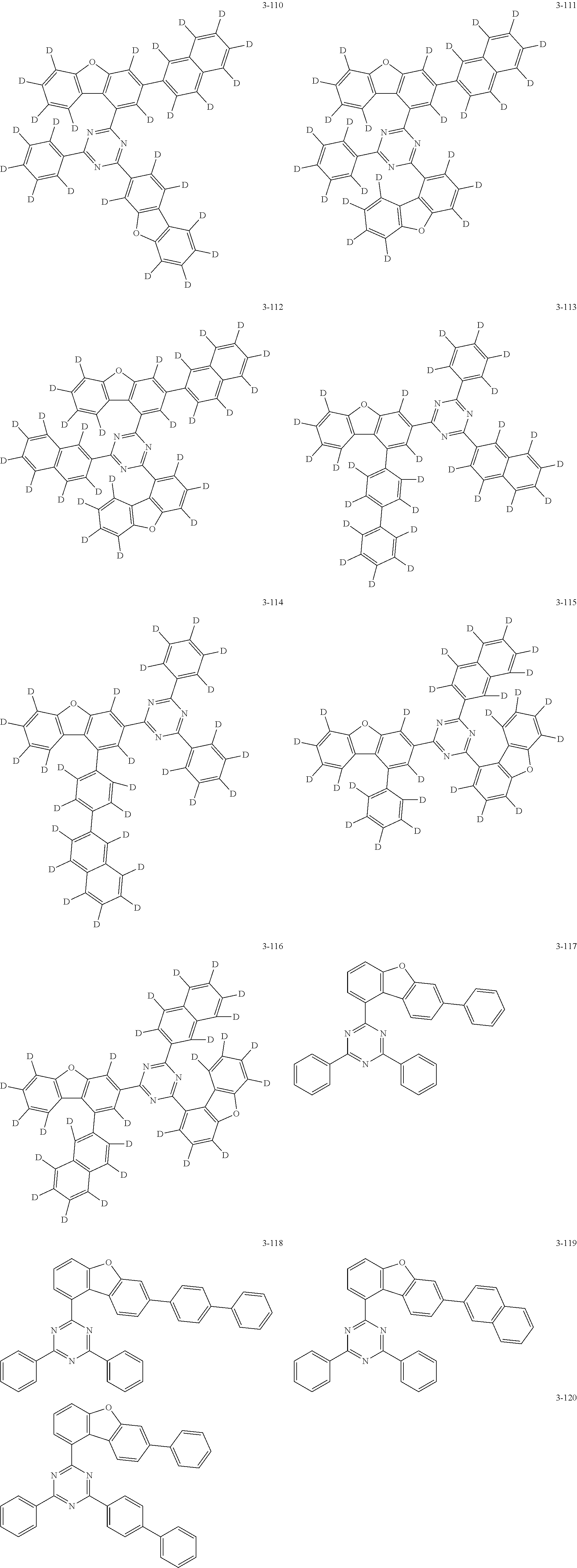

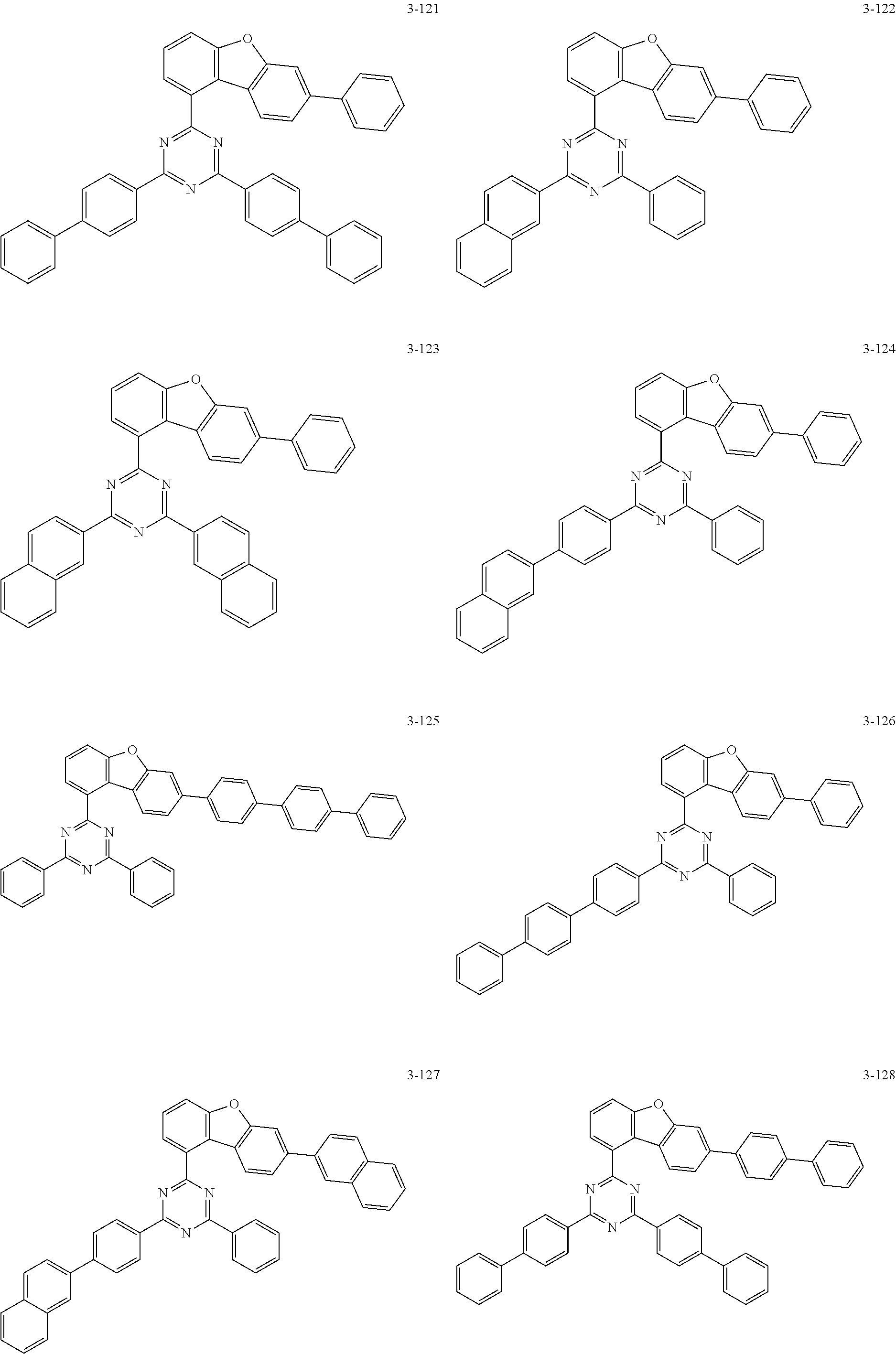

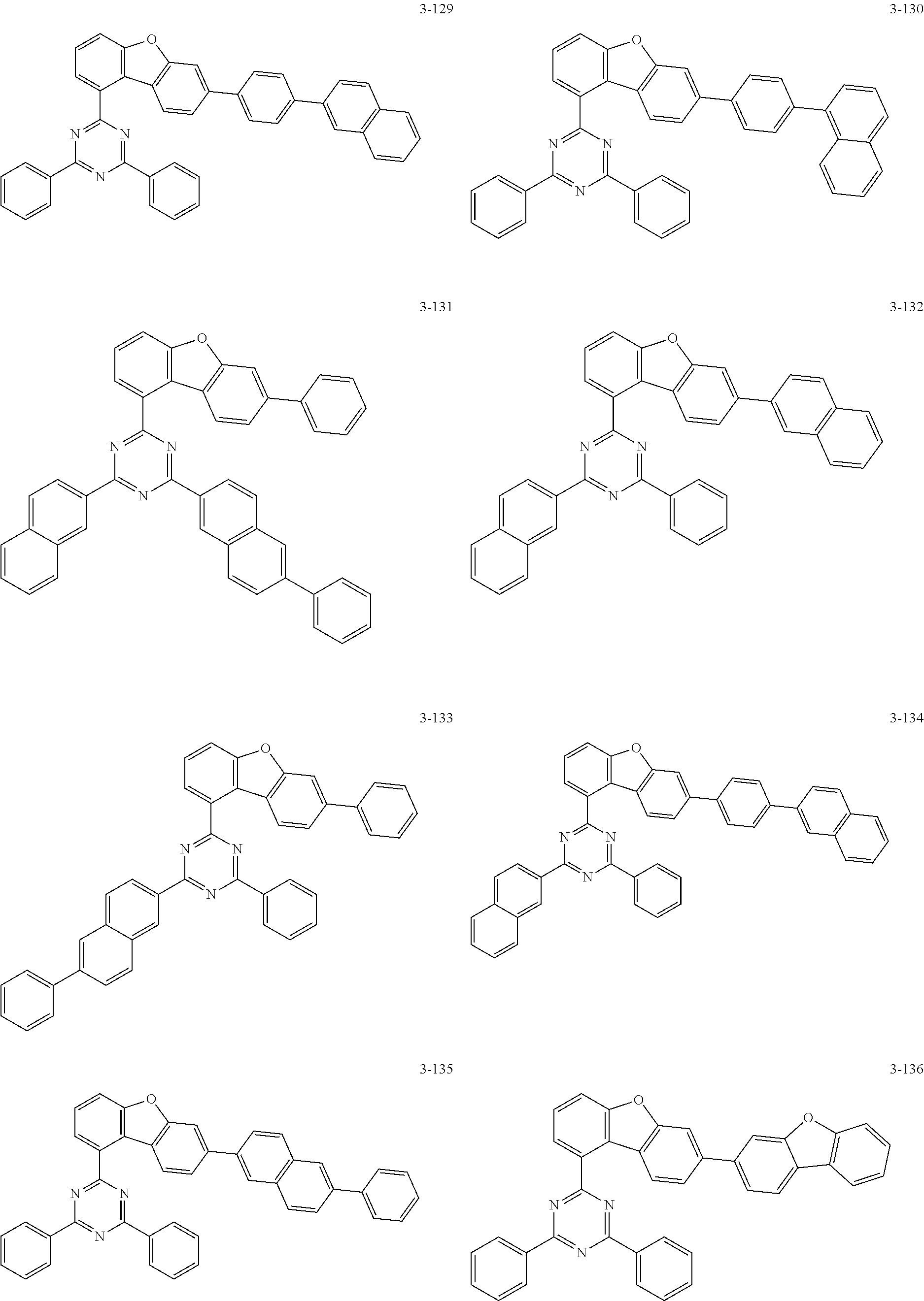

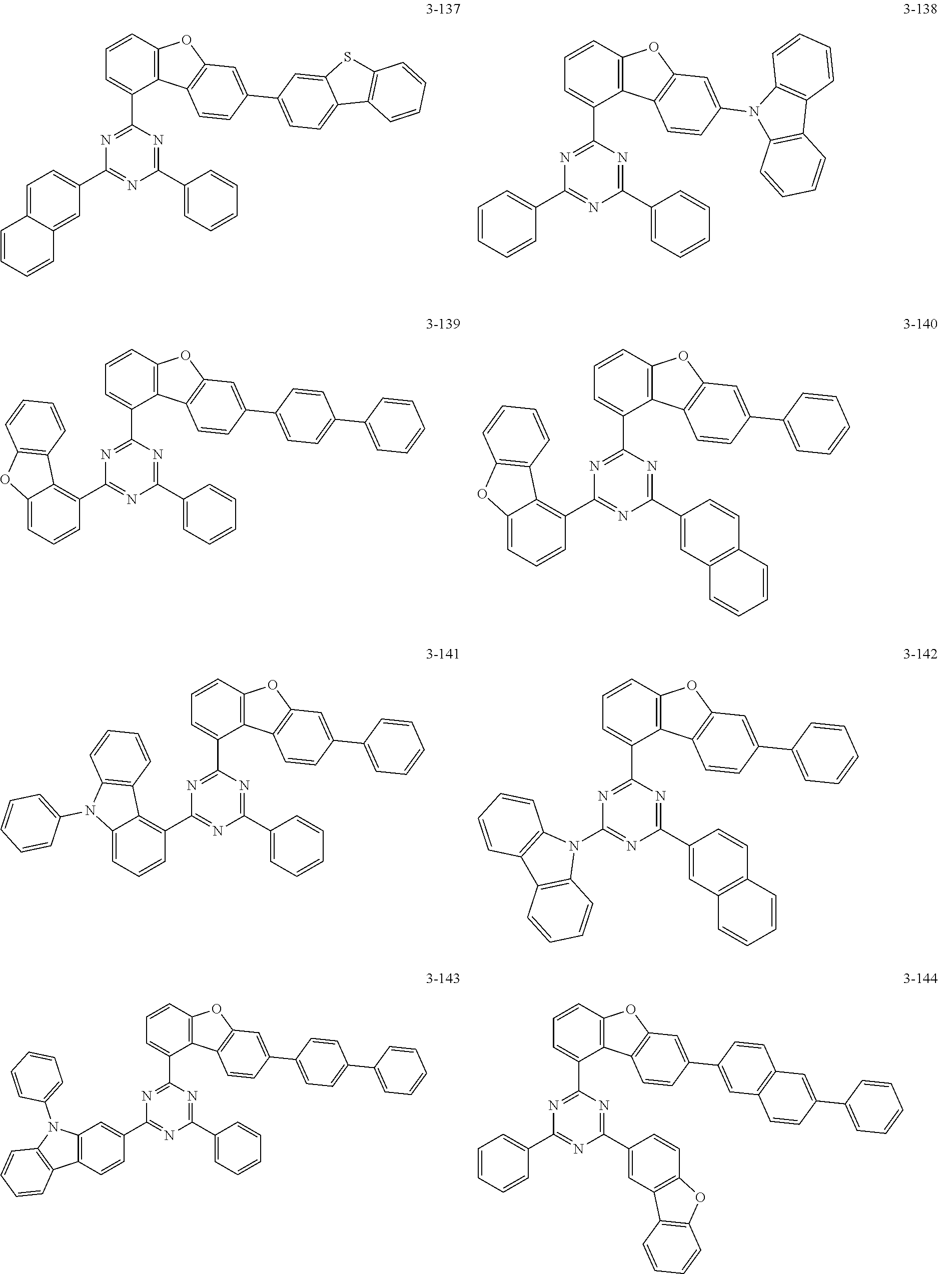

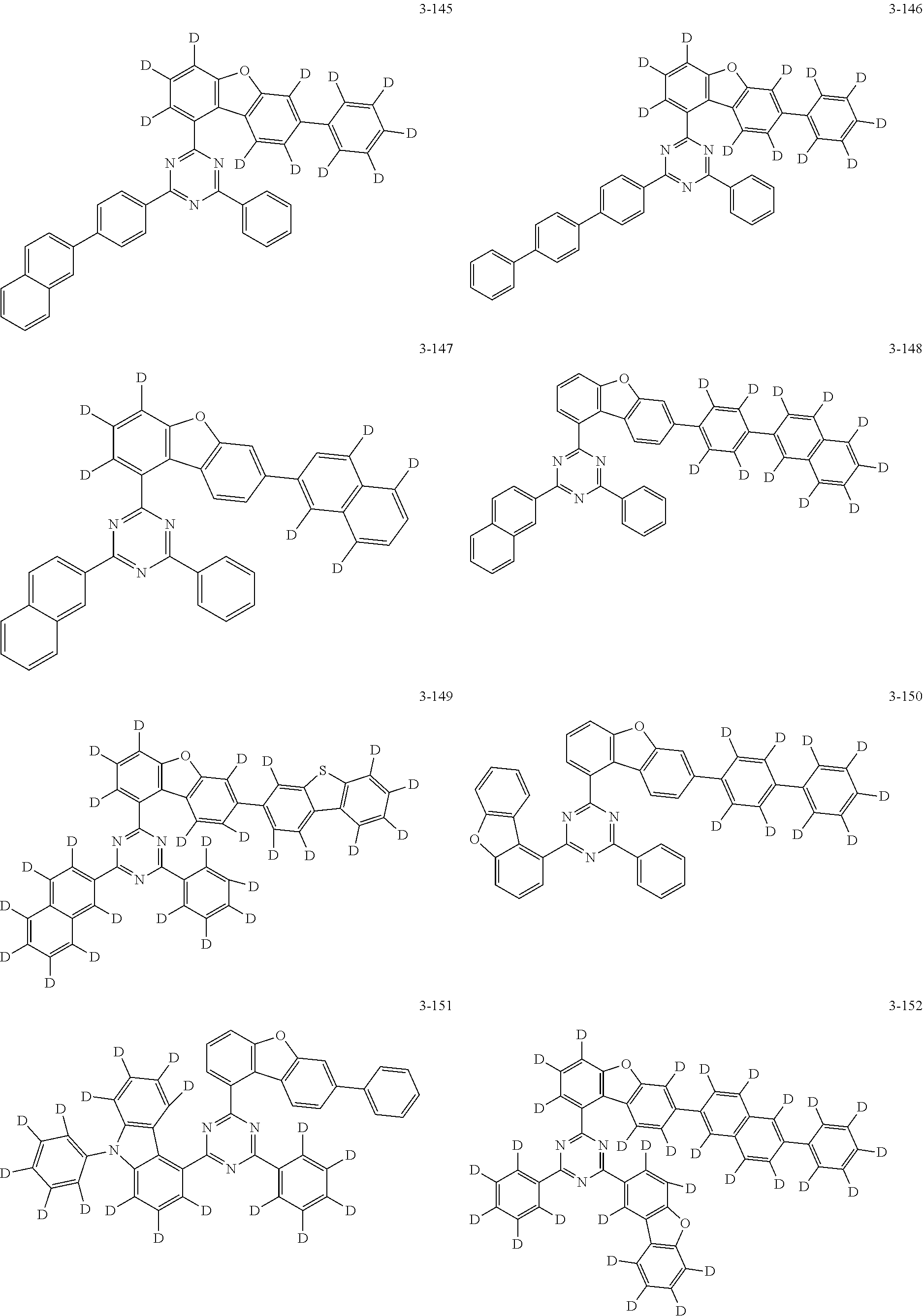

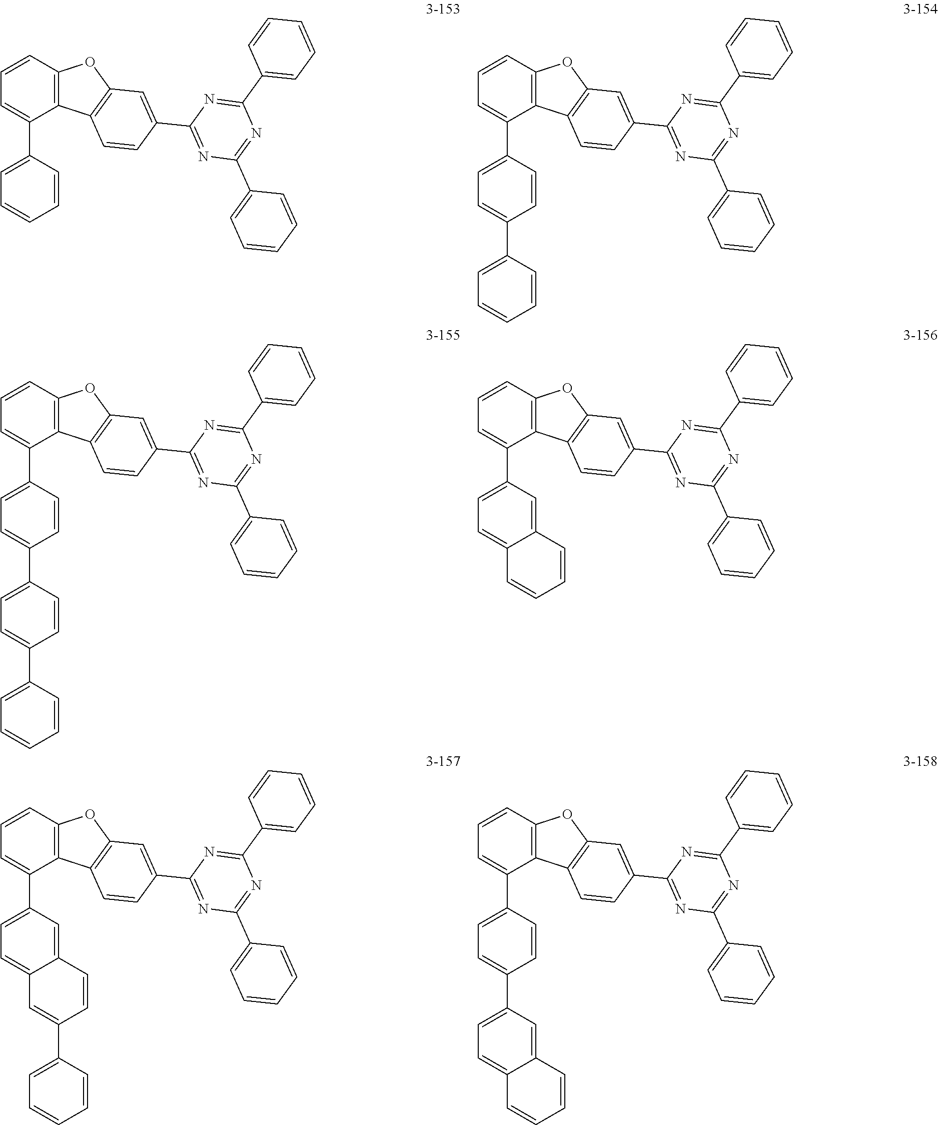 [SMILES: c1ccc(-c2nc(-c3ccccc3)nc(-c3ccc4c(c3)oc3cccc(-c5ccccc5)c34)n2)cc1 ; c1ccc(-c2ccc(-c3cccc4oc5cc(-c6nc(-c7ccccc7)nc(-c7ccccc7)n6)ccc5c34)cc2)cc1 ; c1ccc(-c2ccc(-c3ccc(-c4cccc5oc6cc(-c7nc(-c8ccccc8)nc(-c8ccccc8)n7)ccc6c45)cc3)cc2)cc1 ; c1ccc(-c2nc(-c3ccccc3)nc(-c3ccc4c(c3)oc3cccc(-c5ccc6ccccc6c5)c34)n2)cc1 ; c1ccc(-c2ccc3cc(-c4cccc5oc6cc(-c7nc(-c8ccccc8)nc(-c8ccccc8)n7)ccc6c45)ccc3c2)cc1 ; c1ccc(-c2nc(-c3ccccc3)nc(-c3ccc4c(c3)oc3cccc(-c5ccc(-c6ccc7ccccc7c6)cc5)c34)n2)cc1]

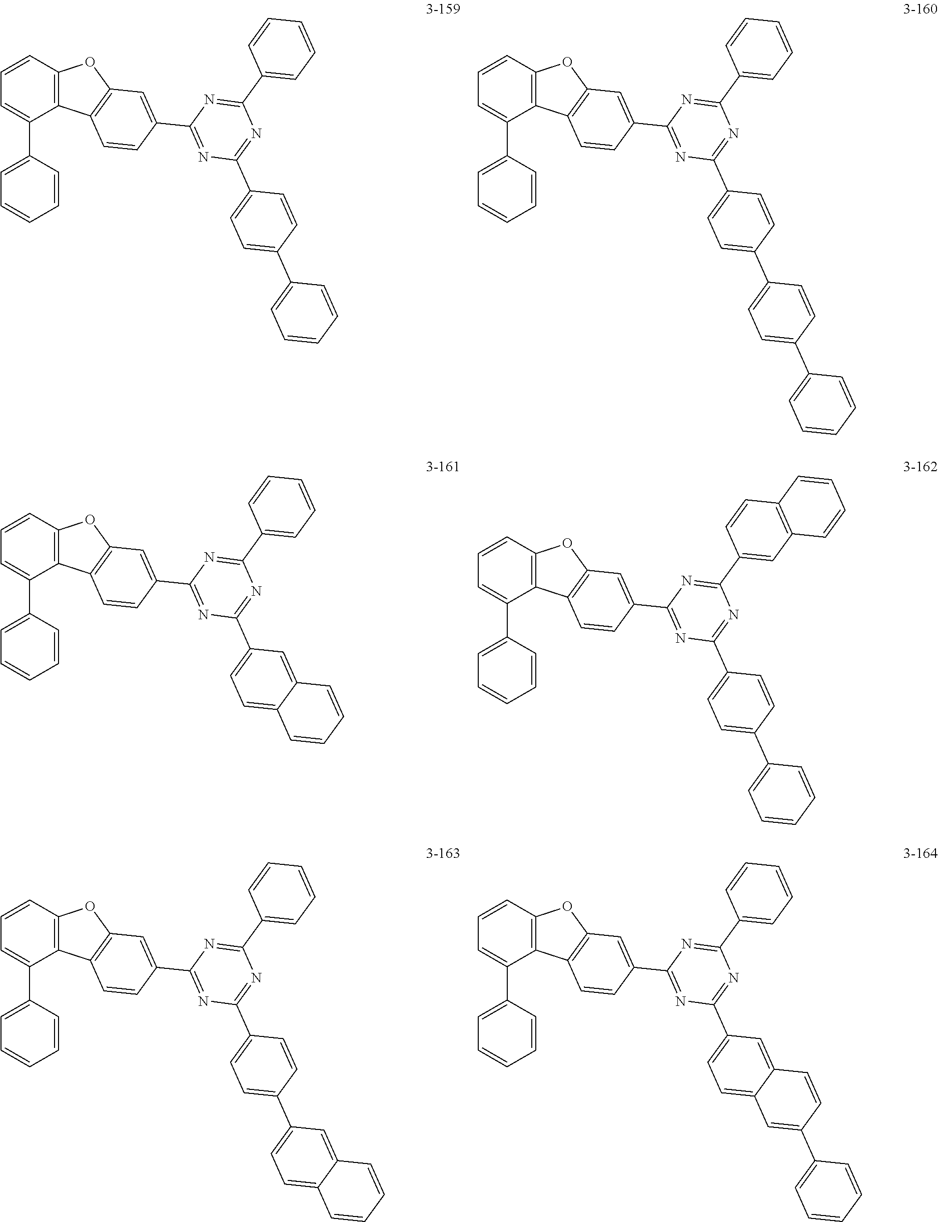

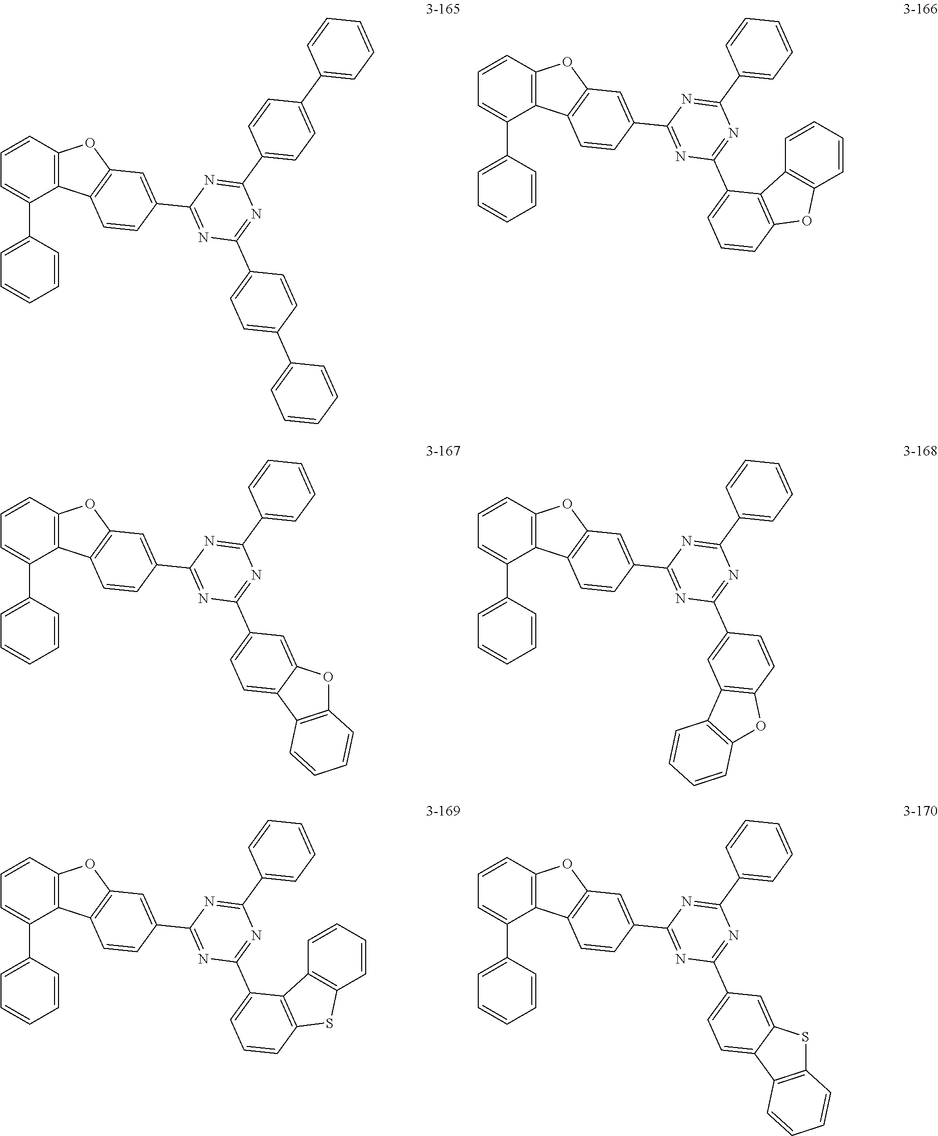

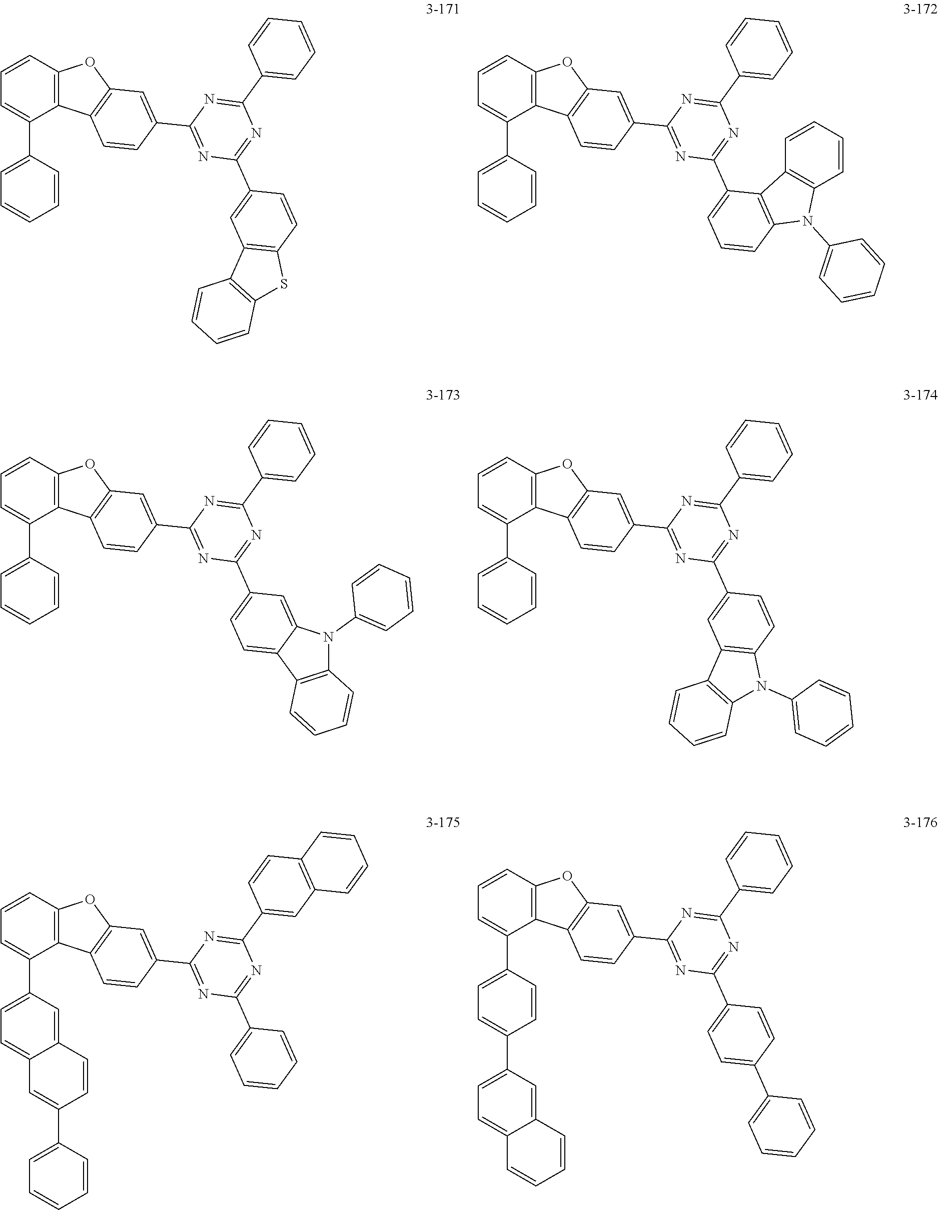

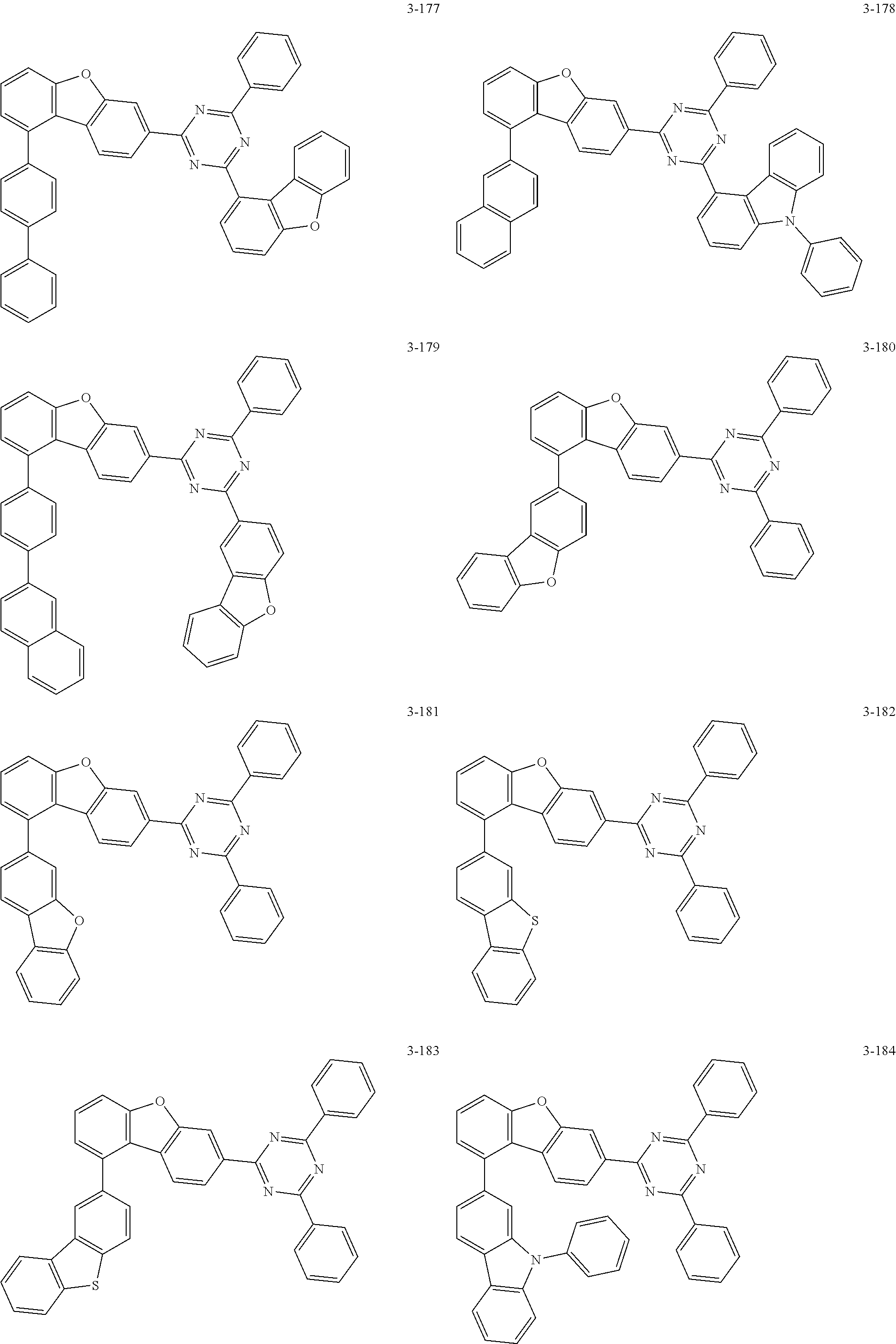

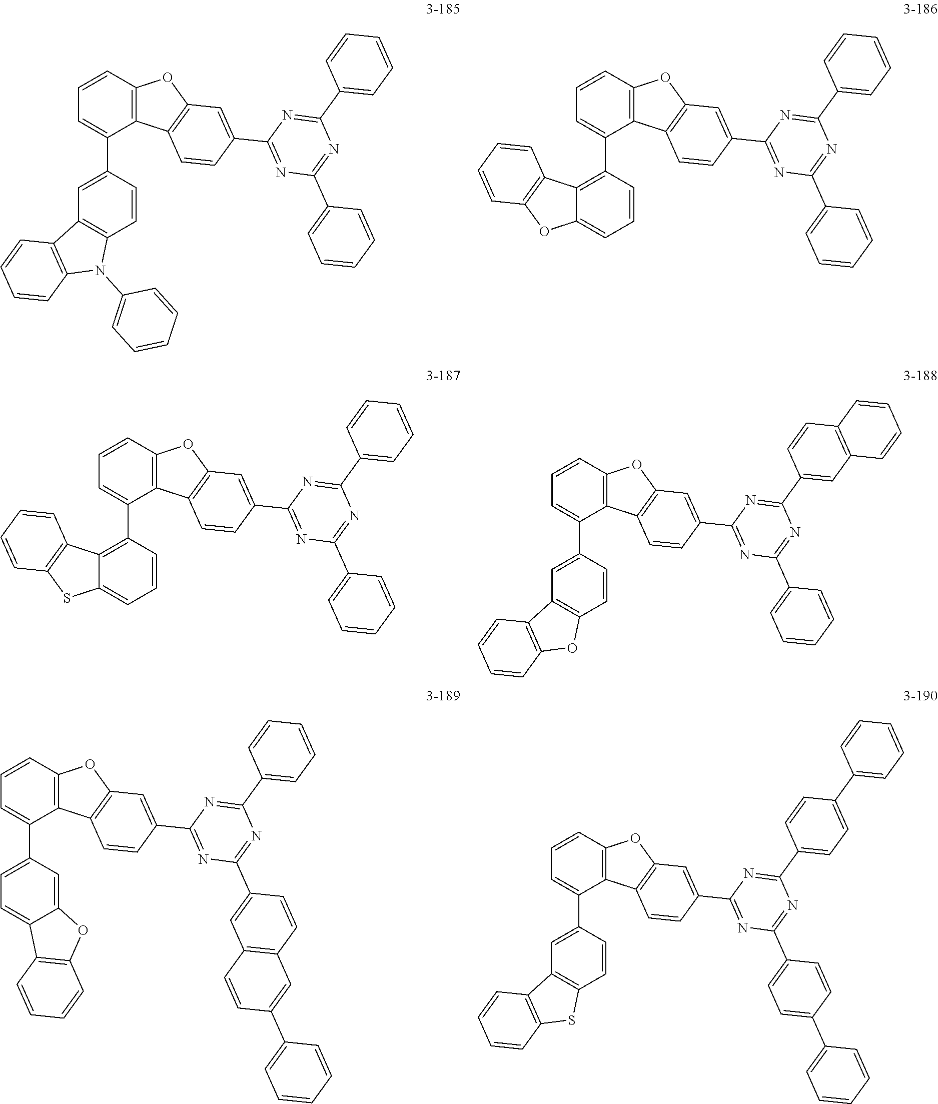

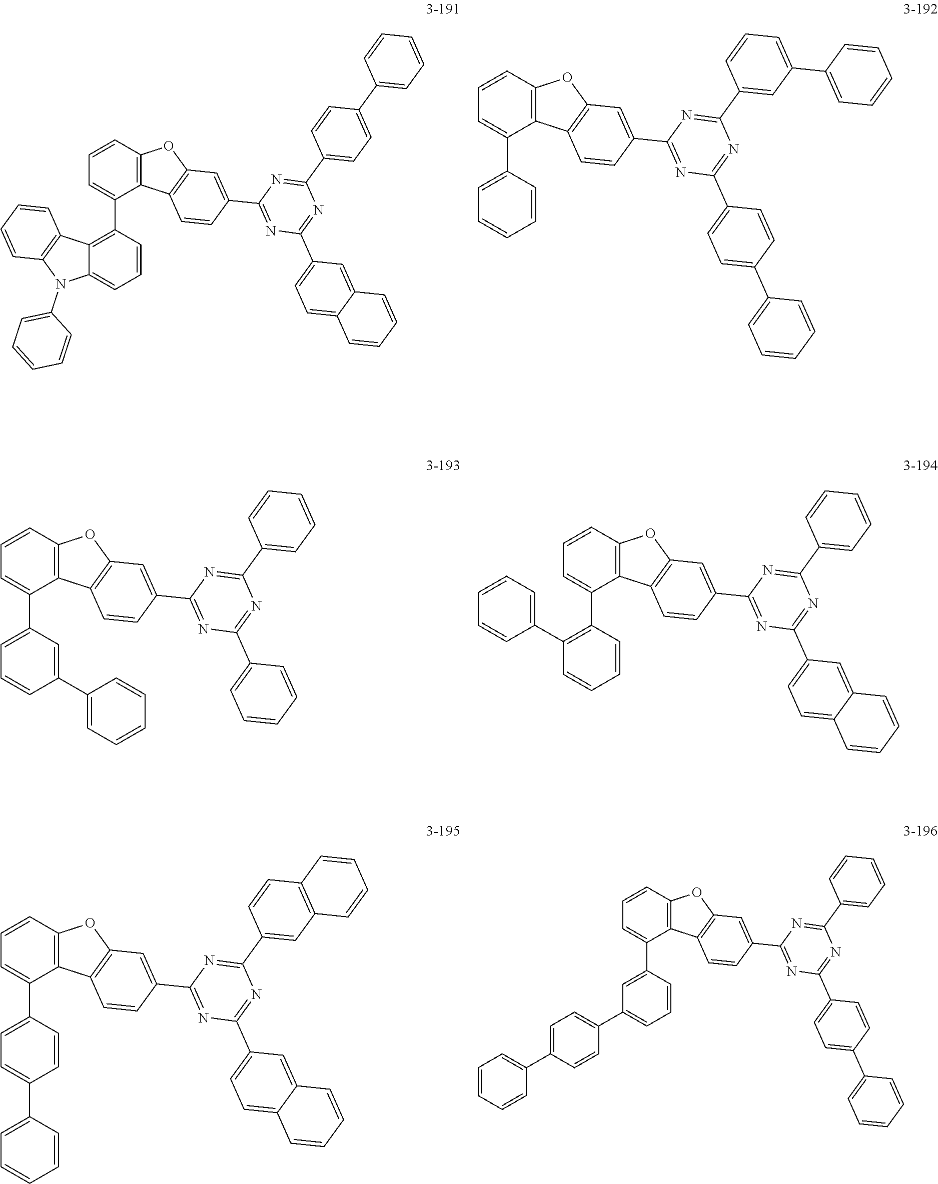

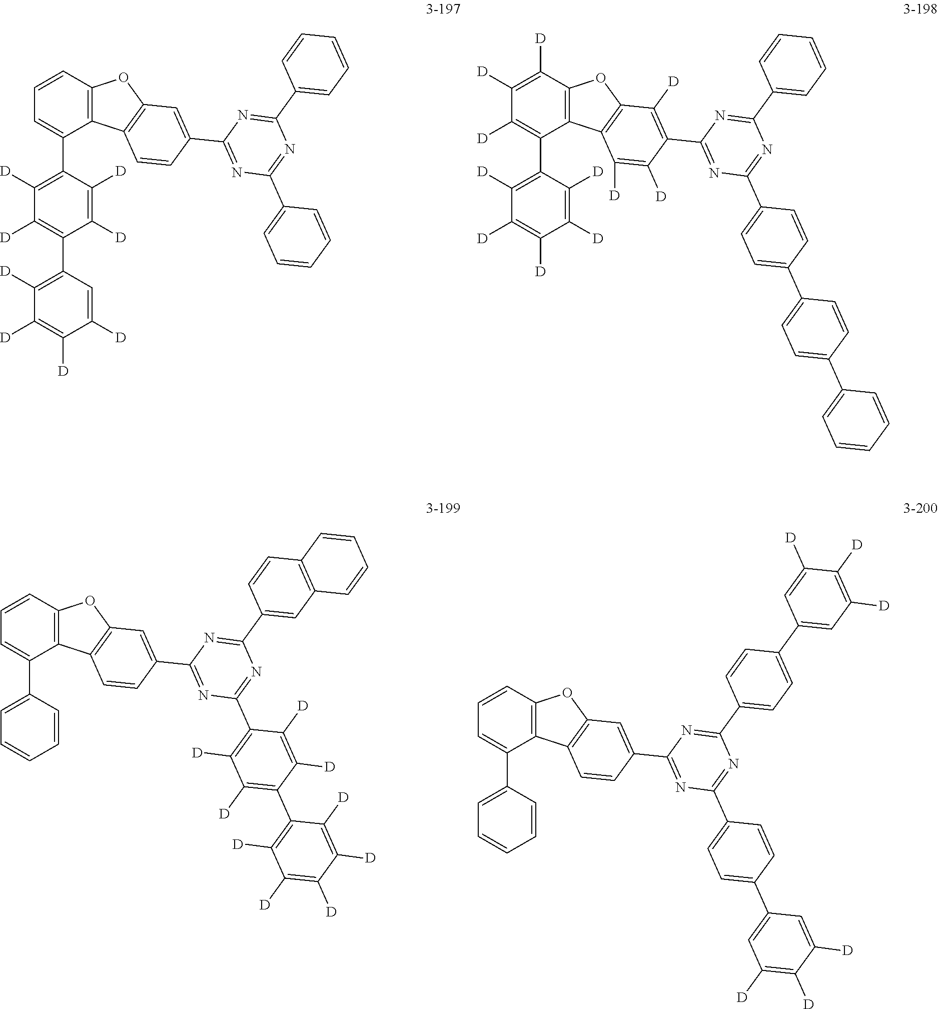

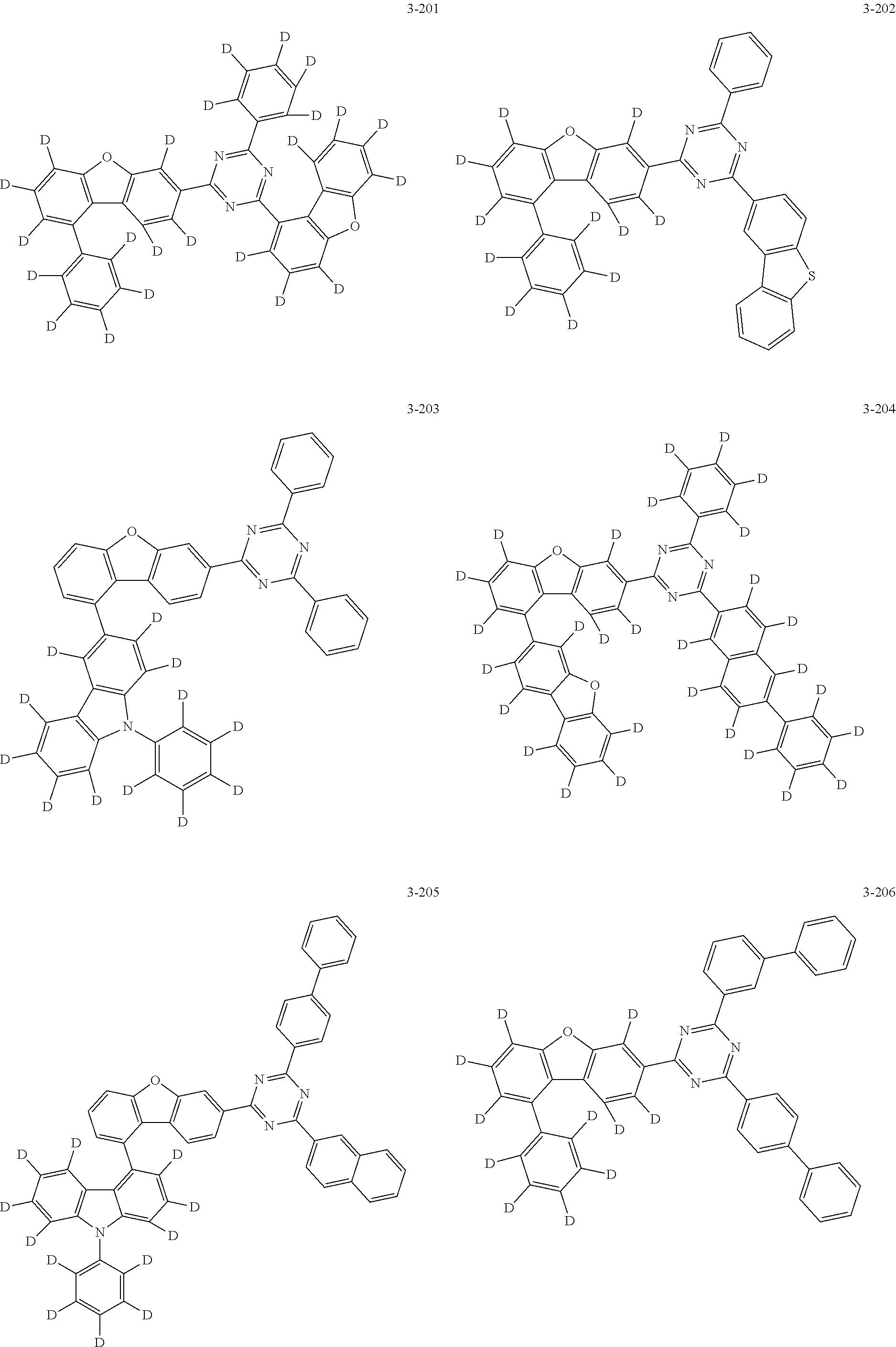

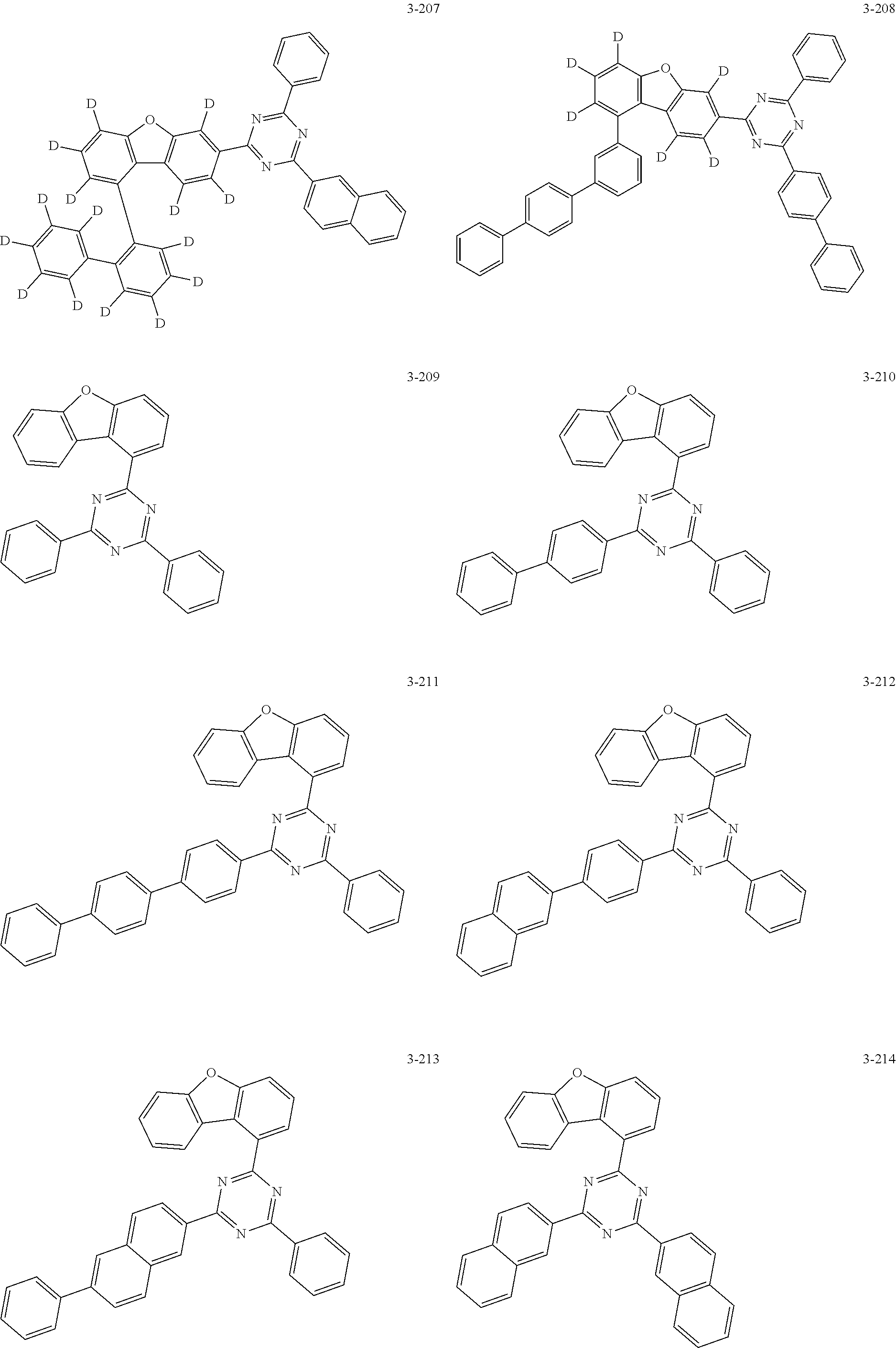

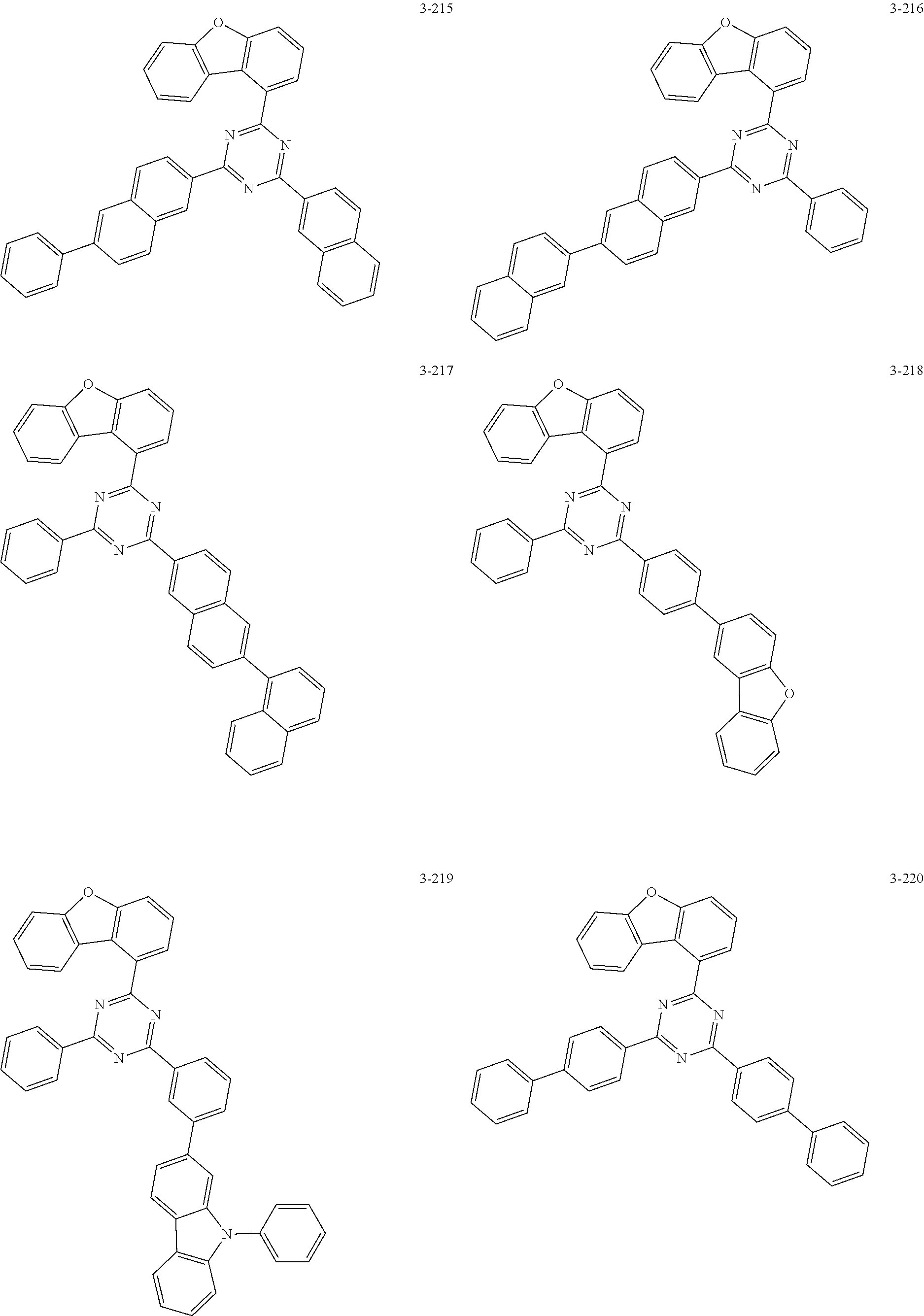 [SMILES: c1ccc(-c2ccc3cc(-c4nc(-c5ccc6ccccc6c5)nc(-c5cccc6oc7ccccc7c56)n4)ccc3c2)cc1 ; c1ccc(-c2nc(-c3ccc4cc(-c5ccc6ccccc6c5)ccc4c3)nc(-c3cccc4oc5ccccc5c34)n2)cc1 ; c1ccc(-c2nc(-c3ccc4cc(-c5cccc6ccccc56)ccc4c3)nc(-c3cccc4oc5ccccc5c34)n2)cc1 ; c1ccc(-c2nc(-c3ccc(-c4ccc5oc6ccccc6c5c4)cc3)nc(-c3cccc4oc5ccccc5c34)n2)cc1 ; c1ccc(-c2nc(-c3cccc(-c4ccc5c6ccccc6n(-c6ccccc6)c5c4)c3)nc(-c3cccc4oc5ccccc5c34)n2)cc1 ; c1ccc(-c2ccc(-c3nc(-c4ccc(-c5ccccc5)cc4)nc(-c4cccc5oc6ccccc6c45)n3)cc2)cc1]

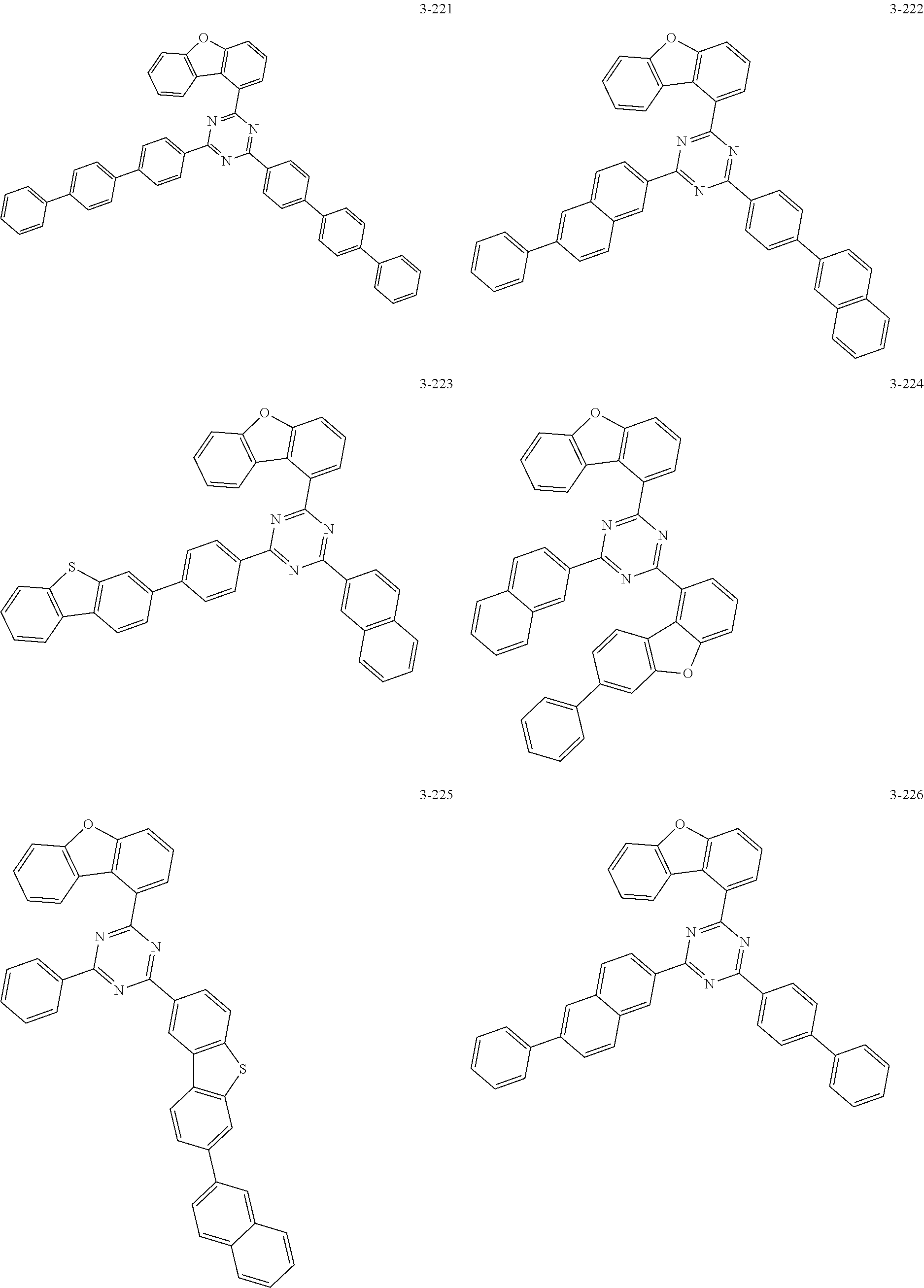

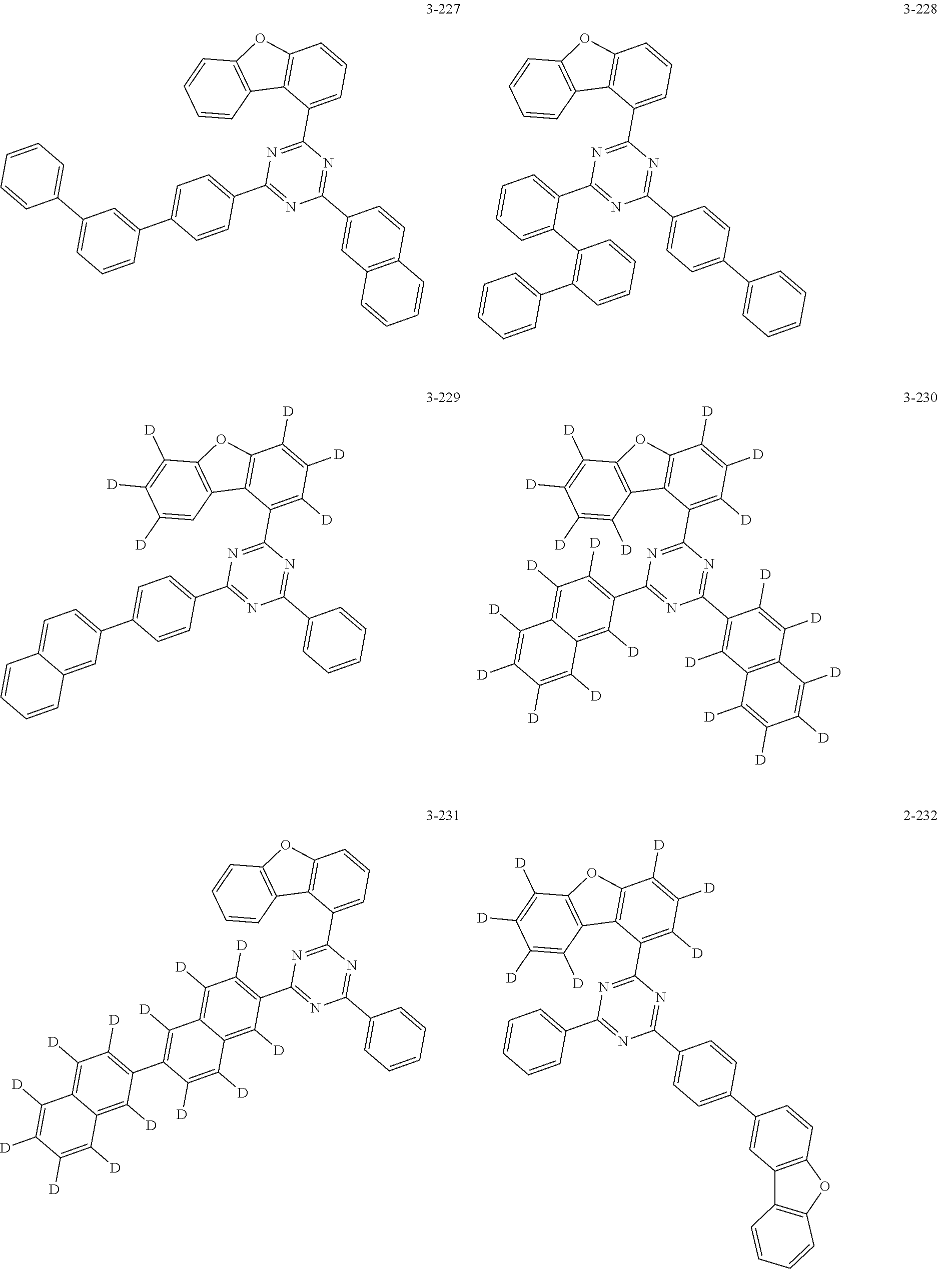

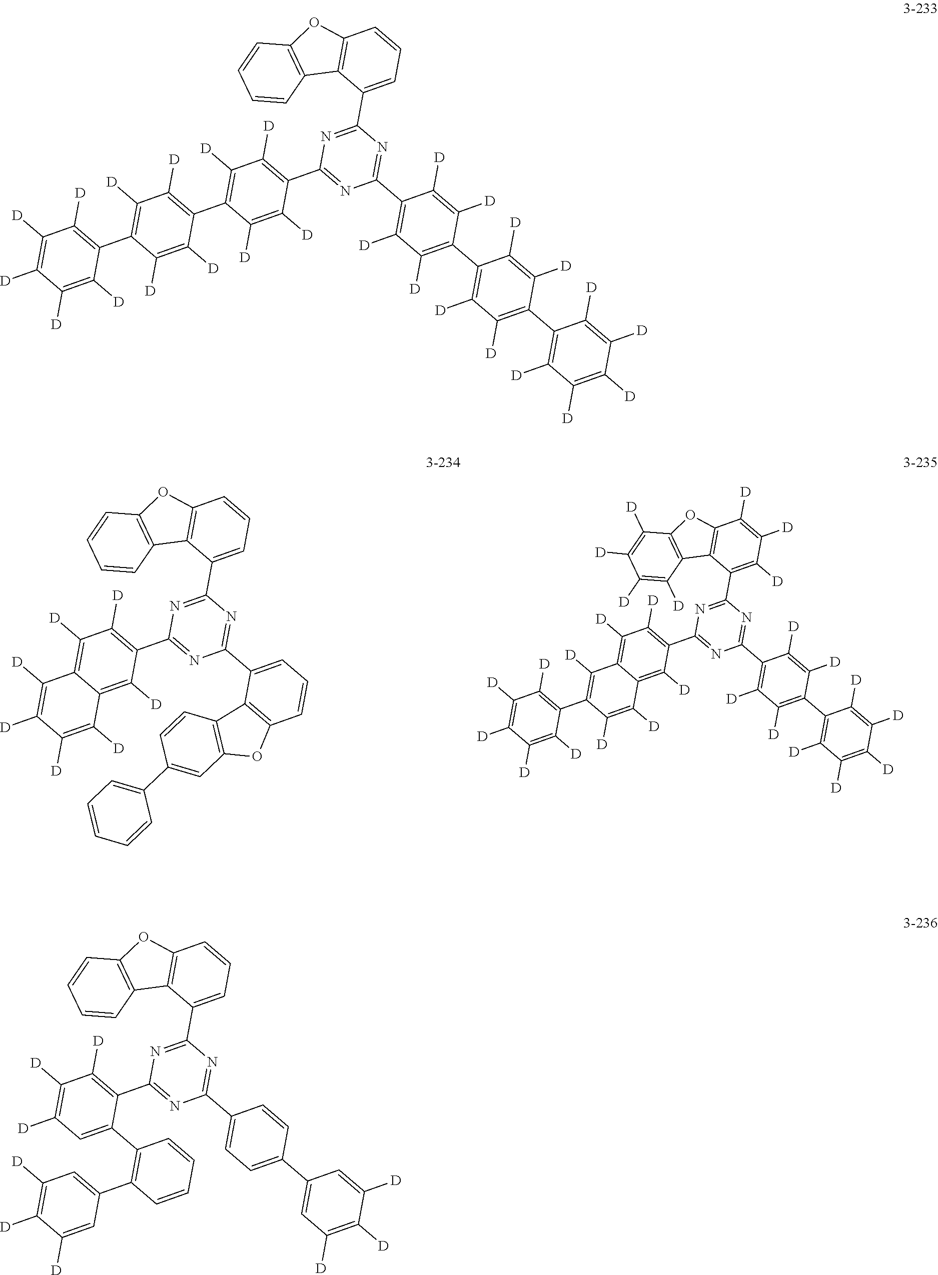

When the heterocyclic compound represented by Chemical Formula 1 and the heterocyclic compound represented by Chemical Formula 2 are included at the same time, effects of more superior efficiency and lifetime are obtained. From this, it may be expected that an exciplex phenomenon occurs when the two compounds are included at the same time.

The exciplex phenomenon is a phenomenon of releasing energy having sizes of a donor (p-host) HOMO energy level and an acceptor (n-host) LUMO energy level due to electron exchanges between two molecules. When the exciplex phenomenon occurs between two molecules, reverse intersystem crossing (RISC) occurs, and as a result, internal quantum efficiency of fluorescence may increase up to 100%. When a donor (p-host) having a favorable hole transport ability and an acceptor (n-host) having a favorable electron transport ability are used as a host of a light emitting layer, holes are injected to the p-host and electrons are injected to the n-host, and thus a driving voltage may be lowered, which resultantly helps with enhancement in the lifetime. In other words, when the compound represented by Chemical Formula 2 is used as the donor and the compound represented by Chemical Formula 1 is used as the acceptor, excellent device properties are obtained.

In one embodiment of the present disclosure, when the heterocyclic compound represented by Chemical Formula 1 and the heterocyclic compound represented by Chemical Formula 2 are included at the same time, at least one of the compounds may not include deuterium as a substituent, or may have a deuterium content of greater than 0%, 1% or greater, 10% or greater, 20% or greater, 30% or greater, 40% or greater or 50% or greater, and 100% or less, 90% or less, 80% or less, 70% or less or 60% or less with respect to the total number of hydrogen atoms and deuterium atoms.

In another embodiment of the present disclosure, at least one of the compounds may not include deuterium as a substituent, or may have a deuterium content of 1% to 100% with respect to the total number of hydrogen atoms and deuterium atoms.

In another embodiment of the present disclosure, at least one of the compounds may not include deuterium as a substituent, or may have a deuterium content of 20% to 90% with respect to the total number of hydrogen atoms and deuterium atoms.

In another embodiment of the present disclosure, at least one of the compounds may not include deuterium as a substituent, or may have a deuterium content of 30% to 80% with respect to the total number of hydrogen atoms and deuterium atoms.

In another embodiment of the present disclosure, at least one of the compounds may not include deuterium as a substituent, or may have a deuterium content of 50% to 70% with respect to the total number of hydrogen atoms and deuterium atoms.

In addition, one embodiment of the present disclosure provides a composition for an organic material layer, the composition including: the heterocyclic compound represented by Chemical Formula 1; and the heterocyclic compound represented by Chemical Formula 2.

Specific descriptions on the heterocyclic compound represented by Chemical Formula 1 and the heterocyclic compound represented by Chemical Formula 2 are the same as the descriptions provided above.

In one embodiment of the present disclosure, the heterocyclic compound represented by Chemical Formula 1 and the heterocyclic compound represented by Chemical Formula 2 may have a weight ratio of 1:9 to 9:1, 1:9 to 5:5 or 2:8 to 5:5 in the composition for an organic material layer, however, the ratio is not limited thereto.

The composition for an organic material layer may be used when forming an organic material of an organic light emitting device, and particularly, may be more preferably used when forming a host of a light emitting layer.

In one embodiment of the present disclosure, the organic material layer includes the heterocyclic compound represented by Chemical Formula 1 and the heterocyclic compound represented by Chemical Formula 2, and a phosphorescent dopant may be used therewith.

As the phosphorescent dopant material, those known in the art may be used. For example, phosphorescent dopant materials represented by LL′MX′, LL′L″M, LMX′X″, L₂MX′ and L₃M may be used, however, the scope of the present disclosure is not limited by these examples.

M may be iridium, platinum, osmium or the like.

L is an anionic bidentate ligand coordinated to M by sp² carbon and heteroatom, and X may function to trap electrons or holes. Nonlimiting examples of L may include 2-(1-naphthyl)benzoxazole, 2-phenylbenzoxazole, 2-phenylbenzothiazole, 7,8-benzoquinoline, phenylpyridine, benzothiophenylpyridine, 3-methoxy-2-phenylpyridine, thiophenylpyridine, tolylpyridine and the like. Nonlimiting examples of X′ and X″ may include acetylacetonate (acac), hexafluoroacetylacetonate, salicylidene, picolinate, 8-hydroxyquinolinate and the like.

Specific examples of the phosphorescent dopant are shown below, however, the phosphorescent dopant is not limited to these examples.

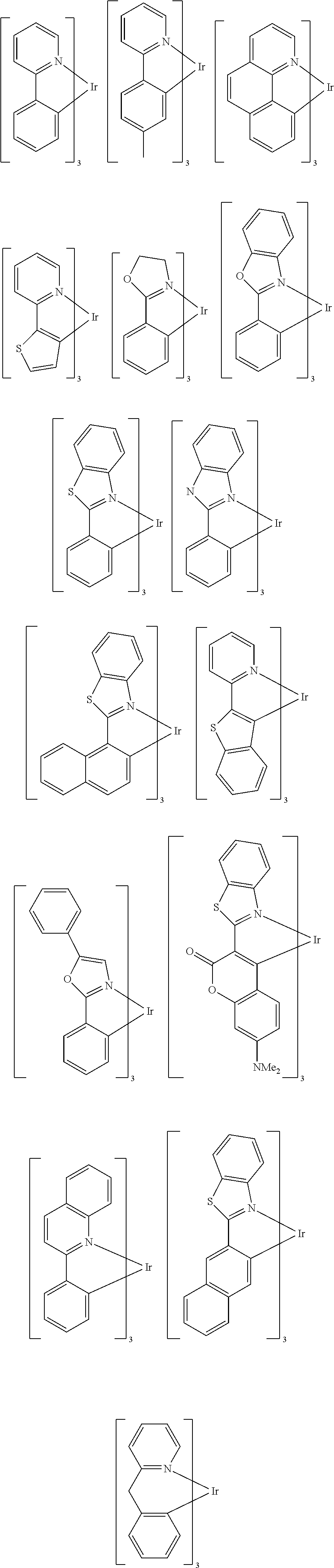

In one embodiment of the present disclosure, the organic material layer includes the heterocyclic compound represented by Chemical Formula 1 and the heterocyclic compound represented by Chemical Formula 2, and an iridium-based dopant may be used therewith.

In one embodiment of the present disclosure, as the iridium-based dopant, (piq)₂(Ir)(acac) may be used as a red phosphorescent dopant, or Ir(ppy)₃ may be used as a green phosphorescent dopant.

In one embodiment of the present disclosure, the content of the dopant may be from 1% to 15%, preferably from 2% to 10% and more preferably from 3% to 7% based on the total weight of the light emitting layer.

In the organic light emitting device according to one embodiment of the present disclosure, the organic material layer includes an electron injection layer or an electron transport layer, and the electron injection layer or the electron transport layer may include the heterocyclic compound represented by Chemical Formula 1.

In the organic light emitting device according to another embodiment of the present disclosure, the organic material layer includes an electron blocking layer or a hole blocking layer, and the electron blocking layer or the hole blocking layer may include the heterocyclic compound represented by Chemical Formula 1.

In the organic light emitting device according to another embodiment, the organic material layer includes an electron transport layer, a light emitting layer or a hole blocking layer, and the electron transport layer, the light emitting layer or the hole blocking layer may include the heterocyclic compound represented by Chemical Formula 1.

In the organic light emitting device according to another embodiment, the organic material layer includes a light emitting layer, and the light emitting layer may include the heterocyclic compound represented by Chemical Formula 1.

In the organic light emitting device according to another embodiment, the organic material layer includes a light emitting layer, and the light emitting layer may include the heterocyclic compound represented by Chemical Formula 1 and the heterocyclic compound represented by Chemical Formula 2.

In the organic light emitting device according to another embodiment, the organic material layer includes a light emitting layer, the light emitting layer includes a host material, and the host material may include the heterocyclic compound represented by Chemical Formula 1.

In the organic light emitting device according to another embodiment, the light emitting layer may include two or more host materials. At least one of the host materials may include the heterocyclic compound represented by Chemical Formula 1, and another one may include the heterocyclic compound represented by Chemical Formula 2.

In the organic light emitting device according to another embodiment, two or more host materials may be pre-mixed and used in the light emitting layer. At least one of the two or more host materials may include the heterocyclic compound represented by Chemical Formula 1, and another one may include the heterocyclic compound represented by Chemical Formula 2.

The pre-mixing means, before depositing the two or more host materials on the organic material layer, putting and mixing the materials first in one source of supply.

The organic light emitting device according to one embodiment of the present disclosure may further include one, or two or more layers selected from the group consisting of a light emitting layer, a hole injection layer, a hole transport layer, an electron injection layer, an electron transport layer, an electron blocking layer and a hole blocking layer.

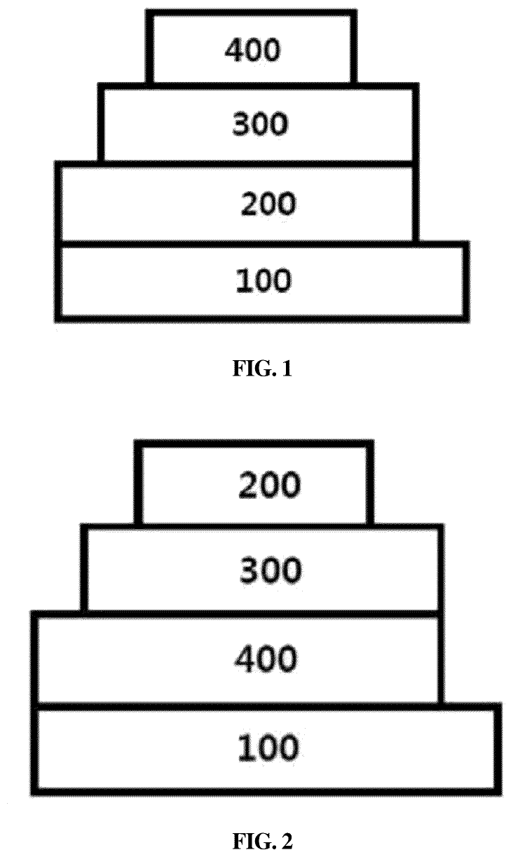

FIGS. 1 to 3 illustrate a lamination order of electrodes and organic material layers of the organic light emitting device according to one embodiment of the present disclosure. However, it is not intended that the scope of the present application be limited by these drawings, and structures of organic light emitting devices known in the art may also be applied to the present application.

FIG. 1 illustrates an organic light emitting device in which a positive electrode 200, an organic material layer 300 and a negative electrode 400 are sequentially laminated on a substrate 100. However, the structure is not limited only to such a structure, and as illustrated in FIG. 2, an organic light emitting device in which a negative electrode, an organic material layer and a positive electrode are sequentially laminated on a substrate may also be obtained.

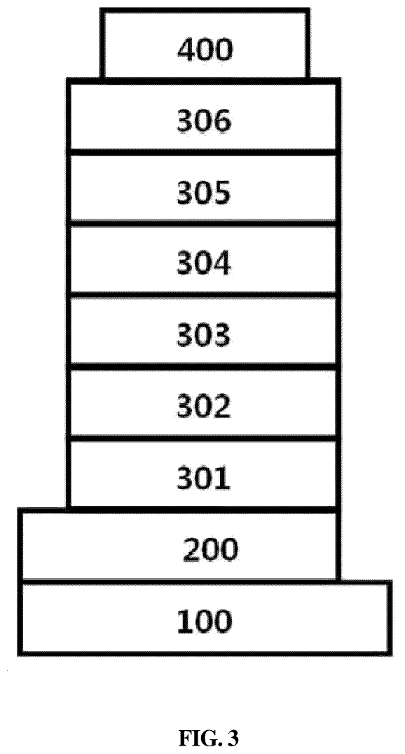

FIG. 3 illustrates a case of the organic material layer being a multilayer. An organic light emitting device according to FIG. 3 includes a hole injection layer 301, a hole transport layer 302, a light emitting layer 303, a hole blocking layer 304, an electron transport layer 305 and an electron injection layer 306. However, the scope of the present application is not limited by such a lamination structure, and as necessary, the layers other than the light emitting layer may not be included, and other necessary functional layers may be further added.

One embodiment of the present disclosure provides a method for manufacturing an organic light emitting device, the method including: preparing a substrate; forming a first electrode on the substrate; forming one or more organic material layers on the first electrode; and forming a second electrode on the one or more organic material layers, wherein the forming of one or more organic material layers includes forming the one or more organic material layers using the composition for an organic material layer according to one embodiment of the present disclosure.

In one embodiment of the present disclosure, the forming of organic material layers may be forming the organic material layers using a thermal vacuum deposition method after pre-mixing the heterocyclic compound represented by Chemical Formula 1 and the heterocyclic compound represented by Chemical Formula 2.

The pre-mixing means, before depositing the heterocyclic compound represented by Chemical Formula 1 and the heterocyclic compound represented by Chemical Formula 2 on the organic material layer, putting and mixing the materials first in one source of supply.

The pre-mixed material may be referred to as the composition for an organic material layer according to one embodiment of the present application.

The organic material layer including the heterocyclic compound represented by Chemical Formula 1 may further include other materials as necessary.

The organic material layer including the heterocyclic compound represented by Chemical Formula 1 and the heterocyclic compound represented by Chemical Formula 2 at the same time may further include other materials as necessary.

In the organic light emitting device according to one embodiment of the present disclosure, materials other than the heterocyclic compound represented by Chemical Formula 1 and the heterocyclic compound represented by Chemical Formula 2 are illustrated below, however, these are for illustrative purposes only and not for limiting the scope of the present application, and these materials may be replaced by materials known in the art.

As the positive electrode material, materials each having a relatively large work function may be used, and transparent conductive oxides, metals, conductive polymers or the like may be used. Specific examples of the positive electrode material include metals such as vanadium, chromium, copper, zinc and gold, or alloys thereof; metal oxides such as zinc oxide, indium oxide, indium tin oxide (ITO) and indium zinc oxide (IZO); combinations of metals and oxides such as ZnO:Al or SnO₂:Sb; conductive polymers such as poly(3-methylthiophene), poly[3,4-(ethylene-1,2-dioxy)thiophene](PEDOT), polypyrrole and polyaniline, and the like, but are not limited thereto.

As the negative electrode material, materials each having a relatively small work function may be used, and metals, metal oxides, conductive polymers or the like may be used. Specific examples of the negative electrode material include metals such as magnesium, calcium, sodium, potassium, titanium, indium, yttrium, lithium, gadolinium, aluminum, silver, tin and lead, or alloys thereof; multilayer structure materials such as LiF/Al or LiO₂/Al, and the like, but are not limited thereto.

As the hole injection layer material, known hole injection layer materials may be used, and for example, phthalocyanine compounds such as copper phthalocyanine disclosed in U.S. Pat. No. 4,356,429, or starburst-type amine derivatives such as tris(4-carbazoyl-9-ylphenyl)amine (TCTA), 4,4′,4″-tris[phenyl(m-tolyl)amino]triphenylamine (m-MTDATA) or 1,3,5-tris[4-(3-methylphenylphenylamino)phenyl]benzene (m-MTDAPB) described in the literature [Advanced Material, 6, p. 677 (1994)], conductive polymers having solubility such as polyaniline/dodecylbenzenesulfonic acid or poly(3,4-ethylenedioxythiophene)/poly(4-styrenesulfonate), polyaniline/camphor sulfonic acid or polyaniline/poly(4-styrenesulfonate), and the like, may be used.

As the hole transport layer material, pyrazoline derivatives, arylamine-based derivatives, stilbene derivatives, triphenyldiamine derivatives and the like may be used, and low molecular or high molecular materials may also be used.

As the electron transport layer material, metal complexes of oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and derivatives thereof, diphenoquinone derivatives, 8-hydroxyquinoline and derivatives thereof, and the like, may be used, and high molecular materials as well as low molecular materials may also be used.

As examples of the electron injection layer material, LiF is typically used in the art, however, the present application is not limited thereto.

As the light emitting layer material, red, green or blue light emitting materials may be used, and as necessary, two or more light emitting materials may be mixed and used. Herein, the two or more light emitting materials may be deposited as individual sources of supply or pre-mixed and deposited as one source of supply when used. In addition, fluorescent materials may also be used as the light emitting layer material, however, phosphorescent materials may also be used. As the light emitting layer material, materials emitting light alone by binding holes and electrons injected from a positive electrode and a negative electrode, respectively, may be used, however, materials having a host material and a dopant material involved in light emission together may also be used.

When hosts of the light emitting layer material are mixed and used, same series hosts may be mixed and used, or different series hosts may be mixed and used. For example, any two or more types of materials among n-type host materials and p-type host materials may be selected and used as a host material of a light emitting layer.

The organic light emitting device according to one embodiment of the present disclosure may be a top-emission type, a bottom-emission type or a dual-emission type depending on the materials used.

The heterocyclic compound according to one embodiment of the present disclosure may also be used in an organic electronic device including an organic solar cell, an organic photo conductor, an organic transistor and the like under a principle similar to that in the organic light emitting device.

Hereinafter, preferred examples are provided to help to understand the present disclosure, however, the following examples are only provided to more readily understand the present disclosure, and the present disclosure is not limited thereto.

### Preparation Example 1. Preparation of Compound 1-1

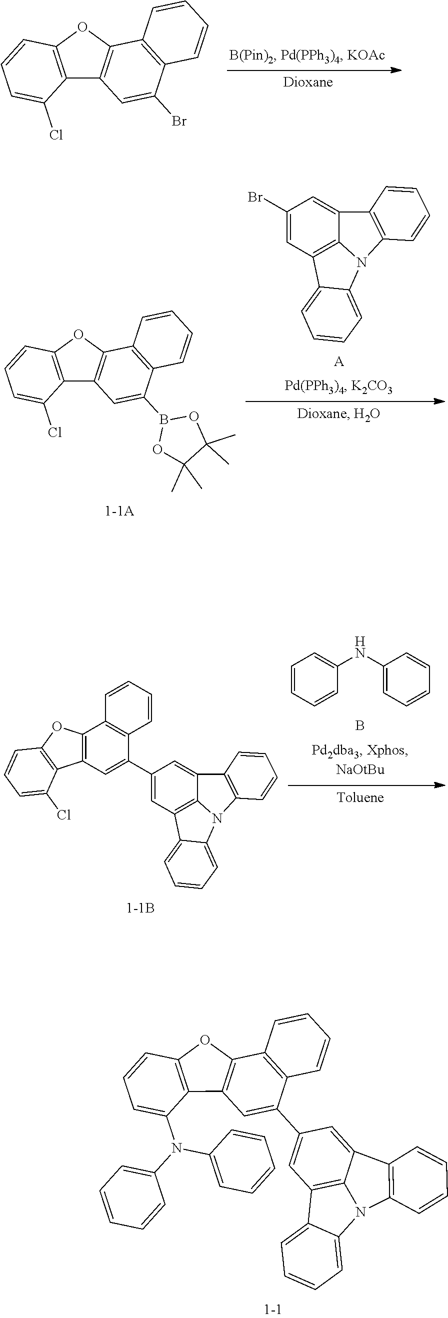 [SMILES: Clc1cccc2oc3c4ccccc4c(Br)cc3c12 ; CC1(C)OB(c2cc3c(oc4cccc(Cl)c43)c3ccccc23)OC1(C)C ; Brc1cc2c3ccccc3n3c4ccccc4c(c1)c23 ; Clc1cccc2oc3c4ccccc4c(-c4cc5c6ccccc6n6c7ccccc7c(c4)c56)cc3c12 ; c1ccc(Nc2ccccc2)cc1 ; c1ccc(N(c2ccccc2)c2cccc3oc4c5ccccc5c(-c5cc6c7ccccc7n7c8ccccc8c(c5)c67)cc4c23)cc1]

### Preparation Example 1-1. Preparation of Compound 1-1A

5-bromo-7-chloronaphtho[1,2-b]benzofuran (10.0 g, 30.3 mmol), bis(pinacolato)diboron (B(Pin)₂) (11.6 g, 45.5 mmol), Pd(PPh₃)₄ (1.8 g, 1.5 mmol) and KOAc (5.9 g, 60.6 mmol) were dissolved in 1,4-dioxane (100 mL), and then the mixture was refluxed for 3 hours.

After the reaction was completed, the reaction material was filtered under reduced pressure at room temperature, and then the solvent of the filtrate was removed using a rotary evaporator. After that, the result was dissolved in dichloromethane (DCM) and purified by silica, and then recrystallized with dichloromethane and methanol to obtain Compound 1-1A (10.3 g, yield 90%).

### Preparation Example 1-2. Preparation of Compound 1-1B

Compound 1-1A (10.3 g, 27.3 mmol), 2-bromoindolo[3,2,1-jk]carbazole (10.5 g, 32.7 mmol), Pd(PPh₃)₄ (1.6 g, 1.4 mmol) and K₂CO₃ (7.5 g, 54.6 mmol) were dissolved in 1,4-dioxane (100 mL) and distilled water (20 mL), and then the mixture was refluxed for 3 hours.

After the reaction was completed, the reaction material was filtered under reduced pressure at room temperature. After that, the result was dissolved in 1,2-dichlorobenzene (DCB) and purified by silica, and then recrystallized with dichloromethane and methanol to obtain Compound 1-1B (11.1 g, yield 81%).

### Preparation Example 1-3. Preparation of Compound 1-1

Compound 1-1B (11.1 g, 22.7 mmol), diphenylamine (3.8 g, 22.7 mmol), Pd₂(dba)₃ (1.0 g, 1.1 mmol), Xphos (1.1 g, 2.3 mmol) and NaOtBu (4.4 g, 45.4 mmol) were dissolved in toluene (110 mL), and then the mixture was refluxed for 3 hours.

After the reaction was completed, the reaction material was filtered under reduced pressure at room temperature. After that, the result was dissolved in 1,2-dichlorobenzene (DCB) and purified by silica, and then recrystallized with 1,2-dichlorobenzene to obtain target Compound 1-1 (11.3 g, yield 80%).

Target compounds were prepared as in the following Table 1 in the same manner as in Preparation Example 1, except that Compound A of the following Table 1 was used instead of 2-bromoindolo[3,2,1-jk]carbazole, and Compound B of the following Table 1 was used instead of diphenylamine.

TABLE 1

| Com- | | | | |
|---|---|---|---|---|
| pound No. | Compound A | Compound B | Target Compound | Yield |
| 1-2 | 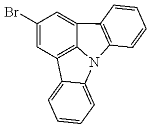 [SMILES: Brc1cc2c3ccccc3n3c4ccccc4c(c1)c23] | 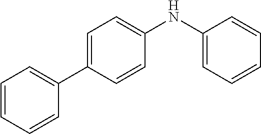 [SMILES: c1ccc(Nc2ccc(-c3ccccc3)cc2)cc1] | 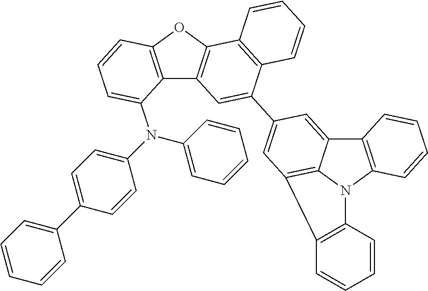 [SMILES: c1ccc(-c2ccc(N(c3ccccc3)c3cccc4oc5c6ccccc6c(-c6cc7c8ccccc8n8c9ccccc9c(c6)c78)cc5c34)cc2)cc1] | 85% |
| 1-3 | 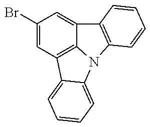 [SMILES: Brc1cc2c3ccccc3n3c4ccccc4c(c1)c23] | 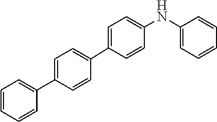 [SMILES: c1ccc(Nc2ccc(-c3ccc(-c4ccccc4)cc3)cc2)cc1] | 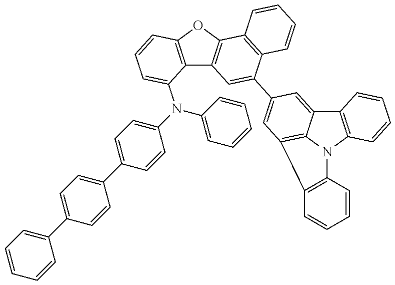 [SMILES: c1ccc(-c2ccc(-c3ccc(N(c4ccccc4)c4cccc5oc6c7ccccc7c(-c7cc8c9ccccc9n9c%10ccccc%10c(c7)c89)cc6c45)cc3)cc2)cc1] | 86% |
| 1-4 | 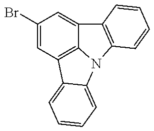 [SMILES: Brc1cc2c3ccccc3n3c4ccccc4c(c1)c23] | 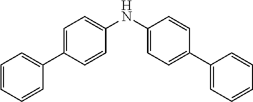 [SMILES: c1ccc(-c2ccc(Nc3ccc(-c4ccccc4)cc3)cc2)cc1] | 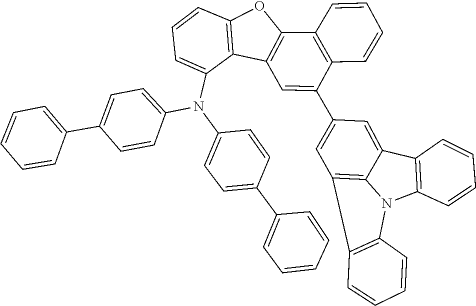 [SMILES: c1ccc(-c2ccc(N(c3ccc(-c4ccccc4)cc3)c3cccc4oc5c6ccccc6c(-c6cc7c8ccccc8n8c9ccccc9c(c6)c78)cc5c34)cc2)cc1] | 77% |
| 1-5 | 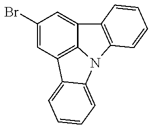 [SMILES: Brc1cc2c3ccccc3n3c4ccccc4c(c1)c23] | 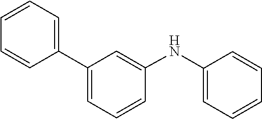 [SMILES: c1ccc(Nc2cccc(-c3ccccc3)c2)cc1] | 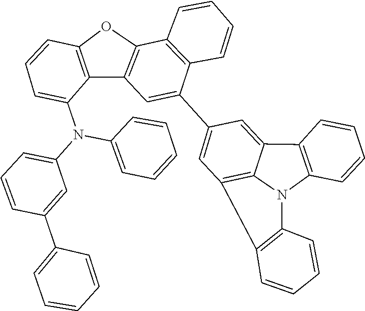 [SMILES: c1ccc(-c2cccc(N(c3ccccc3)c3cccc4oc5c6ccccc6c(-c6cc7c8ccccc8n8c9ccccc9c(c6)c78)cc5c34)c2)cc1] | 70% |
| 1-6 | 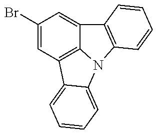 [SMILES: Brc1cc2c3ccccc3n3c4ccccc4c(c1)c23] | 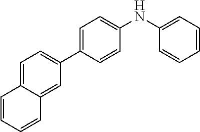 [SMILES: c1ccc(Nc2ccc(-c3ccc4ccccc4c3)cc2)cc1] | 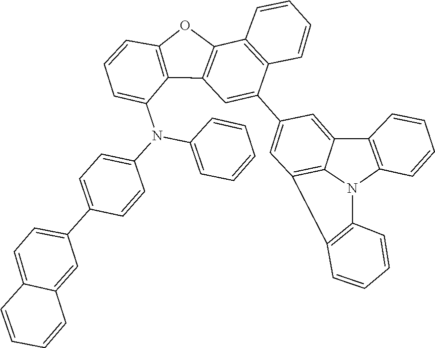 [SMILES: c1ccc(N(c2ccc(-c3ccc4ccccc4c3)cc2)c2cccc3oc4c5ccccc5c(-c5cc6c7ccccc7n7c8ccccc8c(c5)c67)cc4c23)cc1] | 71% |
| 1-7 | 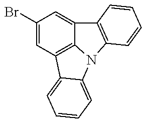 [SMILES: Brc1cc2c3ccccc3n3c4ccccc4c(c1)c23] | 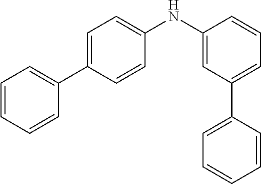 [SMILES: c1ccc(-c2ccc(Nc3cccc(-c4ccccc4)c3)cc2)cc1] | 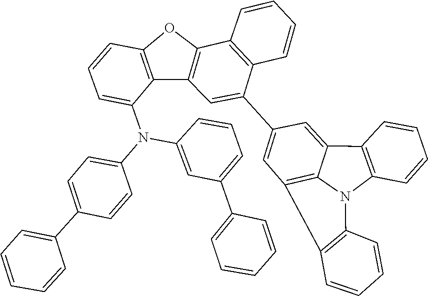 [SMILES: c1ccc(-c2ccc(N(c3cccc(-c4ccccc4)c3)c3cccc4oc5c6ccccc6c(-c6cc7c8ccccc8n8c9ccccc9c(c6)c78)cc5c34)cc2)cc1] | 88% |
| 1-8 | 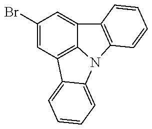 [SMILES: Brc1cc2c3ccccc3n3c4ccccc4c(c1)c23] | 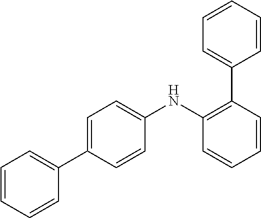 [SMILES: c1ccc(-c2ccc(Nc3ccccc3-c3ccccc3)cc2)cc1] | 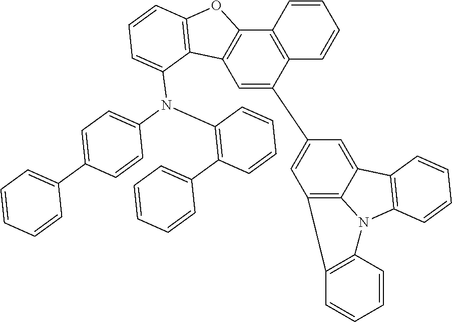 [SMILES: c1ccc(-c2ccc(N(c3ccccc3-c3ccccc3)c3cccc4oc5c6ccccc6c(-c6cc7c8ccccc8n8c9ccccc9c(c6)c78)cc5c34)cc2)cc1] | 76% |
| 1-9 | 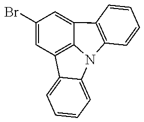 [SMILES: Brc1cc2c3ccccc3n3c4ccccc4c(c1)c23] | 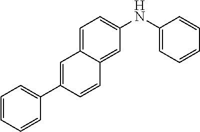 [SMILES: c1ccc(Nc2ccc3cc(-c4ccccc4)ccc3c2)cc1] | 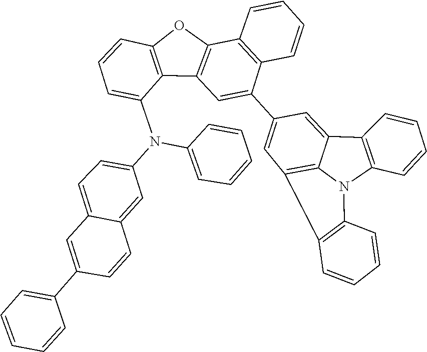 [SMILES: c1ccc(-c2ccc3cc(N(c4ccccc4)c4cccc5oc6c7ccccc7c(-c7cc8c9ccccc9n9c%10ccccc%10c(c7)c89)cc6c45)ccc3c2)cc1] | 71% |
| 1-10 | 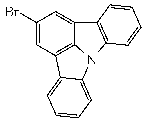 [SMILES: Brc1cc2c3ccccc3n3c4ccccc4c(c1)c23] | 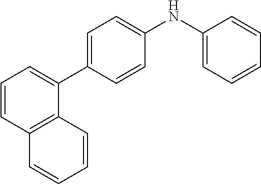 [SMILES: c1ccc(Nc2ccc(-c3cccc4ccccc34)cc2)cc1] | 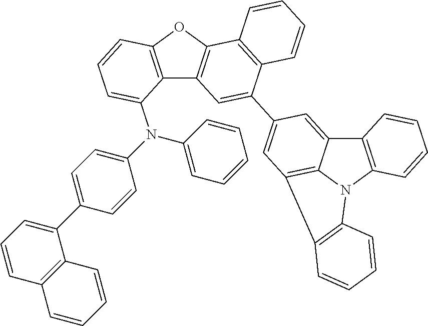 [SMILES: c1ccc(N(c2ccc(-c3cccc4ccccc34)cc2)c2cccc3oc4c5ccccc5c(-c5cc6c7ccccc7n7c8ccccc8c(c5)c67)cc4c23)cc1] | 86% |
| 1-11 | 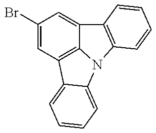 [SMILES: Brc1cc2c3ccccc3n3c4ccccc4c(c1)c23] | 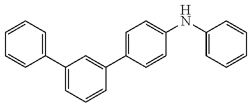 [SMILES: c1ccc(Nc2ccc(-c3cccc(-c4ccccc4)c3)cc2)cc1] | 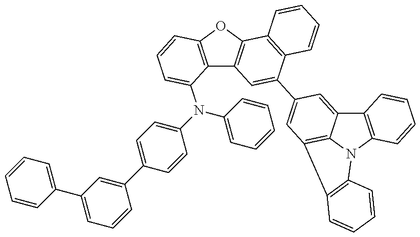 [SMILES: c1ccc(-c2cccc(-c3ccc(N(c4ccccc4)c4cccc5oc6c7ccccc7c(-c7cc8c9ccccc9n9c%10ccccc%10c(c7)c89)cc6c45)cc3)c2)cc1] | 86% |
| 1-12 | 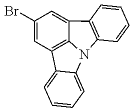 [SMILES: Brc1cc2c3ccccc3n3c4ccccc4c(c1)c23] | 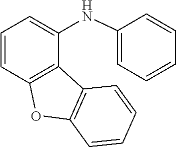 [SMILES: c1ccc(Nc2cccc3oc4ccccc4c23)cc1] | 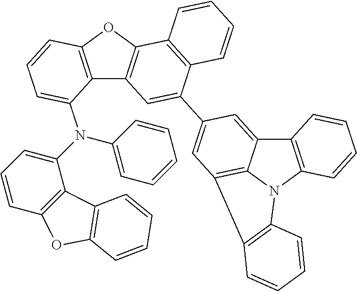 [SMILES: c1ccc(N(c2cccc3oc4ccccc4c23)c2cccc3oc4c5ccccc5c(-c5cc6c7ccccc7n7c8ccccc8c(c5)c67)cc4c23)cc1] | 76% |
| 1-13 | 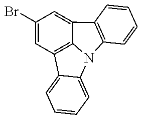 [SMILES: Brc1cc2c3ccccc3n3c4ccccc4c(c1)c23] | 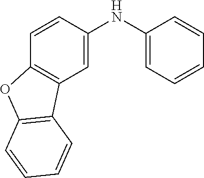 [SMILES: c1ccc(Nc2ccc3oc4ccccc4c3c2)cc1] | 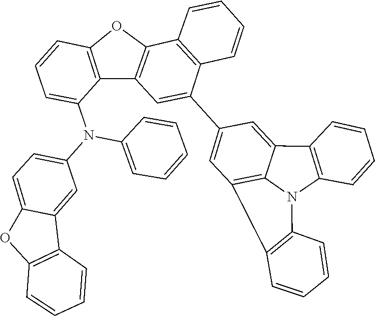 [SMILES: c1ccc(N(c2ccc3oc4ccccc4c3c2)c2cccc3oc4c5ccccc5c(-c5cc6c7ccccc7n7c8ccccc8c(c5)c67)cc4c23)cc1] | 78% |
| 1-14 | 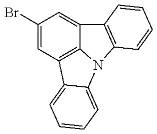 [SMILES: Brc1cc2c3ccccc3n3c4ccccc4c(c1)c23] | 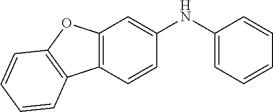 [SMILES: c1ccc(Nc2ccc3c(c2)oc2ccccc23)cc1] | 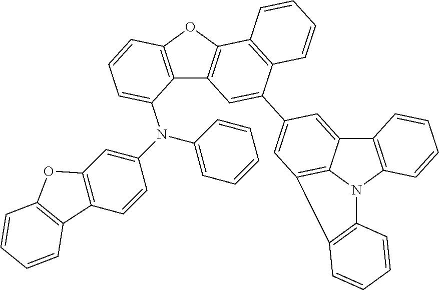 [SMILES: c1ccc(N(c2ccc3c(c2)oc2ccccc23)c2cccc3oc4c5ccccc5c(-c5cc6c7ccccc7n7c8ccccc8c(c5)c67)cc4c23)cc1] | 87% |
| 1-15 | 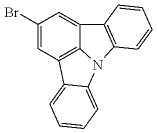 [SMILES: Brc1cc2c3ccccc3n3c4ccccc4c(c1)c23] | 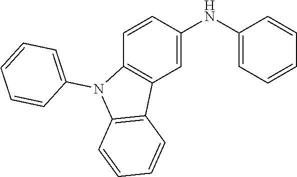 [SMILES: c1ccc(Nc2ccc3c(c2)c2ccccc2n3-c2ccccc2)cc1] | 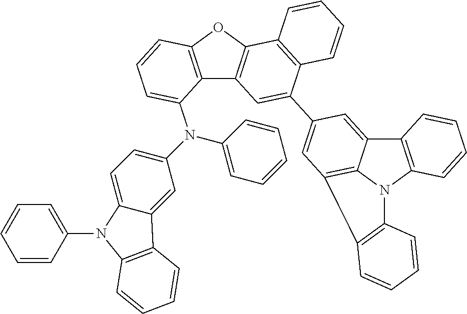 [SMILES: c1ccc(N(c2ccc3c(c2)c2ccccc2n3-c2ccccc2)c2cccc3oc4c5ccccc5c(-c5cc6c7ccccc7n7c8ccccc8c(c5)c67)cc4c23)cc1] | 73% |
| 1-16 | 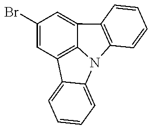 [SMILES: Brc1cc2c3ccccc3n3c4ccccc4c(c1)c23] | 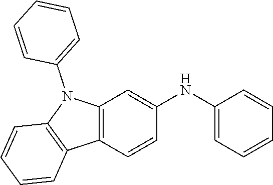 [SMILES: c1ccc(Nc2ccc3c4ccccc4n(-c4ccccc4)c3c2)cc1] | 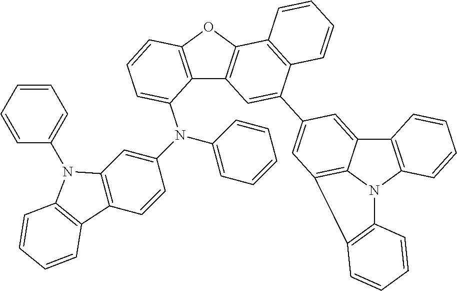 [SMILES: c1ccc(N(c2ccc3c4ccccc4n(-c4ccccc4)c3c2)c2cccc3oc4c5ccccc5c(-c5cc6c7ccccc7n7c8ccccc8c(c5)c67)cc4c23)cc1] | 83% |
| 1-17 | 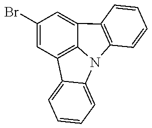 [SMILES: Brc1cc2c3ccccc3n3c4ccccc4c(c1)c23] | 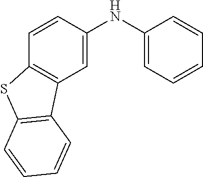 [SMILES: c1ccc(Nc2ccc3sc4ccccc4c3c2)cc1] | 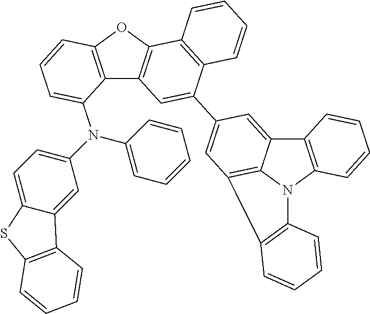 [SMILES: c1ccc(N(c2ccc3sc4ccccc4c3c2)c2cccc3oc4c5ccccc5c(-c5cc6c7ccccc7n7c8ccccc8c(c5)c67)cc4c23)cc1] | 70% |
| 1-18 | 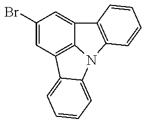 [SMILES: Brc1cc2c3ccccc3n3c4ccccc4c(c1)c23] | 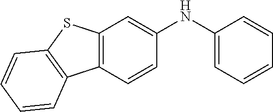 [SMILES: c1ccc(Nc2ccc3c(c2)sc2ccccc23)cc1] | 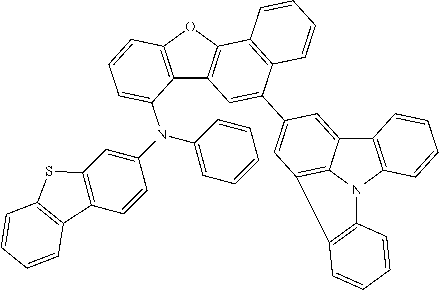 [SMILES: c1ccc(N(c2ccc3c(c2)sc2ccccc23)c2cccc3oc4c5ccccc5c(-c5cc6c7ccccc7n7c8ccccc8c(c5)c67)cc4c23)cc1] | 76% |
| 1-25 | 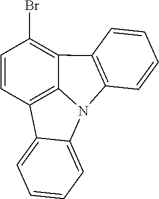 [SMILES: Brc1ccc2c3ccccc3n3c4ccccc4c1c23] | 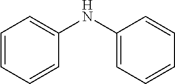 [SMILES: c1ccc(Nc2ccccc2)cc1] | 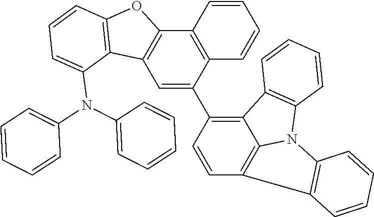 [SMILES: c1ccc(N(c2ccccc2)c2cccc3oc4c5ccccc5c(-c5ccc6c7ccccc7n7c8ccccc8c5c67)cc4c23)cc1] | 77% |
| 1-28 | 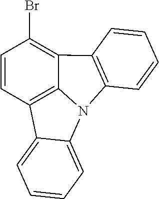 [SMILES: Brc1ccc2c3ccccc3n3c4ccccc4c1c23] | 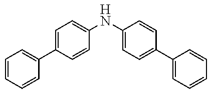 [SMILES: c1ccc(-c2ccc(Nc3ccc(-c4ccccc4)cc3)cc2)cc1] | 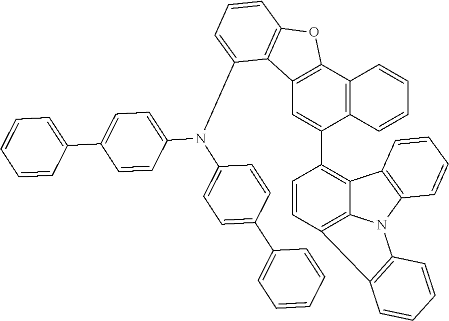 [SMILES: c1ccc(-c2ccc(N(c3ccc(-c4ccccc4)cc3)c3cccc4oc5c6ccccc6c(-c6ccc7c8ccccc8n8c9ccccc9c6c78)cc5c34)cc2)cc1] | 79% |
| 1-30 | 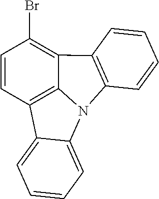 [SMILES: Brc1ccc2c3ccccc3n3c4ccccc4c1c23] | 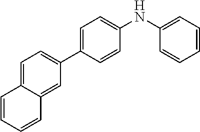 [SMILES: c1ccc(Nc2ccc(-c3ccc4ccccc4c3)cc2)cc1] | 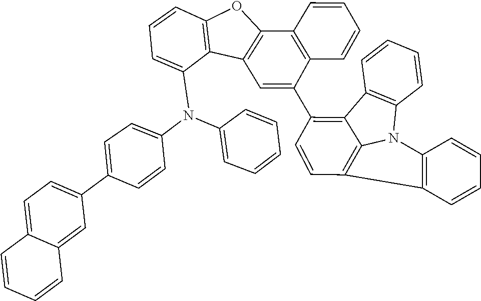 [SMILES: c1ccc(N(c2ccc(-c3ccc4ccccc4c3)cc2)c2cccc3oc4c5ccccc5c(-c5ccc6c7ccccc7n7c8ccccc8c5c67)cc4c23)cc1] | 75% |
| 1-37 | 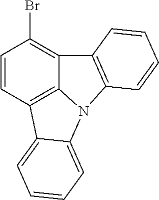 [SMILES: Brc1ccc2c3ccccc3n3c4ccccc4c1c23] | 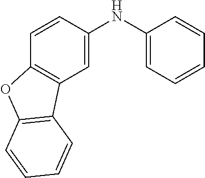 [SMILES: c1ccc(Nc2ccc3oc4ccccc4c3c2)cc1] | 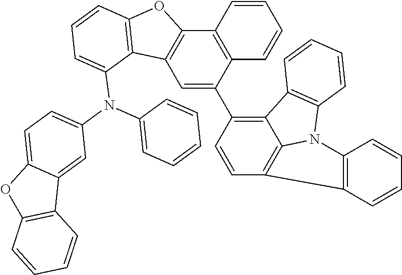 [SMILES: c1ccc(N(c2ccc3oc4ccccc4c3c2)c2cccc3oc4c5ccccc5c(-c5ccc6c7ccccc7n7c8ccccc8c5c67)cc4c23)cc1] | 72% |
| 1-40 | 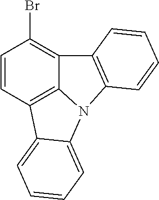 [SMILES: Brc1ccc2c3ccccc3n3c4ccccc4c1c23] | 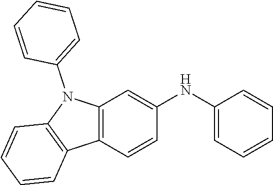 [SMILES: c1ccc(Nc2ccc3c4ccccc4n(-c4ccccc4)c3c2)cc1] | 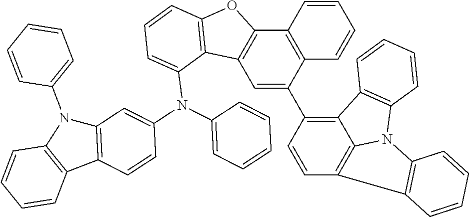 [SMILES: c1ccc(N(c2ccc3c4ccccc4n(-c4ccccc4)c3c2)c2cccc3oc4c5ccccc5c(-c5ccc6c7ccccc7n7c8ccccc8c5c67)cc4c23)cc1] | 72% |
| 1-49 | 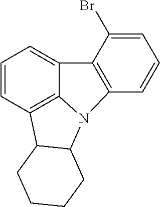 [SMILES: Brc1cccc2c1c1cccc3c1n2C1CCCCC31] | 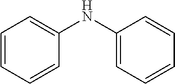 [SMILES: c1ccc(Nc2ccccc2)cc1] | 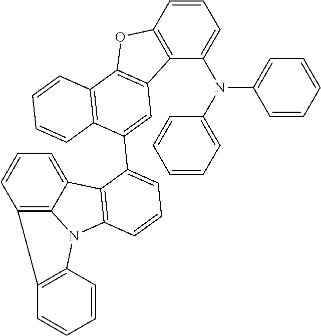 [SMILES: c1ccc(N(c2ccccc2)c2cccc3oc4c5ccccc5c(-c5cccc6c5c5cccc7c8ccccc8n6c75)cc4c23)cc1] | 84% |
| 1-51 | 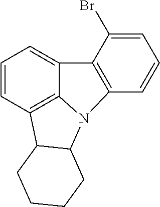 [SMILES: Brc1cccc2c1c1cccc3c1n2C1CCCCC31] | 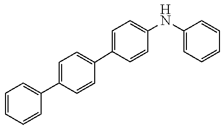 [SMILES: c1ccc(Nc2ccc(-c3ccc(-c4ccccc4)cc3)cc2)cc1] | 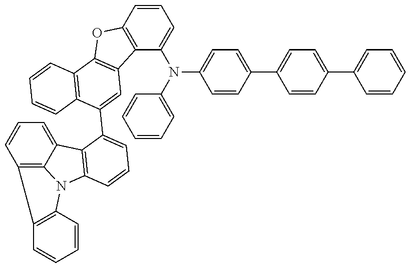 [SMILES: c1ccc(-c2ccc(-c3ccc(N(c4ccccc4)c4cccc5oc6c7ccccc7c(-c7cccc8c7c7cccc9c%10ccccc%10n8c97)cc6c45)cc3)cc2)cc1] | 76% |
| 1-55 | 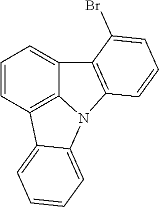 [SMILES: Brc1cccc2c1c1cccc3c4ccccc4n2c31] | 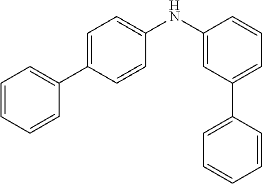 [SMILES: c1ccc(-c2ccc(Nc3cccc(-c4ccccc4)c3)cc2)cc1] | 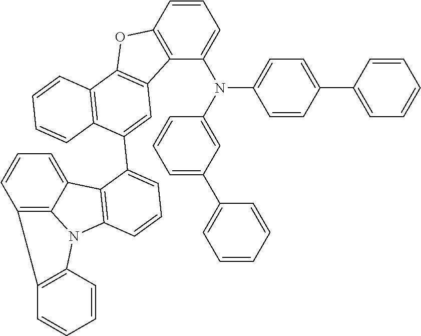 [SMILES: c1ccc(-c2ccc(N(c3cccc(-c4ccccc4)c3)c3cccc4oc5c6ccccc6c(-c6cccc7c6c6cccc8c9ccccc9n7c86)cc5c34)cc2)cc1] | 71% |
| 1-59 | 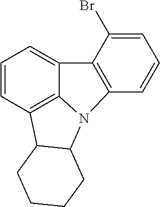 [SMILES: Brc1cccc2c1c1cccc3c1n2C1CCCCC31] | 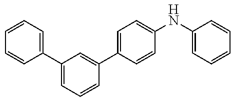 [SMILES: c1ccc(Nc2ccc(-c3cccc(-c4ccccc4)c3)cc2)cc1] | 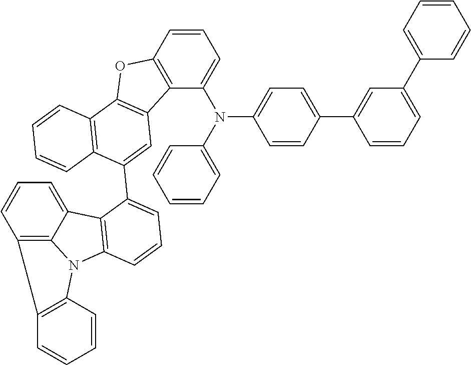 [SMILES: c1ccc(-c2cccc(-c3ccc(N(c4ccccc4)c4cccc5oc6c7ccccc7c(-c7cccc8c7c7cccc9c%10ccccc%10n8c97)cc6c45)cc3)c2)cc1] | 86% |
| 1-65 | 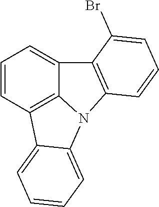 [SMILES: Brc1cccc2c1c1cccc3c4ccccc4n2c31] | 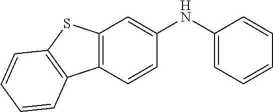 [SMILES: c1ccc(Nc2ccc3c(c2)sc2ccccc23)cc1] | 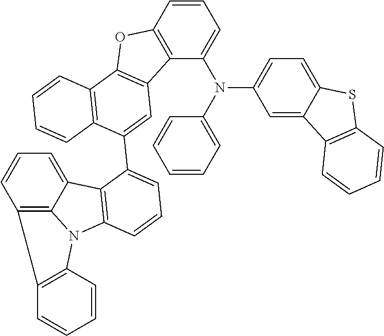 [SMILES: c1ccc(N(c2ccc3sc4ccccc4c3c2)c2cccc3oc4c5ccccc5c(-c5cccc6c5c5cccc7c8ccccc8n6c75)cc4c23)cc1] | 88% |
| 1-73 | 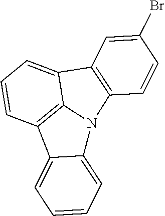 [SMILES: Brc1ccc2c(c1)c1cccc3c4ccccc4n2c31] | 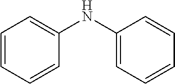 [SMILES: c1ccc(Nc2ccccc2)cc1] | 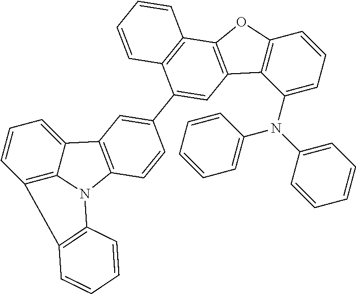 [SMILES: c1ccc(N(c2ccccc2)c2cccc3oc4c5ccccc5c(-c5ccc6c(c5)c5cccc7c8ccccc8n6c75)cc4c23)cc1] | 83% |
| 1-78 | 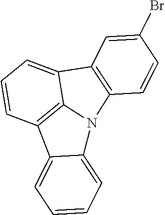 [SMILES: Brc1ccc2c(c1)c1cccc3c4ccccc4n2c31] | 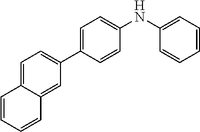 [SMILES: c1ccc(Nc2ccc(-c3ccc4ccccc4c3)cc2)cc1] | 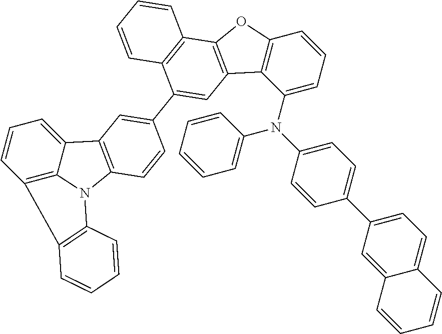 [SMILES: c1ccc(N(c2ccc(-c3ccc4ccccc4c3)cc2)c2cccc3oc4c5ccccc5c(-c5ccc6c(c5)c5cccc7c8ccccc8n6c75)cc4c23)cc1] | 76% |
| 1-88 | 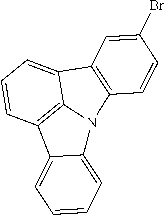 [SMILES: Brc1ccc2c(c1)c1cccc3c4ccccc4n2c31] | 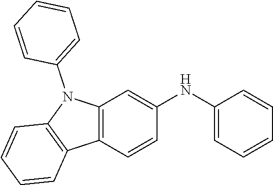 [SMILES: c1ccc(Nc2ccc3c4ccccc4n(-c4ccccc4)c3c2)cc1] | 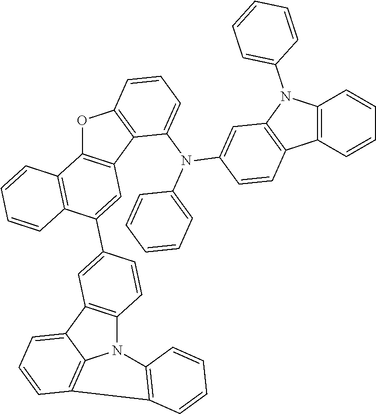 [SMILES: c1ccc(N(c2ccc3c4ccccc4n(-c4ccccc4)c3c2)c2cccc3oc4c5ccccc5c(-c5ccc6c(c5)c5cccc7c8ccccc8n6c75)cc4c23)cc1] | 78% |
| 1-97 | 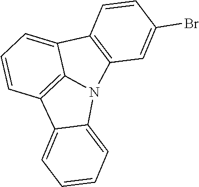 [SMILES: Brc1ccc2c3cccc4c5ccccc5n(c2c1)c43] | 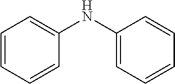 [SMILES: c1ccc(Nc2ccccc2)cc1] | 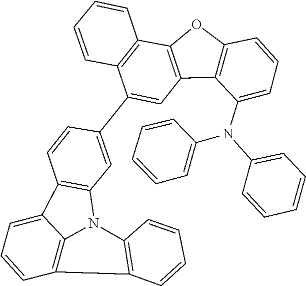 [SMILES: c1ccc(N(c2ccccc2)c2cccc3oc4c5ccccc5c(-c5ccc6c7cccc8c9ccccc9n(c6c5)c87)cc4c23)cc1] | 80% |
| 1-100 | 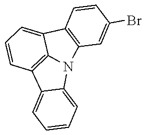 [SMILES: Brc1ccc2c3cccc4c5ccccc5n(c2c1)c43] | 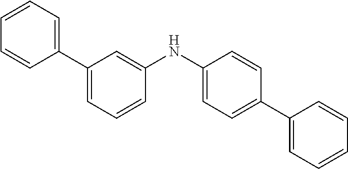 [SMILES: c1ccc(-c2ccc(Nc3cccc(-c4ccccc4)c3)cc2)cc1] | 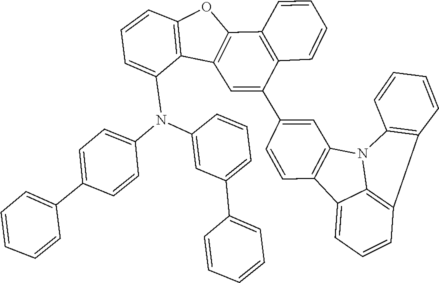 [SMILES: c1ccc(-c2ccc(N(c3cccc(-c4ccccc4)c3)c3cccc4oc5c6ccccc6c(-c6ccc7c8cccc9c%10ccccc%10n(c7c6)c98)cc5c34)cc2)cc1] | 86% |
| 1-106 | 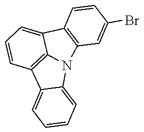 [SMILES: Brc1ccc2c3cccc4c5ccccc5n(c2c1)c43] | 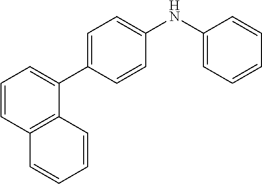 [SMILES: c1ccc(Nc2ccc(-c3cccc4ccccc34)cc2)cc1] | 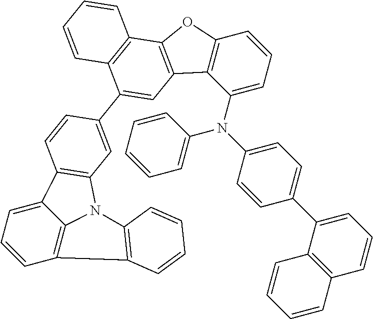 [SMILES: c1ccc(N(c2ccc(-c3cccc4ccccc34)cc2)c2cccc3oc4c5ccccc5c(-c5ccc6c7cccc8c9ccccc9n(c6c5)c87)cc4c23)cc1] | 72% |
| 1-111 | 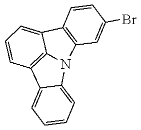 [SMILES: Brc1ccc2c3cccc4c5ccccc5n(c2c1)c43] | 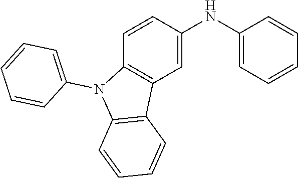 [SMILES: c1ccc(Nc2ccc3c(c2)c2ccccc2n3-c2ccccc2)cc1] | 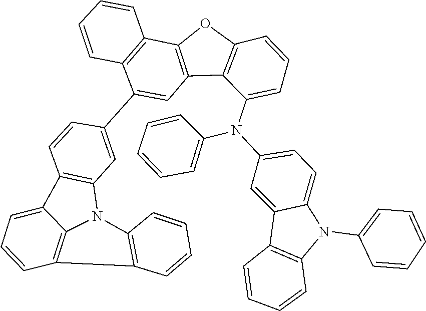 [SMILES: c1ccc(N(c2ccc3c(c2)c2ccccc2n3-c2ccccc2)c2cccc3oc4c5ccccc5c(-c5ccc6c7cccc8c9ccccc9n(c6c5)c87)cc4c23)cc1] | 79% |
| 1-121 | 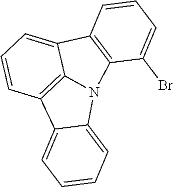 [SMILES: Brc1cccc2c3cccc4c5ccccc5n(c12)c43] | 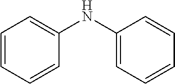 [SMILES: c1ccc(Nc2ccccc2)cc1] | 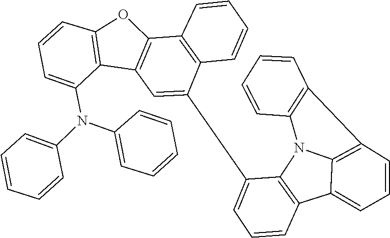 [SMILES: c1ccc(N(c2ccccc2)c2cccc3oc4c5ccccc5c(-c5cccc6c7cccc8c9ccccc9n(c56)c87)cc4c23)cc1] | 81% |
| 1-123 | 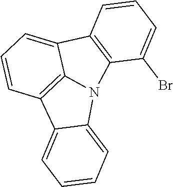 [SMILES: Brc1cccc2c3cccc4c5ccccc5n(c12)c43] | 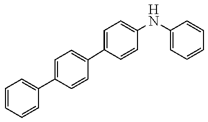 [SMILES: c1ccc(Nc2ccc(-c3ccc(-c4ccccc4)cc3)cc2)cc1] | 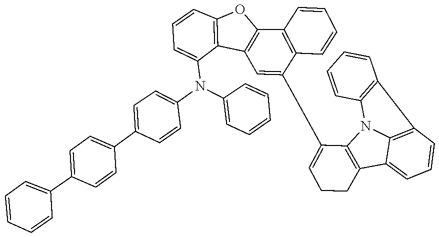 [SMILES: C1=C(c2cc3c(oc4cccc(N(c5ccccc5)c5ccc(-c6ccc(-c7ccccc7)cc6)cc5)c43)c3ccccc23)c2c(c3cccc4c5ccccc5n2c34)CC1] | 70% |
| 1-132 | 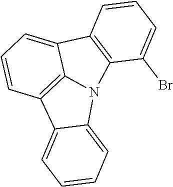 [SMILES: Brc1cccc2c3cccc4c5ccccc5n(c12)c43] | 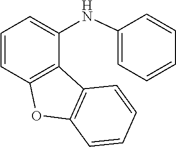 [SMILES: c1ccc(Nc2cccc3oc4ccccc4c23)cc1] | 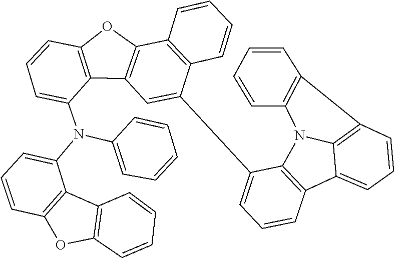 [SMILES: c1ccc(N(c2cccc3oc4ccccc4c23)c2cccc3oc4c5ccccc5c(-c5cccc6c7cccc8c9ccccc9n(c56)c87)cc4c23)cc1] | 70% |
| 1-145 | 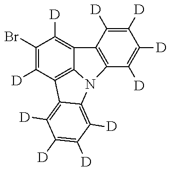 [SMILES: [2H]c1c([2H])c([2H])c2c(c1[2H])c1c([2H])c(Br)c([2H])c3c4c([2H])c([2H])c([2H])c([2H])c4n2c13] | 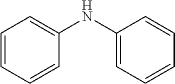 [SMILES: c1ccc(Nc2ccccc2)cc1] | 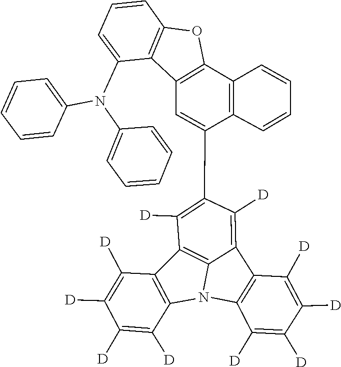 [SMILES: [2H]c1c([2H])c([2H])c2c(c1[2H])c1c([2H])c(-c3cc4c(oc5cccc(N(c6ccccc6)c6ccccc6)c54)c4ccccc34)c([2H])c3c4c([2H])c([2H])c([2H])c([2H])c4n2c13] | 74% |
| 1-160 | 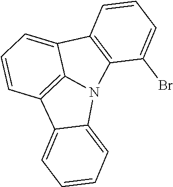 [SMILES: Brc1cccc2c3cccc4c5ccccc5n(c12)c43] | 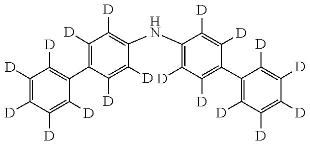 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] | 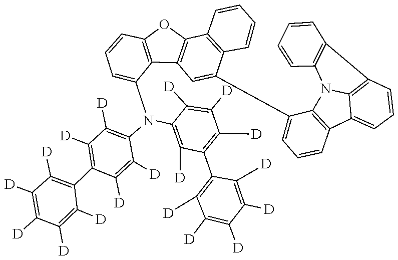 [SMILES: [2H]c1c([2H])c([2H])c(-c2c([2H])c([2H])c(N(c3c([2H])c([2H])c([2H])c(-c4c([2H])c([2H])c([2H])c([2H])c4[2H])c3[2H])c3cccc4oc5c6ccccc6c(-c6cccc7c8cccc9c%10ccccc%10n(c67)c98)cc5c34)c([2H])c2[2H])c([2H])c1[2H]] | 85% |

Target compounds were prepared as in the following Table 2 in the same manner as in Preparation Example 1, except that 8-bromo-1-chloro-dibenzofuiran was used instead of 5-bromo-7-chloronaphtho[1,2-b]benzofuran, Compound C of the following Table 2 was used instead of 2-bromoindolo[3,2,1-jk]carbazole, and Compound D of the following Table 2 was used instead of diphenylamine.

TABLE 2

| Com- | | | | |
|---|---|---|---|---|
| pound | | | | |
| No. | Compound C | Compound D | Target Compound | Yield |
| 2-1 | 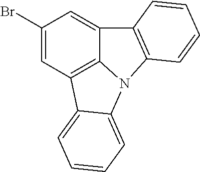 [SMILES: Brc1cc2c3ccccc3n3c4ccccc4c(c1)c23] | 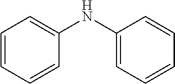 [SMILES: c1ccc(Nc2ccccc2)cc1] | 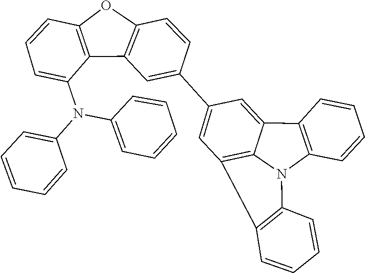 [SMILES: c1ccc(N(c2ccccc2)c2cccc3oc4ccc(-c5cc6c7ccccc7n7c8ccccc8c(c5)c67)cc4c23)cc1] | 76% |
| 2-2 | 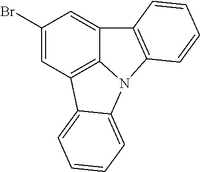 [SMILES: Brc1cc2c3ccccc3n3c4ccccc4c(c1)c23] | 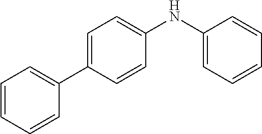 [SMILES: c1ccc(Nc2ccc(-c3ccccc3)cc2)cc1] | 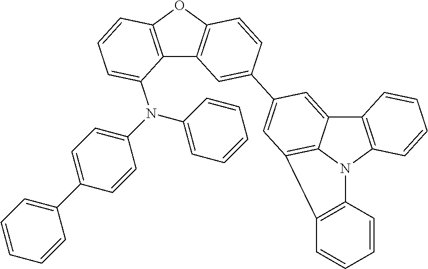 [SMILES: c1ccc(-c2ccc(N(c3ccccc3)c3cccc4oc5ccc(-c6cc7c8ccccc8n8c9ccccc9c(c6)c78)cc5c34)cc2)cc1] | 82% |
| 2-3 | 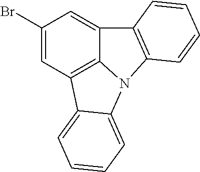 [SMILES: Brc1cc2c3ccccc3n3c4ccccc4c(c1)c23] | 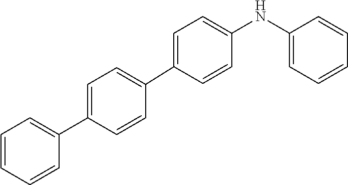 [SMILES: c1ccc(Nc2ccc(-c3ccc(-c4ccccc4)cc3)cc2)cc1] | 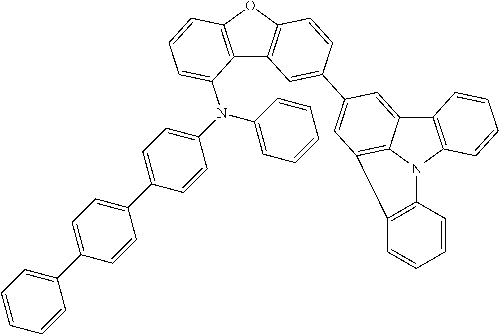 [SMILES: c1ccc(-c2ccc(-c3ccc(N(c4ccccc4)c4cccc5oc6ccc(-c7cc8c9ccccc9n9c%10ccccc%10c(c7)c89)cc6c45)cc3)cc2)cc1] | 78% |
| 2-4 | 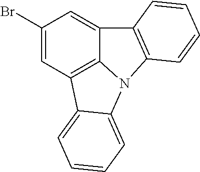 [SMILES: Brc1cc2c3ccccc3n3c4ccccc4c(c1)c23] | 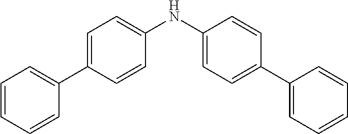 [SMILES: c1ccc(-c2ccc(Nc3ccc(-c4ccccc4)cc3)cc2)cc1] | 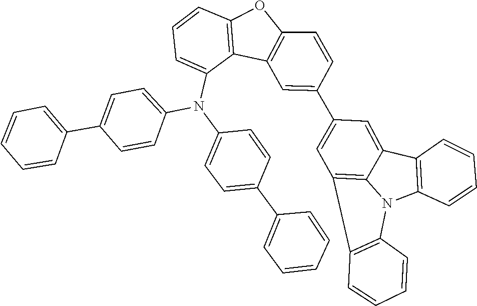 [SMILES: c1ccc(-c2ccc(N(c3ccc(-c4ccccc4)cc3)c3cccc4oc5ccc(-c6cc7c8ccccc8n8c9ccccc9c(c6)c78)cc5c34)cc2)cc1] | 83% |
| 2-5 | 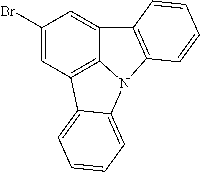 [SMILES: Brc1cc2c3ccccc3n3c4ccccc4c(c1)c23] | 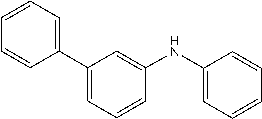 [SMILES: c1ccc(Nc2cccc(-c3ccccc3)c2)cc1] | 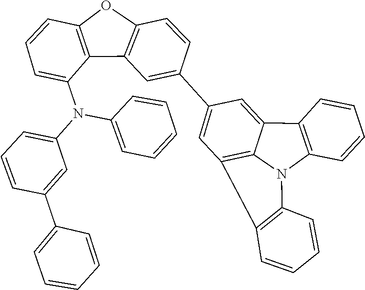 [SMILES: c1ccc(-c2cccc(N(c3ccccc3)c3cccc4oc5ccc(-c6cc7c8ccccc8n8c9ccccc9c(c6)c78)cc5c34)c2)cc1] | 73% |
| 2-6 | 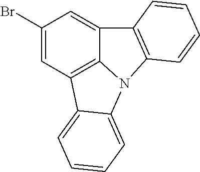 [SMILES: Brc1cc2c3ccccc3n3c4ccccc4c(c1)c23] | 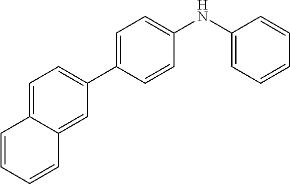 [SMILES: c1ccc(Nc2ccc(-c3ccc4ccccc4c3)cc2)cc1] | 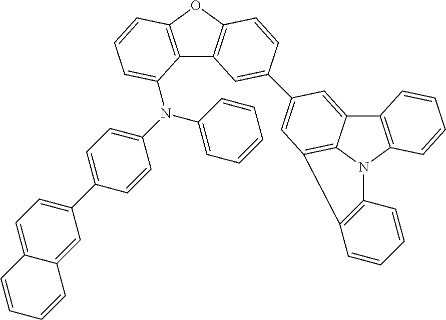 [SMILES: c1ccc(N(c2ccc(-c3ccc4ccccc4c3)cc2)c2cccc3oc4ccc(-c5cc6c7ccccc7n7c8ccccc8c(c5)c67)cc4c23)cc1] | 74% |
| 2-7 | 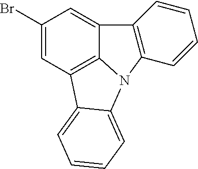 [SMILES: Brc1cc2c3ccccc3n3c4ccccc4c(c1)c23] | 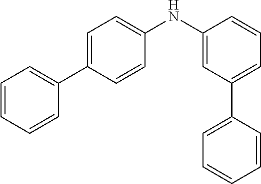 [SMILES: c1ccc(-c2ccc(Nc3cccc(-c4ccccc4)c3)cc2)cc1] | 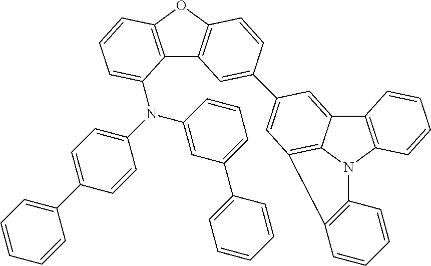 [SMILES: c1ccc(-c2ccc(N(c3cccc(-c4ccccc4)c3)c3cccc4oc5ccc(-c6cc7c8ccccc8n8c9ccccc9c(c6)c78)cc5c34)cc2)cc1] | 77% |
| 2-9 | 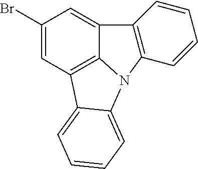 [SMILES: Brc1cc2c3ccccc3n3c4ccccc4c(c1)c23] | 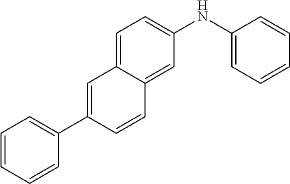 [SMILES: c1ccc(Nc2ccc3cc(-c4ccccc4)ccc3c2)cc1] | 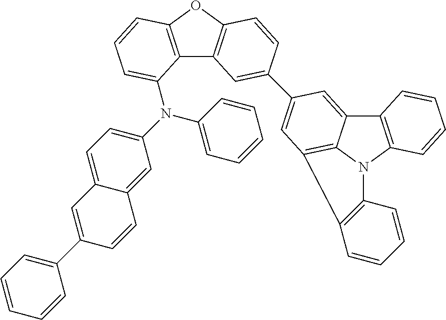 [SMILES: c1ccc(-c2ccc3cc(N(c4ccccc4)c4cccc5oc6ccc(-c7cc8c9ccccc9n9c%10ccccc%10c(c7)c89)cc6c45)ccc3c2)cc1] | 88% |
| 2-10 | 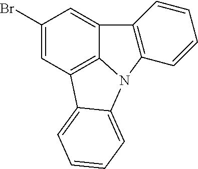 [SMILES: Brc1cc2c3ccccc3n3c4ccccc4c(c1)c23] | 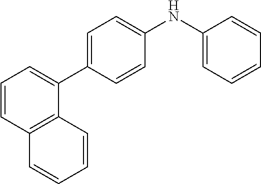 [SMILES: c1ccc(Nc2ccc(-c3cccc4ccccc34)cc2)cc1] | 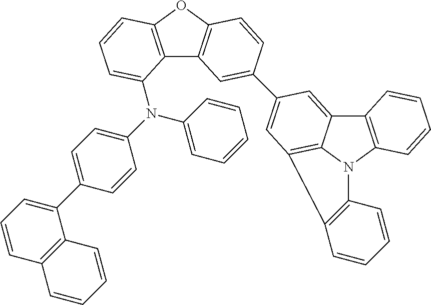 [SMILES: c1ccc(N(c2ccc(-c3cccc4ccccc34)cc2)c2cccc3oc4ccc(-c5cc6c7ccccc7n7c8ccccc8c(c5)c67)cc4c23)cc1] | 76% |
| 2-12 | 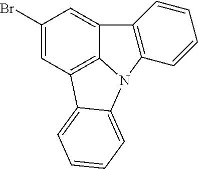 [SMILES: Brc1cc2c3ccccc3n3c4ccccc4c(c1)c23] | 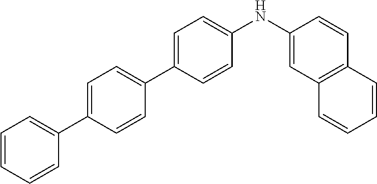 [SMILES: c1ccc(-c2ccc(-c3ccc(Nc4ccc5ccccc5c4)cc3)cc2)cc1] | 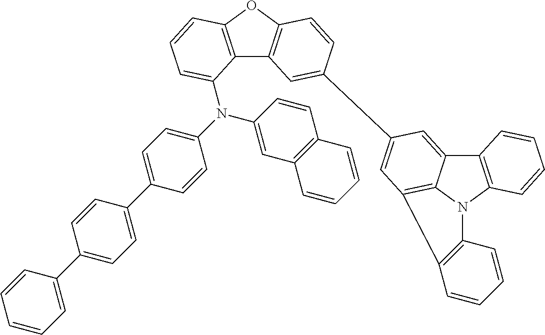 [SMILES: c1ccc(-c2ccc(-c3ccc(N(c4ccc5ccccc5c4)c4cccc5oc6ccc(-c7cc8c9ccccc9n9c%10ccccc%10c(c7)c89)cc6c45)cc3)cc2)cc1] | 81% |
| 2-13 | 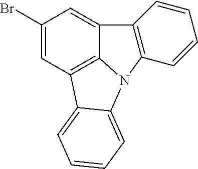 [SMILES: Brc1cc2c3ccccc3n3c4ccccc4c(c1)c23] | 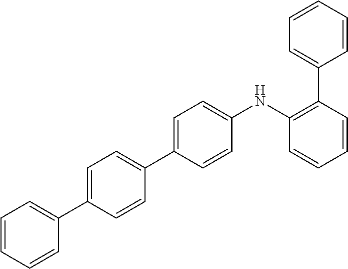 [SMILES: c1ccc(-c2ccc(-c3ccc(Nc4ccccc4-c4ccccc4)cc3)cc2)cc1] | 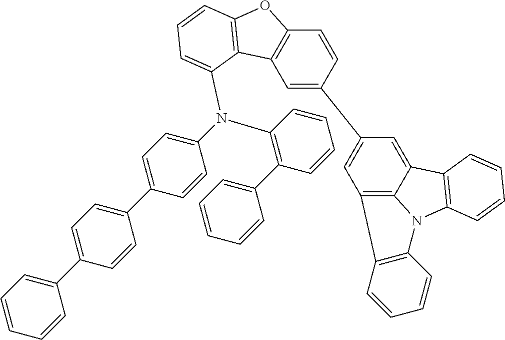 [SMILES: c1ccc(-c2ccc(-c3ccc(N(c4ccccc4-c4ccccc4)c4cccc5oc6ccc(-c7cc8c9ccccc9n9c%10ccccc%10c(c7)c89)cc6c45)cc3)cc2)cc1] | 79% |
| 2-14 | 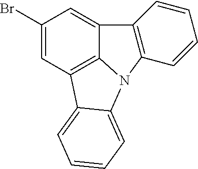 [SMILES: Brc1cc2c3ccccc3n3c4ccccc4c(c1)c23] | 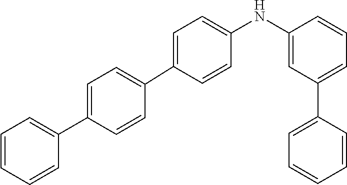 [SMILES: c1ccc(-c2ccc(-c3ccc(Nc4cccc(-c5ccccc5)c4)cc3)cc2)cc1] | 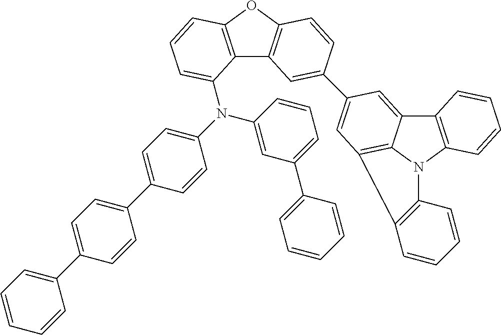 [SMILES: c1ccc(-c2ccc(-c3ccc(N(c4cccc(-c5ccccc5)c4)c4cccc5oc6ccc(-c7cc8c9ccccc9n9c%10ccccc%10c(c7)c89)cc6c45)cc3)cc2)cc1] | 77% |
| 2-15 | 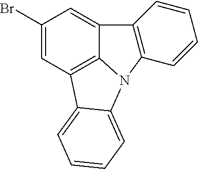 [SMILES: Brc1cc2c3ccccc3n3c4ccccc4c(c1)c23] | 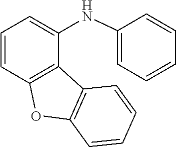 [SMILES: c1ccc(Nc2cccc3oc4ccccc4c23)cc1] | 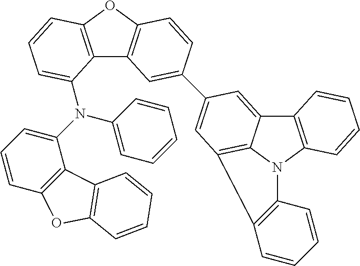 [SMILES: c1ccc(N(c2cccc3oc4ccccc4c23)c2cccc3oc4ccc(-c5cc6c7ccccc7n7c8ccccc8c(c5)c67)cc4c23)cc1] | 86% |
| 2-16 | 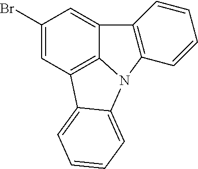 [SMILES: Brc1cc2c3ccccc3n3c4ccccc4c(c1)c23] | 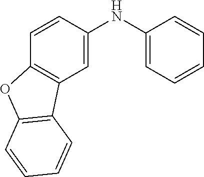 [SMILES: c1ccc(Nc2ccc3oc4ccccc4c3c2)cc1] | 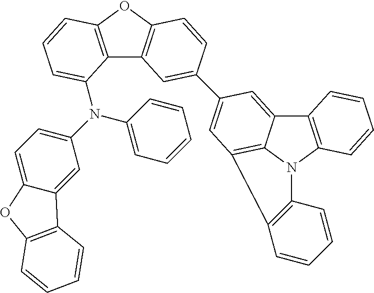 [SMILES: c1ccc(N(c2ccc3oc4ccccc4c3c2)c2cccc3oc4ccc(-c5cc6c7ccccc7n7c8ccccc8c(c5)c67)cc4c23)cc1] | 77% |
| 2-17 | 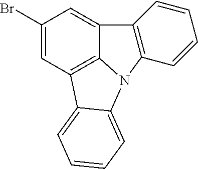 [SMILES: Brc1cc2c3ccccc3n3c4ccccc4c(c1)c23] | 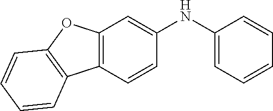 [SMILES: c1ccc(Nc2ccc3c(c2)oc2ccccc23)cc1] | 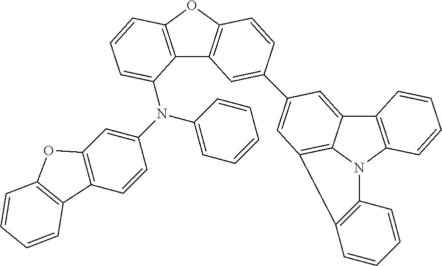 [SMILES: c1ccc(N(c2ccc3c(c2)oc2ccccc23)c2cccc3oc4ccc(-c5cc6c7ccccc7n7c8ccccc8c(c5)c67)cc4c23)cc1] | 71% |
| 2-19 | 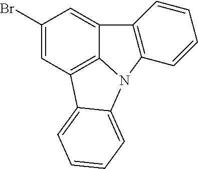 [SMILES: Brc1cc2c3ccccc3n3c4ccccc4c(c1)c23] | 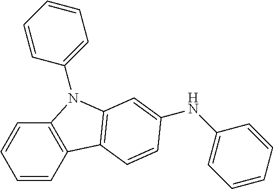 [SMILES: c1ccc(Nc2ccc3c4ccccc4n(-c4ccccc4)c3c2)cc1] | 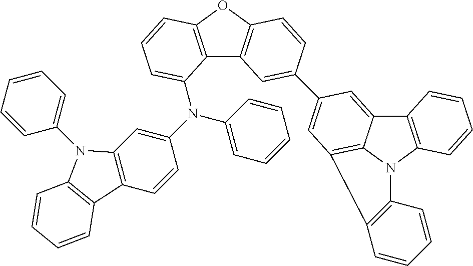 [SMILES: c1ccc(N(c2ccc3c4ccccc4n(-c4ccccc4)c3c2)c2cccc3oc4ccc(-c5cc6c7ccccc7n7c8ccccc8c(c5)c67)cc4c23)cc1] | 83% |
| 2-20 | 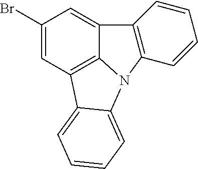 [SMILES: Brc1cc2c3ccccc3n3c4ccccc4c(c1)c23] | 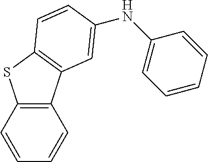 [SMILES: c1ccc(Nc2ccc3sc4ccccc4c3c2)cc1] | 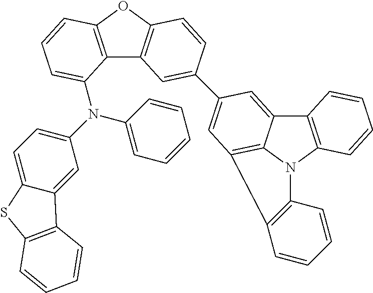 [SMILES: c1ccc(N(c2ccc3sc4ccccc4c3c2)c2cccc3oc4ccc(-c5cc6c7ccccc7n7c8ccccc8c(c5)c67)cc4c23)cc1] | 71% |
| 2-29 | 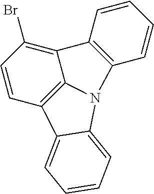 [SMILES: Brc1ccc2c3ccccc3n3c4ccccc4c1c23] | 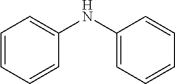 [SMILES: c1ccc(Nc2ccccc2)cc1] | 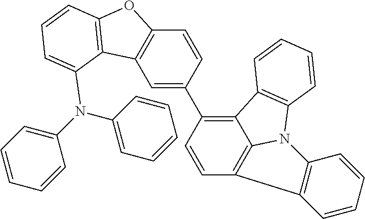 [SMILES: c1ccc(N(c2ccccc2)c2cccc3oc4ccc(-c5ccc6c7ccccc7n7c8ccccc8c5c67)cc4c23)cc1] | 78% |
| 2-31 | 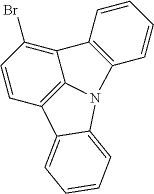 [SMILES: Brc1ccc2c3ccccc3n3c4ccccc4c1c23] | 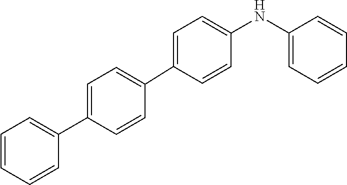 [SMILES: c1ccc(Nc2ccc(-c3ccc(-c4ccccc4)cc3)cc2)cc1] | 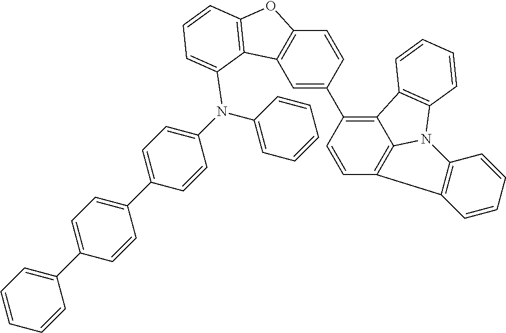 [SMILES: c1ccc(-c2ccc(-c3ccc(N(c4ccccc4)c4cccc5oc6ccc(-c7ccc8c9ccccc9n9c%10ccccc%10c7c89)cc6c45)cc3)cc2)cc1] | 82% |
| 2-38 | 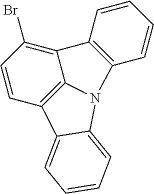 [SMILES: Brc1ccc2c3ccccc3n3c4ccccc4c1c23] | 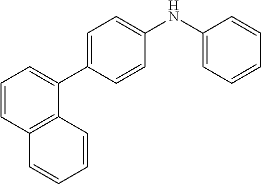 [SMILES: c1ccc(Nc2ccc(-c3cccc4ccccc34)cc2)cc1] | 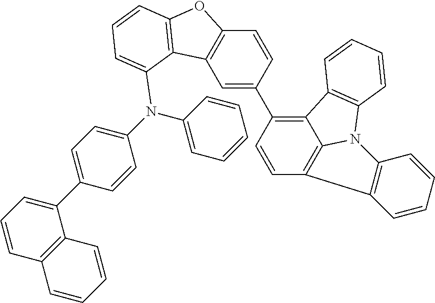 [SMILES: c1ccc(N(c2ccc(-c3cccc4ccccc34)cc2)c2cccc3oc4ccc(-c5ccc6c7ccccc7n7c8ccccc8c5c67)cc4c23)cc1] | 84% |
| 2-46 | 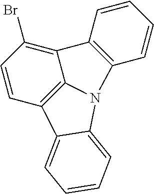 [SMILES: Brc1ccc2c3ccccc3n3c4ccccc4c1c23] | 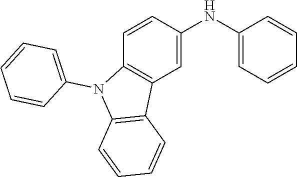 [SMILES: c1ccc(Nc2ccc3c(c2)c2ccccc2n3-c2ccccc2)cc1] | 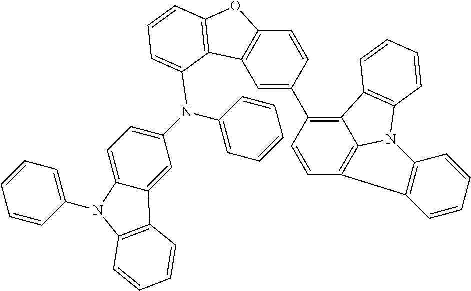 [SMILES: c1ccc(N(c2ccc3c(c2)c2ccccc2n3-c2ccccc2)c2cccc3oc4ccc(-c5ccc6c7ccccc7n7c8ccccc8c5c67)cc4c23)cc1] | 78% |
| 2-57 | 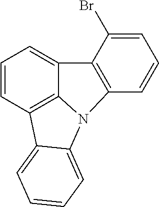 [SMILES: Brc1cccc2c1c1cccc3c4ccccc4n2c31] | 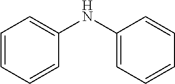 [SMILES: c1ccc(Nc2ccccc2)cc1] | 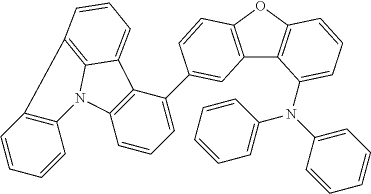 [SMILES: c1ccc(N(c2ccccc2)c2cccc3oc4ccc(-c5cccc6c5c5cccc7c8ccccc8n6c75)cc4c23)cc1] | 83% |
| 2-60 | 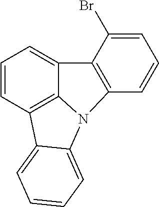 [SMILES: Brc1cccc2c1c1cccc3c4ccccc4n2c31] | 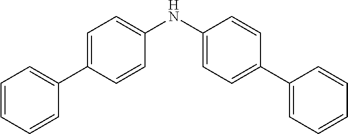 [SMILES: c1ccc(-c2ccc(Nc3ccc(-c4ccccc4)cc3)cc2)cc1] | 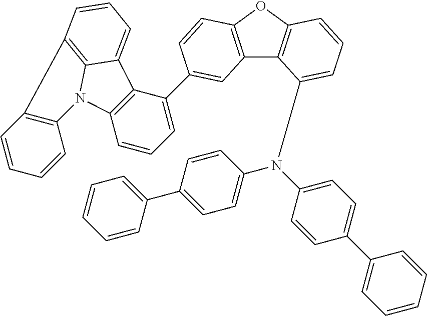 [SMILES: c1ccc(-c2ccc(N(c3ccc(-c4ccccc4)cc3)c3cccc4oc5ccc(-c6cccc7c6c6cccc8c9ccccc9n7c86)cc5c34)cc2)cc1] | 70% |
| 2-68 | 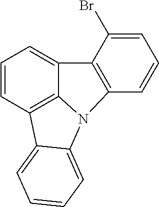 [SMILES: Brc1cccc2c1c1cccc3c4ccccc4n2c31] | 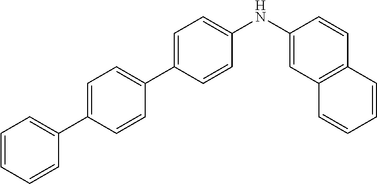 [SMILES: c1ccc(-c2ccc(-c3ccc(Nc4ccc5ccccc5c4)cc3)cc2)cc1] | 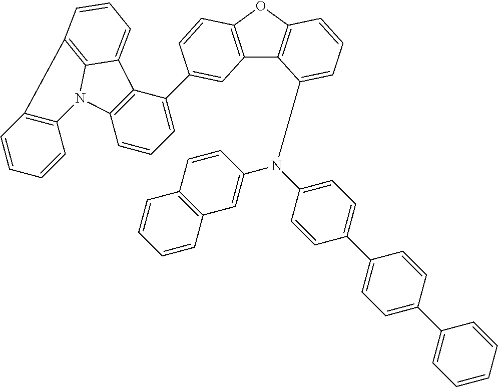 [SMILES: c1ccc(-c2ccc(-c3ccc(N(c4ccc5ccccc5c4)c4cccc5oc6ccc(-c7cccc8c7c7cccc9c%10ccccc%10n8c97)cc6c45)cc3)cc2)cc1] | 73% |
| 2-70 | 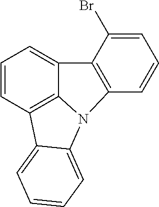 [SMILES: Brc1cccc2c1c1cccc3c4ccccc4n2c31] | 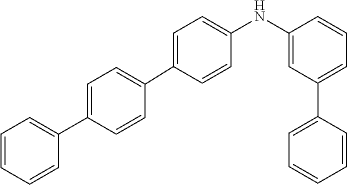 [SMILES: c1ccc(-c2ccc(-c3ccc(Nc4cccc(-c5ccccc5)c4)cc3)cc2)cc1] | 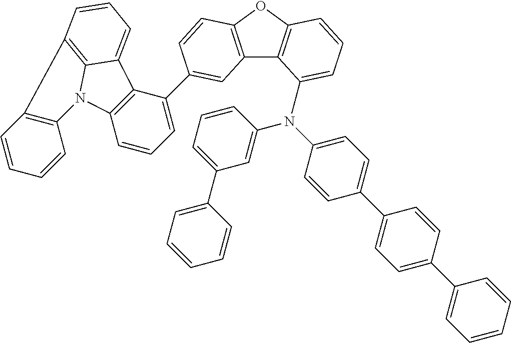 [SMILES: c1ccc(-c2ccc(-c3ccc(N(c4cccc(-c5ccccc5)c4)c4cccc5oc6ccc(-c7cccc8c7c7cccc9c%10ccccc%10n8c97)cc6c45)cc3)cc2)cc1] | 79% |
| 2-74 | 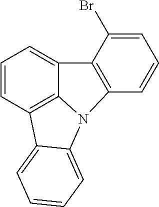 [SMILES: Brc1cccc2c1c1cccc3c4ccccc4n2c31] | 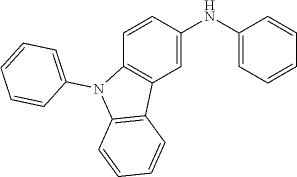 [SMILES: c1ccc(Nc2ccc3c(c2)c2ccccc2n3-c2ccccc2)cc1] | 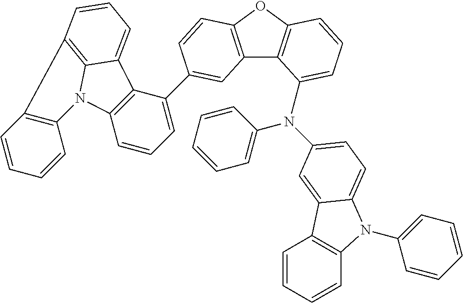 [SMILES: c1ccc(N(c2ccc3c(c2)c2ccccc2n3-c2ccccc2)c2cccc3oc4ccc(-c5cccc6c5c5cccc7c8ccccc8n6c75)cc4c23)cc1] | 88% |
| 2-85 | 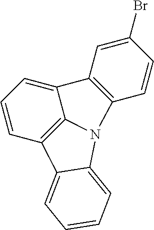 [SMILES: Brc1ccc2c(c1)c1cccc3c4ccccc4n2c31] | 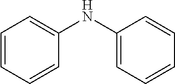 [SMILES: c1ccc(Nc2ccccc2)cc1] | 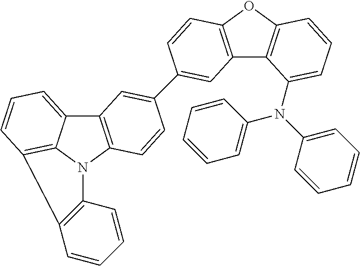 [SMILES: c1ccc(N(c2ccccc2)c2cccc3oc4ccc(-c5ccc6c(c5)c5cccc7c8ccccc8n6c75)cc4c23)cc1] | 88% |
| 2-91 | 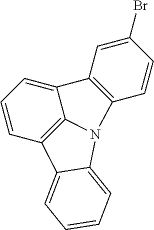 [SMILES: Brc1ccc2c(c1)c1cccc3c4ccccc4n2c31] | 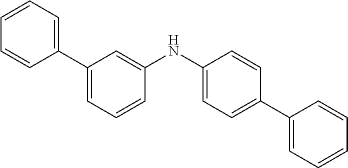 [SMILES: c1ccc(-c2ccc(Nc3cccc(-c4ccccc4)c3)cc2)cc1] | 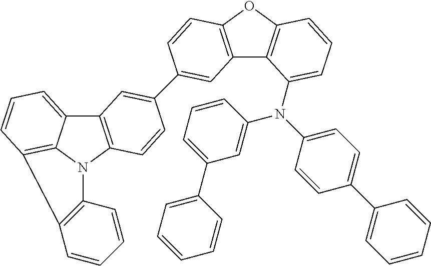 [SMILES: c1ccc(-c2ccc(N(c3cccc(-c4ccccc4)c3)c3cccc4oc5ccc(-c6ccc7c(c6)c6cccc8c9ccccc9n7c86)cc5c34)cc2)cc1] | 70% |
| 2-100 | 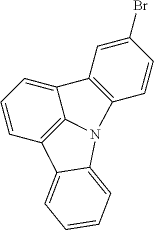 [SMILES: Brc1ccc2c(c1)c1cccc3c4ccccc4n2c31] | 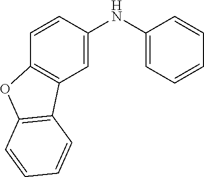 [SMILES: c1ccc(Nc2ccc3oc4ccccc4c3c2)cc1] | 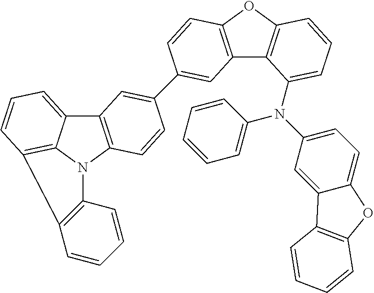 [SMILES: c1ccc(N(c2ccc3oc4ccccc4c3c2)c2cccc3oc4ccc(-c5ccc6c(c5)c5cccc7c8ccccc8n6c75)cc4c23)cc1] | 78% |
| 2-105 | 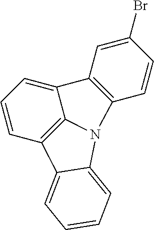 [SMILES: Brc1ccc2c(c1)c1cccc3c4ccccc4n2c31] | 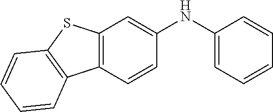 [SMILES: c1ccc(Nc2ccc3c(c2)sc2ccccc23)cc1] | 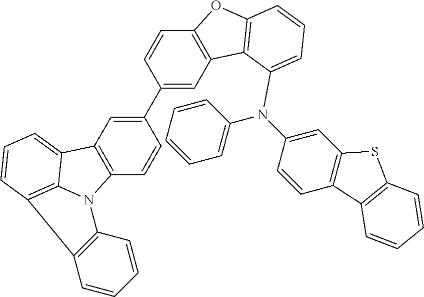 [SMILES: c1ccc(N(c2ccc3c(c2)sc2ccccc23)c2cccc3oc4ccc(-c5ccc6c(c5)c5cccc7c8ccccc8n6c75)cc4c23)cc1] | 72% |
| 2-113 | 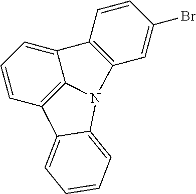 [SMILES: Brc1ccc2c3cccc4c5ccccc5n(c2c1)c43] | 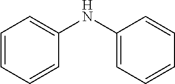 [SMILES: c1ccc(Nc2ccccc2)cc1] | 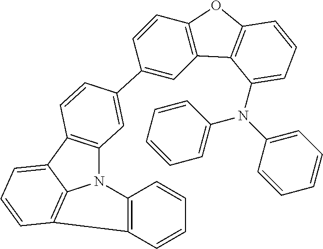 [SMILES: c1ccc(N(c2ccccc2)c2cccc3oc4ccc(-c5ccc6c7cccc8c9ccccc9n(c6c5)c87)cc4c23)cc1] | 84% |
| 2-120 | 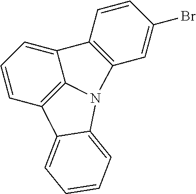 [SMILES: Brc1ccc2c3cccc4c5ccccc5n(c2c1)c43] | 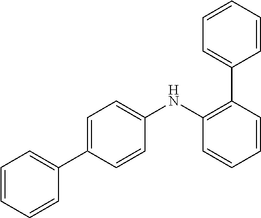 [SMILES: c1ccc(-c2ccc(Nc3ccccc3-c3ccccc3)cc2)cc1] | 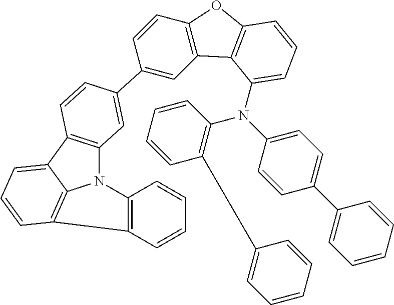 [SMILES: c1ccc(-c2ccc(N(c3ccccc3-c3ccccc3)c3cccc4oc5ccc(-c6ccc7c8cccc9c%10ccccc%10n(c7c6)c98)cc5c34)cc2)cc1] | 77% |
| 2-126 | 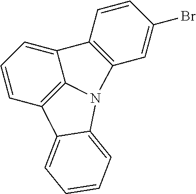 [SMILES: Brc1ccc2c3cccc4c5ccccc5n(c2c1)c43] | 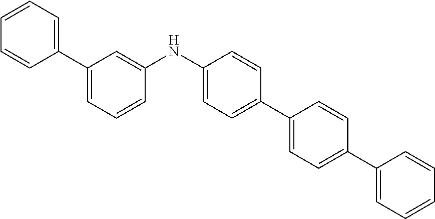 [SMILES: c1ccc(-c2ccc(-c3ccc(Nc4cccc(-c5ccccc5)c4)cc3)cc2)cc1] | 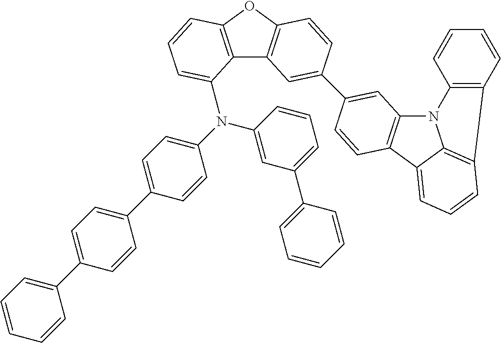 [SMILES: c1ccc(-c2ccc(-c3ccc(N(c4cccc(-c5ccccc5)c4)c4cccc5oc6ccc(-c7ccc8c9cccc%10c%11ccccc%11n(c8c7)c%109)cc6c45)cc3)cc2)cc1] | 88% |
| 2-131 | 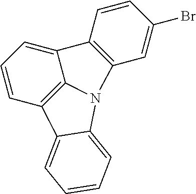 [SMILES: Brc1ccc2c3cccc4c5ccccc5n(c2c1)c43] | 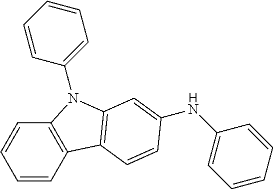 [SMILES: c1ccc(Nc2ccc3c4ccccc4n(-c4ccccc4)c3c2)cc1] | 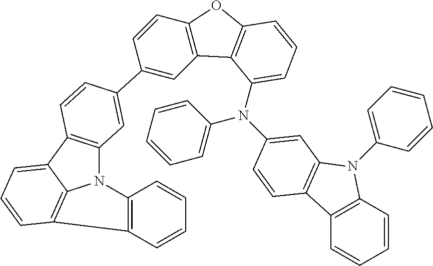 [SMILES: c1ccc(N(c2ccc3c4ccccc4n(-c4ccccc4)c3c2)c2cccc3oc4ccc(-c5ccc6c7cccc8c9ccccc9n(c6c5)c87)cc4c23)cc1] | 71% |
| 2-141 | 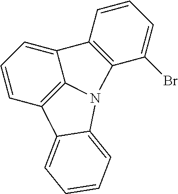 [SMILES: Brc1cccc2c3cccc4c5ccccc5n(c12)c43] | 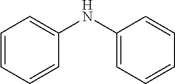 [SMILES: c1ccc(Nc2ccccc2)cc1] | 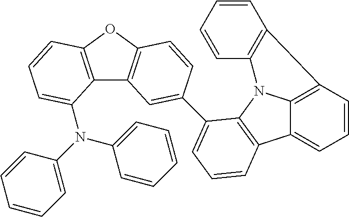 [SMILES: c1ccc(N(c2ccccc2)c2cccc3oc4ccc(-c5cccc6c7cccc8c9ccccc9n(c56)c87)cc4c23)cc1] | 79% |
| 2-147 | 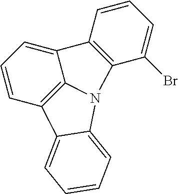 [SMILES: Brc1cccc2c3cccc4c5ccccc5n(c12)c43] | 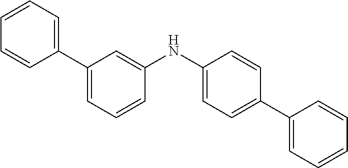 [SMILES: c1ccc(-c2ccc(Nc3cccc(-c4ccccc4)c3)cc2)cc1] | 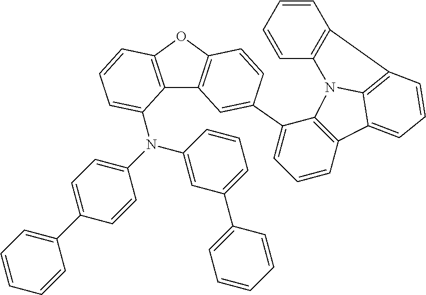 [SMILES: c1ccc(-c2ccc(N(c3cccc(-c4ccccc4)c3)c3cccc4oc5ccc(-c6cccc7c8cccc9c%10ccccc%10n(c67)c98)cc5c34)cc2)cc1] | 79% |
| 2-160 | 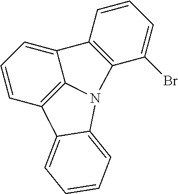 [SMILES: Brc1cccc2c3cccc4c5ccccc5n(c12)c43] | 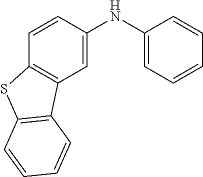 [SMILES: c1ccc(Nc2ccc3sc4ccccc4c3c2)cc1] | 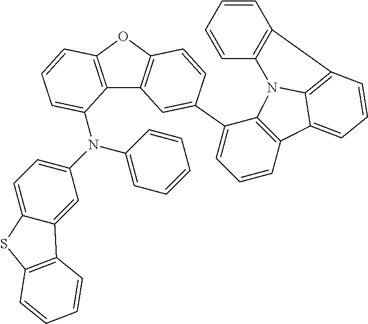 [SMILES: c1ccc(N(c2ccc3sc4ccccc4c3c2)c2cccc3oc4ccc(-c5cccc6c7cccc8c9ccccc9n(c56)c87)cc4c23)cc1] | 78% |
| 2-161 | 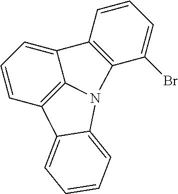 [SMILES: Brc1cccc2c3cccc4c5ccccc5n(c12)c43] | 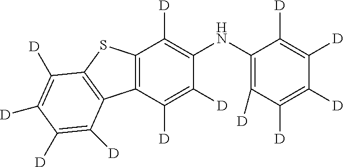 [SMILES: [2H]c1c([2H])c([2H])c(Nc2c([2H])c([2H])c3c(sc4c([2H])c([2H])c([2H])c([2H])c43)c2[2H])c([2H])c1[2H]] | 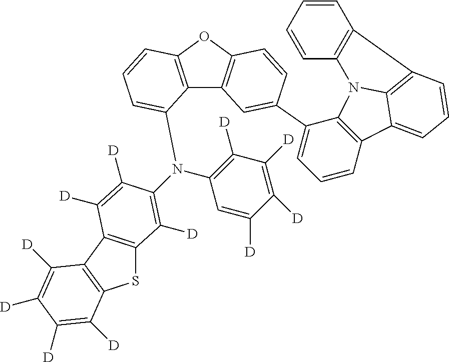 [SMILES: [2H]c1cc(N(c2c([2H])c([2H])c3c(sc4c([2H])c([2H])c([2H])c([2H])c43)c2[2H])c2cccc3oc4ccc(-c5cccc6c7cccc8c9ccccc9n(c56)c87)cc4c23)c([2H])c([2H])c1[2H]] | 82% |

Target compounds were prepared as in the following Table 3 in the same manner as in Preparation Example 1, except that 1-bromo-8-chloro-dibenzofuiran was used instead of 5-bromo-7-chloronaphtho[1,2-b]benzofuran, Compound E of the following Table 3 was used instead of 2-bromoindolo[3,2,1-jk]carbazole, and Compound F of the following Table 3 was used instead of diphenylamine.

TABLE 3

| Com- | | | | |
|---|---|---|---|---|
| pound | | | | |
| No. | Compound E | Compound F | Target Compound | Yield |
| 2-57 | 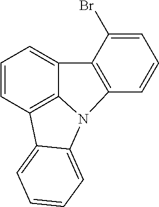 [SMILES: Brc1cccc2c1c1cccc3c4ccccc4n2c31] | 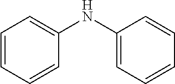 [SMILES: c1ccc(Nc2ccccc2)cc1] | 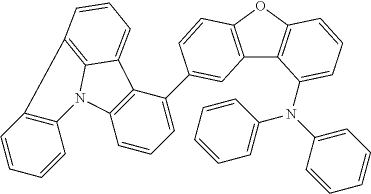 [SMILES: c1ccc(N(c2ccccc2)c2cccc3oc4ccc(-c5cccc6c5c5cccc7c8ccccc8n6c75)cc4c23)cc1] | 83% |
| 2-60 | 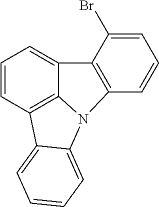 [SMILES: Brc1cccc2c1c1cccc3c4ccccc4n2c31] | 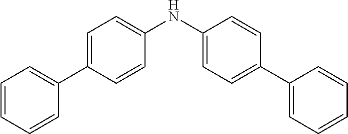 [SMILES: c1ccc(-c2ccc(Nc3ccc(-c4ccccc4)cc3)cc2)cc1] | 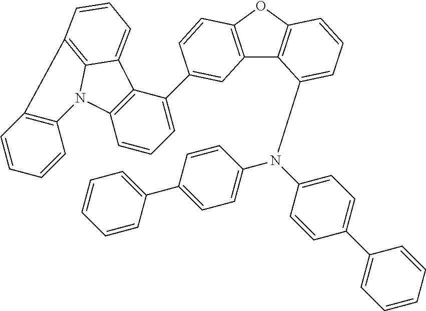 [SMILES: c1ccc(-c2ccc(N(c3ccc(-c4ccccc4)cc3)c3cccc4oc5ccc(-c6cccc7c6c6cccc8c9ccccc9n7c86)cc5c34)cc2)cc1] | 70% |
| 2-68 | 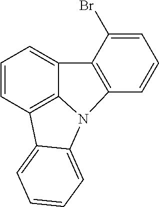 [SMILES: Brc1cccc2c1c1cccc3c4ccccc4n2c31] | 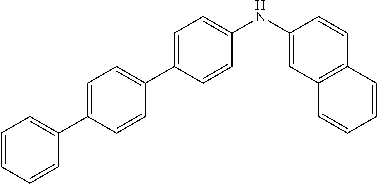 [SMILES: c1ccc(-c2ccc(-c3ccc(Nc4ccc5ccccc5c4)cc3)cc2)cc1] | 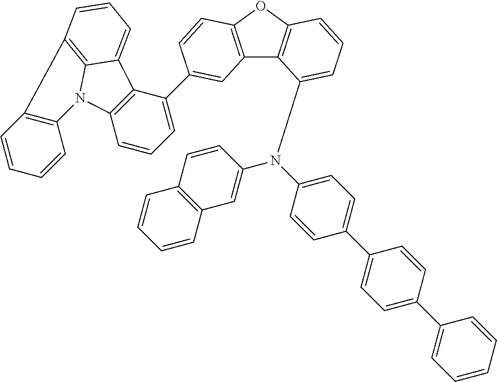 [SMILES: c1ccc(-c2ccc(-c3ccc(N(c4ccc5ccccc5c4)c4cccc5oc6ccc(-c7cccc8c7c7cccc9c%10ccccc%10n8c97)cc6c45)cc3)cc2)cc1] | 73% |
| 2-70 | 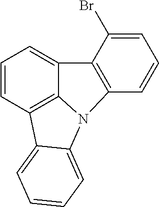 [SMILES: Brc1cccc2c1c1cccc3c4ccccc4n2c31] | 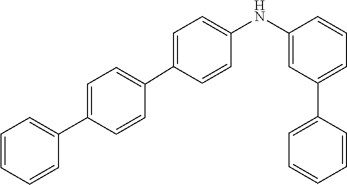 [SMILES: c1ccc(-c2ccc(-c3ccc(Nc4cccc(-c5ccccc5)c4)cc3)cc2)cc1] | 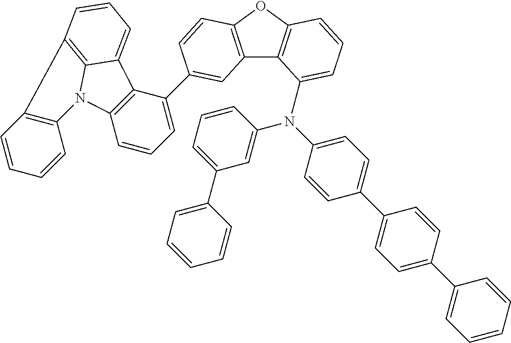 [SMILES: c1ccc(-c2ccc(-c3ccc(N(c4cccc(-c5ccccc5)c4)c4cccc5oc6ccc(-c7cccc8c7c7cccc9c%10ccccc%10n8c97)cc6c45)cc3)cc2)cc1] | 79% |
| 2-74 | 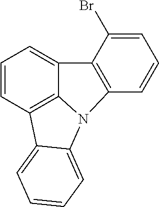 [SMILES: Brc1cccc2c1c1cccc3c4ccccc4n2c31] | 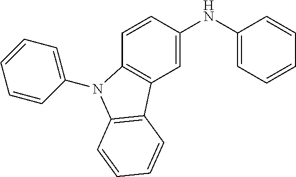 [SMILES: c1ccc(Nc2ccc3c(c2)c2ccccc2n3-c2ccccc2)cc1] | 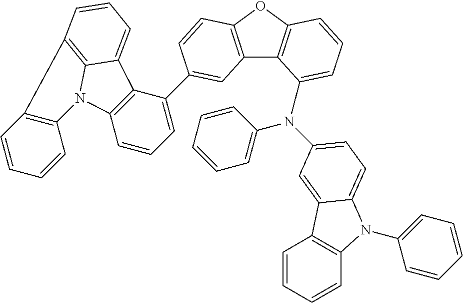 [SMILES: c1ccc(N(c2ccc3c(c2)c2ccccc2n3-c2ccccc2)c2cccc3oc4ccc(-c5cccc6c5c5cccc7c8ccccc8n6c75)cc4c23)cc1] | 88% |
| 2-85 | 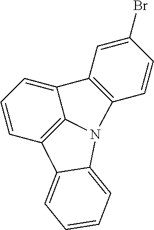 [SMILES: Brc1ccc2c(c1)c1cccc3c4ccccc4n2c31] | 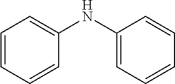 [SMILES: c1ccc(Nc2ccccc2)cc1] | 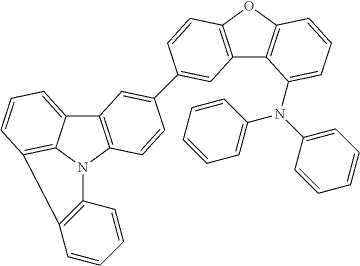 [SMILES: c1ccc(N(c2ccccc2)c2cccc3oc4ccc(-c5ccc6c(c5)c5cccc7c8ccccc8n6c75)cc4c23)cc1] | 88% |
| 2-91 | 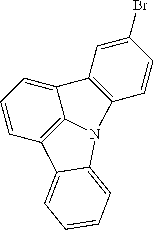 [SMILES: Brc1ccc2c(c1)c1cccc3c4ccccc4n2c31] | 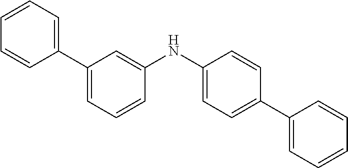 [SMILES: c1ccc(-c2ccc(Nc3cccc(-c4ccccc4)c3)cc2)cc1] | 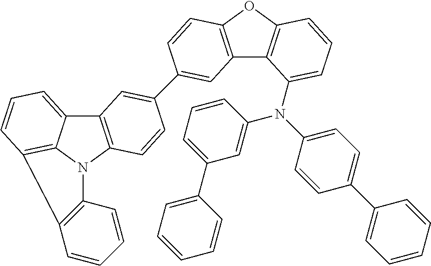 [SMILES: c1ccc(-c2ccc(N(c3cccc(-c4ccccc4)c3)c3cccc4oc5ccc(-c6ccc7c(c6)c6cccc8c9ccccc9n7c86)cc5c34)cc2)cc1] | 70% |
| 2-100 | 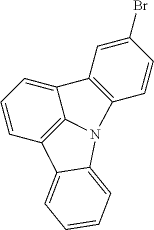 [SMILES: Brc1ccc2c(c1)c1cccc3c4ccccc4n2c31] | 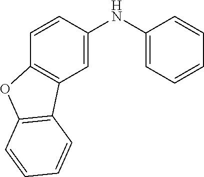 [SMILES: c1ccc(Nc2ccc3oc4ccccc4c3c2)cc1] | 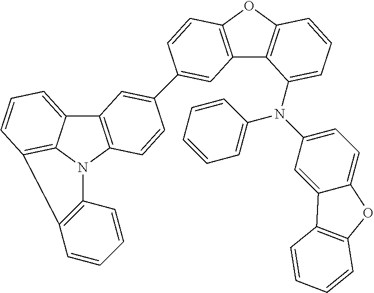 [SMILES: c1ccc(N(c2ccc3oc4ccccc4c3c2)c2cccc3oc4ccc(-c5ccc6c(c5)c5cccc7c8ccccc8n6c75)cc4c23)cc1] | 78% |
| 2-105 | 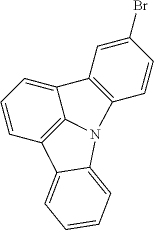 [SMILES: Brc1ccc2c(c1)c1cccc3c4ccccc4n2c31] | 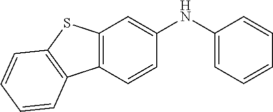 [SMILES: c1ccc(Nc2ccc3c(c2)sc2ccccc23)cc1] | 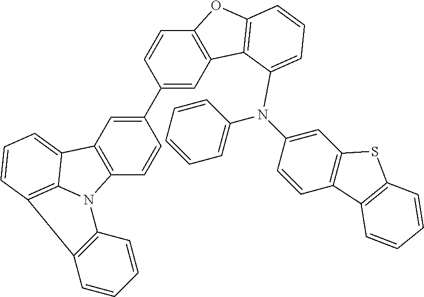 [SMILES: c1ccc(N(c2ccc3c(c2)sc2ccccc23)c2cccc3oc4ccc(-c5ccc6c(c5)c5cccc7c8ccccc8n6c75)cc4c23)cc1] | 72% |
| 2-113 | 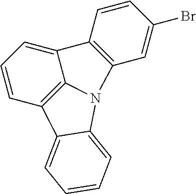 [SMILES: Brc1ccc2c3cccc4c5ccccc5n(c2c1)c43] | 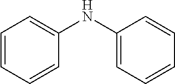 [SMILES: c1ccc(Nc2ccccc2)cc1] | 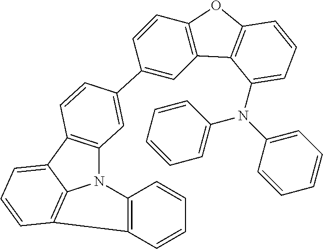 [SMILES: c1ccc(N(c2ccccc2)c2cccc3oc4ccc(-c5ccc6c7cccc8c9ccccc9n(c6c5)c87)cc4c23)cc1] | 84% |
| 2-120 | 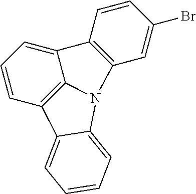 [SMILES: Brc1ccc2c3cccc4c5ccccc5n(c2c1)c43] | 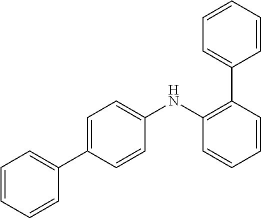 [SMILES: c1ccc(-c2ccc(Nc3ccccc3-c3ccccc3)cc2)cc1] | 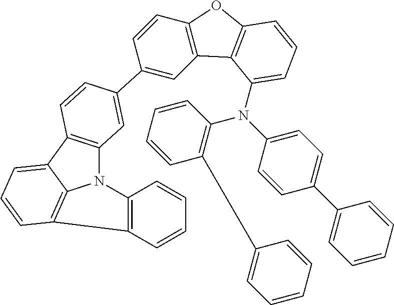 [SMILES: c1ccc(-c2ccc(N(c3ccccc3-c3ccccc3)c3cccc4oc5ccc(-c6ccc7c8cccc9c%10ccccc%10n(c7c6)c98)cc5c34)cc2)cc1] | 77% |
| 2-131 | 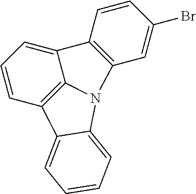 [SMILES: Brc1ccc2c3cccc4c5ccccc5n(c2c1)c43] | 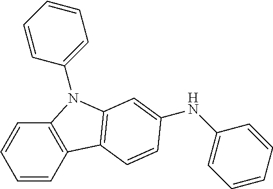 [SMILES: c1ccc(Nc2ccc3c4ccccc4n(-c4ccccc4)c3c2)cc1] | 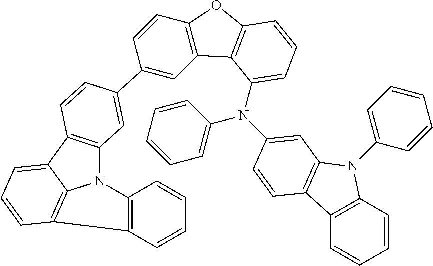 [SMILES: c1ccc(N(c2ccc3c4ccccc4n(-c4ccccc4)c3c2)c2cccc3oc4ccc(-c5ccc6c7cccc8c9ccccc9n(c6c5)c87)cc4c23)cc1] | 71% |

### Preparation Example 2. Preparation of Compound 1-19

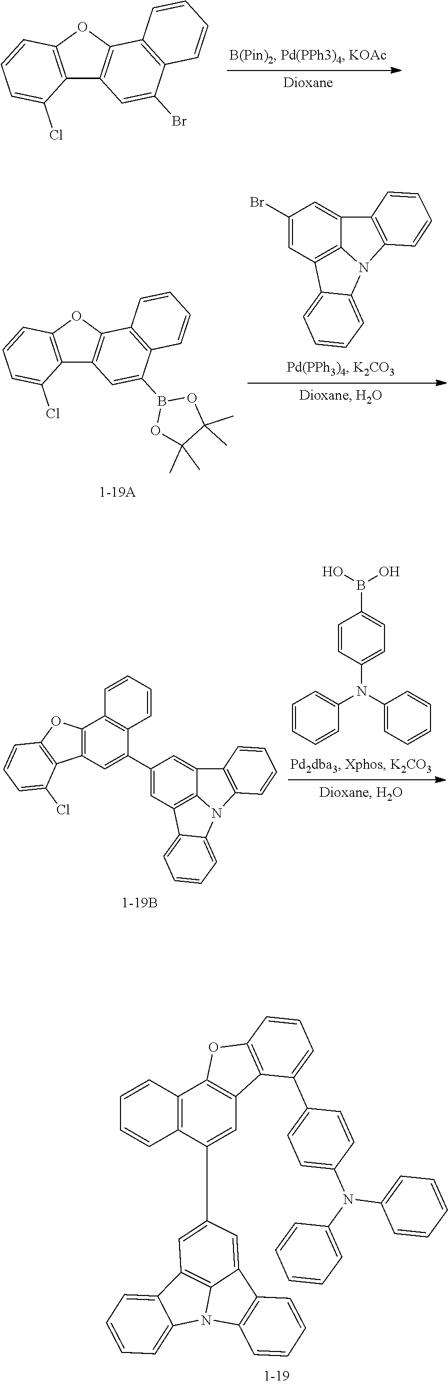

### Preparation Example 2-1. Preparation of Compound 1-19A

5-bromo-7-chloronaphtho[1,2-b]benzofuran (10.0 g, 30.3 mmol), bis(pinacolato)diboron (B(Pin)₂) (11.6 g, 45.5 mmol), Pd(PPh₃)₄ (1.8 g, 1.5 mmol) and KOAc (5.9 g, 60.6 mmol) were dissolved in 1,4-dioxane (100 mL), and then the mixture was refluxed for 3 hours.

After the reaction was completed, the reaction material was filtered under reduced pressure at room temperature, and then the solvent of the filtrate was removed using a rotary evaporator. After that, the result was dissolved in dichloromethane (DCM) and purified by silica, and then recrystallized with dichloromethane and methanol to obtain Compound 1-19A (10.3 g, yield 90%).

### Preparation Example 2-2. Preparation of Compound 1-19B

Compound 1-19A (10.3 g, 27.3 mmol), 2-bromoindolo[3,2,1-jk]carbazole (10.5 g, 32.7 mmol), Pd(PPh₃)₄ (1.6 g, 1.4 mmol) and K₂CO₃ (7.5 g, 54.6 mmol) were dissolved in 1,4-dioxane (100 mL) and distilled water (20 mL), and then the mixture was refluxed for 3 hours.

After the reaction was completed, the reaction material was filtered under reduced pressure at room temperature. After that, the result was dissolved in 1,2-dichlorobenzene (DCB) and purified by silica, and then recrystallized with dichloromethane and methanol to obtain Compound 1-19B (11.1 g, yield 81%).

### Preparation Example 2-3. Preparation of Compound 1-19

Compound 1-19B (11.1 g, 22.7 mmol), (4-(diphenylamino)phenyl)boronic acid (6.6 g, 22.7 mmol), Pd₂(dba)₃ (1.0 g, 1.1 mmol), Xphos (1.1 g, 2.3 mmol) and K₂CO₃ (6.3 g, 45.4 mmol) were dissolved in 1,4-dioxane (110 mL) and distilled water (20 mL), and then the mixture was refluxed for 3 hours.

After the reaction was completed, the reaction material was filtered under reduced pressure at room temperature. After that, the result was dissolved in 1,2-dichlorobenzene and purified by silica, and then recrystallized with 1,2-dichlorobenzene and methanol to obtain target Compound 1-19 (13.2 g, yield 83%).

Target compounds were prepared as in the following Table 4 in the same manner as in Preparation Example 2, except that Compound G of the following Table 4 was used instead of 2-bromoindolo[3,2,1-jk]carbazole, and Compound H of the following Table 4 was used instead of (4-(diphenylamino)phenyl)boronic acid. In addition, Compounds 1-151 and 1-157 in the following Table 4 were prepared in the same manner as in Preparation Example 2, except that 5-bromo-7-chloronaphtho[1,2-b]benzofuran substituted with deuterium was used instead of 5-bromo-7-chloronaphtho[1,2-b]benzofuran, Compound G of the following Table 4 was used instead of 2-bromoindolo[3,2,1-jk]carbazole, and Compound Hof the following Table 4 was used instead of (4-(diphenylamino)phenyl)boronic acid.

TABLE 4

| Com- | | | | |
|---|---|---|---|---|
| pound | | | | |
| No. | Compound G | Compound H | Target Compound | Yield |
| 1-19 | 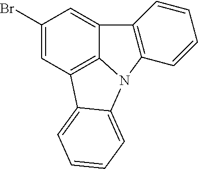 [SMILES: Brc1cc2c3ccccc3n3c4ccccc4c(c1)c23] | 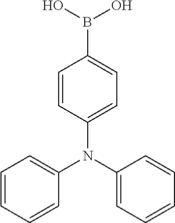 [SMILES: OB(O)c1ccc(N(c2ccccc2)c2ccccc2)cc1] | 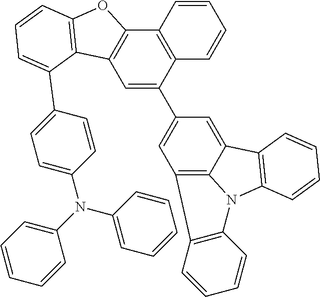 [SMILES: c1ccc(N(c2ccccc2)c2ccc(-c3cccc4oc5c6ccccc6c(-c6cc7c8ccccc8n8c9ccccc9c(c6)c78)cc5c34)cc2)cc1] | 75% |
| 1-20 | 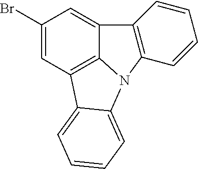 [SMILES: Brc1cc2c3ccccc3n3c4ccccc4c(c1)c23] | 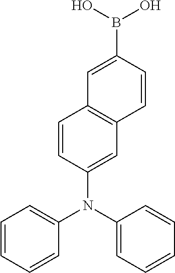 [SMILES: OB(O)c1ccc2cc(N(c3ccccc3)c3ccccc3)ccc2c1] | 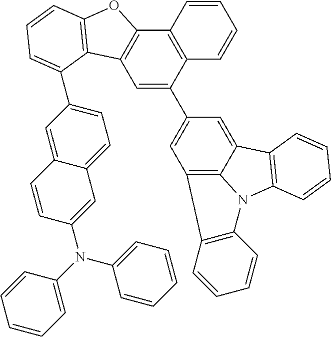 [SMILES: c1ccc(N(c2ccccc2)c2ccc3cc(-c4cccc5oc6c7ccccc7c(-c7cc8c9ccccc9n9c%10ccccc%10c(c7)c89)cc6c45)ccc3c2)cc1] | 79% |
| 1-21 | 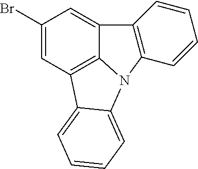 [SMILES: Brc1cc2c3ccccc3n3c4ccccc4c(c1)c23] | 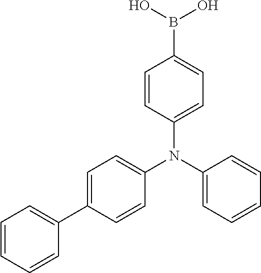 [SMILES: OB(O)c1ccc(N(c2ccccc2)c2ccc(-c3ccccc3)cc2)cc1] | 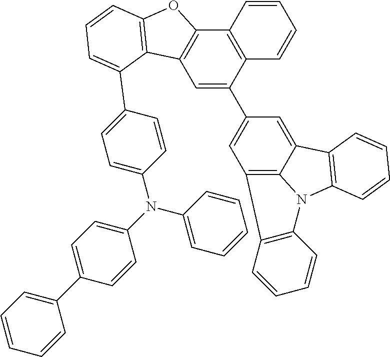 [SMILES: c1ccc(-c2ccc(N(c3ccccc3)c3ccc(-c4cccc5oc6c7ccccc7c(-c7cc8c9ccccc9n9c%10ccccc%10c(c7)c89)cc6c45)cc3)cc2)cc1] | 70% |
| 1-22 | 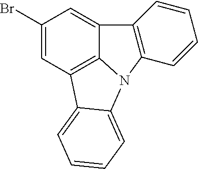 [SMILES: Brc1cc2c3ccccc3n3c4ccccc4c(c1)c23] | 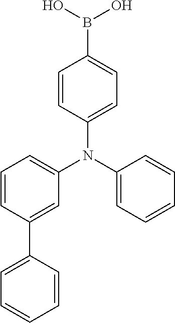 [SMILES: OB(O)c1ccc(N(c2ccccc2)c2cccc(-c3ccccc3)c2)cc1] | 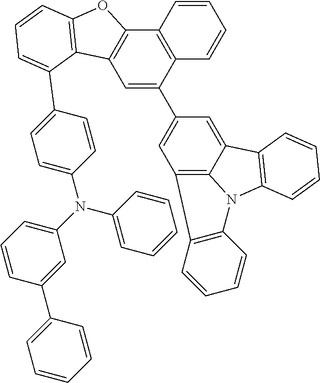 [SMILES: c1ccc(-c2cccc(N(c3ccccc3)c3ccc(-c4cccc5oc6c7ccccc7c(-c7cc8c9ccccc9n9c%10ccccc%10c(c7)c89)cc6c45)cc3)c2)cc1] | 81% |
| 1-23 | 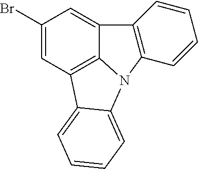 [SMILES: Brc1cc2c3ccccc3n3c4ccccc4c(c1)c23] | 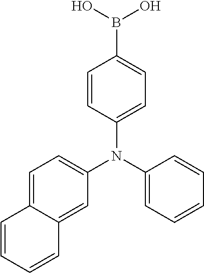 [SMILES: OB(O)c1ccc(N(c2ccccc2)c2ccc3ccccc3c2)cc1] | 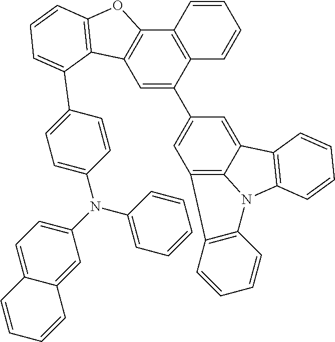 [SMILES: c1ccc(N(c2ccc(-c3cccc4oc5c6ccccc6c(-c6cc7c8ccccc8n8c9ccccc9c(c6)c78)cc5c34)cc2)c2ccc3ccccc3c2)cc1] | 81% |
| 1-44 | 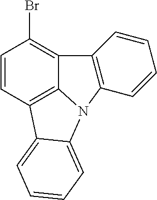 [SMILES: Brc1ccc2c3ccccc3n3c4ccccc4c1c23] | 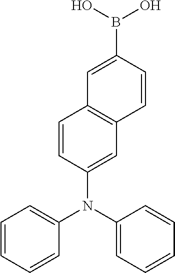 [SMILES: OB(O)c1ccc2cc(N(c3ccccc3)c3ccccc3)ccc2c1] | 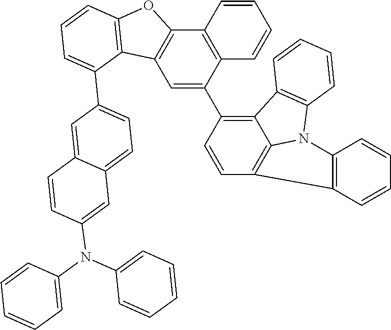 [SMILES: c1ccc(N(c2ccccc2)c2ccc3cc(-c4cccc5oc6c7ccccc7c(-c7ccc8c9ccccc9n9c%10ccccc%10c7c89)cc6c45)ccc3c2)cc1] | 77% |
| 1-45 | 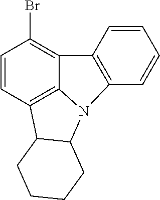 [SMILES: Brc1ccc2c3c1c1ccccc1n3C1CCCCC21] | 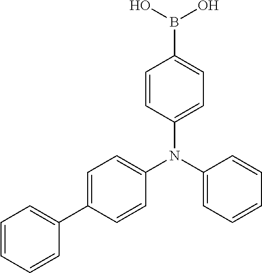 [SMILES: OB(O)c1ccc(N(c2ccccc2)c2ccc(-c3ccccc3)cc2)cc1] | 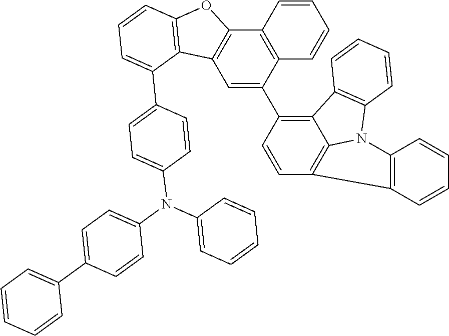 [SMILES: c1ccc(-c2ccc(N(c3ccccc3)c3ccc(-c4cccc5oc6c7ccccc7c(-c7ccc8c9ccccc9n9c%10ccccc%10c7c89)cc6c45)cc3)cc2)cc1] | 76% |
| 1-68 | 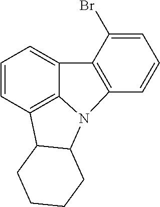 [SMILES: Brc1cccc2c1c1cccc3c1n2C1CCCCC31] | 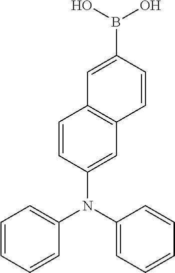 [SMILES: OB(O)c1ccc2cc(N(c3ccccc3)c3ccccc3)ccc2c1] | 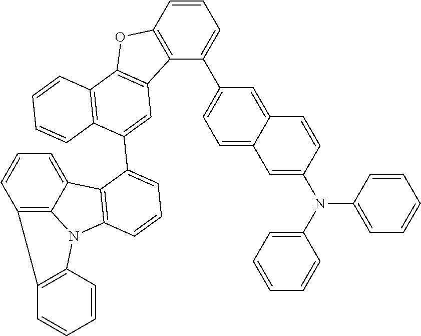 [SMILES: c1ccc(N(c2ccccc2)c2ccc3cc(-c4cccc5oc6c7ccccc7c(-c7cccc8c7c7cccc9c%10ccccc%10n8c97)cc6c45)ccc3c2)cc1] | 71% |
| 1-92 | 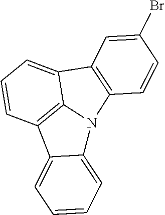 [SMILES: Brc1ccc2c(c1)c1cccc3c4ccccc4n2c31] | 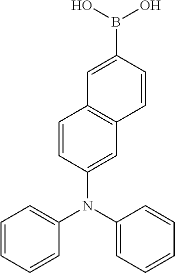 [SMILES: OB(O)c1ccc2cc(N(c3ccccc3)c3ccccc3)ccc2c1] | 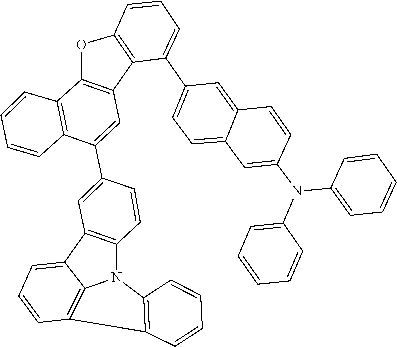 [SMILES: c1ccc(N(c2ccccc2)c2ccc3cc(-c4cccc5oc6c7ccccc7c(-c7ccc8c(c7)c7cccc9c%10ccccc%10n8c97)cc6c45)ccc3c2)cc1] | 74% |
| 1-94 | 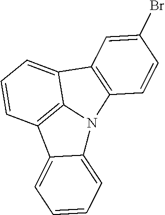 [SMILES: Brc1ccc2c(c1)c1cccc3c4ccccc4n2c31] | 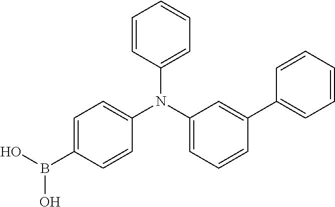 [SMILES: OB(O)c1ccc(N(c2ccccc2)c2cccc(-c3ccccc3)c2)cc1] | 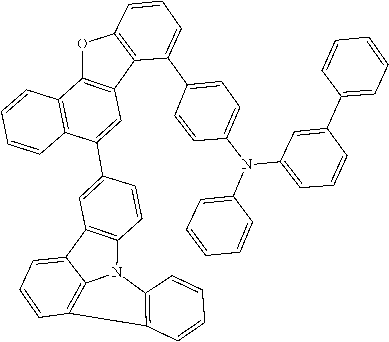 [SMILES: c1ccc(-c2cccc(N(c3ccccc3)c3ccc(-c4cccc5oc6c7ccccc7c(-c7ccc8c(c7)c7cccc9c%10ccccc%10n8c97)cc6c45)cc3)c2)cc1] | 81% |
| 1-120 | 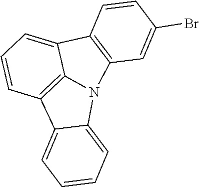 [SMILES: Brc1ccc2c3cccc4c5ccccc5n(c2c1)c43] / | 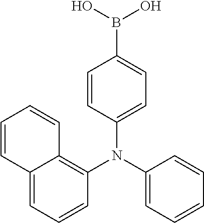 [SMILES: OB(O)c1ccc(N(c2ccccc2)c2cccc3ccccc23)cc1] | 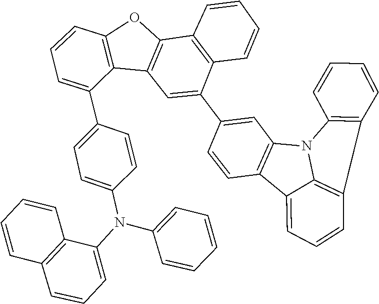 [SMILES: c1ccc(N(c2ccc(-c3cccc4oc5c6ccccc6c(-c6ccc7c8cccc9c%10ccccc%10n(c7c6)c98)cc5c34)cc2)c2cccc3ccccc23)cc1] | 76% |
| 1-142 | 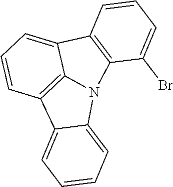 [SMILES: Brc1cccc2c3cccc4c5ccccc5n(c12)c43] | 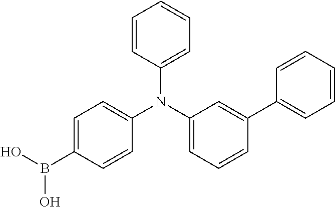 [SMILES: OB(O)c1ccc(N(c2ccccc2)c2cccc(-c3ccccc3)c2)cc1] | 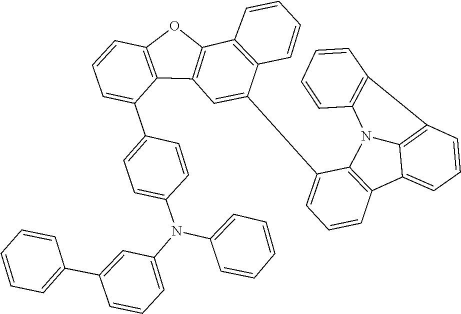 [SMILES: c1ccc(-c2cccc(N(c3ccccc3)c3ccc(-c4cccc5oc6c7ccccc7c(-c7cccc8c9cccc%10c%11ccccc%11n(c78)c%109)cc6c45)cc3)c2)cc1] | 85% |
| 1-151 | 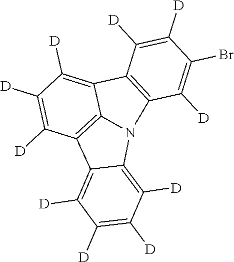 [SMILES: [2H]c1c([2H])c([2H])c2c(c1[2H])c1c([2H])c([2H])c([2H])c3c4c([2H])c([2H])c(Br)c([2H])c4n2c13] | 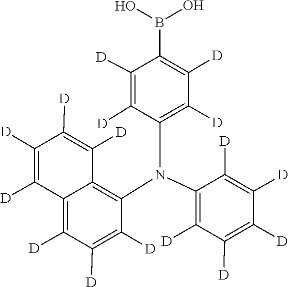 [SMILES: [2H]c1c([2H])c([2H])c(N(c2c([2H])c([2H])c(B(O)O)c([2H])c2[2H])c2c([2H])c([2H])c([2H])c3c([2H])c([2H])c([2H])c([2H])c23)c([2H])c1[2H]] | 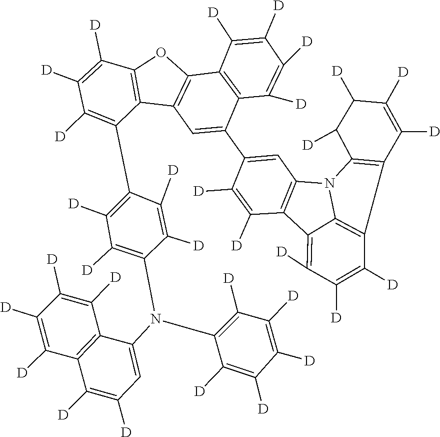 [SMILES: [2H]C1=C([2H])C([2H])C([2H])c2c1c1c([2H])c([2H])c([2H])c3c4c([2H])c([2H])c(-c5cc6c(oc7c([2H])c([2H])c([2H])c(-c8c([2H])c([2H])c(N(c9c([2H])c([2H])c([2H])c([2H])c9[2H])c9cc([2H])c([2H])c%10c([2H])c([2H])c([2H])c([2H])c9%10)c([2H])c8[2H])c76)c6c([2H])c([2H])c([2H])c([2H])c56)cc4n2c13] | 83% |
| 1-157 | 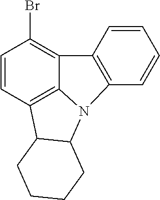 [SMILES: Brc1ccc2c3c1c1ccccc1n3C1CCCCC21] | 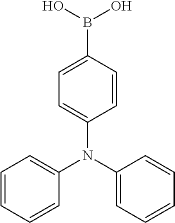 [SMILES: OB(O)c1ccc(N(c2ccccc2)c2ccccc2)cc1] | 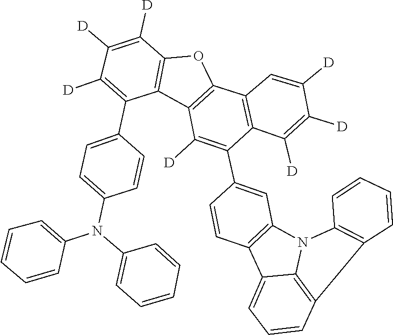 [SMILES: [2H]c1cc2c(c([2H])c1[2H])c(-c1ccc3c4cccc5c6ccccc6n(c3c1)c54)c([2H])c1c2oc2c([2H])c([2H])c([2H])c(-c3ccc(N(c4ccccc4)c4ccccc4)cc3)c21] | 79% |

Target compounds were prepared as in the following Table 5 in the same manner as in Preparation Example 2, except that 8-bromo-1-chloro-dibenzofuiran was used instead of 5-bromo-7-chloronaphtho[1,2-b]benzofuran, Compound I of the following Table 5 was used instead of 2-bromoindolo[3,2,1-jk]carbazole, and Compound J of the following Table 5 was used instead of (4-(diphenylamino)phenyl)boronic acid.

TABLE 5

| Com- | | | | |
|---|---|---|---|---|
| pound | | | | |
| No. | Compound I | Compound J | Target Compound | Yield |
| 2-22 | 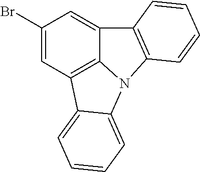 [SMILES: Brc1cc2c3ccccc3n3c4ccccc4c(c1)c23] | 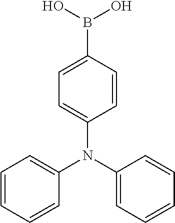 [SMILES: OB(O)c1ccc(N(c2ccccc2)c2ccccc2)cc1] | 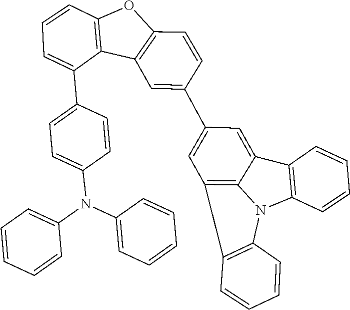 [SMILES: c1ccc(N(c2ccccc2)c2ccc(-c3cccc4oc5ccc(-c6cc7c8ccccc8n8c9ccccc9c(c6)c78)cc5c34)cc2)cc1] | 84% |
| 2-23 | 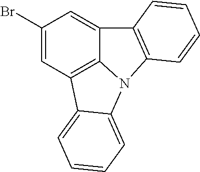 [SMILES: Brc1cc2c3ccccc3n3c4ccccc4c(c1)c23] | 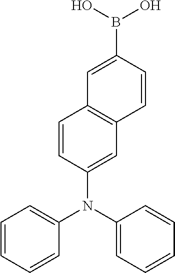 [SMILES: OB(O)c1ccc2cc(N(c3ccccc3)c3ccccc3)ccc2c1] | 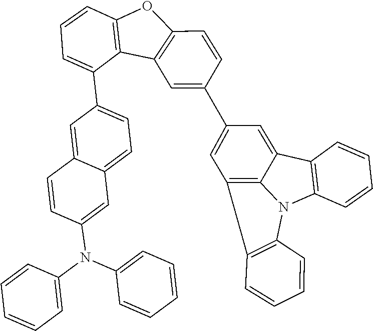 [SMILES: c1ccc(N(c2ccccc2)c2ccc3cc(-c4cccc5oc6ccc(-c7cc8c9ccccc9n9c%10ccccc%10c(c7)c89)cc6c45)ccc3c2)cc1] | 81% |
| 2-25 | 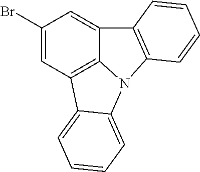 [SMILES: Brc1cc2c3ccccc3n3c4ccccc4c(c1)c23] | 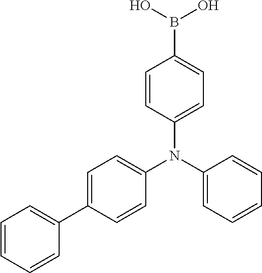 [SMILES: OB(O)c1ccc(N(c2ccccc2)c2ccc(-c3ccccc3)cc2)cc1] | 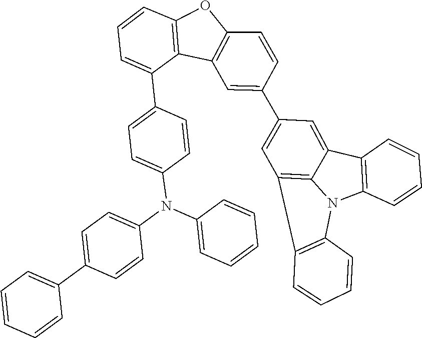 [SMILES: c1ccc(-c2ccc(N(c3ccccc3)c3ccc(-c4cccc5oc6ccc(-c7cc8c9ccccc9n9c%10ccccc%10c(c7)c89)cc6c45)cc3)cc2)cc1] | 79% |
| 2-28 | 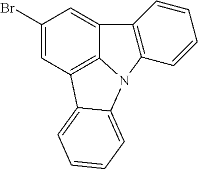 [SMILES: Brc1cc2c3ccccc3n3c4ccccc4c(c1)c23] | 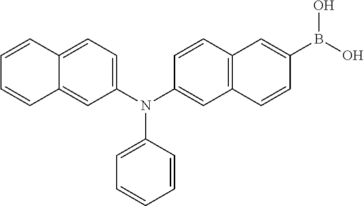 [SMILES: OB(O)c1ccc2cc(N(c3ccccc3)c3ccc4ccccc4c3)ccc2c1] | 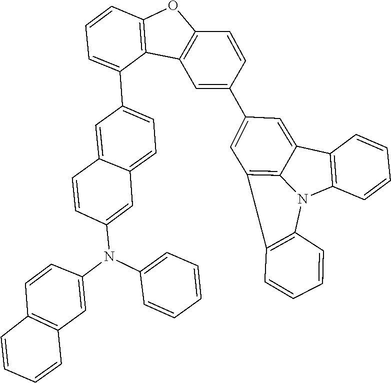 [SMILES: c1ccc(N(c2ccc3ccccc3c2)c2ccc3cc(-c4cccc5oc6ccc(-c7cc8c9ccccc9n9c%10ccccc%10c(c7)c89)cc6c45)ccc3c2)cc1] | 79% |
| 2-52 | 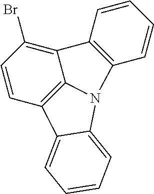 [SMILES: Brc1ccc2c3ccccc3n3c4ccccc4c1c23] | 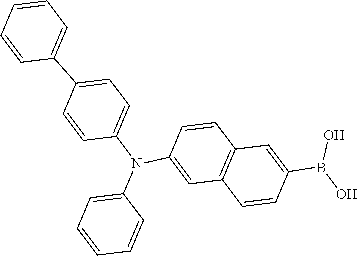 [SMILES: OB(O)c1ccc2cc(N(c3ccccc3)c3ccc(-c4ccccc4)cc3)ccc2c1] | 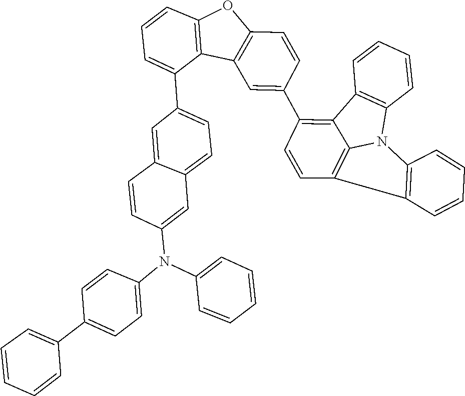 [SMILES: c1ccc(-c2ccc(N(c3ccccc3)c3ccc4cc(-c5cccc6oc7ccc(-c8ccc9c%10ccccc%10n%10c%11ccccc%11c8c9%10)cc7c56)ccc4c3)cc2)cc1] | 83% |
| 2-134 | 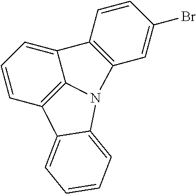 [SMILES: Brc1ccc2c3cccc4c5ccccc5n(c2c1)c43] | 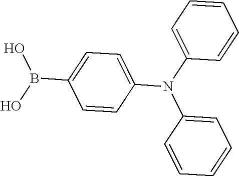 [SMILES: OB(O)c1ccc(N(c2ccccc2)c2ccccc2)cc1] | 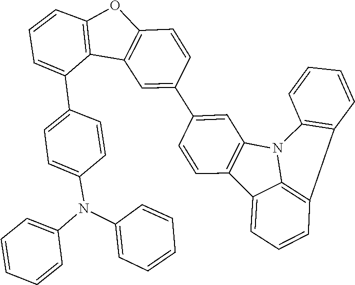 [SMILES: c1ccc(N(c2ccccc2)c2ccc(-c3cccc4oc5ccc(-c6ccc7c8cccc9c%10ccccc%10n(c7c6)c98)cc5c34)cc2)cc1] | 84% |
| 2-139 | 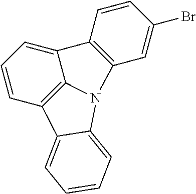 [SMILES: Brc1ccc2c3cccc4c5ccccc5n(c2c1)c43] | 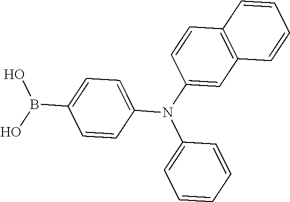 [SMILES: OB(O)c1ccc(N(c2ccccc2)c2ccc3ccccc3c2)cc1] | 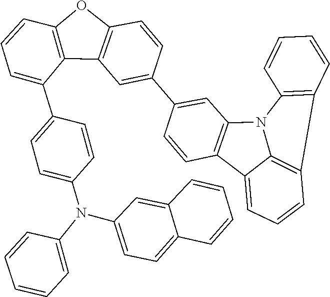 [SMILES: c1ccc(N(c2ccc(-c3cccc4oc5ccc(-c6ccc7c8cccc9c%10ccccc%10n(c7c6)c98)cc5c34)cc2)c2ccc3ccccc3c2)cc1] | 81% |

Target compounds were prepared as in the following Table 6 in the same manner as in Preparation Example 2, except that 1-bromo-8-chloro-dibenzofuiran was used instead of 5-bromo-7-chloronaphtho[1,2-b]benzofuran, Compound K of the following Table 6 was used instead of 2-bromoindolo[3,2,1-jk]carbazole, and Compound L of the following Table 6 was used instead of (4-(diphenylamino)phenyl)boronic acid.

TABLE 6

| Com- | | | | |
|---|---|---|---|---|
| pound | | | | |
| No. | Compound K | Compound L | Target Compound | Yield |
| 2-78 | 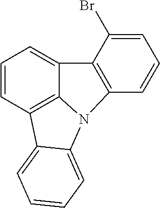 [SMILES: Brc1cccc2c1c1cccc3c4ccccc4n2c31] | 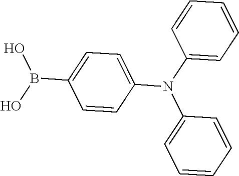 [SMILES: OB(O)c1ccc(N(c2ccccc2)c2ccccc2)cc1] | 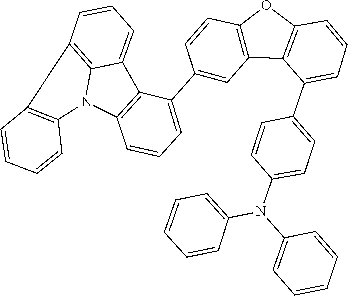 [SMILES: c1ccc(N(c2ccccc2)c2ccc(-c3cccc4oc5ccc(-c6cccc7c6c6cccc8c9ccccc9n7c86)cc5c34)cc2)cc1] | 70% |
| 2-108 | 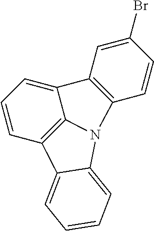 [SMILES: Brc1ccc2c(c1)c1cccc3c4ccccc4n2c31] | 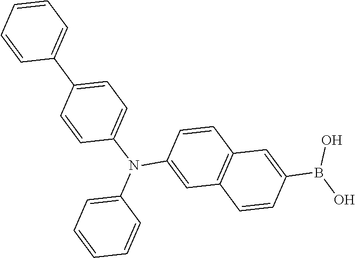 [SMILES: OB(O)c1ccc2cc(N(c3ccccc3)c3ccc(-c4ccccc4)cc3)ccc2c1] | 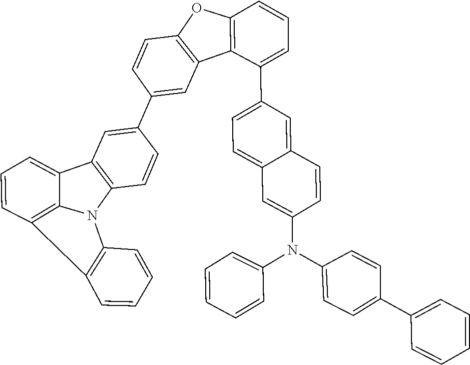 [SMILES: c1ccc(-c2ccc(N(c3ccccc3)c3ccc4cc(-c5cccc6oc7ccc(-c8ccc9c(c8)c8cccc%10c%11ccccc%11n9c%108)cc7c56)ccc4c3)cc2)cc1] | 80% |
| 2-109 | 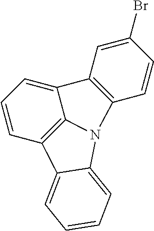 [SMILES: Brc1ccc2c(c1)c1cccc3c4ccccc4n2c31] | 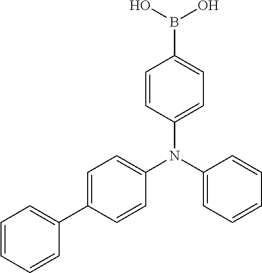 [SMILES: OB(O)c1ccc(N(c2ccccc2)c2ccc(-c3ccccc3)cc2)cc1] | 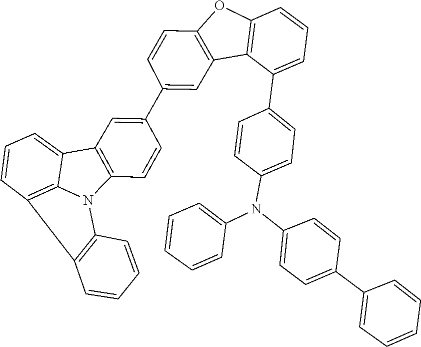 [SMILES: c1ccc(-c2ccc(N(c3ccccc3)c3ccc(-c4cccc5oc6ccc(-c7ccc8c(c7)c7cccc9c%10ccccc%10n8c97)cc6c45)cc3)cc2)cc1] | 80% |
| 2-178 | 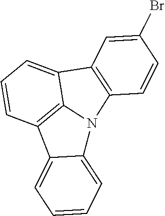 [SMILES: Brc1ccc2c(c1)c1cccc3c4ccccc4n2c31] | 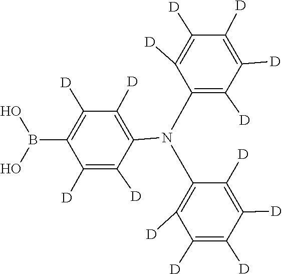 [SMILES: [2H]c1c([2H])c([2H])c(N(c2c([2H])c([2H])c([2H])c([2H])c2[2H])c2c([2H])c([2H])c(B(O)O)c([2H])c2[2H])c([2H])c1[2H]] | 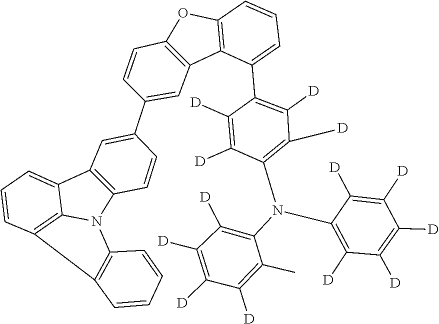 [SMILES: [2H]c1c([2H])c([2H])c(N(c2c([2H])c([2H])c(-c3cccc4oc5ccc(-c6ccc7c(c6)c6cccc8c9ccccc9n7c86)cc5c34)c([2H])c2[2H])c2c([2H])c([2H])c([2H])c([2H])c2C)c([2H])c1[2H]] | 76% |

The rest of compounds other than the compounds described in Preparation Examples 1 and 2 and Tables 1 to 6 were also prepared in the same maimer as the methods described in the Preparation Examples described above, and the synthesis results are shown in the following Tables 7 and 8. The following Table 7 shows measurement values of ¹H NMR (CDCl₃, 400 MHz), and the following Table 8 shows measurement values of field desorption mass spectrometry (FD-MS).

TABLE 7

| Compound | |
|---|---|
| No. | ¹H NMR (CDCl₃, 400 MHz) |
| 1-1 | δ = 8.97(1H, s), 8.19(2H, d), 8.18(1H, d), 7.65(2H, d), 7.64(1H, d), 7.59(1H, d), 7.58(2H, d), |
| | 7.5(2H, d), 7.38(1H, d), 7.34(1H, dd), 7.25(1H, d), 7.24(4H, d), 7.2(2H, dd), 7.08(4H, dd), |
| | 7(2H, s), 6.91 (1H, t), |
| 1-2 | δ = 8.97(1H, s), 8.19(2H, d), 8.18(1H, d), 7.75(2H, d), 7.65(2H, d), 7.64(1H, d), 7.59(1H, d), |
| | 7.58(2H, d), 7.55(2H, d), 7.5(2H, dd), 7.49(2H, d), 7.38(1H, d), 7.37(2H, dd), 7.34(1H, dd), |
| | 7.25(1H, s), 7.24 (2H, t), 7.2 (2H, t), 7.08 (2H, dd), 6.91 (1H, dd), |
| 1-3 | δ = 8.97(1H, s), 8.19(2H, d), 8.18(1H, d), 7.75(2H, d), 7.65(2H, d), 7.64(1H, d), 7.58(2H, d), |
| | 7.55(2H, d), 7.5(2H, d), 7.38(1H, dd), 7.37(2H, d), 7.34(1H, d), 7.25(5H, dd), 7.24(2H, dd), |
| | 7.2(2H, s), 7.08 (2H, t), 6.91 (1H, t), |
| 1-4 | δ = 8.97(1H, s), 8.19(2H, d), 8.18(1H, d), 7.75(4H, d), 7.65(2H, d), 7.64(1H, d), 7.59(1H, d), |
| | 7.58(2H, d), 7.55(4H, d), 7.5(2H, dd), 7.38(1H, d), 7.37(4H, d), 7.34(1H, dd), 7.25(1H, dd), |
| | 7.2(2H, s), 6.91 (1H, t), |
| 1-5 | δ = 8.97(1H, s), 8.19(2H, d), 8.18(1H, d), 7.75(2H, d), 7.65(2H, d), 7.64(1H, d), 7.59(1H, d), |
| | 7.58(2H, d), 7.55(1H, d), 7.5(2H, dd), 7.49(2H, d), 7.38(1H, d), 7.34(1H, dd), 7.27(1H, dd), |
| | 7.25(1H, s), 7.24 (2H, t), 7.2 (2H, t), 7.18 (1H, dd), 7.17 (1H, dd), 7.08 (2H, t), 6.91 (1H, d), |
| 1-6 | δ = 8.97(1H, s), 8.19(2H, d), 8.18(1H, d), 8.09(1H, d), 8.06(1H, d), 7.99(1H, d), 7.65(2H, d), |
| | 7.64(1H, d), 7.59(1H, d), 7.58(2H, dd), 7.55(3H, d), 7.5(2H, d), 7.38(2H, dd), 7.37(2H, dd), |
| | 7.34(1H, s), 7.25 (1H, t), 7.24 (2H, t), 7.2 (2H, dd), 7.08 (2H, dd), 6.91 (1H, t), |
| 1-7 | δ = 8.97(1H, s), 8.19(2H, d), 8.18(1H, d), 7.75(4H, d), 7.65(2H, d), 7.64(1H, d), 7.59(1H, d), |
| | 7.58(2H, d), 7.55(3H, d), 7.5(2H, dd), 7.38(1H, d), 7.37(2H, d), 7.34(1H, dd), 7.27(1H, dd), |
| | 7.25(1H, s), 7.2 (2H, t), 7.18 (1H, t), 7.17 (1H, dd), 6.91 (1H, dd), |
| 1-8 | δ = 8.97(1H, s), 8.19(2H, d), 8.18(1H, d), 8.1(1H, d), 7.75(2H, d), 7.65(2H, d), 7.64(1H, d), |
| | 7.59(1H, d), 7.58(2H, d), 7.55(2H, dd), 7.5(2H, d), 7.39(1H, d), 7.38(1H, dd), 7.37(3H, dd), |
| | 7.34(1H, s), 7.25 (1H, t), 7.2 (2H, t), 7.14 (1H, dd), 7.08 (2H, dd), 6.91 (1H, t), |
| 1-9 | δ = 8.97(1H, s), 8.19(2H, d), 8.18(1H, d), 7.84(1H, d), 7.75(2H, d), 7.65(2H, d), 7.64(1H, d), |
| | 7.59(1H, d), 7.58(2H, d), 7.56(1H, dd), 7.5(2H, d), 7.49(2H, d), 7.4(1H, dd), 7.38(1H, dd), |
| | 7.34(1H, s), 7.32 (1H, t), 7.25 (1H, t), 7.24 (2H, dd), 7.2 (2H, dd), 7.17 (1H, t), 7.11 (1H, d), |
| | 7.08 (2H, d), 6.91 (1H, d), |
| 1-10 | δ = 8.97(1H, s), 8.95(1H, d), 8.5(1H, d), 8.2(1H, d), 8.19(2H, d), 8.18(1H, d), 8.09(1H, d), |
| | 7.77(1H, d), 7.65(2H, d), 7.64(1H, dd), 7.59(1H, d), 7.58(2H, d), 7.55(2H, dd), 7.5(2H, dd), |
| | 7.38(1H, s), 7.37 (2H, t), 7.34 (1H, t), 7.25 (1H, dd), 7.24 (2H, dd), 7.2 (2H, t), 7.08 (2H, d), |
| | 6.91 (1H, d), |
| 1-11 | δ = 8.97(1H, s), 8.19(2H, d), 8.18(1H, d), 7.94(1H, d), 7.75(2H, d), 7.73(1H, d), 7.65(2H, d), |
| | 7.64(1H, d), 7.61(2H, d), 7.58(2H, dd), 7.55(2H, d), 7.5(2H, d), 7.38(1H, dd), 7.37(2H, dd), |
| | 7.34(1H, s), 7.25 (1H, t), 7.24 (2H, t), 7.2 (2H, dd), 7.08 (2H, dd), 6.91 (1H, t), |
| 1-12 | δ = 8.97(1H, s), 8.19(2H, d), 8.18(1H, d), 7.98(1H, d), 7.65(2H, d), 7.64(1H, d), 7.59(1H, d), |
| | 7.58(2H, d), 7.54(1H, d), 7.5(2H, dd), 7.39(1H, d), 7.38(1H, d), 7.34(2H, dd), 7.31(1H, dd), |
| | 7.25(2H, s), 7.24 (2H, t), 7.2 (2H, t), 7.08 (2H, dd), 7 (1H, dd), 6.91 (2H, t), |
| 1-13 | δ = 8.97(1H, s), 8.22(1H, d), 8.19(2H, d), 8.18(1H, d), 7.98(1H, d), 7.65(2H, d), 7.64(1H, d), |
| | 7.59(1H, d), 7.58(2H, d), 7.56(1H, dd), 7.54(1H, d), 7.5(2H, d), 7.39(1H, dd), 7.38(1H, dd), |
| | 7.34(1H, s), 7.31 (1H, t), 7.25 (1H, t), 7.24 (2H, dd), 7.2 (2H, dd), 7.08 (2H, t), 7 (1H, d), |
| | 6.97 (1H, d), 6.91 (1H, d), |
| 1-14 | δ = 8.97(1H, s), 8.19(2H, d), 8.18(1H, d), 8.03(1H, d), 7.98(1H, d), 7.8(1H, d), 7.65(2H, d), |
| | 7.64(1H, d), 7.59(1H, d), 7.58(2H, dd), 7.54(1H, d), 7.5(2H, d), 7.39(1H, dd), 7.38(1H, dd), |
| | 7.34(1H, s), 7.31 (1H, t), 7.25 (1H, t), 7.24 (2H, dd), 7.2 (2H, dd), 7.08 (2H, t), 7 (1H, d), |
| | 6.91 (2H, d), |
| 1-15 | δ = 8.97(1H, s), 8.19(3H, d), 8.18(1H, d), 8.01(1H, d), 7.9(1H, d), 7.65(2H, d), 7.64(1H, d), |
| | 7.62(2H, d), 7.58(4H, d), 7.5(5H, dd), 7.38(1H, d), 7.34(1H, d), 7.25(1H, dd), 7.24(2H, dd), |
| | 7.2(3H, s), 7.08 (2H, t), 6.91 (1H, t), 6.48 (1H, dd), |
| 1-16 | δ = 8.97(1H, s), 8.55(1H, d), 8.24(1H, d), 8.19(2H, d), 8.18(1H, d), 7.94(1H, d), 7.65(2H, d), |
| | 7.64(1H, d), 7.62(2H, d), 7.58(3H, dd), 7.5(4H, d), 7.38(1H, d), 7.35(1H, dd), 7.34(1H, dd), |
| | 7.27(1H, s), 7.25 (2H, t), 7.24 (2H, t), 7.2 (2H, dd), 7.16 (1H, dd), 7.08 (2H, t), 6.91 (1H, d), |
| 1-17 | δ = 8.97(1H, s), 8.45(1H, d), 8.19(2H, d), 8.18(1H, d), 7.95(1H, d), 7.93(1H, d), 7.85(1H, d), |
| | 7.65(2H, d), 7.64(1H, d), 7.59(1H, dd), 7.58(2H, d), 7.56(1H, d), 7.5(2H, dd), 7.49(1H, dd), |
| | 7.41(1H, s), 7.38 (1H, t), 7.34 (1H, t), 7.25 (1H, dd), 7.24 (2H, dd), 7.2 (2H, t), 7.08 (2H, d), |
| | 7 (1H, d), 6.91 (1H, d), |
| 1-18 | δ = 8.97(1H, s), 8.45(1H, d), 8.19(2H, d), 8.18(1H, d), 8.01(1H, d), 7.93(1H, d), 7.65(2H, d), |
| | 7.64(2H, d), 7.59(1H, d), 7.58(2H, dd), 7.56(1H, d), 7.5(2H, d), 7.49(1H, dd), 7.43(1H, dd), |
| | 7.38(1H, s), 7.34 (1H, t), 7.25 (1H, t), 7.24 (2H, dd), 7.2 (2H, dd), 7.08 (2H, t), 7 (1H, d), |
| | 6.91 (1H, d), |
| 1-19 | δ = 8.97(1H, s), 8.19(2H, d), 8.18(1H, d), 7.82(1H, d), 7.69(1H, d), 7.65(2H, d), 7.64(1H, d), |
| | 7.59(1H, d), 7.58(2H, d), 7.57(1H, dd), 7.55(2H, d), 7.5(2H, d), 7.38(1H, dd), 7.37(2H, dd), |
| | 7.24(4H, s), 7.2 (2H, t), 7.08 (4H, t), |
| 1-20 | δ = 8.97(1H, s), 8.19(2H, d), 8.18(1H, d), 7.84(1H, d), 7.82(1H, d), 7.69(1H, d), 7.65(2H, d), |
| | 7.64(1H, d), 7.59(1H, d), 7.58(2H, dd), 7.57(1H, d), 7.56(1H, d), 7.5(2H, dd), 7.4(1H, dd), |
| | 7.38(1H, s), 7.32 (1H, t), 7.24 (4H, t), 7.2 (2H, dd), 7.17 (1H, dd), 7.11 (1H, t), 7.08 (4H, d), |
| 1-21 | δ = 8.97(1H, s), 8.19(2H, d), 8.18(1H, d), 7.82(1H, d), 7.75(2H, d), 7.69(1H, d), 7.65(2H, d), |
| | 7.64(1H, d), 7.58(2H, d), 7.57(1H, dd), 7.55(4H, d), 7.5(2H, d), 7.38(1H, dd), 7.37(4H, dd), |
| | 7.24(2H, s), 7.2 (2H, t), 7.08 (2H, t), |
| 1-22 | δ = 8.97(1H, s), 8.19(2H, d), 8.18(1H, d), 7.82(1H, d), 7.75(2H, d), 7.69(1H, d), 7.65(2H, d), |
| | 7.64(1H, d), 7.58(2H, d), 7.57(1H, dd), 7.55(3H, d), 7.5(2H, d), 7.38(1H, dd), 7.37(2H, dd), |
| | 7.27(1H, s), 7.24 (2H, t), 7.2 (2H, t), 7.18 (1H, dd), 7.17 (1H, dd), 7.08 (2H, t), |
| 1-23 | δ = 8.97(1H, s), 8.19(2H, d), 8.18(1H, d), 7.82(1H, d), 7.78(1H, d), 7.71(1H, d), 7.69(1H, d), |
| | 7.65(2H, d), 7.64(1H, d), 7.59(1H, dd), 7.58(2H, d), 7.57(1H, d), 7.55(2H, dd), 7.54(1H, dd), |
| | 7.5(2H, s), 7.42 (1H, t), 7.38 (1H, t), 7.37 (2H, dd), 7.24 (2H, dd), 7.2 (2H, t), 7.11 |
| | (1H, d), 7.08 (2H, d), |
| 1-25 | δ = 8.97(1H, s), 8.19(2H, d), 8.18(1H, d), 7.91(1H, d), 7.84(1H, d), 7.64(1H, d), 7.59(1H, d), |
| | 7.58(2H, d), 7.5(2H, d), 7.38(1H, dd), 7.34(1H, d), 7.25(1H, d), 7.24(4H, dd), 7.2(2H, dd), |
| | 7.08(4H, s), 7 (2H, t), 6.91 (1H, t), |
| 1-28 | δ = 8.97(1H, s), 8.19(2H, d), 8.18(1H, d), 7.91(1H, d), 7.84(1H, d), 7.75(4H, d), 7.64(1H, d), |
| | 7.59(1H, d), 7.58(2H, d), 7.55(4H, dd), 7.5(2H, d), 7.38(1H, d), 7.37(4H, dd), 7.34(1H, dd), |
| | 7.25(1H, s), 7.2 (2H, t), 6.91 (1H, t), |
| 1-30 | δ = 8.97(1H, s), 8.19(2H, d), 8.18(1H, d), 8.09(1H, d), 8.06(1H, d), 7.99(1H, d), 7.91(1H, d), |
| | 7.84(1H, d), 7.64(1H, d), 7.59(1H, dd), 7.58(2H, d), 7.55(3H, d), 7.5(2H, dd), 7.38(2H, dd), |
| | 7.37(2H, s), 7.34 (1H, t), 7.25 (1H, t), 7.24 (2H, dd), 7.2 (2H, dd), 7.08 (2H, t), 6.91 (1H, d), |
| 1-37 | δ = 8.97(1H, s), 8.22(1H, d), 8.19(2H, d), 8.18(1H, d), 7.98(1H, d), 7.91(1H, d), 7.84(1H, d), |
| | 7.64(1H, d), 7.59(1H, d), 7.58(2H, dd), 7.56(1H, d), 7.54(1H, d), 7.5(2H, dd), 7.39(1H, dd), |
| | 7.38(1H, s), 7.34 (1H, t), 7.31 (1H, t), 7.25 (1H, dd), 7.24 (2H, dd), 7.2 (2H, t), 7.08 (2H, d), |
| | 7 (1H, d), 6.97 (1H, d), |
| 1-40 | δ = 8.97(1H, s), 8.55(1H, d), 8.24(1H, d), 8.19(2H, d), 8.18(1H, d), 7.94(1H, d), 7.91(1H, d), |
| | 7.84(1H, d), 7.64(1H, d), 7.62(2H, dd), 7.58(3H, d), 7.5(4H, d), 7.38(1H, dd), 7.35(1H, dd), |
| | 7.34(1H, s), 7.27 (1H, t), 7.25 (2H, t), 7.24 (2H, dd), 7.2 (2H, dd), 7.16 (1H, t), 7.08 (2H, d), |
| | 6.91 (1H, d), |
| 1-44 | δ = 8.97(1H, s), 8.19(2H, d), 8.18(1H, d), 7.91(1H, d), 7.84(2H, d), 7.82(1H, d), 7.69(1H, d), |
| | 7.64(1H, d), 7.59(1H, d), 7.58(2H, dd), 7.57(1H, d), 7.56(1H, d), 7.5(2H, dd), 7.4(1H, dd), |
| | 7.38(1H, s), 7.32 (1H, t), 7.24 (4H, t), 7.2 (2H, dd), 7.17 (1H, dd), 7.11 (1H, t), 7.08 (4H, d), |
| 1-45 | δ = 8.97(1H, s), 8.19(2H, d), 8.18(1H, d), 7.91(1H, d), 7.84(1H, d), 7.82(1H, d), 7.75(2H, d), |
| | 7.69(1H, d), 7.64(1H, d), 7.58(2H, dd), 7.57(1H, d), 7.55(4H, d), 7.5(2H, dd), 7.38(1H, dd), |
| | 7.37(4H, s), 7.24 (2H, t), 7.2 (2H, t), 7.08 (2H, dd), |
| 1-49 | δ = 8.97(1H, s), 8.19(2H, d), 8.18(1H, d), 7.91(1H, d), 7.72(1H, d), 7.68(1H, d), 7.64(1H, d), |
| | 7.59(1H, d), 7.58(1H, d), 7.56(1H, dd), 7.5(1H, d), 7.38(1H, d), 7.34(1H, dd), 7.29(1H, dd), |
| | 7.25(1H, s), 7.24 (4H, t), 7.2 (1H, t), 7.08 (4H, dd), 7 (2H, dd), 6.91 (1H, t), |
| 1-51 | δ = 8.97(1H, s), 8.19(2H, d), 8.18(1H, d), 7.91(1H, d), 7.75(2H, d), 7.72(1H, d), 7.68(1H, d), |
| | 7.64(1H, d), 7.58(1H, d), 7.56(1H, dd), 7.55(2H, d), 7.5(1H, d), 7.38(1H, dd), 7.37(2H, dd), |
| | 7.34(1H, s), 7.29 (1H, t), 7.25 (5H, t), 7.24 (2H, dd), 7.2 (1H, dd), 7.08 (2H, t), 6.91 (1H, d), |
| 1-55 | δ = 8.97(1H, s), 8.19(2H, d), 8.18(1H, d), 7.91(1H, d), 7.75(4H, d), 7.72(1H, d), 7.68(1H, d), |
| | 7.64(1H, d), 7.59(1H, d), 7.58(1H, dd), 7.56(1H, d), 7.55(3H, d), 7.5(1H, dd), 7.38(1H, dd), |
| | 7.37(2H, s), 7.34 (1H, t), 7.29 (1H, t), 7.27 (1H, dd), 7.25 (1H, dd), 7.2 (1H, t), 7.18 (1H, d), |
| | 7.17 (1H, d), 6.91 (1H, d), |
| 1-59 | δ = 8.97(1H, s), 8.19(2H, d), 8.18(1H, d), 7.94(1H, d), 7.91(1H, d), 7.75(2H, d), 7.73(1H, d), |
| | 7.72(1H, d), 7.68(1H, d), 7.64(1H, dd), 7.61(2H, d), 7.58(1H, d), 7.56(1H, dd), 7.55(2H, dd), |
| | 7.5(1H, s), 7.38 (1H, t), 7.37 (2H, t), 7.34 (1H, dd), 7.29 (1H, dd), 7.25 (1H, t), 7.24 |
| | (2H, d), 7.2 (1H, d), 7.08 (2H, d), |
| 1-65 | δ = 8.97(1H, s), 8.45(1H, d), 8.19(2H, d), 8.18(1H, d), 7.95(1H, d), 7.93(1H, d), 7.91(1H, d), |
| | 7.85(1H, d), 7.72(1H, d), 7.68(1H, dd), 7.64(1H, d), 7.59(1H, d), 7.58(1H, dd), |
| | 7.56(2H, dd), 7.5(1H, s), 7.49 (1H, t), 7.41 (1H, t), 7.38 (1H, dd), 7.34 (1H, dd), 7.29 (1H, t), 7.25 |
| | (1H, d), 7.24 (2H, d), 7.2 (1H, d), |
| 1-68 | δ = 8.97(1H, s), 8.19(2H, d), 8.18(1H, d), 7.91(1H, d), 7.84(1H, d), 7.82(1H, d), 7.72(1H, d), |
| | 7.69(1H, d), 7.68(1H, d), 7.64(1H, dd), 7.59(1H, d), 7.58(1H, d), 7.57(1H, dd), 7.56(2H, dd), |
| | 7.5(1H, s), 7.4 (1H, t), 7.38 (1H, t), 7.32 (1H, dd), 7.29 (1H, dd), 7.24 (4H, t), 7.2 |
| | (1H, d), 7.17 (1H, d), 7.11 (1H, d), |
| 1-73 | δ = 8.97(1H, s), 8.19(2H, d), 8.18(1H, d), 7.99(1H, d), 7.89(1H, d), 7.77(1H, d), 7.72(1H, d), |
| | 7.64(1H, d), 7.59(1H, d), 7.58(1H, dd), 7.5(1H, d), 7.38(1H, d), 7.34(1H, dd), 7.29(1H, dd), |
| | 7.25(1H, s), 7.24 (4H, t), 7.2 (1H, t), 7.08 (4H, dd), 7 (2H, dd), 6.91 (1H, t), |
| 1-78 | δ = 8.97(1H, s), 8.19(2H, d), 8.18(1H, d), 8.09(1H, d), 8.06(1H, d), 7.99(2H, d), 7.89(1H, d), |
| | 7.77(1H, d), 7.72(1H, d), 7.64(1H, dd), 7.59(1H, d), 7.58(1H, d), 7.55(3H, dd), 7.5(1H, dd), |
| | 7.38(2H, s), 7.37 (2H, t), 7.34 (1H, t), 7.29 (1H, dd), 7.25 (1H, dd), 7.24 (2H, t), 7.2 (1H, d), |
| | 7.08 (2H, d), 6.91 (1H, d), |
| 1-88 | δ = 8.97(1H, s), 8.55(1H, d), 8.24(1H, d), 8.19(2H, d), 8.18(1H, d), 7.99(1H, d), 7.94(1H, d), |
| | 7.89(1H, d), 7.77(1H, d), 7.72(1H, dd), 7.64(1H, d), 7.62(2H, d), 7.58(2H, dd), 7.5(3H, dd), |
| | 7.38(1H, s), 7.35 (1H, t), 7.34 (1H, t), 7.29 (1H, dd), 7.27 (1H, dd), 7.25 (2H, t), 7.24 |
| | (2H, d), 7.2 (1H, d), 7.16 (1H, d), |
| 1-92 | δ = 8.97(1H, s), 8.19(2H, d), 8.18(1H, d), 7.99(1H, d), 7.89(1H, d), 7.84(1H, d), 7.82(1H, d), |
| | 7.77(1H, d), 7.72(1H, d), 7.69(1H, dd), 7.64(1H, d), 7.59(1H, d), 7.58(1H, dd), 7.57(1H, |
| | dd), 7.56(1H, s), 7.5 (1H, t), 7.4 (1H, t), 7.38 (1H, dd), 7.32 (1H, dd), 7.29 (1H, t), 7.24 |
| | (4H, d), 7.2 (1H, d), 7.17 (1H, d), |
| 1-94 | δ = 8.97(1H, s), 8.19(2H, d), 8.18(1H, d), 7.99(1H, d), 7.89(1H, d), 7.82(1H, d), 7.77(1H, d), |
| | 7.75(2H, d), 7.72(1H, d), 7.69(1H, dd), 7.64(1H, d), 7.58(1H, d), 7.57(1H, dd), 7.55(3H, |
| | dd), 7.5(1H, s), 7.38 (1H, t), 7.37 (2H, t), 7.29 (1H, dd), 7.27 (1H, dd), 7.24 (2H, t), 7.2 |
| | (1H, d), 7.18 (1H, d), 7.17 (1H, d), |
| 1-97 | δ = 8.97(1H, s), 8.31(1H, d), 8.19(2H, d), 8.18(1H, d), 7.91(1H, d), 7.74(1H, d), 7.72(1H, d), |
| | 7.64(1H, d), 7.59(1H, d), 7.58(1H, dd), 7.5(1H, d), 7.38(1H, d), 7.34(1H, dd), 7.29(1H, dd), |
| | 7.25(1H, s), 7.24 (4H, t), 7.2 (1H, t), 7.08 (4H, dd), 7 (2H, dd), 6.91 (1H, t), |
| 1-100 | δ = 8.97(1H, s), 8.31(1H, d), 8.19(2H, d), 8.18(1H, d), 7.91(1H, d), 7.75(4H, d), 7.74(1H, d), |
| | 7.72(1H, d), 7.64(1H, d), 7.59(1H, dd), 7.58(1H, d), 7.55(3H, d), 7.5(1H, dd), 7.38(1H, dd), |
| | 7.37(2H, s), 7.34 (1H, t), 7.29 (1H, t), 7.27 (1H, dd), 7.25 (1H, dd), 7.2 (1H, t), 7.18 (1H, d), |
| | 7.17 (1H, d), 6.91 (1H, d), |
| 1-106 | δ = 8.97(1H, s), 8.95(1H, d), 8.5(1H, d), 8.31(1H, d), 8.2(1H, d), 8.19(2H, d), 8.18(1H, d), |
| | 8.09(1H, d), 7.91(1H, d), 7.77(1H, dd), 7.74(1H, d), 7.72(1H, d), 7.64(1H, dd), 7.59(1H, dd), |
| | 7.58(1H, s), 7.55 (2H, t), 7.5 (1H, t), 7.38 (1H, dd), 7.37 (2H, dd), 7.34 (1H, t), 7.29 |
| | (1H, d), 7.25 (1H, d), 7.24 (2H, d), |
| 1-111 | δ = 8.97(1H, s), 8.31(1H, d), 8.19(3H, d), 8.18(1H, d), 8.01(1H, d), 7.91(1H, d), 7.9(1H, d), |
| | 7.74(1H, d), 7.72(1H, d), 7.64(1H, dd), 7.62(2H, d), 7.58(3H, d), 7.5(4H, dd), 7.38(1H, dd), |
| | 7.34(1H, s), 7.29 (1H, t), 7.25 (1H, t), 7.24 (2H, dd), 7.2 (2H, dd), 7.08 (2H, t), 6.91 (1H, d), |
| | 6.48 (1H, d), |
| 1-120 | δ = 8.97(1H, s), 8.31(1H, d), 8.22(1H, d), 8.19(2H, d), 8.18(1H, d), 8.15(1H, d), 7.91(1H, d), |
| | 7.82(1H, d), 7.81(1H, d), 7.74(1H, dd), 7.72(1H, d), 7.69(1H, d), 7.64(1H, dd), 7.63(1H, dd), |
| | 7.59(1H, s), 7.58 (2H, t), 7.57 (1H, t), 7.55 (2H, dd), 7.5 (2H, dd), 7.38 (1H, t), 7.37 |
| | (2H, d), 7.29 (1H, d), 7.24 (2H, d), |
| 1-121 | δ = 8.97(1H, s), 8.29(1H, d), 8.19(2H, d), 8.18(1H, d), 8.06(1H, d), 7.72(1H, d), 7.64(1H, d), |
| | 7.59(1H, d), 7.58(1H, d), 7.5(1H, dd), 7.48(1H, d), 7.38(1H, d), 7.34(1H, dd), 7.29(1H, dd), |
| | 7.25(1H, s), 7.24 (4H, t), 7.2 (1H, t), 7.08 (4H, dd), 7 (2H, dd), 6.91 (1H, t), |
| 1-123 | δ = 8.97(1H, s), 8.29(1H, d), 8.19(2H, d), 8.18(1H, d), 8.06(1H, d), 7.75(2H, d), 7.72(1H, d), |
| | 7.64(1H, d), 7.58(1H, d), 7.55(2H, dd), 7.5(1H, d), 7.48(1H, d), 7.38(1H, dd), 7.37(2H, dd), |
| | 7.34(1H, s), 7.29 (1H, t), 7.25 (5H, t), 7.24 (2H, dd), 7.2 (1H, dd), 7.08 (2H, t), 6.91 (1H, d), |
| 1-132 | δ = 8.97(1H, s), 8.29(1H, d), 8.19(2H, d), 8.18(1H, d), 8.06(1H, d), 7.98(1H, d), 7.72(1H, d), |
| | 7.64(1H, d), 7.59(1H, d), 7.58(1H, dd), 7.54(1H, d), 7.5(1H, d), 7.48(1H, dd), 7.39(1H, dd), |
| | 7.38(1H, s), 7.34 (2H, t), 7.31 (1H, t), 7.29 (1H, dd), 7.25 (2H, dd), 7.24 (2H, t), 7.2 (1H, d), |
| | 7.08 (2H, d), 7 (1H, d), |
| 1-142 | δ = 8.97(1H, s), 8.29(1H, d), 8.19(2H, d), 8.18(1H, d), 8.06(1H, d), 7.82(1H, d), 7.75(2H, d), |
| | 7.72(1H, d), 7.69(1H, d), 7.64(1H, dd), 7.58(1H, d), 7.57(1H, d), 7.55(3H, dd), 7.5(1H, dd), |
| | 7.48(1H, s), 7.38 (1H, t), 7.37 (2H, t), 7.29 (1H, dd), 7.27 (1H, dd), 7.24 (2H, t), 7.2 (1H, d), |
| | 7.18 (1H, d), 7.17 (1H, d), |
| 1-145 | δ = 8.97(1H, s), 8.18(1H, d), 7.64(1H, d), 7.59(1H, d), 7.38(1H, d), 7.34(1H, d), 7.25(1H, d), |
| | 7.24(4H, d), 7.08(4H, d), 7(2H, dd), 6.91(1H, d), |
| 1-151 | δ = 7.38(1H, s) |
| 1-157 | δ = 8.31(1H, s), 8.19(2H, d), 8.09(1H, d), 7.91(1H, d), 7.74(1H, d), 7.72(1H, d), 7.58(1H, d), |
| | 7.55(2H, d), 7.5(1H, d), 7.37(2H, dd), 7.29(1H, d), 7.24(4H, d), 7.2(1H, dd), 7.08(4H, dd), |
| | 7(2H, s), |
| 1-160 | δ = 8.97(1H, s), 8.29(1H, d), 8.19(2H, d), 8.18(1H, d), 8.06(1H, d), 7.72(1H, d), 7.64(1H, d), |
| | 7.59(1H, d), 7.58(1H, d), 7.5(1H, dd), 7.48(1H, d), 7.38(1H, d), 7.34(1H, dd), 7.29(1H, dd), |
| | 7.25(1H, s), 7.2 (1H, t), 6.91 (1H, t), |
| 2-1 | δ = 8.19(2H, s), 7.88(1H, d), 7.83(1H, d), 7.79(1H, d), 7.65(2H, d), 7.58(2H, d), 7.5(2H, d), |
| | 7.34(1H, d), 7.25(1H, d), 7.24(4H, dd), 7.2(2H, d), 7.08(4H, d), 7(2H, dd), 6.91(1H, dd), |
| 2-2 | δ = 8.19(2H, s), 7.88(1H, d), 7.83(1H, d), 7.79(1H, d), 7.75(2H, d), 7.65(2H, d), 7.58(2H, d), |
| | 7.55(2H, d), 7.5(2H, d), 7.49(2H, dd), 7.41(1H, d), 7.37(2H, d), 7.34(1H, dd), 7.25(1H, dd), |
| | 7.24(2H, s), 7.2 (2H, t), 7.08 (2H, t), 7 (1H, dd), 6.91 (1H, dd), |
| 2-3 | δ = 8.19(2H, s), 7.88(1H, d), 7.83(1H, d), 7.79(1H, d), 7.75(2H, d), 7.65(2H, d), 7.58(2H, d), |
| | 7.55(2H, d), 7.5(2H, d), 7.49(2H, dd), 7.37(2H, d), 7.34(1H, d), 7.25(5H, dd), 7.24(2H, dd), |
| | 7.2(2H, s), 7.08 (2H, t), 6.91 (1H, t), |
| 2-4 | δ = 8.19(2H, s), 7.88(1H, d), 7.83(1H, d), 7.79(1H, d), 7.75(4H, d), 7.65(2H, d), 7.58(2H, d), |
| | 7.55(4H, d), 7.5(2H, d), 7.49(4H, dd), 7.37(4H, d), 7.34(1H, d), 7.25(1H, dd), 7.2(2H, dd), |
| | 6.91(1H, s), |
| 2-5 | δ = 8.19(2H, s), 7.88(1H, d), 7.83(1H, d), 7.79(1H, d), 7.75(2H, d), 7.65(2H, d), 7.58(2H, d), |
| | 7.55(1H, d), 7.5(2H, d), 7.49(2H, dd), 7.41(1H, d), 7.34(1H, d), 7.27(1H, dd), 7.25(1H, dd), |
| | 7.24(2H, s), 7.2 (2H, t), 7.18 (1H, t), 7.17 (1H, dd), 7.08 (2H, dd), 7 (1H, t), 6.91 (1H, d), |
| 2-6 | δ = 8.19(2H, s), 8.09(1H, d), 8.06(1H, d), 7.99(1H, d), 7.88(1H, d), 7.83(1H, d), 7.79(1H, d), |
| | 7.65(2H, d), 7.63(1H, d), 7.6(1H, dd), 7.58(2H, d), 7.55(3H, d), 7.5(2H, dd), 7.38(1H, dd), |
| | 7.37(2H, s), 7.34 (1H, t), 7.25 (1H, t), 7.24 (2H, dd), 7.2 (2H, dd), 7.08 (2H, t), 6.91 (1H, d), |
| 2-7 | δ = 8.19(2H, s), 7.88(1H, d), 7.83(1H, d), 7.79(1H, d), 7.75(4H, d), 7.65(2H, d), 7.58(2H, d), |
| | 7.55(3H, d), 7.5(2H, d), 7.49(4H, dd), 7.37(2H, d), 7.34(1H, d), 7.27(1H, dd), 7.25(1H, dd), |
| | 7.2(2H, s), 7.18 (1H, t), 7.17 (1H, t), 6.91 (1H, dd), |
| 2-9 | δ = 8.19(2H, s), 7.88(1H, d), 7.84(1H, d), 7.83(1H, d), 7.79(1H, d), 7.75(2H, d), 7.65(2H, d), |
| | 7.58(2H, d), 7.56(1H, d), 7.5(2H, dd), 7.49(2H, d), 7.4(1H, d), 7.34(1H, dd), 7.32(1H, dd), |
| | 7.25(1H, s), 7.24 (2H, t), 7.2 (2H, t), 7.17 (1H, dd), 7.11 (1H, dd), 7.08 (2H, t), 6.91 (1H, d), |
| 2-10 | δ = 8.95(1H, s), 8.5(1H, d), 8.2(1H, d), 8.19(2H, d), 8.09(1H, d), 7.88(1H, d), 7.83(1H, d), |
| | 7.79(1H, d), 7.77(1H, d), 7.65(2H, dd), 7.58(2H, d), 7.55(2H, d), 7.52(1H, dd), 7.5(2H, dd), |
| | 7.39(1H, s), 7.37 (2H, t), 7.34 (1H, t), 7.25 (1H, dd), 7.24 (2H, dd), 7.2 (2H, t), 7.08 (2H, d), |
| | 6.91 (1H, d), |
| 2-12 | δ = 8.19(2H, s), 7.88(1H, d), 7.83(1H, d), 7.79(1H, d), 7.78(1H, d), 7.75(2H, d), 7.71(1H, d), |
| | 7.65(2H, d), 7.58(2H, d), 7.55(2H, dd), 7.54(1H, d), 7.5(2H, d), 7.42(1H, dd), 7.37(2H, dd), |
| | 7.34(1H, s), 7.25 (5H, t), 7.2 (2H, t), 7.11 (1H, dd), 6.91 (1H, dd), |
| 2-13 | δ = 8.19(2H, s), 8.1(1H, d), 7.88(1H, d), 7.83(1H, d), 7.79(1H, d), 7.75(2H, d), 7.65(2H, d), |
| | 7.58(2H, d), 7.55(2H, d), 7.5(2H, dd), 7.37(3H, d), 7.34(1H, d), 7.25(5H, dd), 7.2(2H, dd), |
| | 7.08(2H, s), 6.91 (1H, t), |
| 2-14 | δ = 8.19(2H, s), 7.88(1H, d), 7.83(1H, d), 7.79(1H, d), 7.75(4H, d), 7.65(2H, d), 7.58(2H, d), |
| | 7.55(3H, d), 7.5(2H, d), 7.37(2H, dd), 7.34(1H, d), 7.27(1H, d), 7.25(5H, dd), 7.2(2H, dd), |
| | 7.18(1H, s), 7.17 (1H, t), 6.91 (1H, t), |
| 2-15 | δ = 8.19(2H, s), 7.98(1H, d), 7.88(1H, d), 7.83(1H, d), 7.79(1H, d), 7.65(2H, d), 7.58(2H, d), |
| | 7.54(1H, d), 7.5(2H, d), 7.39(1H, dd), 7.34(2H, d), 7.31(1H, d), 7.25(2H, dd), 7.24(2H, dd), |
| | 7.2(2H, s), 7.08 (2H, t), 7 (1H, t), 6.91 (2H, dd), |
| 2-16 | δ = 8.22(1H, s), 8.19(2H, d), 7.98(1H, d), 7.88(1H, d), 7.83(1H, d), 7.79(1H, d), 7.65(2H, d), |
| | 7.58(2H, d), 7.56(1H, d), 7.54(1H, dd), 7.5(2H, d), 7.39(1H, d), 7.34(1H, dd), 7.31(1H, dd), |
| | 7.25(1H, s), 7.24 (2H, t), 7.2 (2H, t), 7.08 (2H, dd), 7 (1H, dd), 6.97 (1H, t), 6.91 (1H, d), |
| 2-17 | δ = 8.19(2H, s), 8.03(1H, d), 7.98(1H, d), 7.88(1H, d), 7.83(1H, d), 7.8(1H, d), 7.79(1H, d), |
| | 7.65(2H, d), 7.58(2H, d), 7.54(1H, dd), 7.5(2H, d), 7.39(1H, d), 7.34(1H, dd), 7.31(1H, dd), |
| | 7.25(1H, s), 7.24 (2H, t), 7.2 (2H, t), 7.08 (2H, dd), 7 (1H, dd), 6.91 (2H, t), |
| 2-19 | δ = 8.55(1H, s), 8.24(1H, d), 8.19(2H, d), 7.94(1H, d), 7.88(1H, d), 7.83(1H, d), 7.79(1H, d), |
| | 7.65(2H, d), 7.62(2H, d), 7.58(3H, dd), 7.5(4H, d), 7.35(1H, d), 7.34(1H, dd), 7.27(1H, dd), |
| | 7.25(2H, s), 7.24 (2H, t), 7.2 (2H, t), 7.16 (1H, dd), 7.08 (2H, dd), 6.91 (1H, t), |
| 2-20 | δ = 8.45(1H, s), 8.19(2H, d), 7.95(1H, d), 7.93(1H, d), 7.88(1H, d), 7.85(1H, d), 7.83(1H, d), |
| | 7.79(1H, d), 7.65(2H, d), 7.58(2H, dd), 7.56(1H, d), 7.5(2H, d), 7.49(1H, dd), 7.41(1H, dd), |
| | 7.34(1H, s), 7.25 (1H, t), 7.24 (2H, t), 7.2 (2H, dd), 7.08 (2H, dd), 7 (1H, t), 6.91 (1H, d), |
| 2-22 | δ = 8.19(2H, s), 7.88(1H, d), 7.83(1H, d), 7.82(1H, d), 7.79(1H, d), 7.69(1H, d), 7.65(2H, d), |
| | 7.58(2H, d), 7.57(1H, d), 7.55(2H, dd), 7.5(2H, d), 7.37(2H, d), 7.24(4H, dd), 7.2(2H, dd), |
| | 7.08(4H, s), 7 (2H, t), |
| 2-23 | δ = 8.19(2H, s), 7.88(1H, d), 7.84(1H, d), 7.83(1H, d), 7.82(1H, d), 7.79(1H, d), 7.69(1H, d), |
| | 7.65(2H, d), 7.58(2H, d), 7.57(1H, dd), 7.56(1H, d), 7.5(2H, d), 7.4(1H, dd), 7.32(1H, dd), |
| | 7.24(4H, s), 7.2 (2H, t), 7.17 (1H, t), 7.11 (1H, dd), 7.08 (4H, dd), |
| 2-25 | δ = 8.19(2H, s), 7.88(1H, d), 7.83(1H, d), 7.82(1H, d), 7.79(1H, d), 7.75(2H, d), 7.69(1H, d), |
| | 7.65(2H, d), 7.58(2H, d), 7.57(1H, dd), 7.55(4H, d), 7.5(2H, d), 7.49(2H, dd), 7.37(4H, dd), |
| | 7.24(2H, s), 7.2 (2H, t), 7.08 (2H, t), |
| 2-28 | δ = 8.19(2H, s), 7.88(1H, d), 7.84(1H, d), 7.83(1H, d), 7.82(1H, d), 7.79(1H, d), 7.78(1H, d), |
| | 7.71(1H, d), 7.69(1H, d), 7.65(2H, dd), 7.58(2H, d), 7.57(1H, d), 7.56(1H, dd), 7.54(1H, dd), |
| | 7.5(2H, s), 7.42 (1H, t), 7.4 (2H, t), 7.32 (1H, dd), 7.24 (2H, dd), 7.2 (2H, t), 7.17 |
| | (1H, d), 7.11 (2H, d), 7.08 (2H, d), |
| 2-29 | δ = 8.19(2H, s), 7.91(1H, d), 7.88(1H, d), 7.84(1H, d), 7.83(1H, d), 7.79(1H, d), 7.58(2H, d), |
| | 7.5(2H, d), 7.34(1H, d), 7.25(1H, dd), 7.24(4H, d), 7.2(2H, d), 7.08(4H, dd), 7(2H, dd), |
| | 6.91(1H, s), |
| 2-31 | δ = 8.19(2H, s), 7.91(1H, d), 7.88(1H, d), 7.84(1H, d), 7.83(1H, d), 7.79(1H, d), 7.75(2H, d), |
| | 7.58(2H, d), 7.55(2H, d), 7.5(2H, dd), 7.49(2H, d), 7.37(2H, d), 7.34(1H, dd), 7.25(5H, dd), |
| | 7.24(2H, s), 7.2 (2H, t), 7.08 (2H, t), 6.91 (1H, dd), |
| 2-38 | δ = 8.95(1H, s), 8.5(1H, d), 8.2(1H, d), 8.19(2H, d), 8.09(1H, d), 7.91(1H, d), 7.88(1H, d), |
| | 7.84(1H, d), 7.83(1H, d), 7.79(1H, dd), 7.77(1H, d), 7.58(2H, d), 7.55(2H, dd), 7.52(1H, dd), |
| | 7.5(2H, s), 7.39 (1H, t), 7.37 (2H, t), 7.34 (1H, dd), 7.25 (1H, dd), 7.24 (2H, t), 7.2 |
| | (2H, d), 7.08 (2H, d), 6.91 (1H, d), |
| 2-46 | δ = 8.19(3H, s), 8.01(1H, d), 7.91(1H, d), 7.9(1H, d), 7.88(1H, d), 7.84(1H, d), 7.83(1H, d), |
| | 7.79(1H, d), 7.62(2H, d), 7.58(4H, dd), 7.5(5H, d), 7.34(1H, d), 7.25(1H, dd), 7.24(2H, dd), |
| | 7.2(3H, s), 7.08 (2H, t), 6.91 (1H, t), 6.48 (1H, dd), |
| 2-52 | δ = 8.19(2H, s), 7.91(1H, d), 7.88(1H, d), 7.84(2H, d), 7.83(1H, d), 7.82(1H, d), 7.79(1H, d), |
| | 7.75(2H, d), 7.69(1H, d), 7.58(2H, dd), 7.57(1H, d), 7.56(1H, d), 7.55(2H, dd), 7.5(2H, dd), |
| | 7.4(1H, s), 7.37 (2H, t), 7.32 (1H, t), 7.2 (2H, dd), 7.17 (1H, dd), 7.11 (1H, t), 7.08 (2H, d), |
| 2-57 | δ = 8.19(2H, s), 7.91(1H, d), 7.88(1H, d), 7.83(1H, d), 7.79(1H, d), 7.72(1H, d), 7.68(1H, d), |
| | 7.58(1H, d), 7.56(1H, d), 7.5(1H, dd), 7.34(1H, d), 7.29(1H, d), 7.25(1H, dd), 7.24(4H, dd), |
| | 7.2(1H, s), 7.08 (4H, t), 7 (2H, t), 6.91 (1H, dd), |
| 2-60 | δ = 8.19(2H, s), 7.91(1H, d), 7.88(1H, d), 7.83(1H, d), 7.79(1H, d), 7.75(4H, d), 7.72(1H, d), |
| | 7.68(1H, d), 7.58(1H, d), 7.56(1H, dd), 7.55(4H, d), 7.5(1H, d), 7.49(4H, dd), 7.37(4H, dd), |
| | 7.34(1H, s), 7.29 (1H, t), 7.25 (1H, t), 7.2 (1H, dd), 6.91 (1H, dd), |
| 2-68 | δ = 8.19(2H, s), 7.91(1H, d), 7.88(1H, d), 7.83(1H, d), 7.79(1H, d), 7.78(1H, d), 7.75(2H, d), |
| | 7.72(1H, d), 7.71(1H, d), 7.68(1H, dd), 7.58(1H, d), 7.56(1H, d), 7.55(2H, dd), 7.54(1H, dd), |
| | 7.5(1H, s), 7.42 (1H, t), 7.37 (2H, t), 7.34 (1H, dd), 7.29 (1H, dd), 7.25 (5H, t), 7.2 |
| | (1H, d), 7.11 (1H, d), 6.91 (1H, d), |
| 2-70 | δ = 8.19(2H, s), 7.91(1H, d), 7.88(1H, d), 7.83(1H, d), 7.79(1H, d), 7.75(4H, d), 7.72(1H, d), |
| | 7.68(1H, d), 7.58(1H, d), 7.56(1H, dd), 7.55(3H, d), 7.5(1H, d), 7.37(2H, dd), 7.34(1H, dd), |
| | 7.29(1H, s), 7.27 (1H, t), 7.25 (5H, t), 7.2 (1H, dd), 7.18 (1H, dd), 7.17 (1H, t), 6.91 (1H, d), |
| 2-74 | δ = 8.19(3H, s), 8.01(1H, d), 7.91(1H, d), 7.9(1H, d), 7.88(1H, d), 7.83(1H, d), 7.79(1H, d), |
| | 7.72(1H, d), 7.68(1H, d), 7.62(2H, dd), 7.58(3H, d), 7.56(1H, d), 7.5(4H, dd), 7.34(1H, dd), |
| | 7.29(1H, s), 7.25 (1H, t), 7.24 (2H, t), 7.2 (2H, dd), 7.08 (2H, dd), 6.91 (1H, t), 6.48 (1H, d), |
| 2-78 | δ = 8.19(2H, s), 7.91(1H, d), 7.88(1H, d), 7.83(1H, d), 7.82(1H, d), 7.79(1H, d), 7.72(1H, d), |
| | 7.69(1H, d), 7.68(1H, d), 7.58(1H, dd), 7.57(1H, d), 7.56(1H, d), 7.55(2H, dd), 7.5(1H, dd), |
| | 7.37(2H, s), 7.29 (1H, t), 7.24 (4H, t), 7.2 (1H, dd), 7.08 (4H, dd), 7 (2H, t), |
| 2-85 | δ = 8.19(2H, s), 7.99(1H, d), 7.89(1H, d), 7.88(1H, d), 7.83(1H, d), 7.79(1H, d), 7.77(1H, d), |
| | 7.72(1H, d), 7.58(1H, d), 7.5(1H, dd), 7.34(1H, d), 7.29(1H, d), 7.25(1H, dd), 7.24(4H, dd), |
| | 7.2(1H, s), 7.08 (4H, t), 7 (2H, t), 6.91 (1H, dd), |
| 2-91 | δ = 8.19(2H, s), 7.99(1H, d), 7.89(1H, d), 7.88(1H, d), 7.83(1H, d), 7.79(1H, d), 7.77(1H, d), |
| | 7.75(4H, d), 7.72(1H, d), 7.58(1H, dd), 7.55(3H, d), 7.5(1H, d), 7.49(4H, dd), 7.37(2H, dd), |
| | 7.34(1H, s), 7.29 (1H, t), 7.27 (1H, t), 7.25 (1H, dd), 7.2 (1H, dd), 7.18 (1H, t), 7.17 (1H, d), |
| | 6.91 (1H, d), |
| 2-100 | δ = 8.22(1H, s), 8.19(2H, d), 7.99(1H, d), 7.98(1H, d), 7.89(1H, d), 7.88(1H, d), 7.83(1H, d), |
| | 7.79(1H, d), 7.77(1H, d), 7.72(1H, dd), 7.58(1H, d), 7.56(1H, d), 7.54(1H, dd), 7.5(1H, dd), |
| | 7.39(1H, s), 7.34 (1H, t), 7.31 (1H, t), 7.29 (1H, dd), 7.25 (1H, dd), 7.24 (2H, t), 7.2 (1H, d), |
| | 7.08 (2H, d), 7 (1H, d), |
| 2-105 | δ = 8.45(1H, s), 8.19(2H, d), 8.01(1H, d), 7.99(1H, d), 7.93(1H, d), 7.89(1H, d), 7.88(1H, d), |
| | 7.83(1H, d), 7.79(1H, d), 7.77(1H, dd), 7.72(1H, d), 7.64(1H, d), 7.58(1H, dd), 7.56(1H, dd), |
| | 7.5(1H, s), 7.49 (1H, t), 7.43 (1H, t), 7.34 (1H, dd), 7.29 (1H, dd), 7.25 (1H, t), 7.24 |
| | (2H, d), 7.2 (1H, d), 7.08 (2H, d), |
| 2-108 | δ = 8.19(2H, s), 7.99(1H, d), 7.89(1H, d), 7.88(1H, d), 7.84(1H, d), 7.83(1H, d), 7.82(1H, d), |
| | 7.79(1H, d), 7.77(1H, d), 7.75(2H, dd), 7.72(1H, d), 7.69(1H, d), 7.58(1H, dd), 7.57(1H, dd), |
| | 7.56(1H, s), 7.55 (2H, t), 7.5 (1H, t), 7.4 (1H, dd), 7.37 (2H, dd), 7.32 (1H, t), 7.29 |
| | (1H, d), 7.2 (1H, d), 7.17 (1H, d), |
| 2-109 | δ = 8.19(2H, s), 7.99(1H, d), 7.89(1H, d), 7.88(1H, d), 7.83(1H, d), 7.82(1H, d), 7.79(1H, d), |
| | 7.77(1H, d), 7.75(2H, d), 7.72(1H, dd), 7.69(1H, d), 7.58(1H, d), 7.57(1H, dd), 7.55(4H, dd), |
| | 7.5(1H, s), 7.49 (2H, t), 7.37 (4H, t), 7.29 (1H, dd), 7.24 (2H, dd), 7.2 (1H, t), 7.08 |
| | (2H, d), |
| 2-113 | δ = 8.31(1H, s), 8.19(2H, d), 7.91(1H, d), 7.88(1H, d), 7.83(1H, d), 7.79(1H, d), 7.74(1H, d), |
| | 7.72(1H, d), 7.58(1H, d), 7.5(1H, dd), 7.34(1H, d), 7.29(1H, d), 7.25(1H, dd), 7.24(4H, dd), |
| | 7.2(1H, s), 7.08 (4H, t), 7 (2H, t), 6.91 (1H, dd), |
| 2-120 | δ = 8.31(1H, s), 8.19(2H, d), 8.1(1H, d), 7.91(1H, d), 7.88(1H, d), 7.83(1H, d), 7.79(1H, d), |
| | 7.75(2H, d), 7.74(1H, d), 7.72(1H, dd), 7.58(1H, d), 7.55(2H, d), 7.5(1H, dd), 7.49(2H, dd), |
| | 7.43(2H, s), 7.39 (1H, t), 7.37 (3H, t), 7.34 (1H, dd), 7.29 (1H, dd), 7.25 (1H, t), 7.2 (1H, d), |
| | 7.14 (1H, d), 7.08 (2H, d), |
| 2-126 | δ = 8.31(1H, s), 8.19(2H, d), 7.91(1H, d), 7.88(1H, d), 7.83(1H, d), 7.79(1H, d), 7.75(4H, d), |
| | 7.74(1H, d), 7.72(1H, d), 7.58(1H, dd), 7.55(3H, d), 7.5(1H, d), 7.37(2H, dd), 7.34(1H, dd), |
| | 7.29(1H, s), 7.27 (1H, t), 7.25 (5H, t), 7.2 (1H, dd), 7.18 (1H, dd), 7.17 (1H, t), 6.91 (1H, d), |
| 2-131 | δ = 8.55(1H, s), 8.31(1H, d), 8.24(1H, d), 8.19(2H, d), 7.94(1H, d), 7.91(1H, d), 7.88(1H, d), |
| | 7.83(1H, d), 7.79(1H, d), 7.74(1H, dd), 7.72(1H, d), 7.62(2H, d), 7.58(2H, dd), 7.5(3H, dd), |
| | 7.35(1H, s), 7.34 (1H, t), 7.29 (1H, t), 7.27 (1H, dd), 7.25 (2H, dd), 7.24 (2H, t), 7.2 (1H, d), |
| | 7.16 (1H, d), 7.08 (2H, d), |
| 2-134 | δ = 8.31(1H, s), 8.19(2H, d), 7.91(1H, d), 7.88(1H, d), 7.83(1H, d), 7.82(1H, d), 7.79(1H, d), |
| | 7.74(1H, d), 7.72(1H, d), 7.69(1H, dd), 7.58(1H, d), 7.57(1H, d), 7.55(2H, dd), 7.5(1H, dd), |
| | 7.37(2H, s), 7.29 (1H, t), 7.24 (4H, t), 7.2 (1H, dd), 7.08 (4H, dd), 7 (2H, t), |
| 2-139 | δ = 8.31(1H, s), 8.19(2H, d), 7.91(1H, d), 7.88(1H, d), 7.83(1H, d), 7.82(1H, d), 7.79(1H, d), |
| | 7.78(1H, d), 7.74(1H, d), 7.72(1H, dd), 7.71(1H, d), 7.69(1H, d), 7.58(1H, dd), 7.57(1H, dd), |
| | 7.55(2H, s), 7.54 (1H, t), 7.5 (1H, t), 7.45 (1H, dd), 7.42 (1H, dd), 7.4 (1H, t), 7.37 |
| | (2H, d), 7.29 (1H, d), 7.24 (2H, d), |
| 2-141 | δ = 8.29(1H, s), 8.19(2H, d), 8.06(1H, d), 7.88(1H, d), 7.83(1H, d), 7.79(1H, d), 7.72(1H, d), |
| | 7.58(1H, d), 7.5(1H, d), 7.48(1H, dd), 7.34(1H, d), 7.29(1H, d), 7.25(1H, dd), 7.24(4H, dd), |
| | 7.2(1H, s), 7.08 (4H, t), 7 (2H, t), 6.91 (1H, dd), |
| 2-147 | δ = 8.29(1H, s), 8.19(2H, d), 8.06(1H, d), 7.88(1H, d), 7.83(1H, d), 7.79(1H, d), 7.75(4H, d), |
| | 7.72(1H, d), 7.58(1H, d), 7.55(3H, dd), 7.5(1H, d), 7.49(4H, d), 7.48(1H, dd), 7.37(2H, dd), |
| | 7.34(1H, s), 7.29 (1H, t), 7.27 (1H, t), 7.25 (1H, dd), 7.2 (1H, dd), 7.18 (1H, t), 7.17 (1H, d), |
| | 6.91 (1H, d), |
| 2-160 | δ = 8.45(1H, s), 8.29(1H, d), 8.19(2H, d), 8.06(1H, d), 7.95(1H, d), 7.93(1H, d), 7.88(1H, d), |
| | 7.85(1H, d), 7.83(1H, d), 7.79(1H, dd), 7.72(1H, d), 7.58(1H, d), 7.56(1H, dd), 7.5(1H, dd), |
| | 7.49(1H, s), 7.48 (1H, t), 7.41 (1H, t), 7.34 (1H, dd), 7.29 (1H, dd), 7.25 (1H, t), 7.24 |
| | (2H, d), 7.2 (1H, d), 7.08 (2H, d), |
| 2-161 | δ = 8.29(1H, s), 8.19(2H, d), 8.06(1H, d), 7.88(1H, d), 7.83(1H, d), 7.79(1H, d), 7.72(1H, d), |
| | 7.58(1H, d), 7.5(1H, d), 7.48(1H, dd), 7.34(1H, d), 7.29(1H, d), 7.25(1H, dd), 7.2(1H, dd), |
| | 7.11(1H, s), 6.91 (1H, t), |
| 2-178 | δ = 8.19(2H, s), 7.99(1H, d), 7.89(1H, d), 7.88(1H, d), 7.83(1H, d), 7.82(1H, d), 7.79(1H, d), |
| | 7.77(1H, d), 7.72(1H, d), 7.69(1H, dd), 7.58(1H, d), 7.57(1H, d), 7.5(1H, dd), 7.29(1H, dd), |
| | 7.2(1H, s), 2.25 (1H, t), |

TABLE 8

| Compound | FD-Mass | Compound | FD-Mass |
|---|---|---|---|
| 1-1 | m/z = 624.22 (C₄₆H₂₈N₂O, 624.74) | 1-2 | m/z = 700.25 (C₅₂H₃₂N₂O, 700.84) |
| 1-3 | m/z = 776.28 (C₅₈H₃₆N₂O, 776.94) | 1-4 | m/z = 776.28 (C₅₈H₃₆N₂O, 776.94) |
| 1-5 | m/z = 700.25 (C₅₂H₃₂N₂O, 700.84) | 1-6 | m/z = 750.27 (C₅₆H₃₄N₂O, 750.90) |
| 1-7 | m/z = 776.28 (C₅₈H₃₆N₂O, 776.94) | 1-8 | m/z = 776.28 (C₅₈H₃₆N₂O, 776.94) |
| 1-9 | m/z = 750.27 (C₅₆H₃₄N₂O, 750.90) | 1-10 | m/z = 750.27 (C₅₆H₃₄N₂O, 750.90) |
| 1-11 | m/z = 776.28 (C₅₈H₃₆N₂O, 776.94) | 1-12 | m/z = 714.23 (C₅₂H₃₀N₂O₂, 714.82) |
| 1-13 | m/z = 714.23 (C₅₂H₃₀N₂O₂, 714.82) | 1-14 | m/z = 714.23 (C₅₂H₃₀N₂O₂, 714.82) |
| 1-15 | m/z = 789.28 (C₅₈H₃₅N₃O, 789.94) | 1-16 | m/z = 789.28 (C₅₈H₃₅N₃O, 789.94) |
| 1-17 | m/z = 730.21 (C₅₂H₃₀N₂OS, 730.88) | 1-18 | m/z = 730.21 (C₅₂H₃₀N₂OS, 730.88) |
| 1-19 | m/z = 700.25 (C₅₂H₃₂N₂O, 700.84) | 1-20 | m/z = 750.27 (C₅₆H₃₄N₂O, 750.90) |
| 1-21 | m/z = 776.28 (C₅₈H₃₆N₂O, 776.94) | 1-22 | m/z = 776.28 (C₅₈H₃₆N₂O, 776.94) |
| 1-23 | m/z = 750.27 (C₅₆H₃₄N₂O, 750.90) | 1-25 | m/z = 624.22 (C₄₆H₂₈N₂O, 624.74) |
| 1-28 | m/z = 776.28 (C₅₈H₃₆N₂O, 776.94) | 1-30 | m/z = 750.27 (C₅₆H₃₄N₂O, 750.90) |
| 1-37 | m/z = 714.23 (C₅₂H₃₀N₂O₂, 714.82) | 1-40 | m/z = 789.28 (C₅₈H₃₅N₃O, 789.94) |
| 1-44 | m/z = 750.27 (C₅₆H₃₄N₂O, 750.90) | 1-45 | m/z = 776.28 (C₅₈H₃₆N₂O, 776.94) |
| 1-49 | m/z = 624.22 (C₄₆H₂₈N₂O, 624.74) | 1-51 | m/z = 776.28 (C₅₈H₃₆N₂O, 776.94) |
| 1-55 | m/z = 776.28 (C₅₈H₃₆N₂O, 776.94) | 1-59 | m/z = 776.28 (C₅₈H₃₆N₂O, 776.94) |
| 1-65 | m/z = 730.21 (C₅₂H₃₀N₂OS, 730.88) | 1-68 | m/z = 750.27 (C₅₆H₃₄N₂O, 750.90) |
| 1-73 | m/z = 750.27 (C₅₆H₃₄N₂O, 750.90) | 1-78 | m/z = 750.27 (C₅₆H₃₄N₂O, 750.90) |
| 1-88 | m/z = 789.28 (C₅₈H₃₅N₃O, 789.94) | 1-92 | m/z = 750.27 (C₅₆H₃₄N₂O, 750.90) |
| 1-94 | m/z = 776.28 (C₅₈H₃₆N₂O, 776.94) | 1-97 | m/z = 624.22 (C₄₆H₂₈N₂O, 624.74) |
| 1-100 | m/z = 776.28 (C₅₈H₃₆N₂O, 776.94) | 1-106 | m/z = 750.27 (C₅₆H₃₄N₂O, 750.90) |
| 1-111 | m/z = 789.28 (C₅₈H₃₅N₃O, 789.94) | 1-120 | m/z = 750.27 (C₅₆H₃₄N₂O, 750.90) |
| 1-121 | m/z = 624.22 (C₄₆H₂₈N₂O, 624.74) | 1-123 | m/z = 776.28 (C₅₈H₃₆N₂O, 776.94) |
| 1-132 | m/z = 714.23 (C₅₂H₃₀N₂O₂, 714.82) | 1-142 | m/z = 776.28 (C₅₈H₃₆N₂O, 776.94) |
| 1-145 | m/z = 634.28 (C₄₆H₁₈D₁₀N₂O, 634.80) | 1-151 | m/z = 783.47 (C₅₆HD₃₃N₂O, 784.10) |
| 1-157 | m/z = 707.88 (C₅₂H₂₅D₇N₂O, 707.30) | 1-160 | m/z = 794.40 (C₅₈H₁₈D₁₈N₂O, 795.05) |
| 2-1 | m/z = 574.20 (C₄₂H₂₆N₂O, 574.68) | 2-2 | m/z = 650.24 (C₄₈H₃₀N₂O, 650.78) |
| 2-3 | m/z = 726.27 (C₅₄H₃₄N₂O, 726.88) | 2-4 | m/z = 726.27 (C₅₄H₃₄N₂O, 726.88) |
| 2-5 | m/z = 650.24 (C₄₈H₃₀N₂O, 650.78) | 2-6 | m/z = 700.25 (C₅₂H₃₂N₂O, 700.84) |
| 2-7 | m/z = 726.27 (C₅₄H₃₄N₂O, 726.88) | 2-9 | m/z = 700.25 (C₅₂H₃₂N₂O, 700.84) |
| 2-10 | m/z = 700.25 (C₅₂H₃₂N₂O, 700.84) | 2-12 | m/z = 776.28 (C₅₈H₃₆N₂O, 776.94) |
| 2-13 | m/z = 802.30 (C₆₀H₃₈N₂O, 802.98) | 2-14 | m/z = 802.30 (C₆₀H₃₈N₂O, 802.98) |
| 2-15 | m/z = 664.22 (C₄₈H₂₈N₂O₂, 664.76) | 2-16 | m/z = 664.22 (C₄₈H₂₈N₂O₂, 664.76) |
| 2-17 | m/z = 664.22 (C₄₈H₂₈N₂O₂, 664.76) | 2-19 | m/z = 739.26 (C₅₄H₃₃N₃O, 739.88) |
| 2-20 | m/z = 680.19 (C₄₈H₂₈N₂OS, 680.83) | 2-22 | m/z = 650.24 (C₄₈H₃₀N₂O, 650.78) |
| 2-23 | m/z = 700.25 (C₅₂H₃₂N₂O, 700.84) | 2-25 | m/z = 726.27 (C₅₄H₃₄N₂O, 726.88) |
| 2-28 | m/z = 750.27 (C₅₆H₃₄N₂O, 750.90) | 2-29 | m/z = 574.20 (C₄₂H₂₆N₂O, 574.68) |
| 2-31 | m/z = 726.27 (C₅₄H₃₄N₂O, 726.88) | 2-38 | m/z = 700.25 (C₅₂H₃₂N₂O, 700.84) |
| 2-46 | m/z = 739.26 (C₅₄H₃₃N₃O, 739.88) | 2-52 | m/z = 776.28 (C₅₈H₃₆N₂O, 776.94) |
| 2-57 | m/z = 574.20 (C₄₂H₂₆N₂O, 574.68) | 2-60 | m/z = 726.27 (C₅₄H₃₄N₂O, 726.88) |
| 2-68 | m/z = 776.28 (C₅₈H₃₆N₂O, 776.94) | 2-70 | m/z = 802.30 (C₆₀H₃₈N₂O, 802.98) |
| 2-74 | m/z = 739.26 (C₅₄H₃₃N₃O, 739.88) | 2-78 | m/z = 650.24 (C₄₈H₃₀N₂O, 650.78) |
| 2-85 | m/z = 574.20 (C₄₂H₂₆N₂O, 574.68) | 2-91 | m/z = 726.27 (C₅₄H₃₄N₂O, 726.88) |
| 2-100 | m/z = 664.22 (C₄₈H₂₈N₂O₂, 664.76) | 2-105 | m/z = 680.19 (C₄₈H₂₈N₂OS, 680.83) |
| 2-108 | m/z = 776.28 (C₅₈H₃₆N₂O, 776.94) | 2-109 | m/z = 726.27 (C₅₄H₃₄N₂O, 726.88) |
| 2-113 | m/z = 574.20 (C₄₂H₂₆N₂O, 574.68) | 2-120 | m/z = 726.27 (C₅₄H₃₄N₂O, 726.88) |
| 2-126 | m/z = 802.30 (C₆₀H₃₈N₂O, 802.98) | 2-131 | m/z = 739.26 (C₅₄H₃₃N₃O, 739.88) |
| 2-134 | m/z = 650.24 (C₄₈H₃₀N₂O, 650.78) | 2-139 | m/z = 700.25 (C₅₂H₃₂N₂O, 700.84) |
| 2-141 | m/z = 574.20 (C₄₂H₂₆N₂O, 574.68) | 2-147 | m/z = 726.27 (C₅₄H₃₄N₂O, 726.88) |
| 2-160 | m/z = 680.19 (C₄₈H₂₈N₂OS, 680.83) | 2-161 | m/z = 691.26 (C₄₈H₁₇D₁₁N₂OS, 691.89) |
| 2-178 | m/z = 677.33 (C₄₉H₁₉D₁₃N₂O, 677.89) | 2-29 | m/z = 574.20 (C₄₂H₂₆N₂O, 574.68) |

### Preparation Example 3. Preparation of Compound 3-59

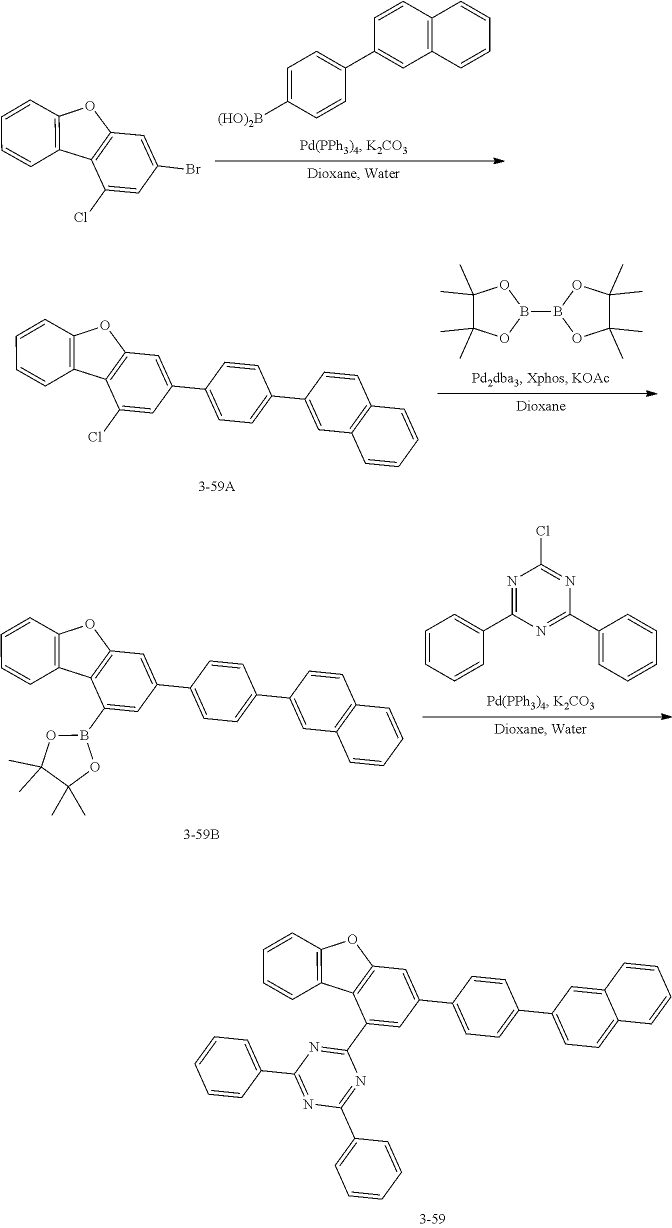

### Preparation Example 3-1. Preparation of Compound 3-59A

3-bromo-1-chlorodibenzo[b,d]furan (10.0 g, 35.7 mmol), (4-(naphthalen-2-yl)phenyl)boronic acid (13.3 g, 53.6 mmol), Pd(PPh₃)₄ (2.1 g, 1.5 mmol) and K₂CO₃ (9.9 g, 71.4 mmol) were dissolved in 1,4-dioxane (100 mL) and distilled water (20 mL), and then the mixture was refluxed for 3 hours.

After the reaction was completed, the reaction material was filtered under reduced pressure at room temperature, and then the solvent of the filtrate was removed using a rotary evaporator. After that, the result was dissolved in dichloromethane (DCM) and purified by silica, and then recrystallized with dichloromethane and methanol to obtain Compound 3-59A (11.8 g, yield 82%).

### Preparation Example 3-2. Preparation of Compound 3-59B

Compound 3-59A (11.8 g, 39.3 mmol), bis(pinacolato)diboron (20.0 g, 78.6 mmol), Pd(PPh₃)₄ (2.3 g, 2.0 mmol) and KOAc (7.7 g, 78.6 mmol) were dissolved in 1,4-dioxane (120 mL), and then the mixture was refluxed for 3 hours.

After the reaction was completed, the reaction material was filtered under reduced pressure at room temperature, and then the solvent of the filtrate was removed using a rotary evaporator. After that, the result was dissolved in dichloromethane (DCM) and purified by purified by silica, and then recrystallized with dichloromethane and methanol to obtain Compound 3-59B (17.5 g, yield 90%).

### Preparation Example 3-3. Preparation of Compound 3-59

Compound 3-59B (17.5 g, 35.4 mmol), 2-chloro-4,6-diphenyl-1,3,5-triazine (9.5 g, 35.4 mmol), Pd₂(dba)₃ (1.6 g, 1.8 mmol), Xphos (1.7 g, 3.6 mmol) and K₂CO₃ (9.8 g, 70.8 mmol) were dissolved in 1,4-dioxane (180 mL) and distilled water (36 mL), and then the mixture was refluxed for 3 hours.

After the reaction was completed, the reaction material was filtered under reduced pressure at room temperature. After that, the result was dissolved in 1,2-dichlorobenzene (DCB) and purified by silica, and then recrystallized with dichloromethane and methanol to obtain target Compound 3-59 (17.7 g, yield 83%).

Target compounds were prepared as in the following Table 9 in the same manner as in Preparation Example 3, except that Compound a of the following Table 9 was used instead of 3-bromo-1-chlorodibenzo[b,d]furan, Compound b of the following Table 9 was used instead of (4-(naphthalen-2-yl)phenyl)boronic acid, and Compound c of the following Table 9 was used instead of 2-chloro-4,6-diphenyl-1,3,5-triazine.

TABLE 9

| Com- | | | | | |
|---|---|---|---|---|---|
| pound | | | | | |
| No. | Compound a | Compound b | Compound c | Target Compound | Yield |
| 3-59 | 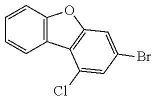 [SMILES: Clc1cc(Br)cc2oc3ccccc3c12] | 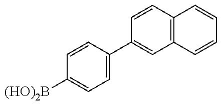 [SMILES: OB(O)c1ccc(-c2ccc3ccccc3c2)cc1] | 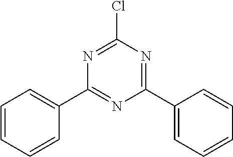 [SMILES: Clc1nc(-c2ccccc2)nc(-c2ccccc2)n1] | 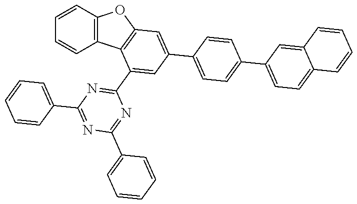 [SMILES: c1ccc(-c2nc(-c3ccccc3)nc(-c3cc(-c4ccc(-c5ccc6ccccc6c5)cc4)cc4oc5ccccc5c34)n2)cc1] | 83% |
| 3-62 | 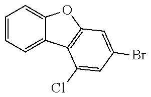 [SMILES: Clc1cc(Br)cc2oc3ccccc3c12] | 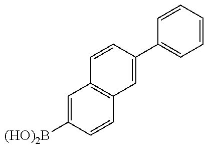 [SMILES: OB(O)c1ccc2cc(-c3ccccc3)ccc2c1] | 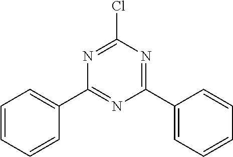 [SMILES: Clc1nc(-c2ccccc2)nc(-c2ccccc2)n1] | 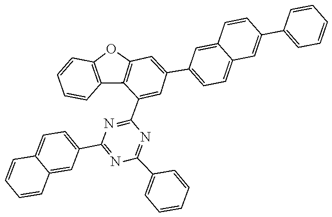 [SMILES: c1ccc(-c2ccc3cc(-c4cc(-c5nc(-c6ccccc6)nc(-c6ccc7ccccc7c6)n5)c5c(c4)oc4ccccc45)ccc3c2)cc1] | 75% |
| 3-97 | 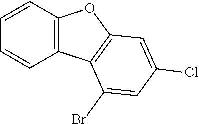 [SMILES: Clc1cc(Br)c2c(c1)oc1ccccc12] | 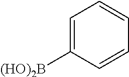 | 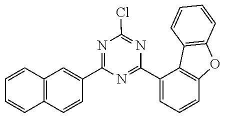 [SMILES: Clc1nc(-c2ccc3ccccc3c2)nc(-c2cccc3oc4ccccc4c23)n1] | 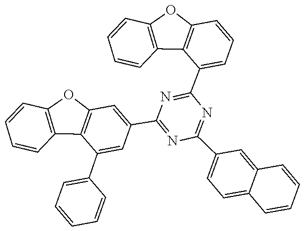 [SMILES: c1ccc(-c2cc(-c3nc(-c4ccc5ccccc5c4)nc(-c4cccc5oc6ccccc6c45)n3)cc3oc4ccccc4c23)cc1] | 72% |
| 3-132 | 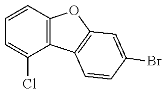 [SMILES: Clc1cccc2oc3cc(Br)ccc3c12] | 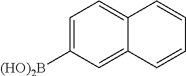 [SMILES: OB(O)c1ccc2ccccc2c1] | 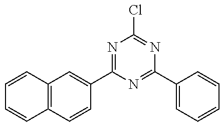 [SMILES: Clc1nc(-c2ccccc2)nc(-c2ccc3ccccc3c2)n1] | 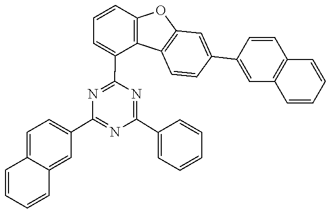 [SMILES: c1ccc(-c2nc(-c3ccc4ccccc4c3)nc(-c3cccc4oc5cc(-c6ccc7ccccc7c6)ccc5c34)n2)cc1] | 78% |

### Preparation Example 4. Preparation of Compound 3-216

### Preparation Example 4-1. Preparation of Compound 3-216A

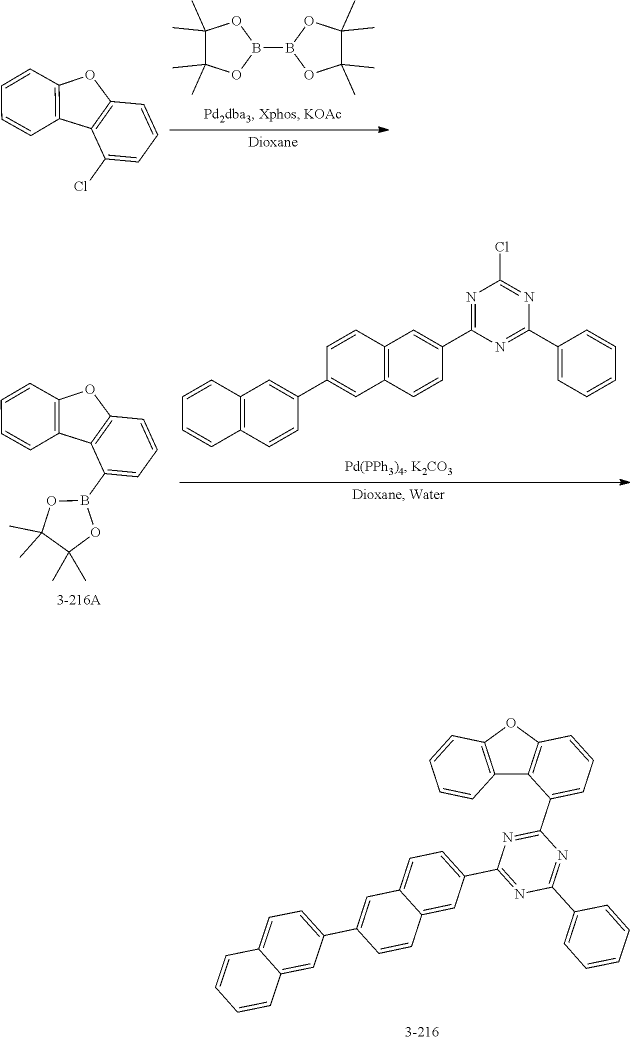

1-chlorodibenzo[b,d]furan (10 g, 49.5 mmol), bis(pinacolato)diboron (18.9 g, 74.3 mmol), Pd(PPh₃)₄ (2.9 g, 2.5 mmol) and KOAc (9.7 g, 99.0 mmol) were dissolved in 1,4-dioxane (100 mL), and then the mixture was refluxed for 3 hours.

After the reaction was completed, the reaction material was filtered under reduced pressure at room temperature, and then the solvent of the filtrate was removed using a rotary evaporator. After that, the result was dissolved in dichloromethane (DCM) and purified by silica, and then recrystallized with dichloromethane and methanol to obtain Compound 3-216A (12.4 g, yield 85%).

### Preparation Example 4-2. Preparation of Compound 3-216

Compound 3-216A (12.4 g, 42.1 mmol), 2-([2,2′-binaphthalen]-6-yl)-4-chloro-6-phenyl-1,3,5-triazine (18.7 g, 42.1 mmol), Pd₂(dba)₃ (1.9 g, 2.1 mmol), Xphos (2.0 g, 4.2 mmol) and K₂CO₃ (11.6 g, 84.2 mmol) were dissolved in 1,4-dioxane (120 mL) and distilled water (24 mL), and then the mixture was refluxed for 3 hours.

After the reaction was completed, the reaction material was filtered under reduced pressure at room temperature. After that, the result was dissolved in 1,2-dichlorobenzene (DCB) and purified by silica, and then recrystallized with dichloromethane and methanol to obtain target Compound 3-216 (18.9 g, yield 78%).

Synthesis results for the compounds described in Preparation Examples 3 and 4, and Table 9 are shown in the following Tables 10 and 11. The following Table 10 shows measurement values of ¹H NMR (CDCl₃, 400 MHz), and the following Table 11 shows measurement values of field desorption mass spectrometry (FD-MS).

TABLE 10

| Compound | |
|---|---|
| No. | ¹H NMR (CDCl₃, 400 MHz) |
| 3-59 | δ = 8.36(4H, s), 8.09(1H, d), 8.06(1H, d), 7.99(1H, d), 7.98(1H, d), 7.86(1H, d), 7.81(1H, d), |
| | 7.63(1H, d), 7.6(1H, d), 7.55(1H, dd), 7.54(1H, d), 7.5(6H, d), 7.39(1H, dd), 7.38(1H, dd), |
| | 7.31(1H, s), 7.25 (4H, t), |
| 3-62 | δ = 9.09(1H, s), 8.49(1H, d), 8.36(2H, d), 8.16(1H, d), 8.08(1H, d), 8(1H, d), 7.98(1H, d), |
| | 7.97(2H, d), 7.86(1H, d), 7.81(1H, dd), 7.75(2H, d), 7.61(3H, d), 7.59(1H, dd), 7.54(1H, dd), |
| | 7.5(3H, s), 7.49 (2H, t), 7.41 (1H, t), 7.4 (2H, dd), 7.39 (1H, dd), 7.31 (1H, t), |
| 3-97 | δ = 9.09(1H, s), 8.49(1H, d), 8.16(1H, d), 8.08(1H, d), 8(1H, d), 7.98(2H, d), 7.86(1H, d), |
| | 7.82(1H, d), 7.81(1H, d), 7.79(2H, dd), 7.69(1H, d), 7.61(1H, d), 7.59(1H, dd), 7.57(1H, dd), |
| | 7.54(2H, s), 7.46 (2H, t), 7.41 (1H, t), 7.39 (2H, dd), 7.31 (2H, dd), |
| 3-132 | δ = 9.09(1H, s), 8.49(1H, d), 8.36(2H, d), 8.16(1H, d), 8.09(1H, d), 8.08(1H, d), 8.06(1H, d), |
| | 8.03(1H, d), 8(1H, d), 7.99(1H, dd), 7.82(2H, d), 7.76(1H, d), 7.69(1H, dd), 7.63(1H, dd), |
| | 7.61(1H, s), 7.6 (1H, t), 7.59 (1H, t), 7.57 (1H, dd), 7.55 (1H, dd), 7.5 (3H, t), 7.38 (1H, d), |
| 3-216 | δ = 9.15(1H, s), 8.51(1H, d), 8.36(2H, d), 8.09(1H, d), 8.06(1H, d), 7.99(2H, d), 7.98(1H, d), |
| | 7.91(1H, d), 7.82(1H, d), 7.69(1H, dd), 7.63(1H, d), 7.6(1H, d), 7.57(1H, dd), 7.55(2H, dd), |
| | 7.54(1H, s), 7.5 (3H, t), 7.39 (1H, t), 7.38 (2H, dd), 7.31 (1H, dd), |

TABLE 11

| Compound | FD-Mass | Compound | FD-Mass |
|---|---|---|---|
| 3-59 | m/z = 601.22 (C₄₃H₂₇N₃O, 601.71) | 3-62 | m/z = 651.23 (C₄₇H₂₉N₃O, 651.77) |
| 3-97 | m/z = 651.23 (C₄₇H₂₉N₃O, 651.77) | 3-132 | m/z = 2.615 (C₄₃H₂₅N₃O₂, 615.69) |
| 3-216 | m/z = 2.615 (C₄₃H₂₅N₃O₂, 615.69) | | |

### Experimental Example 1

### Experimental Example 1-1. Manufacture of Organic Light Emitting Device

A glass substrate on which ITO was coated as a thin film to thicknesses of 1,500 Å was ultrasonic cleaned with distilled water. When the cleaning with distilled water was finished, the substrate was ultrasonic cleaned with solvents such as acetone, methanol and isopropyl alcohol, then dried, and then subjected to UVO (ultraviolet ozone) treatment for 5 minutes using UV (ultraviolet) in a UV cleaner. After that, the substrate was transferred to a plasma cleaner (PT), then subjected to plasma treatment under vacuum for ITO work function increase and residual film removal, and transferred to a thermal deposition apparatus for organic deposition.

On the transport ITO electrode (positive electrode), a hole injection layer 4,4′,4″-tris[2-naphthyl(phenyl)amino]triphenylamine (2-TNATA) and a hole transport layer N,N′-bis(α-naphthyl)-N,N′-diphenyl-4,4′-diamine (NPB) were deposited.

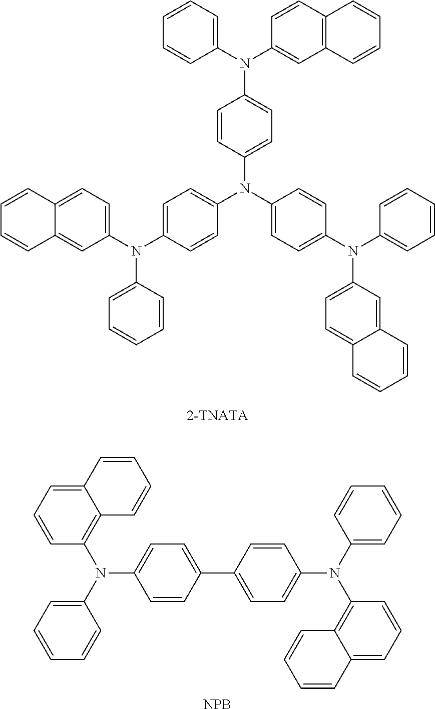 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; c1ccc(N(c2ccc(-c3ccc(N(c4ccccc4)c4cccc5ccccc45)cc3)cc2)c2cccc3ccccc23)cc1 ; "NPB"]

A light emitting layer was thermal vacuum deposited thereon as follows. The light emitting layer was deposited to a thickness of 500 Å using the heterocyclic compound represented by Chemical Formula 1 described in the following Table 12 as a red host and Ir(piq)₂(acac) as a red phosphorescent dopant, and by doping the red phosphorescent dopant to the host by 3%.

After that, BCP was deposited to a thickness of 60 Å as a hole blocking layer, and Alq₃ was deposited to a thickness of 200 Å thereon as an electron transport layer. Lastly, lithium fluoride (LiF) was deposited on the electron transport layer to a thickness of 10 Å to form an electron injection layer, and then aluminum (Al) was deposited on the electron injection layer to a thickness of 1,200 Å to form a negative electrode, and as a result, an organic electroluminescent device was manufactured.

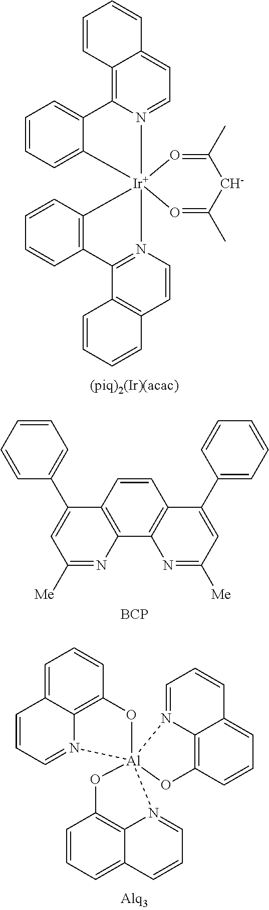

Meanwhile, all the organic compounds required to manufacture the OLED were vacuum sublimation purified under 10⁻⁸ torr to 10⁻⁶ torr for each material to be used in the manufacture of the OLED (organic light emitting device).

### Experimental Example 1-2. Driving Voltage, Light Emission Efficiency and Lifetime of Organic Light Emitting Device

For each of the organic light emitting devices manufactured as above, electroluminescent (EL) properties were measured using M7000 manufactured by McScience Inc., and with the measurement results, T₉₀ was measured when standard luminance was 6,000 cd/m² through a lifetime measurement system (M6000) manufactured by McScience Inc. Results of measuring driving voltage, light emission efficiency, color coordinate (CIE) and lifetime of the organic light emitting devices manufactured according to the present disclosure are shown in the following Table 12.

T₉₀ means a lifetime (unit: hour), a time taken for luminance to become 90% with respect to initial luminance.

TABLE 12

| | | | Driving | | | |
|---|---|---|---|---|---|---|
| | | Threshold | Voltage | Efficiency | Color Coordinate | Lifetime |
| | Compound | Voltage (Vₒₙ) | (Vₒₚ) | (cd/A) | (x, y) | (T₉₀) |
| Comparative | A | 2.63 | 5.53 | 5 | (0.683, 0.316) | 20 |
| Example 1 | | | | | | |
| Comparative | B | 2.70 | 5.22 | 15 | (0.681, 0.320) | 30 |
| Example 2 | | | | | | |
| Comparative | C | 2.65 | 5.45 | 20 | (0.683, 0.317) | 45 |
| Example 3 | | | | | | |
| Comparative | D | 2.74 | 5.54 | 25 | (0.682, 0.316) | 50 |
| Example 4 | | | | | | |
| Comparative | E | 2.76 | 5.26 | 30 | (0.680, 0.324) | 25 |
| Example 5 | | | | | | |
| Comparative | F | 2.86 | 5.46 | 10 | (0.683, 0.326) | 50 |
| Example 6 | | | | | | |
| Comparative | G | 2.66 | 5.06 | 25 | (0.682, 0.321) | 55 |
| Example7 | | | | | | |
| Comparative | H | 2.70 | 5.22 | 15 | (0.681, 0.320) | 30 |
| Example 8 | | | | | | |
| Comparative | I | 2.65 | 5.45 | 20 | (0.683, 0.317) | 45 |
| Example 9 | | | | | | |
| Comparative | J | 2.64 | 5.43 | 25 | (0.271, 0.671) | 10 |
| Example 10 | | | | | | |
| Example 1 | 1-1 | 2.40 | 4.90 | 73 | (0.681, 0.323) | 235 |
| Example 2 | 1-2 | 2.43 | 4.73 | 77 | (0.682, 0.326) | 230 |
| Example 3 | 1-3 | 2.45 | 5.05 | 74 | (0.680, 0.324) | 245 |
| Example 4 | 1-4 | 2.35 | 4.85 | 76 | (0.678, 0.317) | 225 |
| Example 5 | 1-5 | 2.38 | 4.68 | 69 | (0.679, 0.325) | 228 |
| Example 6 | 1-6 | 2.37 | 4.87 | 83 | (0.681, 0.325) | 223 |
| Example 7 | 1-7 | 2.43 | 4.73 | 74 | (0.677, 0.317) | 245 |
| Example 8 | 1-8 | 2.47 | 4.87 | 68 | (0.677, 0.317) | 220 |
| Example 9 | 1-9 | 2.45 | 4.85 | 66 | (0.679, 0.325) | 275 |
| Example 10 | 1-10 | 2.35 | 4.85 | 77 | (0.676, 0.323) | 225 |
| Example 11 | 1-11 | 2.47 | 4.97 | 71 | (0.679, 0.319) | 231 |
| Example 12 | 1-12 | 2.46 | 4.76 | 78 | (0.677, 0.324) | 223 |
| Example 13 | 1-13 | 2.43 | 4.73 | 66 | (0.680, 0.322) | 222 |
| Example 14 | 1-14 | 2.41 | 5.01 | 76 | (0.682, 0.316) | 224 |
| Example 15 | 1-15 | 2.32 | 4.72 | 71 | (0.678, 0.324) | 223 |
| Example 16 | 1-16 | 2.41 | 4.91 | 78 | (0.682, 0.322) | 234 |
| Example 17 | 1-17 | 2.42 | 5.12 | 68 | (0.679, 0.318) | 246 |
| Example 18 | 1-18 | 2.48 | 5.18 | 77 | (0.682, 0.326) | 238 |
| Example 19 | 1-19 | 2.38 | 5.08 | 72 | (0.677, 0.322) | 240 |
| Example 20 | 1-20 | 2.39 | 4.89 | 79 | (0.679, 0.326) | 228 |
| Example 21 | 1-21 | 2.45 | 4.75 | 69 | (0.677, 0.320) | 230 |
| Example 22 | 1-22 | 2.39 | 4.99 | 75 | (0.678, 0.324) | 231 |
| Example 23 | 1-23 | 2.43 | 5.13 | 74 | (0.681, 0.322) | 243 |
| Example 24 | 1-25 | 2.41 | 5.11 | 51 | (0.683, 0.316) | 285 |
| Example 25 | 1-28 | 2.32 | 4.62 | 56 | (0.676, 0.322) | 275 |
| Example 26 | 1-30 | 2.33 | 4.73 | 60 | (0.679, 0.322) | 270 |
| Example 27 | 1-37 | 2.4 | 4.90 | 55 | (0.681, 0.322) | 280 |
| Example 28 | 1-40 | 2.34 | 4.84 | 51 | (0.683, 0.326) | 283 |
| Example 29 | 1-44 | 2.43 | 4.73 | 52 | (0.680, 0.317) | 285 |
| Example 30 | 1-45 | 2.49 | 5.19 | 53 | (0.680, 0.324) | 290 |
| Example 31 | 1-49 | 2.41 | 4.91 | 37 | (0.682, 0.321) | 120 |
| Example 32 | 1-51 | 2.47 | 4.97 | 36 | (0.677, 0.316) | 100 |
| Example 33 | 1-55 | 2.48 | 4.88 | 41 | (0.676, 0.324) | 185 |
| Example 34 | 1-59 | 2.49 | 5.19 | 36 | (0.678, 0.321) | 130 |
| Example 35 | 1-65 | 2.39 | 5.09 | 39 | (0.676, 0.326) | 105 |
| Example 36 | 1-68 | 2.41 | 5.01 | 36 | (0.676, 0.316) | 110 |
| Example 37 | 1-73 | 2.55 | 4.95 | 53 | (0.679, 0.319) | 110 |
| Example 38 | 1-78 | 2.52 | 5.02 | 61 | (0.682, 0.321) | 100 |
| Example 39 | 1-88 | 2.53 | 5.03 | 52 | (0.680, 0.325) | 140 |
| Example 40 | 1-92 | 2.57 | 5.27 | 51 | (0.679, 0.316) | 120 |
| Example 41 | 1-94 | 2.54 | 4.84 | 50 | (0.677, 0.322) | 130 |
| Example 42 | 1-97 | 2.56 | 5.16 | 38 | (0.679, 0.325) | 235 |
| Example 43 | 1-100 | 2.58 | 4.98 | 42 | (0.680, 0.324) | 245 |
| Example 44 | 1-106 | 2.51 | 4.91 | 40 | (0.677, 0.317) | 230 |
| Example 45 | 1-111 | 2.57 | 5.07 | 41 | (0.680, 0.322) | 235 |
| Example 46 | 1-120 | 2.54 | 5.24 | 39 | (0.683, 0.318) | 225 |
| Example 47 | 1-121 | 2.57 | 5.27 | 36 | (0.682, 0.321) | 110 |
| Example 48 | 1-123 | 2.59 | 4.99 | 37 | (0.682, 0.325) | 150 |
| Example 49 | 1-132 | 2.55 | 5.05 | 38 | (0.680, 0.318) | 115 |
| Example 50 | 1-142 | 2.56 | 4.96 | 37 | (0.682, 0.317) | 140 |
| Example 51 | 1-145 | 2.40 | 4.70 | 72 | (0.683, 0.325) | 225 |
| Example 52 | 1-151 | 2.54 | 5.24 | 39 | (0.684, 0.315) | 250 |
| Example 53 | 1-157 | 2.42 | 4.95 | 74 | (0.681, 0.323) | 255 |
| Example 54 | 1-160 | 2.60 | 4.90 | 40 | (0.681, 0.317) | 200 |
| Example 55 | 2-1 | 2.39 | 4.69 | 73 | (0.681, 0.321) | 235 |
| Example 56 | 2-2 | 2.43 | 5.13 | 76 | (0.683, 0.324) | 230 |
| Example 57 | 2-3 | 2.45 | 5.05 | 74 | (0.680, 0.324) | 245 |
| Example 58 | 2-4 | 2.35 | 4.65 | 77 | (0.683, 0.319) | 225 |
| Example 59 | 2-5 | 2.34 | 4.64 | 73 | (0.680, 0.321) | 233 |
| Example 60 | 2-6 | 2.38 | 4.88 | 83 | (0.680, 0.323) | 220 |
| Example 61 | 2-7 | 2.49 | 5.19 | 78 | (0.681, 0.323) | 240 |
| Example 62 | 2-9 | 2.50 | 5.00 | 66 | (0.680, 0.321) | 270 |
| Example 63 | 2-10 | 2.31 | 4.91 | 73 | (0.679, 0.323) | 223 |
| Example 64 | 2-12 | 2.47 | 4.87 | 76 | (0.682, 0.320) | 220 |
| Example 65 | 2-13 | 2.42 | 5.02 | 69 | (0.683, 0.320) | 223 |
| Example 66 | 2-14 | 2.41 | 4.91 | 78 | (0.678, 0.324) | 240 |
| Example 67 | 2-15 | 2.32 | 4.92 | 74 | (0.682, 0.318) | 225 |
| Example 68 | 2-16 | 2.39 | 5.09 | 79 | (0.676, 0.322) | 240 |
| Example 69 | 2-17 | 2.38 | 4.68 | 71 | (0.676, 0.320) | 250 |
| Example 70 | 2-19 | 2.48 | 4.88 | 72 | (0.682, 0.323) | 230 |
| Example 71 | 2-20 | 2.41 | 4.81 | 78 | (0.679, 0.325) | 220 |
| Example 72 | 2-22 | 2.46 | 4.76 | 74 | (0.68, 0.324) | 230 |
| Example 73 | 2-23 | 2.43 | 5.13 | 72 | (0.677, 0.320) | 235 |
| Example 74 | 2-25 | 2.50 | 4.80 | 77 | (0.676, 0.318) | 230 |
| Example 75 | 2-28 | 2.45 | 4.85 | 71 | (0.681, 0.320) | 225 |
| Example 76 | 2-29 | 2.41 | 4.71 | 51 | (0.681, 0.320) | 285 |
| Example 77 | 2-31 | 2.49 | 4.99 | 52 | (0.677, 0.316) | 295 |
| Example 78 | 2-38 | 2.35 | 4.75 | 57 | (0.677, 0.326) | 275 |
| Example 79 | 2-46 | 2.48 | 5.18 | 58 | (0.683, 0.324) | 280 |
| Example 80 | 2-52 | 2.50 | 5.20 | 55 | (0.678, 0.319) | 270 |
| Example 81 | 2-57 | 2.42 | 5.12 | 37 | (0.678, 0.326) | 165 |
| Example 82 | 2-60 | 2.32 | 4.82 | 40 | (0.679, 0.317) | 100 |
| Example 83 | 2-68 | 2.49 | 4.79 | 41 | (0.683, 0.319) | 105 |
| Example 84 | 2-70 | 2.39 | 4.89 | 42 | (0.678, 0.319) | 120 |
| Example 85 | 2-74 | 2.47 | 4.97 | 40 | (0.682, 0.325) | 115 |
| Example 86 | 2-78 | 2.43 | 4.83 | 38 | (0.679, 0.316) | 150 |
| Example 87 | 2-85 | 2.54 | 5.04 | 52 | (0.677, 0.323) | 160 |
| Example 88 | 2-91 | 2.59 | 5.09 | 57 | (0.682, 0.317) | 195 |
| Example 89 | 2-100 | 2.53 | 5.03 | 58 | (0.679, 0.326) | 200 |
| Example 90 | 2-105 | 2.58 | 4.88 | 51 | (0.683, 0.316) | 160 |
| Example 91 | 2-108 | 2.60 | 5.30 | 55 | (0.681, 0.320) | 155 |
| Example 92 | 2-109 | 2.59 | 5.19 | 54 | (0.683, 0.317) | 145 |
| Example 93 | 2-113 | 2.55 | 5.25 | 36 | (0.682, 0.316) | 230 |
| Example 94 | 2-120 | 2.58 | 4.88 | 31 | (0.680, 0.324) | 220 |
| Example 95 | 2-126 | 2.54 | 4.84 | 39 | (0.683, 0.326) | 235 |
| Example 96 | 2-131 | 2.51 | 5.01 | 36 | (0.682, 0.321) | 230 |
| Example 97 | 2-134 | 2.54 | 5.14 | 35 | (0.681, 0.323) | 240 |
| Example 98 | 2-139 | 2.57 | 5.07 | 35 | (0.682, 0.326) | 235 |
| Example 99 | 2-141 | 2.55 | 5.15 | 37 | (0.680, 0.324) | 140 |
| Example 100 | 2-147 | 2.59 | 5.29 | 40 | (0.678, 0.317) | 170 |
| Example 101 | 2-160 | 2.56 | 4.86 | 42 | (0.679, 0.325) | 105 |
| Example 102 | 2-161 | 2.59 | 5.09 | 37 | (0.681, 0.325) | 220 |
| Example 103 | 2-178 | 2.56 | 5.06 | 53 | (0.677, 0.317) | 240 |

Comparative Example Compounds used in Table 12 are as follows.

### Comparative Example Compounds

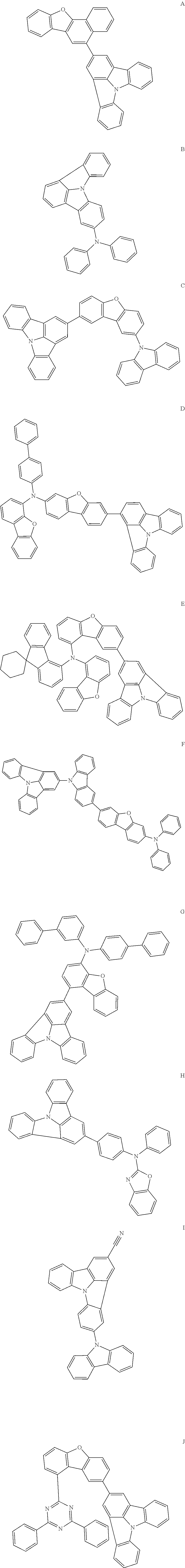

From the results of Table 12, it was seen that the organic light emitting device including the heterocyclic compound represented by Chemical Formula 1 of the present disclosure in the organic material layer had excellent light emission efficiency and lifetime by having an amine present at the No. 7 position, thereby increasing processability and controlling mobility. In addition, by having indolo[3,2,1-jk]carbazole present at the No. 5 position, crystallinity becomes superior, resulting in a property of lowering driving voltage due to improved conductivity, and each performance may be controlled since conductivity is controlled by having it present at each position.

In other words, the heterocyclic compound represented by Chemical Formula 1 of the present disclosure has excellent crystallinity due to indolo[3,2,1-jk]carbazole, and by having balanced electrons and holes, results of low driving voltage, and excellent light emission efficiency and lifetime were obtained.

On the other hand, the organic light emitting device including the Comparative Example Compound in the organic material layer had a higher driving voltage compared to the Examples, and had lower light emission efficiency and lifetime compared to the Examples.

Comparative Example Compounds A to J have indolo[3,2,1-jk]carbazole as a core, and have one substituent. Compounds A to C of Comparative Examples 1 to 3 are small in size and interrupt the flow of electrons, resulting in unbalanced electrons and holes, and accordingly, results of high driving voltage and low lifetime properties were obtained. Compound D of Comparative Example 4 has a different position of substitution from Chemical Formula 1 of the present disclosure. Due to such linear position of substitution, a deep HOMO (highest occupied molecular orbital) level was obtained, which blocks holes, and a result of reducing a lifetime was obtained. Compound E of Comparative Example 5 has steric hindrance due to a large substituent, lowering crystallinity and thereby lowering mobility, and as a result, poor performance was obtained due to low electron mobility. Compound F of Comparative Example 6 has a different position of substitution from Chemical Formula 1 of the present disclosure. In addition, carbazole is connected as a linker, strengthening an electron donating region, however, electrons and holes are not balanced, resulting in poor performance. Compound G of Comparative Example 7 has an unstable structure due to steric hindrance by the substitution position of terphenyl group, and accordingly, a result of low lifetime properties was obtained. Compound H of Comparative Example 8 and Compound I of Comparative Example 9 are small in size, have high crystallinity and high charge mobility, however, electrons and holes are not balanced, resulting in poor performance. Compound J of Comparative Example 10 has excellent electron injection and transport abilities and excellent light emission efficiency, but has a small energy band gap, which is suitable for a green host, and a result of very low lifetime was obtained. In addition, Compound J is a compound suitable for an n-host, having an application different from the heterocyclic compound represented by Chemical Formula 1 of the present disclosure, and a result of poor lifetime properties was obtained.

Accordingly, the heterocyclic compound represented by Chemical Formula 1 of the present disclosure may have lower driving voltage, higher light emission efficiency and longer lifetime compared to the compounds of Comparative Examples.

### Experimental Example 2

### Experimental Example 2-1. Manufacture of Organic Light Emitting Device

An organic light emitting device was manufactured in the same manner as in Experimental Example 1-1, except that, as the host of the light emitting layer, one type of a first host (heterocyclic compound represented by Chemical Formula 1, P type) and one type of a second host (heterocyclic compound represented by Chemical Formula 2, N type) described in the following Table 13 were pre-mixed and then deposited in one source of supply.

### Experimental Example 2-2. Driving Voltage and Light Emission Efficiency of Organic Light Emitting Device

For each of the organic light emitting devices manufactured as above, electroluminescent (EL) properties were measured using M7000 manufactured by McScience Inc., and with the measurement results, T₉₀ was measured when standard luminance was 6,000 cd/m² through a lifetime measurement system (M6000) manufactured by McScience Inc. Results of measuring driving voltage, light emission efficiency, color coordinate (CIE) and lifetime of the organic light emitting devices manufactured according to the present disclosure are shown in the following Table 13.

T₉₀ means a lifetime (unit: hour), a time taken for luminance to become 90% with respect to initial luminance.

TABLE 13

| | | | | Thresh | | | | |
|---|---|---|---|---|---|---|---|---|
| | | | | old | Driving | | Color | |
| | Compound | | Ratio | Voltage | Voltage | Efficiency | Coordinate | Lifetime |
| | P | N | (P:N) | (Vₒₙ) | (Vₒₚ) | (cd/A) | (x, y) | (T₉₀) |
| Comparative | A | 3-59 | 1:1 | 2.58 | 5.13 | 18.86 | (0.677, 0.321) | 40 |
| Example 11 | | | | | | | | |
| Comparative | B | 3-97 | 1:1 | 2.64 | 4.86 | 32.14 | (0.682, 0.316) | 50 |
| Example 12 | | | | | | | | |
| Comparative | D | 3-132 | 1:1 | 2.68 | 5.06 | 38.97 | (0.677, 0.326) | 80 |
| Example 13 | | | | | | | | |
| Comparative | G | 3-216 | 1:1 | 2.59 | 4.68 | 41.56 | (0.678, 0.326) | 100 |
| Example 14 | | | | | | | | |
| Comparative | J | 3-59 | 1:1 | 2.78 | 5.25 | 15.75 | (0.672, 0.370) | 25 |
| Example 15 | | | | | | | | |
| Example 104 | 1-25 | 3-59 | 1:1 | 2.32 | 4.22 | 67.78 | (0.679, 0.323) | 750 |
| Example 105 | 1-37 | 3-59 | 1:1 | 2.33 | 4.23 | 70.26 | (0.683, 0.322) | 700 |
| Example 106 | 1-6 | 3-59 | 1:1 | 2.28 | 4.18 | 100.08 | (0.680, 0.325) | 500 |
| Example 107 | 1-121 | 3-59 | 1:1 | 2.48 | 4.38 | 50.68 | (0.678, 0.326) | 320 |
| Example 108 | 1-145 | 3-59 | 1:1 | 2.31 | 4.41 | 86.2 | (0.676, 0.320) | 400 |
| Example 109 | 1-44 | 3-62 | 1:1 | 2.34 | 4.24 | 68.59 | (0.680, 0.316) | 690 |
| Example 110 | 1-12 | 3-62 | 1:1 | 2.38 | 4.38 | 95.11 | (0.680, 0.316) | 480 |
| Example 111 | 1-16 | 3-62 | 1:1 | 2.34 | 4.24 | 94.7 | (0.681, 0.316) | 600 |
| Example 112 | 1-132 | 3-62 | 1:1 | 2.47 | 4.37 | 52.64 | (0.680, 0.318) | 300 |
| Example 113 | 1-45 | 3-97 | 1:1 | 2.40 | 4.3 | 68.68 | (0.679, 0.323) | 700 |
| Example 114 | 2-6 | 3-97 | 1:1 | 2.29 | 4.29 | 99.45 | (0.678, 0.322) | 530 |
| Example 115 | 2-141 | 3-97 | 1:1 | 2.48 | 4.38 | 52.11 | (0.681, 0.318) | 250 |
| Example 116 | 1-160 | 3-97 | 1:1 | 2.54 | 4.54 | 56.77 | (0.679, 0.319) | 350 |
| Example 117 | 2-29 | 3-132 | 1:1 | 2.32 | 4.22 | 68.02 | (0.681, 0.324) | 700 |
| Example 118 | 2-7 | 3-132 | 1:1 | 2.42 | 4.52 | 94.96 | (0.681, 0.316) | 830 |
| Example 119 | 2-160 | 3-132 | 1:1 | 2.47 | 4.37 | 56.88 | (0.678, 0.321) | 300 |
| Example 120 | 2-161 | 3-132 | 1:1 | 2.54 | 4.44 | 52.86 | (0.677, 0.324) | 350 |
| Example 121 | 2-31 | 3-216 | 1:1 | 2.39 | 4.29 | 67.96 | (0.682, 0.325) | 800 |
| Example 122 | 2-14 | 3-216 | 1:1 | 2.36 | 4.36 | 92.24 | (0.683, 0.316) | 650 |
| Example 123 | 2-16 | 3-216 | 1:1 | 2.32 | 4.42 | 94.15 | (0.679, 0.320) | 680 |
| Example 124 | 2-178 | 3-216 | 1:1 | 2.51 | 4.41 | 69.66 | (0.682, 0.317) | 300 |

Comparative Example Compounds used in Table 13 are as follows.

### Comparative Example Compounds

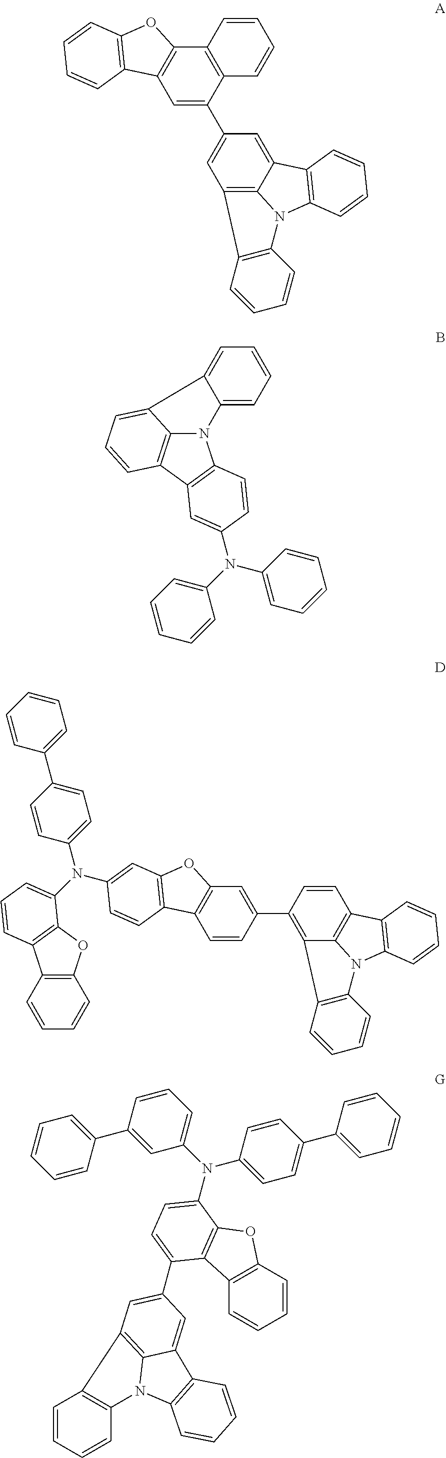

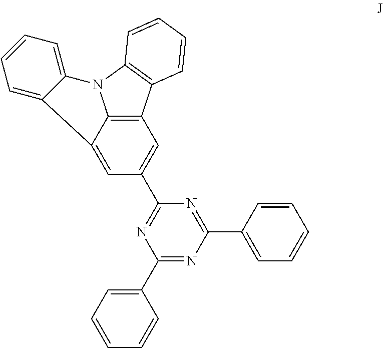 [SMILES: c1ccc(-c2nc(-c3ccccc3)nc(-c3cc4c5ccccc5n5c6ccccc6c(c3)c45)n2)cc1]

Comparing the results of Table 13 with the results of Table 12, it may be identified that driving voltage, light emission efficiency and lifetime are all improved when the heterocyclic compound represented by Chemical Formula 1 and the heterocyclic compound represented by Chemical Formula 2 of the present disclosure are used at the same time as a host of a light emitting layer.

In other words, it was able to be identified that excellent device properties were obtained when, as a host of a light emitting layer, the heterocyclic compound represented by Chemical Formula 1 was used for a donor role and the heterocyclic compound represented by Chemical Formula 2 was used for an acceptor role in the present disclosure.

On the other hand, it may be seen that, when the Comparative Example Compound and the heterocyclic compound represented by Chemical Formula 2 are used at the same time, performance in terms of driving voltage, light emission efficiency and lifetime declines compared to in the Examples.

Accordingly, it was able to be identified that driving voltage was low, and light emission efficiency and lifetime were significantly superior when the heterocyclic compound represented by Chemical Formula 1 and the heterocyclic compound represented by Chemical Formula 2 of the present disclosure are used at the same time as a host of a light emitting layer, and it was able to be identified that a larger lifetime improvement effect was obtained when the compounds are substituted with deuterium.

### REFERENCE NUMERAL

100: Substrate
200: Positive Electrode
300: Organic Material Layer
301: Hole Injection Layer
302: Hole Transport Layer
303: Light Emitting Layer
304: Hole Blocking Layer
305: Electron Transport Layer
306: Electron Injection Layer
400: Negative Electrode

1. A heterocyclic compound represented by the following Chemical Formula 1: 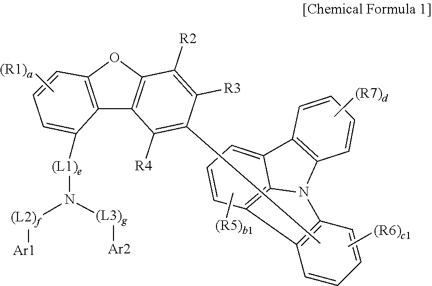
wherein, in Chemical Formula 1,
R1 to R7 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and —NR101R102, or two or more groups adjacent to each other bond to each other to form a substituted or unsubstituted C6 to C60 aromatic hydrocarbon ring; or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group;
a is an integer of 0 to 3, and when a is 2 or greater, R1s are the same as or different from each other;
b1 is an integer of 0 to 3, and when b1 is 2 or greater, R5s are the same as or different from each other;
c1 is an integer of 0 to 4, and when c1 is 2 or greater, R6s are the same as or different from each other;
d is an integer of 0 to 4, and when d is 2 or greater, R7s are the same as or different from each other;
L1 to L3 are the same as or different from each other, and each independently a direct bond; a substituted or unsubstituted C6 to C60 arylene group; or a substituted or unsubstituted C2 to C60 heteroarylene group;
e is an integer of 0 to 5, and when e is 2 or greater, L1s are the same as or different from each other;
f is an integer of 0 to 5, and when f is 2 or greater, L2s are the same as or different from each other;
g is an integer of 0 to 5, and when g is 2 or greater, L3s are the same as or different from each other; and
Ar1 and Ar2 are the same as or different from each other, and each independently a substituted or unsubstituted C6 to C60 aryl group; or a substituted or unsubstituted C2 to C60 heteroaryl group.

2. The heterocyclic compound of claim 1, wherein the heterocyclic compound represented by Chemical Formula 1 is represented by the following Chemical Formula 1-1 or 1-2: 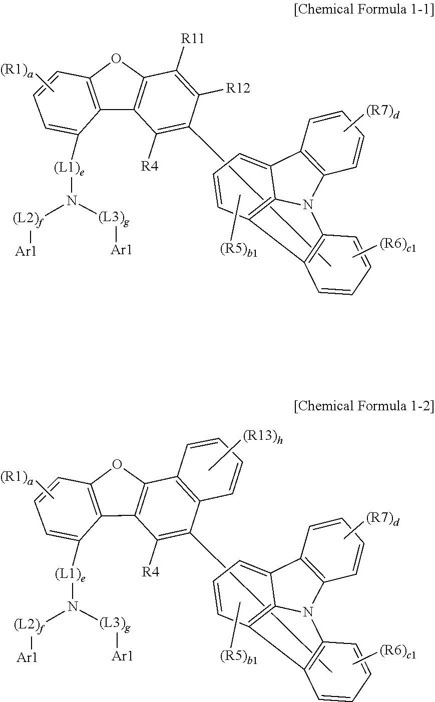
in Chemical Formulae 1-1 and 1-2,
R11 to R13 are the same as or different from each other and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and —NR101R102, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group;
h is an integer of 0 to 4, and when his 2 or greater, R13s are the same as or different from each other; and
R1, R4 to R7, L1 to L3, Ar1, Ar2, a, b1, c1 and d to g have the same definitions as in Chemical Formula 1.

3. The heterocyclic compound of claim 2, wherein the heterocyclic compound represented by Chemical Formula 1-1 is represented by the following Chemical Formula 1-1-1 or 1-1-2: 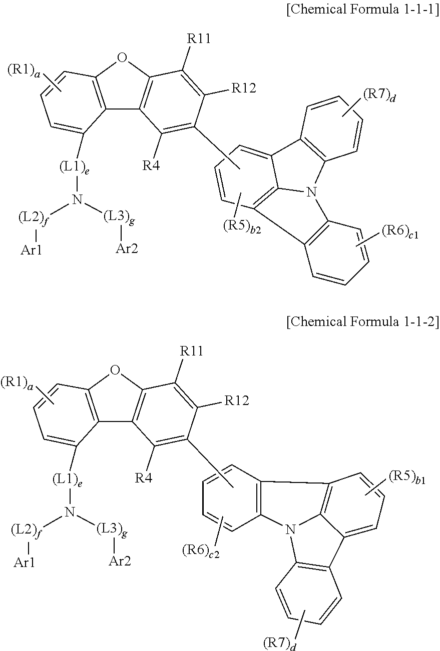
in Chemical Formulae 1-1-1 and 1-1-2,
b2 is an integer of 0 to 2, and when b2 is 2 or greater, R5s are the same as or different from each other;
c2 is an integer of 0 to 3, and when c2 is 2 or greater, R6s are the same as or different from each other;
R1, R4 to R7, L1 to L3, Ar1, Ar2, a, b1, c1 and d to g have the same definitions as in Chemical Formula 1; and
R11 and R12 have the same definitions as in Chemical Formula 1-1.

4. The heterocyclic compound of claim 2, wherein the heterocyclic compound represented by Chemical Formula 1-2 is represented by the following Chemical Formula 1-2-1 or 1-2-2: 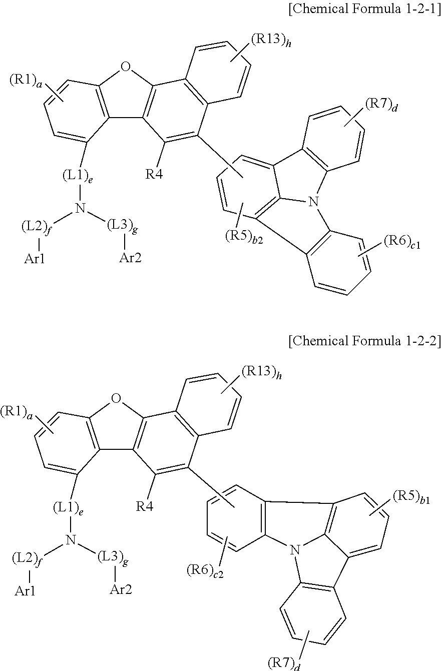
in Chemical Formulae 1-2-1 and 1-2-2,
b2 is an integer of 0 to 2, and when b2 is 2 or greater, R5s are the same as or different from each other;
c2 is an integer of 0 to 3, and when c2 is 2 or greater, R6s are the same as or different from each other;
R1, R4 to R7, L1 to L3, Ar1, Ar2, a, b1, c1 and d to f have the same definitions as in Chemical Formula 1; and
R13 and h have the same definitions as in Chemical Formula 1-2.

5. The heterocyclic compound of claim 1, wherein the heterocyclic compound represented by Chemical Formula 1 does not include deuterium as a substituent, or has a deuterium content of 1% to 100% with respect to a total number of hydrogen atoms and deuterium atoms.

6. The heterocyclic compound of claim 1, wherein the heterocyclic compound represented by Chemical Formula 1 is represented by any one of the following compounds:

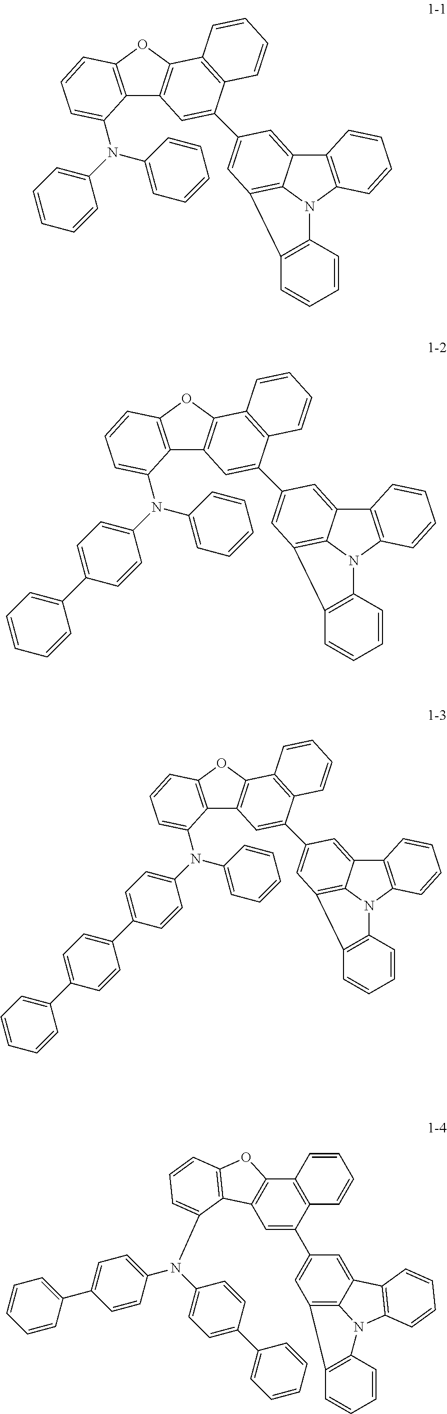 [SMILES: c1ccc(N(c2ccccc2)c2cccc3oc4c5ccccc5c(-c5cc6c7ccccc7n7c8ccccc8c(c5)c67)cc4c23)cc1 ; c1ccc(-c2ccc(N(c3ccccc3)c3cccc4oc5c6ccccc6c(-c6cc7c8ccccc8n8c9ccccc9c(c6)c78)cc5c34)cc2)cc1 ; c1ccc(-c2ccc(-c3ccc(N(c4ccccc4)c4cccc5oc6c7ccccc7c(-c7cc8c9ccccc9n9c%10ccccc%10c(c7)c89)cc6c45)cc3)cc2)cc1 ; c1ccc(-c2ccc(N(c3ccc(-c4ccccc4)cc3)c3cccc4oc5c6ccccc6c(-c6cc7c8ccccc8n8c9ccccc9c(c6)c78)cc5c34)cc2)cc1]

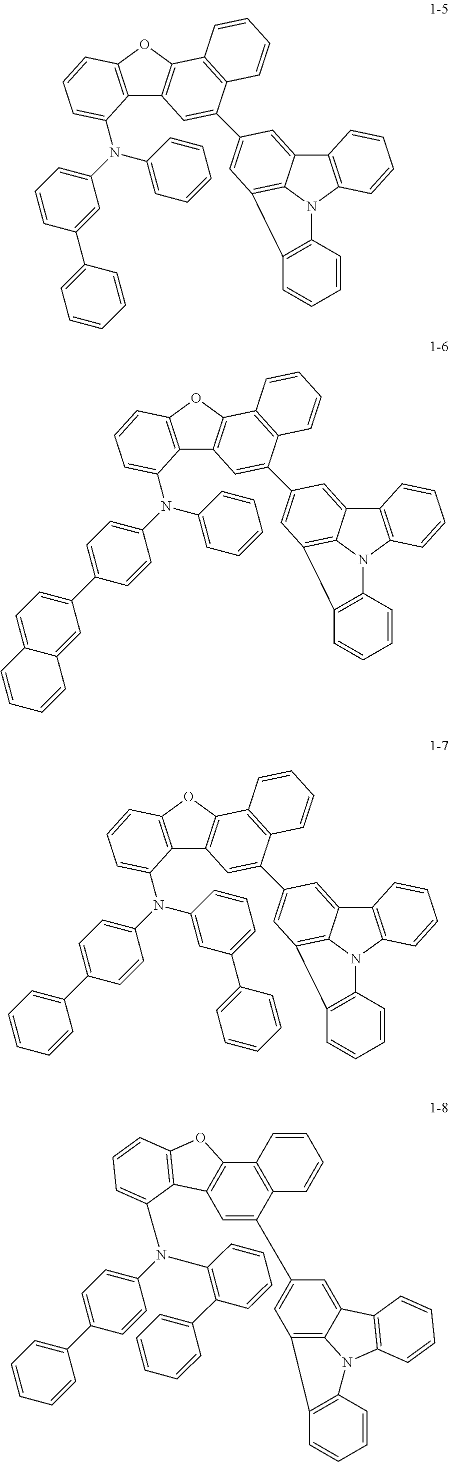 [SMILES: c1ccc(-c2cccc(N(c3ccccc3)c3cccc4oc5c6ccccc6c(-c6cc7c8ccccc8n8c9ccccc9c(c6)c78)cc5c34)c2)cc1 ; c1ccc(N(c2ccc(-c3ccc4ccccc4c3)cc2)c2cccc3oc4c5ccccc5c(-c5cc6c7ccccc7n7c8ccccc8c(c5)c67)cc4c23)cc1 ; c1ccc(-c2ccc(N(c3cccc(-c4ccccc4)c3)c3cccc4oc5c6ccccc6c(-c6cc7c8ccccc8n8c9ccccc9c(c6)c78)cc5c34)cc2)cc1 ; c1ccc(-c2ccc(N(c3ccccc3-c3ccccc3)c3cccc4oc5c6ccccc6c(-c6cc7c8ccccc8n8c9ccccc9c(c6)c78)cc5c34)cc2)cc1]

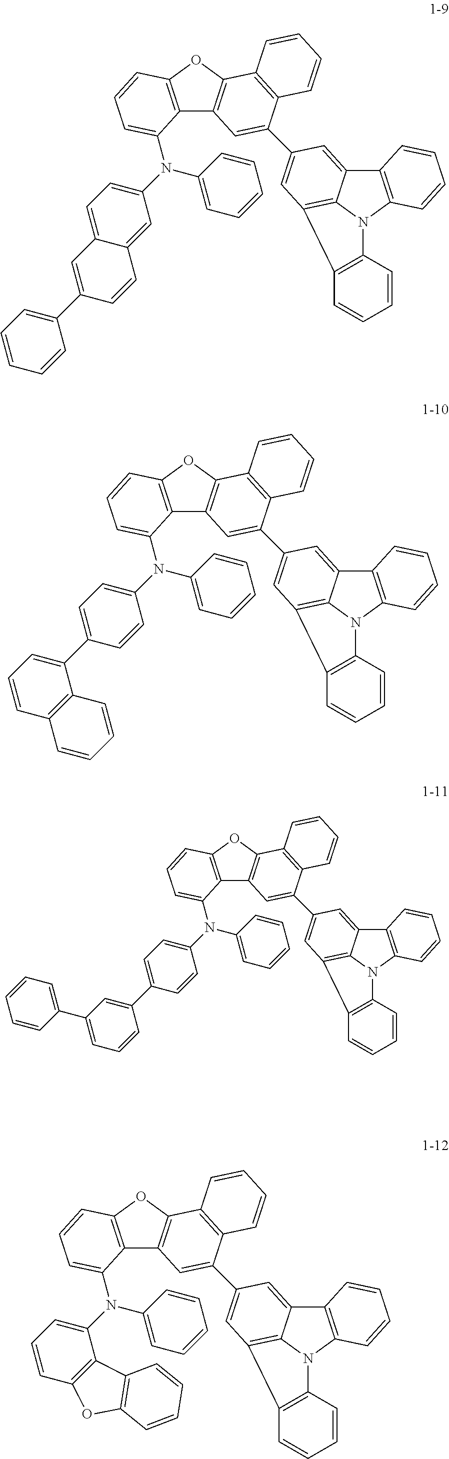 [SMILES: c1ccc(-c2ccc3cc(N(c4ccccc4)c4cccc5oc6c7ccccc7c(-c7cc8c9ccccc9n9c%10ccccc%10c(c7)c89)cc6c45)ccc3c2)cc1 ; c1ccc(N(c2ccc(-c3cccc4ccccc34)cc2)c2cccc3oc4c5ccccc5c(-c5cc6c7ccccc7n7c8ccccc8c(c5)c67)cc4c23)cc1 ; c1ccc(-c2cccc(-c3ccc(N(c4ccccc4)c4cccc5oc6c7ccccc7c(-c7cc8c9ccccc9n9c%10ccccc%10c(c7)c89)cc6c45)cc3)c2)cc1 ; c1ccc(N(c2cccc3oc4ccccc4c23)c2cccc3oc4c5ccccc5c(-c5cc6c7ccccc7n7c8ccccc8c(c5)c67)cc4c23)cc1]

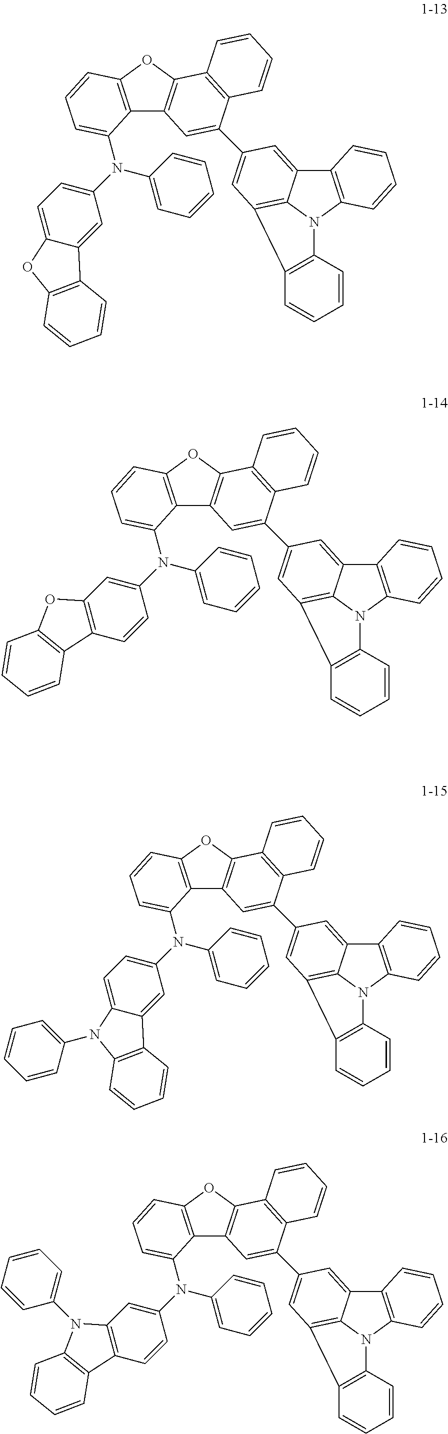 [SMILES: c1ccc(N(c2ccc3oc4ccccc4c3c2)c2cccc3oc4c5ccccc5c(-c5cc6c7ccccc7n7c8ccccc8c(c5)c67)cc4c23)cc1 ; c1ccc(N(c2ccc3c(c2)oc2ccccc23)c2cccc3oc4c5ccccc5c(-c5cc6c7ccccc7n7c8ccccc8c(c5)c67)cc4c23)cc1 ; c1ccc(N(c2ccc3c(c2)c2ccccc2n3-c2ccccc2)c2cccc3oc4c5ccccc5c(-c5cc6c7ccccc7n7c8ccccc8c(c5)c67)cc4c23)cc1 ; c1ccc(N(c2ccc3c4ccccc4n(-c4ccccc4)c3c2)c2cccc3oc4c5ccccc5c(-c5cc6c7ccccc7n7c8ccccc8c(c5)c67)cc4c23)cc1]

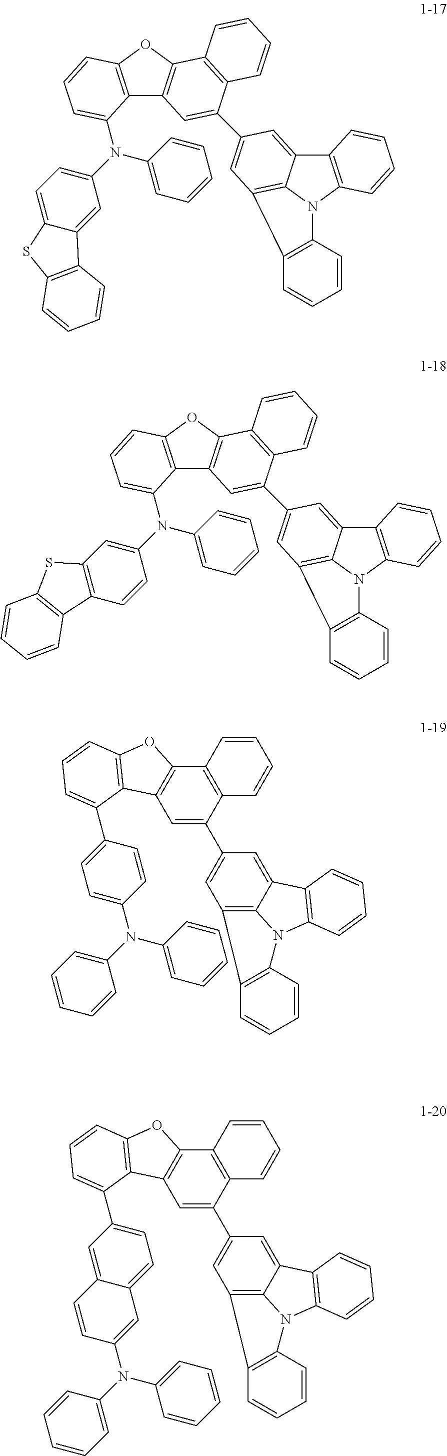 [SMILES: c1ccc(N(c2ccc3sc4ccccc4c3c2)c2cccc3oc4c5ccccc5c(-c5cc6c7ccccc7n7c8ccccc8c(c5)c67)cc4c23)cc1 ; c1ccc(N(c2ccc3c(c2)sc2ccccc23)c2cccc3oc4c5ccccc5c(-c5cc6c7ccccc7n7c8ccccc8c(c5)c67)cc4c23)cc1 ; c1ccc(N(c2ccccc2)c2ccc(-c3cccc4oc5c6ccccc6c(-c6cc7c8ccccc8n8c9ccccc9c(c6)c78)cc5c34)cc2)cc1 ; c1ccc(N(c2ccccc2)c2ccc3cc(-c4cccc5oc6c7ccccc7c(-c7cc8c9ccccc9n9c%10ccccc%10c(c7)c89)cc6c45)ccc3c2)cc1]

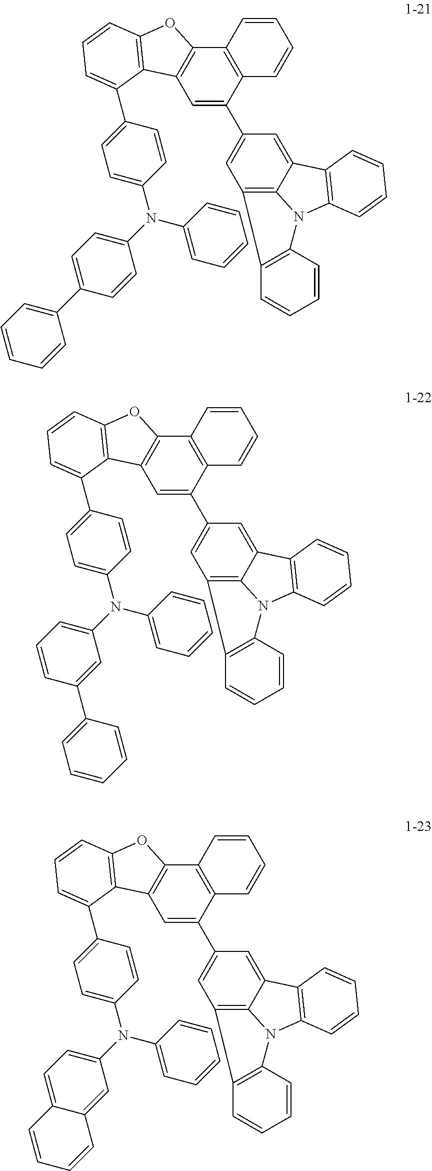 [SMILES: c1ccc(-c2ccc(N(c3ccccc3)c3ccc(-c4cccc5oc6c7ccccc7c(-c7cc8c9ccccc9n9c%10ccccc%10c(c7)c89)cc6c45)cc3)cc2)cc1 ; c1ccc(-c2cccc(N(c3ccccc3)c3ccc(-c4cccc5oc6c7ccccc7c(-c7cc8c9ccccc9n9c%10ccccc%10c(c7)c89)cc6c45)cc3)c2)cc1 ; c1ccc(N(c2ccc(-c3cccc4oc5c6ccccc6c(-c6cc7c8ccccc8n8c9ccccc9c(c6)c78)cc5c34)cc2)c2ccc3ccccc3c2)cc1]

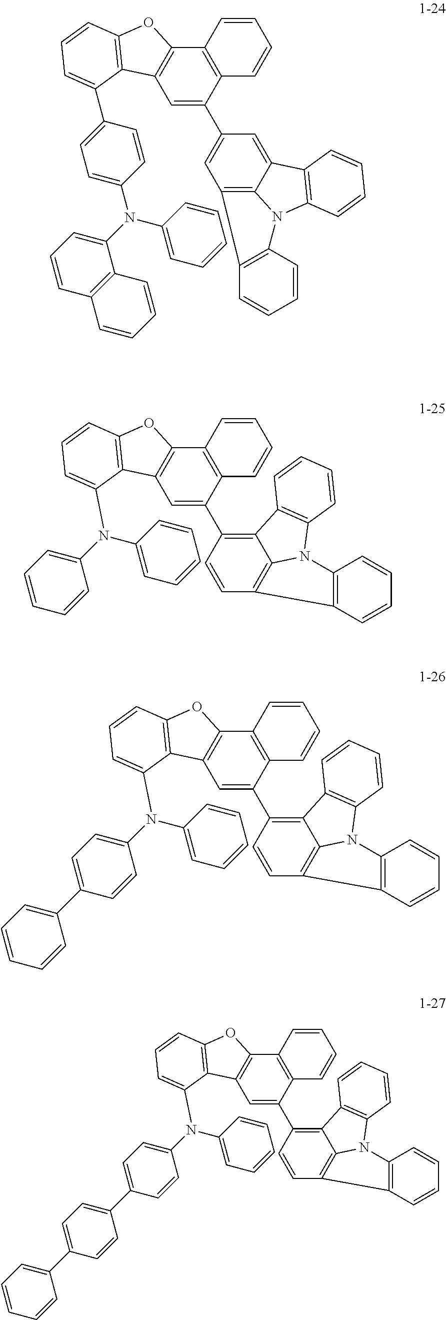 [SMILES: c1ccc(N(c2ccc(-c3cccc4oc5c6ccccc6c(-c6cc7c8ccccc8n8c9ccccc9c(c6)c78)cc5c34)cc2)c2cccc3ccccc23)cc1 ; c1ccc(N(c2ccccc2)c2cccc3oc4c5ccccc5c(-c5ccc6c7ccccc7n7c8ccccc8c5c67)cc4c23)cc1 ; c1ccc(-c2ccc(N(c3ccccc3)c3cccc4oc5c6ccccc6c(-c6ccc7c8ccccc8n8c9ccccc9c6c78)cc5c34)cc2)cc1 ; c1ccc(-c2ccc(-c3ccc(N(c4ccccc4)c4cccc5oc6c7ccccc7c(-c7ccc8c9ccccc9n9c%10ccccc%10c7c89)cc6c45)cc3)cc2)cc1]

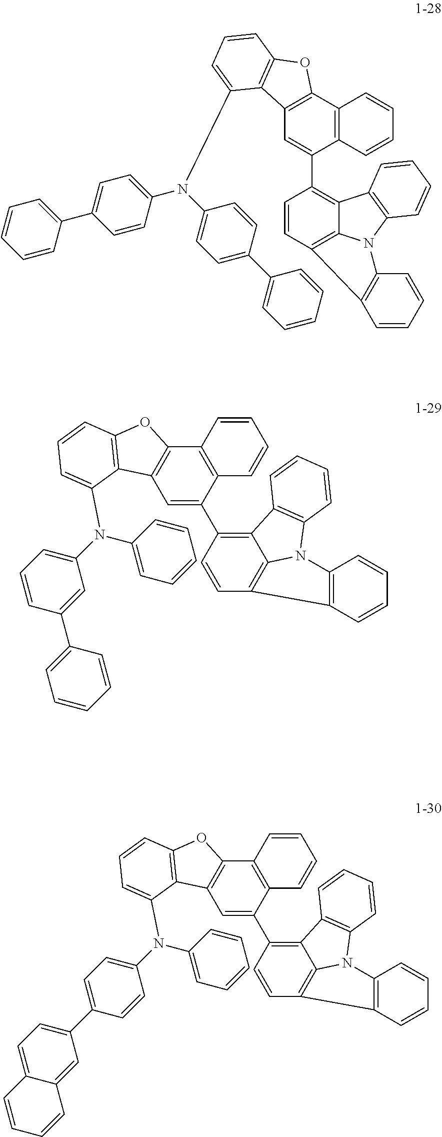 [SMILES: c1ccc(-c2ccc(N(c3ccc(-c4ccccc4)cc3)c3cccc4oc5c6ccccc6c(-c6ccc7c8ccccc8n8c9ccccc9c6c78)cc5c34)cc2)cc1 ; c1ccc(-c2cccc(N(c3ccccc3)c3cccc4oc5c6ccccc6c(-c6ccc7c8ccccc8n8c9ccccc9c6c78)cc5c34)c2)cc1 ; c1ccc(N(c2ccc(-c3ccc4ccccc4c3)cc2)c2cccc3oc4c5ccccc5c(-c5ccc6c7ccccc7n7c8ccccc8c5c67)cc4c23)cc1]

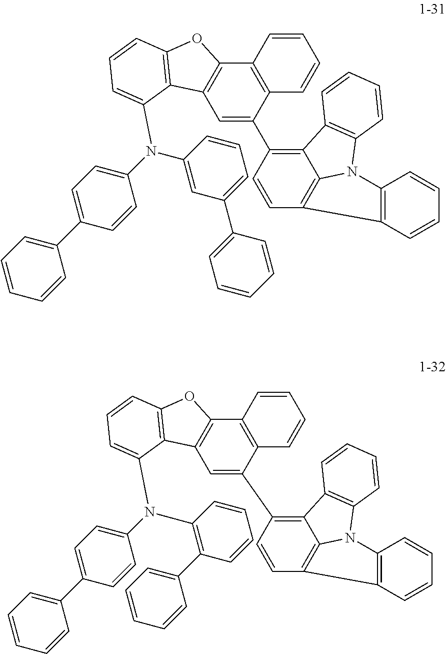 [SMILES: c1ccc(-c2ccc(N(c3cccc(-c4ccccc4)c3)c3cccc4oc5c6ccccc6c(-c6ccc7c8ccccc8n8c9ccccc9c6c78)cc5c34)cc2)cc1 ; c1ccc(-c2ccc(N(c3ccccc3-c3ccccc3)c3cccc4oc5c6ccccc6c(-c6ccc7c8ccccc8n8c9ccccc9c6c78)cc5c34)cc2)cc1]

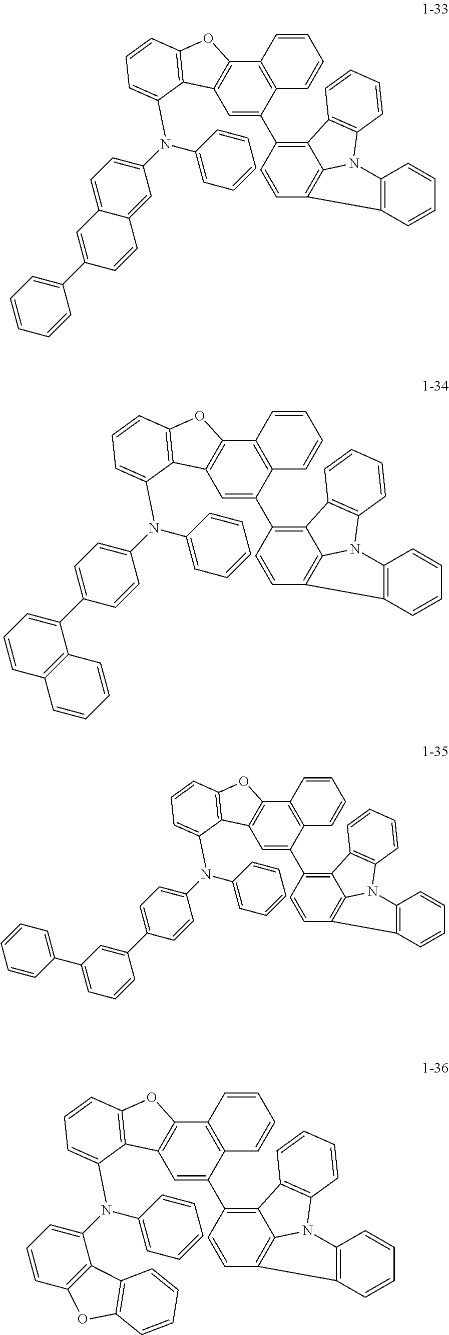 [SMILES: c1ccc(-c2ccc3cc(N(c4ccccc4)c4cccc5oc6c7ccccc7c(-c7ccc8c9ccccc9n9c%10ccccc%10c7c89)cc6c45)ccc3c2)cc1 ; c1ccc(N(c2ccc(-c3cccc4ccccc34)cc2)c2cccc3oc4c5ccccc5c(-c5ccc6c7ccccc7n7c8ccccc8c5c67)cc4c23)cc1 ; c1ccc(-c2cccc(-c3ccc(N(c4ccccc4)c4cccc5oc6c7ccccc7c(-c7ccc8c9ccccc9n9c%10ccccc%10c7c89)cc6c45)cc3)c2)cc1 ; c1ccc(N(c2cccc3oc4ccccc4c23)c2cccc3oc4c5ccccc5c(-c5ccc6c7ccccc7n7c8ccccc8c5c67)cc4c23)cc1]

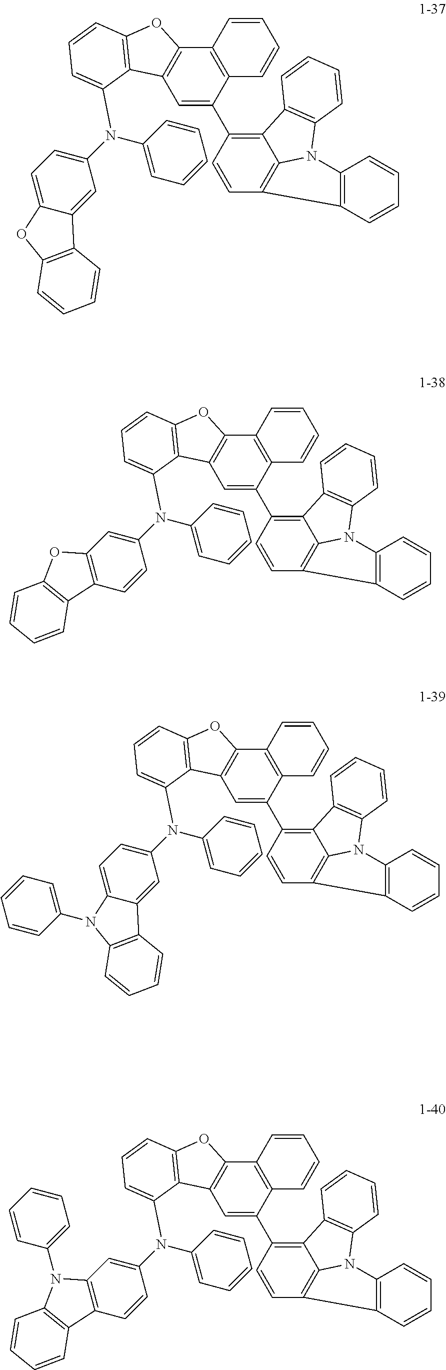 [SMILES: c1ccc(N(c2ccc3oc4ccccc4c3c2)c2cccc3oc4c5ccccc5c(-c5ccc6c7ccccc7n7c8ccccc8c5c67)cc4c23)cc1 ; c1ccc(N(c2ccc3c(c2)oc2ccccc23)c2cccc3oc4c5ccccc5c(-c5ccc6c7ccccc7n7c8ccccc8c5c67)cc4c23)cc1 ; c1ccc(N(c2ccc3c(c2)c2ccccc2n3-c2ccccc2)c2cccc3oc4c5ccccc5c(-c5ccc6c7ccccc7n7c8ccccc8c5c67)cc4c23)cc1 ; c1ccc(N(c2ccc3c4ccccc4n(-c4ccccc4)c3c2)c2cccc3oc4c5ccccc5c(-c5ccc6c7ccccc7n7c8ccccc8c5c67)cc4c23)cc1]

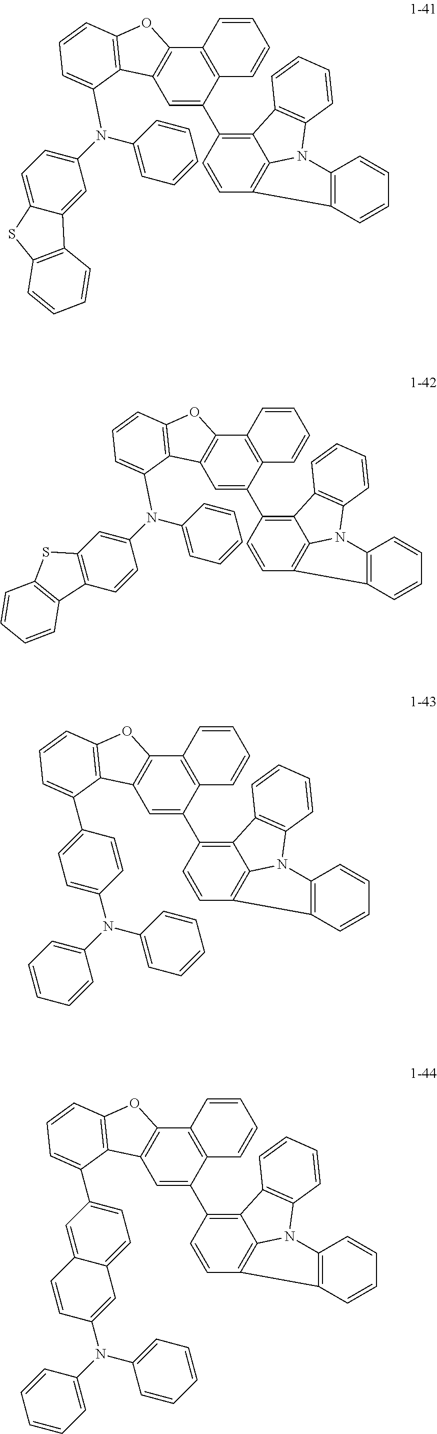 [SMILES: c1ccc(N(c2ccc3sc4ccccc4c3c2)c2cccc3oc4c5ccccc5c(-c5ccc6c7ccccc7n7c8ccccc8c5c67)cc4c23)cc1 ; c1ccc(N(c2ccc3c(c2)sc2ccccc23)c2cccc3oc4c5ccccc5c(-c5ccc6c7ccccc7n7c8ccccc8c5c67)cc4c23)cc1 ; c1ccc(N(c2ccccc2)c2ccc(-c3cccc4oc5c6ccccc6c(-c6ccc7c8ccccc8n8c9ccccc9c6c78)cc5c34)cc2)cc1 ; c1ccc(N(c2ccccc2)c2ccc3cc(-c4cccc5oc6c7ccccc7c(-c7ccc8c9ccccc9n9c%10ccccc%10c7c89)cc6c45)ccc3c2)cc1]

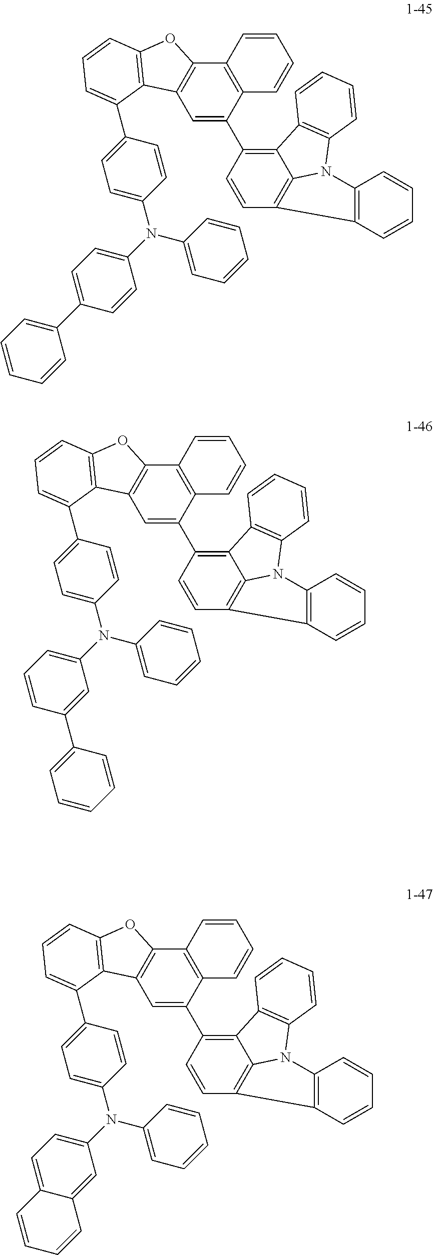 [SMILES: c1ccc(-c2ccc(N(c3ccccc3)c3ccc(-c4cccc5oc6c7ccccc7c(-c7ccc8c9ccccc9n9c%10ccccc%10c7c89)cc6c45)cc3)cc2)cc1 ; c1ccc(-c2cccc(N(c3ccccc3)c3ccc(-c4cccc5oc6c7ccccc7c(-c7ccc8c9ccccc9n9c%10ccccc%10c7c89)cc6c45)cc3)c2)cc1 ; c1ccc(N(c2ccc(-c3cccc4oc5c6ccccc6c(-c6ccc7c8ccccc8n8c9ccccc9c6c78)cc5c34)cc2)c2ccc3ccccc3c2)cc1]

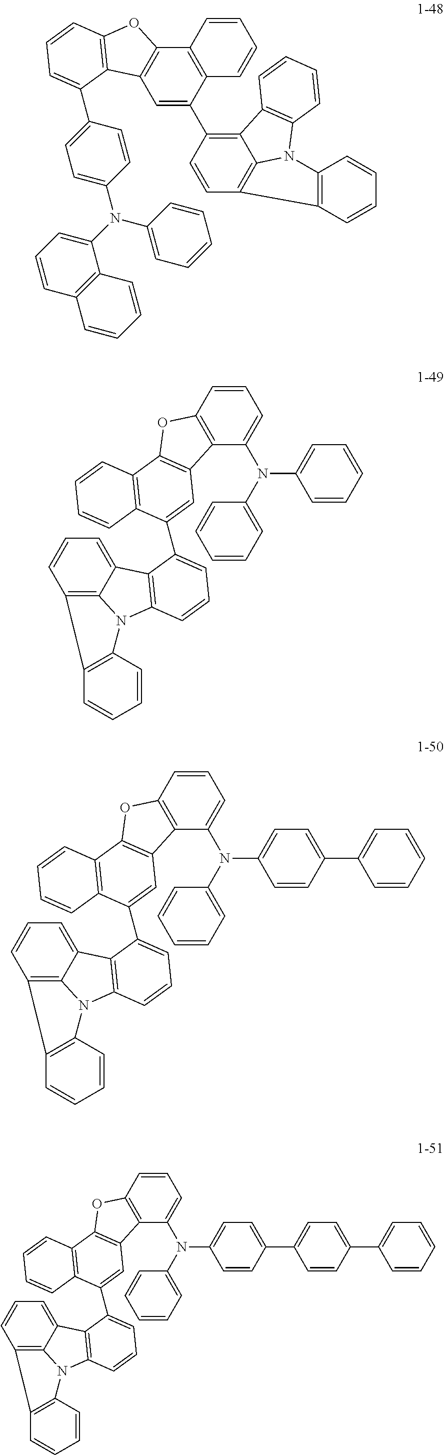 [SMILES: c1ccc(N(c2ccc(-c3cccc4oc5c6ccccc6c(-c6ccc7c8ccccc8n8c9ccccc9c6c78)cc5c34)cc2)c2cccc3ccccc23)cc1 ; c1ccc(N(c2ccccc2)c2cccc3oc4c5ccccc5c(-c5cccc6c5c5cccc7c8ccccc8n6c75)cc4c23)cc1 ; c1ccc(-c2ccc(N(c3ccccc3)c3cccc4oc5c6ccccc6c(-c6cccc7c6c6cccc8c9ccccc9n7c86)cc5c34)cc2)cc1 ; c1ccc(-c2ccc(-c3ccc(N(c4ccccc4)c4cccc5oc6c7ccccc7c(-c7cccc8c7c7cccc9c%10ccccc%10n8c97)cc6c45)cc3)cc2)cc1]

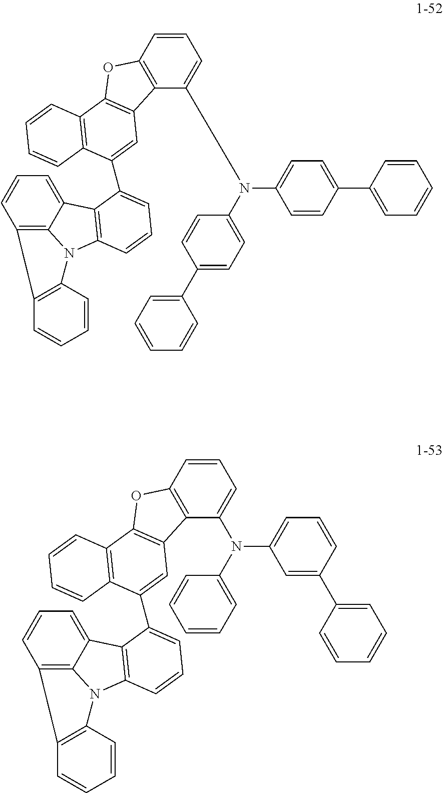 [SMILES: c1ccc(-c2ccc(N(c3ccc(-c4ccccc4)cc3)c3cccc4oc5c6ccccc6c(-c6cccc7c6c6cccc8c9ccccc9n7c86)cc5c34)cc2)cc1 ; c1ccc(-c2cccc(N(c3ccccc3)c3cccc4oc5c6ccccc6c(-c6cccc7c6c6cccc8c9ccccc9n7c86)cc5c34)c2)cc1]

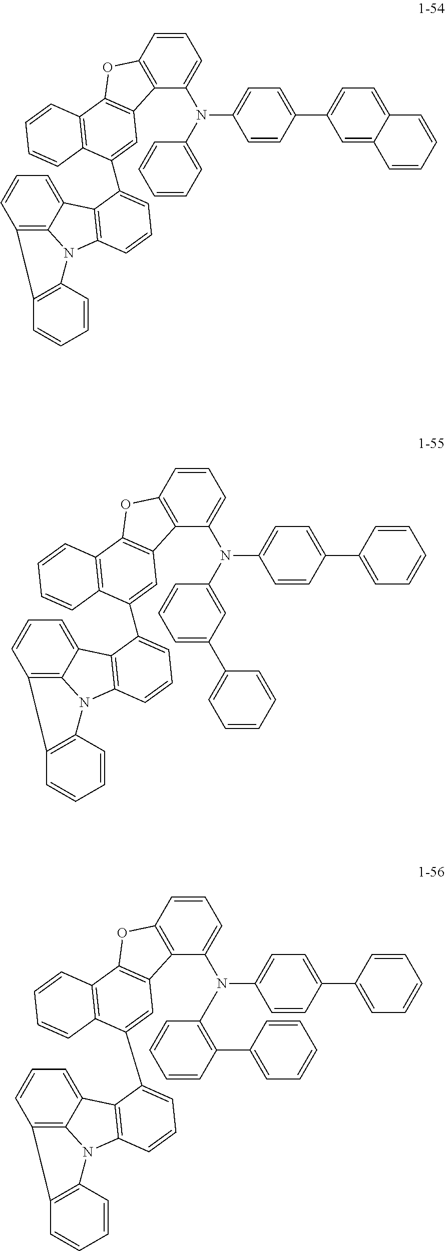 [SMILES: c1ccc(N(c2ccc(-c3ccc4ccccc4c3)cc2)c2cccc3oc4c5ccccc5c(-c5cccc6c5c5cccc7c8ccccc8n6c75)cc4c23)cc1 ; c1ccc(-c2ccc(N(c3cccc(-c4ccccc4)c3)c3cccc4oc5c6ccccc6c(-c6cccc7c6c6cccc8c9ccccc9n7c86)cc5c34)cc2)cc1 ; c1ccc(-c2ccc(N(c3ccccc3-c3ccccc3)c3cccc4oc5c6ccccc6c(-c6cccc7c6c6cccc8c9ccccc9n7c86)cc5c34)cc2)cc1]

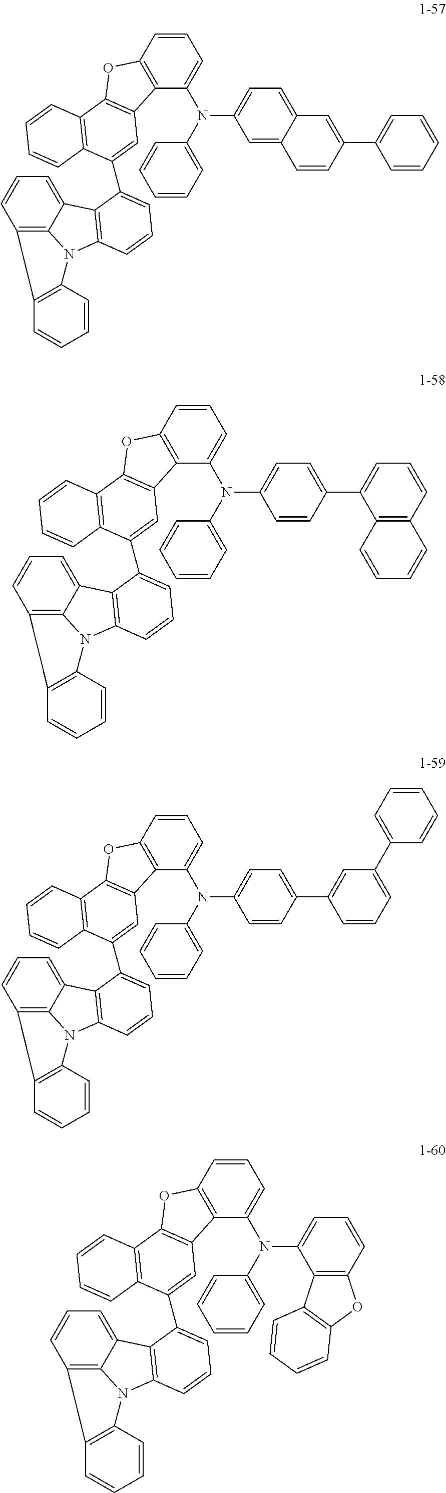 [SMILES: c1ccc(-c2ccc3cc(N(c4ccccc4)c4cccc5oc6c7ccccc7c(-c7cccc8c7c7cccc9c%10ccccc%10n8c97)cc6c45)ccc3c2)cc1 ; c1ccc(N(c2ccc(-c3cccc4ccccc34)cc2)c2cccc3oc4c5ccccc5c(-c5cccc6c5c5cccc7c8ccccc8n6c75)cc4c23)cc1 ; c1ccc(-c2cccc(-c3ccc(N(c4ccccc4)c4cccc5oc6c7ccccc7c(-c7cccc8c7c7cccc9c%10ccccc%10n8c97)cc6c45)cc3)c2)cc1 ; c1ccc(N(c2cccc3oc4ccccc4c23)c2cccc3oc4c5ccccc5c(-c5cccc6c5c5cccc7c8ccccc8n6c75)cc4c23)cc1]

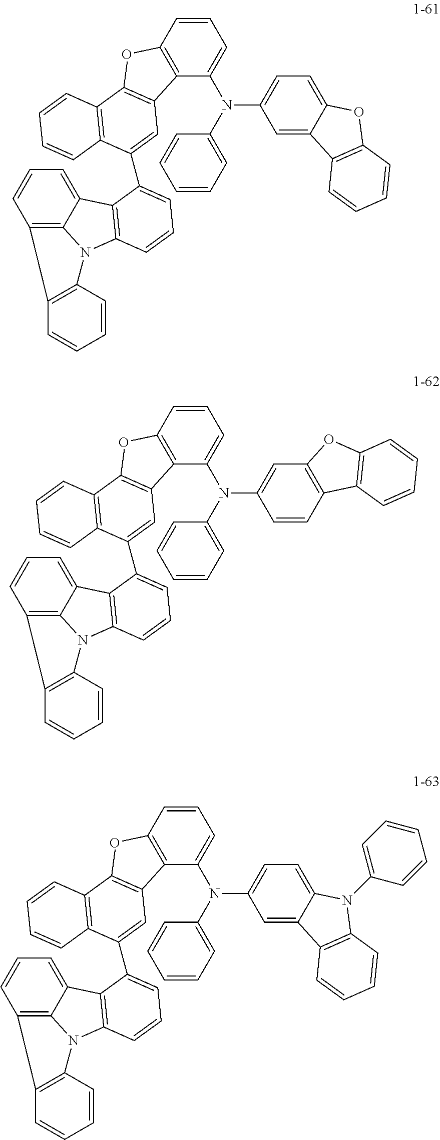 [SMILES: c1ccc(N(c2ccc3oc4ccccc4c3c2)c2cccc3oc4c5ccccc5c(-c5cccc6c5c5cccc7c8ccccc8n6c75)cc4c23)cc1 ; c1ccc(N(c2ccc3c(c2)oc2ccccc23)c2cccc3oc4c5ccccc5c(-c5cccc6c5c5cccc7c8ccccc8n6c75)cc4c23)cc1 ; c1ccc(N(c2ccc3c(c2)c2ccccc2n3-c2ccccc2)c2cccc3oc4c5ccccc5c(-c5cccc6c5c5cccc7c8ccccc8n6c75)cc4c23)cc1]

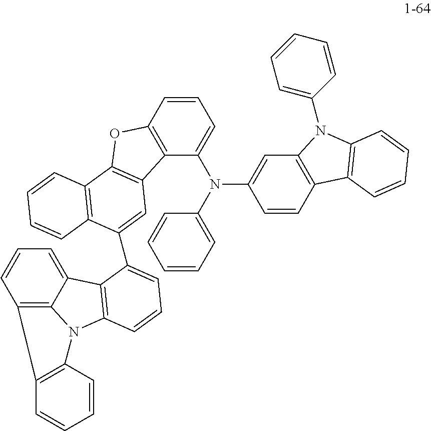 [SMILES: c1ccc(N(c2ccc3c4ccccc4n(-c4ccccc4)c3c2)c2cccc3oc4c5ccccc5c(-c5cccc6c5c5cccc7c8ccccc8n6c75)cc4c23)cc1]

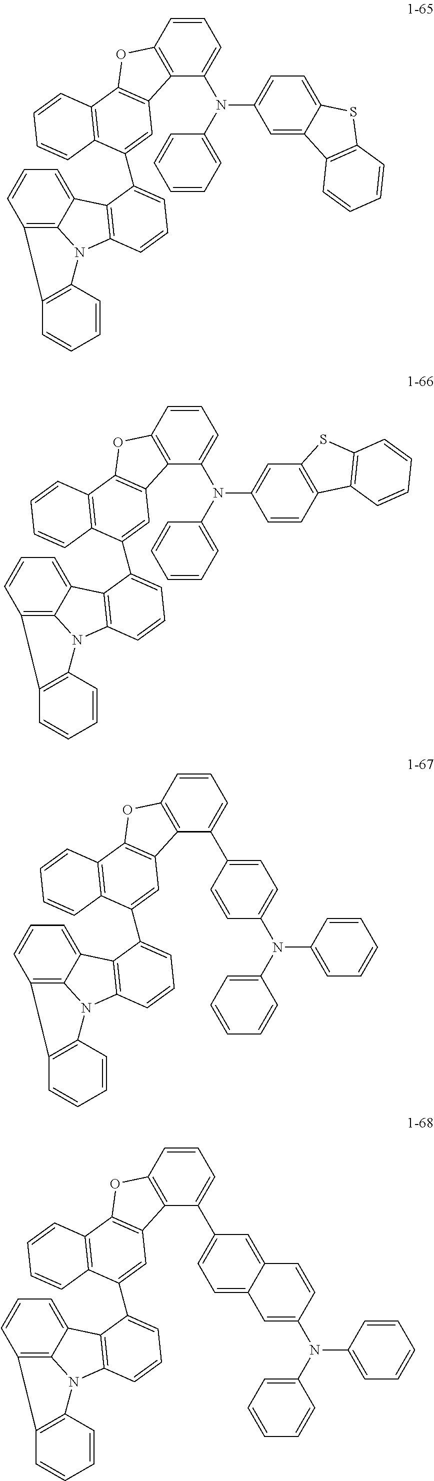 [SMILES: c1ccc(N(c2ccc3sc4ccccc4c3c2)c2cccc3oc4c5ccccc5c(-c5cccc6c5c5cccc7c8ccccc8n6c75)cc4c23)cc1 ; c1ccc(N(c2ccc3c(c2)sc2ccccc23)c2cccc3oc4c5ccccc5c(-c5cccc6c5c5cccc7c8ccccc8n6c75)cc4c23)cc1 ; c1ccc(N(c2ccccc2)c2ccc(-c3cccc4oc5c6ccccc6c(-c6cccc7c6c6cccc8c9ccccc9n7c86)cc5c34)cc2)cc1 ; c1ccc(N(c2ccccc2)c2ccc3cc(-c4cccc5oc6c7ccccc7c(-c7cccc8c7c7cccc9c%10ccccc%10n8c97)cc6c45)ccc3c2)cc1]

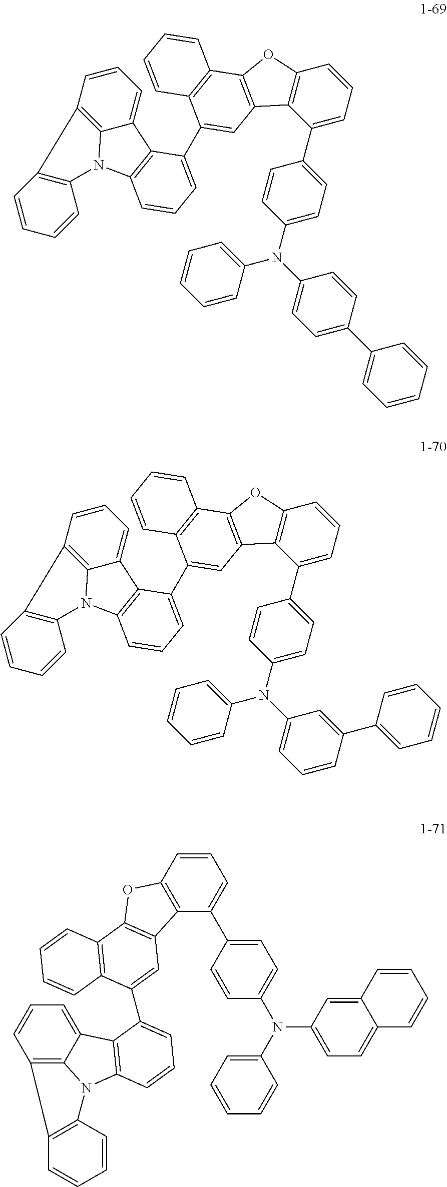 [SMILES: c1ccc(-c2ccc(N(c3ccccc3)c3ccc(-c4cccc5oc6c7ccccc7c(-c7cccc8c7c7cccc9c%10ccccc%10n8c97)cc6c45)cc3)cc2)cc1 ; c1ccc(-c2cccc(N(c3ccccc3)c3ccc(-c4cccc5oc6c7ccccc7c(-c7cccc8c7c7cccc9c%10ccccc%10n8c97)cc6c45)cc3)c2)cc1 ; c1ccc(N(c2ccc(-c3cccc4oc5c6ccccc6c(-c6cccc7c6c6cccc8c9ccccc9n7c86)cc5c34)cc2)c2ccc3ccccc3c2)cc1]

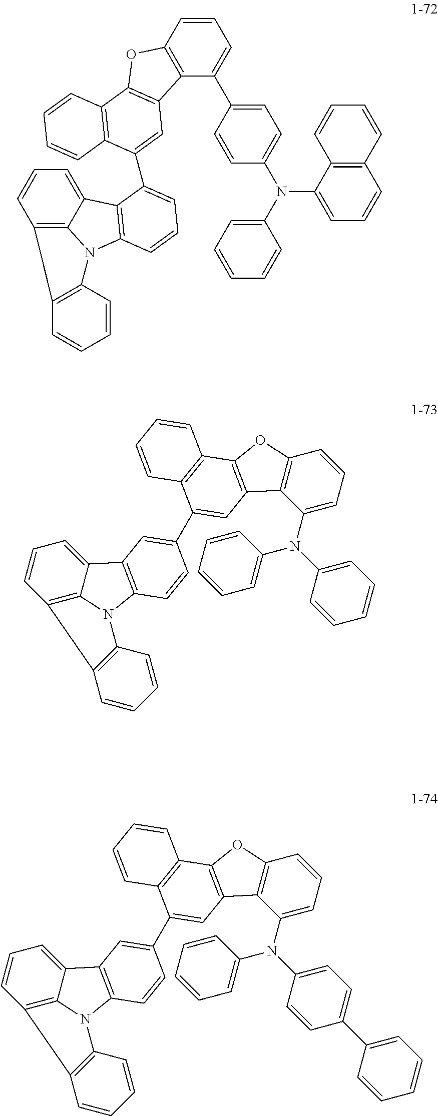 [SMILES: c1ccc(N(c2ccc(-c3cccc4oc5c6ccccc6c(-c6cccc7c6c6cccc8c9ccccc9n7c86)cc5c34)cc2)c2cccc3ccccc23)cc1 ; c1ccc(N(c2ccccc2)c2cccc3oc4c5ccccc5c(-c5ccc6c(c5)c5cccc7c8ccccc8n6c75)cc4c23)cc1 ; c1ccc(-c2ccc(N(c3ccccc3)c3cccc4oc5c6ccccc6c(-c6ccc7c(c6)c6cccc8c9ccccc9n7c86)cc5c34)cc2)cc1]

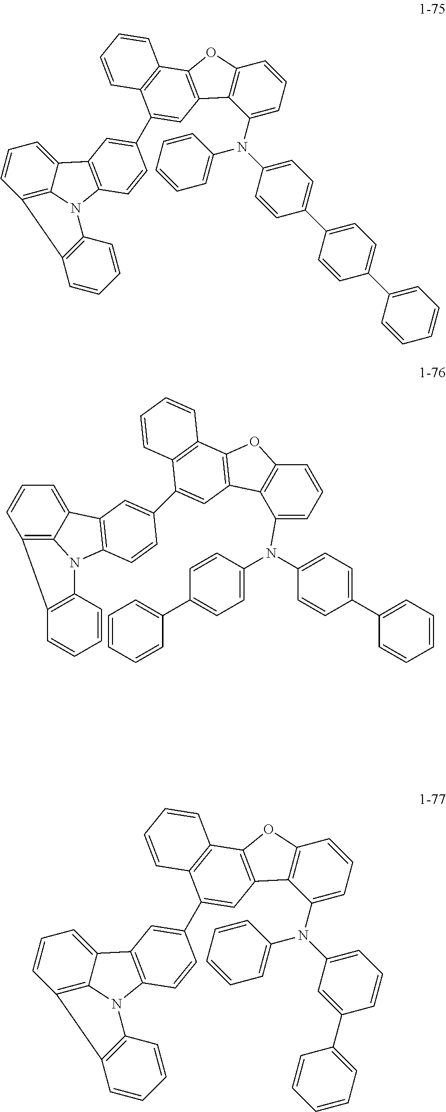 [SMILES: c1ccc(-c2ccc(-c3ccc(N(c4ccccc4)c4cccc5oc6c7ccccc7c(-c7ccc8c(c7)c7cccc9c%10ccccc%10n8c97)cc6c45)cc3)cc2)cc1 ; c1ccc(-c2ccc(N(c3ccc(-c4ccccc4)cc3)c3cccc4oc5c6ccccc6c(-c6ccc7c(c6)c6cccc8c9ccccc9n7c86)cc5c34)cc2)cc1 ; c1ccc(-c2cccc(N(c3ccccc3)c3cccc4oc5c6ccccc6c(-c6ccc7c(c6)c6cccc8c9ccccc9n7c86)cc5c34)c2)cc1]

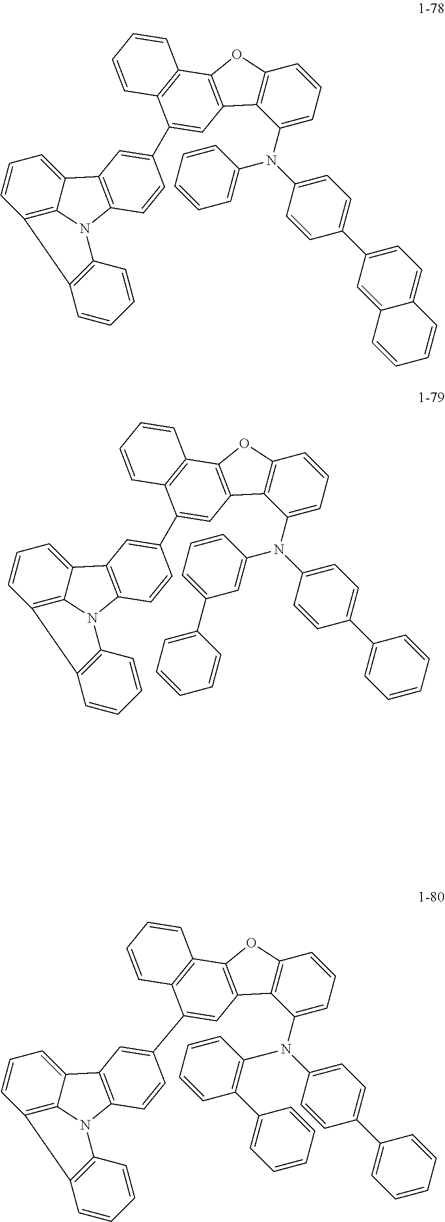 [SMILES: c1ccc(N(c2ccc(-c3ccc4ccccc4c3)cc2)c2cccc3oc4c5ccccc5c(-c5ccc6c(c5)c5cccc7c8ccccc8n6c75)cc4c23)cc1 ; c1ccc(-c2ccc(N(c3cccc(-c4ccccc4)c3)c3cccc4oc5c6ccccc6c(-c6ccc7c(c6)c6cccc8c9ccccc9n7c86)cc5c34)cc2)cc1 ; c1ccc(-c2ccc(N(c3ccccc3-c3ccccc3)c3cccc4oc5c6ccccc6c(-c6ccc7c(c6)c6cccc8c9ccccc9n7c86)cc5c34)cc2)cc1]

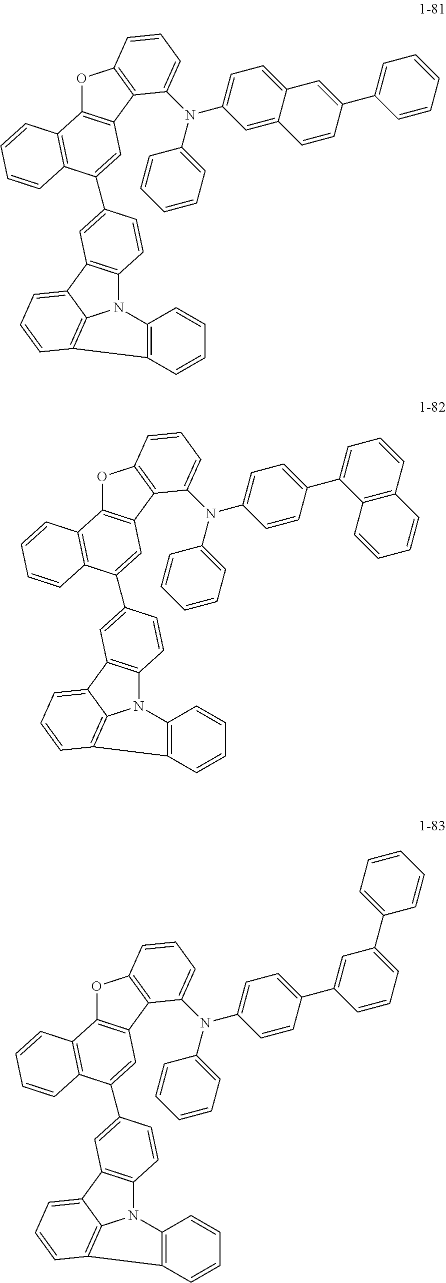 [SMILES: c1ccc(-c2ccc3cc(N(c4ccccc4)c4cccc5oc6c7ccccc7c(-c7ccc8c(c7)c7cccc9c%10ccccc%10n8c97)cc6c45)ccc3c2)cc1 ; c1ccc(N(c2ccc(-c3cccc4ccccc34)cc2)c2cccc3oc4c5ccccc5c(-c5ccc6c(c5)c5cccc7c8ccccc8n6c75)cc4c23)cc1 ; c1ccc(-c2cccc(-c3ccc(N(c4ccccc4)c4cccc5oc6c7ccccc7c(-c7ccc8c(c7)c7cccc9c%10ccccc%10n8c97)cc6c45)cc3)c2)cc1]

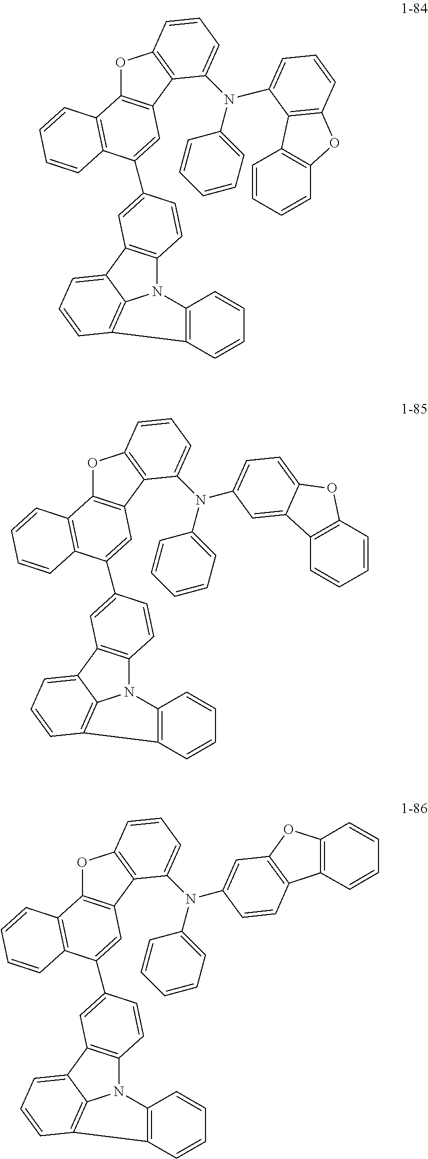 [SMILES: c1ccc(N(c2cccc3oc4ccccc4c23)c2cccc3oc4c5ccccc5c(-c5ccc6c(c5)c5cccc7c8ccccc8n6c75)cc4c23)cc1 ; c1ccc(N(c2ccc3oc4ccccc4c3c2)c2cccc3oc4c5ccccc5c(-c5ccc6c(c5)c5cccc7c8ccccc8n6c75)cc4c23)cc1 ; c1ccc(N(c2ccc3c(c2)oc2ccccc23)c2cccc3oc4c5ccccc5c(-c5ccc6c(c5)c5cccc7c8ccccc8n6c75)cc4c23)cc1]

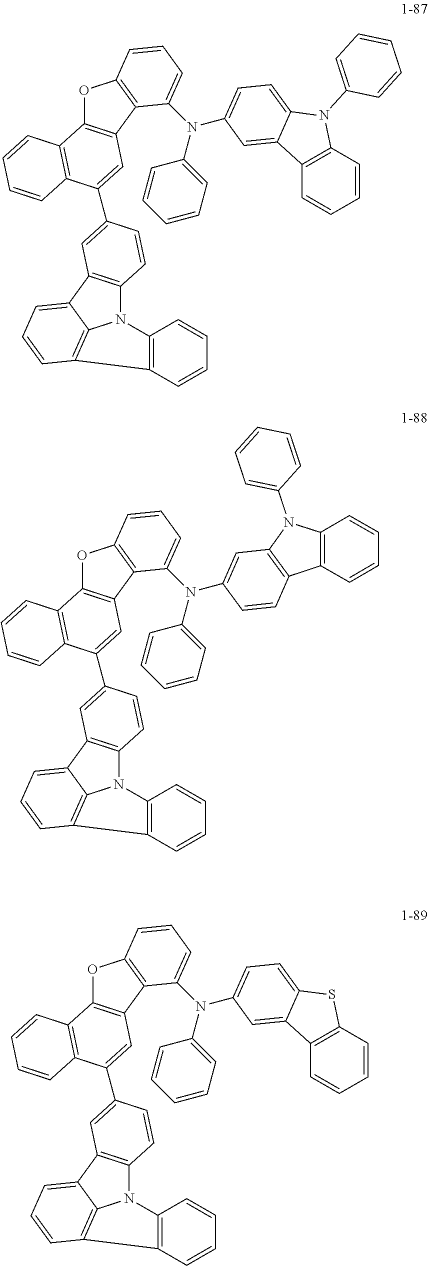 [SMILES: c1ccc(N(c2ccc3c(c2)c2ccccc2n3-c2ccccc2)c2cccc3oc4c5ccccc5c(-c5ccc6c(c5)c5cccc7c8ccccc8n6c75)cc4c23)cc1 ; c1ccc(N(c2ccc3c4ccccc4n(-c4ccccc4)c3c2)c2cccc3oc4c5ccccc5c(-c5ccc6c(c5)c5cccc7c8ccccc8n6c75)cc4c23)cc1 ; c1ccc(N(c2ccc3sc4ccccc4c3c2)c2cccc3oc4c5ccccc5c(-c5ccc6c(c5)c5cccc7c8ccccc8n6c75)cc4c23)cc1]

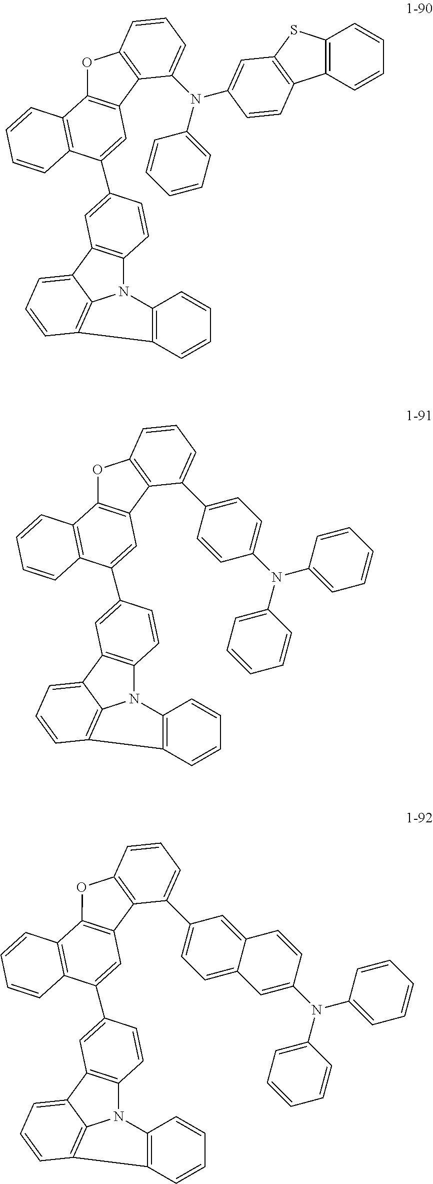 [SMILES: c1ccc(N(c2ccc3c(c2)sc2ccccc23)c2cccc3oc4c5ccccc5c(-c5ccc6c(c5)c5cccc7c8ccccc8n6c75)cc4c23)cc1 ; c1ccc(N(c2ccccc2)c2ccc(-c3cccc4oc5c6ccccc6c(-c6ccc7c(c6)c6cccc8c9ccccc9n7c86)cc5c34)cc2)cc1 ; c1ccc(N(c2ccccc2)c2ccc3cc(-c4cccc5oc6c7ccccc7c(-c7ccc8c(c7)c7cccc9c%10ccccc%10n8c97)cc6c45)ccc3c2)cc1]

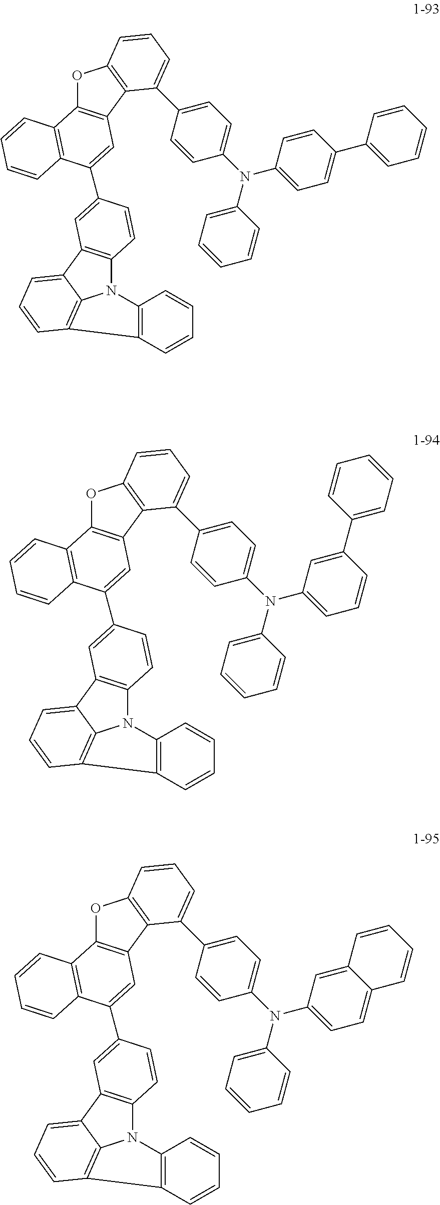 [SMILES: c1ccc(-c2ccc(N(c3ccccc3)c3ccc(-c4cccc5oc6c7ccccc7c(-c7ccc8c(c7)c7cccc9c%10ccccc%10n8c97)cc6c45)cc3)cc2)cc1 ; c1ccc(-c2cccc(N(c3ccccc3)c3ccc(-c4cccc5oc6c7ccccc7c(-c7ccc8c(c7)c7cccc9c%10ccccc%10n8c97)cc6c45)cc3)c2)cc1 ; c1ccc(N(c2ccc(-c3cccc4oc5c6ccccc6c(-c6ccc7c(c6)c6cccc8c9ccccc9n7c86)cc5c34)cc2)c2ccc3ccccc3c2)cc1]

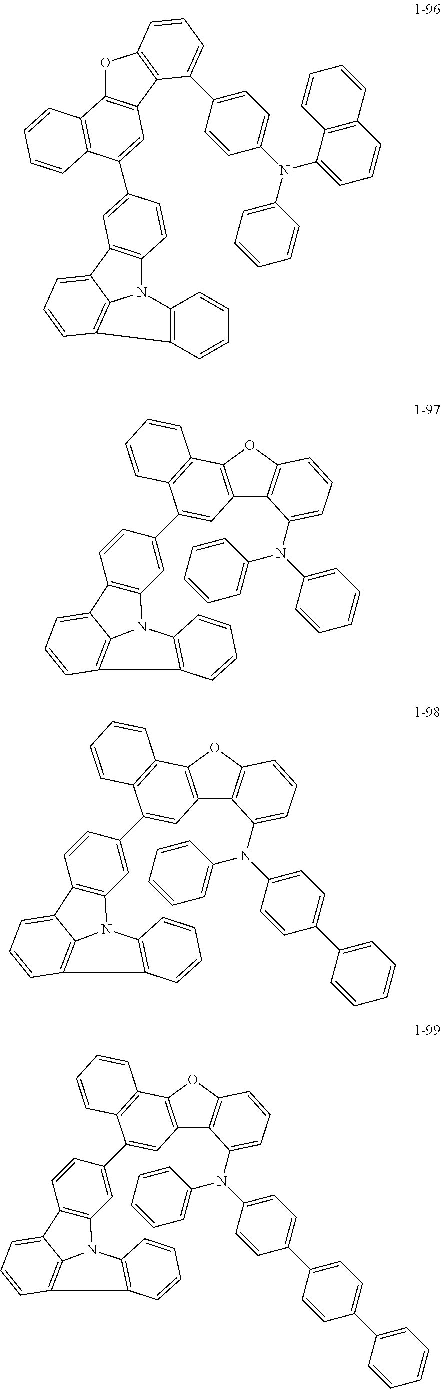 [SMILES: c1ccc(N(c2ccc(-c3cccc4oc5c6ccccc6c(-c6ccc7c(c6)c6cccc8c9ccccc9n7c86)cc5c34)cc2)c2cccc3ccccc23)cc1 ; c1ccc(N(c2ccccc2)c2cccc3oc4c5ccccc5c(-c5ccc6c7cccc8c9ccccc9n(c6c5)c87)cc4c23)cc1 ; c1ccc(-c2ccc(N(c3ccccc3)c3cccc4oc5c6ccccc6c(-c6ccc7c8cccc9c%10ccccc%10n(c7c6)c98)cc5c34)cc2)cc1 ; c1ccc(-c2ccc(-c3ccc(N(c4ccccc4)c4cccc5oc6c7ccccc7c(-c7ccc8c9cccc%10c%11ccccc%11n(c8c7)c%109)cc6c45)cc3)cc2)cc1]

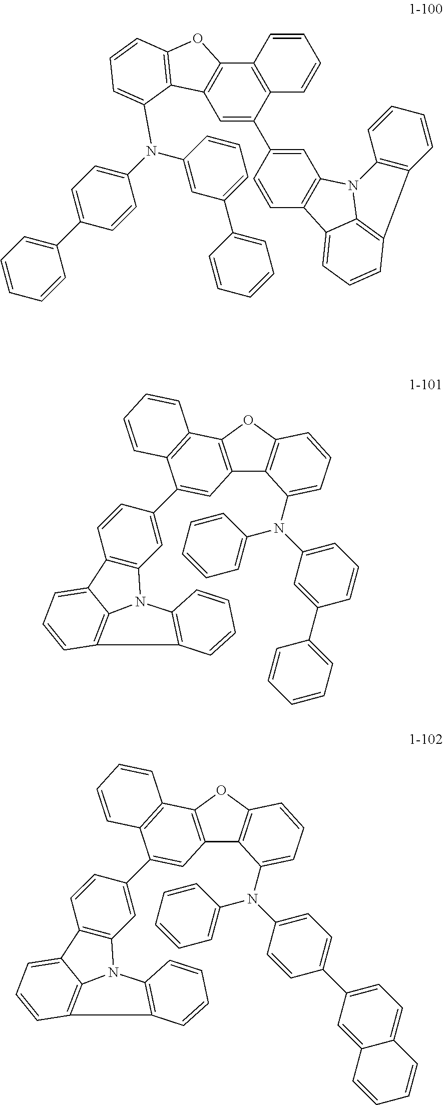 [SMILES: c1ccc(-c2ccc(N(c3cccc(-c4ccccc4)c3)c3cccc4oc5c6ccccc6c(-c6ccc7c8cccc9c%10ccccc%10n(c7c6)c98)cc5c34)cc2)cc1 ; c1ccc(-c2cccc(N(c3ccccc3)c3cccc4oc5c6ccccc6c(-c6ccc7c8cccc9c%10ccccc%10n(c7c6)c98)cc5c34)c2)cc1 ; c1ccc(N(c2ccc(-c3ccc4ccccc4c3)cc2)c2cccc3oc4c5ccccc5c(-c5ccc6c7cccc8c9ccccc9n(c6c5)c87)cc4c23)cc1]

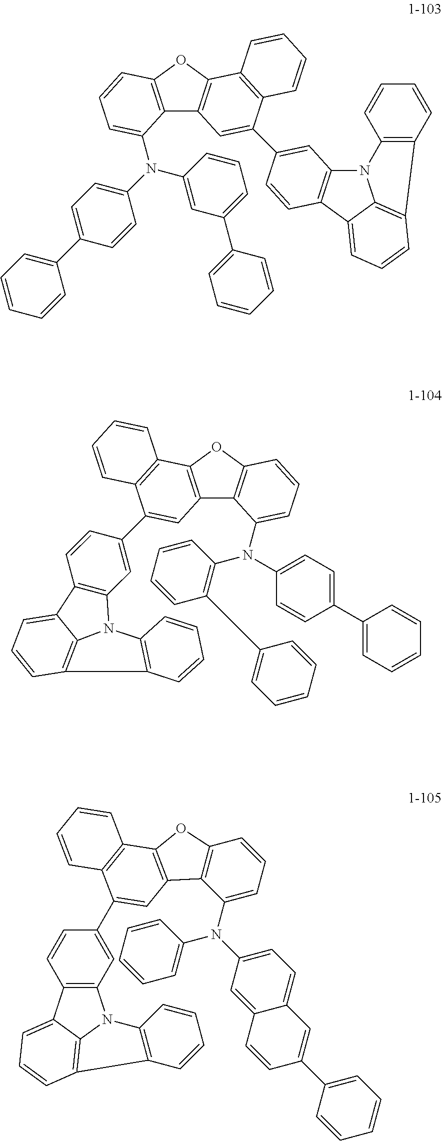 [SMILES: c1ccc(-c2ccc(N(c3cccc(-c4ccccc4)c3)c3cccc4oc5c6ccccc6c(-c6ccc7c8cccc9c%10ccccc%10n(c7c6)c98)cc5c34)cc2)cc1 ; c1ccc(-c2ccc(N(c3ccccc3-c3ccccc3)c3cccc4oc5c6ccccc6c(-c6ccc7c8cccc9c%10ccccc%10n(c7c6)c98)cc5c34)cc2)cc1 ; c1ccc(-c2ccc3cc(N(c4ccccc4)c4cccc5oc6c7ccccc7c(-c7ccc8c9cccc%10c%11ccccc%11n(c8c7)c%109)cc6c45)ccc3c2)cc1]

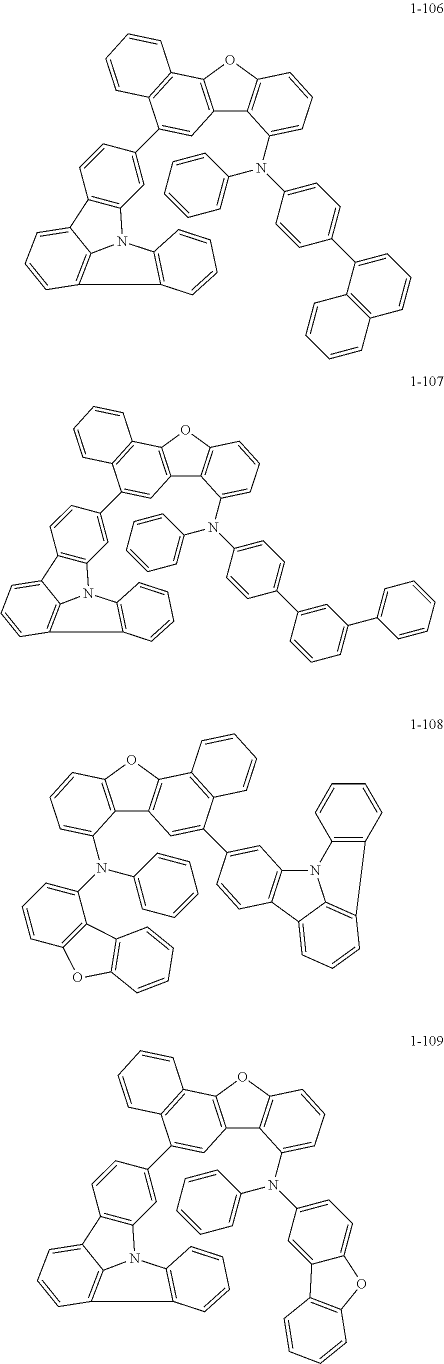 [SMILES: c1ccc(N(c2ccc(-c3cccc4ccccc34)cc2)c2cccc3oc4c5ccccc5c(-c5ccc6c7cccc8c9ccccc9n(c6c5)c87)cc4c23)cc1 ; c1ccc(-c2cccc(-c3ccc(N(c4ccccc4)c4cccc5oc6c7ccccc7c(-c7ccc8c9cccc%10c%11ccccc%11n(c8c7)c%109)cc6c45)cc3)c2)cc1 ; c1ccc(N(c2cccc3oc4ccccc4c23)c2cccc3oc4c5ccccc5c(-c5ccc6c7cccc8c9ccccc9n(c6c5)c87)cc4c23)cc1 ; c1ccc(N(c2ccc3oc4ccccc4c3c2)c2cccc3oc4c5ccccc5c(-c5ccc6c7cccc8c9ccccc9n(c6c5)c87)cc4c23)cc1]

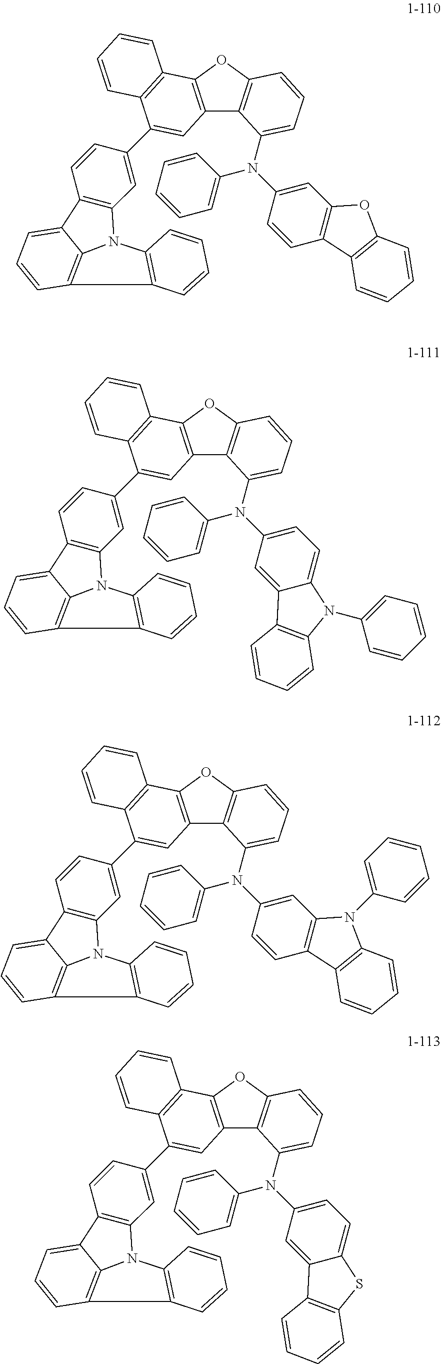 [SMILES: c1ccc(N(c2ccc3c(c2)oc2ccccc23)c2cccc3oc4c5ccccc5c(-c5ccc6c7cccc8c9ccccc9n(c6c5)c87)cc4c23)cc1 ; c1ccc(N(c2ccc3c(c2)c2ccccc2n3-c2ccccc2)c2cccc3oc4c5ccccc5c(-c5ccc6c7cccc8c9ccccc9n(c6c5)c87)cc4c23)cc1 ; c1ccc(N(c2ccc3c4ccccc4n(-c4ccccc4)c3c2)c2cccc3oc4c5ccccc5c(-c5ccc6c7cccc8c9ccccc9n(c6c5)c87)cc4c23)cc1 ; c1ccc(N(c2ccc3sc4ccccc4c3c2)c2cccc3oc4c5ccccc5c(-c5ccc6c7cccc8c9ccccc9n(c6c5)c87)cc4c23)cc1]

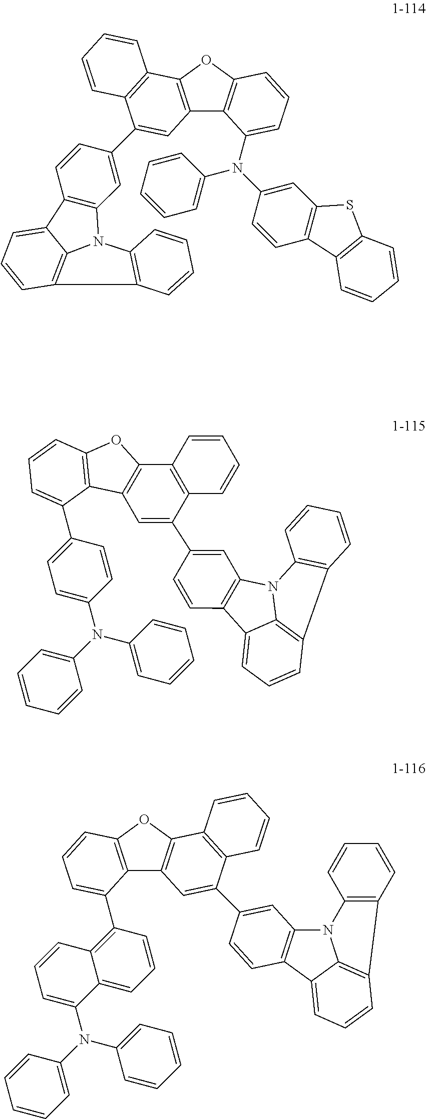 [SMILES: c1ccc(N(c2ccc3c(c2)sc2ccccc23)c2cccc3oc4c5ccccc5c(-c5ccc6c7cccc8c9ccccc9n(c6c5)c87)cc4c23)cc1 ; c1ccc(N(c2ccccc2)c2ccc(-c3cccc4oc5c6ccccc6c(-c6ccc7c8cccc9c%10ccccc%10n(c7c6)c98)cc5c34)cc2)cc1 ; c1ccc(N(c2ccccc2)c2cccc3c(-c4cccc5oc6c7ccccc7c(-c7ccc8c9cccc%10c%11ccccc%11n(c8c7)c%109)cc6c45)cccc23)cc1]

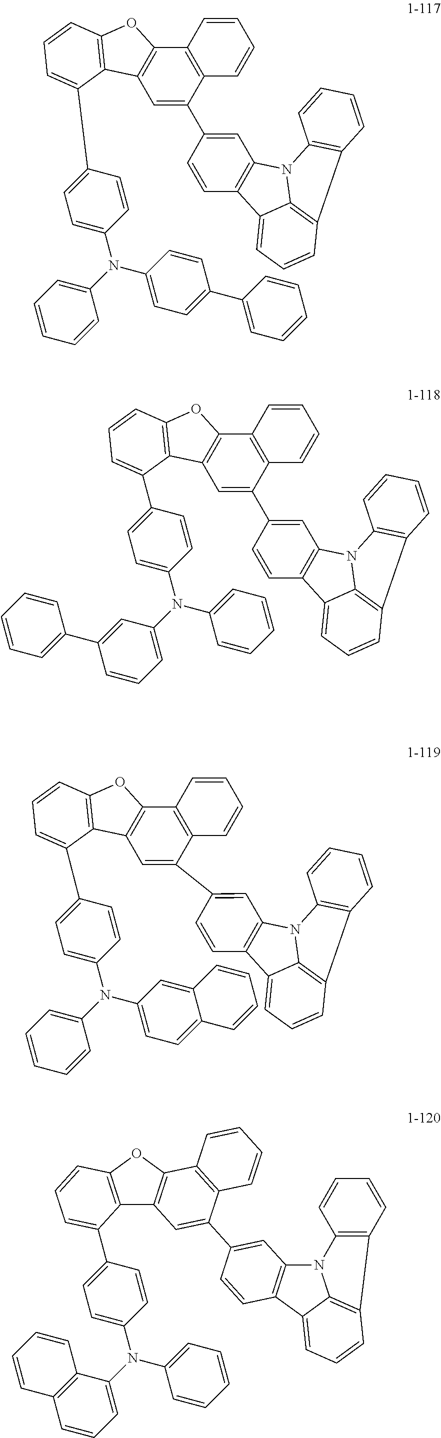 [SMILES: c1ccc(-c2ccc(N(c3ccccc3)c3ccc(-c4cccc5oc6c7ccccc7c(-c7ccc8c9cccc%10c%11ccccc%11n(c8c7)c%109)cc6c45)cc3)cc2)cc1 ; c1ccc(-c2cccc(N(c3ccccc3)c3ccc(-c4cccc5oc6c7ccccc7c(-c7ccc8c9cccc%10c%11ccccc%11n(c8c7)c%109)cc6c45)cc3)c2)cc1 ; c1ccc(N(c2ccc(-c3cccc4oc5c6ccccc6c(-c6ccc7c8cccc9c%10ccccc%10n(c7c6)c98)cc5c34)cc2)c2ccc3ccccc3c2)cc1 ; c1ccc(N(c2ccc(-c3cccc4oc5c6ccccc6c(-c6ccc7c8cccc9c%10ccccc%10n(c7c6)c98)cc5c34)cc2)c2cccc3ccccc23)cc1]

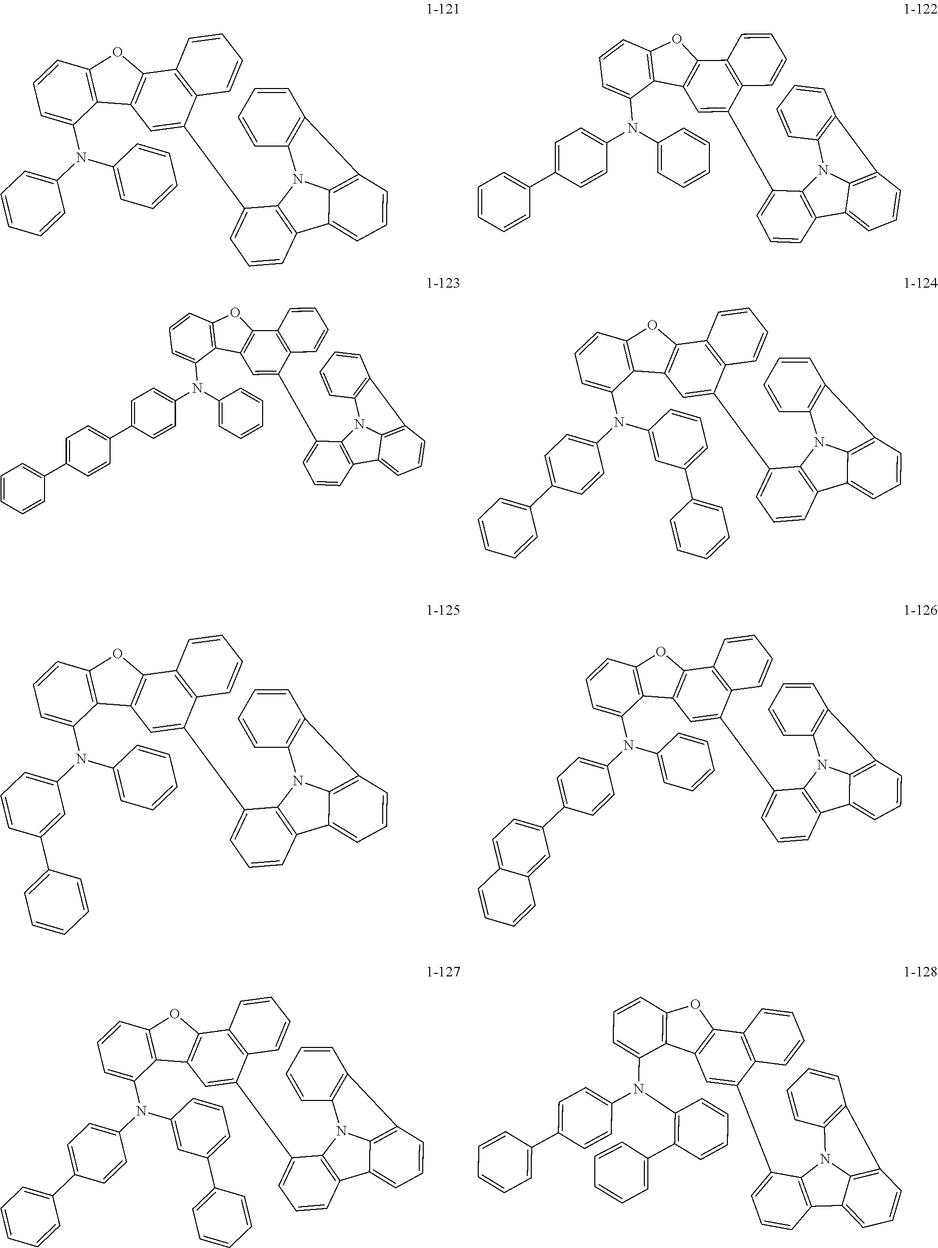

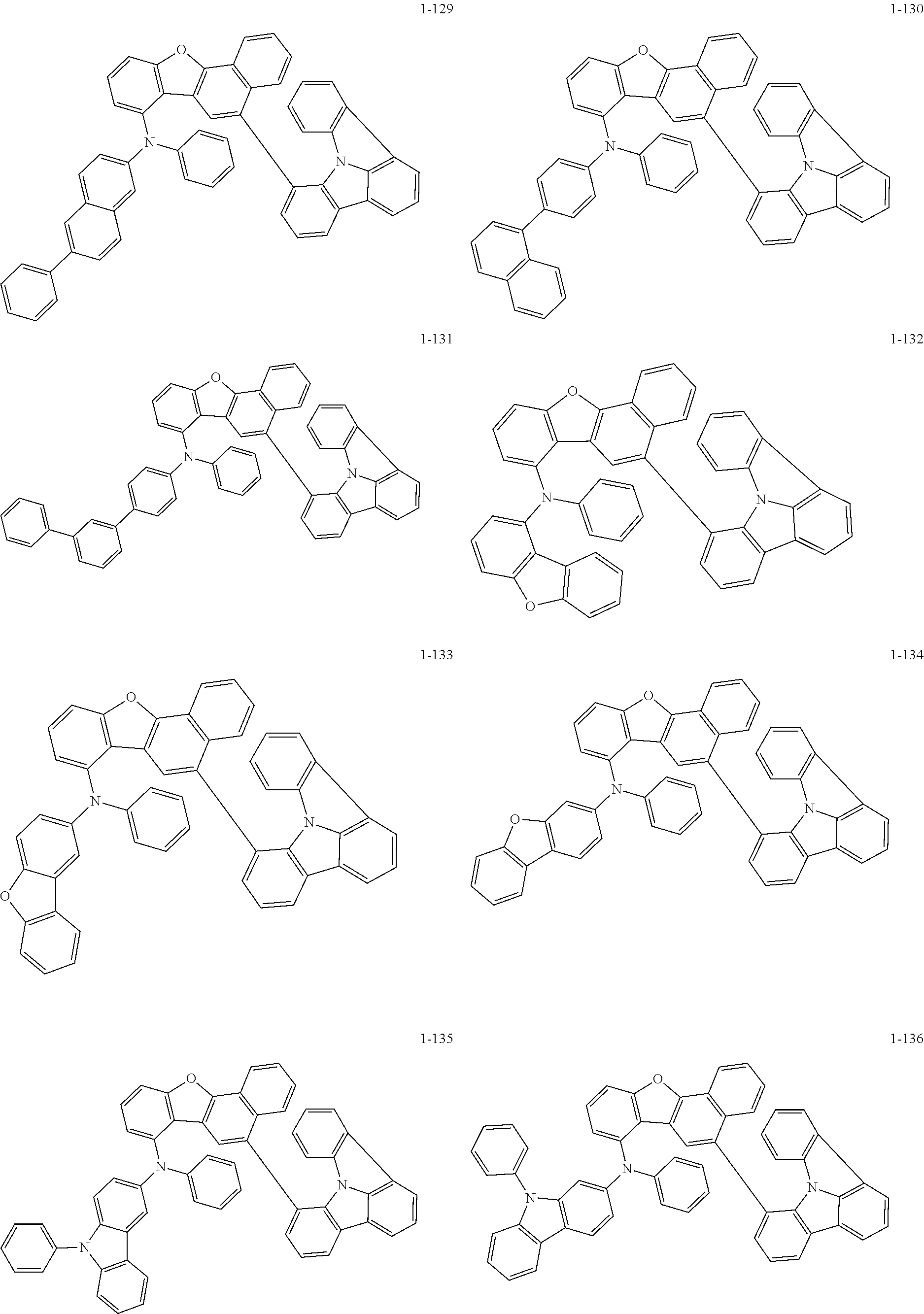

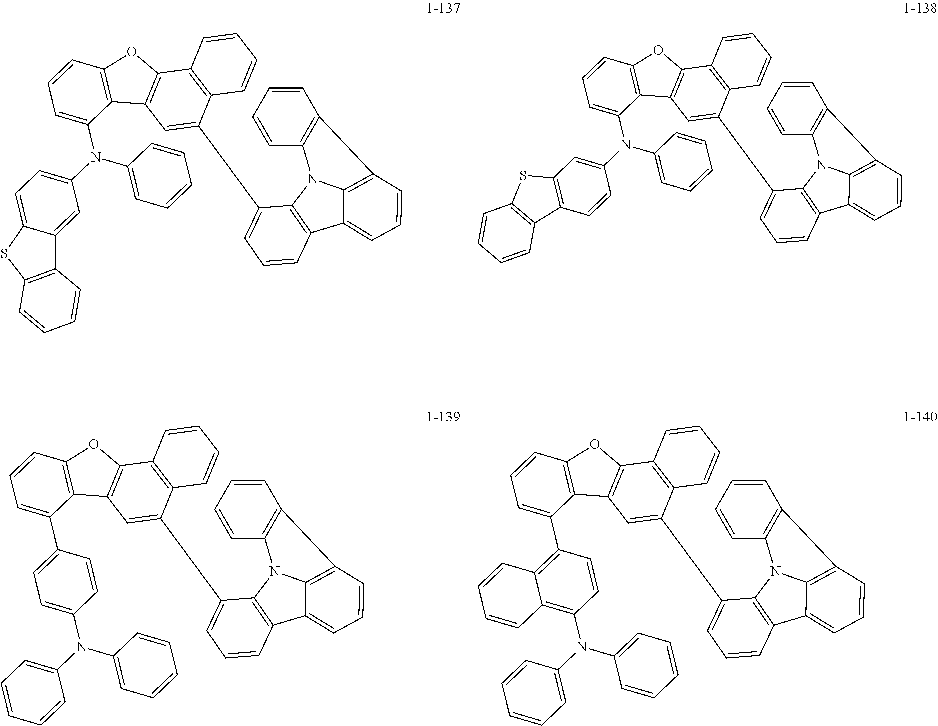 [SMILES: c1ccc(N(c2ccc3sc4ccccc4c3c2)c2cccc3oc4c5ccccc5c(-c5cccc6c7cccc8c9ccccc9n(c56)c87)cc4c23)cc1 ; c1ccc(N(c2ccc3c(c2)sc2ccccc23)c2cccc3oc4c5ccccc5c(-c5cccc6c7cccc8c9ccccc9n(c56)c87)cc4c23)cc1 ; c1ccc(N(c2ccccc2)c2ccc(-c3cccc4oc5c6ccccc6c(-c6cccc7c8cccc9c%10ccccc%10n(c67)c98)cc5c34)cc2)cc1 ; c1ccc(N(c2ccccc2)c2ccc(-c3cccc4oc5c6ccccc6c(-c6cccc7c8cccc9c%10ccccc%10n(c67)c98)cc5c34)c3ccccc23)cc1]

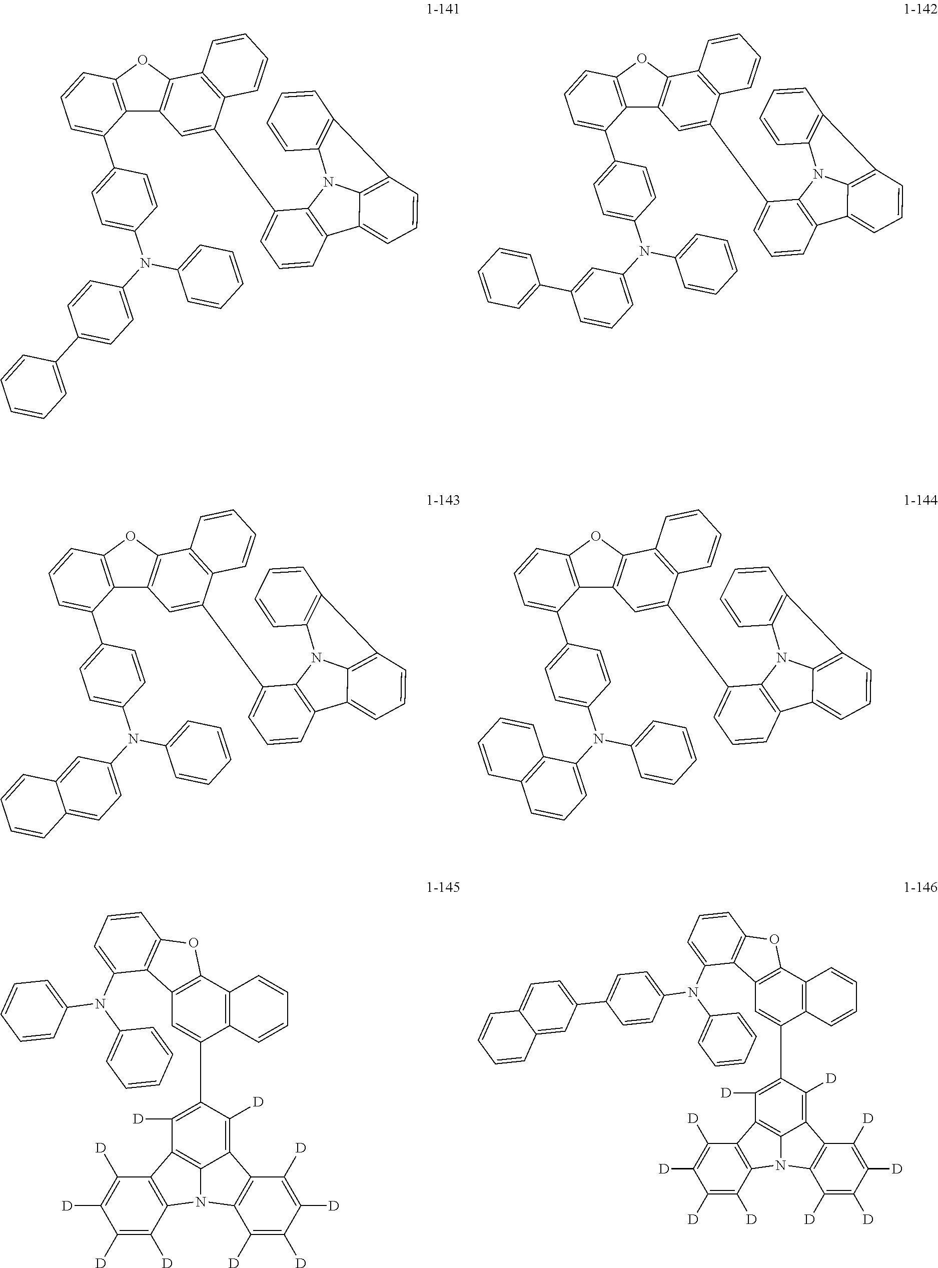

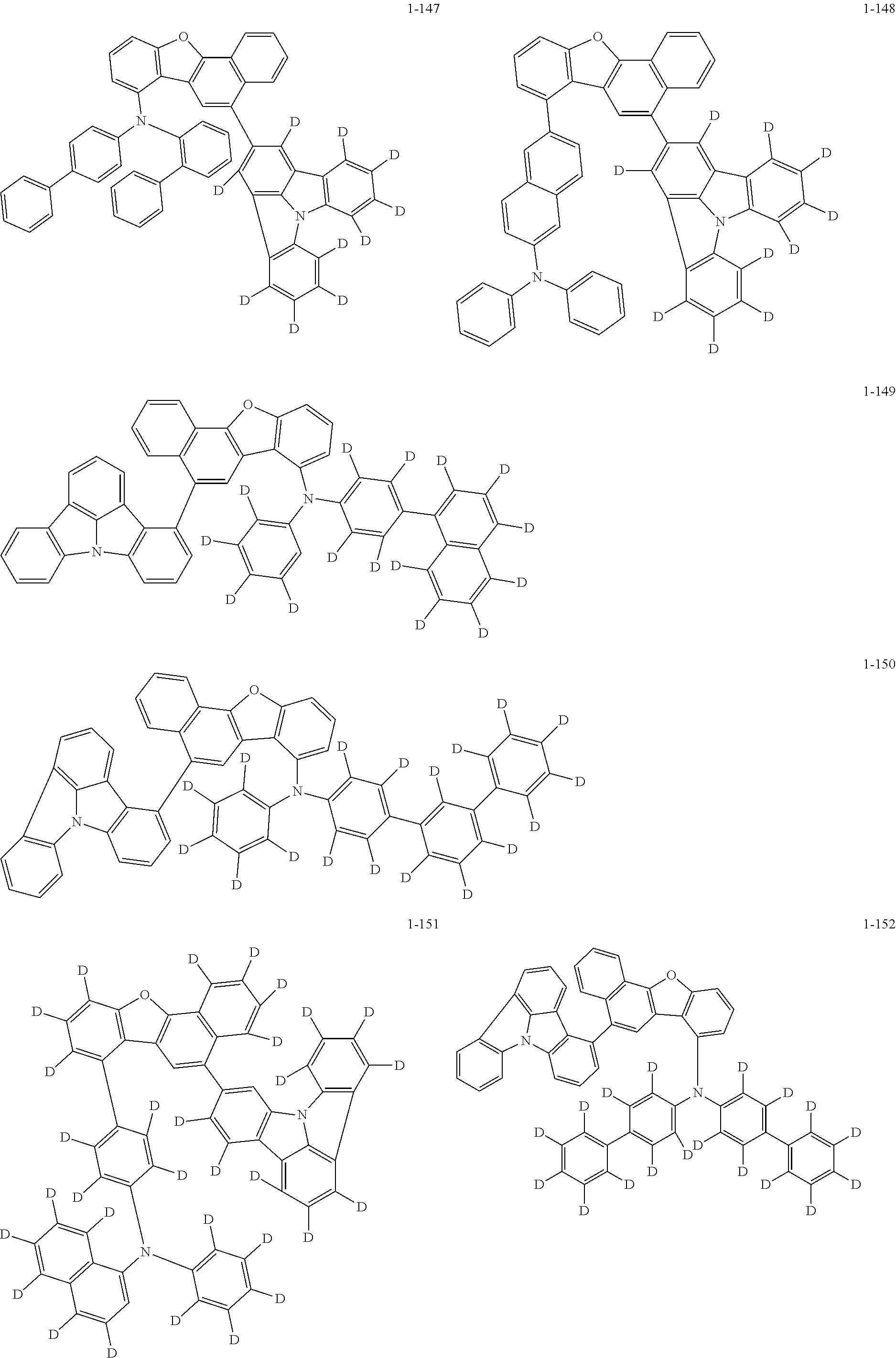

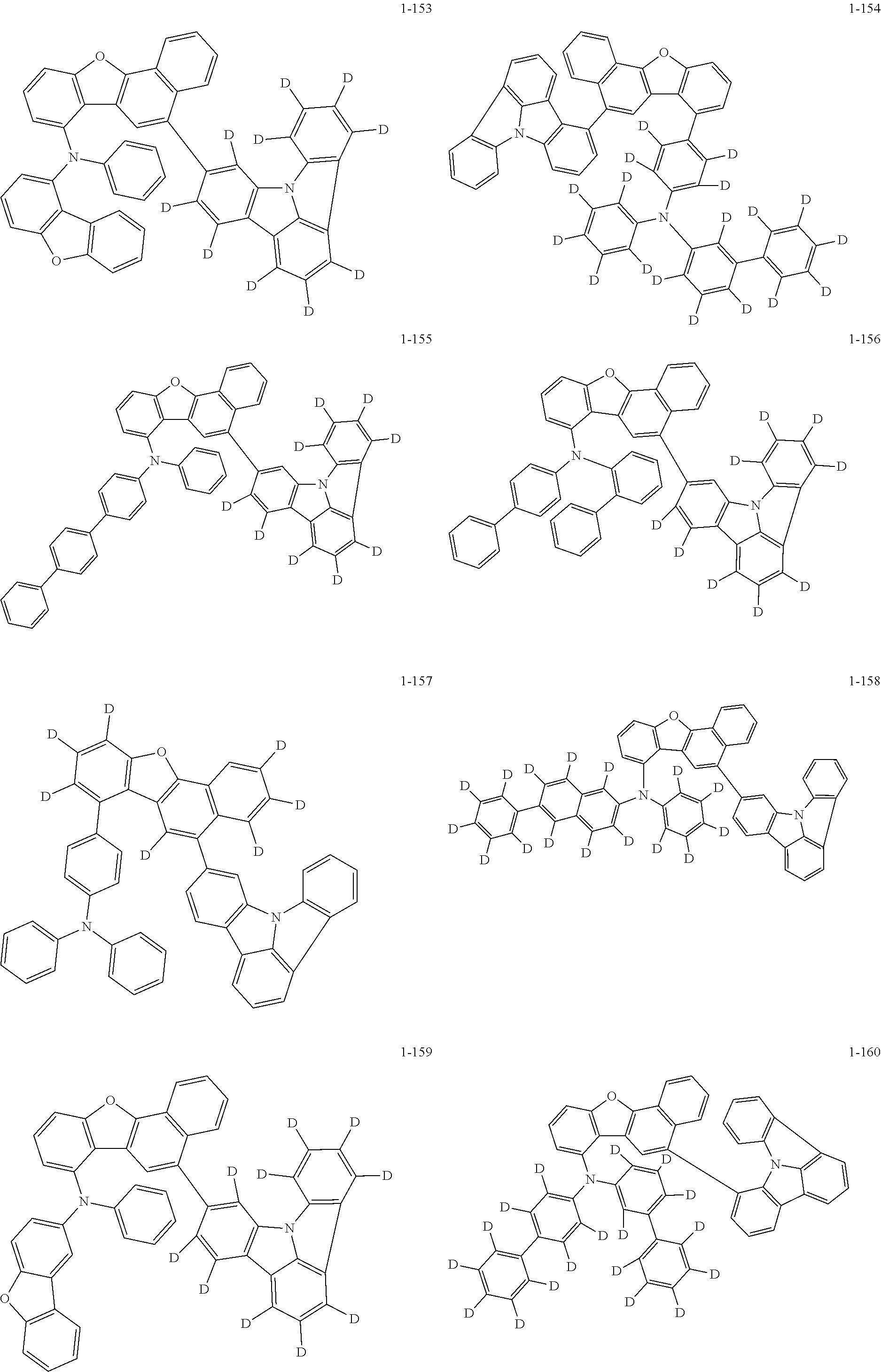

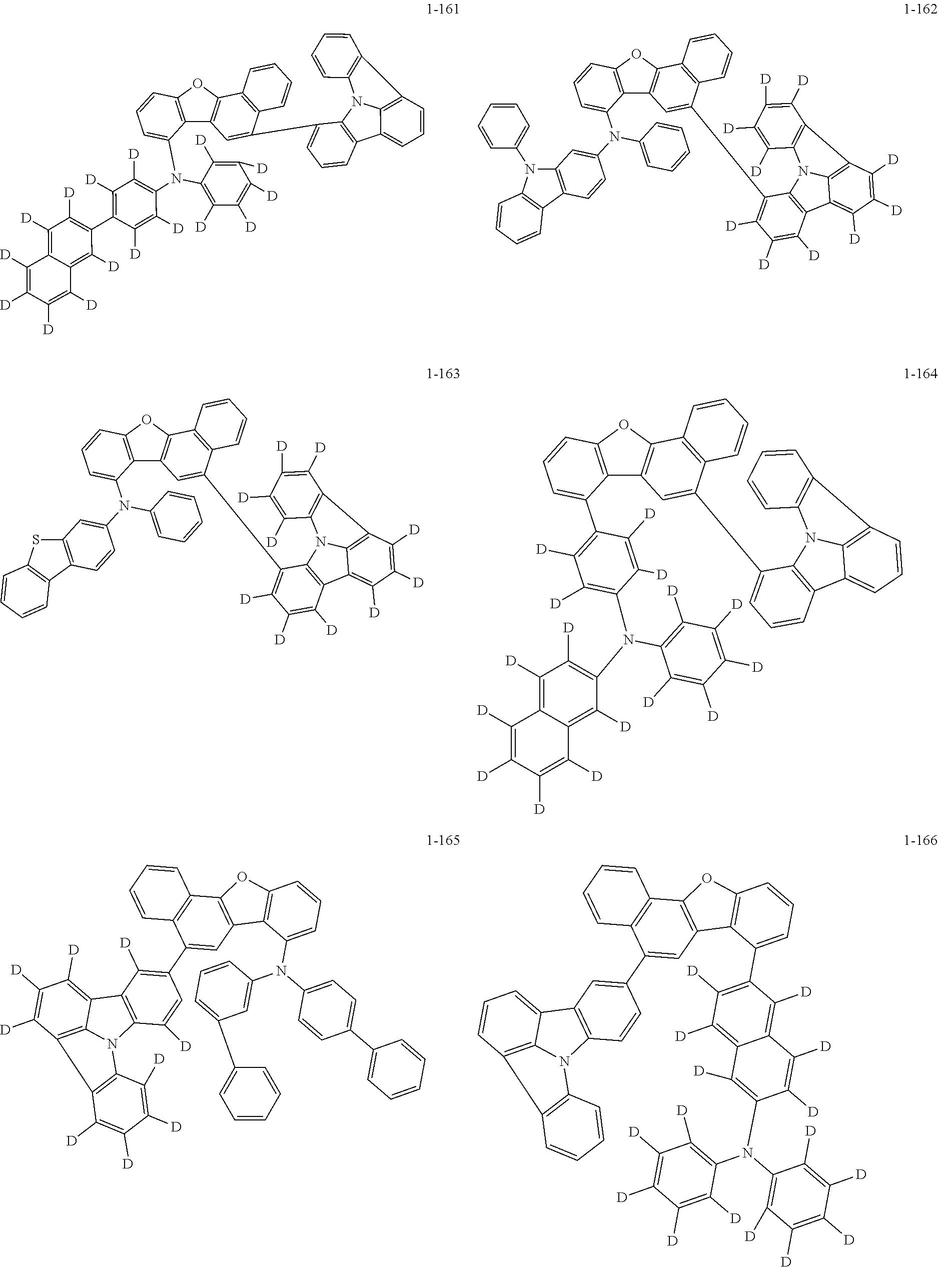

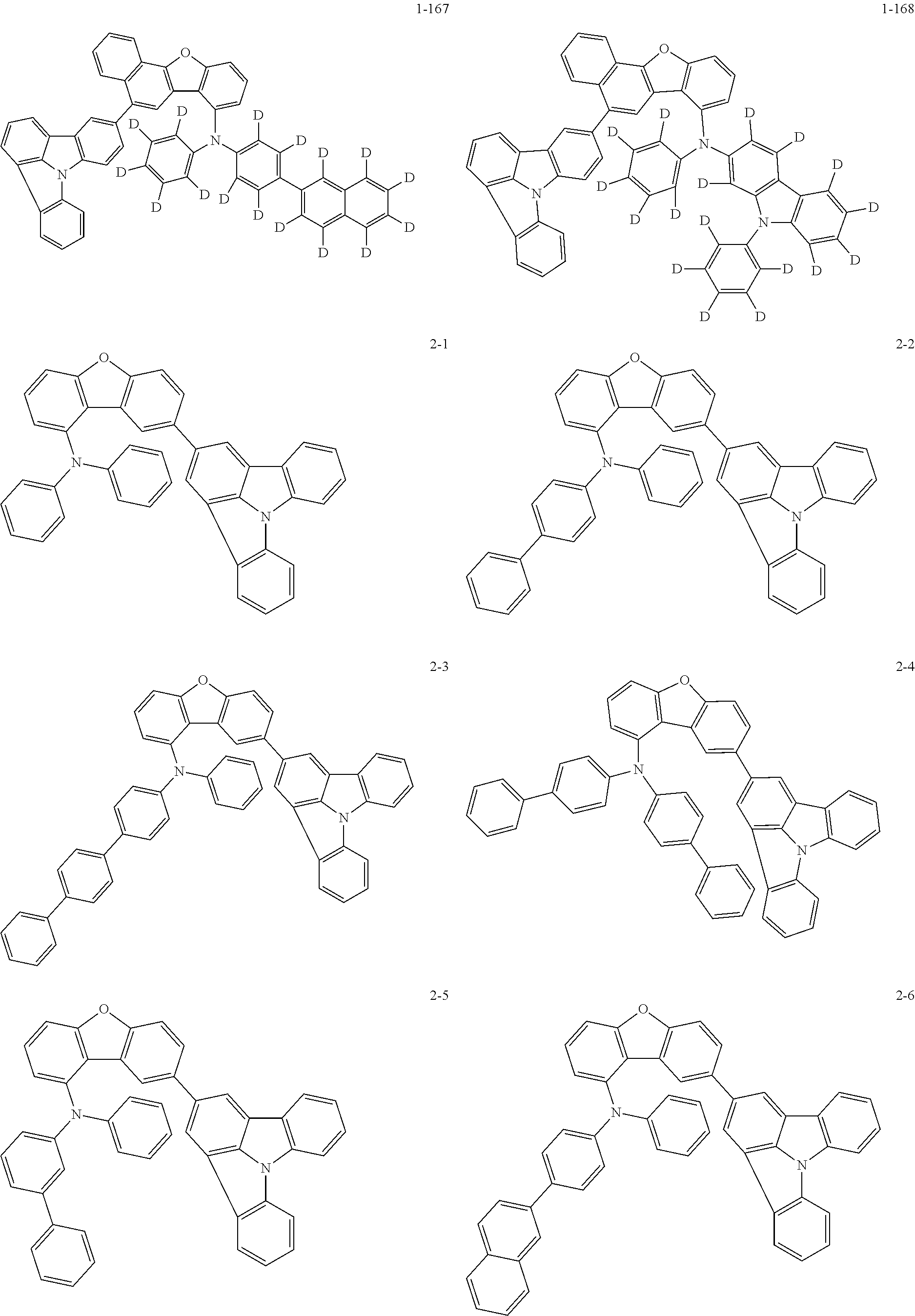

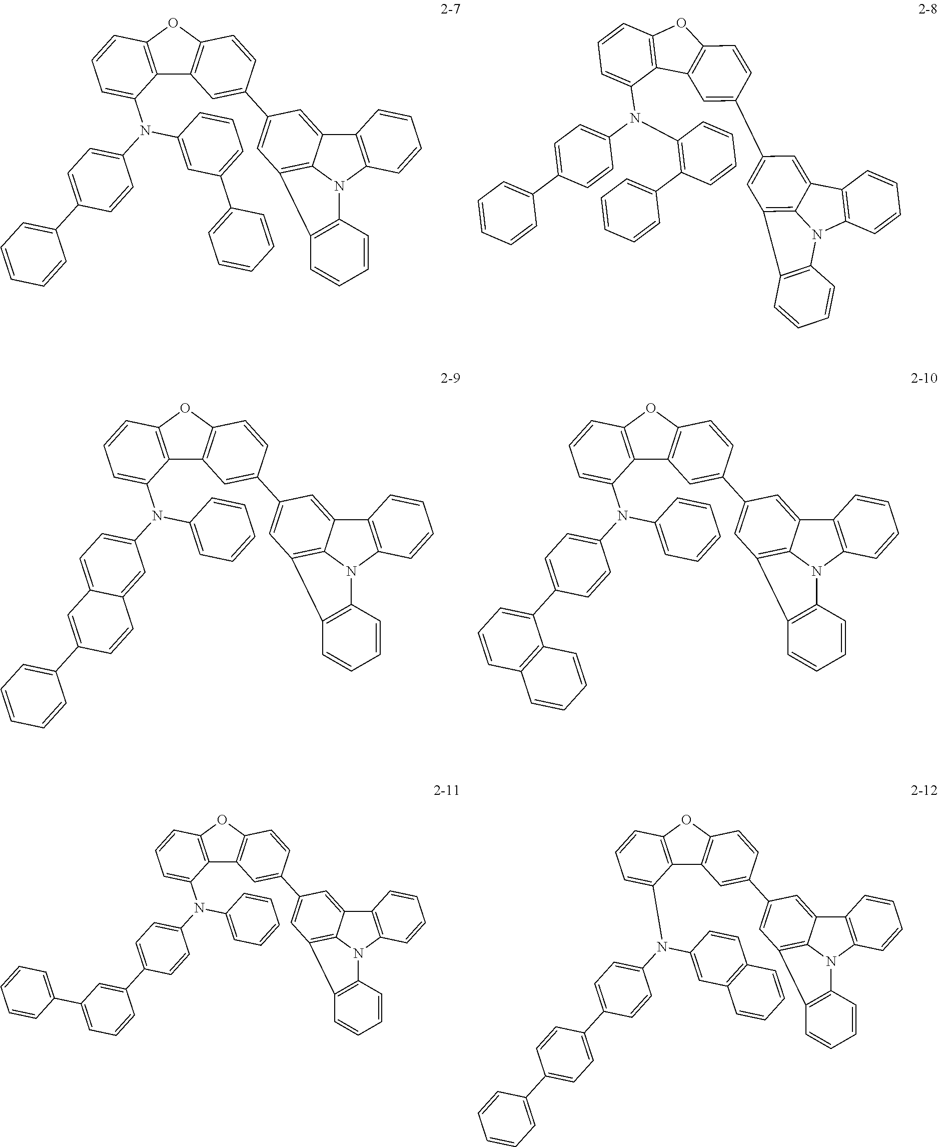

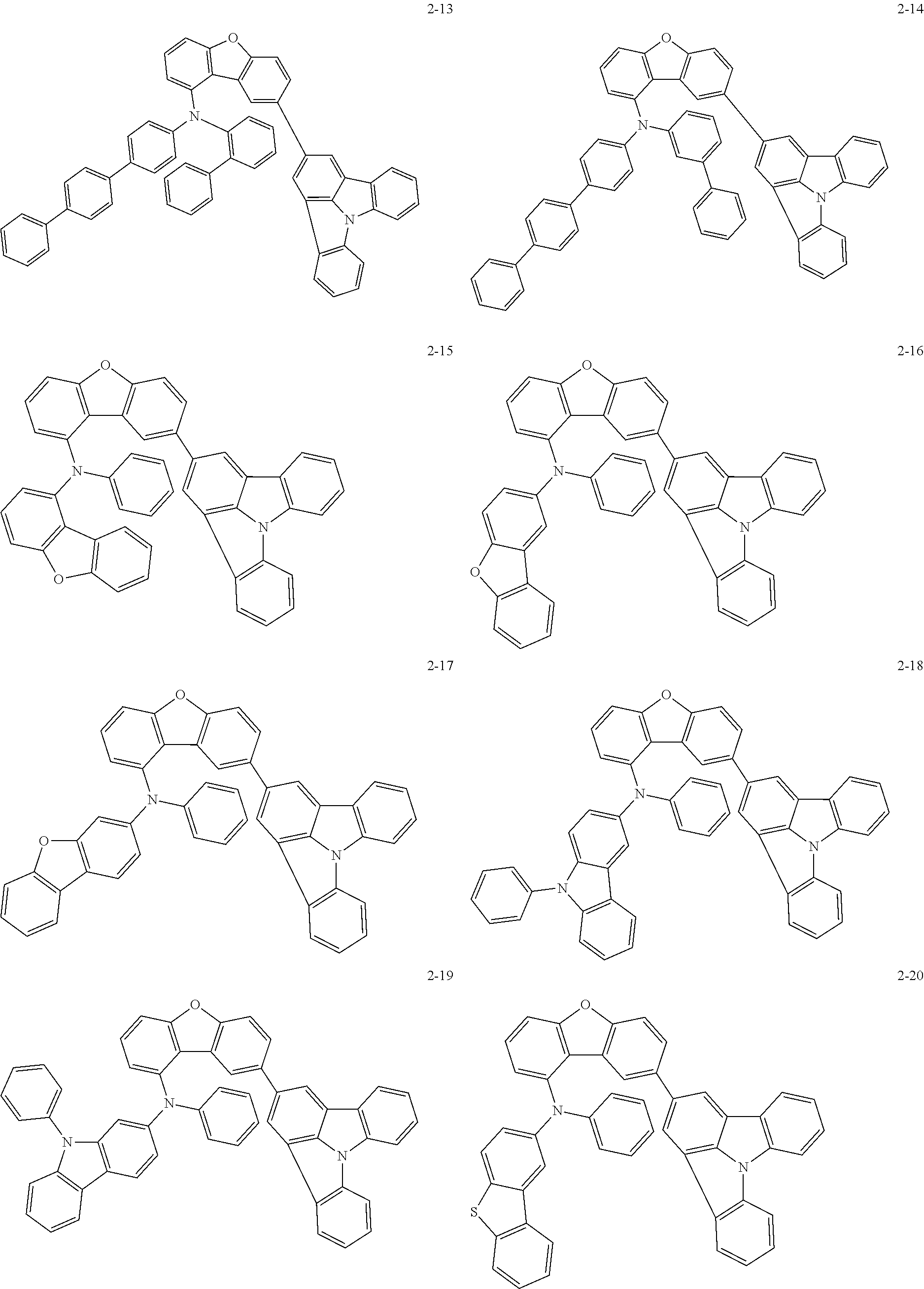

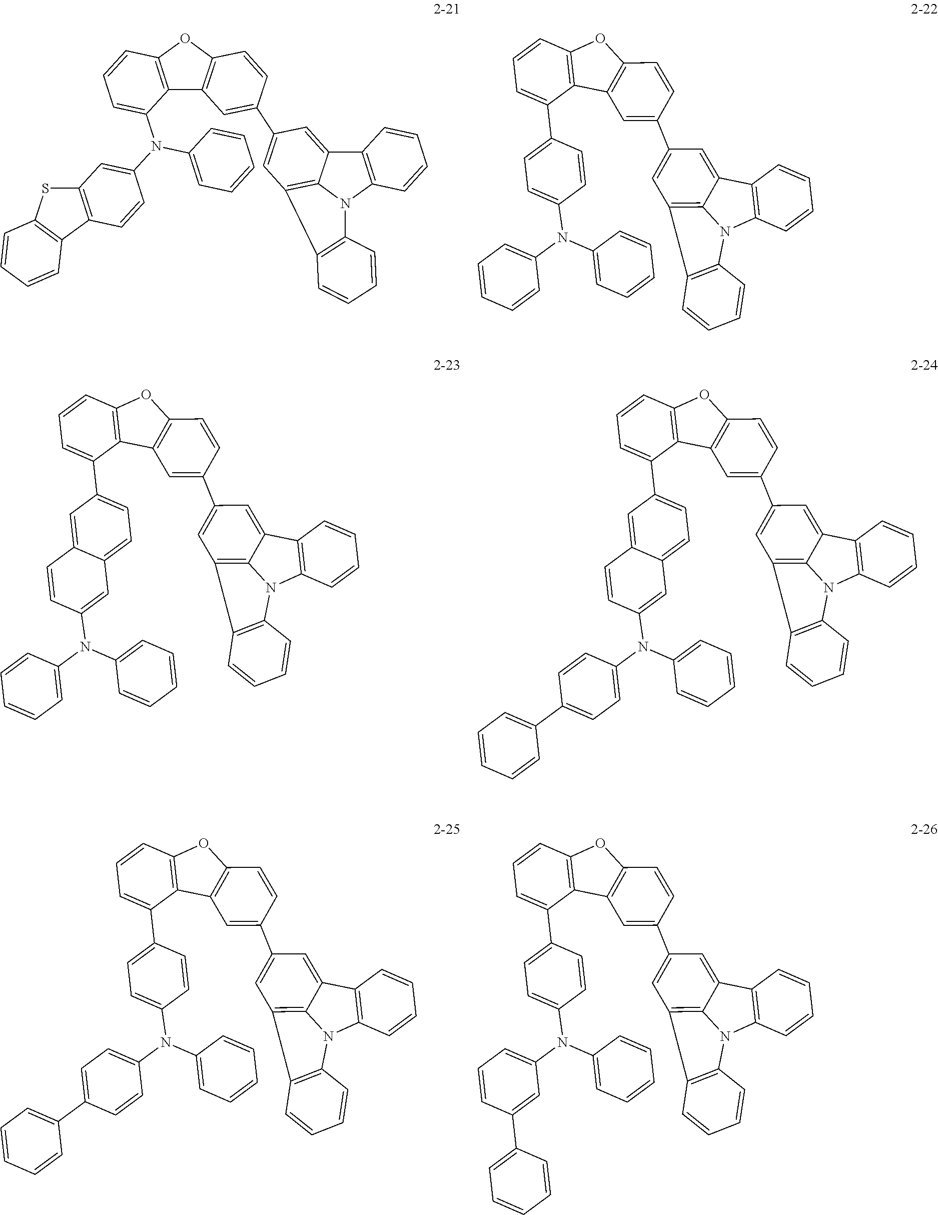

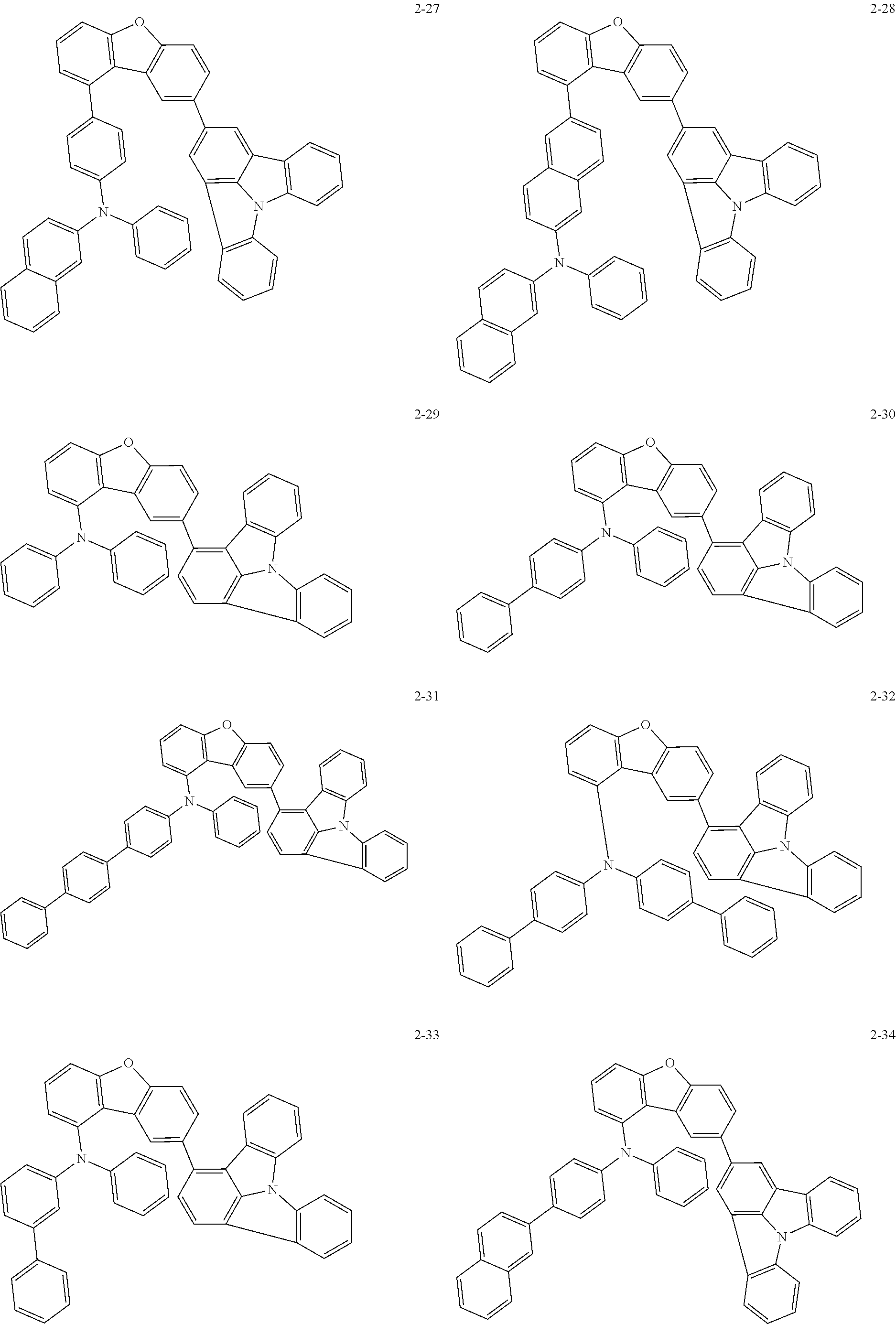

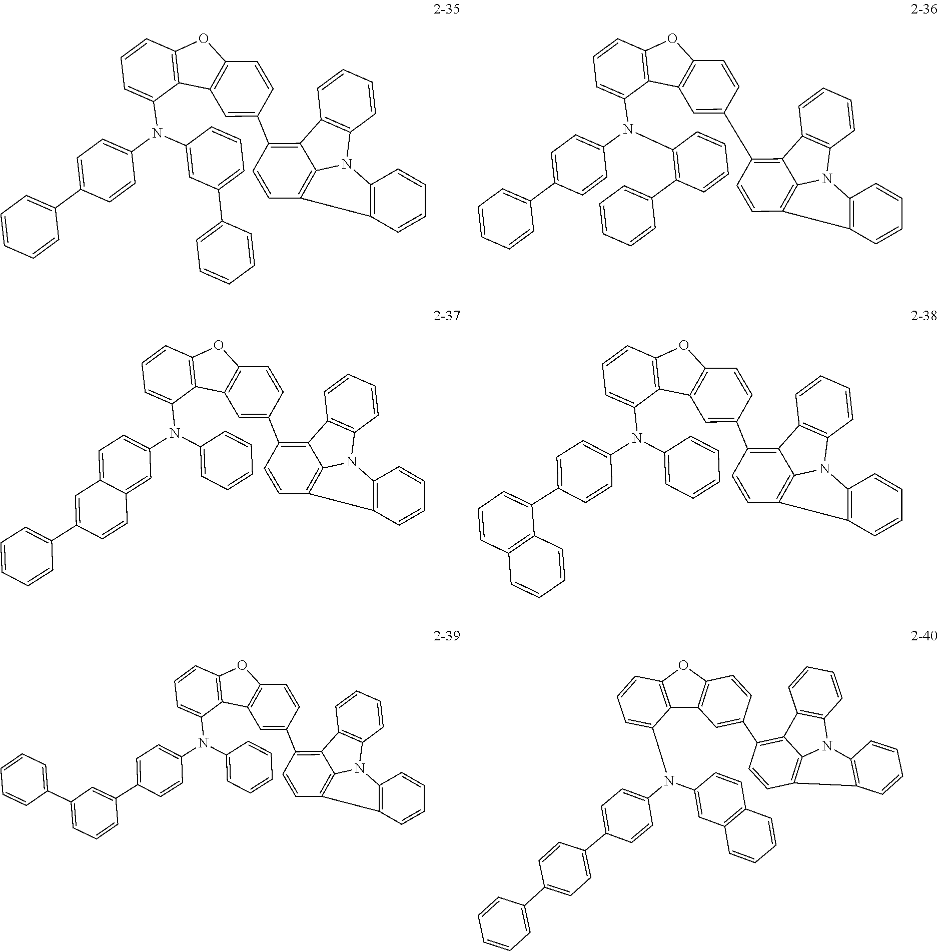

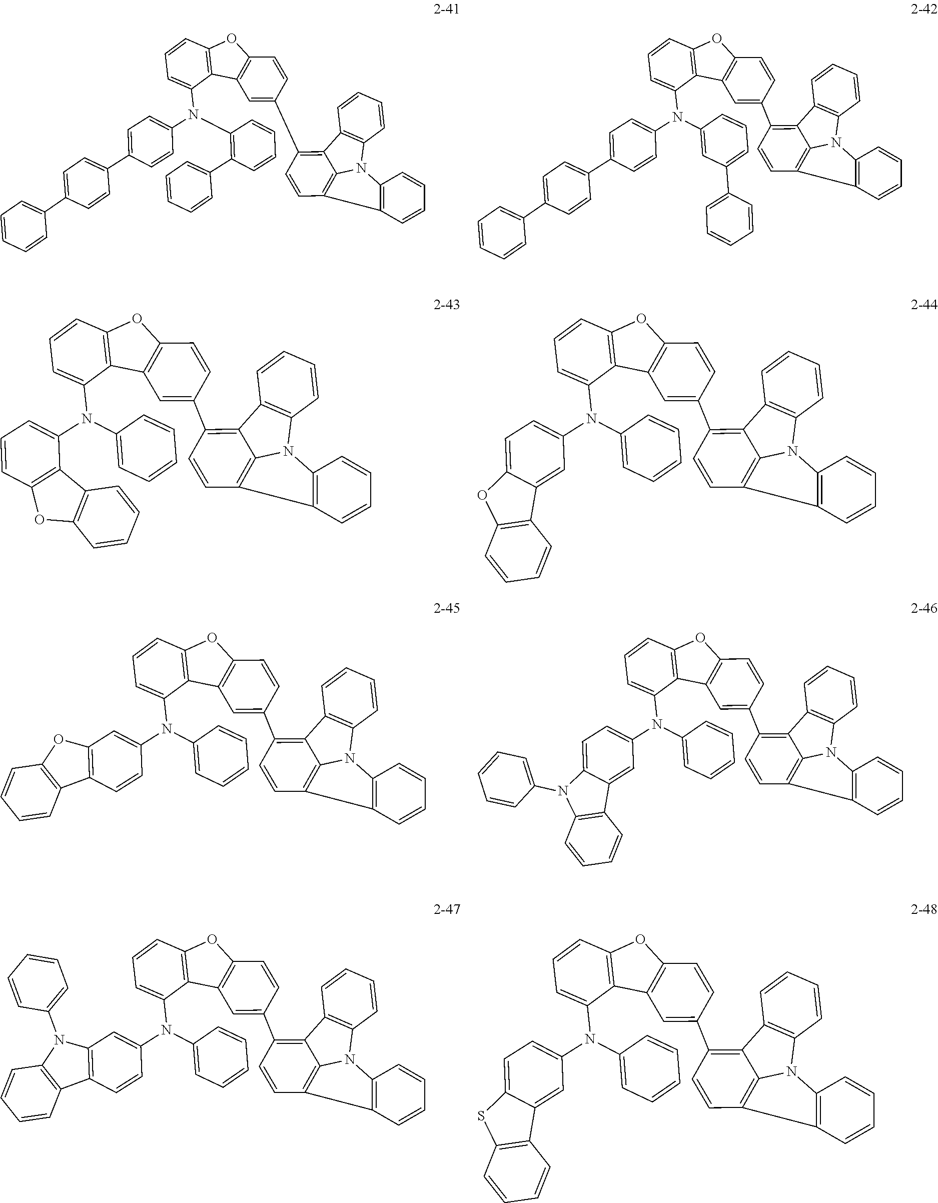

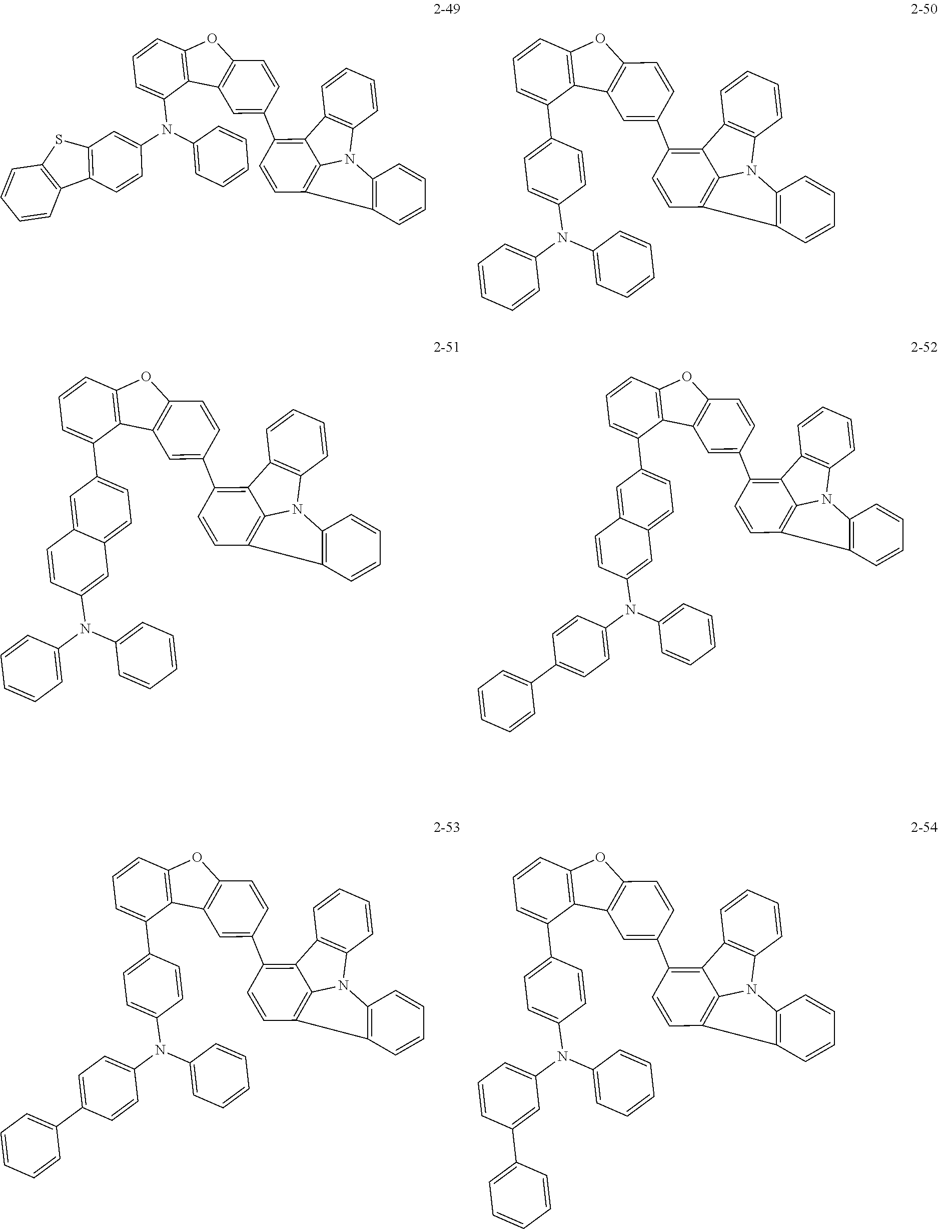

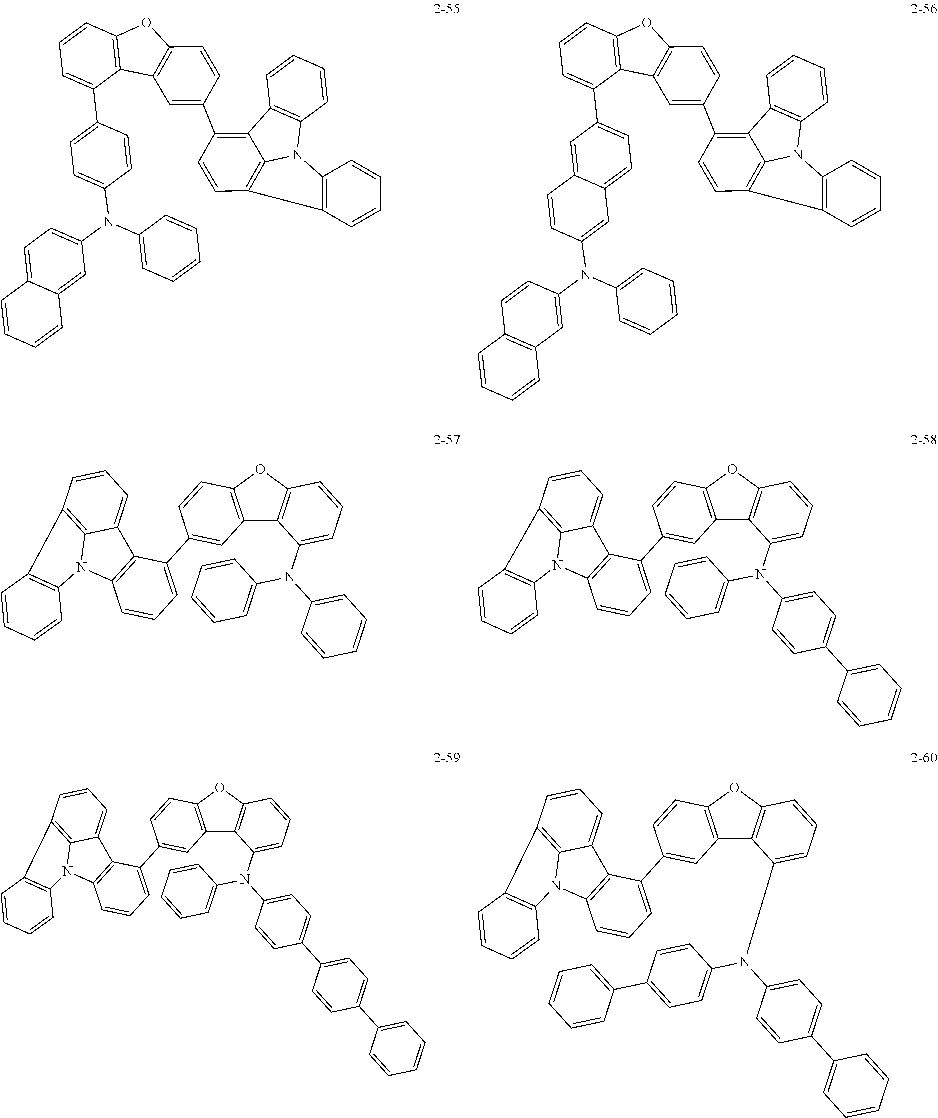

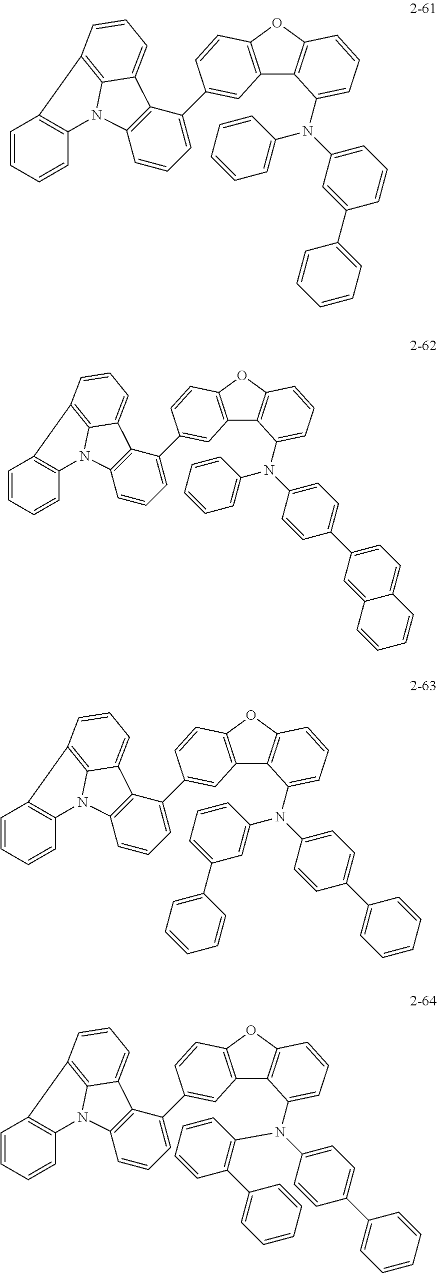 [SMILES: c1ccc(-c2cccc(N(c3ccccc3)c3cccc4oc5ccc(-c6cccc7c6c6cccc8c9ccccc9n7c86)cc5c34)c2)cc1 ; c1ccc(N(c2ccc(-c3ccc4ccccc4c3)cc2)c2cccc3oc4ccc(-c5cccc6c5c5cccc7c8ccccc8n6c75)cc4c23)cc1 ; c1ccc(-c2ccc(N(c3cccc(-c4ccccc4)c3)c3cccc4oc5ccc(-c6cccc7c6c6cccc8c9ccccc9n7c86)cc5c34)cc2)cc1 ; c1ccc(-c2ccc(N(c3ccccc3-c3ccccc3)c3cccc4oc5ccc(-c6cccc7c6c6cccc8c9ccccc9n7c86)cc5c34)cc2)cc1]

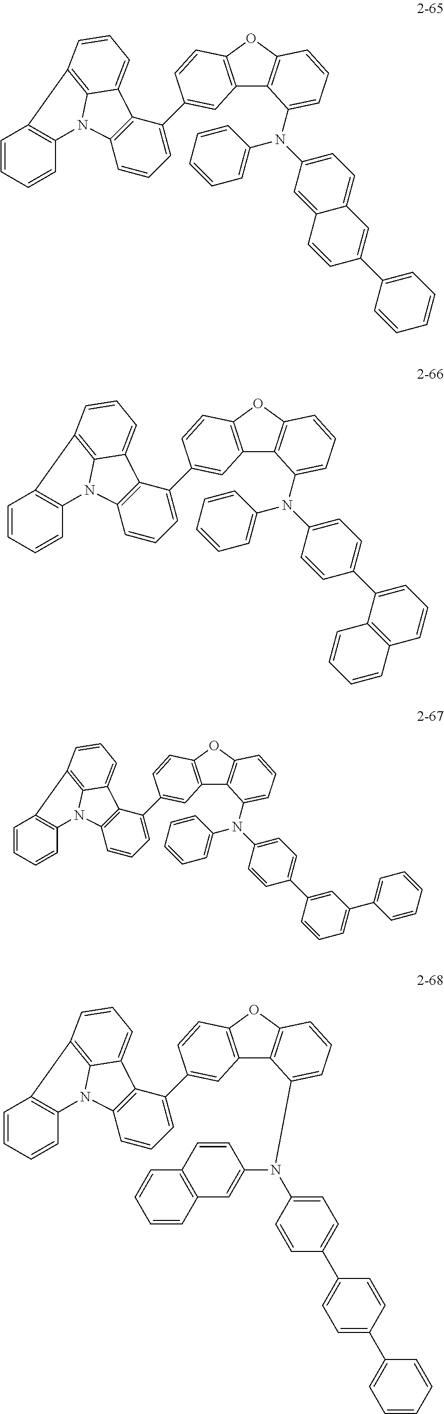 [SMILES: c1ccc(-c2ccc3cc(N(c4ccccc4)c4cccc5oc6ccc(-c7cccc8c7c7cccc9c%10ccccc%10n8c97)cc6c45)ccc3c2)cc1 ; c1ccc(N(c2ccc(-c3cccc4ccccc34)cc2)c2cccc3oc4ccc(-c5cccc6c5c5cccc7c8ccccc8n6c75)cc4c23)cc1 ; c1ccc(-c2cccc(-c3ccc(N(c4ccccc4)c4cccc5oc6ccc(-c7cccc8c7c7cccc9c%10ccccc%10n8c97)cc6c45)cc3)c2)cc1 ; c1ccc(-c2ccc(-c3ccc(N(c4ccc5ccccc5c4)c4cccc5oc6ccc(-c7cccc8c7c7cccc9c%10ccccc%10n8c97)cc6c45)cc3)cc2)cc1]

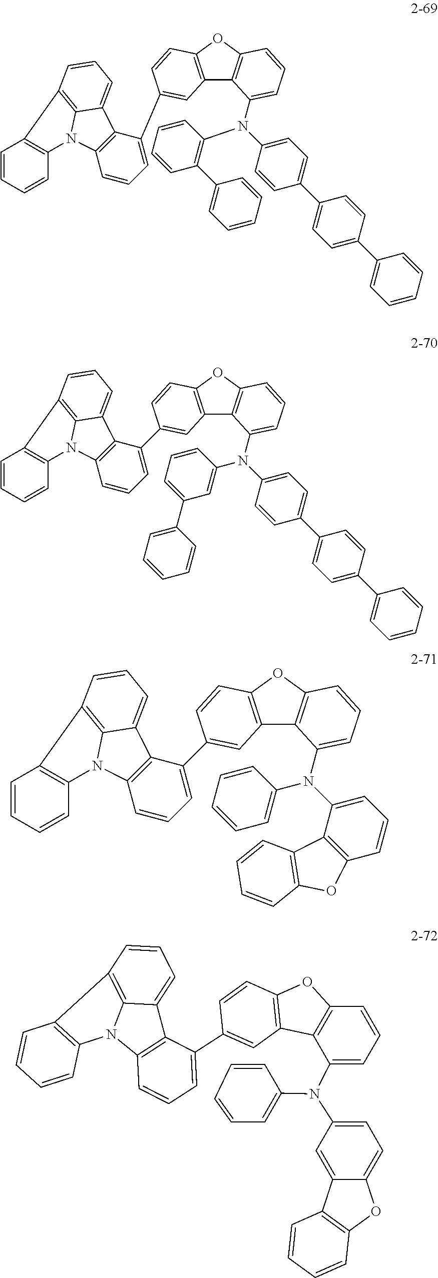 [SMILES: c1ccc(-c2ccc(-c3ccc(N(c4ccccc4-c4ccccc4)c4cccc5oc6ccc(-c7cccc8c7c7cccc9c%10ccccc%10n8c97)cc6c45)cc3)cc2)cc1 ; c1ccc(-c2ccc(-c3ccc(N(c4cccc(-c5ccccc5)c4)c4cccc5oc6ccc(-c7cccc8c7c7cccc9c%10ccccc%10n8c97)cc6c45)cc3)cc2)cc1 ; c1ccc(N(c2cccc3oc4ccccc4c23)c2cccc3oc4ccc(-c5cccc6c5c5cccc7c8ccccc8n6c75)cc4c23)cc1 ; c1ccc(N(c2ccc3oc4ccccc4c3c2)c2cccc3oc4ccc(-c5cccc6c5c5cccc7c8ccccc8n6c75)cc4c23)cc1]

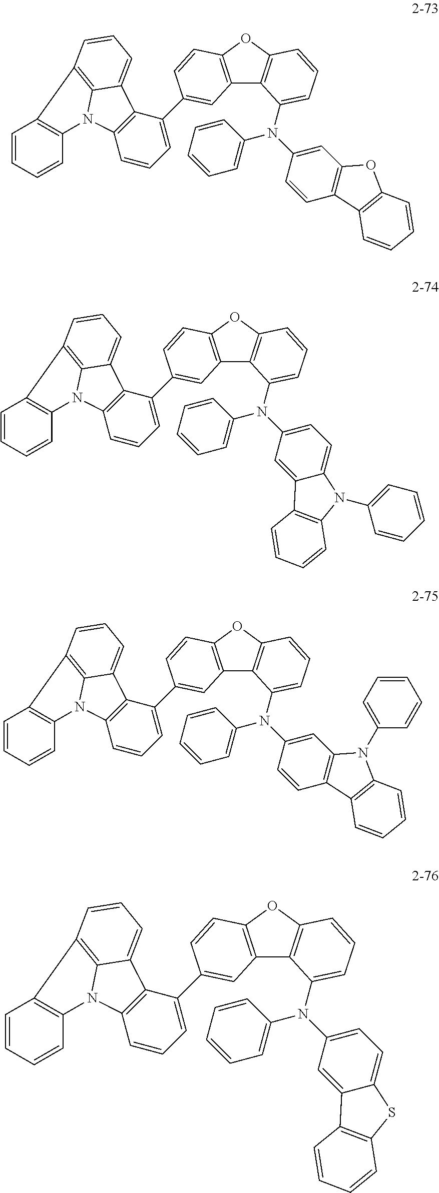 [SMILES: c1ccc(N(c2ccc3c(c2)oc2ccccc23)c2cccc3oc4ccc(-c5cccc6c5c5cccc7c8ccccc8n6c75)cc4c23)cc1 ; c1ccc(N(c2ccc3c(c2)c2ccccc2n3-c2ccccc2)c2cccc3oc4ccc(-c5cccc6c5c5cccc7c8ccccc8n6c75)cc4c23)cc1 ; c1ccc(N(c2ccc3c4ccccc4n(-c4ccccc4)c3c2)c2cccc3oc4ccc(-c5cccc6c5c5cccc7c8ccccc8n6c75)cc4c23)cc1 ; c1ccc(N(c2ccc3sc4ccccc4c3c2)c2cccc3oc4ccc(-c5cccc6c5c5cccc7c8ccccc8n6c75)cc4c23)cc1]

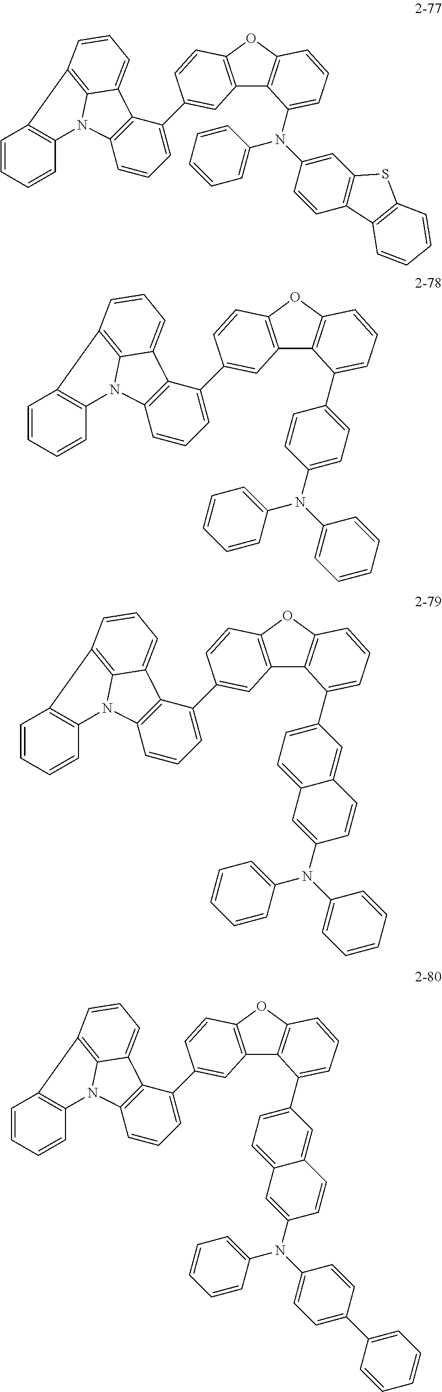 [SMILES: c1ccc(N(c2ccc3c(c2)sc2ccccc23)c2cccc3oc4ccc(-c5cccc6c5c5cccc7c8ccccc8n6c75)cc4c23)cc1 ; c1ccc(N(c2ccccc2)c2ccc(-c3cccc4oc5ccc(-c6cccc7c6c6cccc8c9ccccc9n7c86)cc5c34)cc2)cc1 ; c1ccc(N(c2ccccc2)c2ccc3cc(-c4cccc5oc6ccc(-c7cccc8c7c7cccc9c%10ccccc%10n8c97)cc6c45)ccc3c2)cc1 ; c1ccc(-c2ccc(N(c3ccccc3)c3ccc4cc(-c5cccc6oc7ccc(-c8cccc9c8c8cccc%10c%11ccccc%11n9c%108)cc7c56)ccc4c3)cc2)cc1]

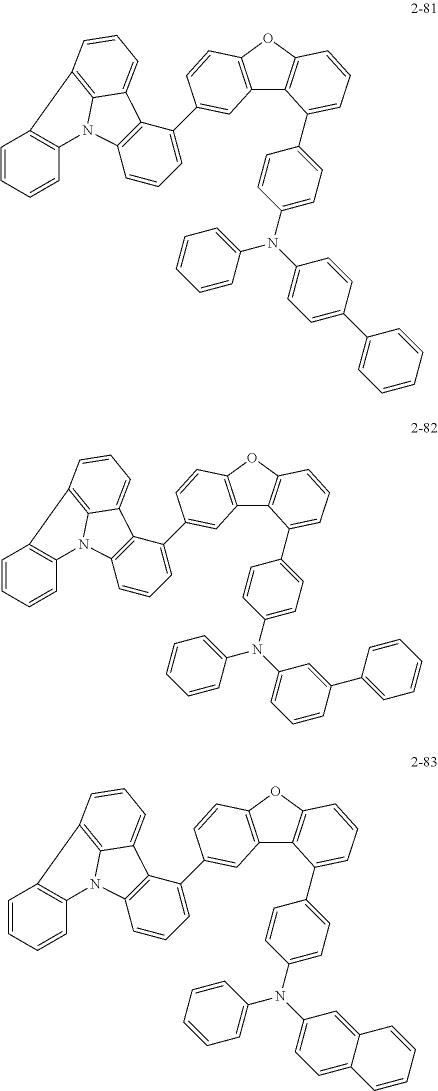 [SMILES: c1ccc(-c2ccc(N(c3ccccc3)c3ccc(-c4cccc5oc6ccc(-c7cccc8c7c7cccc9c%10ccccc%10n8c97)cc6c45)cc3)cc2)cc1 ; c1ccc(-c2cccc(N(c3ccccc3)c3ccc(-c4cccc5oc6ccc(-c7cccc8c7c7cccc9c%10ccccc%10n8c97)cc6c45)cc3)c2)cc1 ; c1ccc(N(c2ccc(-c3cccc4oc5ccc(-c6cccc7c6c6cccc8c9ccccc9n7c86)cc5c34)cc2)c2ccc3ccccc3c2)cc1]

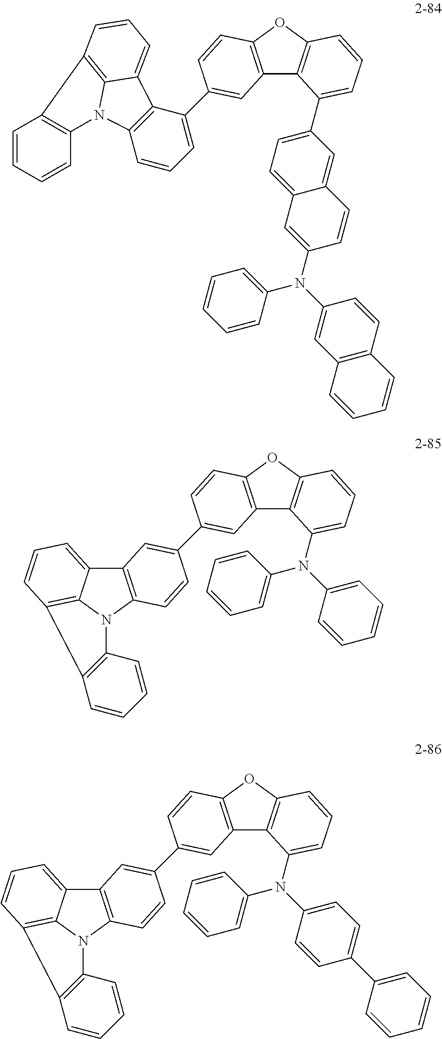 [SMILES: c1ccc(N(c2ccc3ccccc3c2)c2ccc3cc(-c4cccc5oc6ccc(-c7cccc8c7c7cccc9c%10ccccc%10n8c97)cc6c45)ccc3c2)cc1 ; c1ccc(N(c2ccccc2)c2cccc3oc4ccc(-c5ccc6c(c5)c5cccc7c8ccccc8n6c75)cc4c23)cc1 ; c1ccc(-c2ccc(N(c3ccccc3)c3cccc4oc5ccc(-c6ccc7c(c6)c6cccc8c9ccccc9n7c86)cc5c34)cc2)cc1]

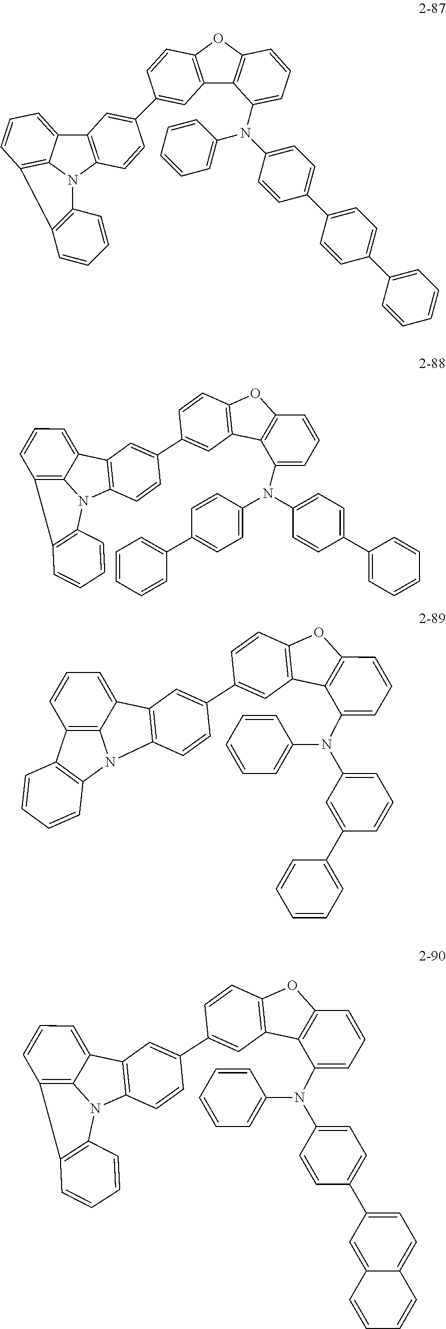 [SMILES: c1ccc(-c2ccc(-c3ccc(N(c4ccccc4)c4cccc5oc6ccc(-c7ccc8c(c7)c7cccc9c%10ccccc%10n8c97)cc6c45)cc3)cc2)cc1 ; c1ccc(-c2ccc(N(c3ccc(-c4ccccc4)cc3)c3cccc4oc5ccc(-c6ccc7c(c6)c6cccc8c9ccccc9n7c86)cc5c34)cc2)cc1 ; c1ccc(-c2cccc(N(c3ccccc3)c3cccc4oc5ccc(-c6ccc7c(c6)c6cccc8c9ccccc9n7c86)cc5c34)c2)cc1 ; c1ccc(N(c2ccc(-c3ccc4ccccc4c3)cc2)c2cccc3oc4ccc(-c5ccc6c(c5)c5cccc7c8ccccc8n6c75)cc4c23)cc1]

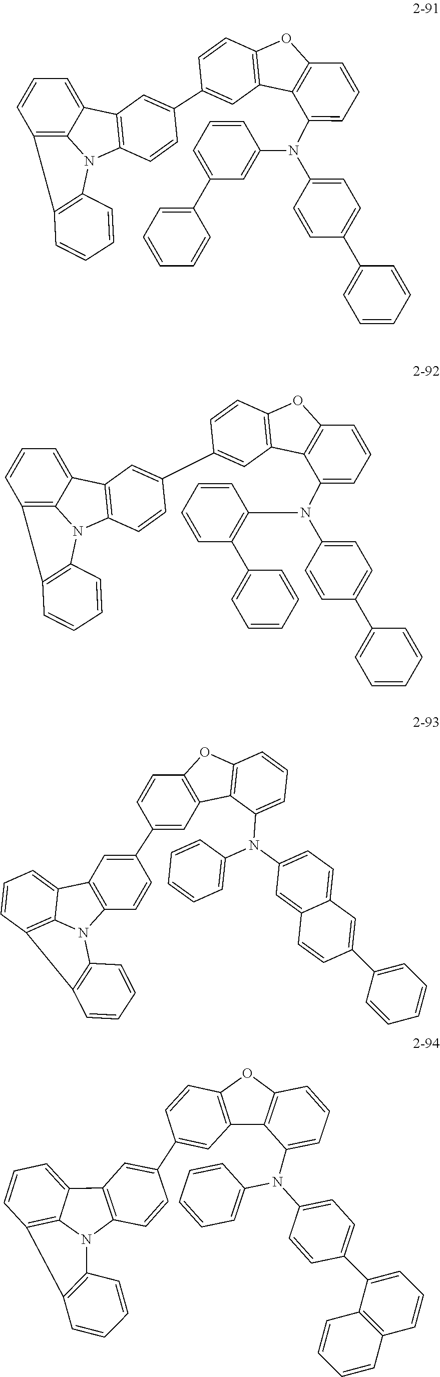 [SMILES: c1ccc(-c2ccc(N(c3cccc(-c4ccccc4)c3)c3cccc4oc5ccc(-c6ccc7c(c6)c6cccc8c9ccccc9n7c86)cc5c34)cc2)cc1 ; c1ccc(-c2ccc(N(c3ccccc3-c3ccccc3)c3cccc4oc5ccc(-c6ccc7c(c6)c6cccc8c9ccccc9n7c86)cc5c34)cc2)cc1 ; c1ccc(-c2ccc3cc(N(c4ccccc4)c4cccc5oc6ccc(-c7ccc8c(c7)c7cccc9c%10ccccc%10n8c97)cc6c45)ccc3c2)cc1 ; c1ccc(N(c2ccc(-c3cccc4ccccc34)cc2)c2cccc3oc4ccc(-c5ccc6c(c5)c5cccc7c8ccccc8n6c75)cc4c23)cc1]

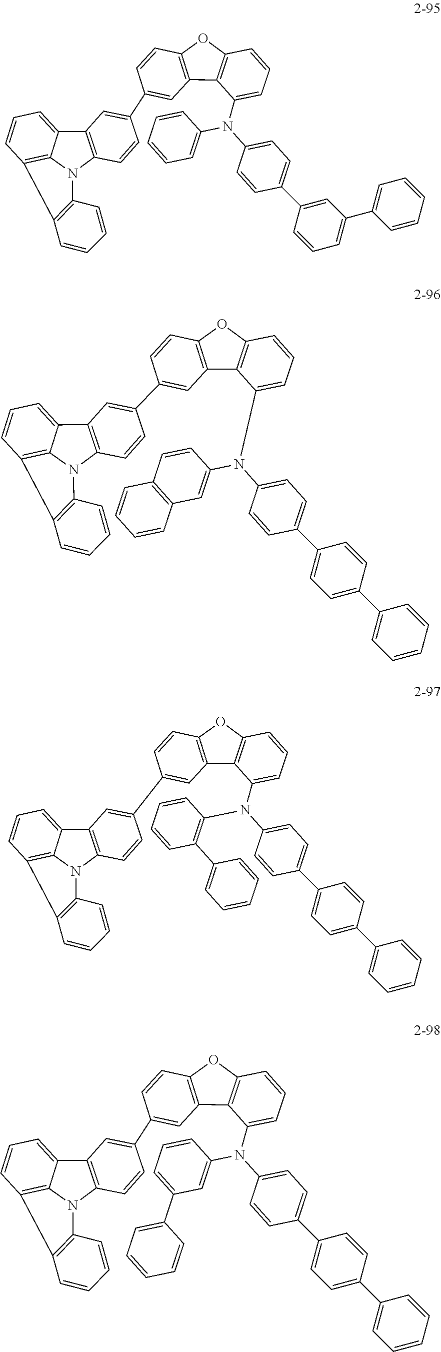 [SMILES: c1ccc(-c2cccc(-c3ccc(N(c4ccccc4)c4cccc5oc6ccc(-c7ccc8c(c7)c7cccc9c%10ccccc%10n8c97)cc6c45)cc3)c2)cc1 ; c1ccc(-c2ccc(-c3ccc(N(c4ccc5ccccc5c4)c4cccc5oc6ccc(-c7ccc8c(c7)c7cccc9c%10ccccc%10n8c97)cc6c45)cc3)cc2)cc1 ; c1ccc(-c2ccc(-c3ccc(N(c4ccccc4-c4ccccc4)c4cccc5oc6ccc(-c7ccc8c(c7)c7cccc9c%10ccccc%10n8c97)cc6c45)cc3)cc2)cc1 ; c1ccc(-c2ccc(-c3ccc(N(c4cccc(-c5ccccc5)c4)c4cccc5oc6ccc(-c7ccc8c(c7)c7cccc9c%10ccccc%10n8c97)cc6c45)cc3)cc2)cc1]

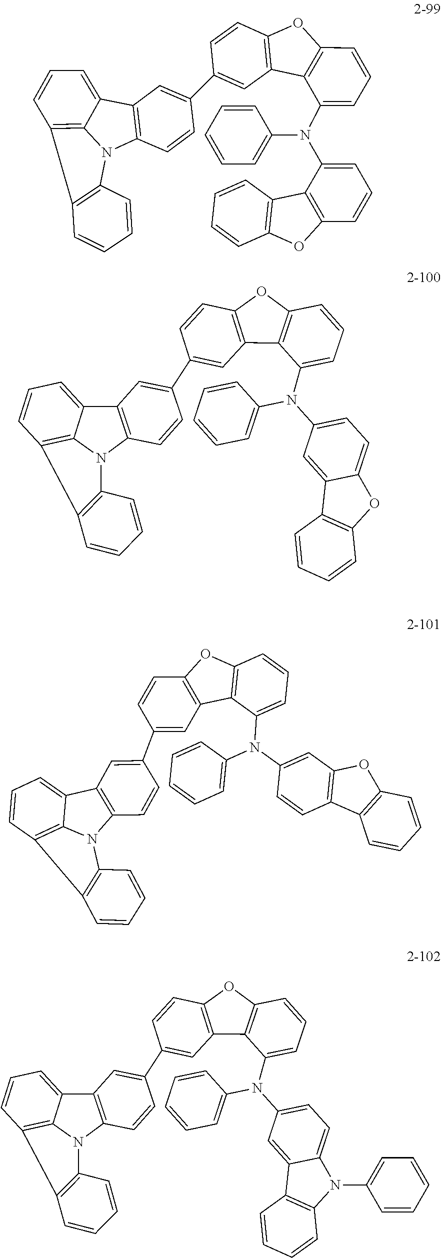 [SMILES: c1ccc(N(c2cccc3oc4ccccc4c23)c2cccc3oc4ccc(-c5ccc6c(c5)c5cccc7c8ccccc8n6c75)cc4c23)cc1 ; c1ccc(N(c2ccc3oc4ccccc4c3c2)c2cccc3oc4ccc(-c5ccc6c(c5)c5cccc7c8ccccc8n6c75)cc4c23)cc1 ; c1ccc(N(c2ccc3c(c2)oc2ccccc23)c2cccc3oc4ccc(-c5ccc6c(c5)c5cccc7c8ccccc8n6c75)cc4c23)cc1 ; c1ccc(N(c2ccc3c(c2)c2ccccc2n3-c2ccccc2)c2cccc3oc4ccc(-c5ccc6c(c5)c5cccc7c8ccccc8n6c75)cc4c23)cc1]

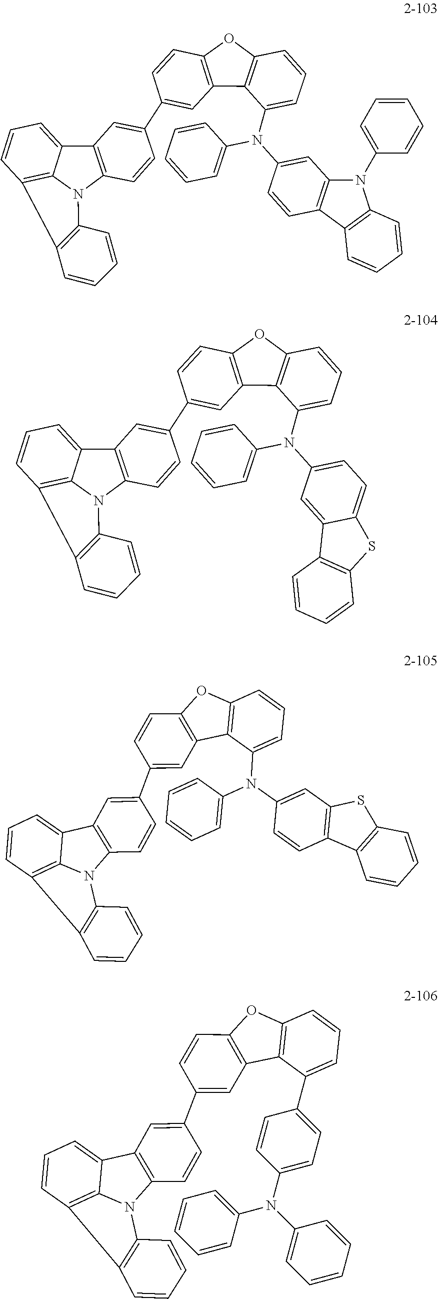 [SMILES: c1ccc(N(c2ccc3c4ccccc4n(-c4ccccc4)c3c2)c2cccc3oc4ccc(-c5ccc6c(c5)c5cccc7c8ccccc8n6c75)cc4c23)cc1 ; c1ccc(N(c2ccc3sc4ccccc4c3c2)c2cccc3oc4ccc(-c5ccc6c(c5)c5cccc7c8ccccc8n6c75)cc4c23)cc1 ; c1ccc(N(c2ccc3c(c2)sc2ccccc23)c2cccc3oc4ccc(-c5ccc6c(c5)c5cccc7c8ccccc8n6c75)cc4c23)cc1 ; c1ccc(N(c2ccccc2)c2ccc(-c3cccc4oc5ccc(-c6ccc7c(c6)c6cccc8c9ccccc9n7c86)cc5c34)cc2)cc1]

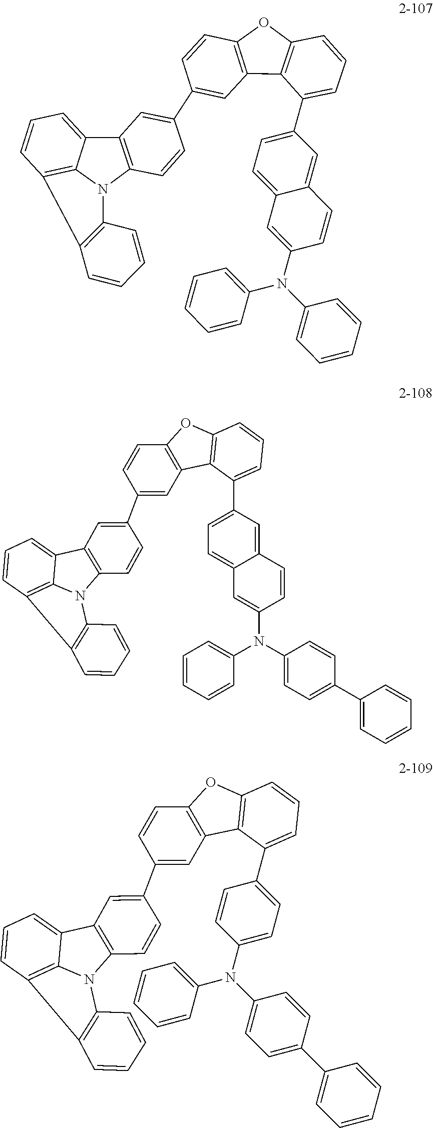 [SMILES: c1ccc(N(c2ccccc2)c2ccc3cc(-c4cccc5oc6ccc(-c7ccc8c(c7)c7cccc9c%10ccccc%10n8c97)cc6c45)ccc3c2)cc1 ; c1ccc(-c2ccc(N(c3ccccc3)c3ccc4cc(-c5cccc6oc7ccc(-c8ccc9c(c8)c8cccc%10c%11ccccc%11n9c%108)cc7c56)ccc4c3)cc2)cc1 ; c1ccc(-c2ccc(N(c3ccccc3)c3ccc(-c4cccc5oc6ccc(-c7ccc8c(c7)c7cccc9c%10ccccc%10n8c97)cc6c45)cc3)cc2)cc1]

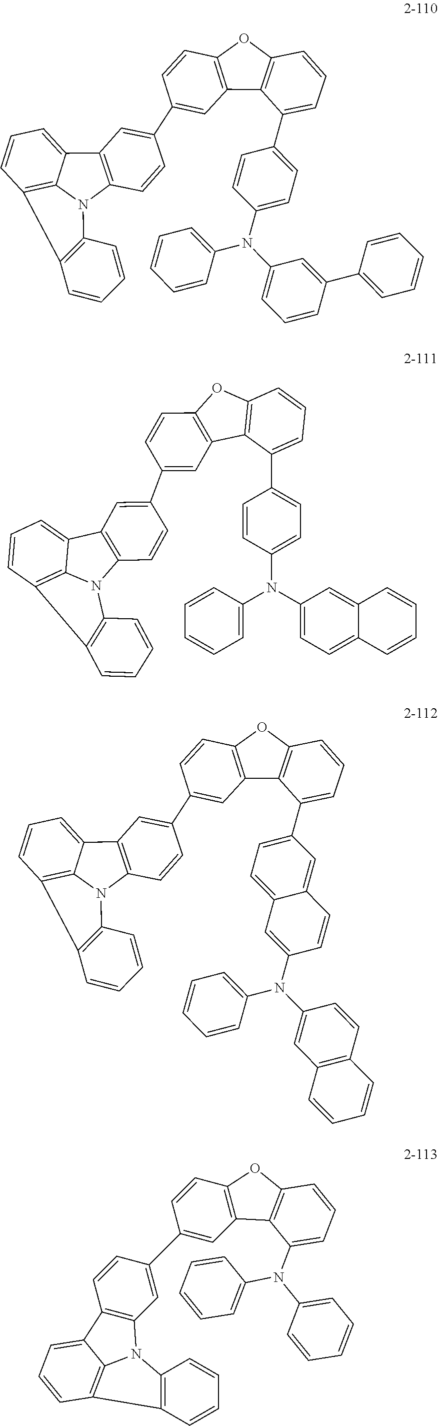 [SMILES: c1ccc(-c2cccc(N(c3ccccc3)c3ccc(-c4cccc5oc6ccc(-c7ccc8c(c7)c7cccc9c%10ccccc%10n8c97)cc6c45)cc3)c2)cc1 ; c1ccc(N(c2ccc(-c3cccc4oc5ccc(-c6ccc7c(c6)c6cccc8c9ccccc9n7c86)cc5c34)cc2)c2ccc3ccccc3c2)cc1 ; c1ccc(N(c2ccc3ccccc3c2)c2ccc3cc(-c4cccc5oc6ccc(-c7ccc8c(c7)c7cccc9c%10ccccc%10n8c97)cc6c45)ccc3c2)cc1 ; c1ccc(N(c2ccccc2)c2cccc3oc4ccc(-c5ccc6c7cccc8c9ccccc9n(c6c5)c87)cc4c23)cc1]

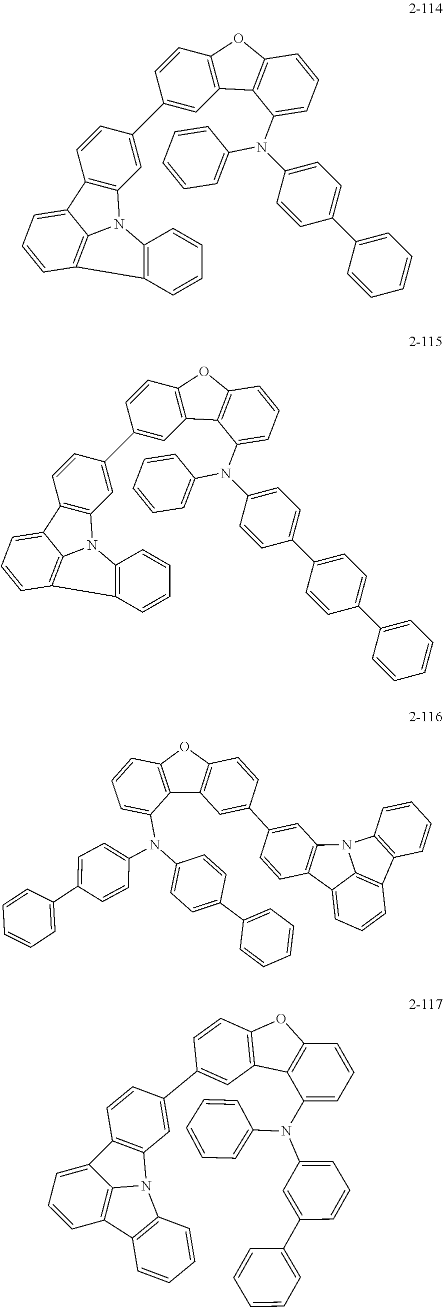 [SMILES: c1ccc(-c2ccc(N(c3ccccc3)c3cccc4oc5ccc(-c6ccc7c8cccc9c%10ccccc%10n(c7c6)c98)cc5c34)cc2)cc1 ; c1ccc(-c2ccc(-c3ccc(N(c4ccccc4)c4cccc5oc6ccc(-c7ccc8c9cccc%10c%11ccccc%11n(c8c7)c%109)cc6c45)cc3)cc2)cc1 ; c1ccc(-c2ccc(N(c3ccc(-c4ccccc4)cc3)c3cccc4oc5ccc(-c6ccc7c8cccc9c%10ccccc%10n(c7c6)c98)cc5c34)cc2)cc1 ; c1ccc(-c2cccc(N(c3ccccc3)c3cccc4oc5ccc(-c6ccc7c8cccc9c%10ccccc%10n(c7c6)c98)cc5c34)c2)cc1]

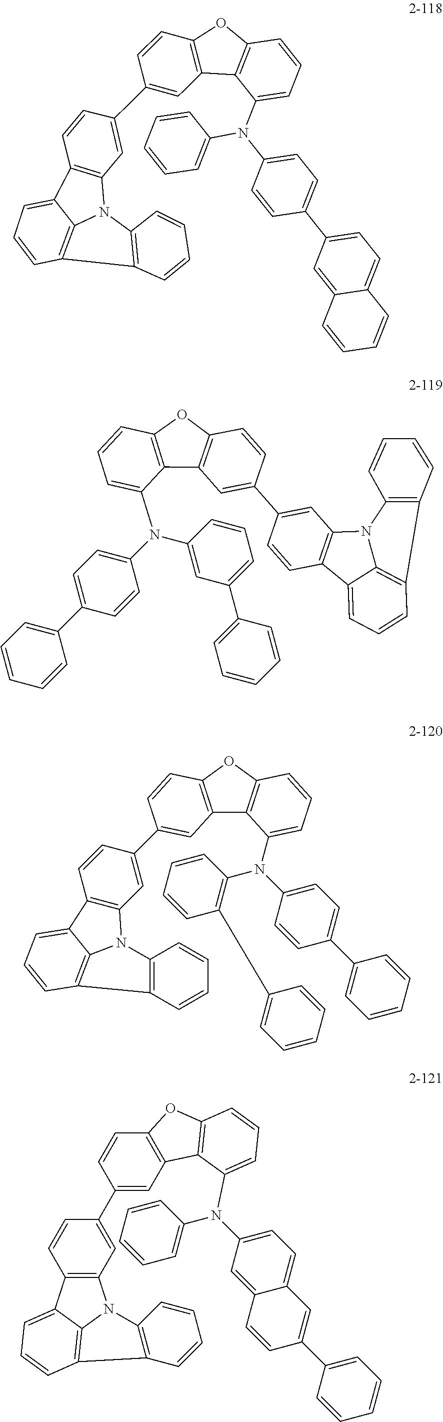 [SMILES: c1ccc(N(c2ccc(-c3ccc4ccccc4c3)cc2)c2cccc3oc4ccc(-c5ccc6c7cccc8c9ccccc9n(c6c5)c87)cc4c23)cc1 ; c1ccc(-c2ccc(N(c3cccc(-c4ccccc4)c3)c3cccc4oc5ccc(-c6ccc7c8cccc9c%10ccccc%10n(c7c6)c98)cc5c34)cc2)cc1 ; c1ccc(-c2ccc(N(c3ccccc3-c3ccccc3)c3cccc4oc5ccc(-c6ccc7c8cccc9c%10ccccc%10n(c7c6)c98)cc5c34)cc2)cc1 ; c1ccc(-c2ccc3cc(N(c4ccccc4)c4cccc5oc6ccc(-c7ccc8c9cccc%10c%11ccccc%11n(c8c7)c%109)cc6c45)ccc3c2)cc1]

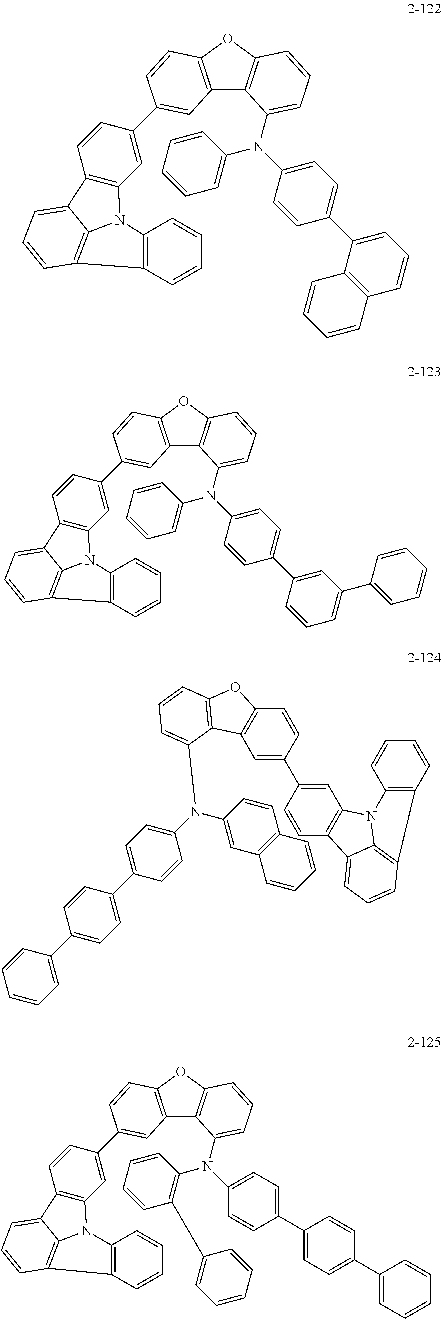 [SMILES: c1ccc(N(c2ccc(-c3cccc4ccccc34)cc2)c2cccc3oc4ccc(-c5ccc6c7cccc8c9ccccc9n(c6c5)c87)cc4c23)cc1 ; c1ccc(-c2cccc(-c3ccc(N(c4ccccc4)c4cccc5oc6ccc(-c7ccc8c9cccc%10c%11ccccc%11n(c8c7)c%109)cc6c45)cc3)c2)cc1 ; c1ccc(-c2ccc(-c3ccc(N(c4ccc5ccccc5c4)c4cccc5oc6ccc(-c7ccc8c9cccc%10c%11ccccc%11n(c8c7)c%109)cc6c45)cc3)cc2)cc1 ; c1ccc(-c2ccc(-c3ccc(N(c4ccccc4-c4ccccc4)c4cccc5oc6ccc(-c7ccc8c9cccc%10c%11ccccc%11n(c8c7)c%109)cc6c45)cc3)cc2)cc1]

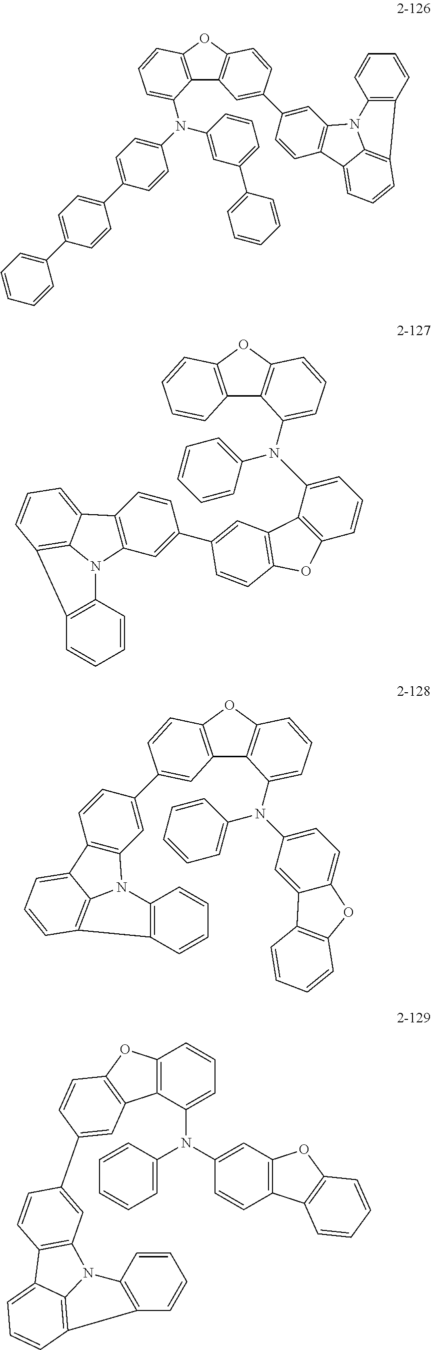 [SMILES: c1ccc(-c2ccc(-c3ccc(N(c4cccc(-c5ccccc5)c4)c4cccc5oc6ccc(-c7ccc8c9cccc%10c%11ccccc%11n(c8c7)c%109)cc6c45)cc3)cc2)cc1 ; c1ccc(N(c2cccc3oc4ccccc4c23)c2cccc3oc4ccc(-c5ccc6c7cccc8c9ccccc9n(c6c5)c87)cc4c23)cc1 ; c1ccc(N(c2ccc3oc4ccccc4c3c2)c2cccc3oc4ccc(-c5ccc6c7cccc8c9ccccc9n(c6c5)c87)cc4c23)cc1 ; c1ccc(N(c2ccc3c(c2)oc2ccccc23)c2cccc3oc4ccc(-c5ccc6c7cccc8c9ccccc9n(c6c5)c87)cc4c23)cc1]

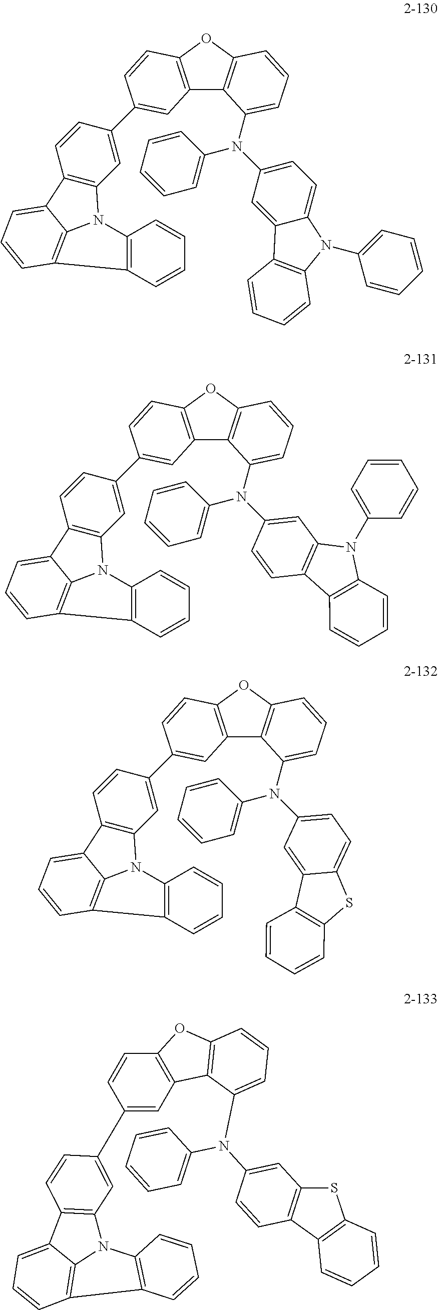 [SMILES: c1ccc(N(c2ccc3c(c2)c2ccccc2n3-c2ccccc2)c2cccc3oc4ccc(-c5ccc6c7cccc8c9ccccc9n(c6c5)c87)cc4c23)cc1 ; c1ccc(N(c2ccc3c4ccccc4n(-c4ccccc4)c3c2)c2cccc3oc4ccc(-c5ccc6c7cccc8c9ccccc9n(c6c5)c87)cc4c23)cc1 ; c1ccc(N(c2ccc3sc4ccccc4c3c2)c2cccc3oc4ccc(-c5ccc6c7cccc8c9ccccc9n(c6c5)c87)cc4c23)cc1 ; c1ccc(N(c2ccc3c(c2)sc2ccccc23)c2cccc3oc4ccc(-c5ccc6c7cccc8c9ccccc9n(c6c5)c87)cc4c23)cc1]

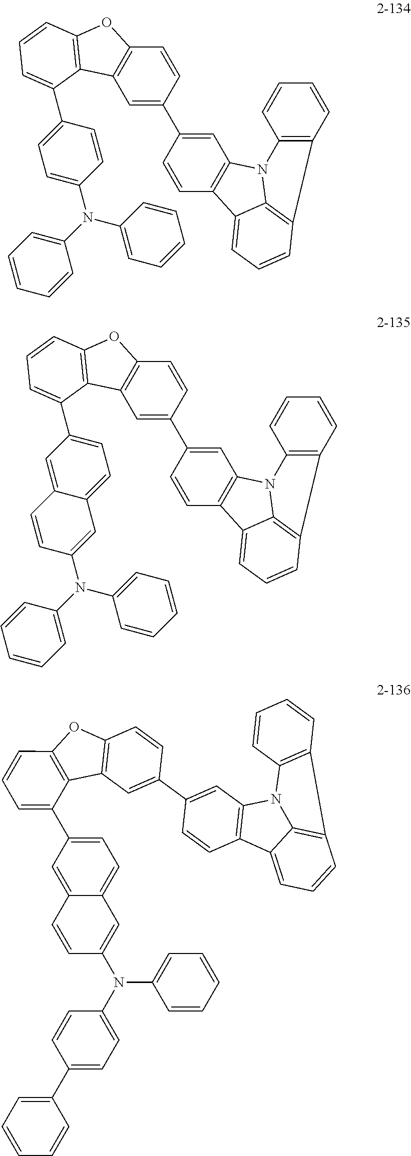

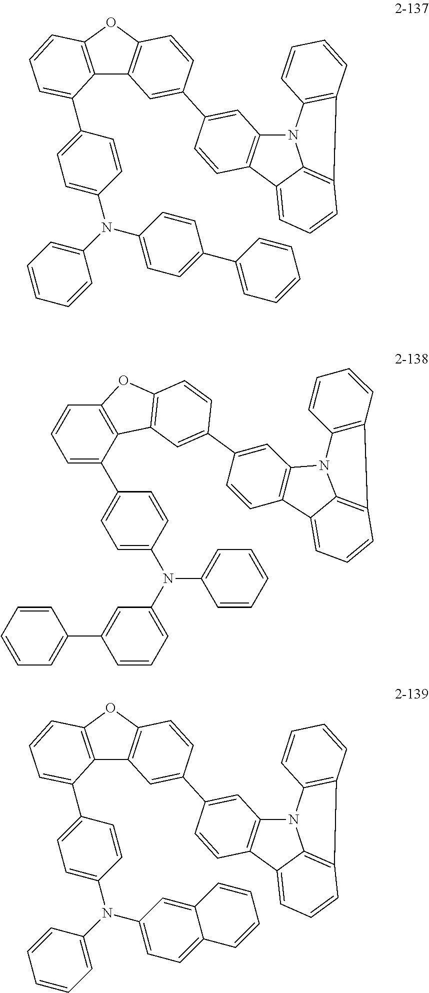

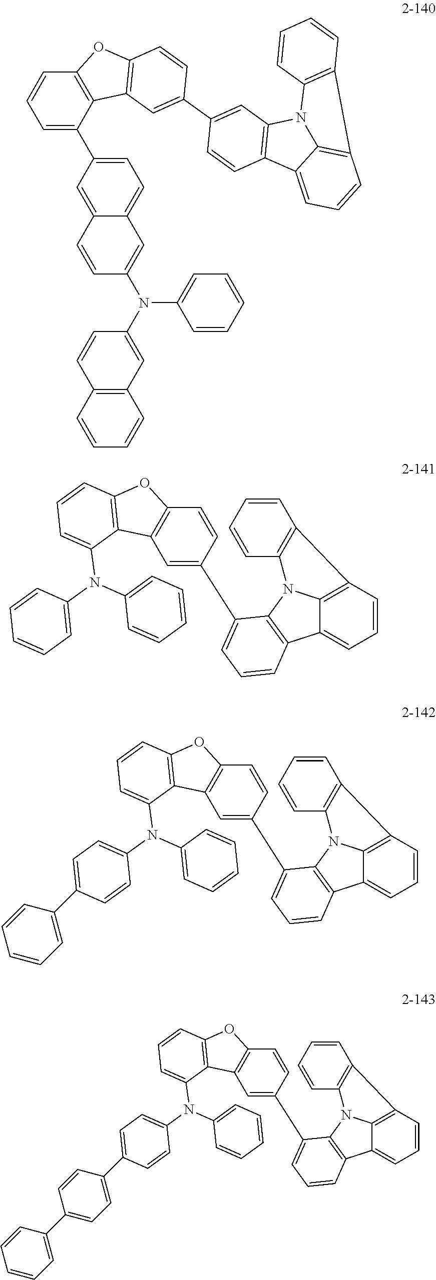

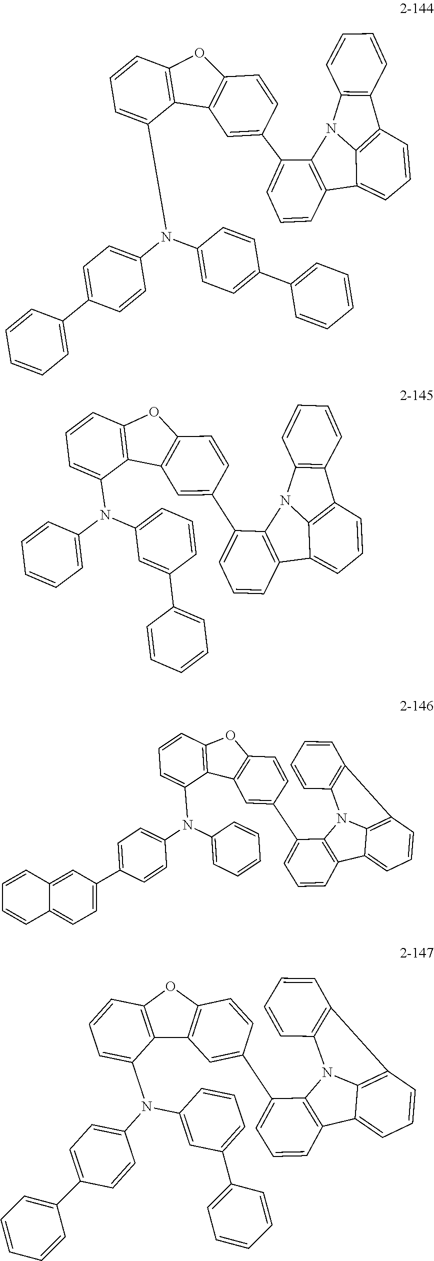 [SMILES: c1ccc(-c2ccc(N(c3ccc(-c4ccccc4)cc3)c3cccc4oc5ccc(-c6cccc7c8cccc9c%10ccccc%10n(c67)c98)cc5c34)cc2)cc1 ; c1ccc(-c2cccc(N(c3ccccc3)c3cccc4oc5ccc(-c6cccc7c8cccc9c%10ccccc%10n(c67)c98)cc5c34)c2)cc1 ; c1ccc(N(c2ccc(-c3ccc4ccccc4c3)cc2)c2cccc3oc4ccc(-c5cccc6c7cccc8c9ccccc9n(c56)c87)cc4c23)cc1 ; c1ccc(-c2ccc(N(c3cccc(-c4ccccc4)c3)c3cccc4oc5ccc(-c6cccc7c8cccc9c%10ccccc%10n(c67)c98)cc5c34)cc2)cc1]

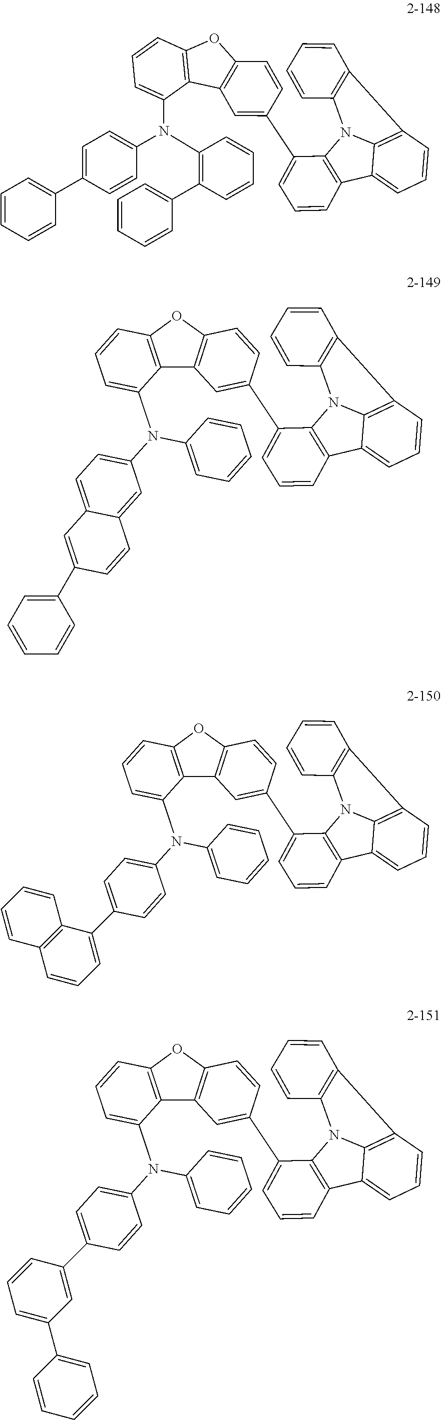 [SMILES: c1ccc(-c2ccc(N(c3ccccc3-c3ccccc3)c3cccc4oc5ccc(-c6cccc7c8cccc9c%10ccccc%10n(c67)c98)cc5c34)cc2)cc1 ; c1ccc(-c2ccc3cc(N(c4ccccc4)c4cccc5oc6ccc(-c7cccc8c9cccc%10c%11ccccc%11n(c78)c%109)cc6c45)ccc3c2)cc1 ; c1ccc(N(c2ccc(-c3cccc4ccccc34)cc2)c2cccc3oc4ccc(-c5cccc6c7cccc8c9ccccc9n(c56)c87)cc4c23)cc1 ; c1ccc(-c2cccc(-c3ccc(N(c4ccccc4)c4cccc5oc6ccc(-c7cccc8c9cccc%10c%11ccccc%11n(c78)c%109)cc6c45)cc3)c2)cc1]

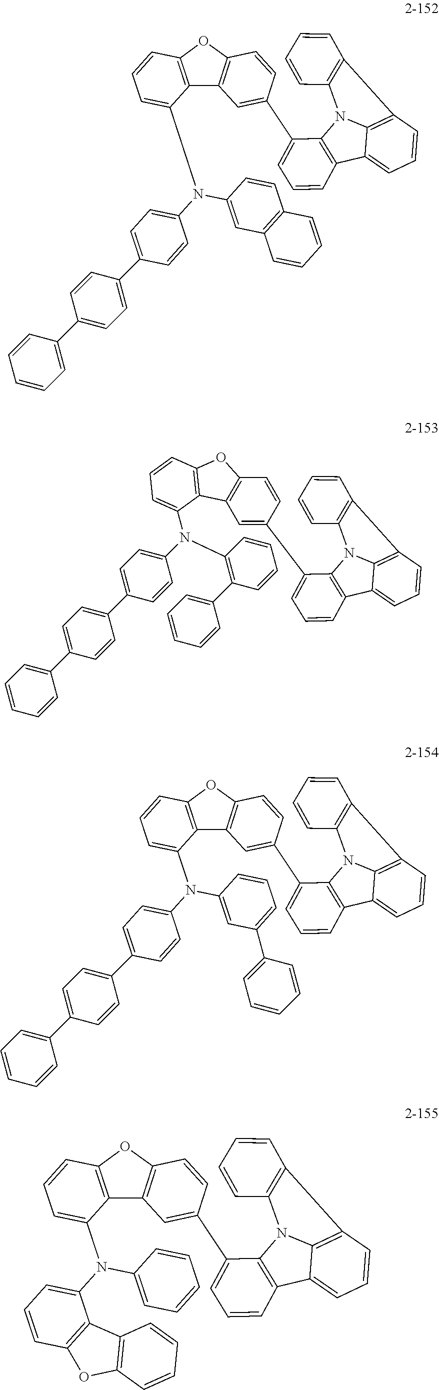 [SMILES: c1ccc(-c2ccc(-c3ccc(N(c4ccc5ccccc5c4)c4cccc5oc6ccc(-c7cccc8c9cccc%10c%11ccccc%11n(c78)c%109)cc6c45)cc3)cc2)cc1 ; c1ccc(-c2ccc(-c3ccc(N(c4ccccc4-c4ccccc4)c4cccc5oc6ccc(-c7cccc8c9cccc%10c%11ccccc%11n(c78)c%109)cc6c45)cc3)cc2)cc1 ; c1ccc(-c2ccc(-c3ccc(N(c4cccc(-c5ccccc5)c4)c4cccc5oc6ccc(-c7cccc8c9cccc%10c%11ccccc%11n(c78)c%109)cc6c45)cc3)cc2)cc1 ; c1ccc(N(c2cccc3oc4ccccc4c23)c2cccc3oc4ccc(-c5cccc6c7cccc8c9ccccc9n(c56)c87)cc4c23)cc1]

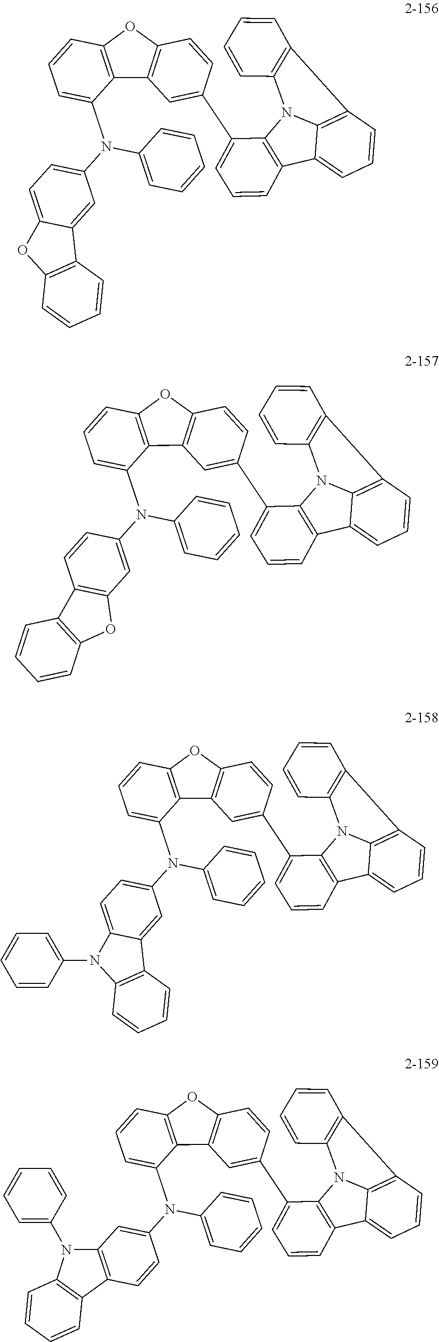 [SMILES: c1ccc(N(c2ccc3oc4ccccc4c3c2)c2cccc3oc4ccc(-c5cccc6c7cccc8c9ccccc9n(c56)c87)cc4c23)cc1 ; c1ccc(N(c2ccc3c(c2)oc2ccccc23)c2cccc3oc4ccc(-c5cccc6c7cccc8c9ccccc9n(c56)c87)cc4c23)cc1 ; c1ccc(N(c2ccc3c(c2)c2ccccc2n3-c2ccccc2)c2cccc3oc4ccc(-c5cccc6c7cccc8c9ccccc9n(c56)c87)cc4c23)cc1 ; c1ccc(N(c2ccc3c4ccccc4n(-c4ccccc4)c3c2)c2cccc3oc4ccc(-c5cccc6c7cccc8c9ccccc9n(c56)c87)cc4c23)cc1]

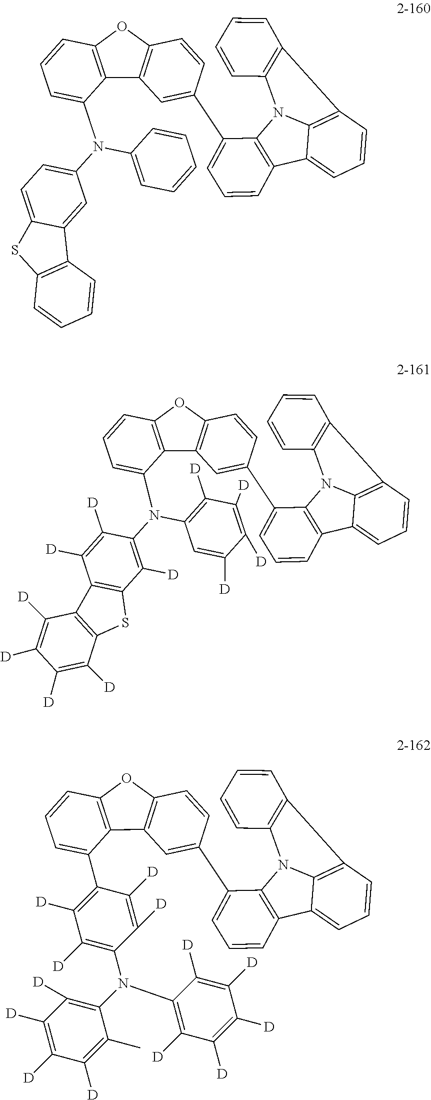 [SMILES: c1ccc(N(c2ccc3sc4ccccc4c3c2)c2cccc3oc4ccc(-c5cccc6c7cccc8c9ccccc9n(c56)c87)cc4c23)cc1 ; [2H]c1cc(N(c2c([2H])c([2H])c3c(sc4c([2H])c([2H])c([2H])c([2H])c43)c2[2H])c2cccc3oc4ccc(-c5cccc6c7cccc8c9ccccc9n(c56)c87)cc4c23)c([2H])c([2H])c1[2H] ; [2H]c1c([2H])c([2H])c(N(c2c([2H])c([2H])c(-c3cccc4oc5ccc(-c6cccc7c8cccc9c%10ccccc%10n(c67)c98)cc5c34)c([2H])c2[2H])c2c([2H])c([2H])c([2H])c([2H])c2C)c([2H])c1[2H]]

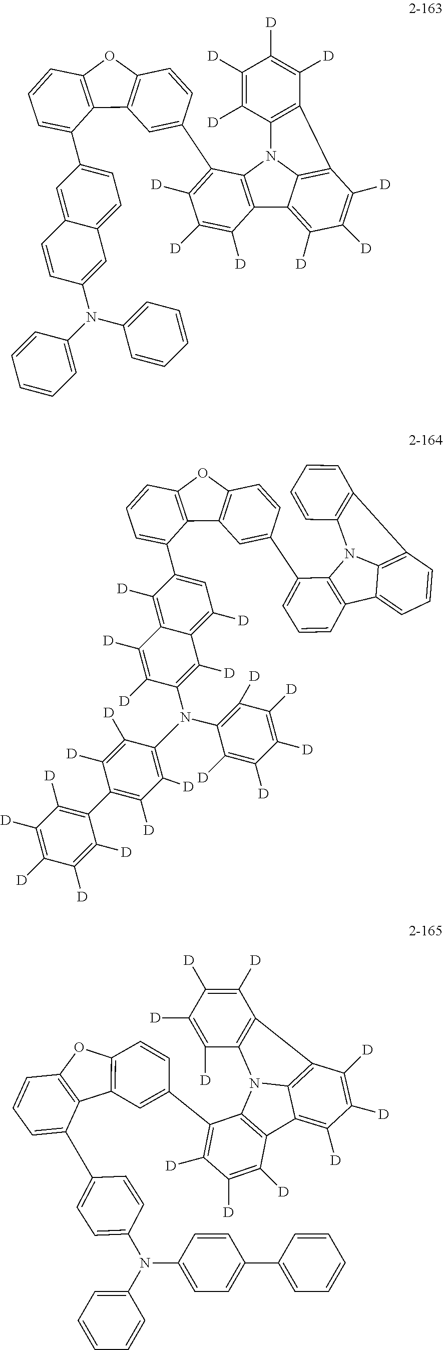

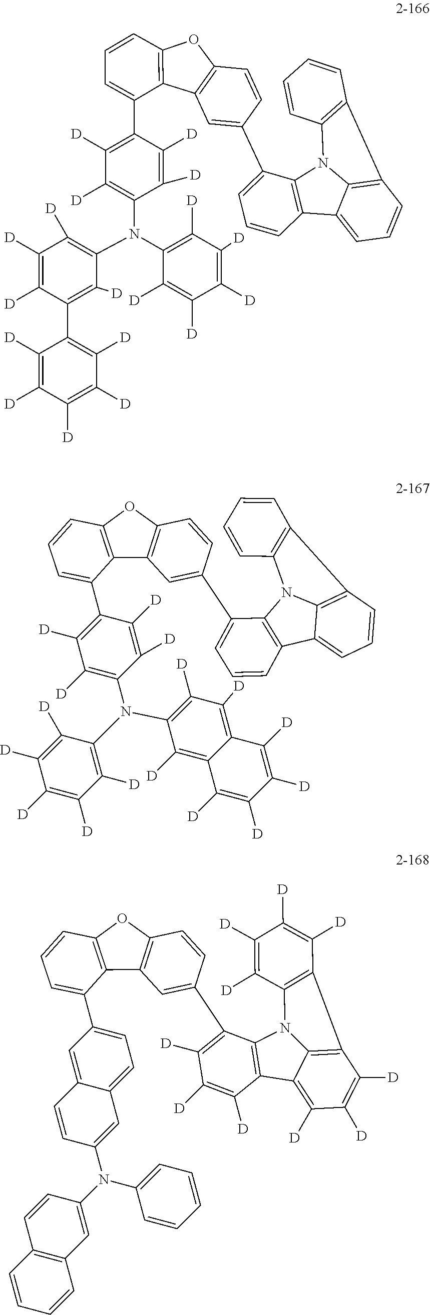

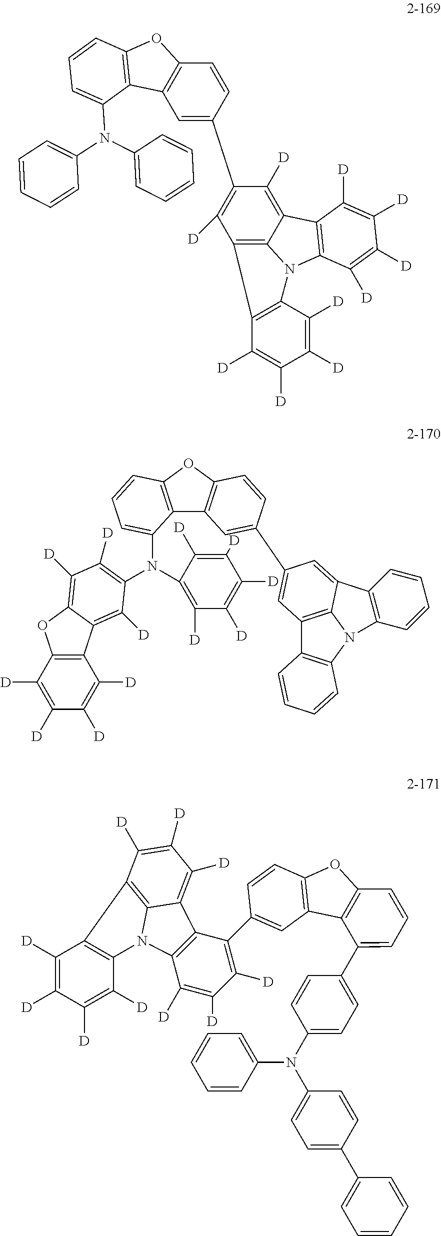 [SMILES: [2H]c1c([2H])c([2H])c2c(c1[2H])c1c([2H])c(-c3ccc4oc5cccc(N(c6ccccc6)c6ccccc6)c5c4c3)c([2H])c3c4c([2H])c([2H])c([2H])c([2H])c4n2c13 ; [2H]c1c([2H])c([2H])c(N(c2c([2H])c([2H])c3oc4c([2H])c([2H])c([2H])c([2H])c4c3c2[2H])c2cccc3oc4ccc(-c5cc6c7ccccc7n7c8ccccc8c(c5)c67)cc4c23)c([2H])c1[2H] ; [2H]c1c([2H])c([2H])c2c(c1[2H])c1c([2H])c([2H])c([2H])c3c4c(-c5ccc6oc7cccc(-c8ccc(N(c9ccccc9)c9ccc(-c%10ccccc%10)cc9)cc8)c7c6c5)c([2H])c([2H])c([2H])c4n2c13]

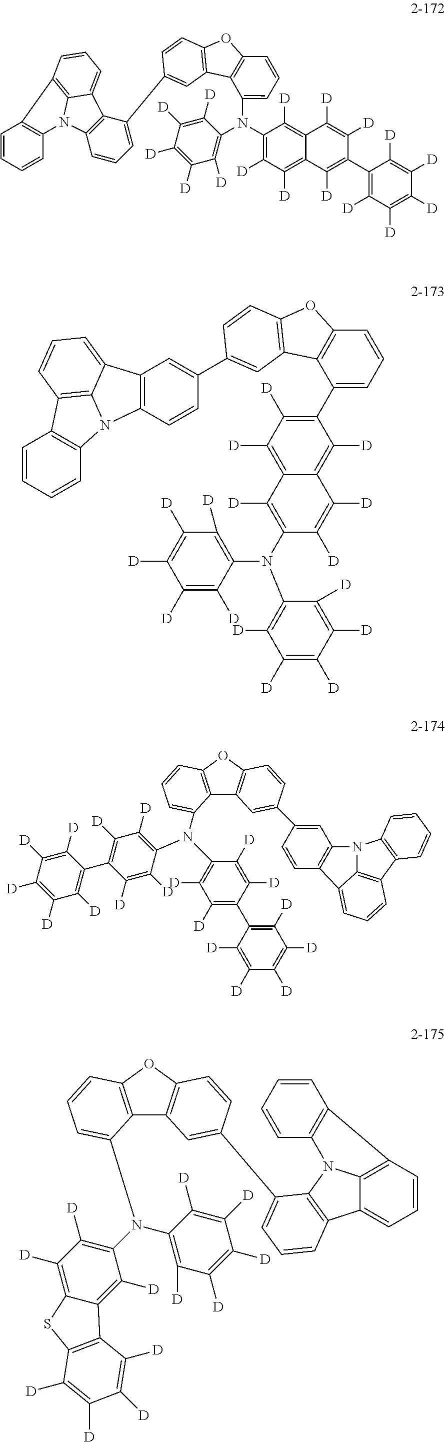

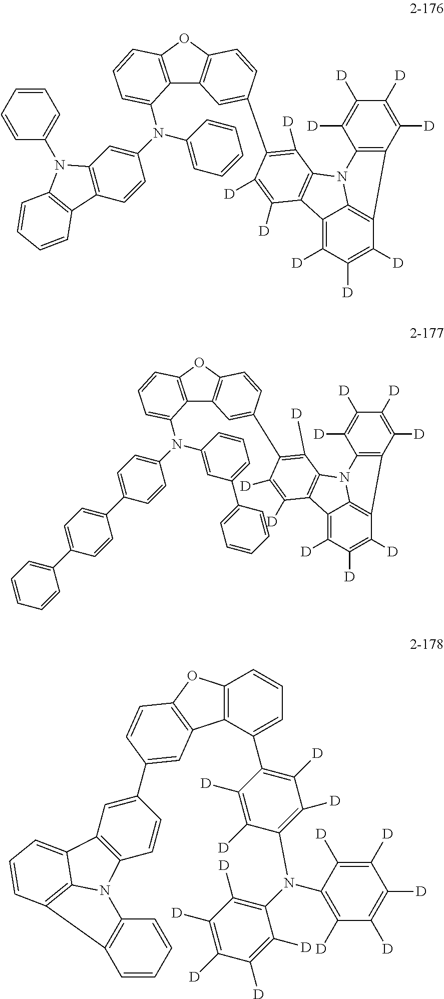 [SMILES: [2H]c1c([2H])c([2H])c2c(c1[2H])c1c([2H])c([2H])c([2H])c3c4c([2H])c([2H])c(-c5ccc6oc7cccc(N(c8ccccc8)c8ccc9c%10ccccc%10n(-c%10ccccc%10)c9c8)c7c6c5)c([2H])c4n2c13 ; [2H]c1c([2H])c([2H])c2c(c1[2H])c1c([2H])c([2H])c([2H])c3c4c([2H])c([2H])c(-c5ccc6oc7cccc(N(c8ccc(-c9ccc(-c%10ccccc%10)cc9)cc8)c8cccc(-c9ccccc9)c8)c7c6c5)c([2H])c4n2c13 ; [2H]c1c([2H])c([2H])c(N(c2c([2H])c([2H])c([2H])c([2H])c2[2H])c2c([2H])c([2H])c(-c3cccc4oc5ccc(-c6ccc7c(c6)c6cccc8c9ccccc9n7c86)cc5c34)c([2H])c2[2H])c([2H])c1[2H]]

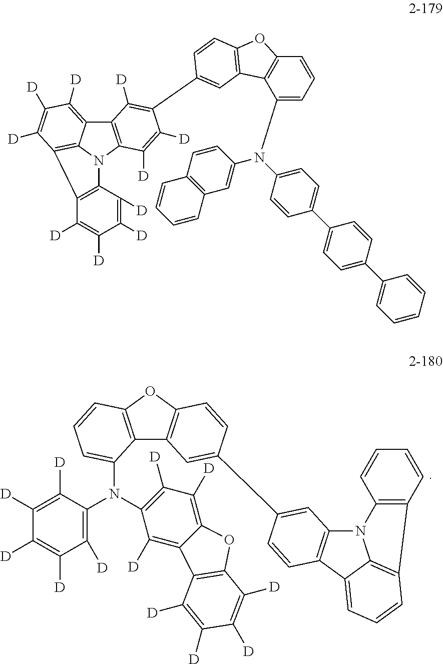 [SMILES: [2H]c1c([2H])c([2H])c2c(c1[2H])c1c([2H])c([2H])c([2H])c3c4c([2H])c(-c5ccc6oc7cccc(N(c8ccc(-c9ccc(-c%10ccccc%10)cc9)cc8)c8ccc9ccccc9c8)c7c6c5)c([2H])c([2H])c4n2c13 ; [2H]c1c([2H])c([2H])c(N(c2c([2H])c([2H])c3oc4c([2H])c([2H])c([2H])c([2H])c4c3c2[2H])c2cccc3oc4ccc(-c5ccc6c7cccc8c9ccccc9n(c6c5)c87)cc4c23)c([2H])c1[2H]]

7. An organic light emitting device comprising:
a first electrode;
a second electrode provided opposite to the first electrode; and
one or more organic material layers provided between the first electrode and the second electrode,
wherein one or more layers of the one or more organic material layers comprise the heterocyclic compound of claim 1.

8. The organic light emitting device of claim 7, wherein the organic material layer includes a light emitting layer, and the light emitting layer includes the heterocyclic compound.

9. The organic light emitting device of claim 7, wherein the organic material layer further includes a heterocyclic compound represented by the following Chemical Formula 2: 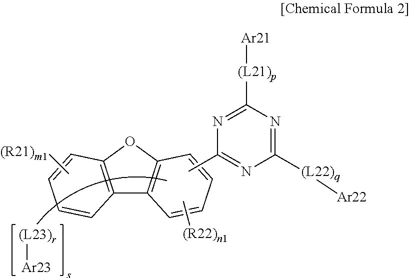
in Chemical Formula 2,
R21 and R22 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; —P(═O)R201R202; —SiR201R202R203; and —NR201R202, or two or more groups adjacent to each other bond to each other to form a substituted or unsubstituted C6 to C60 aromatic hydrocarbon ring; or a substituted or unsubstituted C2 to C60 heteroring, and R201, R202 and R203 are the same as or different from each other and each independently a substituted or unsubstituted C1 to C60 alkyl group; a substituted or unsubstituted C6 to C60 aryl group; or a substituted or unsubstituted C2 to C60 heteroaryl group;
m1 is an integer of 0 to 4, and when m1 is 2 or greater, R21s are the same as or different from each other;
n1 is an integer of 0 to 3, and when n1 is 2 or greater, R22s are the same as or different from each other;
L21 to L23 are the same as or different from each other, and each independently a direct bond; a substituted or unsubstituted C6 to C60 arylene group; or a substituted or unsubstituted C2 to C60 heteroarylene group;
p is an integer of 0 to 5, and when p is 2 or greater, L21s are the same as or different from each other;
q is an integer of 0 to 5, and when q is 2 or greater, L22s are the same as or different from each other;
r is an integer of 0 to 5, and when r is 2 or greater, L23s are the same as or different from each other;
Ar21 to Ar23 are the same as or different from each other, and each independently a substituted or unsubstituted C6 to C60 aryl group; or a substituted or unsubstituted C2 to C60 heteroaryl group; and
s is 0 or 1.

10. The organic light emitting device of claim 9, wherein the heterocyclic compound represented by Chemical Formula 2 is represented by any one of the following Chemical Formulae 2-1 to 2-3: 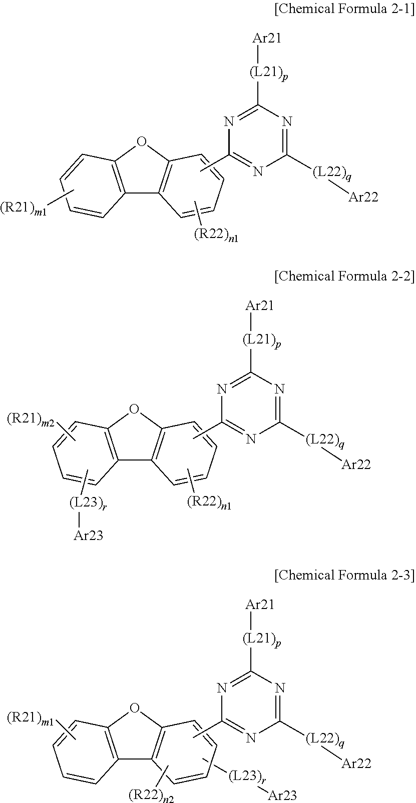
in Chemical Formulae 2-1 to 2-3,
m2 is an integer of 0 to 3, and when m2 is 2 or greater, R21s are the same as or different from each other;
n2 is an integer of 0 to 2, and when n2 is 2 or greater, R22s are the same as or different from each other; and
R21, R22, L21 to L23, Ar21 to Ar23, m1, n1 and p to r have the same definitions as in Chemical Formula 2.

11. The organic light emitting device of claim 9, wherein the heterocyclic compound represented by Chemical Formula 2 does not include deuterium as a substituent, or has a deuterium content of 1% to 100% with respect to a total number of hydrogen atoms and deuterium atoms.

12. The organic light emitting device of claim 9, wherein the heterocyclic compound represented by Chemical Formula 2 is represented by any one of the following compounds:

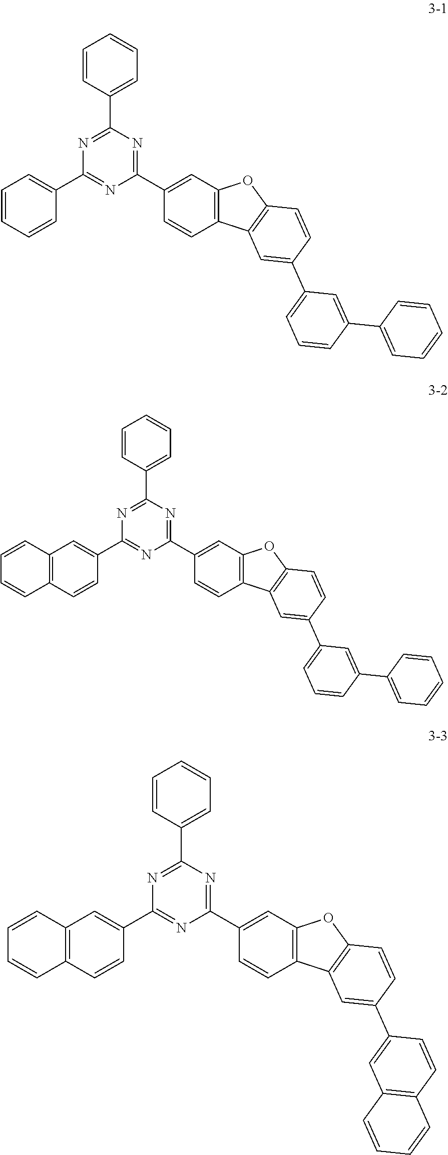 [SMILES: c1ccc(-c2cccc(-c3ccc4oc5cc(-c6nc(-c7ccccc7)nc(-c7ccccc7)n6)ccc5c4c3)c2)cc1 ; c1ccc(-c2cccc(-c3ccc4oc5cc(-c6nc(-c7ccccc7)nc(-c7ccc8ccccc8c7)n6)ccc5c4c3)c2)cc1 ; c1ccc(-c2nc(-c3ccc4ccccc4c3)nc(-c3ccc4c(c3)oc3ccc(-c5ccc6ccccc6c5)cc34)n2)cc1]

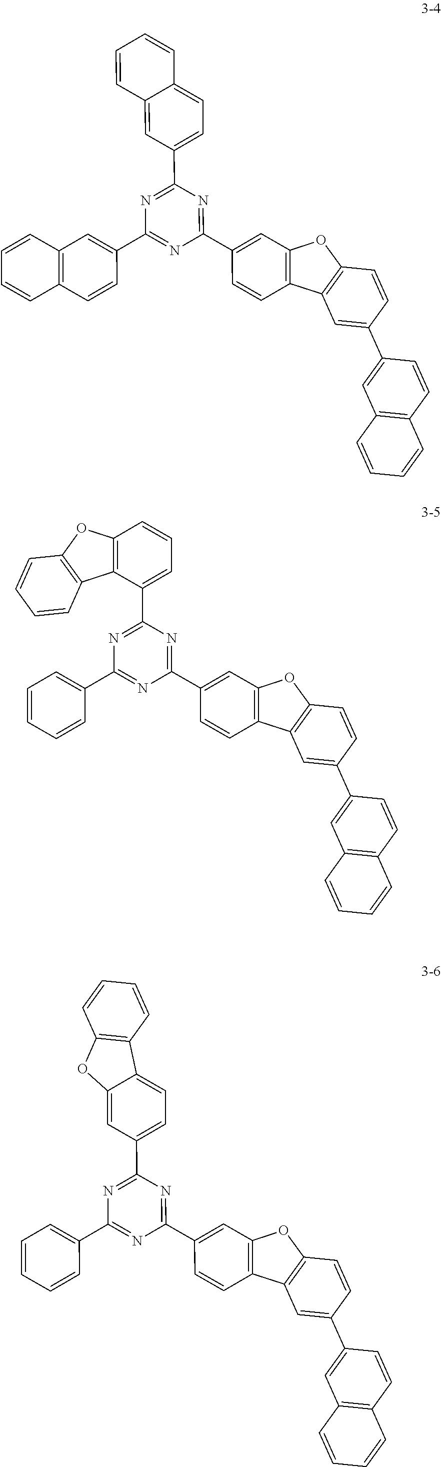 [SMILES: c1ccc2cc(-c3ccc4oc5cc(-c6nc(-c7ccc8ccccc8c7)nc(-c7ccc8ccccc8c7)n6)ccc5c4c3)ccc2c1 ; c1ccc(-c2nc(-c3ccc4c(c3)oc3ccc(-c5ccc6ccccc6c5)cc34)nc(-c3cccc4oc5ccccc5c34)n2)cc1 ; c1ccc(-c2nc(-c3ccc4c(c3)oc3ccccc34)nc(-c3ccc4c(c3)oc3ccc(-c5ccc6ccccc6c5)cc34)n2)cc1]

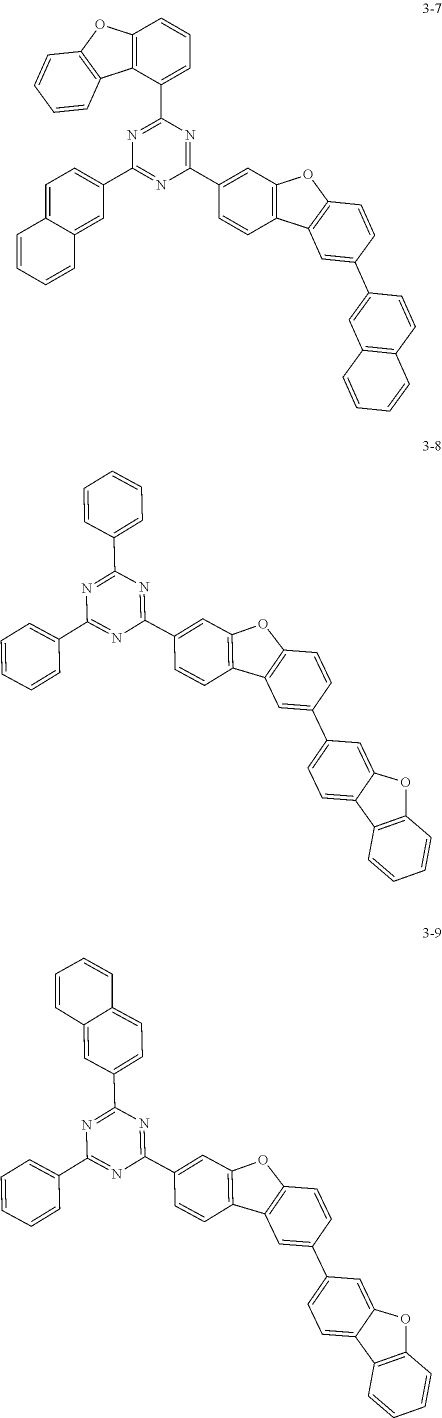 [SMILES: c1ccc2cc(-c3ccc4oc5cc(-c6nc(-c7ccc8ccccc8c7)nc(-c7cccc8oc9ccccc9c78)n6)ccc5c4c3)ccc2c1 ; c1ccc(-c2nc(-c3ccccc3)nc(-c3ccc4c(c3)oc3ccc(-c5ccc6c(c5)oc5ccccc56)cc34)n2)cc1 ; c1ccc(-c2nc(-c3ccc4ccccc4c3)nc(-c3ccc4c(c3)oc3ccc(-c5ccc6c(c5)oc5ccccc56)cc34)n2)cc1]

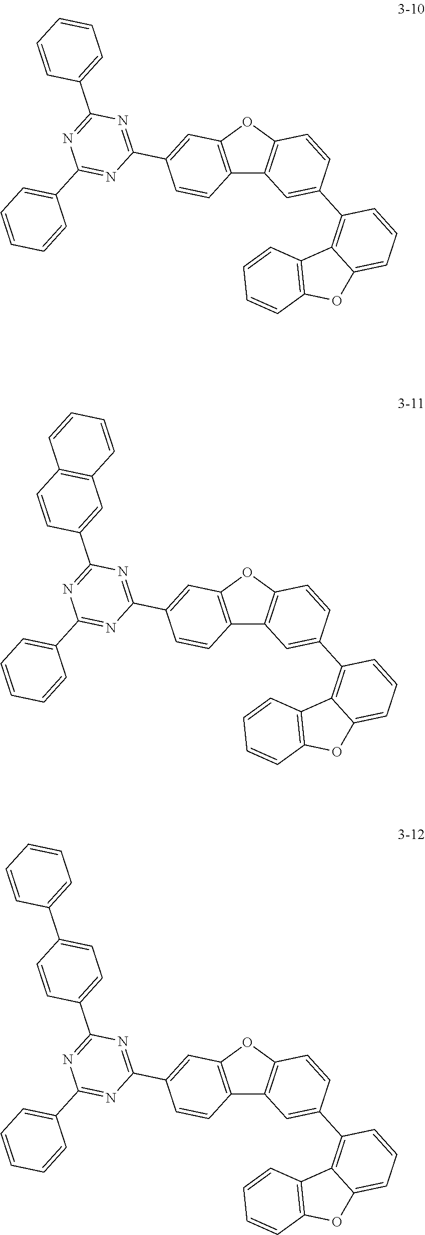 [SMILES: c1ccc(-c2nc(-c3ccccc3)nc(-c3ccc4c(c3)oc3ccc(-c5cccc6oc7ccccc7c56)cc34)n2)cc1 ; c1ccc(-c2nc(-c3ccc4ccccc4c3)nc(-c3ccc4c(c3)oc3ccc(-c5cccc6oc7ccccc7c56)cc34)n2)cc1 ; c1ccc(-c2ccc(-c3nc(-c4ccccc4)nc(-c4ccc5c(c4)oc4ccc(-c6cccc7oc8ccccc8c67)cc45)n3)cc2)cc1]

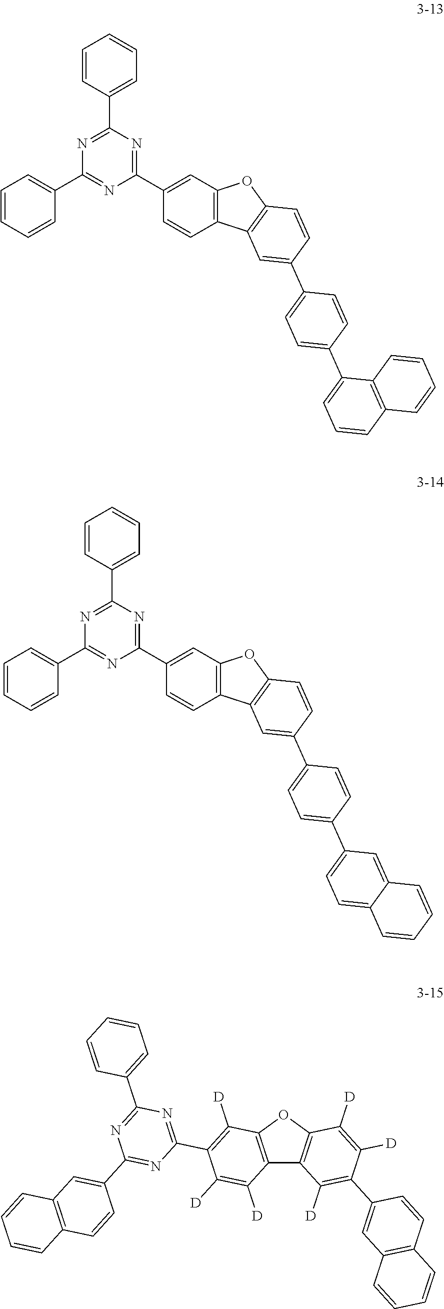 [SMILES: c1ccc(-c2nc(-c3ccccc3)nc(-c3ccc4c(c3)oc3ccc(-c5ccc(-c6cccc7ccccc67)cc5)cc34)n2)cc1 ; c1ccc(-c2nc(-c3ccccc3)nc(-c3ccc4c(c3)oc3ccc(-c5ccc(-c6ccc7ccccc7c6)cc5)cc34)n2)cc1 ; [2H]c1c(-c2ccc3ccccc3c2)c([2H])c2c(oc3c([2H])c(-c4nc(-c5ccccc5)nc(-c5ccc6ccccc6c5)n4)c([2H])c([2H])c32)c1[2H]]

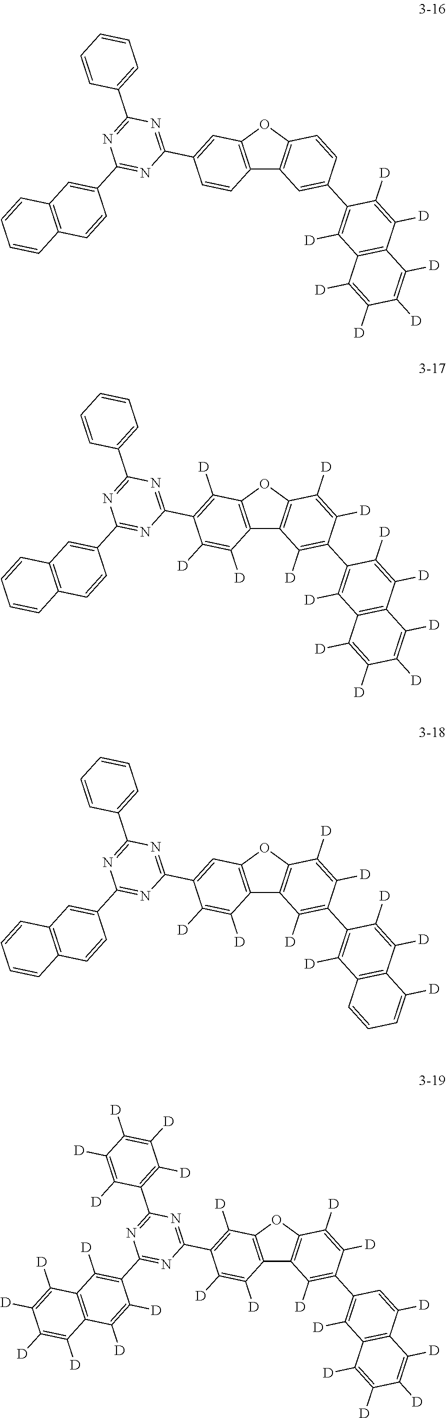

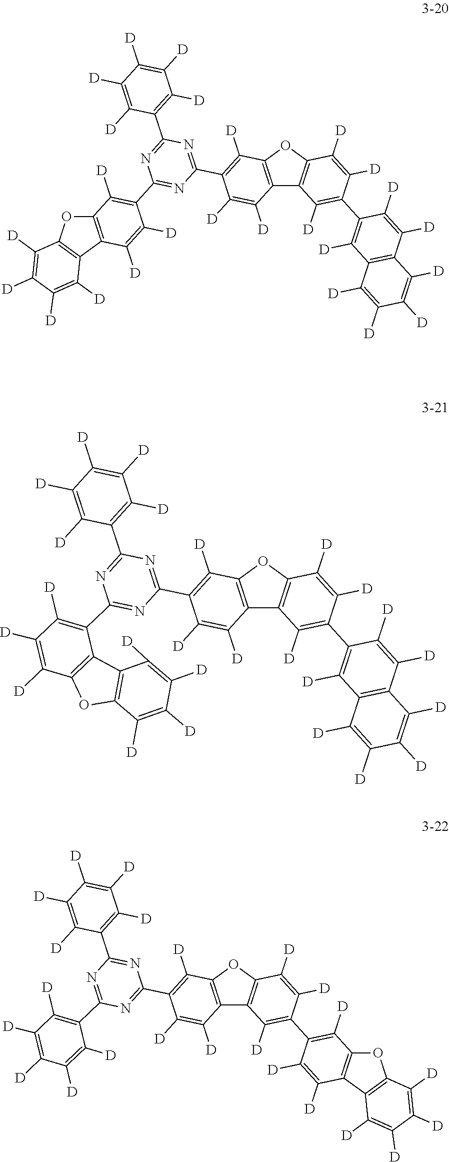

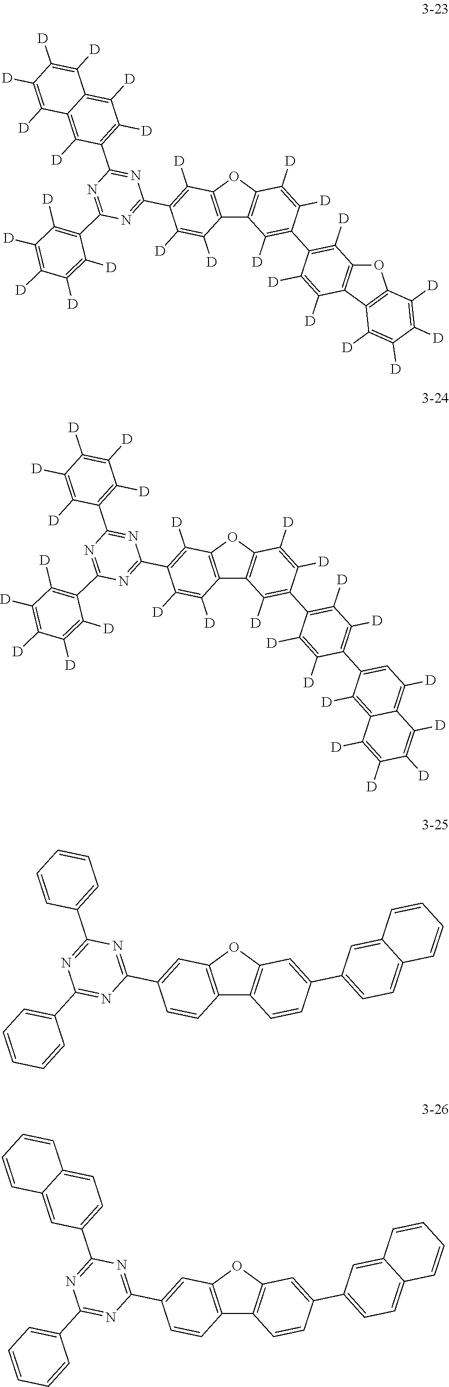

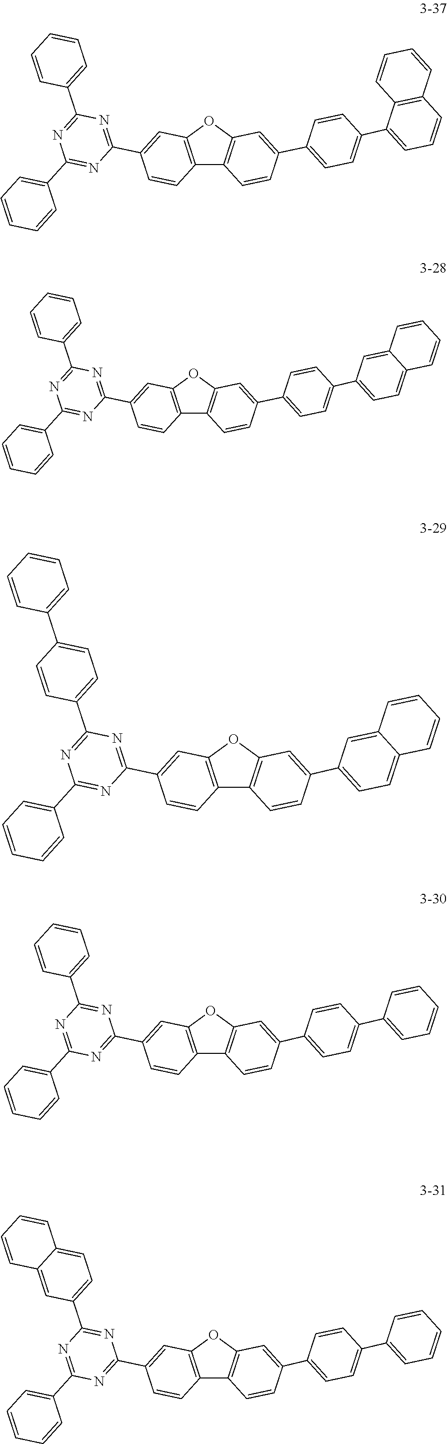 [SMILES: c1ccc(-c2nc(-c3ccccc3)nc(-c3ccc4c(c3)oc3cc(-c5ccc(-c6cccc7ccccc67)cc5)ccc34)n2)cc1 ; c1ccc(-c2nc(-c3ccccc3)nc(-c3ccc4c(c3)oc3cc(-c5ccc(-c6ccc7ccccc7c6)cc5)ccc34)n2)cc1 ; c1ccc(-c2ccc(-c3nc(-c4ccccc4)nc(-c4ccc5c(c4)oc4cc(-c6ccc7ccccc7c6)ccc45)n3)cc2)cc1 ; c1ccc(-c2ccc(-c3ccc4c(c3)oc3cc(-c5nc(-c6ccccc6)nc(-c6ccccc6)n5)ccc34)cc2)cc1 ; c1ccc(-c2ccc(-c3ccc4c(c3)oc3cc(-c5nc(-c6ccccc6)nc(-c6ccc7ccccc7c6)n5)ccc34)cc2)cc1]

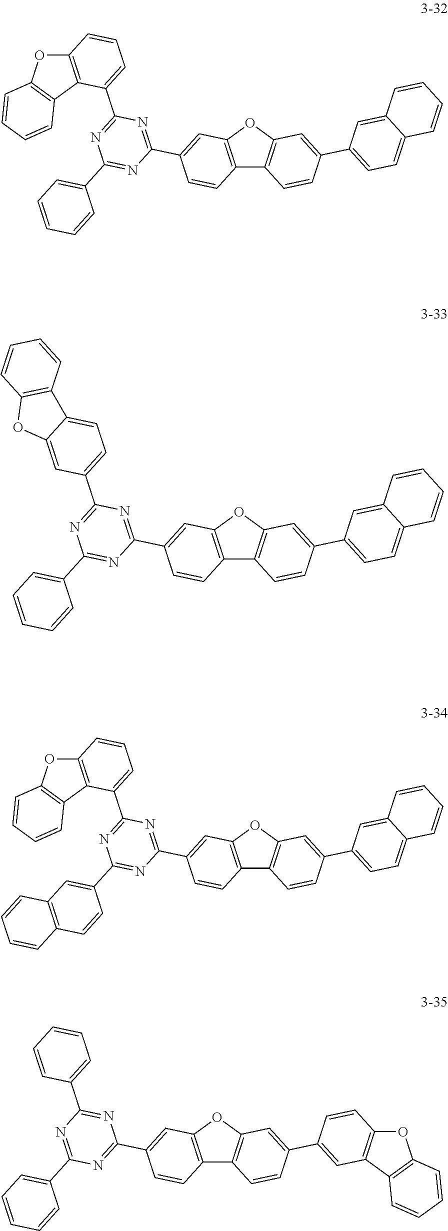 [SMILES: c1ccc(-c2nc(-c3ccc4c(c3)oc3cc(-c5ccc6ccccc6c5)ccc34)nc(-c3cccc4oc5ccccc5c34)n2)cc1 ; c1ccc(-c2nc(-c3ccc4c(c3)oc3ccccc34)nc(-c3ccc4c(c3)oc3cc(-c5ccc6ccccc6c5)ccc34)n2)cc1 ; c1ccc2cc(-c3ccc4c(c3)oc3cc(-c5nc(-c6ccc7ccccc7c6)nc(-c6cccc7oc8ccccc8c67)n5)ccc34)ccc2c1 ; c1ccc(-c2nc(-c3ccccc3)nc(-c3ccc4c(c3)oc3cc(-c5ccc6oc7ccccc7c6c5)ccc34)n2)cc1]

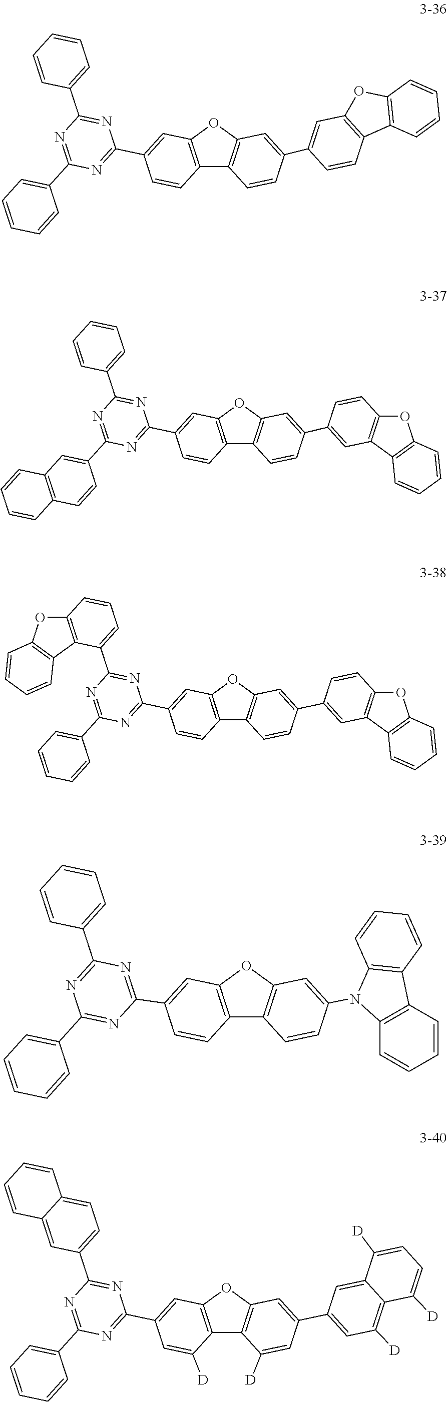 [SMILES: c1ccc(-c2nc(-c3ccccc3)nc(-c3ccc4c(c3)oc3cc(-c5ccc6c(c5)oc5ccccc56)ccc34)n2)cc1 ; c1ccc(-c2nc(-c3ccc4ccccc4c3)nc(-c3ccc4c(c3)oc3cc(-c5ccc6oc7ccccc7c6c5)ccc34)n2)cc1 ; c1ccc(-c2nc(-c3ccc4c(c3)oc3cc(-c5ccc6oc7ccccc7c6c5)ccc34)nc(-c3cccc4oc5ccccc5c34)n2)cc1 ; c1ccc(-c2nc(-c3ccccc3)nc(-c3ccc4c(c3)oc3cc(-n5c6ccccc6c6ccccc65)ccc34)n2)cc1 ; [2H]c1ccc([2H])c2c([2H])cc(-c3cc([2H])c4c(c3)oc3cc(-c5nc(-c6ccccc6)nc(-c6ccc7ccccc7c6)n5)cc([2H])c34)cc12]

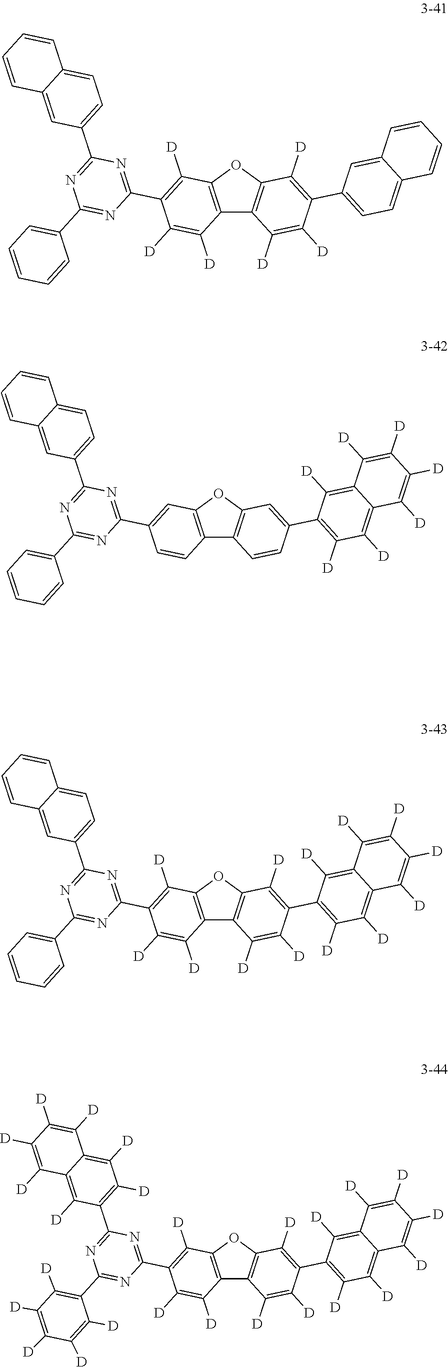

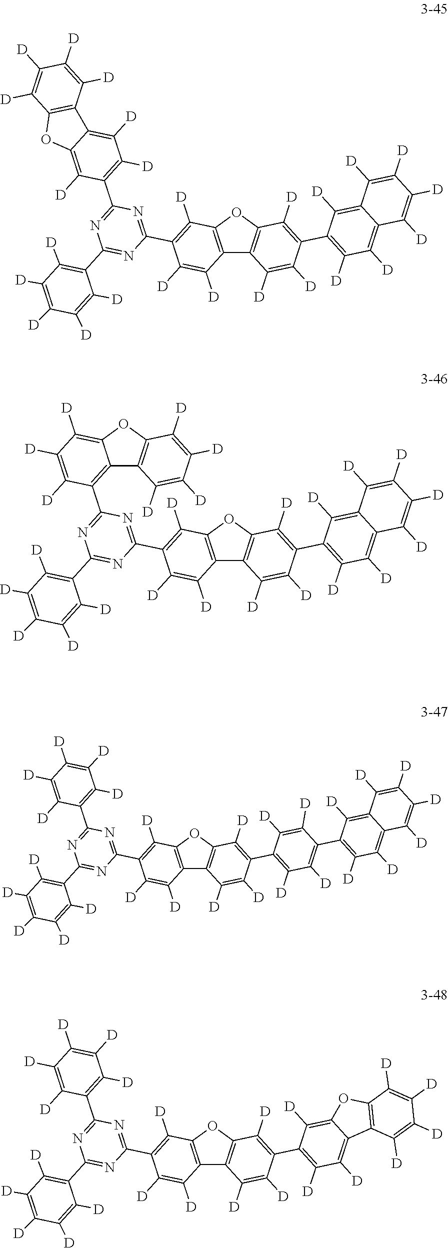

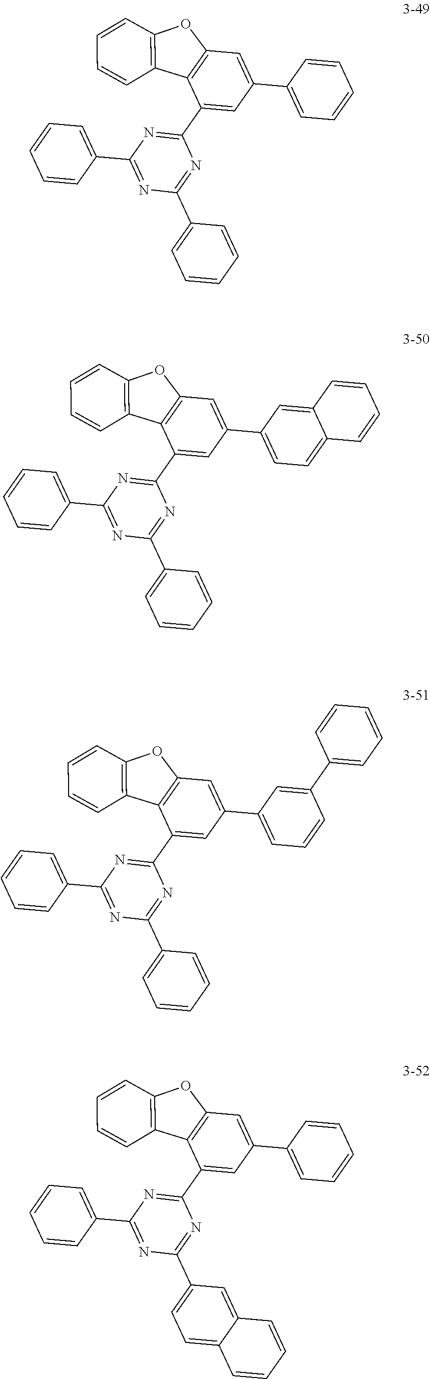 [SMILES: c1ccc(-c2cc(-c3nc(-c4ccccc4)nc(-c4ccccc4)n3)c3c(c2)oc2ccccc23)cc1 ; c1ccc(-c2nc(-c3ccccc3)nc(-c3cc(-c4ccc5ccccc5c4)cc4oc5ccccc5c34)n2)cc1 ; c1ccc(-c2cccc(-c3cc(-c4nc(-c5ccccc5)nc(-c5ccccc5)n4)c4c(c3)oc3ccccc34)c2)cc1 ; c1ccc(-c2cc(-c3nc(-c4ccccc4)nc(-c4ccc5ccccc5c4)n3)c3c(c2)oc2ccccc23)cc1]

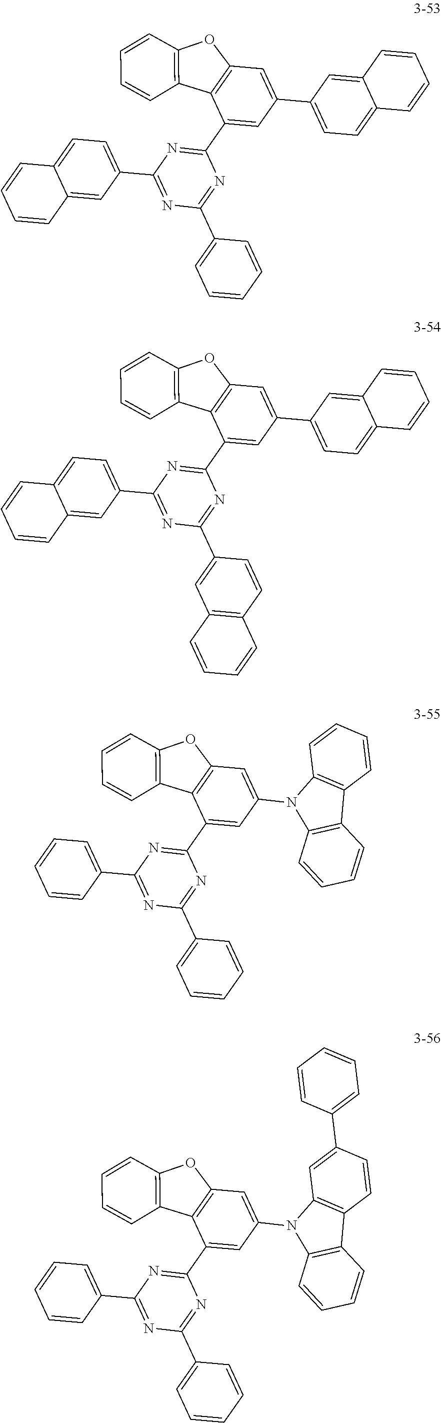 [SMILES: c1ccc(-c2nc(-c3ccc4ccccc4c3)nc(-c3cc(-c4ccc5ccccc5c4)cc4oc5ccccc5c34)n2)cc1 ; c1ccc2cc(-c3cc(-c4nc(-c5ccc6ccccc6c5)nc(-c5ccc6ccccc6c5)n4)c4c(c3)oc3ccccc34)ccc2c1 ; c1ccc(-c2nc(-c3ccccc3)nc(-c3cc(-n4c5ccccc5c5ccccc54)cc4oc5ccccc5c34)n2)cc1 ; c1ccc(-c2ccc3c4ccccc4n(-c4cc(-c5nc(-c6ccccc6)nc(-c6ccccc6)n5)c5c(c4)oc4ccccc45)c3c2)cc1]

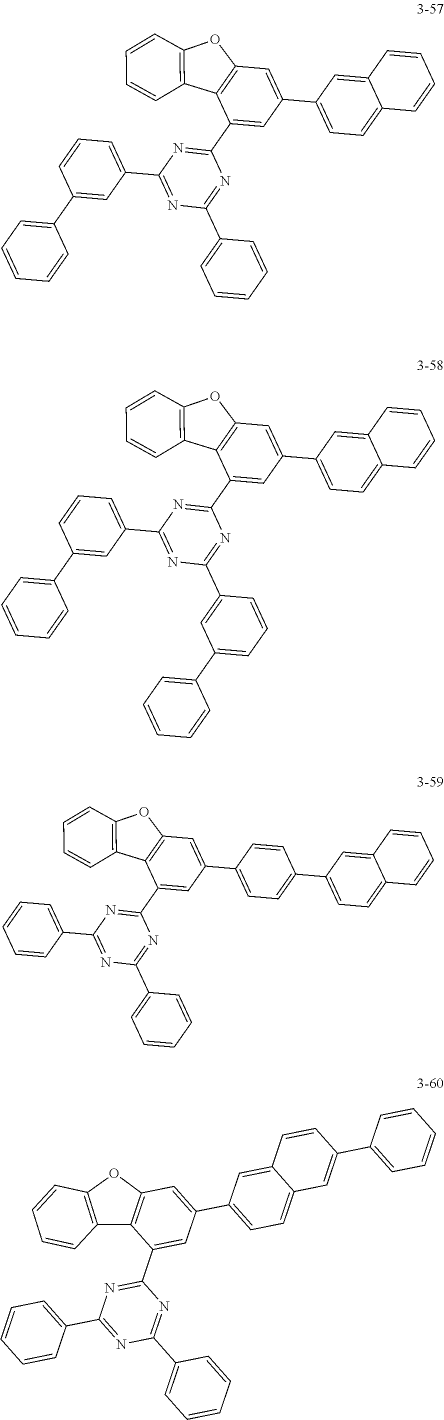 [SMILES: c1ccc(-c2cccc(-c3nc(-c4ccccc4)nc(-c4cc(-c5ccc6ccccc6c5)cc5oc6ccccc6c45)n3)c2)cc1 ; c1ccc(-c2cccc(-c3nc(-c4cccc(-c5ccccc5)c4)nc(-c4cc(-c5ccc6ccccc6c5)cc5oc6ccccc6c45)n3)c2)cc1 ; c1ccc(-c2nc(-c3ccccc3)nc(-c3cc(-c4ccc(-c5ccc6ccccc6c5)cc4)cc4oc5ccccc5c34)n2)cc1 ; c1ccc(-c2ccc3cc(-c4cc(-c5nc(-c6ccccc6)nc(-c6ccccc6)n5)c5c(c4)oc4ccccc45)ccc3c2)cc1]

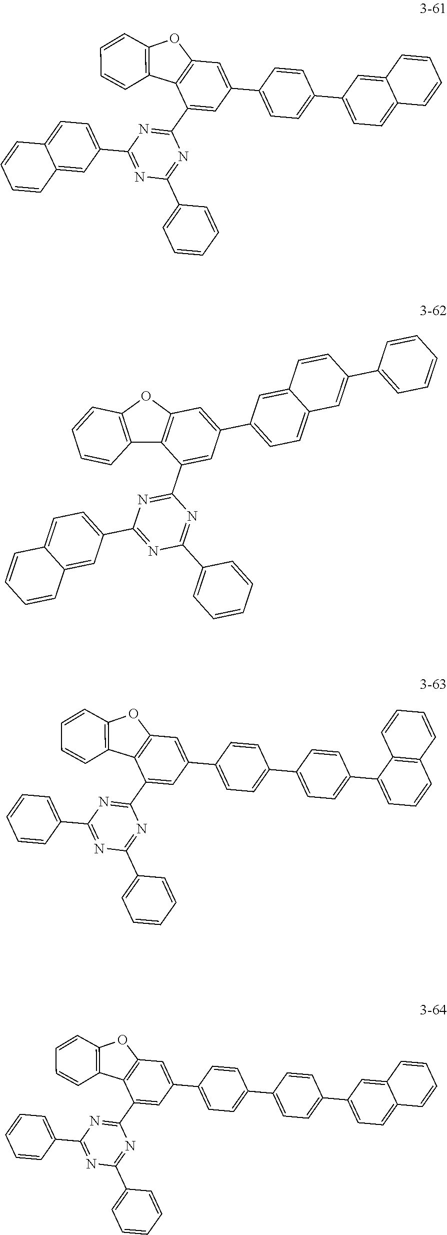 [SMILES: c1ccc(-c2nc(-c3ccc4ccccc4c3)nc(-c3cc(-c4ccc(-c5ccc6ccccc6c5)cc4)cc4oc5ccccc5c34)n2)cc1 ; c1ccc(-c2ccc3cc(-c4cc(-c5nc(-c6ccccc6)nc(-c6ccc7ccccc7c6)n5)c5c(c4)oc4ccccc45)ccc3c2)cc1 ; c1ccc(-c2nc(-c3ccccc3)nc(-c3cc(-c4ccc(-c5ccc(-c6cccc7ccccc67)cc5)cc4)cc4oc5ccccc5c34)n2)cc1 ; c1ccc(-c2nc(-c3ccccc3)nc(-c3cc(-c4ccc(-c5ccc(-c6ccc7ccccc7c6)cc5)cc4)cc4oc5ccccc5c34)n2)cc1]

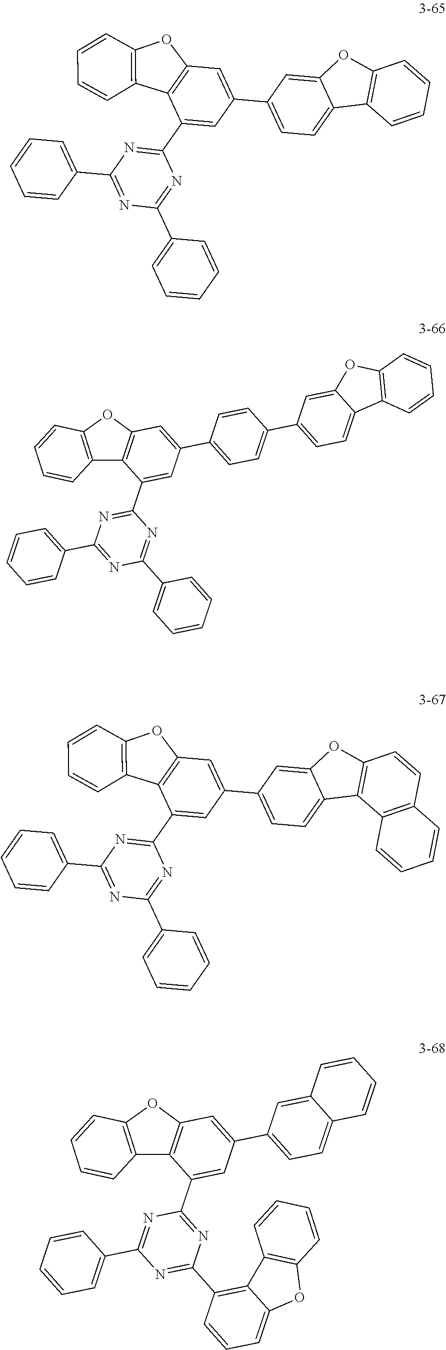 [SMILES: c1ccc(-c2nc(-c3ccccc3)nc(-c3cc(-c4ccc5c(c4)oc4ccccc45)cc4oc5ccccc5c34)n2)cc1 ; c1ccc(-c2nc(-c3ccccc3)nc(-c3cc(-c4ccc(-c5ccc6c(c5)oc5ccccc56)cc4)cc4oc5ccccc5c34)n2)cc1 ; c1ccc(-c2nc(-c3ccccc3)nc(-c3cc(-c4ccc5c(c4)oc4ccc6ccccc6c45)cc4oc5ccccc5c34)n2)cc1 ; c1ccc(-c2nc(-c3cccc4oc5ccccc5c34)nc(-c3cc(-c4ccc5ccccc5c4)cc4oc5ccccc5c34)n2)cc1]

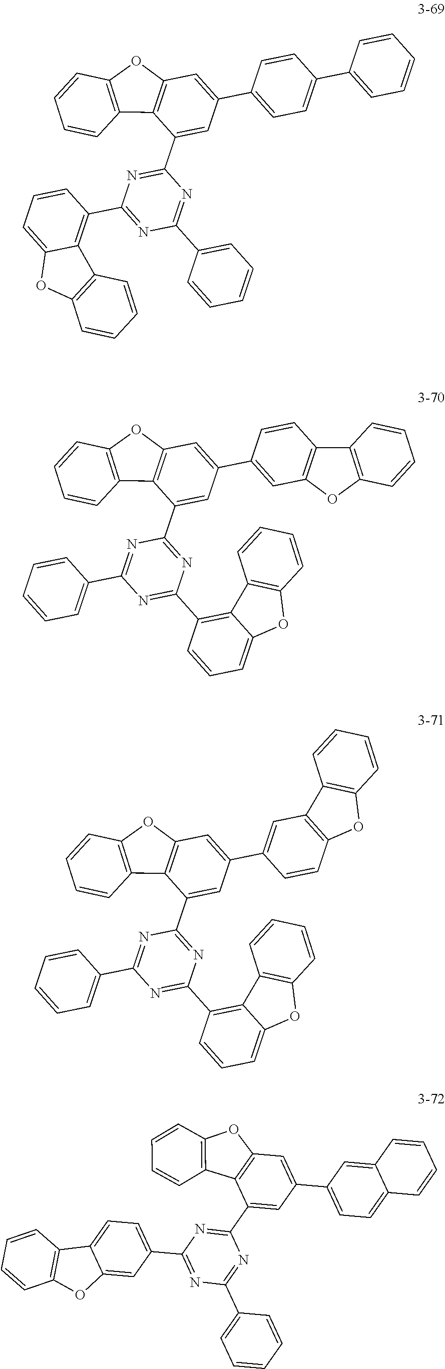 [SMILES: c1ccc(-c2ccc(-c3cc(-c4nc(-c5ccccc5)nc(-c5cccc6oc7ccccc7c56)n4)c4c(c3)oc3ccccc34)cc2)cc1 ; c1ccc(-c2nc(-c3cccc4oc5ccccc5c34)nc(-c3cc(-c4ccc5c(c4)oc4ccccc45)cc4oc5ccccc5c34)n2)cc1 ; c1ccc(-c2nc(-c3cccc4oc5ccccc5c34)nc(-c3cc(-c4ccc5oc6ccccc6c5c4)cc4oc5ccccc5c34)n2)cc1 ; c1ccc(-c2nc(-c3ccc4c(c3)oc3ccccc34)nc(-c3cc(-c4ccc5ccccc5c4)cc4oc5ccccc5c34)n2)cc1]

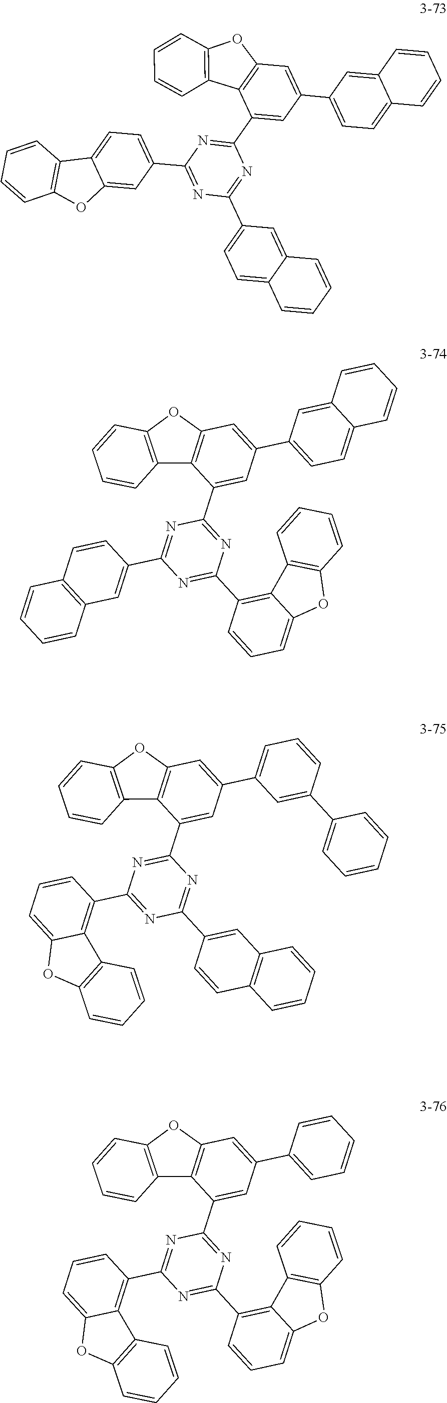 [SMILES: c1ccc2cc(-c3cc(-c4nc(-c5ccc6ccccc6c5)nc(-c5ccc6c(c5)oc5ccccc56)n4)c4c(c3)oc3ccccc34)ccc2c1 ; c1ccc2cc(-c3cc(-c4nc(-c5ccc6ccccc6c5)nc(-c5cccc6oc7ccccc7c56)n4)c4c(c3)oc3ccccc34)ccc2c1 ; c1ccc(-c2cccc(-c3cc(-c4nc(-c5ccc6ccccc6c5)nc(-c5cccc6oc7ccccc7c56)n4)c4c(c3)oc3ccccc34)c2)cc1 ; c1ccc(-c2cc(-c3nc(-c4cccc5oc6ccccc6c45)nc(-c4cccc5oc6ccccc6c45)n3)c3c(c2)oc2ccccc23)cc1]

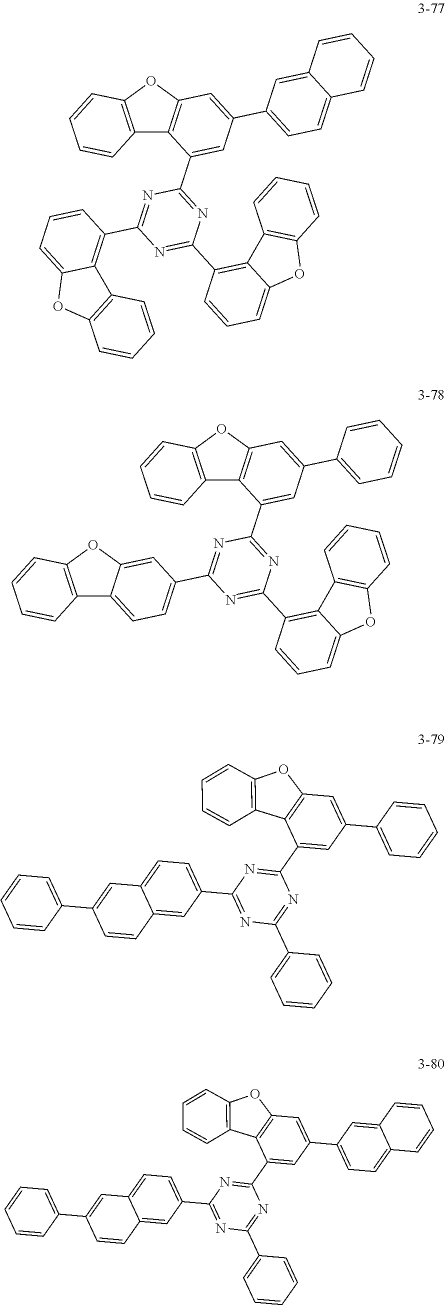 [SMILES: c1ccc2cc(-c3cc(-c4nc(-c5cccc6oc7ccccc7c56)nc(-c5cccc6oc7ccccc7c56)n4)c4c(c3)oc3ccccc34)ccc2c1 ; c1ccc(-c2cc(-c3nc(-c4ccc5c(c4)oc4ccccc45)nc(-c4cccc5oc6ccccc6c45)n3)c3c(c2)oc2ccccc23)cc1 ; c1ccc(-c2ccc3cc(-c4nc(-c5ccccc5)nc(-c5cc(-c6ccccc6)cc6oc7ccccc7c56)n4)ccc3c2)cc1 ; c1ccc(-c2ccc3cc(-c4nc(-c5ccccc5)nc(-c5cc(-c6ccc7ccccc7c6)cc6oc7ccccc7c56)n4)ccc3c2)cc1]

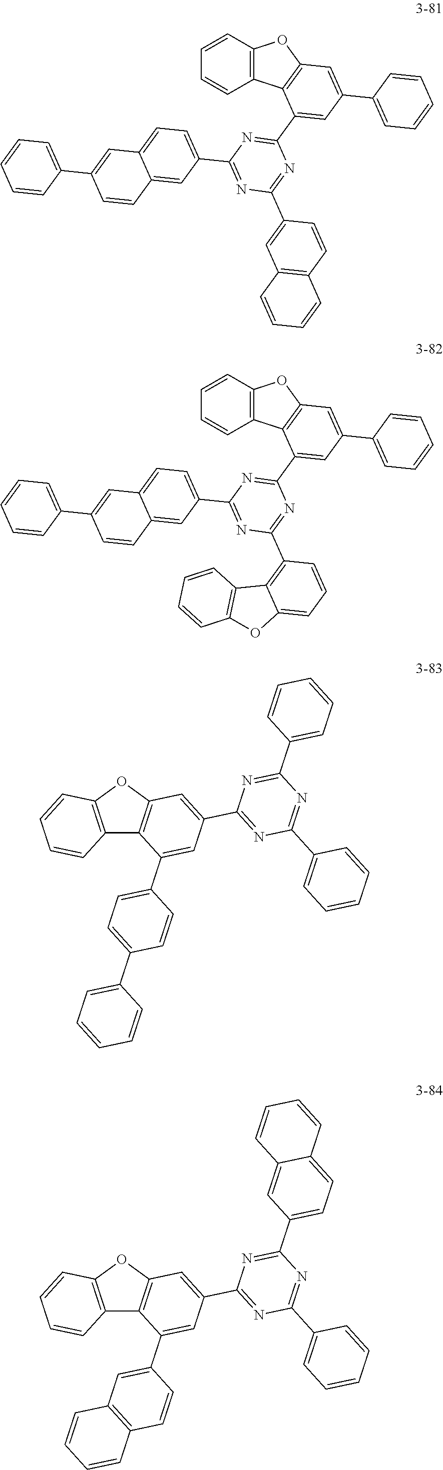 [SMILES: c1ccc(-c2ccc3cc(-c4nc(-c5ccc6ccccc6c5)nc(-c5cc(-c6ccccc6)cc6oc7ccccc7c56)n4)ccc3c2)cc1 ; c1ccc(-c2ccc3cc(-c4nc(-c5cccc6oc7ccccc7c56)nc(-c5cc(-c6ccccc6)cc6oc7ccccc7c56)n4)ccc3c2)cc1 ; c1ccc(-c2ccc(-c3cc(-c4nc(-c5ccccc5)nc(-c5ccccc5)n4)cc4oc5ccccc5c34)cc2)cc1 ; c1ccc(-c2nc(-c3ccc4ccccc4c3)nc(-c3cc(-c4ccc5ccccc5c4)c4c(c3)oc3ccccc34)n2)cc1]

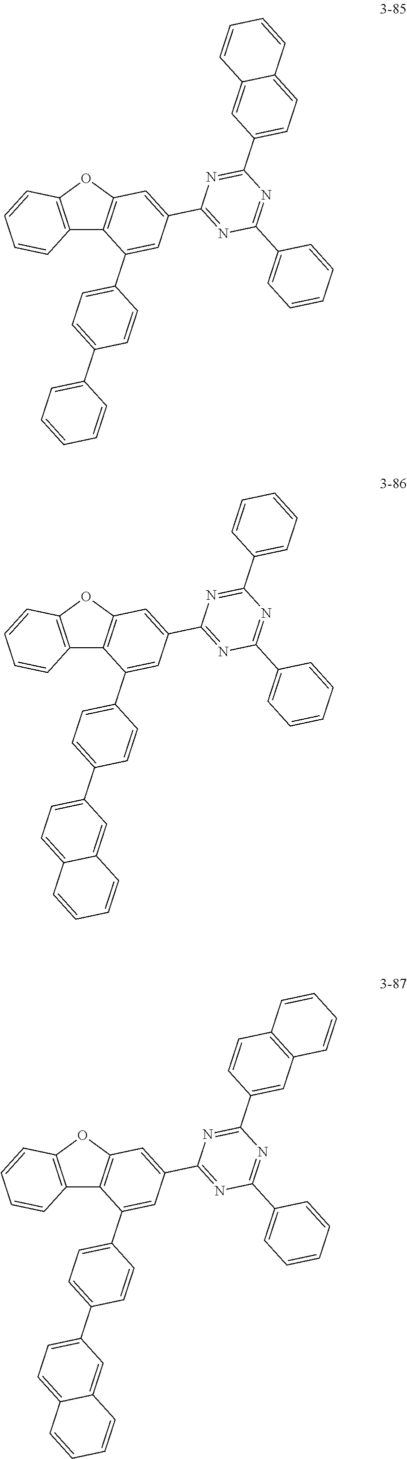 [SMILES: c1ccc(-c2ccc(-c3cc(-c4nc(-c5ccccc5)nc(-c5ccc6ccccc6c5)n4)cc4oc5ccccc5c34)cc2)cc1 ; c1ccc(-c2nc(-c3ccccc3)nc(-c3cc(-c4ccc(-c5ccc6ccccc6c5)cc4)c4c(c3)oc3ccccc34)n2)cc1 ; c1ccc(-c2nc(-c3ccc4ccccc4c3)nc(-c3cc(-c4ccc(-c5ccc6ccccc6c5)cc4)c4c(c3)oc3ccccc34)n2)cc1]

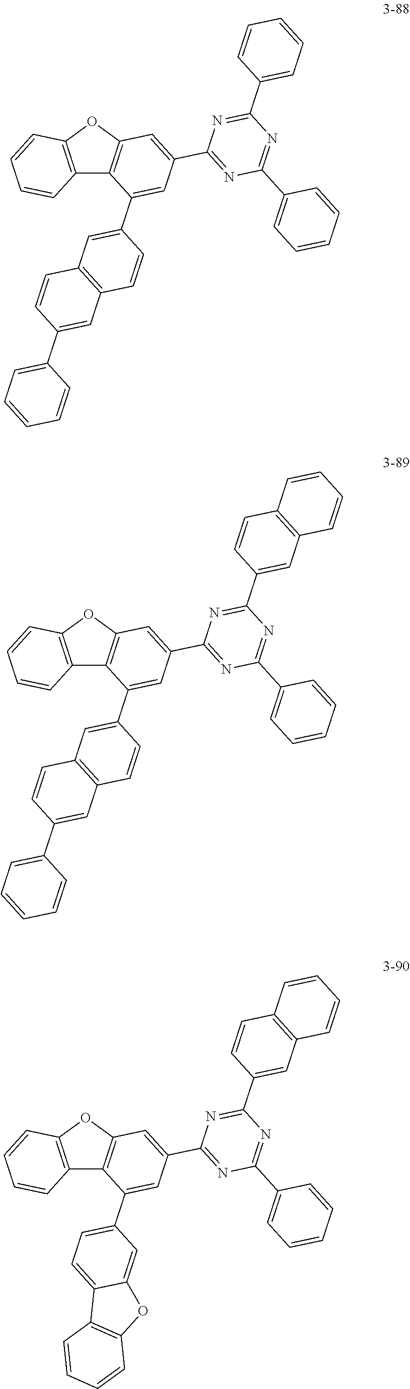 [SMILES: c1ccc(-c2ccc3cc(-c4cc(-c5nc(-c6ccccc6)nc(-c6ccccc6)n5)cc5oc6ccccc6c45)ccc3c2)cc1 ; c1ccc(-c2ccc3cc(-c4cc(-c5nc(-c6ccccc6)nc(-c6ccc7ccccc7c6)n5)cc5oc6ccccc6c45)ccc3c2)cc1 ; c1ccc(-c2nc(-c3ccc4ccccc4c3)nc(-c3cc(-c4ccc5c(c4)oc4ccccc45)c4c(c3)oc3ccccc34)n2)cc1]

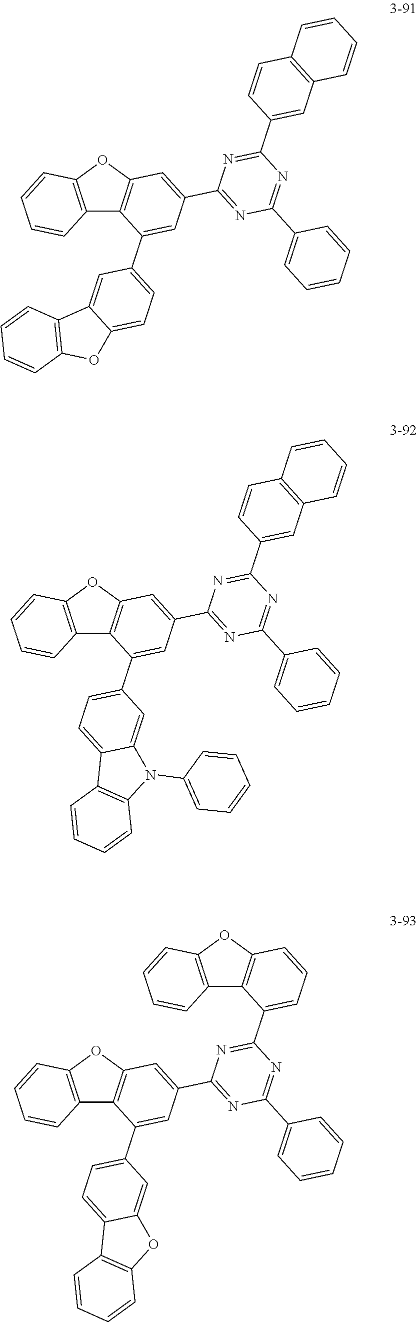 [SMILES: c1ccc(-c2nc(-c3ccc4ccccc4c3)nc(-c3cc(-c4ccc5oc6ccccc6c5c4)c4c(c3)oc3ccccc34)n2)cc1 ; c1ccc(-c2nc(-c3ccc4ccccc4c3)nc(-c3cc(-c4ccc5c6ccccc6n(-c6ccccc6)c5c4)c4c(c3)oc3ccccc34)n2)cc1 ; c1ccc(-c2nc(-c3cc(-c4ccc5c(c4)oc4ccccc45)c4c(c3)oc3ccccc34)nc(-c3cccc4oc5ccccc5c34)n2)cc1]

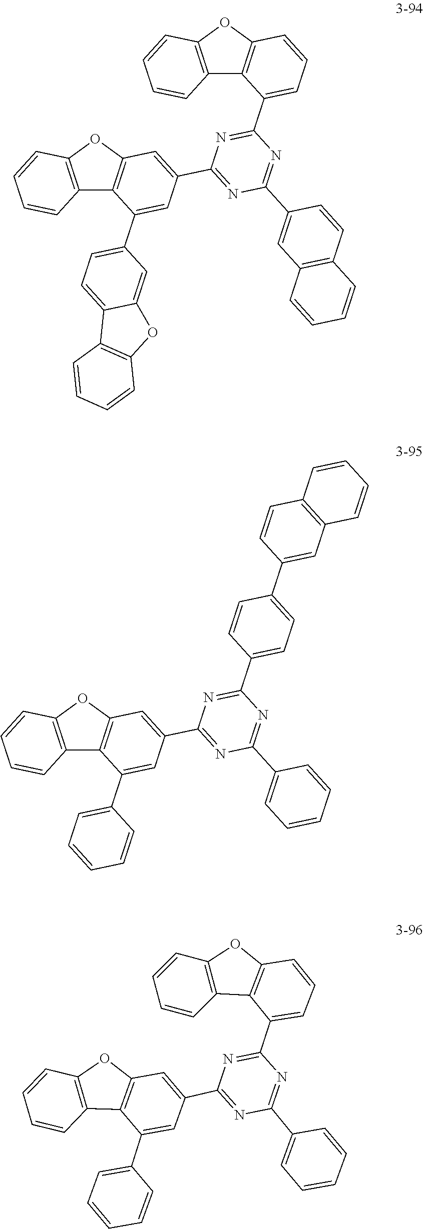 [SMILES: c1ccc2cc(-c3nc(-c4cc(-c5ccc6c(c5)oc5ccccc56)c5c(c4)oc4ccccc45)nc(-c4cccc5oc6ccccc6c45)n3)ccc2c1 ; c1ccc(-c2nc(-c3ccc(-c4ccc5ccccc5c4)cc3)nc(-c3cc(-c4ccccc4)c4c(c3)oc3ccccc34)n2)cc1 ; c1ccc(-c2nc(-c3cc(-c4ccccc4)c4c(c3)oc3ccccc34)nc(-c3cccc4oc5ccccc5c34)n2)cc1]

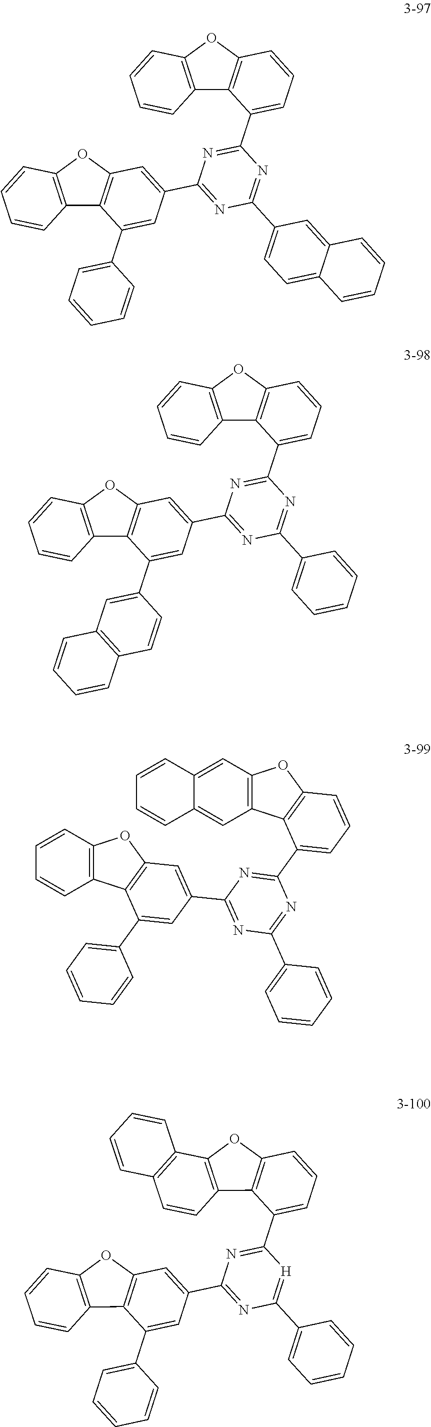

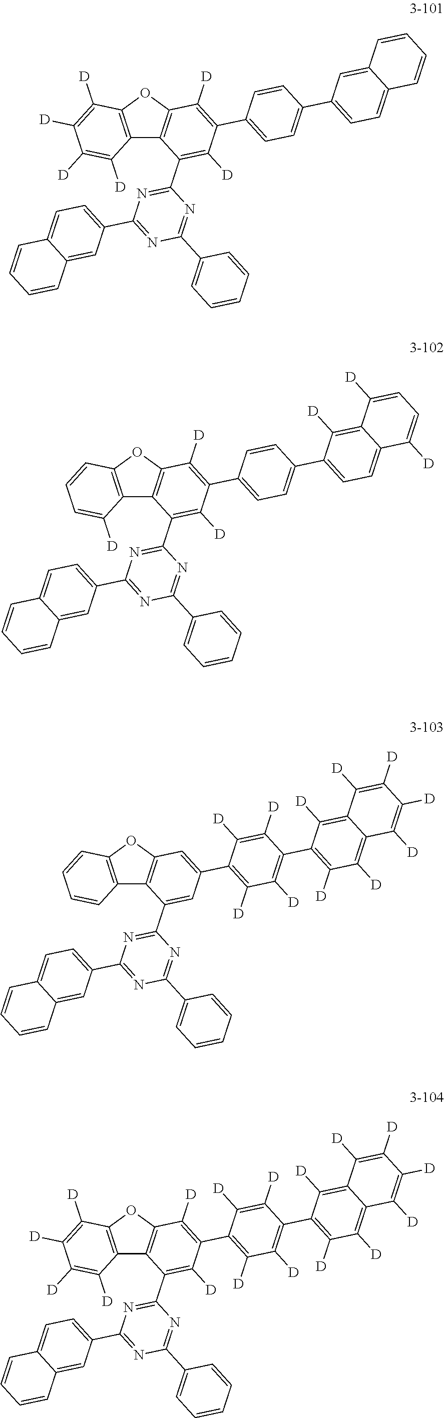

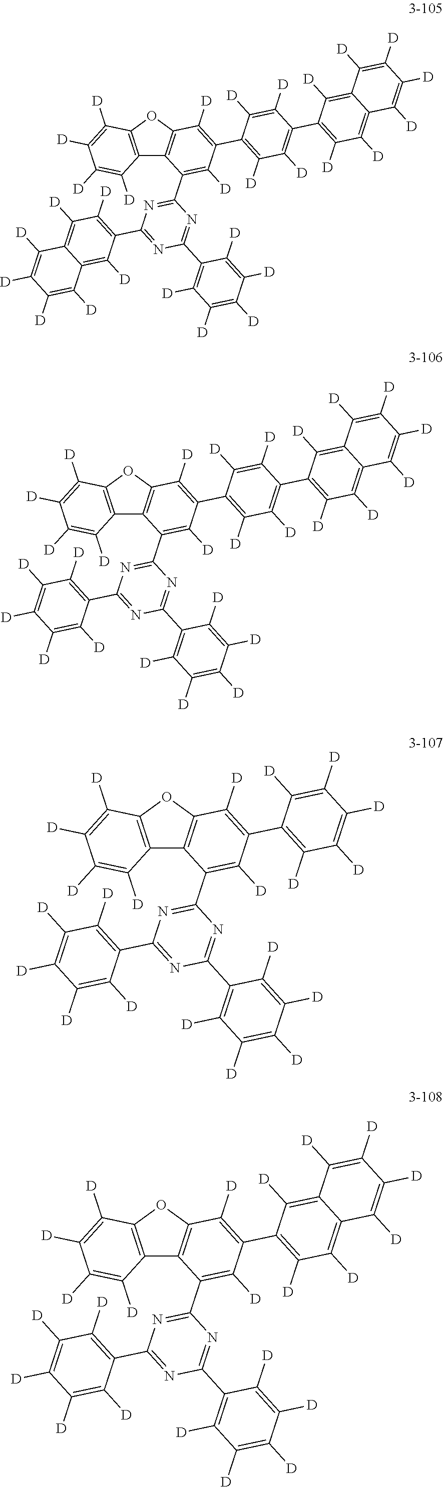

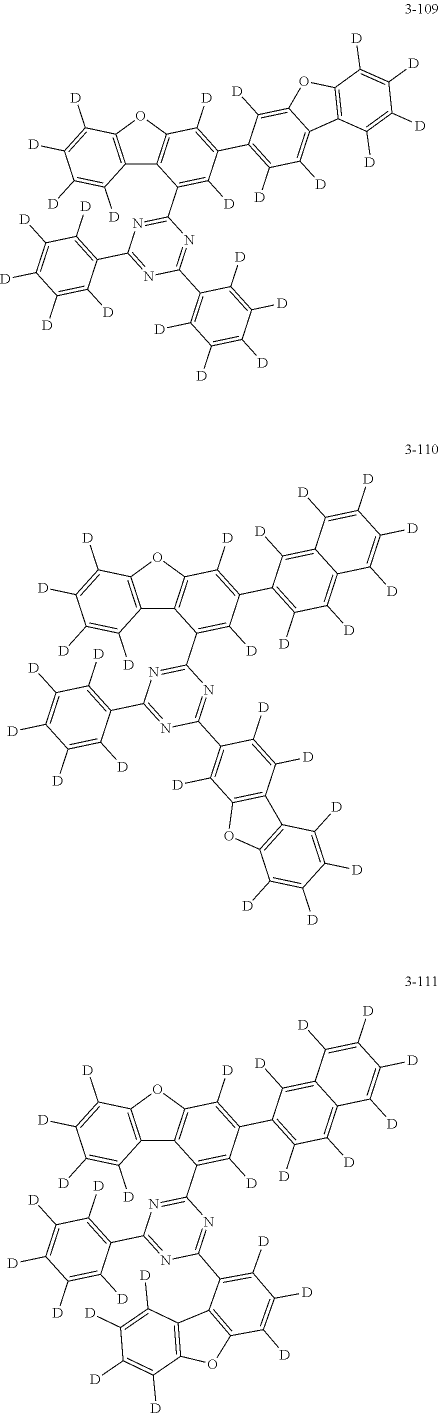

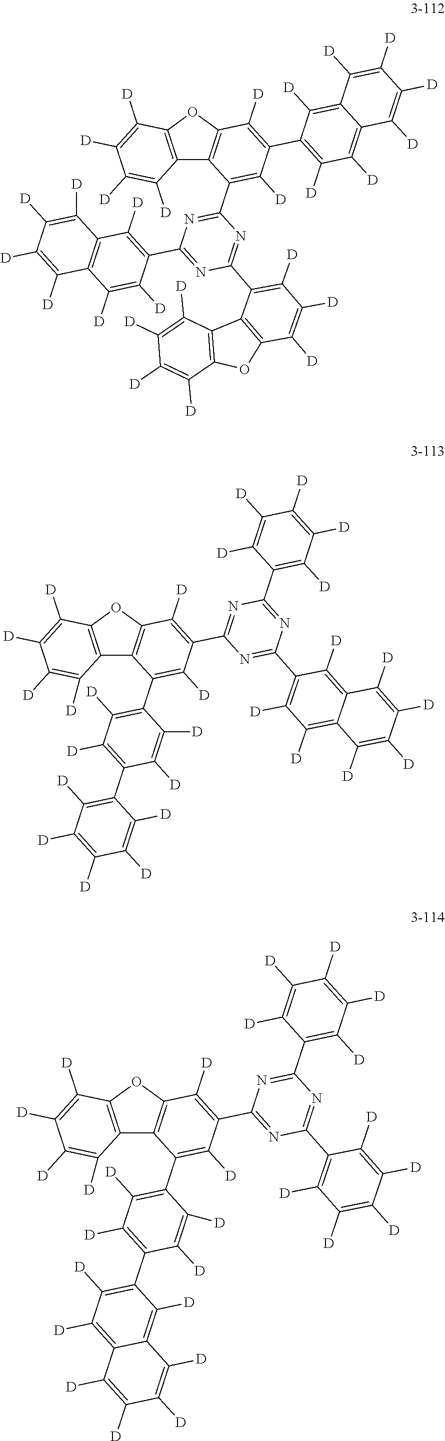

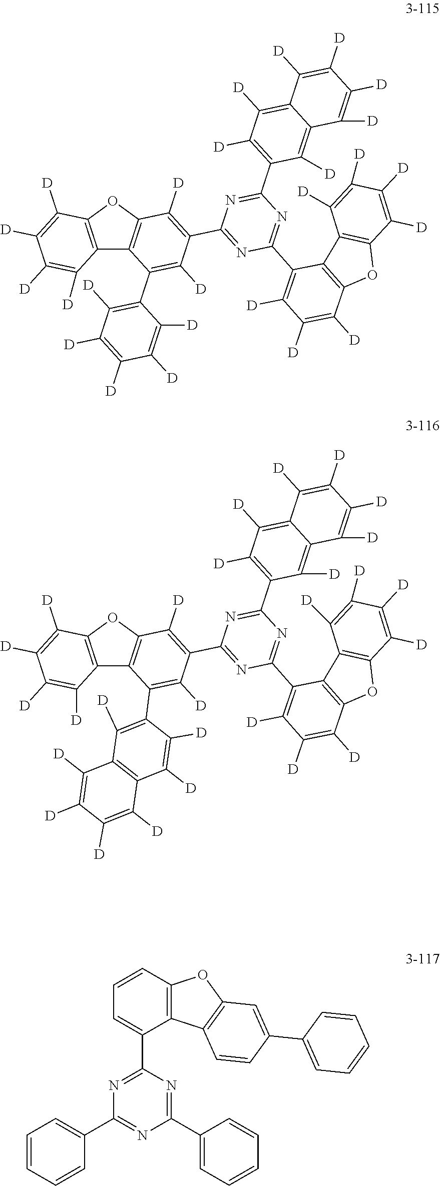

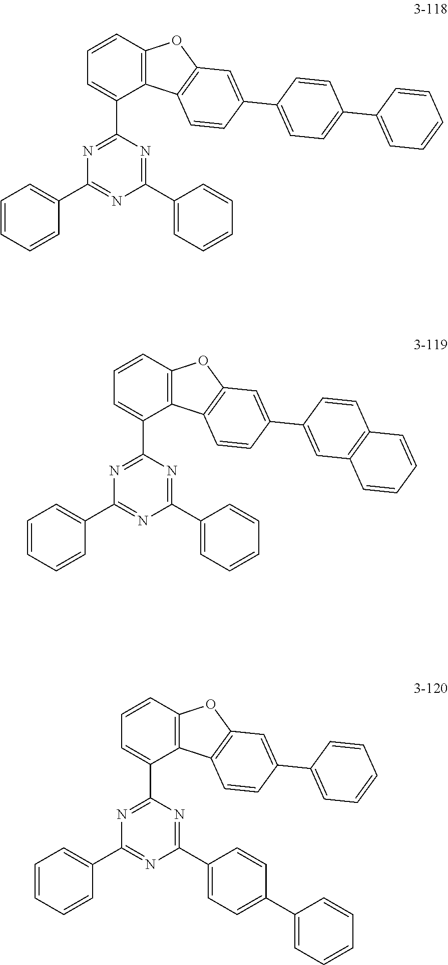 [SMILES: c1ccc(-c2ccc(-c3ccc4c(c3)oc3cccc(-c5nc(-c6ccccc6)nc(-c6ccccc6)n5)c34)cc2)cc1 ; c1ccc(-c2nc(-c3ccccc3)nc(-c3cccc4oc5cc(-c6ccc7ccccc7c6)ccc5c34)n2)cc1 ; c1ccc(-c2ccc(-c3nc(-c4ccccc4)nc(-c4cccc5oc6cc(-c7ccccc7)ccc6c45)n3)cc2)cc1]

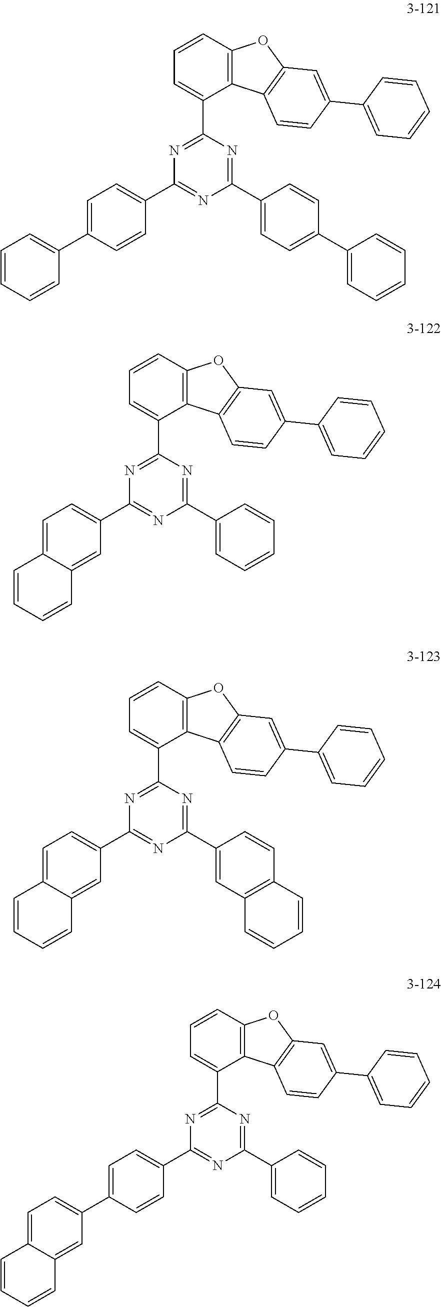 [SMILES: c1ccc(-c2ccc(-c3nc(-c4ccc(-c5ccccc5)cc4)nc(-c4cccc5oc6cc(-c7ccccc7)ccc6c45)n3)cc2)cc1 ; c1ccc(-c2ccc3c(c2)oc2cccc(-c4nc(-c5ccccc5)nc(-c5ccc6ccccc6c5)n4)c23)cc1 ; c1ccc(-c2ccc3c(c2)oc2cccc(-c4nc(-c5ccc6ccccc6c5)nc(-c5ccc6ccccc6c5)n4)c23)cc1 ; c1ccc(-c2ccc3c(c2)oc2cccc(-c4nc(-c5ccccc5)nc(-c5ccc(-c6ccc7ccccc7c6)cc5)n4)c23)cc1]

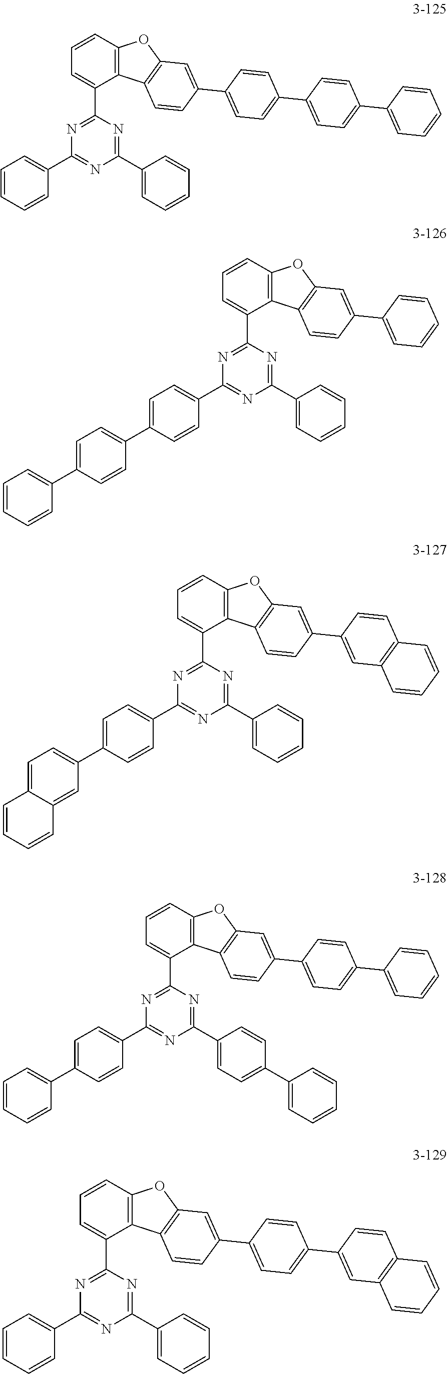 [SMILES: c1ccc(-c2ccc(-c3ccc(-c4ccc5c(c4)oc4cccc(-c6nc(-c7ccccc7)nc(-c7ccccc7)n6)c45)cc3)cc2)cc1 ; c1ccc(-c2ccc(-c3ccc(-c4nc(-c5ccccc5)nc(-c5cccc6oc7cc(-c8ccccc8)ccc7c56)n4)cc3)cc2)cc1 ; c1ccc(-c2nc(-c3ccc(-c4ccc5ccccc5c4)cc3)nc(-c3cccc4oc5cc(-c6ccc7ccccc7c6)ccc5c34)n2)cc1 ; c1ccc(-c2ccc(-c3ccc4c(c3)oc3cccc(-c5nc(-c6ccc(-c7ccccc7)cc6)nc(-c6ccc(-c7ccccc7)cc6)n5)c34)cc2)cc1 ; c1ccc(-c2nc(-c3ccccc3)nc(-c3cccc4oc5cc(-c6ccc(-c7ccc8ccccc8c7)cc6)ccc5c34)n2)cc1]

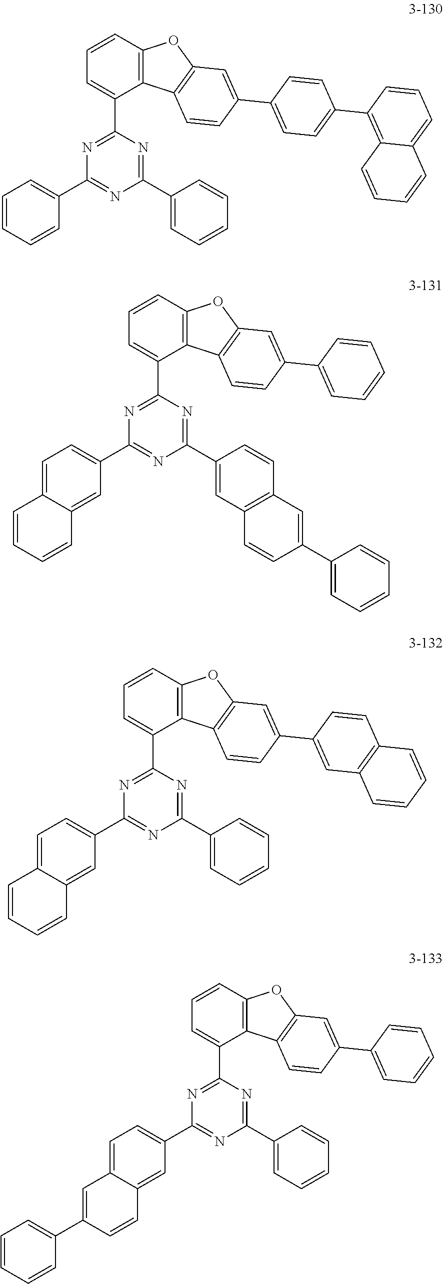 [SMILES: c1ccc(-c2nc(-c3ccccc3)nc(-c3cccc4oc5cc(-c6ccc(-c7cccc8ccccc78)cc6)ccc5c34)n2)cc1 ; c1ccc(-c2ccc3cc(-c4nc(-c5ccc6ccccc6c5)nc(-c5cccc6oc7cc(-c8ccccc8)ccc7c56)n4)ccc3c2)cc1 ; c1ccc(-c2nc(-c3ccc4ccccc4c3)nc(-c3cccc4oc5cc(-c6ccc7ccccc7c6)ccc5c34)n2)cc1 ; c1ccc(-c2ccc3cc(-c4nc(-c5ccccc5)nc(-c5cccc6oc7cc(-c8ccccc8)ccc7c56)n4)ccc3c2)cc1]

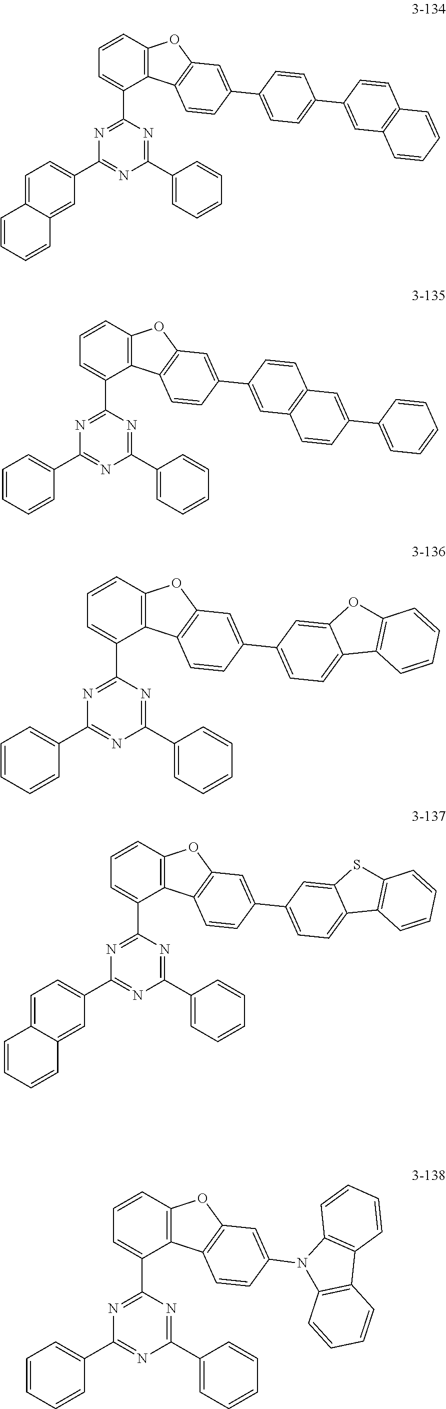 [SMILES: c1ccc(-c2nc(-c3ccc4ccccc4c3)nc(-c3cccc4oc5cc(-c6ccc(-c7ccc8ccccc8c7)cc6)ccc5c34)n2)cc1 ; c1ccc(-c2ccc3cc(-c4ccc5c(c4)oc4cccc(-c6nc(-c7ccccc7)nc(-c7ccccc7)n6)c45)ccc3c2)cc1 ; c1ccc(-c2nc(-c3ccccc3)nc(-c3cccc4oc5cc(-c6ccc7c(c6)oc6ccccc67)ccc5c34)n2)cc1 ; c1ccc(-c2nc(-c3ccc4ccccc4c3)nc(-c3cccc4oc5cc(-c6ccc7c(c6)sc6ccccc67)ccc5c34)n2)cc1 ; c1ccc(-c2nc(-c3ccccc3)nc(-c3cccc4oc5cc(-n6c7ccccc7c7ccccc76)ccc5c34)n2)cc1]

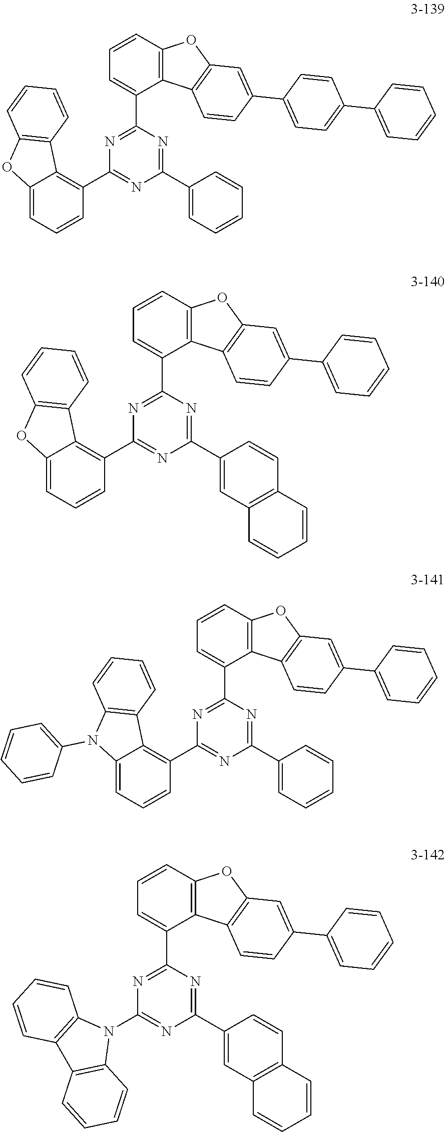 [SMILES: c1ccc(-c2ccc(-c3ccc4c(c3)oc3cccc(-c5nc(-c6ccccc6)nc(-c6cccc7oc8ccccc8c67)n5)c34)cc2)cc1 ; c1ccc(-c2ccc3c(c2)oc2cccc(-c4nc(-c5ccc6ccccc6c5)nc(-c5cccc6oc7ccccc7c56)n4)c23)cc1 ; c1ccc(-c2ccc3c(c2)oc2cccc(-c4nc(-c5ccccc5)nc(-c5cccc6c5c5ccccc5n6-c5ccccc5)n4)c23)cc1 ; c1ccc(-c2ccc3c(c2)oc2cccc(-c4nc(-c5ccc6ccccc6c5)nc(-n5c6ccccc6c6ccccc65)n4)c23)cc1]

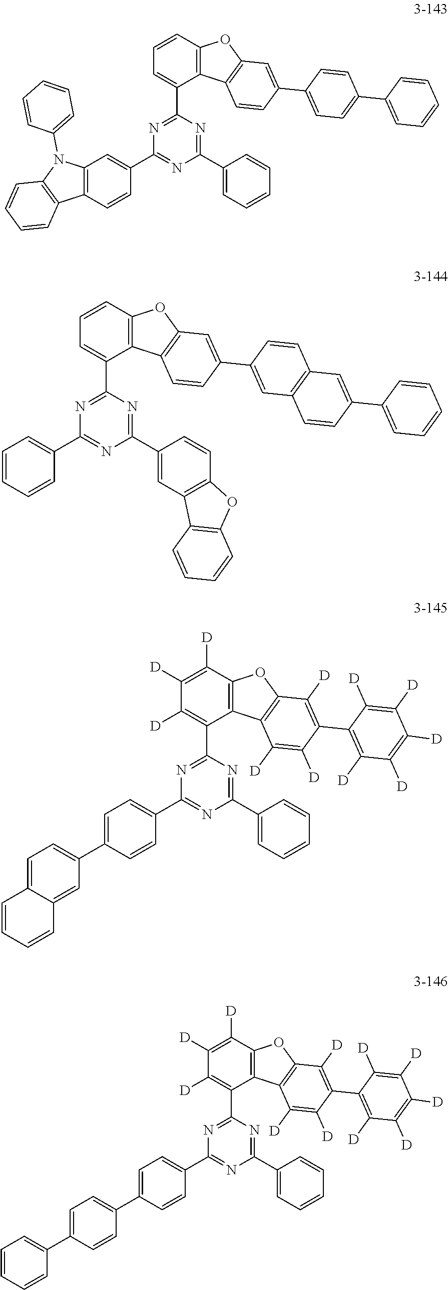 [SMILES: c1ccc(-c2ccc(-c3ccc4c(c3)oc3cccc(-c5nc(-c6ccccc6)nc(-c6ccc7c8ccccc8n(-c8ccccc8)c7c6)n5)c34)cc2)cc1 ; c1ccc(-c2ccc3cc(-c4ccc5c(c4)oc4cccc(-c6nc(-c7ccccc7)nc(-c7ccc8oc9ccccc9c8c7)n6)c45)ccc3c2)cc1 ; [2H]c1c([2H])c([2H])c(-c2c([2H])c([2H])c3c(oc4c([2H])c([2H])c([2H])c(-c5nc(-c6ccccc6)nc(-c6ccc(-c7ccc8ccccc8c7)cc6)n5)c43)c2[2H])c([2H])c1[2H] ; [2H]c1c([2H])c([2H])c(-c2c([2H])c([2H])c3c(oc4c([2H])c([2H])c([2H])c(-c5nc(-c6ccccc6)nc(-c6ccc(-c7ccc(-c8ccccc8)cc7)cc6)n5)c43)c2[2H])c([2H])c1[2H]]

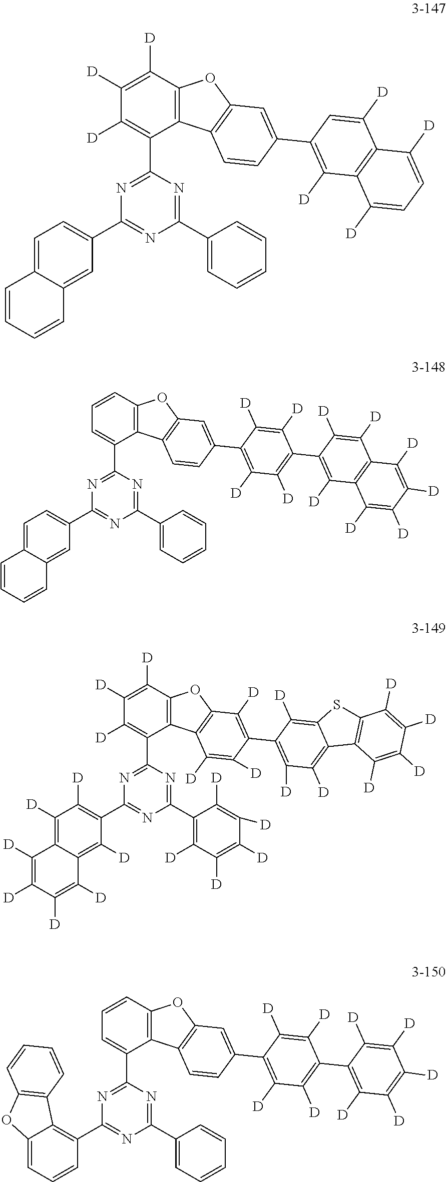

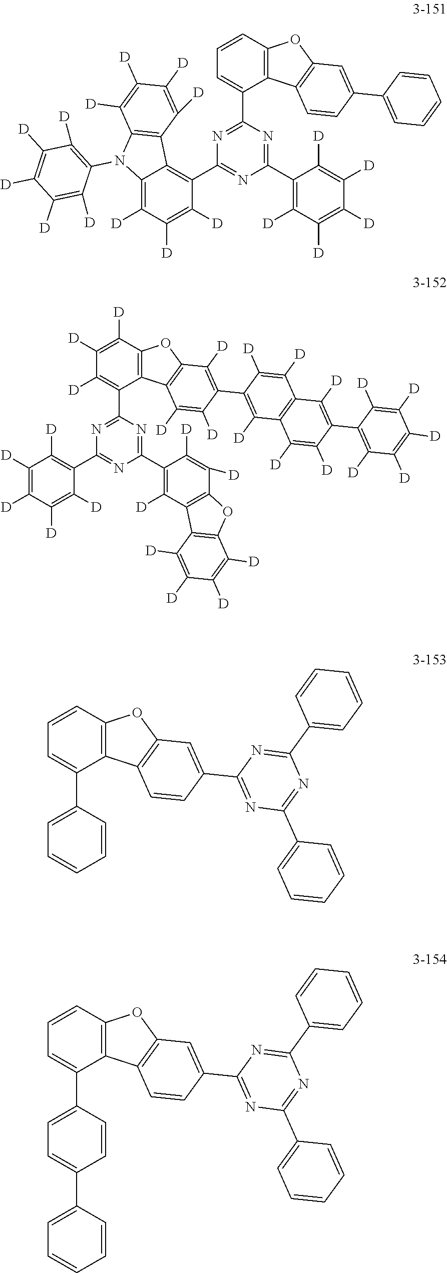

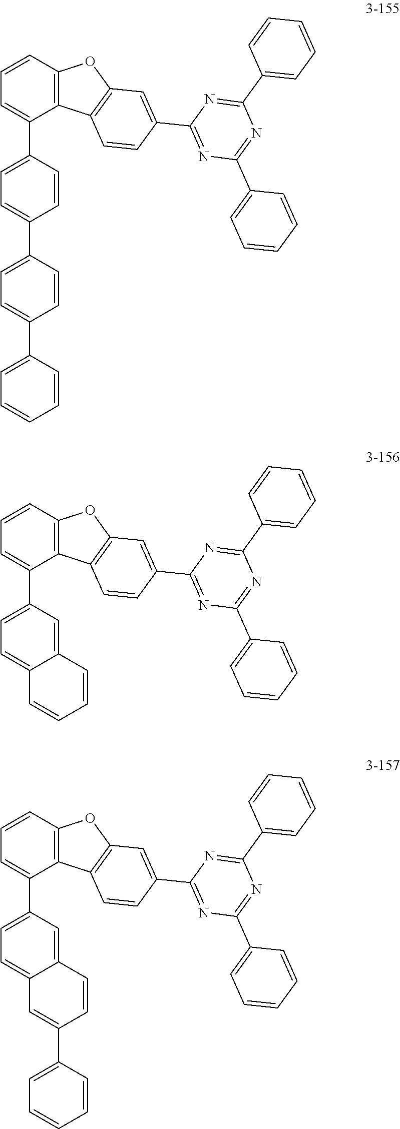 [SMILES: c1ccc(-c2ccc(-c3ccc(-c4cccc5oc6cc(-c7nc(-c8ccccc8)nc(-c8ccccc8)n7)ccc6c45)cc3)cc2)cc1 ; c1ccc(-c2nc(-c3ccccc3)nc(-c3ccc4c(c3)oc3cccc(-c5ccc6ccccc6c5)c34)n2)cc1 ; c1ccc(-c2ccc3cc(-c4cccc5oc6cc(-c7nc(-c8ccccc8)nc(-c8ccccc8)n7)ccc6c45)ccc3c2)cc1]

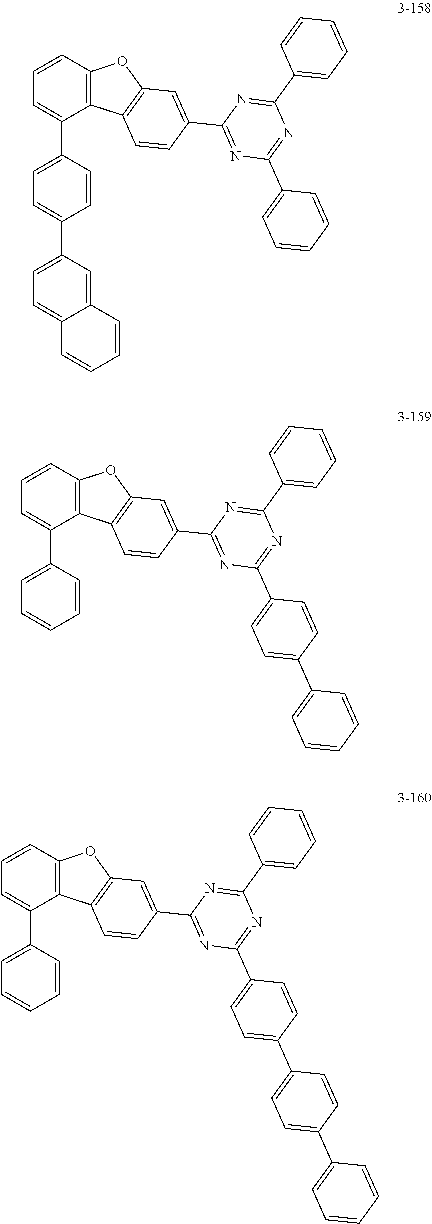 [SMILES: c1ccc(-c2nc(-c3ccccc3)nc(-c3ccc4c(c3)oc3cccc(-c5ccc(-c6ccc7ccccc7c6)cc5)c34)n2)cc1 ; c1ccc(-c2ccc(-c3nc(-c4ccccc4)nc(-c4ccc5c(c4)oc4cccc(-c6ccccc6)c45)n3)cc2)cc1 ; c1ccc(-c2ccc(-c3ccc(-c4nc(-c5ccccc5)nc(-c5ccc6c(c5)oc5cccc(-c7ccccc7)c56)n4)cc3)cc2)cc1]

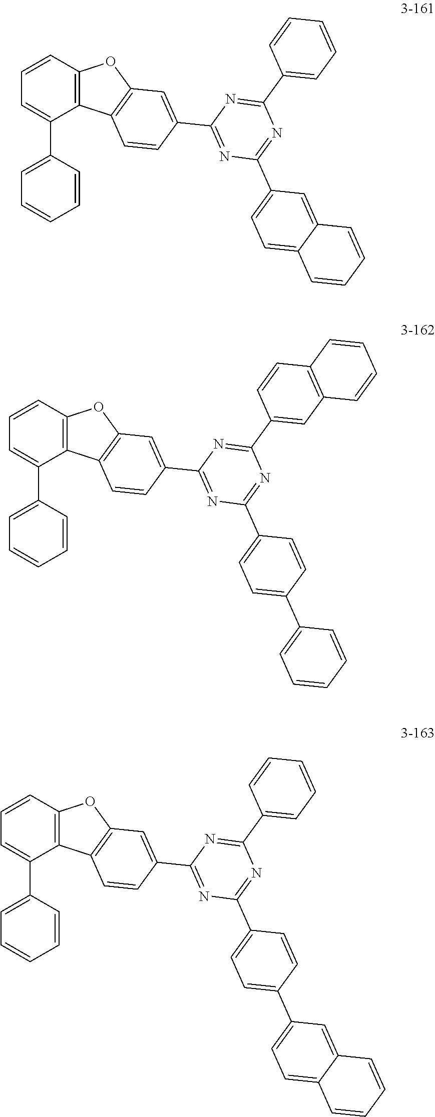 [SMILES: c1ccc(-c2nc(-c3ccc4ccccc4c3)nc(-c3ccc4c(c3)oc3cccc(-c5ccccc5)c34)n2)cc1 ; c1ccc(-c2ccc(-c3nc(-c4ccc5ccccc5c4)nc(-c4ccc5c(c4)oc4cccc(-c6ccccc6)c45)n3)cc2)cc1 ; c1ccc(-c2nc(-c3ccc(-c4ccc5ccccc5c4)cc3)nc(-c3ccc4c(c3)oc3cccc(-c5ccccc5)c34)n2)cc1]

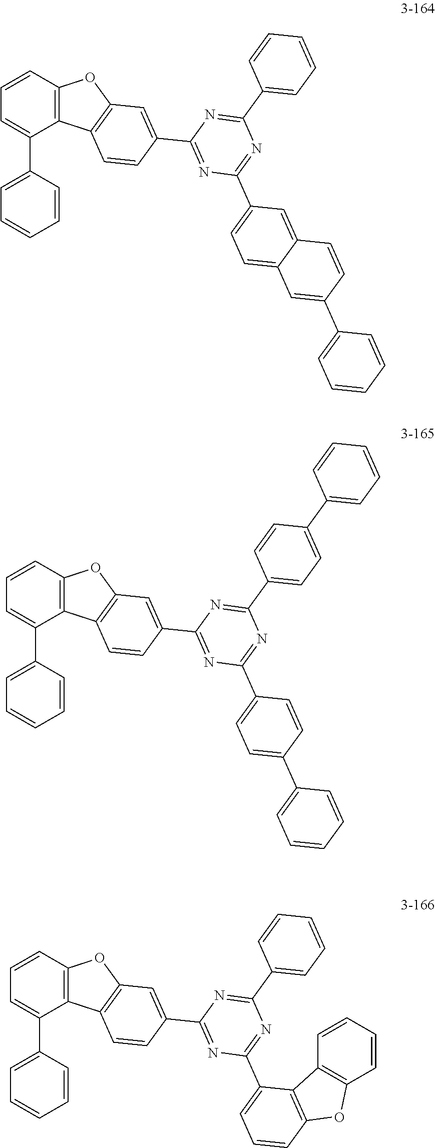 [SMILES: c1ccc(-c2ccc3cc(-c4nc(-c5ccccc5)nc(-c5ccc6c(c5)oc5cccc(-c7ccccc7)c56)n4)ccc3c2)cc1 ; c1ccc(-c2ccc(-c3nc(-c4ccc(-c5ccccc5)cc4)nc(-c4ccc5c(c4)oc4cccc(-c6ccccc6)c45)n3)cc2)cc1 ; c1ccc(-c2nc(-c3ccc4c(c3)oc3cccc(-c5ccccc5)c34)nc(-c3cccc4oc5ccccc5c34)n2)cc1]

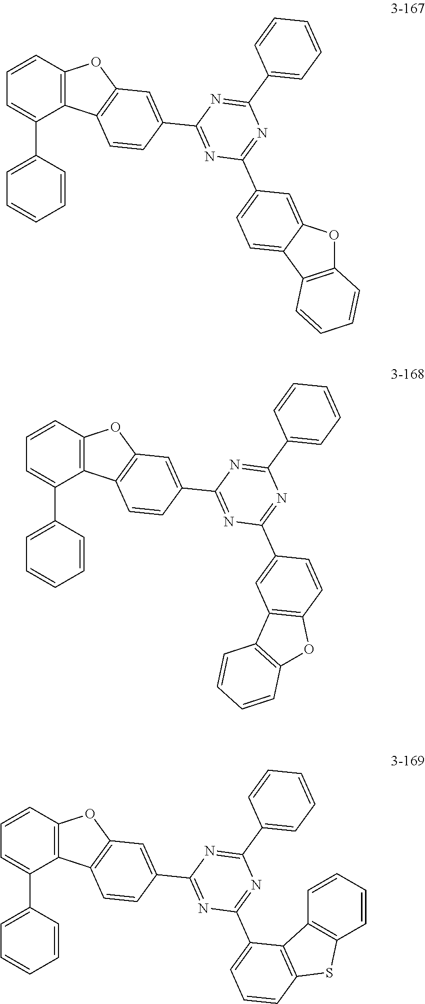 [SMILES: c1ccc(-c2nc(-c3ccc4c(c3)oc3ccccc34)nc(-c3ccc4c(c3)oc3cccc(-c5ccccc5)c34)n2)cc1 ; c1ccc(-c2nc(-c3ccc4c(c3)oc3cccc(-c5ccccc5)c34)nc(-c3ccc4oc5ccccc5c4c3)n2)cc1 ; c1ccc(-c2nc(-c3ccc4c(c3)oc3cccc(-c5ccccc5)c34)nc(-c3cccc4sc5ccccc5c34)n2)cc1]

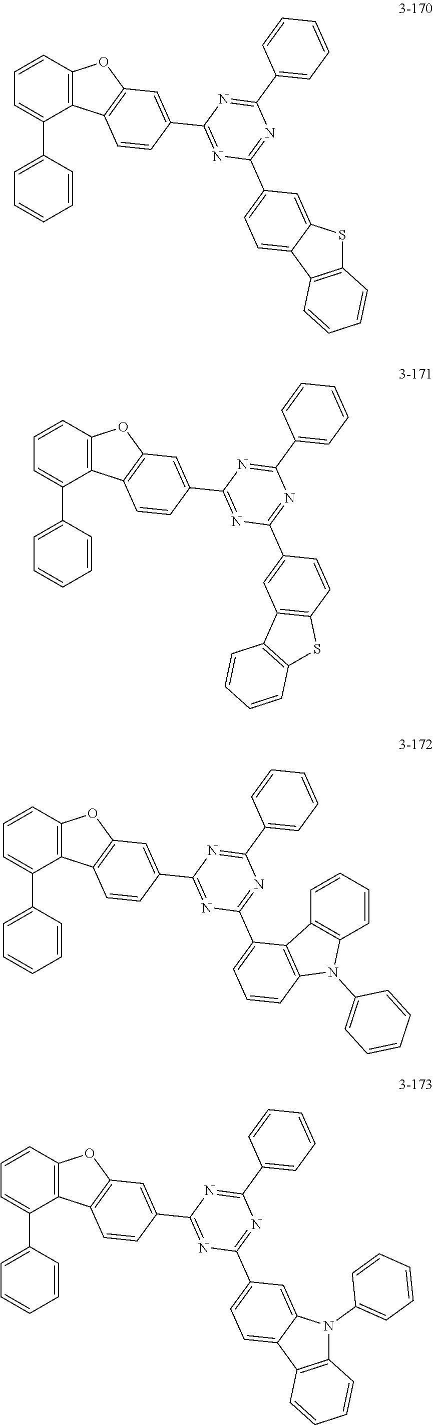 [SMILES: c1ccc(-c2nc(-c3ccc4c(c3)oc3cccc(-c5ccccc5)c34)nc(-c3ccc4c(c3)sc3ccccc34)n2)cc1 ; c1ccc(-c2nc(-c3ccc4c(c3)oc3cccc(-c5ccccc5)c34)nc(-c3ccc4sc5ccccc5c4c3)n2)cc1 ; c1ccc(-c2nc(-c3ccc4c(c3)oc3cccc(-c5ccccc5)c34)nc(-c3cccc4c3c3ccccc3n4-c3ccccc3)n2)cc1 ; c1ccc(-c2nc(-c3ccc4c(c3)oc3cccc(-c5ccccc5)c34)nc(-c3ccc4c5ccccc5n(-c5ccccc5)c4c3)n2)cc1]

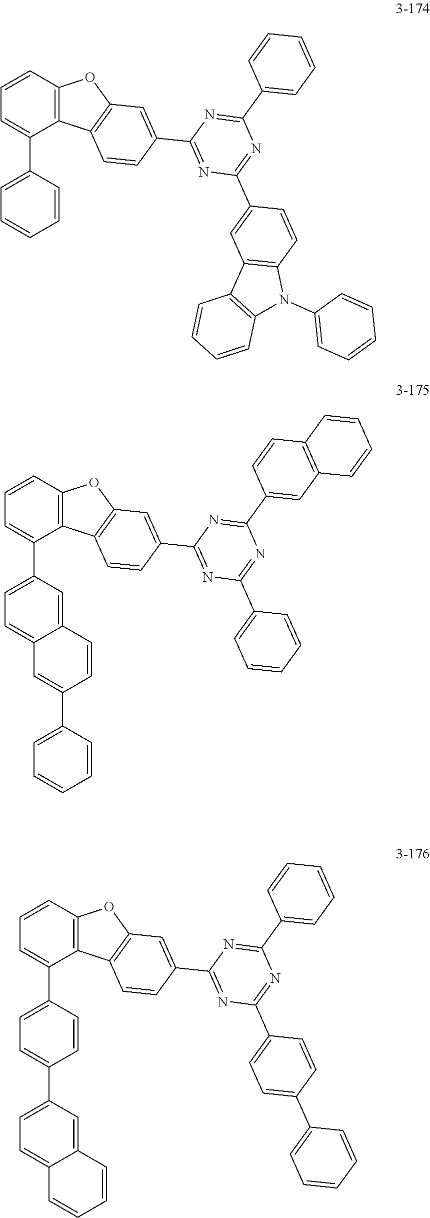 [SMILES: c1ccc(-c2nc(-c3ccc4c(c3)oc3cccc(-c5ccccc5)c34)nc(-c3ccc4c(c3)c3ccccc3n4-c3ccccc3)n2)cc1 ; c1ccc(-c2ccc3cc(-c4cccc5oc6cc(-c7nc(-c8ccccc8)nc(-c8ccc9ccccc9c8)n7)ccc6c45)ccc3c2)cc1 ; c1ccc(-c2ccc(-c3nc(-c4ccccc4)nc(-c4ccc5c(c4)oc4cccc(-c6ccc(-c7ccc8ccccc8c7)cc6)c45)n3)cc2)cc1]

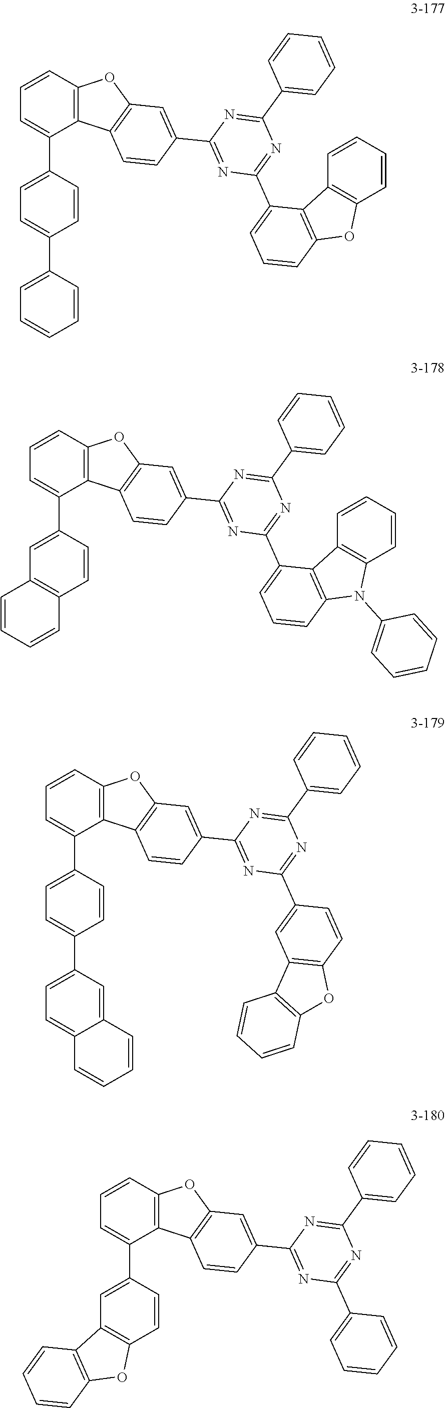 [SMILES: c1ccc(-c2ccc(-c3cccc4oc5cc(-c6nc(-c7ccccc7)nc(-c7cccc8oc9ccccc9c78)n6)ccc5c34)cc2)cc1 ; c1ccc(-c2nc(-c3ccc4c(c3)oc3cccc(-c5ccc6ccccc6c5)c34)nc(-c3cccc4c3c3ccccc3n4-c3ccccc3)n2)cc1 ; c1ccc(-c2nc(-c3ccc4c(c3)oc3cccc(-c5ccc(-c6ccc7ccccc7c6)cc5)c34)nc(-c3ccc4oc5ccccc5c4c3)n2)cc1 ; c1ccc(-c2nc(-c3ccccc3)nc(-c3ccc4c(c3)oc3cccc(-c5ccc6oc7ccccc7c6c5)c34)n2)cc1]

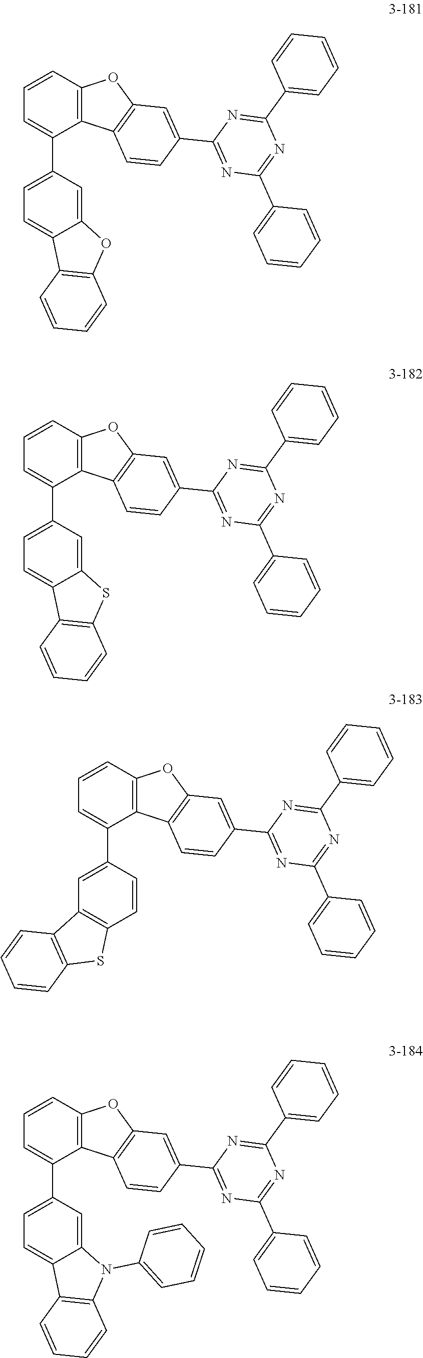 [SMILES: c1ccc(-c2nc(-c3ccccc3)nc(-c3ccc4c(c3)oc3cccc(-c5ccc6c(c5)oc5ccccc56)c34)n2)cc1 ; c1ccc(-c2nc(-c3ccccc3)nc(-c3ccc4c(c3)oc3cccc(-c5ccc6c(c5)sc5ccccc56)c34)n2)cc1 ; c1ccc(-c2nc(-c3ccccc3)nc(-c3ccc4c(c3)oc3cccc(-c5ccc6sc7ccccc7c6c5)c34)n2)cc1 ; c1ccc(-c2nc(-c3ccccc3)nc(-c3ccc4c(c3)oc3cccc(-c5ccc6c7ccccc7n(-c7ccccc7)c6c5)c34)n2)cc1]

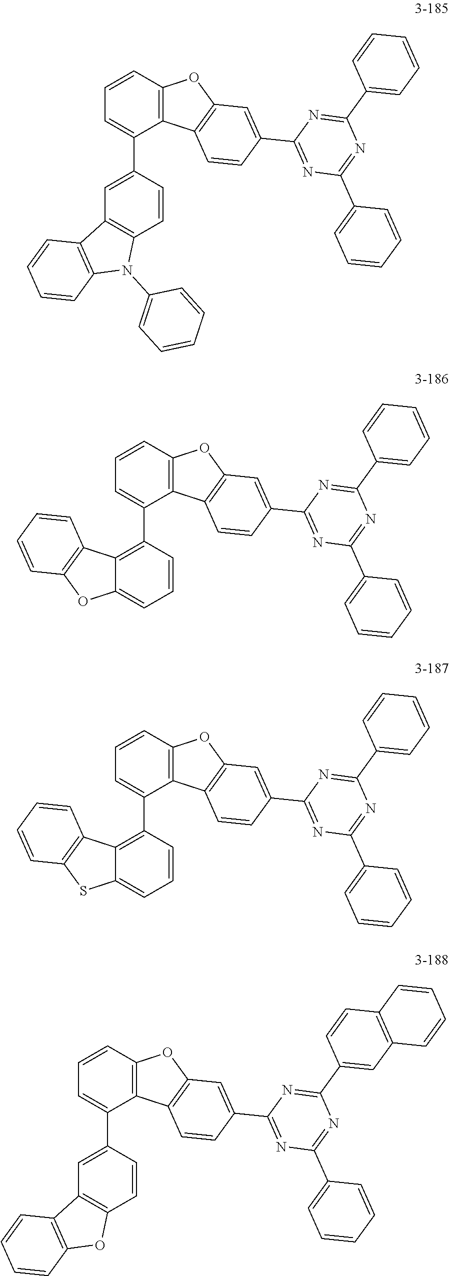 [SMILES: c1ccc(-c2nc(-c3ccccc3)nc(-c3ccc4c(c3)oc3cccc(-c5ccc6c(c5)c5ccccc5n6-c5ccccc5)c34)n2)cc1 ; c1ccc(-c2nc(-c3ccccc3)nc(-c3ccc4c(c3)oc3cccc(-c5cccc6oc7ccccc7c56)c34)n2)cc1 ; c1ccc(-c2nc(-c3ccccc3)nc(-c3ccc4c(c3)oc3cccc(-c5cccc6sc7ccccc7c56)c34)n2)cc1 ; c1ccc(-c2nc(-c3ccc4ccccc4c3)nc(-c3ccc4c(c3)oc3cccc(-c5ccc6oc7ccccc7c6c5)c34)n2)cc1]

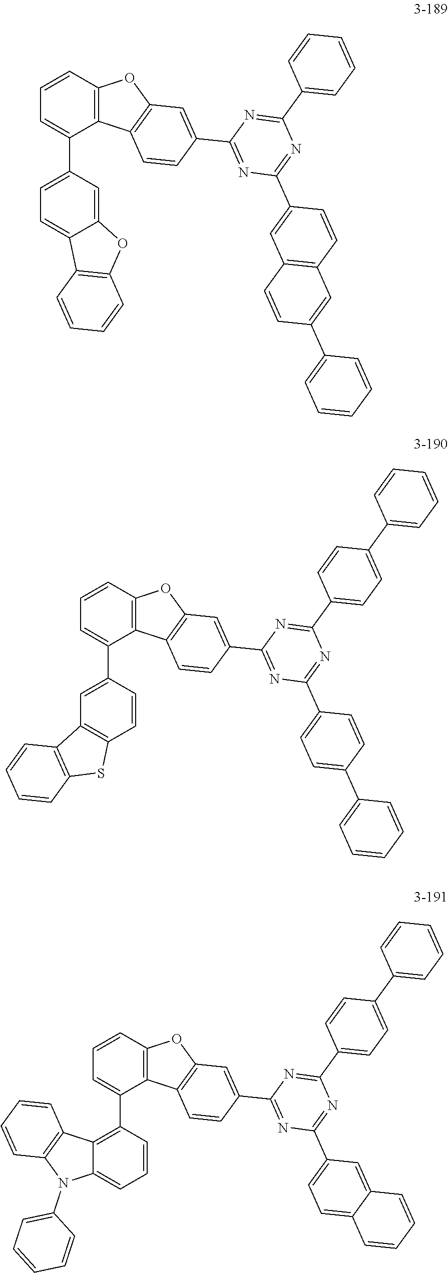 [SMILES: c1ccc(-c2ccc3cc(-c4nc(-c5ccccc5)nc(-c5ccc6c(c5)oc5cccc(-c7ccc8c(c7)oc7ccccc78)c56)n4)ccc3c2)cc1 ; c1ccc(-c2ccc(-c3nc(-c4ccc(-c5ccccc5)cc4)nc(-c4ccc5c(c4)oc4cccc(-c6ccc7sc8ccccc8c7c6)c45)n3)cc2)cc1 ; c1ccc(-c2ccc(-c3nc(-c4ccc5ccccc5c4)nc(-c4ccc5c(c4)oc4cccc(-c6cccc7c6c6ccccc6n7-c6ccccc6)c45)n3)cc2)cc1]

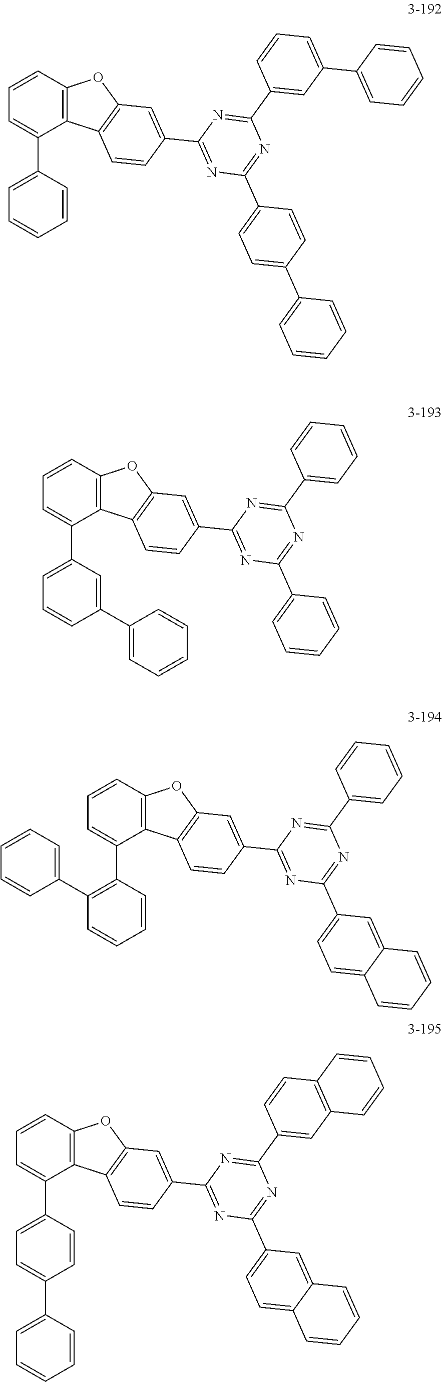 [SMILES: c1ccc(-c2ccc(-c3nc(-c4cccc(-c5ccccc5)c4)nc(-c4ccc5c(c4)oc4cccc(-c6ccccc6)c45)n3)cc2)cc1 ; c1ccc(-c2cccc(-c3cccc4oc5cc(-c6nc(-c7ccccc7)nc(-c7ccccc7)n6)ccc5c34)c2)cc1 ; c1ccc(-c2nc(-c3ccc4ccccc4c3)nc(-c3ccc4c(c3)oc3cccc(-c5ccccc5-c5ccccc5)c34)n2)cc1 ; c1ccc(-c2ccc(-c3cccc4oc5cc(-c6nc(-c7ccc8ccccc8c7)nc(-c7ccc8ccccc8c7)n6)ccc5c34)cc2)cc1]

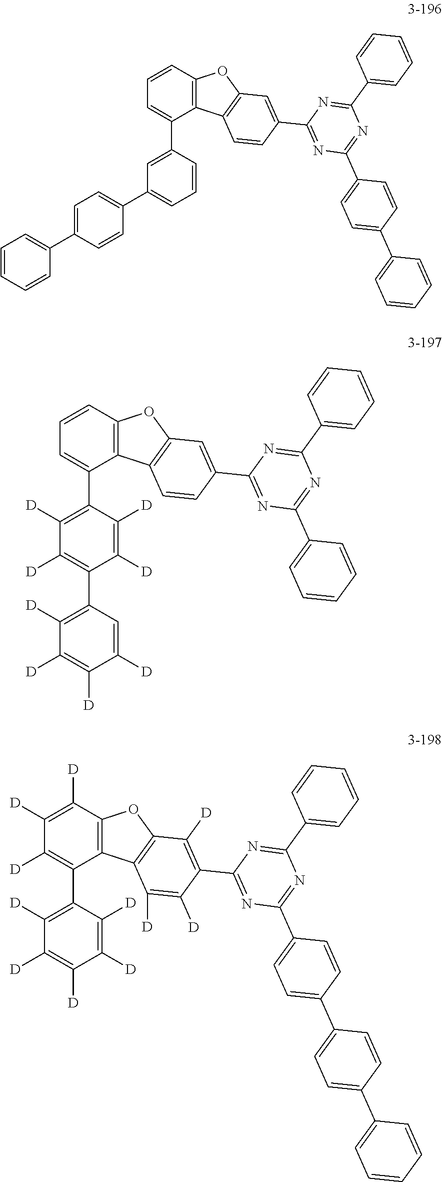 [SMILES: c1ccc(-c2ccc(-c3cccc(-c4cccc5oc6cc(-c7nc(-c8ccccc8)nc(-c8ccc(-c9ccccc9)cc8)n7)ccc6c45)c3)cc2)cc1 ; [2H]c1cc(-c2c([2H])c([2H])c(-c3cccc4oc5cc(-c6nc(-c7ccccc7)nc(-c7ccccc7)n6)ccc5c34)c([2H])c2[2H])c([2H])c([2H])c1[2H] ; [2H]c1c([2H])c([2H])c(-c2c([2H])c([2H])c([2H])c3oc4c([2H])c(-c5nc(-c6ccccc6)nc(-c6ccc(-c7ccc(-c8ccccc8)cc7)cc6)n5)c([2H])c([2H])c4c23)c([2H])c1[2H]]

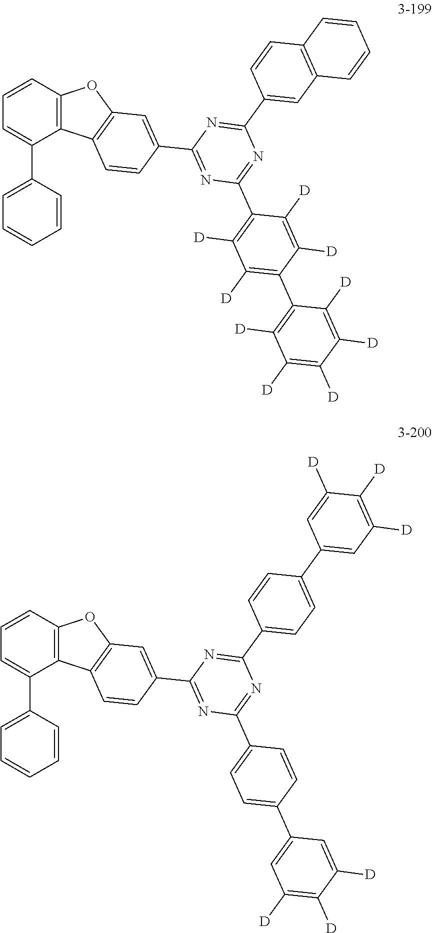 [SMILES: [2H]c1c([2H])c([2H])c(-c2c([2H])c([2H])c(-c3nc(-c4ccc5ccccc5c4)nc(-c4ccc5c(c4)oc4cccc(-c6ccccc6)c45)n3)c([2H])c2[2H])c([2H])c1[2H] ; [2H]c1cc(-c2ccc(-c3nc(-c4ccc(-c5cc([2H])c([2H])c([2H])c5)cc4)nc(-c4ccc5c(c4)oc4cccc(-c6ccccc6)c45)n3)cc2)cc([2H])c1[2H]]

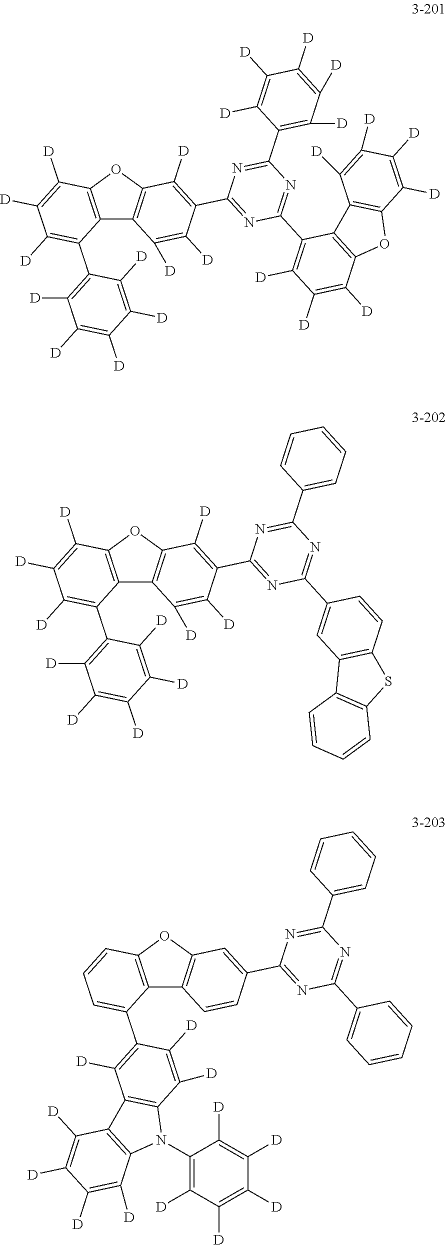

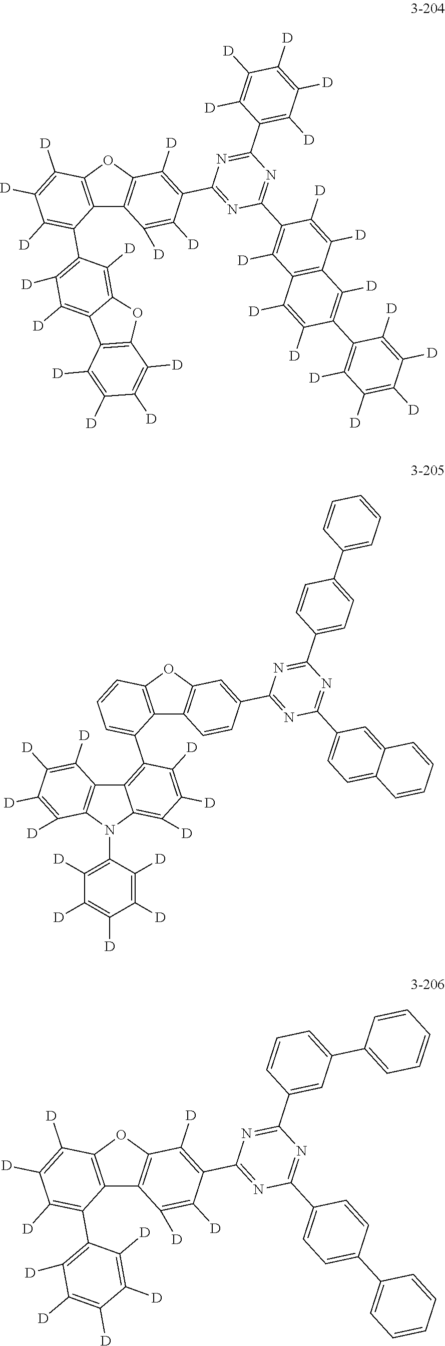

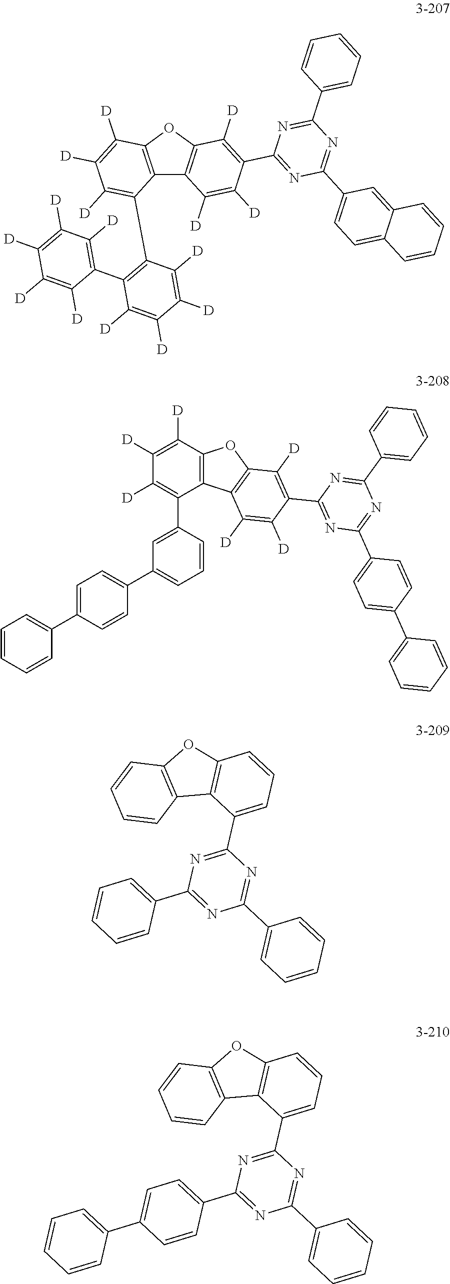 [SMILES: [2H]c1c([2H])c([2H])c(-c2c([2H])c([2H])c([2H])c([2H])c2-c2c([2H])c([2H])c([2H])c3oc4c([2H])c(-c5nc(-c6ccccc6)nc(-c6ccc7ccccc7c6)n5)c([2H])c([2H])c4c23)c([2H])c1[2H] ; [2H]c1c([2H])c(-c2cccc(-c3ccc(-c4ccccc4)cc3)c2)c2c(oc3c([2H])c(-c4nc(-c5ccccc5)nc(-c5ccc(-c6ccccc6)cc5)n4)c([2H])c([2H])c32)c1[2H] ; c1ccc(-c2nc(-c3ccccc3)nc(-c3cccc4oc5ccccc5c34)n2)cc1 ; c1ccc(-c2ccc(-c3nc(-c4ccccc4)nc(-c4cccc5oc6ccccc6c45)n3)cc2)cc1]

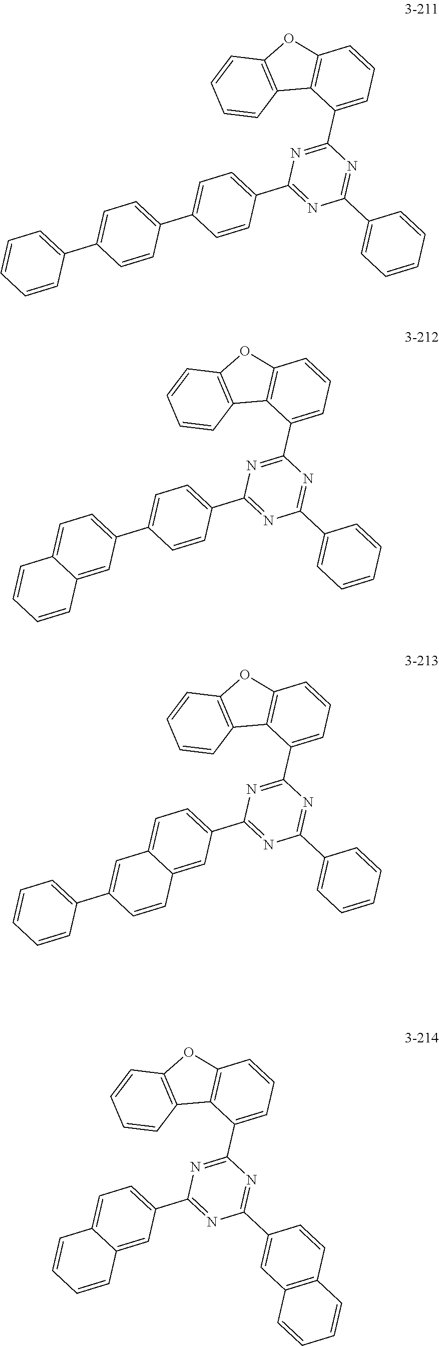 [SMILES: c1ccc(-c2ccc(-c3ccc(-c4nc(-c5ccccc5)nc(-c5cccc6oc7ccccc7c56)n4)cc3)cc2)cc1 ; c1ccc(-c2nc(-c3ccc(-c4ccc5ccccc5c4)cc3)nc(-c3cccc4oc5ccccc5c34)n2)cc1 ; c1ccc(-c2ccc3cc(-c4nc(-c5ccccc5)nc(-c5cccc6oc7ccccc7c56)n4)ccc3c2)cc1 ; c1ccc2cc(-c3nc(-c4ccc5ccccc5c4)nc(-c4cccc5oc6ccccc6c45)n3)ccc2c1]

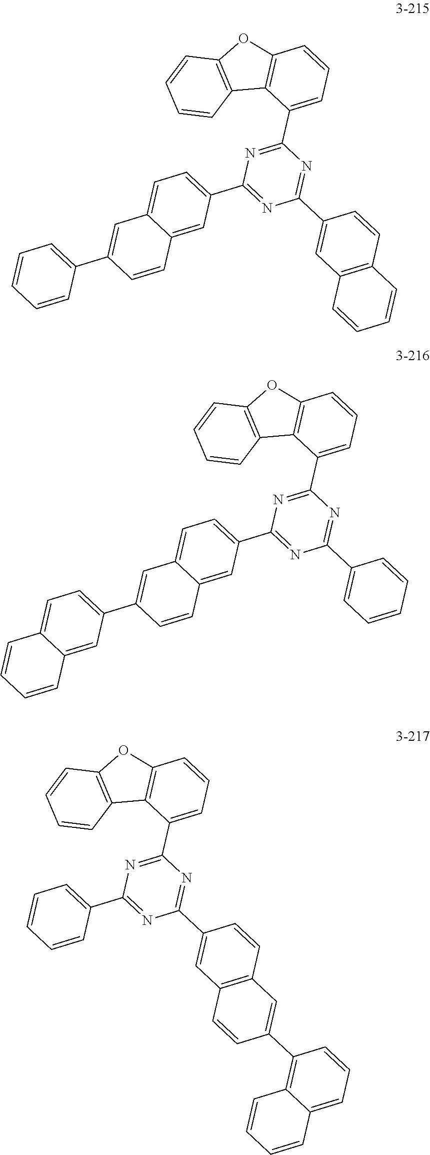 [SMILES: c1ccc(-c2ccc3cc(-c4nc(-c5ccc6ccccc6c5)nc(-c5cccc6oc7ccccc7c56)n4)ccc3c2)cc1 ; c1ccc(-c2nc(-c3ccc4cc(-c5ccc6ccccc6c5)ccc4c3)nc(-c3cccc4oc5ccccc5c34)n2)cc1 ; c1ccc(-c2nc(-c3ccc4cc(-c5cccc6ccccc56)ccc4c3)nc(-c3cccc4oc5ccccc5c34)n2)cc1]

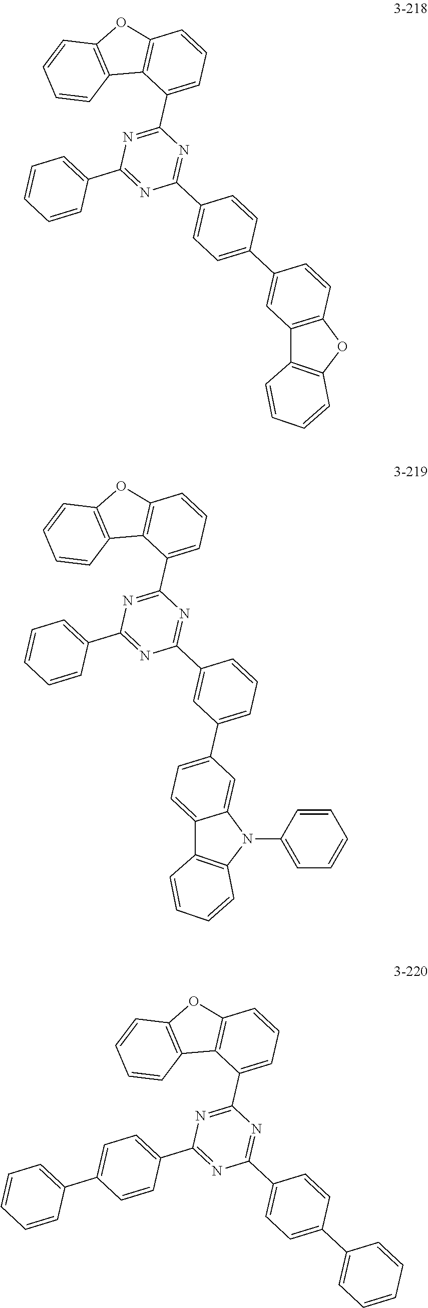 [SMILES: c1ccc(-c2nc(-c3ccc(-c4ccc5oc6ccccc6c5c4)cc3)nc(-c3cccc4oc5ccccc5c34)n2)cc1 ; c1ccc(-c2nc(-c3cccc(-c4ccc5c6ccccc6n(-c6ccccc6)c5c4)c3)nc(-c3cccc4oc5ccccc5c34)n2)cc1 ; c1ccc(-c2ccc(-c3nc(-c4ccc(-c5ccccc5)cc4)nc(-c4cccc5oc6ccccc6c45)n3)cc2)cc1]

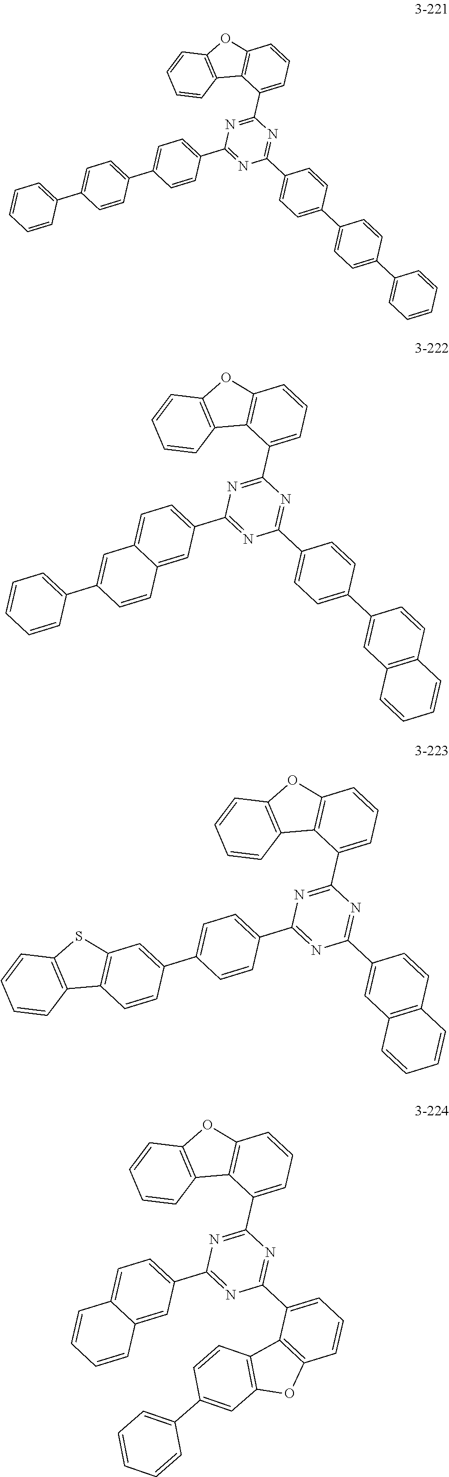 [SMILES: c1ccc(-c2ccc(-c3ccc(-c4nc(-c5ccc(-c6ccc(-c7ccccc7)cc6)cc5)nc(-c5cccc6oc7ccccc7c56)n4)cc3)cc2)cc1 ; c1ccc(-c2ccc3cc(-c4nc(-c5ccc(-c6ccc7ccccc7c6)cc5)nc(-c5cccc6oc7ccccc7c56)n4)ccc3c2)cc1 ; c1ccc2cc(-c3nc(-c4ccc(-c5ccc6c(c5)sc5ccccc56)cc4)nc(-c4cccc5oc6ccccc6c45)n3)ccc2c1 ; c1ccc(-c2ccc3c(c2)oc2cccc(-c4nc(-c5ccc6ccccc6c5)nc(-c5cccc6oc7ccccc7c56)n4)c23)cc1]

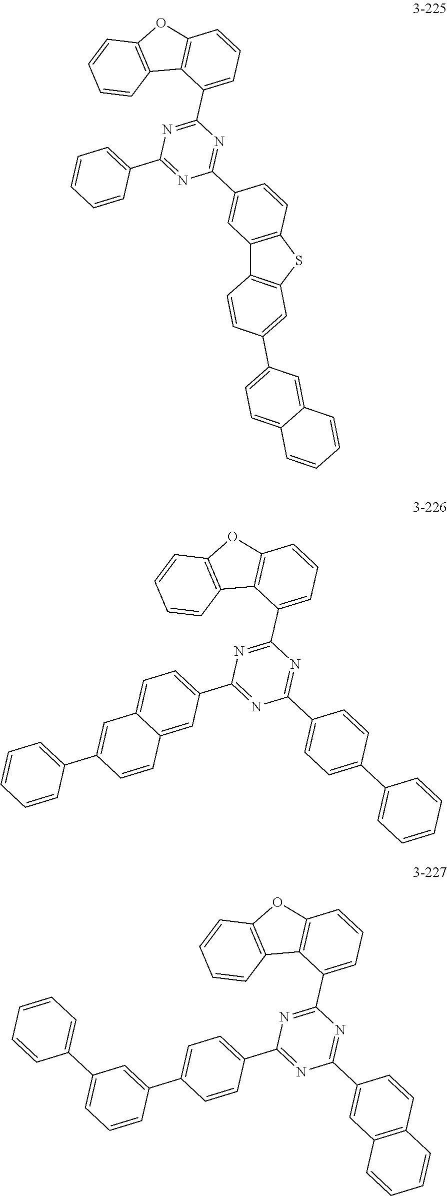 [SMILES: c1ccc(-c2nc(-c3ccc4sc5cc(-c6ccc7ccccc7c6)ccc5c4c3)nc(-c3cccc4oc5ccccc5c34)n2)cc1 ; c1ccc(-c2ccc(-c3nc(-c4ccc5cc(-c6ccccc6)ccc5c4)nc(-c4cccc5oc6ccccc6c45)n3)cc2)cc1 ; c1ccc(-c2cccc(-c3ccc(-c4nc(-c5ccc6ccccc6c5)nc(-c5cccc6oc7ccccc7c56)n4)cc3)c2)cc1]

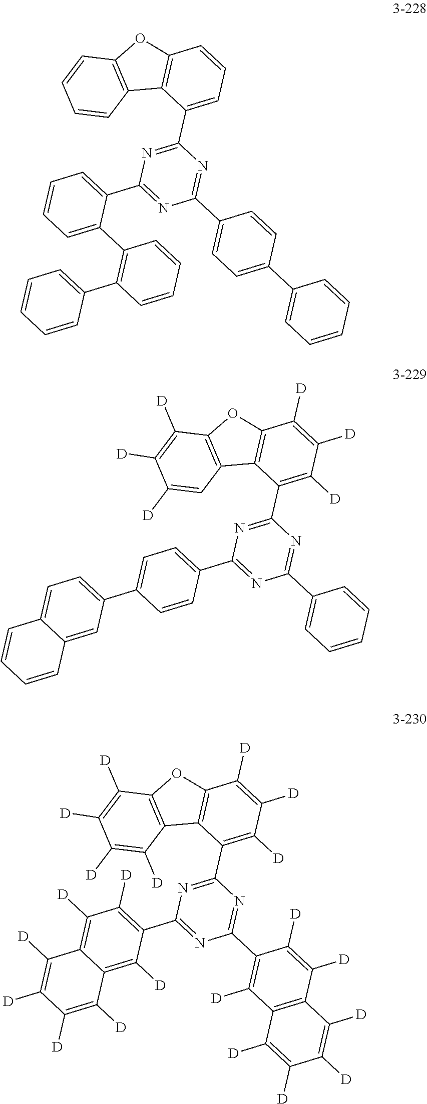

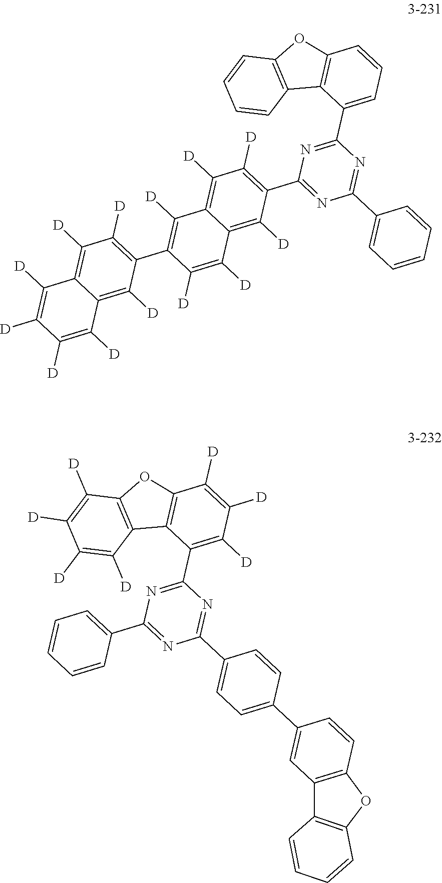 [SMILES: [2H]c1c([2H])c([2H])c2c([2H])c(-c3c([2H])c([2H])c4c([2H])c(-c5nc(-c6ccccc6)nc(-c6cccc7oc8ccccc8c67)n5)c([2H])c([2H])c4c3[2H])c([2H])c([2H])c2c1[2H] ; [2H]c1c([2H])c([2H])c2c(oc3c([2H])c([2H])c([2H])c(-c4nc(-c5ccccc5)nc(-c5ccc(-c6ccc7oc8ccccc8c7c6)cc5)n4)c32)c1[2H]]

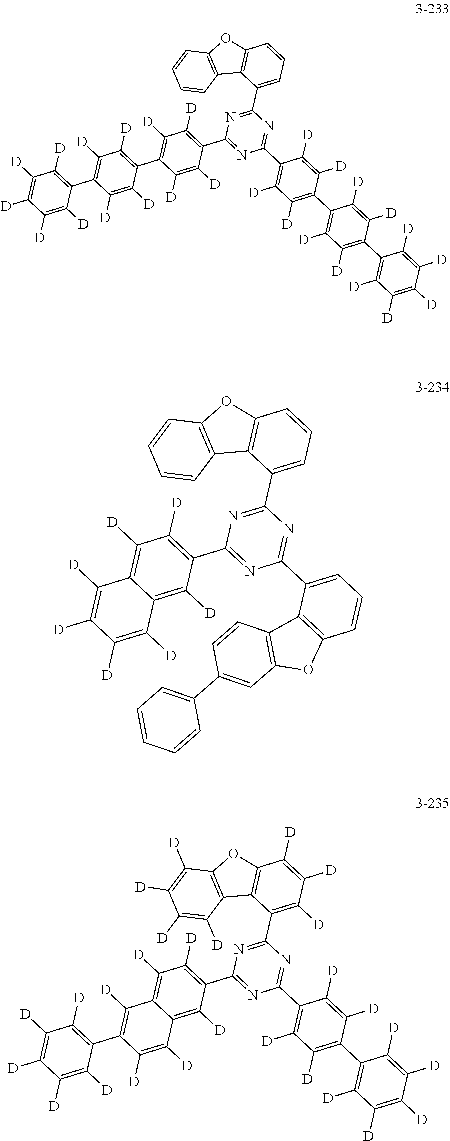

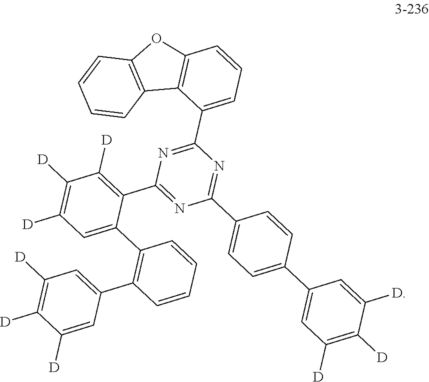

13. The organic light emitting device of claim 9, wherein the organic material layer includes a light emitting layer, and the light emitting layer includes the heterocyclic compound and the heterocyclic compound represented by Chemical Formula 2.

14. The organic light emitting device of claim 7, further comprising one, or two or more layers selected from the group consisting of a light emitting layer, a hole injection layer, a hole transport layer, an electron injection layer, an electron transport layer, an electron blocking layer and a hole blocking layer.

15. A composition for an organic material layer, the composition comprising:
the heterocyclic compound of claim 1; and
a heterocyclic compound represented by the following Chemical Formula 2: 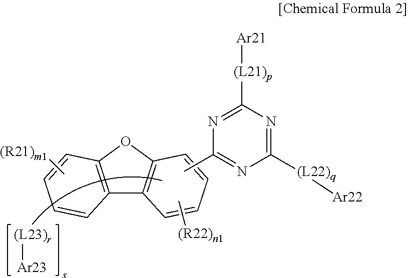
wherein, in Chemical Formula 2,
R21 and R22 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; —P(═O)R201R202; —SiR201R202R203; and —NR201R202, or two or more groups adjacent to each other bond to each other to form a substituted or unsubstituted C6 to C60 aromatic hydrocarbon ring; or a substituted or unsubstituted C2 to C60 heteroring, and R201, R202 and R203 are the same as or different from each other and each independently a substituted or unsubstituted C1 to C60 alkyl group; a substituted or unsubstituted C6 to C60 aryl group; or a substituted or unsubstituted C2 to C60 heteroaryl group;
m1 is an integer of 0 to 4, and when m1 is 2 or greater, R21s are the same as or different from each other;
n1 is an integer of 0 to 3, and when n1 is 2 or greater, R22s are the same as or different from each other;
L21 to L23 are the same as or different from each other, and each independently a direct bond; a substituted or unsubstituted C6 to C60 arylene group; or a substituted or unsubstituted C2 to C60 heteroarylene group;
p is an integer of 0 to 5, and when p is 2 or greater, L21s are the same as or different from each other;
q is an integer of 0 to 5, and when q is 2 or greater, L22s are the same as or different from each other;
r is an integer of 0 to 5, and when r is 2 or greater, L23s are the same as or different from each other;
Ar21 to Ar23 are the same as or different from each other, and each independently a substituted or unsubstituted C6 to C60 aryl group; or a substituted or unsubstituted C2 to C60 heteroaryl group; and
s is 0 or 1.

16. The composition of claim 15, wherein the heterocyclic compound and the heterocyclic compound represented by Chemical Formula 2 have a weight ratio of 1:9 to 9:1.
